(19) United States
(12) Patent Application Publication
(10) Pub. No.: US 2026/0274833 A1
(43) Pub. Date: Sep. 17, 2026

(54) SULFAMIDE DERIVATIVES AS CYCLIN-DEPENDENT KINASE 2 INHIBITORS

(71) Applicant: NiKang Therapeutics, Inc., Wilmington, DE (US)

(72) Inventors: Yan LOU, Wilmington, DE (US); Zhiyong YU, Wilmington, DE (US)

(73) Assignee: NiKang Therapeutics, Inc., Wilmington, DE (US)

(21) Appl. No.: 18/871,829

(22) PCT Filed: Jun. 2, 2023
(86) PCT No.: PCT/US2023/067845

§ 371 Date: Dec. 5, 2024

Related U.S. Application Data
(60) Provisional application No. 63/366,033, filed on Jun. 8, 2022.

(51) Int. Cl. C07D 417/14; A61K 31/506; A61K 31/5377; A61K 31/5513; A61K 31/553; C07D 451/04; C07D 471/04; C07D 471/08; C07D 487/08; C07D 498/08; C07D 519/00

(52) U.S. Cl. CPC C07D 417/14; A61K 31/506; A61K 31/5377; A61K 31/5513; A61K 31/553; C07D 451/04; C07D 471/04; C07D 471/08; C07D 487/08; C07D 498/08; C07D 519/00

(57) ABSTRACT

The present disclosure provides certain sulfamide derivatives that are cyclin-dependent kinase 2 (CDK2) inhibitors of Formula (I) for the treatment of diseases treatable by inhibition of CDK2. Also provided are pharmaceutical compositions containing such compounds and processes for preparing such compounds.

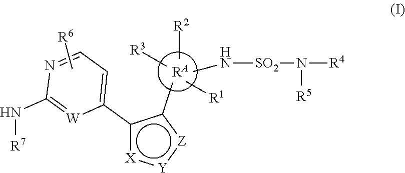

This application claims priority to U.S. Provisional Application No. 63/366,033, filed on Jun. 8, 2022, the contents of which are incorporated herein by reference in their entirety.

### FIELD OF THE DISCLOSURE

The present disclosure provides certain sulfamide derivatives that are cyclin-dependent kinase 2 (CDK2) inhibitors and are useful for the treatment of diseases mediated by the inhibition of CDK2. Also provided herein are pharmaceutical compositions containing such compounds and processes for preparing such compounds.

### BACKGROUND

Cyclin-dependent kinases (CDKs) are cellular kinases that are critical for orchestrating signaling events such as DNA replication and protein synthesis to ensure faithful eukaryotic cell division and proliferation. To achieve activation, the cyclin-dependent kinase catalytic units of CDK often require binding with regulatory subunits known as cyclins. In addition, the activity of CDK is also controlled by its phosphorylation status, as well as by binding of inhibitory proteins.

Of the CDKs identified thus far, at least CDK1/Cyclin B, CDK2/Cyclin E, CDK2/Cyclin A, CDK4/Cyclin D, and CDK6/Cyclin D complexes are known to be important regulators of cell cycle progression; while other CDKs are important in regulating gene transcription, DNA repair, differentiation, and apoptosis (see Morgan, D. O. Annu. Rev. Cell. Dev. Biol. (1997) 13:261-291).

Due to their key roles in regulating cell cycle and other essential cellular processes, increased activity or temporally abnormal activation of CDKs has been shown to result in the development of various types of cancer. CDK2/Cyclin E complex plays an important role in regulation of the G1/S transition, histone biosynthesis and centrosome duplication. Following the initial phosphorylation of retinoblastoma (Rb) by CDK 4/6/cyclin D, CDK2/Cyclin E further hyper-phosphorylates p-RB, releases G1 transcription factor, E2F, to transcribe genes required for S-phase entry. During S-phase, Cyclin E is degraded and CDK2 forms a complex with Cyclin A to promote phosphorylation of substrates that permit DNA replication and inactivation of E2F, for S-phase completion (see Asghar et al. Nat. Rev. Drug. Discov. (2015) 14: 130-146). In addition to cyclin bindings, the activity of CDK2 is also tightly regulated through its interaction with negative regulators, such as p21 and p27. In response to mitogenic stimulation, which signals optimal environment for cell cycle, p21 and p27 are phosphorylated and degraded, releasing the break on CDK2/Cyclin activation.

Cyclin E, the regulatory cyclin for CDK2, is frequently overexpressed in cancer, and its overexpression correlates with poor prognosis. For example, Cyclin E amplification or overexpression has been shown to associate with poor outcomes in breast cancer (see Keyomarsi et al., N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4/6 inhibitors in tamoxifen resistant and CCNE2 overexpressing cells. (see Caldon et al., Mol Cancer Ther. (2012) 11:1488-99; and Herrera-Abreu et al., Cancer Res. (2016) 76:2301-2313). Cyclin E amplification also reportedly contributes to trastuzumab resistance in HER2+ breast cancer. (see Scaltriti et al. Proc Natl Acad Sci. (2011) 108:3761-6). Cyclin E overexpression has also been reported to play a role in basal-like and triple negative breast cancer (TNBC), as well as inflammatory breast cancer (see Elsawaf Z. et al. Breast Care (2011) 6:273-278; and Alexander A. et al. Oncotarget (2017) 8:14897-14911.)

Amplification or overexpression of cyclin E1 (CCNE1) is also frequently found in ovarian, gastric, endometrial, uterus, bladder, esophagus, prostate, lung and other types of cancers (see Nakayama et al. Cancer (2010) 116:2621-34; Etemadmoghadam et al. Clin Cancer Res (2013) 19; 5960-71; Au-Yeung et al. Clin. Cancer Res. (2017) 23: 1862~1874; Ayhan et al. Modern Pathology (2017) 30:297-303; Ooi et al. Hum Pathol. (2017) 61:58-67; and Noske et al. Oncotarget (2017) 8:14794-14805) and often correlates with poor clinical outcomes.

The turnover of cyclin E1 is regulated by the SCF^{Fbxw7} ubiquitin E3 ligase component FBXW7 and the deubiquitinase USP28, which are frequently dysregulated in cancer. Loss-of-function mutations in FBXW7 or overexpression of USP28 lead to cyclin E overexpression and CDK2 activation (Welcker, M. & Clurman, B. E. 2008 Nat. Rev. Cancer 8, 83; Diefenbacher, M. E. et al. (2014) J. Clin. Invest. 124, 3407-3418). Alternatively, certain cancer cells express a hyperactive, truncated form of cyclin E (Caruso J A et al. Cancer Res. 2018 Oct. 1; 78(19):5481-5491). In addition, cyclin A amplification and overexpression have also been reported in various cancers such as hepatocellular carcinomas (Bayard, Q., et al. Nat Commun 9, 5235 (2018)), colorectal and breast cancers.

In contrast to the frequent upregulation of Cyclin E, the inhibitory regulators of CDK2, p21 and p27 are often abnormally downregulated in cancers. SKP2, a component of the SKP1-CUL1-F-box (SCF) complex, has also been implicated in tumorigenesis owing to its capacity to degrade p27 (Zhen Cai et al., (2020) Seminars in Cancer Biology 67 (2): 16-33). It is postulated that the loss or decrease of p21/p27 or overexpression of SKP2 lead to high and/or abnormal temporal activation of CDK2, thereby promoting oncogenic growth.

In addition, CDC25A and CDC25B, protein phosphatases responsible for the dephosphorylations that activate the CDK2, are overexpressed in various tumors. These various mechanisms of CDK2 activation have been validated using mouse cancer models.

The retinoblastoma (Rb) protein functions as transcription co-repressor and represents the key substrate of CDK4/6 and CDK2 complexes in driving cell division. Consistent with this canonical model, Rb-deficient tumors do not depend on CDK4/6 and are uniformly resistant to CDK4/6 inhibitors. However, an analysis on DepMAP (McFarland et al., 2018; Tsherniak et al., 2017), a database to assess the functional requirements of genes across 717 cancer cell lines using CRISPR technology, discovered that Rb-deficiency and high CDKN2A were associated with higher vulnerability to CDK2 or Cyclin E1 inhibition (Erik S. Knudsen et al. Cell Reports Mar. 1, 2022, 38:110448). It was proposed that CDK2/Cyclin E1 drives the phosphorylation of p130 to enable cell-cycle progression in this Rb-deficient CDK2/Cyclin E1-dependent setting.

Furthermore, CDK2/cyclin E phosphorylates oncogenic Myc to oppose ras-induced senescence, highlighting the importance of CDK2 in myc/ras-induced tumorigenesis (Per Hydbring, PNAS Jan. 5, 2010, 107 (1) 58-63; Campaner, S., Doni, M., Hydbring, P. et al. Nat Cell Biol 12, 54-59 (2010)). Inactivation of CDK2 has been shown to be synthetically lethal to myc over-expressing cancer cells (Jan J. Molenaar, PNAS Aug. 4, 2009 106 (31) 12968-12973; Sara Bolin et al., Oncogene 37:2850-2862 (2018)).

Centrosomal protein CP110 plays an important role in centrosome duplication/separation and requires CDK2 phosphorylation to induce centrosome clustering (reviewed in M Kawakami et al., (2018) Mol Cancer Ther 17(4):724-731). Aneuploidy cancer cells are genetically unstable and often have supernumerary centrosomes. If centrosome clustering is blocked, aneuploid cells with supernumerary centrosomes undergo multipolar division, leading to apoptosis of daughter cells, a process called anaphase catastrophe. In addition, CP110 is downregulated in KRAS-mutant lung cancer, enhancing sensitivity of this cancer to CDK2 inhibitors. In models of KRAS-mutant lung cancer, CDK2 inhibition resulted in anaphase catastrophe and apoptosis and reduced growth of lung cancer xenografts. Because aneuploid cells with supernumerary centrosomes occur in many cancers, there might be potential to extend CDK2 inhibitors to other settings beyond KRAS-mutant lung cancer.

CDK2 was also shown to play a role in blocking myeloid differentiation in AML (Meidan Ying et al. Blood 2018 Jun. 14; 131(24):2698-2711). Inhibiting CDK2 effectively induced granulocytic differentiation in AML cell lines and arrested tumor growth in AML mice models. Synergetic effect was demonstrated for combining CDK2 inhibition and all-trans-retinoic acid (ATRA) in AML both in vitro and in vivo (Xuejing Shao et al., Pharmacol Res, 2020 151: 104545).

Pharmacologic inhibition or genetic deletion of CDK2 has also been shown to preserve hearing function in animal models treated with cisplatin or noise (see Teitz T et al. J Exp Med. 2018 Apr. 2; 215(4):1187-1203). Therefore, in addition to anti-tumor therapies, CDK2 inhibition can also be used as a promising preventive treatment for noise-, cisplatin-, or antibiotic-induced or age-related hearing loss, for which no Food and Drug Administration approved drugs are currently available.

Given the role of CDK2 in human malignancy, there is a need for CDK2 inhibitors for the treatment of cancers and related diseases. The present disclosure fulfills this and related needs.

### SUMMARY

In a first aspect, provided is a compound of Formula (I):

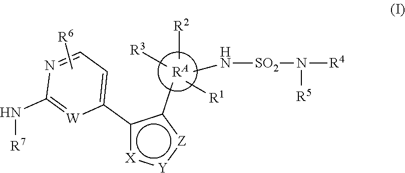

wherein:
W is N or CH (or C when attached to R⁶);
X and Z are independently CR⁸, NR^{8A}, N, O, or S; and Y is CR⁹, NR¹⁰, or N; provided that both of X and Z are not simultaneously O or S; and at least one of X, Y, and Z is other than carbon;
R⁸ is hydrogen, alkyl, halo, hydroxyalkyl, or cyano;
R^{8A} is hydrogen or alkyl;
R⁹ and R¹⁰ are independently hydrogen, alkyl, haloalkyl, hydroxyalkyl, alkoxyalkyl, cyanoalkyl, alkylsulfonyl, alkylsulfonylalkyl, amino, alkylamino, dialkylamino, substituted amino, aminoalkyl, cycloalkyl, bicyclic cycloalkyl, bridged cycloalkyl, spiro cycloalkyl, heterocyclyl, bicyclic heterocyclyl, bridged heterocyclyl, spiro heterocyclyl, aryl, aralkyl, heteroaryl, or heteroaralkyl, wherein:
   (A) cycloalkyl, bicyclic cycloalkyl, bridged cycloalkyl, and spiro cycloalkyl of R⁹ and R¹⁰ are substituted with one or two R^{a} independently selected from hydrogen, deuterium, alkyl, deuteroalkyl, cycloalkyl, halo, haloalkyl, hydroxy, hydroxyalkyl, cyano, amino, alkylamino, and dialkylamino;
   (B) heterocyclyl, bicyclic heterocyclyl, bridged heterocyclyl, and spiro heterocyclyl of R⁹ and R¹⁰ are substituted with R^{b}, R^{c}, and R^{d} independently selected from hydrogen, alkyl, deuteroalkyl, cycloalkyl, bridged cycloalkyl, spiro cycloalkyl (wherein cycloalkyl, bridged cycloalkyl, and spiro cycloalkyl are substituted with one or two substituents independently selected from hydrogen, alkyl, halo, hydroxy, and cyano), alkoxy, halo, haloalkyl, haloalkoxy, alkoxycarbonyl, hydroxy, cyano, amino, alkylamino, dialkylamino, aryl, aralkyl, heterocyclyl, bridged heterocyclyl, spiro heterocyclyl, heteroaryl, or heteroaralkyl (wherein aryl, by itself or as part of aralkyl, heteroaryl, by itself or as part of heteroaralkyl, heterocyclyl, bridged heterocyclyl, and spiro heterocyclyl are substituted with R^{e}, R^{f}, and R^{g} independently selected from hydrogen, alkyl, cycloalkyl, halo, haloalkyl, alkoxy, haloalkoxy, hydroxy, cyano, amino, alkylamino, and dialkylamino); and
   (C) aryl, by itself or as part of aralkyl, and heteroaryl, by itself or as part of heteroaralkyl, of R⁹ and R¹⁰ are substituted with R^{h}, R^{j}, and R^{k} independently selected from hydrogen, alkyl, deuteroalkyl, cycloalkyl (wherein cycloalkyl is optionally substituted with one or two substituents independently selected from alkyl, halo, hydroxy, and cyano), alkoxy, halo, haloalkyl, haloalkoxy, alkoxycarbonyl, hydroxy, cyano, amino, alkylamino, dialkylamino, aryl, aralkyl, heterocyclyl, bridged heterocyclyl, spiro heterocyclyl, heteroaryl, or heteroaralkyl (wherein aryl, by itself or as part of aralkyl, heteroaryl, by itself or as part of heteroaralkyl, heterocyclyl, bridged heterocyclyl, and spiro heterocyclyl are substituted with R^{m}, Rⁿ, and R^{o} independently selected from hydrogen, alkyl, cycloalkyl, halo, haloalkyl, alkoxy, haloalkoxy, hydroxy, cyano, amino, alkylamino, and dialkylamino);
ring R^{A} is phenyl or heteroaryl;
R¹, R², and R³ are independently hydrogen, deuterium, alkyl, halo, haloalkyl, haloalkoxy, alkoxy, hydroxy, hydroxyalkyl, alkoxyalkyl, aminoalkyl, or cyano;
R⁴ and R⁵ are independently hydrogen, alkyl, haloalkyl, alkoxyalkyl, hydroxalkyl, aminoalkyl, cycloalkyl, cycloalkylalkyl, fused cycloalkyl, aryl, aralkyl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, and spiro heterocyclylalkyl (wherein cycloalkyl, by itself or as part of cycloalkylalkyl, fused cycloalkyl, aryl, by itself or as part of aralkyl, heteroaryl, by itself or as part of heteroaralkyl, heterocyclyl, by itself or as part of heterocyclylalkyl, bicyclic heterocyclyl, by itself or as part of bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{p}, R^{q}, and R^{r} independently from hydrogen, alkyl, alkoxy, alkylsulfonyl, alkyloxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, aminoalkylamino, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, and heterocyclylalkyloxy (wherein aryl, by itself or as part of aralkyl and aryloxy, heteroaryl, by itself or as part of heteroaryloxy and heteroaralkyl, and heterocyclyl, by itself or as part of heterocyclylalkyl, heterocyclyloxy, and heterocyclylalkyloxy, are substituted with one, two, or three substituents independently selected from hydrogen, alkyl, alkoxy, hydroxy, halo, haloalkyl, haloalkoxy, and cyano)); or
R⁴ and R⁵ together with the nitrogen atom to which they are attached form a ring selected from cyclylaminyl, bicyclylaminyl, fused cyclylaminyl, bridged cyclylaminyl, fused bridged cyclylaminyl, spiroaminyl, and fused spiroaminyl, wherein each of the aforementioned rings is substituted with R^{s}, R^{t}, and R^{u} independently selected from hydrogen, deuterium, alkyl, alkoxy, alkylsulfonyl, alkoxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, aminoalkylamino, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, heterocyclylalkyloxy, and unsaturated heterocyclyl (wherein aryl, by itself or as part of aryloxy and aralkyl, heteroaryl, by itself or as part of heteroaryloxy and heteroaralkyl, heterocyclyl, by itself or as part of heterocyclylalkyl, heterocyclyloxy, and heterocyclylalkyloxy, and unsaturated heterocyclyl are substituted with one to three substituents independently selected from hydrogen, alkyl, alkoxy, hydroxy, halo, haloalkyl, haloalkoxy, and cyano);
R⁶ is hydrogen, alkyl, cycloalkyl, cyano, halo, or haloalkyl;
R⁷ is -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, -Q⁶-(alk⁷)ₙ₇-NR²¹SO₂R²², heterocyclyl, bicyclic heterocyclyl, fused heterocyclyl, spiro heterocyclyl, bridged heterocyclyl, cyclylsulfoximinyl, bicyclylsulfoximinyl, spiro sulfoximinyl, aryl, heteroaryl, or fused heteroaryl wherein:
each of Q, Q¹, Q², Q³, Q⁴, Q⁵, and Q⁶ is a bond, cycloalkyl, bridged cycloalkyl, spiro cycloalkyl, heterocyclyl^{A}, bicyclic heterocyclyl^{A}, bridged heterocyclyl^{A}, fused heterocyclyl^{A}, spiro heterocyclyl^{A}, aryl, or heteroaryl, wherein each of the aforementioned rings is substituted with R^{v} and R^{w} independently selected from hydrogen, deuterium, alkyl, halo, haloalkyl, haloalkoxy, alkoxy, and cyano;
each of n¹, n², n³, n⁴, n⁵, n⁶, and n⁷ is 0 or 1, provided that, when Q is a bond, then each of n¹, n², n³, n⁴, n⁵, n⁶, and n⁷ is 1;
each alk¹, alk², alk³, alk⁴, alk⁵, alk⁶, and alk⁷ is alkylene;
R¹², R¹⁹, and R²¹ are hydrogen or alkyl;
R¹¹, R¹³, R¹⁶, R²⁰, and R²¹ are independently selected from hydrogen, alkyl, fluoro, chloro, bromo, haloalkyl, hydroxyalkyl, alkoxyalkyl, aminoalkyl, cycloalkyl, cycloalkylalkyl, aryl, aralkyl, heteroaryl, heteroaralkyl, heterocyclyl, bicyclic heterocyclyl, bridged heterocyclyl, fused heterocyclyl, spiro heterocyclyl, heterocyclylalkyl, or —CR²³=CR²⁴R²⁵ [where R²³ is hydrogen, alkyl, or cyano, R²⁴ is hydrogen or alkyl, and R²⁵ is hydrogen, alkyl, halo, haloalkyl, alkoxyalkyl, hydroxyalkyl, amino, alkylamino, dialkylamino, or -(alkylene)-NR²⁶R²⁷ (where R²⁶ and R²⁷ are independently hydrogen, or alkyl), or heterocyclylalkyl], wherein cycloalkyl, by itself or as part of cycloalkylalkyl, aryl, by itself or as part of aralkyl, heteroaryl, by itself or as part of heteroaralkyl, heterocyclyl, by itself or as part of heterocyclylalkyl, bicyclic heterocyclyl, bridged heterocyclyl, fused heterocyclyl, and spiro heterocyclyl of R¹¹, R¹³, R¹⁶, R²⁰ and R²² and heterocyclyl of heterocyclylalkyl of R²⁵ are substituted with one to three substituents independently selected from hydrogen, alkyl, alkoxy, hydroxy, halo, haloalkyl, haloalkoxy, hydroxyalkyl, alkoxyalkyl, aminoalkyl, cyano, and heterocyclyl; provided R¹¹, R¹⁶, R²⁰, and R²² are not hydrogen;
R¹⁴, R¹⁵, R¹⁷, and R¹⁸ are independently selected from hydrogen, alkyl, haloalkyl, hydroxyalkyl, alkoxyalkyl, aminoalkyl, cycloalkyl, cycloalkylalkyl, aryl, aralkyl, heteroaryl, heteroaralkyl, heterocyclyl, or heterocyclylalkyl, wherein cycloalkyl, by itself or as part of cycloalkylalkyl, aryl, by itself or as part of aralkyl, heteroaryl, by itself or as part of heteroaralkyl, and heterocyclyl, by itself or as part heterocyclylalkyl, are substituted with one to three substituents independently selected from hydrogen, alkyl, alkoxy, hydroxy, halo, haloalkyl, haloalkoxy, and cyano; and
the heterocyclyl, bicyclic heterocyclyl, fused heterocyclyl, spiro heterocyclyl, bridged heterocyclyl, cyclylsulfoximinyl, spiro cyclylsulfoximinyl, aryl, heteroaryl, and fused heteroaryl of R⁷ are substituted with R^{x}, R^{y}, and R^{y1} independently selected from hydrogen, deuterium, alkyl, alkoxy, alkoxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, or heterocyclylalkyloxy (wherein aryl, by itself or as part of aralkyl and aryloxy, heteroaryl, by itself or as part, of heteroaryloxy and heteroaralkyl, and heterocyclyl, by itself or as part of, heterocyclylalkyl, heterocyclyloxy, and heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl);
   or a pharmaceutically acceptable salt thereof;
provided that the compound of Formula (I) is not:
N-(3-(2-isopropyl-5-(2-((2-(methylsulfonyl)ethyl)amino)pyrimidin-4-yl)thiazol-4-yl)phenyl)-morpholine-4-sulfonamide; or pharmaceutically acceptable salts thereof.

In a second aspect, provided is a pharmaceutical composition comprising a compound of Formula (I) (or any of the embodiments thereof described herein), or a pharmaceutically acceptable salt thereof, and a pharmaceutically acceptable excipient.

In general, compounds of Formula (I) selectively inhibit CDK2 over CDK1 and can also be selective over CDK4 and/or CDK6. As such, in a third aspect, provided is a method of treating a disease treatable by inhibition of CDK2 in a patient, preferably the patient is in need of such treatment, which method comprises administering to the patient, preferably a patient in need of such treatment, a therapeutically effective amount of a compound of Formula (I) (or any of the embodiments thereof described herein), or a pharmaceutically acceptable salt thereof, or a pharmaceutical composition thereof as disclosed herein.

In a first embodiment of the third aspect, the disease is cancer. In a second embodiment of the third aspect, the disease is cancer selected from ovarian cancer, endometrial cancer, breast cancer (e.g., hormone receptor-positive breast cancer and triple-negative breast cancer), lung cancer (e.g., adenocarcinoma, small cell lung cancer, non-small cell lung carcinomas, parvicellular and non-parvicellular carcinoma, bronchial carcinoma, bronchial adenoma, and/or pleuropulmonary blastoma), skin cancer (e.g. melanoma, squamous cell carcinoma, Kaposi sarcoma, and/or Merkel cell skin cancer), bladder cancer, cervical cancer, colorectal cancer, cancer of the small intestine, colon cancer, rectal cancer, cancer of the anus, gastric cancer, head and neck cancer (e.g., cancers of the larynx, hypopharynx, nasopharynx, oropharynx, lips, and/or mouth), liver cancer (e.g., hepatocellular carcinoma, cholangiocellular carcinoma), prostate cancer, testicular cancer, uterine cancer, esophageal cancer, gall bladder cancer, pancreatic cancer (e.g. exocrine pancreatic carcinoma), stomach cancer, thyroid cancer, and parathyroid cancer. In a third embodiment of the third aspect, the disease is cancer that are resistant to CDK4/6 inhibitors through CDK2-mediated mechanisms.

In a fourth aspect, provided is a method of treating noise-, cisplatin- or antibiotic-induced- or age-related hearing loss, which method comprises administering to the patient, preferably a patient in need of such treatment, a therapeutically effective amount of a compound of Formula (I) (or any of the embodiments thereof described herein), or a pharmaceutically acceptable salt thereof, or a pharmaceutical composition thereof as disclosed herein. In some embodiments, the amount of hearing loss is reduced when compared to an age-matched control. In some embodiments, the hearing loss is prevented when compared to an age-matched control.

In a fifth aspect, provided is a compound of Formula (I) (or any of the embodiments thereof described herein), or a pharmaceutically acceptable salt thereof for use as a medicament. In one embodiment of the fourth aspect, the compound Formula (I) (and any of the embodiments thereof described herein), or a pharmaceutically acceptable salt thereof is useful for the treatment of one or more of diseases disclosed in the third and/or fourth aspects above, including embodiments therein.

In a sixth aspect, provided is the use of a compound of Formula (I) (and any of the embodiments thereof disclosed herein)), or a pharmaceutically acceptable salt thereof in the manufacture of a medicament for treating a disease in a patient treatable by inhibition of CDK2. In an embodiment of the sixth aspect, the disease is one or more of diseases disclosed in the third and/or fourth aspects above (including embodiments therein).

In a seventh aspect, provided is a method of inhibiting CDK2 which method comprises contacting CDK2 with a compound of Formula (I) (and any of the embodiments thereof described herein), or a pharmaceutically acceptable salt thereof; or contacting CDK2 with a pharmaceutical composition comprising a compound of Formula (I) (and any of the embodiments thereof described herein), or a pharmaceutically acceptable salt thereof, and a pharmaceutically acceptable excipient. In one embodiment, the CDK2 is contacted with a compound of Formula (I) (or any of the embodiments thereof described herein), or a pharmaceutically acceptable salt thereof, in vitro. In another embodiment, the CDK2 is contacted with a compound of Formula (I) (or any of the embodiments thereof described herein), or a pharmaceutically acceptable salt thereof, in vivo.

In any of the aforementioned aspects involving the treatment of cancer, are further embodiments comprising administering the compound of Formula (I) (or any of the embodiments thereof disclosed herein), or a pharmaceutically acceptable salt thereof in combination with at least one additional anticancer agent. When combination therapy is used, the agents can be administered simultaneously or sequentially.

### DETAILED DESCRIPTION

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated; e.g., methylene, ethylene, propylene, I-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or feri-butoxy, and the like.

“Alkoxyalkyl” means a linear monovalent hydrocarbon radical of one to six carbon atoms or a branched monovalent hydrocarbon radical of three to six carbons substituted with one alkoxy group as defined above, e.g., 2-methoxyethyl, 1-, 2-, or 3-methoxypropyl, 2-ethoxyethyl, and the like.

“Alkoxyalkyloxy” means a —OR^{z} where R^{z} is alkoxyalkyl as defined above.

Representative examples include, but are not limited to, 2-methoxyethyloxy, 1-, 2-, or 3-methoxypropyloxy, 2-ethoxyethyloxy, and the like.

“Alkoxyalkyloxyalkyl” means a -(alkylene)-R^{z} where R^{z} is alkoxyalkyloxy as defined above. Representative examples include, but are not limited to, 2-methoxyethyloxymethyl, 1-, 2-, or 3-methoxypropyloxymethyl, 2-ethoxyethyloxyethyl, and the like.

“Alkoxycarbonyl” or “alkyloxycarbonyl” means a —C(O)OR^{z} radical where R^{z} is alkyl as defined above, e.g., ethoxycarbonyl, ethoxycarbonyl, and the like.

“Alkoxycarbonylamino” means a —NHC(O)OR^{z} radical where R^{z} is alkyl as defined above, e.g., ethoxycarbonylamino, ethoxycarbonylamino, and the like.

“Alkoxycarbonylaminoalkyl” means a -(alkylene)-R^{z} radical where R^{z} is alkoxycarbonylamino as defined above, e.g., ethoxycarbonylaminomethyl, ethoxycarbonylaminoethyl, and the like.

“Alkylsulfonyl” means a —SO₂R^{z} radical where R^{z} is alkyl as defined above, e.g., methylsulfonyl, ethylsulfonyl, and the like.

“Alkylsulfonylalkyl” means a linear monovalent hydrocarbon radical of one to six carbon atoms or a branched monovalent hydrocarbon radical of three to six carbons substituted with one alkylsulfonyl group as defined above, e.g., 2-methylsulfonylmethyl, methylsulfonylethyl, 2-ethylsulfonylethyl, and the like.

“Amino” means a —NH₂.

“Alkylamino” means a —NHR where R is alkyl as defined above e.g., methylamino, ethylamino, propylamino, and the like.

“Aminoalkyl” means a linear monovalent hydrocarbon radical of one to six carbon atoms or a branched monovalent hydrocarbon radical of three to six carbons substituted with —NR^{z}′R^{z}″ where R^{z}′ and R^{z}″ are independently hydrogen, alkyl, deuteroalkyl, cycloalkyl, cycloalkylalkyl (wherein cycloalkyl and cycloalkyl ring in cycloalkylalkyl are optionally substituted with one, two, or three substituents independently selected from alkyl, haloalkyl, halo, hydroxy, alkoxy, and cyano), haloalkyl, hydroxyalkyl, alkoxyalkyl, aminocarbonylalkyl, alkylsulfonylalkyl, alkylcarbonyl, alkoxycarbonyl, optionally substituted aryl, optionally substituted heteroaryl, optionally substituted heteroaralkyl, optionally substituted heterocyclyl, or optionally substituted heterocyclylalkyl, each as defined herein. Representative examples include, but are not limited to, aminomethyl, aminoethyl, methylaminomethyl, and the like.

“Aminoalkyloxy” or “aminoalkoxy” means a —OR^{z} where R^{z} is aminoalkyl as defined above. Representative examples include, but are not limited to, aminomethyloxy, aminoethyloxy, methylaminomethyloxy, dimethylaminoethyloxy, and the like.

“Aminoalkylamino” means a —NR-(alkylene)-NR^{z}′R^{z}″ where R^{z}′ and R^{z}″ are independently hydrogen, alkyl, deuteroalkyl, cycloalkyl, cycloalkylalkyl (wherein cycloalkyl and cycloalkyl ring in cycloalkylalkyl is optionally substituted with one, two, or three substituents independently selected from alkyl, haloalkyl, halo, hydroxy, alkoxy, and cyano), haloalkyl, hydroxyalkyl, alkoxyalkyl, aminocarbonylalkyl, alkylsulfonylalkyl, alkylcarbonyl, optionally substituted aryl, optionally substituted heteroaryl, optionally substituted heteroaralkyl, optionally substituted heterocyclyl, or optionally substituted heterocyclylalkyl, each as defined herein. Representative examples include, but are not limited to, aminoethylamino, methylaminoethylamino, diethylaminoethylamino, and the like.

“Aminocarbonyl” means a —CONR^{z}′R^{z}″ radical where R^{z}′ and R^{z}″ are independently hydrogen, alkyl, cycloalkyl (optionally substituted with one, two, or three substituents independently selected from alkyl, halo, hydroxy, alkoxy, or cyano), haloalkyl, hydroxyalkyl, alkoxyalkyl, and alkylcarbonyl, each as defined herein, e.g., aminocarbonyl, methylaminocarbonyl, dimethylaminocarbonyl, and the like.

“Aminocarbonylalkyl” means a -(alkylene)-CONR^{z}′R^{z}″ radical where R^{z}′ and R^{z}″ are independently hydrogen, alkyl, cycloalkyl (optionally substituted with one, two, or three substituents independently selected from alkyl, halo, hydroxy, alkoxy, or cyano), haloalkyl, hydroxyalkyl, alkoxyalkyl, and alkylcarbonyl, each as defined herein.

“Acyl” means a —C(O)R^{z} radical where R^{z} is alkyl, haloalkyl, cycloalkyl, optionally substituted phenyl, optionally substituted heteroaryl, or optionally substituted heterocyclyl, as defined herein. Representative examples include, but are not limited to, methylcarbonyl, ethylcarbonyl, benzoyl, trifluoromethylcarbonyl, cyclopropylcarbonyl, and the like. When R^{z} is alkyl, acyl is also referred to herein as alkylcarbonyl.

“Aryl” means a monovalent monocyclic or bicyclic aromatic hydrocarbon radical of 6 to 10 ring atoms e.g., phenyl or naphthyl.

“Aryloxy” means a —OR^{z} radical where R^{z} is aryl as defined above. Representative examples include, phenoxy, naphthyloxy.

“Aralkyl” means a -(alkylene)-R^{z} radical where R^{z} is aryl as defined above. Examples include, but are not limited to, benzyl, phenethyl, and the like.

“Bicyclic cycloalkyl” means a saturated monovalent bicyclic hydrocarbon radical of 5 to 10 carbon atoms where the two rings are fused at two adjacent carbon ring atoms. Unless otherwise stated, bicyclic cycloalkyl is optionally substituted with one or two substituents independently selected from deuterium, alkyl, halo, haloalkyl, alkoxy, hydroxy, and cyano, Examples include, but are not limited to, bicyclo[3.1.0]hexan-6-yl, and the like.

“Bicyclylaminyl” means a saturated monovalent bicyclic ring of 5 to 10 ring atoms where the two rings are fused at two adjacent ring atoms and in which one ring atom is nitrogen and an additional ring atom can be a heteroatom independently selected from N, O, and S(O), where n is an integer from 0 to 2, the remaining ring atoms being C. Additionally, one or two ring carbon atoms of the bicycloaminyl group can optionally be replaced by a —CO— group. Representative examples include, but is not limited to, 3-azabicyclo[3.1.0]hexan-3-yl, 3-azabicyclo[4.1.0]heptan-3-yl, and the like.

“Bicyclic heterocyclyl” means a saturated monovalent bicyclic group of 5 to 10 ring atoms where the two rings are fused at two adjacent ring atoms and in which one or two ring atoms are heteroatom independently selected from N, O, and S(O)ₙ, where n is an integer selected from 0 to 2, the remaining ring atoms being C, unless stated otherwise. Additionally, one or two ring carbon atoms of the bicyclic heterocyclyl can optionally be replaced by a —CO— group. When the bicyclic heterocyclyl contains only S(O)₂, it may be referred to herein as bicyclylsulfonyl. More specifically the term bicyclic heterocyclyl includes, but is not limited to, hexahydrofuro[3,2-b]furanyl, hexahydro-1H-pyrrolizinyl, 3,3-dioxido-3-thiabicyclo[3.1.0]hexan-6-yl, and the like. When bicyclic heterocyclyl ring as defined above, contains only —SO₂— in the ring, it is also referred to herein “bicyclylsulfonyl” and is a subset of the bicyclic heterocyclyl ring.

“Bicyclic heterocyclylalkyl” means -(alkylene)-R radical where R is bicyclyl heterocyclyl as defined above.

“Bicyclic heterocyclyl^{A}” means a saturated monovalent bicyclic group of 5 to 10 ring atoms where the two rings are fused at two adjacent ring atoms and in which one or two ring atoms are heteroatom independently selected from N and O, the remaining ring atoms being C, unless stated otherwise. Additionally, one or two ring carbon atoms of the bicyclic heterocyclyl can optionally be replaced by a —CO— group. The term bicyclic heterocyclyl^{A} includes, but is not limited to, 3-azabicyclo[3.1.0]hexan-6-yl, and the like.

“Bicyclylsulfoximinyl” means a saturated monovalent bicyclic ring of 4 to 8 ring atoms where the two rings are fused at two adjacent ring atoms and in which one ring atom is >S(═O))(NR^{z}) where R^{z} is hydrogen or alkyl, as defined herein, the remaining ring atoms being C. Representative examples of bicyclylsulfoximinyl include, but is not limited to, 1-imino-3-oxohexahydro-3λ⁶-thiobicyclo[3.1.0]hexan-6-yl, octahydrocyclopenta[c]pyrrol-5-yl, and the like.

“Bridged cycloalkyl” means a saturated monovalent bicyclic or tricyclic hydrocarbon radical having 5 to 10 ring carbon ring atoms in which one or two ring atoms are linked by a (CR^{z}R^{z1})n group (where n is an integer selected from 1 to 3 and R^{z} and R^{z1} are independently H of methyl) to a non-adjacent ring atom(s) (also may be referred to herein as “bridging” group). For clarity, when the bridged cycloalkyl is a bicylic ring, it has one bridging group and when the bridged cycloalkyl is a tricylic ring, it has two bridging groups). Examples include, but are not limited to, bicyclo[1.1.1]pentyl, bicyclo[2.1.1]hexyl, bicyclo[2.2.2]-octyl, adamantyl, and the like.

“Bridged cyclylaminyl” means a saturated monovalent bicyclic ring of 5 to 10 ring atoms in which one ring atom is nitrogen and two non-adjacent ring atoms are linked by a (CRR′)m1 group where m1 is an integer from 1 to 3 and R and R′ are independently H or methyl (also may be referred to herein as “bridging” group) and further wherein an additional ring atom, including an atom in the bridging group, can be a heteroatom independently selected from N, O, and S(O)ₙ, where n is an integer from 0 to 2, the remaining ring atoms being C. Representative examples of bridged cyclylaminyl includes, but is not limited to, 2-azabicyclo[2.2.1]heptan-2-yl, 3-azabicyclo[3.1.1]heptan-3-yl, 3-azabicyclo[3.2.1]octan-3-yl, and the like.

“Bridged heterocyclyl” means a saturated monovalent bicyclic radical having 5 to 9 ring carbon ring atoms in which two non-adjacent ring atoms are linked by a (CR^{z}R^{z1})n group where n is 1 to 3 and R^{z} and R^{z1} are independently H or methyl (also may be referred to herein as “bridging” group) and further wherein one or two ring carbon atoms, including an atom in the bridging group, are replaced by a heteroatom selected from N, O, and S(O)ₙ, where n is an integer selected from 0 to 2. Examples include, but are not limited to, 2-azabicyclo[2.2.2]octyl, quinuclidinyl, 7-oxabicyclo[2.2.1]heptyl, and the like. When bridged heterocyclyl ring as defined above, contains only —SO₂— in the ring, it is also referred to herein “bridged cyclylsulfonyl” and is a subset of the bridged heterocyclyl ring.

“Bridged heterocyclylalkyl” means -(alkylene)-R radical where R is bridged heterocyclyl as defined above.

“Bridged heterocyclyl^{A}” means a saturated monovalent bicyclic radical having 5 to 9 ring carbon ring atoms in which two non-adjacent ring atoms are linked by a (CR^{z}R^{z″})n group where n is 1 to 3 and R^{z} and R^{z1} are independently H or methyl (also may be referred to herein as “bridging” group) and further wherein one or two ring carbon atoms, including an atom in the bridging group, are replaced by a heteroatom selected from N and O. Examples include, but are not limited to, 2-azabicyclo[2.2.2]octyl, quinuclidinyl, 7-oxabicyclo[2.2.1]heptyl, 6-azabicyclo[3.1.1]heptan-3-yl, 8-azabicyclo[3.2.1]octan-3-yl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkoxy” means a —OR radical where R is cycloalkyl as defined above. Representative examples include, but are not limited to, cyclopropyloxy, cyclopentyloxy, cyclohexyloxy, and the like.

“Cycloalkylalkyl” means an -(alkylene)-R^{z} radical where R^{z} is cycloalkyl as defined above. Examples include, but are not limited to, cyclopropylmethyl cyclobutylethyl, cyclopentylmethyl, cyclohexylmethyl, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Cyanoalkoxy” means a —OR radical where R is cyanoalkyl as defined above. Representative examples include, but are not limited to, cyanomethoxy, cyanoethoxy, and the like.

“Cyclylaminyl” means a saturated monovalent monocyclic radical of 4 to 8 ring atoms in which one ring atom is nitrogen and an additional ring atom can be a heteroatom selected from N, O, and S(O)ₙ, where n is an integer from 0 to 2, the remaining ring atoms being C, unless stated otherwise. Additionally, one or two ring carbon atoms in cyclylaminyl can optionally be replaced by a —C(═O)— group. Representative examples include, but is not limited to, pyrrolidinyl, piperidinyl, homopiperidinyl, 2-oxopyrrolidinyl, 2-oxopiperidinyl, morpholinyl, piperazinyl, thiomorpholinyl, and the like.

“Cyclylsulfoximinyl” means a saturated monovalent monocyclic ring of 4 to 8 ring atoms in which one ring atom is >S(═O)(NR^{z}) where R^{z} is hydrogen or alkyl, as defined herein, the remaining ring atoms being C. Representative examples of cyclylsulfoximinyl include, but is not limited to, 1-imino-1-oxohexahydro-126-thiopyran-4-yl, and the like.

“Deuterium” means 2H or D.

“Dialkylamino” means a —NRR′ where R and R′ are independently alkyl as defined above e.g., dimethylamino, diethylamino, methylpropylamino, and the like.

“Deuteroalkyl” means alkyl as defined above, which is substituted with one, two, or three deuterium e.g., trideuteromethyl.

“Fused bridged cyclylaminyl” as used herein, means bridged cycylaminyl as defined herein above where two adjacent ring atoms of the bridged cyclylaminyl are fused to two adjacent ring atoms of phenyl or five or six membered heteroaryl, each as defined herein, unless stated otherwise. The fused bridged cyclylaminyl can be attached at any atom of the ring valency permitting. Non limiting examples of the fused bridged cyclylaminyl include, 1,2,3,4-tetrahydro-1,3-epiminonaphth-9-yl, and the like.

“Fused cycloalkyl” means a three to six membered cycloalkyl, as defined above, where two adjacent carbon atoms of the cycloalkyl are fused to two adjacent ring atoms of phenyl or a five or six membered heteroaryl, as defined herein, unless stated otherwise. The fused cycloalkyl can be attached at any atom of the ring valency permitting. Non limiting examples of the fused cycloalkyl include 4,5,6,7-tetrahydroindazol-5-yl, and the like.

“Fused cyclylaminyl” as used herein, means cycylaminyl as defined herein above where two adjacent ring atoms of cyclylaminyl are fused to two adjacent ring atoms of phenyl or a five or six membered heteroaryl, each as defined herein, unless stated otherwise. The fused cyclylaminyl can be attached at any atom of the ring. Non limiting examples of the fused cyclylaminyl include 2,3-dihydrobenzo[b][1,4]-dioxinyl, 2-oxabicyclo[3.1.0]hexanyl, indolin-2-one-1-yl, indolinyl, isoindolyl, and the like.

“Fused heteroaryl” as used herein, means 5 or 6 membered heteroaryl as defined herein below where two adjacent ring atoms of the 5 or 6 membered heteroaryl are fused to two adjacent ring atoms of 5 to 7 membered cycloalkyl, as defined herein, unless stated otherwise. The fused heteroaryl can be attached at any atom of the ring. Non limiting examples of the fused heterocycloalkyl include 4,5,6,7-tetrahydro-1H-indole, 4,5,6,7-tetrahydro-1H-pyrazole, and the like.

“Fused heterocyclyl” as used herein, means heterocyclyl as defined herein below where two adjacent ring atoms of heterocyclyl are fused to two adjacent ring atoms of phenyl or five or six membered heteroaryl, each as defined herein, unless stated otherwise. The fused heterocyclyl can be attached at any atom of the ring. Non limiting examples of the fused heterocycloalkyl include 2,3-dihydrobenzo[b][1,4]-dioxinyl, 2-oxabicyclo[3.1.0]hexanyl, indolin-2-one-1-yl, indolinyl, isoindolyl, and the like.

“Fused heterocyclylalkyl” means -(alkylene)-R radical where R is fused heterocyclyl as defined above.

“Fused heterocyclyl^{A}” as used herein, means heterocyclyl^{A} as defined herein below where two adjacent ring atoms of heterocyclyl^{A} are fused to two adjacent ring atoms of phenyl or five or six membered heteroaryl, each as defined herein, unless stated otherwise. The fused heterocycly^{A} can be attached at any atom of the ring. Non limiting examples of the fused heterocyclyl^{A} include 2,3-dihydrobenzo[b][1,4]-dioxinyl, 2-oxabicyclo[3.1.0]hexanyl, indolin-2-one-1-yl, indolinyl, isoindolyl, and the like.

“Fused spiroaminyl” as used herein, means spiroaminyl as defined herein above where two adjacent ring atoms of spiroaminyl are fused to two adjacent ring atoms of phenyl or five or six membered heteroaryl, each as defined herein, unless stated otherwise. Non limiting examples of the fused spiroaminyl include

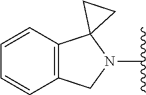 [SMILES: *N1Cc2ccccc2C12CC2]

and the like.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present, they are both not on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkyloxy” means a —OR^{z} where R^{z} is hydroxyalkyl as defined above. Representative example include, but are not limited to, 2-hydoxyethyloxy, 1-, 2-, or 3-hydroxypropyloxy, 2-hydroxyethyloxy, and the like.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N, O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms and is a monocyclic ring, it is also referred to herein as 5- or 6-membered monocyclic heteroaryl. When the heteroaryl ring contains 9 or 10 ring atoms and is a bicyclic ring, it is also referred to herein as 9- or 10-membered bicyclic heteroaryl.

“Heteroaryloxy” means a —OR^{z} radical where R^{z} is heteroaryl as defined above. Representative examples include, but are not limited to, pyridinyloxy, imidazolyloxy, pyrimidinyloxy, furanyloxy, benzimidazolyloxy, and the like.

“Heteroaralkyl” means a -(alkylene)-R^{z} radical where R^{z} is heteroaryl as defined above, e.g., pyridinylmethyl, and the like. When the heteroaryl ring in heteroaralkyl contains 5- or 6 ring atoms it is also referred to herein as 5- or 6-membered heteroaralkyl.

“Heterocyclyl” means a saturated monovalent monocyclic radical of 4 to 8 ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —C(═O)— group. The heterocyclyl can be attached at any atom of the ring, valency permitting. More specifically the term heterocyclyl includes, but is not limited to, oxetanyl, pyrrolidinyl, piperidinyl, homopiperidinyl, 2-oxopyrrolidinyl, 2-oxopiperidinyl, morpholinyl, piperazinyl, tetrahydropyranyl, thiomorpholinyl, and the like. When heterocyclyl ring as defined above, contains only —SO₂— in the ring, it is also referred to herein “cyclylsulfonyl” and is a subset of the heterocyclyl group.

“Heterocyclyloxy” means a —OR^{z} radical where R^{z} is heterocyclyl as defined above. Representative examples include, but are not limited to, oxetanyloxy, piperidinyloxy, pyrrolidinyloxy, tetrahydrofuranyloxy, and the like.

“Heterocyclylalkyl” means a -(alkylene)-R^{z} radical where R^{z} is heterocyclyl as defined above. Representative examples include, but are not limited to, oxetanylmethyl, piperidinylmethyl, pyrrolidinylethyl, tetrahydrofuranylmethyl, and the like.

“Heterocyclylalkyloxy” means a —OR^{z} radical where R^{z} is heterocyclylalkyl as defined above. Representative examples include, but are not limited to, oxetanylmethyloxy, piperidinylmethyloxy, pyrrolidinylethyloxy, tetrahydrofuanylmethyloxy, and the like.

“Heterocyclyl^{A}” means a saturated monovalent monocyclic radical of 4 to 8 ring atoms in which one or two ring atoms are heteroatom independently selected from N and O, the remaining ring atoms being C, unless stated otherwise. Additionally, one or two ring carbon atoms in the heterocyclyl ring can optionally be replaced by a —C(═O)— group. More specifically the term heterocyclyl includes, but is not limited to, pyrrolidinyl, piperidinyl, homopiperidinyl, 2-oxopyrrolidinyl, 2-oxopiperidinyl, morpholinyl, piperazinyl, tetrahydropyranyl, and the like.

The term “oxo,” as used herein, alone or in combination, refers to ═(O).

“Optionally substituted aryl” means aryl as defined above, that is optionally substituted with one, two, or three substituents independently selected from alkyl, hydroxyl, cycloalkyl, carboxy, alkoxycarbonyl, hydroxy, alkoxy, alkylthio, alkylsulfonyl, amino, alkylamino, dialkylamino, halo, haloalkyl, haloalkoxy, and cyano.

“Optionally substituted aralkyl” means -(alkylene)-R^{z} where R^{z} is optionally substituted aryl as defined above.

“Optionally substituted heteroaryl” means heteroaryl as defined above that is optionally substituted with one, two, or three substituents independently selected from alkyl, alkylthio, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heteroaralkyl” means -(alkylene)-R^{z} where R^{z} is optionally substituted heteroaryl as defined above.

“Optionally substituted heterocyclyl” means heterocyclyl as defined above that is optionally substituted with one, two, or three substituents independently selected from alkyl, alkylthio, alkylsulfonyl, alkylcarbonyl, hydroxyl, cycloalkyl, cycloalkylalkyl, carboxy, alkoxycarbonyl, hydroxy, hydroxyalkyl, alkoxy, alkoxyalkyl, aminoalkyl, cyanoalkyl, halo, haloalkyl, haloalkoxy, and cyano, unless stated otherwise.

“Optionally substituted heterocyclylalkyl” means -(alkylene)-R^{z} where R^{z} is optionally substituted heterocyclyl as defined above.

“Substituted amino” means a —NR^{z}′R^{z}″ radical where R^{z}′ is hydrogen, alkyl, cycloalkyl, cycloalkylalkyl, haloalkyl, hydroxyalkyl, alkoxyalkyl, aminoalkyl, or alkylcarbonyl and R^{z}″ is cycloalkyl, cycloalkylalkyl, haloalkyl, hydroxyalkyl, alkoxyalkyl, aminoalkyl, alkylcarbonyl, optionally substituted aryl, optionally substituted aralkyl, optionally substituted heteroaryl, optionally substituted heteroaralkyl, optionally substituted heterocyclyl, or optionally substituted heterocyclylalkyl, each as defined herein. Representative examples include, but are not limited to, cyclopropylmethylamino, 2-(methylamino)ethylamino, hydroxyethylamino, and the like. When R^{z}′ is H and R^{z}″ is other than hydrogen, amino may also be referred to herein as monosubstituted amino. When R^{z}′ and R^{z}″ are other than hydrogen simultaneously, the term substituted amino may also be referred to herein as disubstituted amino.

“Spiroaminyl” means a saturated monovalent bicyclic ring of 6 to 12 ring atoms in which one ring atom is nitrogen and an additional ring atom can be a heteroatom independently selected from N, O, and S(O)ₙ, where n is an integer from 0 to 2, the remaining ring atoms being C, and further wherein the two rings are connected through only one atom, the connecting atom is also called the spiroatom, most often a quaternary carbon (“spiro carbon”). Representative examples of spiro cycloaminyl include, 2-azaspiro[3.3]heptan-2-yl, S-azaspiro[2.4]heptan-5-yl, 3-azaspiro[5.5]undecan-3-yl, 8-azaspiro[4.5]decan-8-yl, 6-azaspiro[3.4]octan-6-yl, 2-azaspiro[4.4]nonan-2-yl, but is not limited to, and the like.

“Spiro cycloalkyl” means a saturated bicyclic monovalent hydrocarbon ring having 7 to 11 ring atoms wherein the rings are connected through only one carbon atom, the connecting atom is also called the spiroatom, most often a quaternary carbon (“spiro carbon”). Spiro cycloalkyl is optionally substituted with one or two substituents independently selected from alkyl, halo, alkoxy, hydroxy, and cyano, unless stated otherwise. Representative examples include, but are not limited to, spiro[3,3]hept-2-yl, spiro[3,5]nonan-2-yl, and the like.

“Spiro heterocyclyl” means a saturated bicyclic monovalent ring having 6 to 10 ring atoms in which one, two, or three ring atoms are heteroatom selected from N, O, and S(O)n, where n is an integer selected from 0 to 2 inclusive, the remaining ring atoms being C and the rings are connected through only one atom, the connecting atom is also called the spiroatom, most often a quaternary carbon (“spiro carbon”). Spiroheterocyclyl is optionally substituted with one or two substituents independently selected from alkyl, halo, alkoxy, hydroxy, and cyano, unless otherwise stated. When spiro heterocyclyl contains only S(O)ₙ, it may be referred to herein as spiro cyclylsulfonyl. Representative examples include, but are not limited to, 2,6-diazaspiro-[3.3]heptanyl, 2,2-dioxido-2-thiaspiro[3.3]heptan-6-yl, 2,6-diazaspiro[3.4]octanyl, 2-azaspiro[3.4]octanyl, 2-azaspiro[3.5]-nonanyl, 2,7-diazaspiro[4.4]nonanyl, and the like.

When spiro heterocyclyl ring as defined above, contains only —SO₂— in the ring, it is also referred to herein “spiro sulfonyl” and is a subset of the heterocyclyl group.

“Spiro heterocyclylalkyl” means-(alkylene)-R radical where R is spiro heterocyclyl as defined above.

“Spiro heterocyclyl^{A}” means a saturated bicyclic monovalent ring having 6 to 10 ring atoms in which one or two ring atoms are heteroatom selected from N and O, the remaining ring atoms being C and the rings are connected through only one atom, the connecting atom is also called the spiroatom, most often a quaternary carbon (“spiro carbon”). Spiroheterocyclyl^{A} is optionally substituted with one or two substituents independently selected from alkyl, halo, alkoxy, hydroxy, and cyano, unless otherwise stated. Representative examples include, but are not limited to, 2,6-diazaspiro-[3.3]heptanyl, 2,6-diazaspiro[3.4]octanyl, 2-azaspiro[3.4]octanyl, 2-azaspiro[3.5]-nonanyl, 2,7-diazaspiro[4.4]nonanyl, and the like.

“Spiro sulfoximinyl” means a saturated monovalent bicyclic ring of 6 to 12 ring atoms in which one ring atom is >S(═O)(NR^{z}) where R^{z} is hydrogen or alkyl, as defined herein, the remaining ring atoms being C, and further wherein the two rings are connected through only one atom, the connecting atom is also called the spiroatom, most often a quaternary carbon (“spiro carbon”). Representative examples of spiro sulfoximinyl include, but is not limited to, 2-imino-2-oxo-2×6-thiaspiro[3.3]hepta-6-yl, 2-(2-methylimino)-2-oxo-2λ⁶-thiaspiro[3.3]hepta-6-yl, and the like.

“Unsaturated heterocyclyl” means a nonaromatic monovalent monocyclic radical of 5 to 8 ring atoms containing one or two double bonds and in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —C(═O)— group. The heterocyclyl can be attached at any atom of the ring, valency permitting. More specifically the term heterocyclyl includes, but is not limited to, dihydropyrrolidinyl, 1,2,3,6-tetrahydropyridine, 1,2-dihydropyridinyl, pyridin-2(1H)-one, and the like.

The present disclosure also includes protected derivatives of compounds of Formula (I). For example, when compounds of Formula (I) contain groups such as hydroxy, carboxy, or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of Formula (I) or a pharmaceutically acceptable salt thereof.

The term “prodrug” refers to a compound that is made more active in vivo. Certain compounds of Formula (I) may also exist as prodrugs, as described in Hydrolysis in Drug and Prodrug Metabolism: Chemistry, Biochemistry, and Enzymology (see Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference in its entirety.

The compounds of Formula (I) may have asymmetric centers. Compounds of Formula (I) containing an asymmetrically substituted atom may be isolated in optically active or racemic forms. Individual stereoisomers of compounds can be synthesized from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diasteromeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding(S) stereoisomer as an impurity and vice versa.

Certain compounds of Formula (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formula (I) are within the scope of this disclosure.

The compounds of Formula (I) may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (I) (and any embodiments thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds of Formula (I), including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

Certain structures, including Formula (I), provided herein are drawn with one or more floating substituents i.e., in the structure below, R¹, R², R³ and R⁶ are floating substituents. Unless provided otherwise or otherwise clear from the context, the substituent(s) may be present on any atom of the ring to which it is attached, including any ring atom denoted as CH of such ring unless stated otherwise, where chemically feasible and valency rules permitting. For example, in the structure:

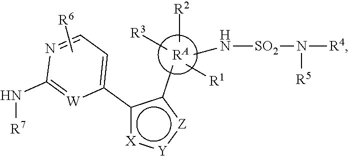

the R⁶ substituent can replace any hydrogen on the azabenzo ring, including the hydrogen of CH when W is CH.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase “optionally” or “optional” as used herein means that the subsequently described event or circumstance may but need not occur, and that the description includes instances where the event or circumstance occurs and instances in which it does not. For example, the phrase “cycloalkyl optionally substituted with alkyl” is intended to cover cycloalkyl that is unsubstituted with alkyl and cycloalkyl that is substituted with alkyl.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., delaying, arresting (i.e., stabilizing), or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated.

The terms “inhibiting” and “reducing,” or any variation of these terms in relation to CDK2, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of CDK2 activity compared to its normal activity.

Representative compounds of the disclosure made are disclosed in Table 1 below:

TABLE 1

| Cpd | | | [M + |
|---|---|---|---|
| # | Structure | Name | H]⁺ |
| 1 | 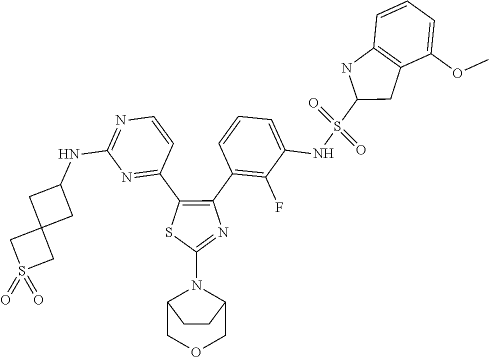 [SMILES: COc1cccc2c1CC(S(=O)(=O)Nc1cccc(-c3nc(N4C5CCC4COC5)sc3-c3ccnc(NC4CC5(C4)CS(=O)(=O)C5)n3)c1F)N2] | N-(3-(2-((1R,5S)- 3-oxa-8-aza- bicyclo[3.2.1]-octan- 8-yl)-5-(2-((2,2- dioxido-2-thiaspiro- [3.3]heptan-6- yl)amino)pyrimidin-4- yl)thiazol-4- yl)-2- fluorophenyl)-4- methoxyisoindoline-2- sulfonamide | 754.2 |

| | | | |
|---|---|---|---|
| 2 | 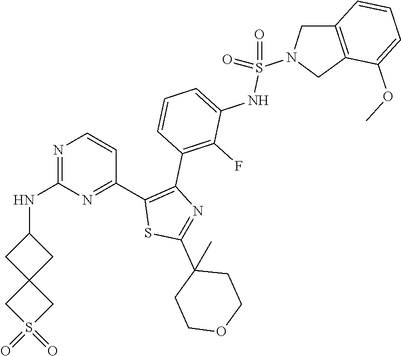 [SMILES: COc1cccc2c1CN(S(=O)(=O)Nc1cccc(-c3nc(C4(C)CCOCC4)sc3-c3ccnc(NC4CC5(C4)CS(=O)(=O)C5)n3)c1F)C2] | N-(3-(5-(2-((2,2- dioxido-2-thiaspiro- [3.3]-heptan-6-yl) amino)pyrimidin-4- yl)-2-(4- methyltetrahydro- 2H-pyran-4- yl)-thiazol-4- yl)-2-fluorophenyl)-4- methoxyisoindoline- 2-sulfonamide | 741.2 |

| | | | |
|---|---|---|---|
| 3 | 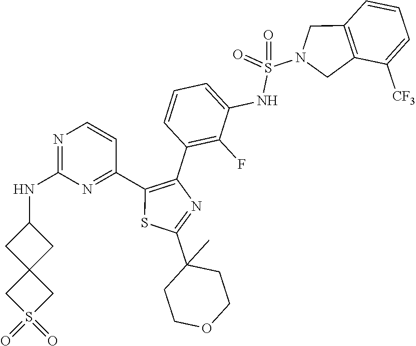 [SMILES: CC1(c2nc(-c3cccc(NS(=O)(=O)N4Cc5cccc(C(F)(F)F)c5C4)c3F)c(-c3ccnc(NC4CC5(C4)CS(=O)(=O)C5)n3)s2)CCOCC1] | N-(3-(5-(2-((2,2- dioxido-2-thiaspiro- [3.3]-heptan-6-yl) amino)pyrimidin-4- yl)-2-(4- methyltetrahydro- 2H-pyran-4- yl)thiazol-4- yl)-2-fluorophenyl)-4- (trifluoromethyl) isoindoline-2- sulfonamide | 779.2 |

| | | | |
|---|---|---|---|
| 4 | 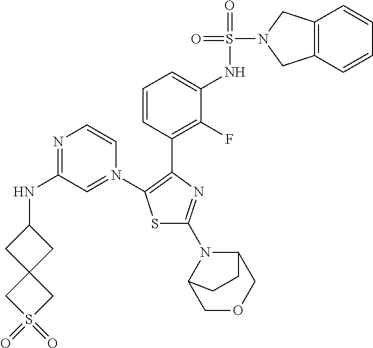 | N-(3-(2-(3-oxa-8- azabicyclo[3.2.1]octan- 8-yl)-5-(2-((2,2- dioxido-2-thiaspiro- [3.3]heptan-6-yl) amino)pyrimidin-4- yl)thiazol-4- yl)-2-fluorophenyl)- isoindoline-2-sulfonamide | 724.2 |

| | | | |
|---|---|---|---|
| 5 | 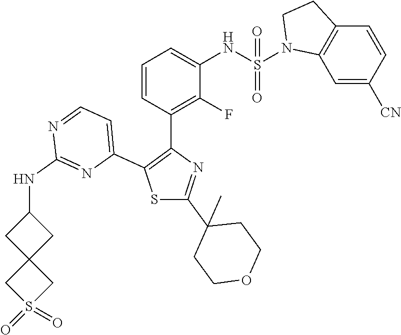 [SMILES: CC1(c2nc(-c3cccc(NS(=O)(=O)N4CCc5ccc(C#N)cc54)c3F)c(-c3ccnc(NC4CC5(C4)CS(=O)(=O)C5)n3)s2)CCOCC1] | 6-cyano-N-(3-(5- (2-((2,2-dioxido-2- thiaspiro-[3.3]heptan- 6-yl)amino)- pyrimidin-4- yl)-2-(4- methyltetrahydro- 2H-pyran-4- yl)thiazol-4- yl)-2- fluorophenyl) indoline- 1-sulfonamide | 736.2 |

| | | | |
|---|---|---|---|
| 6 | 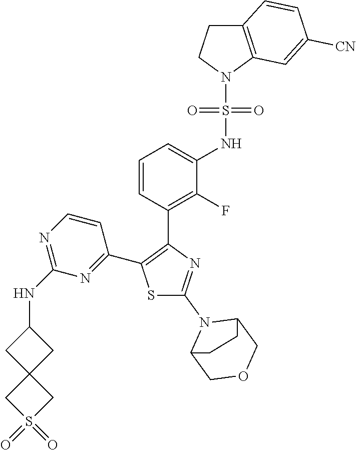 [SMILES: N#Cc1ccc2c(c1)N(S(=O)(=O)Nc1cccc(-c3nc(N4C5CCC4COC5)sc3-c3ccnc(NC4CC5(C4)CS(=O)(=O)C5)n3)c1F)CC2] | N-(3-(2-(3-oxa-8- azabicyclo[3.2.1]octan- 8-yl)-5-(2-((2,2- dioxido-2-thiaspiro- [3.3]heptan-6-yl) amino)pyrimidin-4- yl)thiazol-4- yl)-2-fluorophenyl)- 6-cyanoindoline- 1-sulfonamide | 749.2 |

| | | | |
|---|---|---|---|
| 7 | 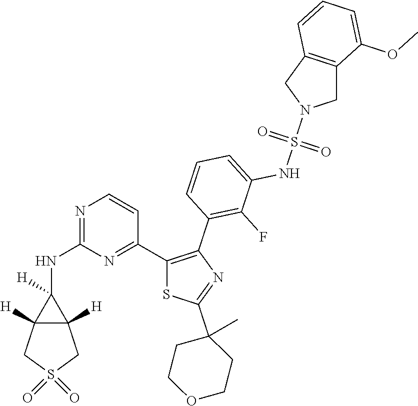 [SMILES: COc1cccc2c1CN(S(=O)(=O)Nc1cccc(-c3nc(C4(C)CCOCC4)sc3-c3ccnc(N[C@H]4[C@@H]5CS(=O)(=O)C[C@@H]54)n3)c1F)C2] | N-(3-(5-(2-(((1R, 5S,6r)-3,3-dioxido- 3-thiabicyclo[3.1.0] hexan-6-yl)amino)- pyrimidin-4- yl)-2-(4- methyltetrahydro- 2H-pyran-4- yl)thiazol-4- yl)-2- fluorophenyl)-4- methoxyisoindoline- 2-sulfonamide | 727.2 |

| | | | |
|---|---|---|---|
| 8 | 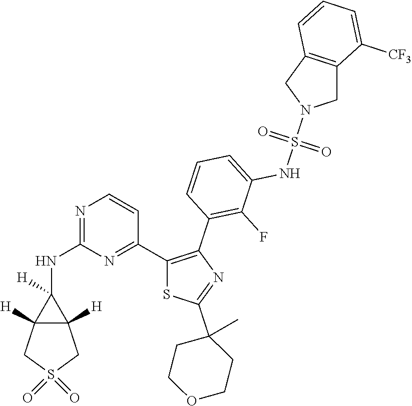 [SMILES: CC1(c2nc(-c3cccc(NS(=O)(=O)N4Cc5cccc(C(F)(F)F)c5C4)c3F)c(-c3ccnc(N[C@H]4[C@@H]5CS(=O)(=O)C[C@@H]54)n3)s2)CCOCC1] | N-(3-(5-(2-(((1R,5S, 6r)-3,3-dioxido-3- thiabicyclo[3.1.0] hexan-6-yl)amino)- pyrimidin-4- yl)-2-(4- methyltetrahydro- 2H-pyran-4- yl)thiazol-4- yl)-2- fluorophenyl)-4- (trifluoromethyl)- isoindoline-2- sulfonamide | 765.2 |
| 9 | 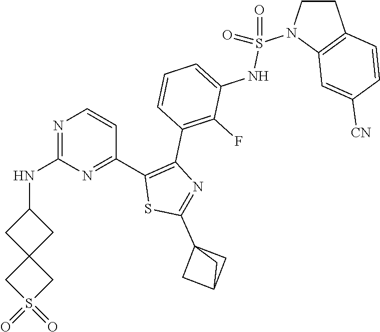 [SMILES: N#Cc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 6-cyanoindoline- 1-sulfonamide | 704.3 |
| 10 | 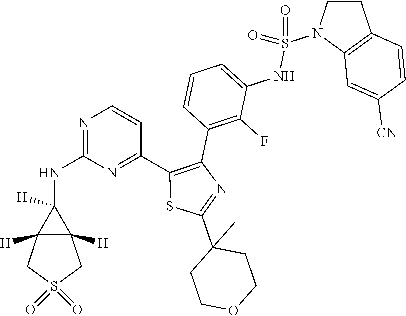 [SMILES: CC1(c2nc(-c3cccc(NS(=O)(=O)N4CCc5ccc(C#N)cc54)c3F)c(-c3ccnc(N[C@H]4[C@@H]5CS(=O)(=O)C[C@@H]54)n3)s2)CCOCC1] | 6-cyano-N-(3-(5-(2- (((1R,5S,6r)-3,3- dioxido-3-thiabicyclo [3.1.0]hexan-6- yl)amino)-pyrimidin- 4-yl)-2-(4- methyltetrahydro- 2H-pyran-4- yl)thiazol-4- yl)-2-fluorophenyl)- indoline- 1-sulfonamide | 722.3 |
| 11 | 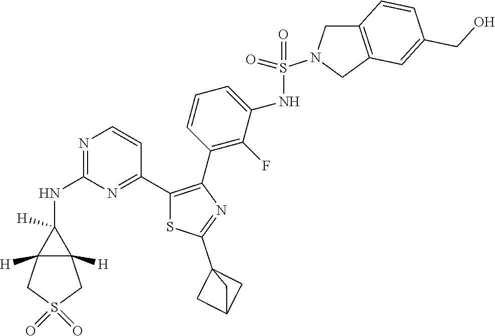 [SMILES: O=S1(=O)C[C@@H]2[C@H](C1)[C@H]2Nc1nccc(-c2sc(C34CC(C3)C4)nc2-c2cccc(NS(=O)(=O)N3Cc4ccc(CO)cc4C3)c2F)n1] | N-(3-(2-(bicyclo [1.1.1]pentan- 1-yl)-5-(2- (((1R,5S,6r)-3,3- dioxido-3- thiabicyclo- [3.1.0]hexan-6-yl) amino)pyrimidin-4- yl)thiazol-4- yl)-2-fluorophenyl)- 5- (hydroxymethyl) isoindoline-2- sulfonamide | 695.2 |
| 12 | 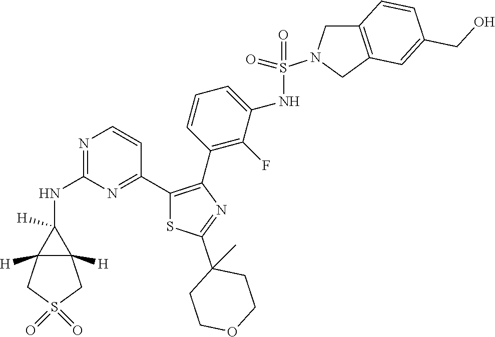 [SMILES: CC1(c2nc(-c3cccc(NS(=O)(=O)N4Cc5ccc(CO)cc5C4)c3F)c(-c3ccnc(N[C@H]4[C@@H]5CS(=O)(=O)C[C@@H]54)n3)s2)CCOCC1] | N-(3-(5-(2-(((1R, 5S,6r)-3,3-dioxido-3- thiabicyclo[3.1.0] hexan-6-yl)amino)- pyrimidin-4- yl)-2-(4- methyltetrahydro- 2H-pyran-4- yl)thiazol-4- yl)-2- fluorophenyl)-5- (hydroxymethyl)- isoindoline-2- sulfonamide | 727.3 |
| 13 | 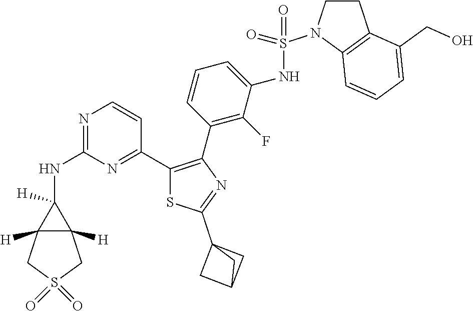 [SMILES: O=S1(=O)C[C@@H]2[C@H](C1)[C@H]2Nc1nccc(-c2sc(C34CC(C3)C4)nc2-c2cccc(NS(=O)(=O)N3CCc4c(CO)cccc43)c2F)n1] | N-(3-(2-(bicyclo [1.1.1]pentan- 1-yl)-5-(2- (((1R,5S,6r)-3,3- dioxido-3-thia- bicyclo[3.1.0]- hexan-6-yl)amino)- pyrimidin-4- yl)thiazol-4- yl)-2- fluorophenyl)-4- (hydroxymethyl)- indoline- 1-sulfonamide | 695.3 |
| 14 | 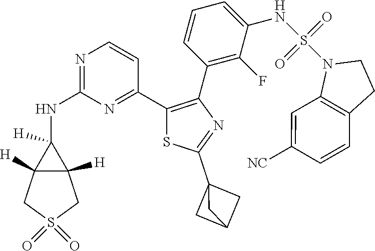 [SMILES: N#Cc1ccc2c(c1)N(S(=O)(=O)Nc1cccc(-c3nc(C45CC(C4)C5)sc3-c3ccnc(N[C@H]4[C@@H]5CS(=O)(=O)C[C@@H]54)n3)c1F)CC2] | N-(3-(2-(bicyclo [1.1.1]pentan- 1-yl)-5-(2- (((1R,5S,6r)-3,3- dioxido-3- thiabicyclo- [3.1.0]-hexan-6-yl) amino)pyrimidin-4- yl)thiazol-4- yl)-2-fluorophenyl)- 6-cyanoindoline- 1-sulfonamide | 690.2 |
| 15 | 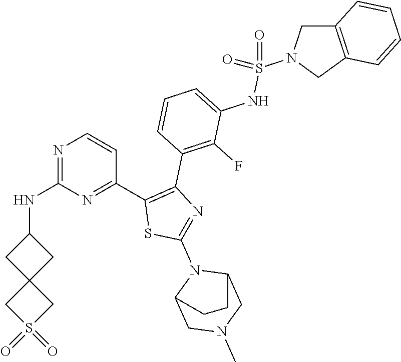 [SMILES: CN1CC2CCC(C1)N2c1nc(-c2cccc(NS(=O)(=O)N3Cc4ccccc4C3)c2F)c(-c2ccnc(NC3CC4(C3)CS(=O)(=O)C4)n2)s1] | N-(3-(5-(2-((2,2- dioxido-2-thiaspiro- [3.3]-heptan-6-yl) amino)pyrimidin-4- yl)-2-(3- methyl-3,8- diazabicyclo[3.2.1]- octan-8-yl)thiazol-4- yl)-2-fluorophenyl)- isoindoline-2- sulfonamide | 737.2 |
| 16 | 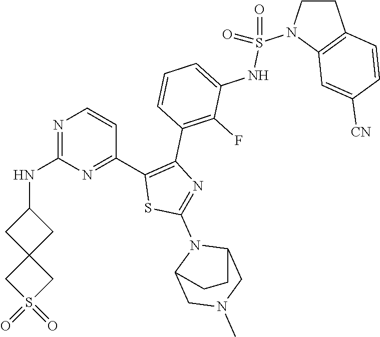 [SMILES: CN1CC2CCC(C1)N2c1nc(-c2cccc(NS(=O)(=O)N3CCc4ccc(C#N)cc43)c2F)c(-c2ccnc(NC3CC4(C3)CS(=O)(=O)C4)n2)s1] | 6-cyano-N-(3-(5- (2-((2,2-dioxido-2- thiaspiro[3.3] heptan-6-yl)amino)- pyrimidin-4- yl)-2-(3- methyl-3,8- diazabicyclo[3.2.1] octan-8-yl)thiazol-4- yl)-2-fluorophenyl) indoline-1- sulfonamide | 762.3 |
| 17 | 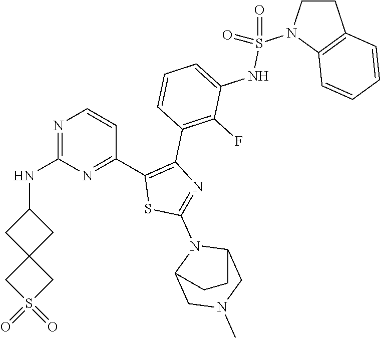 [SMILES: CN1CC2CCC(C1)N2c1nc(-c2cccc(NS(=O)(=O)N3CCc4ccccc43)c2F)c(-c2ccnc(NC3CC4(C3)CS(=O)(=O)C4)n2)s1] | N-(3-(5-(2-((2,2- dioxido-2-thiaspiro- [3.3]-heptan-6-yl) amino)pyrimidin-4- yl)-2-(3- methyl-3,8- diazabicyclo[3.2.1]- octan-8-yl)thiazol-4- yl)-2-fluorophenyl)- indoline- 1-sulfonamide | 737.2 |
| 18 | 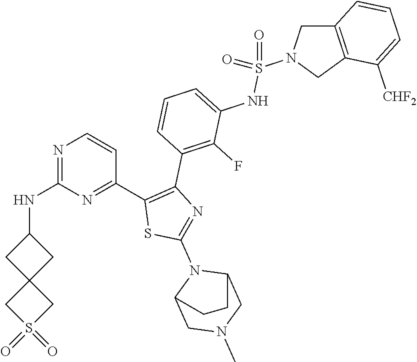 [SMILES: CN1CC2CCC(C1)N2c1nc(-c2cccc(NS(=O)(=O)N3Cc4cccc(C(F)F)c4C3)c2F)c(-c2ccnc(NC3CC4(C3)CS(=O)(=O)C4)n2)s1] | 4-(difluoromethyl)- N-(3-(5-(2-((2,2- dioxido-2-thiaspiro [3.3]heptan-6- yl)amino)pyrimidin- 4-yl)-2-(3- methyl- 3,8-diazabicyclo [3.2.1]octan-8- yl)thiazol-4- yl)-2-fluorophenyl)- isoindoline-2- sulfonamide | 787.2 |
| 19 | 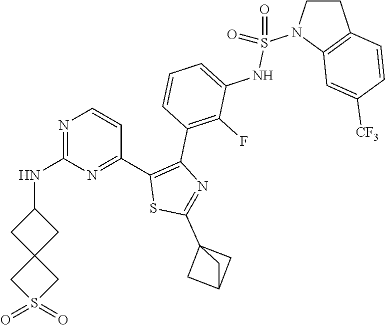 [SMILES: O=S1(=O)CC2(CC(Nc3nccc(-c4sc(C56CC(C5)C6)nc4-c4cccc(NS(=O)(=O)N5CCc6ccc(C(F)(F)F)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 6-(trifluoromethyl)- indoline- 1-sulfonamide | 747.3 |
| 20 | 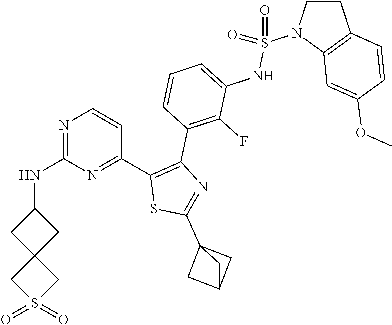 [SMILES: COc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-6- methoxyindoline- 1-sulfonamide | 709.2 |
| 21 | 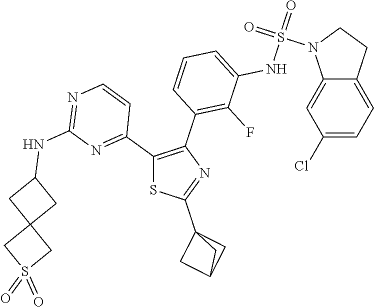 [SMILES: O=S1(=O)CC2(CC(Nc3nccc(-c4sc(C56CC(C5)C6)nc4-c4cccc(NS(=O)(=O)N5CCc6ccc(Cl)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 6-chloroindoline- 1-sulfonamide | 713.2 |
| 22 | 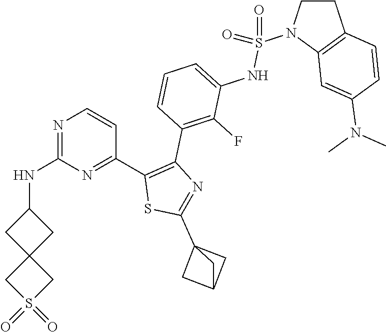 [SMILES: CN(C)c1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | N-(3-(2-(bicyclo [1.1.1]pentan- 1-yl)-5-(2- ((2,2-dioxido- 2-thiaspiro [3.3]heptan-6- yl)amino)- pyrimidin-4- yl)thiazol-4- yl)-2- fluorophenyl)- 6-(dimethylamino)- indoline- 1-sulfonamide | 722.3 |
| 23 | 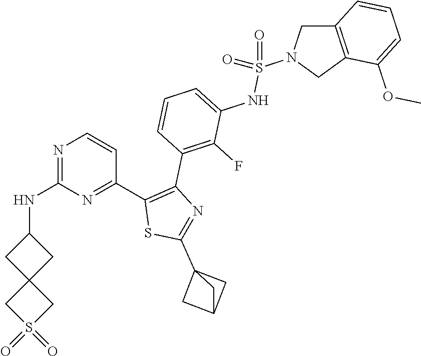 [SMILES: COc1cccc2c1CN(S(=O)(=O)Nc1cccc(-c3nc(C45CC(C4)C5)sc3-c3ccnc(NC4CC5(C4)CS(=O)(=O)C5)n3)c1F)C2] | N-(3-(2-(bicyclo [1.1.1]pentan- 1-yl)-5-(2- ((2,2-dioxido- 2-thiaspiro[3.3] heptan-6- yl)amino)- pyrimidin-4- yl)thiazol-4- yl)-2- fluorophenyl)-4- methoxyisoindoline-2- sulfonamide | 709.2 |
| 24 | 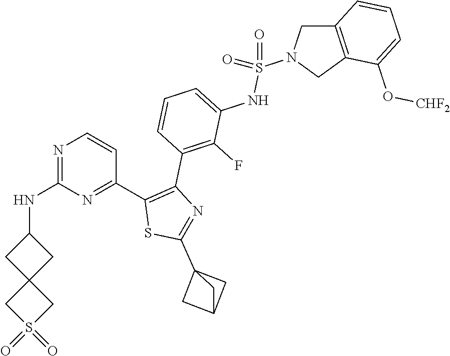 [SMILES: O=S1(=O)CC2(CC(Nc3nccc(-c4sc(C56CC(C5)C6)nc4-c4cccc(NS(=O)(=O)N5Cc6cccc(OC(F)F)c6C5)c4F)n3)C2)C1] | N-(3-(2~(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-4- (difluoromethoxy)- isoindoline-2- sulfonamide | 745.2 |
| 25 | 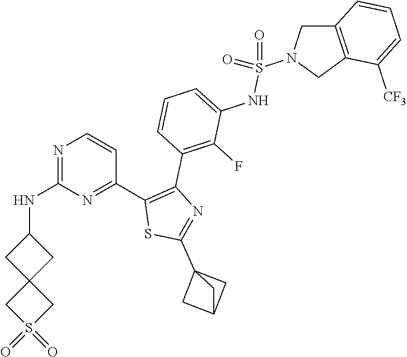 [SMILES: O=S1(=O)CC2(CC(Nc3nccc(-c4sc(C56CC(C5)C6)nc4-c4cccc(NS(=O)(=O)N5Cc6cccc(C(F)(F)F)c6C5)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-4- (trifluoromethyl)- isoindoline-2- sulfonamide | 747.2 |
| 26 | 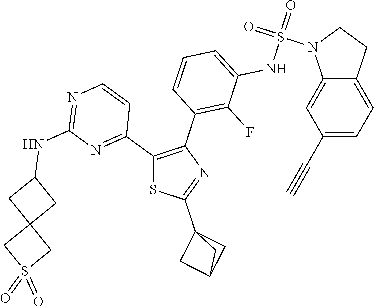 [SMILES: C#Cc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | N-(3-(2-(bicyclo [1.1.1]pentan- 1-yl)-5-(2- ((2,2-dioxido-2- thiaspiro[3.3]heptan- 6-yl)amino)- pyrimidin-4- yl)thiazol-4- yl)-2- fluorophenyl)-6- ethynylindoline- 1-sulfonamide | 703.3 |
| 27 | 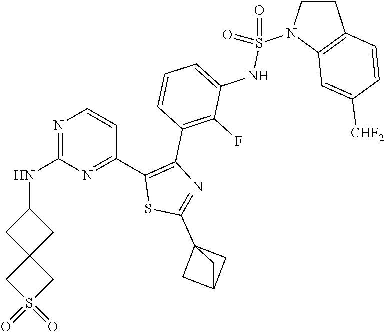 [SMILES: O=S1(=O)CC2(CC(Nc3nccc(-c4sc(C56CC(C5)C6)nc4-c4cccc(NS(=O)(=O)N5CCc6ccc(C(F)F)cc65)c4F)n3)C2)C1] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-6- (difluoromethyl)- indoline- 1-sulfonamide | 729.2 |
| 28 | 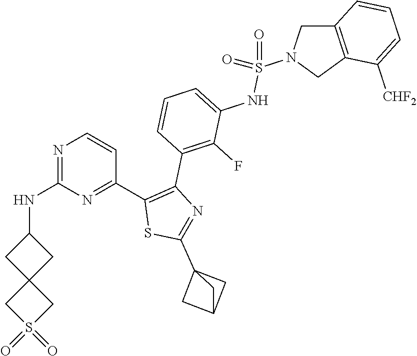 [SMILES: O=S1(=O)CC2(CC(Nc3nccc(-c4sc(C56CC(C5)C6)nc4-c4cccc(NS(=O)(=O)N5Cc6cccc(C(F)F)c6C5)c4F)n3)C2)C1] | N-(3-(2-(bicyclo [1.1.1]pentan- 1-yl)-5-(2- ((2,2-dioxido- 2-thiaspiro[3.3] heptan-6- yl)amino)-pyrimidin- 4-yl)thiazol-4- yl)-2- fluorophenyl)-4- (difluoromethyl)- isoindoline-2- sulfonamide | 729.2 |
| 29 | 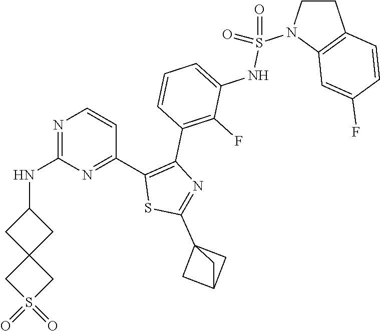 [SMILES: O=S1(=O)CC2(CC(Nc3nccc(-c4sc(C56CC(C5)C6)nc4-c4cccc(NS(=O)(=O)N5CCc6ccc(F)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 6-fluoroindoline- 1-sulfonamide | 697.2 |
| 30 | 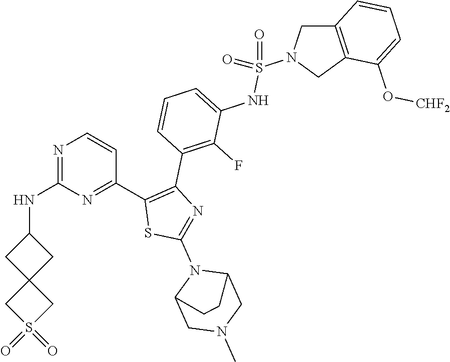 [SMILES: CN1CC2CCC(C1)N2c1nc(-c2cccc(NS(=O)(=O)N3Cc4cccc(OC(F)F)c4C3)c2F)c(-c2ccnc(NC3CC4(C3)CS(=O)(=O)C4)n2)s1] | 4-(difluoromethoxy)- N-(3-(5-(2-((2,2- dioxido-2-thiaspiro [3.3]heptan-6- yl)amino) pyrimidin-4- yl)-2-(3- methyl- 3,8-diazabicyclo [3.2.1]-octan-8- yl)thiazol-4- yl)-2-fluorophenyl)- isoindoline-2- sulfonamide | 803.3 |
| 31 | 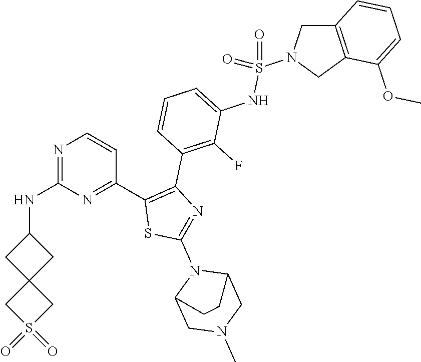 [SMILES: COc1cccc2c1CN(S(=O)(=O)Nc1cccc(-c3nc(N4C5CCC4CN(C)C5)sc3-c3ccnc(NC4CC5(C4)CS(=O)(=O)C5)n3)c1F)C2] | N-(3-(5-(2-((2,2- dioxido-2-thiaspiro- [3.3]-heptan-6-yl) amino)pyrimidin-4- yl)-2-(3- methyl-3,8- diazabicyclo [3.2.1]- octan-8-yl)- thiazol-4- yl)-2-fluoro- phenyl)-4- methoxy- isoindoline-2- sulfonamide | 767.3 |
| 32 | 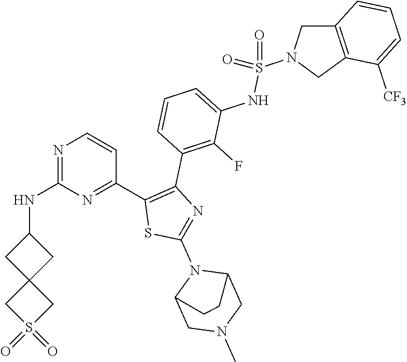 [SMILES: CN1CC2CCC(C1)N2c1nc(-c2cccc(NS(=O)(=O)N3Cc4cccc(C(F)(F)F)c4C3)c2F)c(-c2ccnc(NC3CC4(C3)CS(=O)(=O)C4)n2)s1] | N-(3-(5-(2-((2,2- dioxido-2-thiaspiro- [3.3]-heptan-6-yl) amino)pyrimidin-4- yl)- 2-(3- methyl-3,8- diazabicyclo[3.2.1]- octan-8-yl)-thiazol-4- yl)-2-fluoro- phenyl)-4- (trifluoro- methyl)isoindoline- 2-sulfonamide | 805.2 |
| 33 | 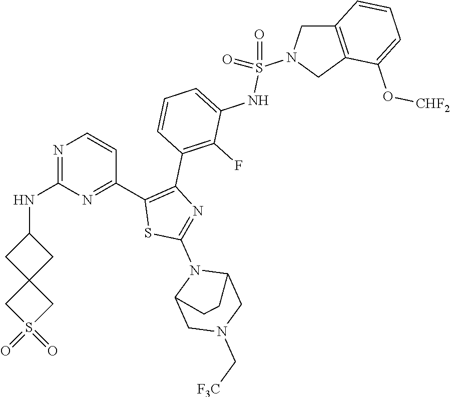 [SMILES: O=S1(=O)CC2(CC(Nc3nccc(-c4sc(N5C6CCC5CN(CC(F)(F)F)C6)nc4-c4cccc(NS(=O)(=O)N5Cc6cccc(OC(F)F)c6C5)c4F)n3)C2)C1] | 4-(difluoromethoxy)- N-(3-(5-(2-((2,2- dioxido-2-thiaspiro [3.3]heptan-6- yl)amino)pyrimidin-4- yl)-2-(3-(2,2,2- trifluoroethyl)-3, 8-diazabicyclo- [3.2.1]octan-8- yl)thiazol-4- yl)-2- fluorophenyl) isoindoline-2- sulfonamide | 871.3 |
| 34 | 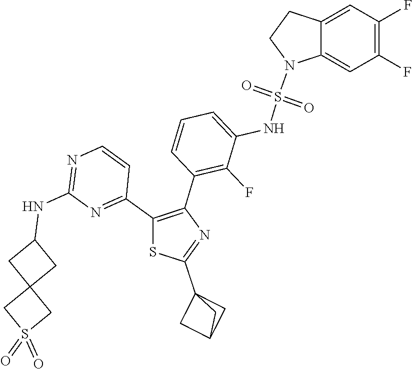 [SMILES: O=S1(=O)CC2(CC(Nc3nccc(-c4sc(C56CC(C5)C6)nc4-c4cccc(NS(=O)(=O)N5CCc6cc(F)c(F)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-5, 6-difluoroindoline-1- sulfonamide | 715.2 |
| 35 | 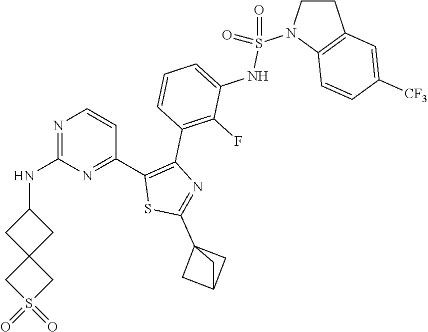 [SMILES: O=S1(=O)CC2(CC(Nc3nccc(-c4sc(C56CC(C5)C6)nc4-c4cccc(NS(=O)(=O)N5CCc6cc(C(F)(F)F)ccc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-5- (trifluoromethyl)- indoline- 1-sulfonamide | 747.2 |
| 36 | 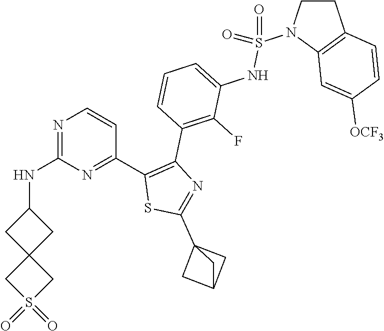 [SMILES: O=S1(=O)CC2(CC(Nc3nccc(-c4sc(C56CC(C5)C6)nc4-c4cccc(NS(=O)(=O)N5CCc6ccc(OC(F)(F)F)cc65)c4F)n3)C2)C1] | N-(3-(2-(bicyclo [1.1.1]pentan- 1-yl)-5-(2- ((2,2-dioxido- 2-thiaspiro [3.3]heptan-6- yl)amino)- pyrimidin-4- yl)thiazol-4- yl)-2- fluorophenyl)-6- (trifluoromethoxy) indoline-1- sulfonamide | 763.2 |
| 37 | 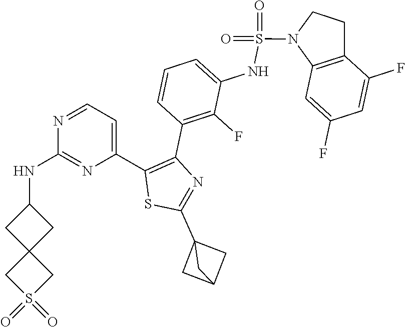 [SMILES: O=S1(=O)CC2(CC(Nc3nccc(-c4sc(C56CC(C5)C6)nc4-c4cccc(NS(=O)(=O)N5CCc6c(F)cc(F)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-4, 6-difluoroindoline- 1-sulfonamide | 715.1 |
| 38 | 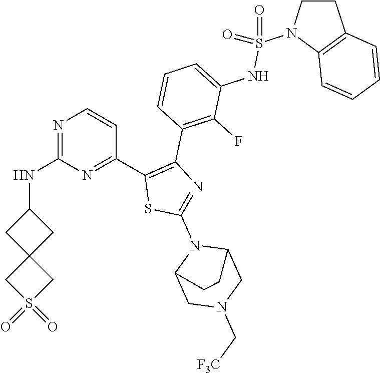 [SMILES: O=S1(=O)CC2(CC(Nc3nccc(-c4sc(N5C6CCC5CN(CC(F)(F)F)C6)nc4-c4cccc(NS(=O)(=O)N5CCc6ccccc65)c4F)n3)C2)C1] | N-(3-(5-(2-((2,2- dioxido-2-thiaspiro- [3.3]-heptan-6-yl) amino)pyrimidin-4- yl)- 2-(3-(2,2,2- trifluoroethyl)- 3,8- diazabicyclo[3.2.1]- octan-8-yl)thiazol-4- yl)-2-fluorophenyl)- indoline-1- sulfonamide | 805.3 |
| 39 | 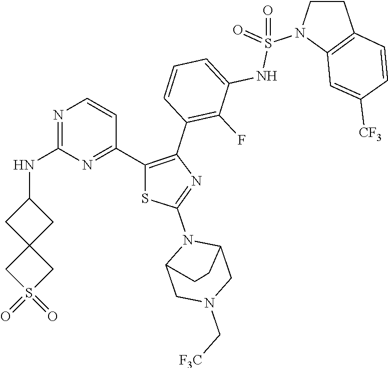 [SMILES: O=S1(=O)CC2(CC(Nc3nccc(-c4sc(N5C6CCC5CN(CC(F)(F)F)C6)nc4-c4cccc(NS(=O)(=O)N5CCc6ccc(C(F)(F)F)cc65)c4F)n3)C2)C1] | N-(3-(5-(2-((2,2- dioxido-2-thiaspiro- [3.3]-heptan-6-yl) amino)pyrimidin-4- yl)- 2-(3-(2,2,2- trifluoroethyl)-3,8- diazabicyclo[3.2.1]- octan-8-yl)thiazol-4- yl)-2-fluorophenyl)- 6-(trifluoromethyl)- indoline- 1-sulfonamide | 873.3 |
| 40 | 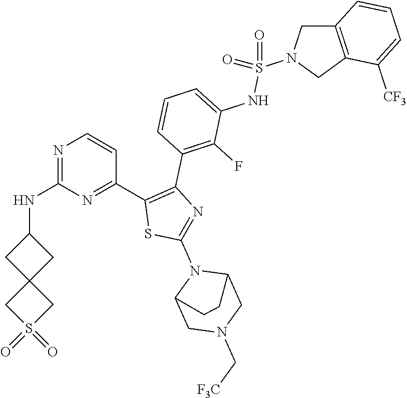 [SMILES: O=S1(=O)CC2(CC(Nc3nccc(-c4sc(N5C6CCC5CN(CC(F)(F)F)C6)nc4-c4cccc(NS(=O)(=O)N5Cc6cccc(C(F)(F)F)c6C5)c4F)n3)C2)C1] | N-(3-(5-(2-((2,2- dioxido-2-thiaspiro- [3.3]-heptan-6-yl) amino)pyrimidin-4- yl)-2-(3-(2,2,2- trifluoroethyl)-3,8- diazabicyclo[3.2.1]- octan-8-yl)thiazol-4- yl)-2- fluorophenyl)-4- (trifluoromethyl)- isoindoline-2-sulfonamide | 873.2 |
| 41 | 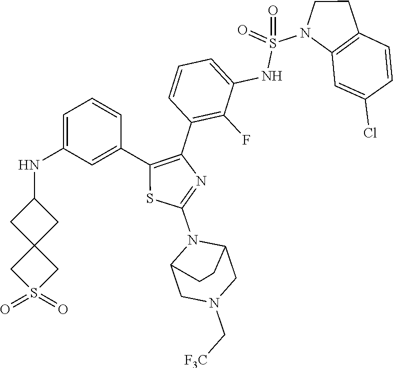 [SMILES: O=S1(=O)CC2(CC(Nc3cccc(-c4sc(N5C6CCC5CN(CC(F)(F)F)C6)nc4-c4cccc(NS(=O)(=O)N5CCc6ccc(Cl)cc65)c4F)c3)C2)C1] | 6-chloro-N-(3-(5- (2-((2,2-dioxido-2- thiaspiro[3.3] heptan-6- yl)amino)- pyrimidin-4- yl)-2-(3-(2,2,2- trifluoroethyl)-3, 8-diazabicyclo [3.2.1]- octan-8-yl) thiazol-4- yl)-2-fluoro- phenyl)indoline- 1-sulfonamide | 839.2 |
| 42 | 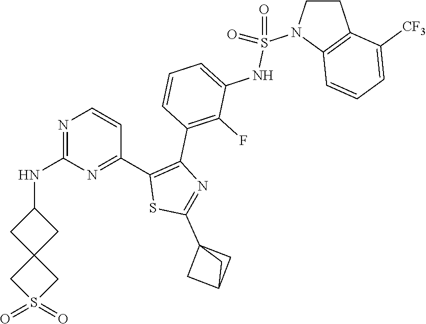 [SMILES: O=S1(=O)CC2(CC(Nc3nccc(-c4sc(C56CC(C5)C6)nc4-c4cccc(NS(=O)(=O)N5CCc6c5cccc6C(F)(F)F)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-4- (trifluoromethyl)- indoline- 1-sulfonamide | 747.2 |
| 43 | 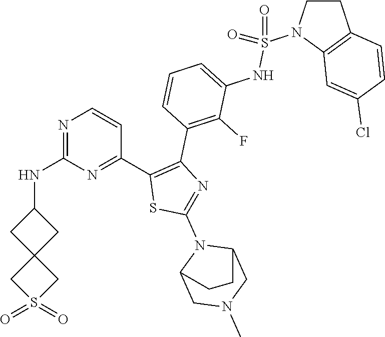 [SMILES: CN1CC2CCC(C1)N2c1nc(-c2cccc(NS(=O)(=O)N3CCc4ccc(Cl)cc43)c2F)c(-c2ccnc(NC3CC4(C3)CS(=O)(=O)C4)n2)s1] | 6-chloro-N-(3-(5- (2-((2,2-dioxido-2- thiaspiro[3.3] heptan-6- yl)amino)- pyrimidin-4- yl)-2-(3- methyl-3,8- diazabicyclo [3.2.1]octan-8- yl)thiazol-4- yl)-2- fluorophenyl) indoline-1- sulfonamide | 771.3 |
| 44 | 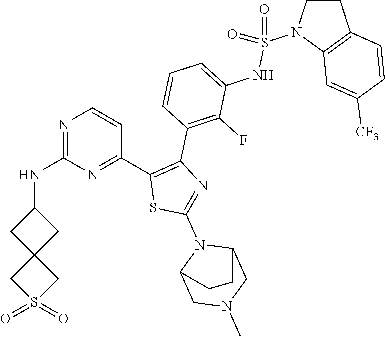 [SMILES: CN1CC2CCC(C1)N2c1nc(-c2cccc(NS(=O)(=O)N3CCc4ccc(C(F)(F)F)cc43)c2F)c(-c2ccnc(NC3CC4(C3)CS(=O)(=O)C4)n2)s1] | N-(3-(5-(2-((2,2- dioxido-2-thiaspiro- [3.3]heptan-6-yl) amino)pyrimidin-4- yl)-2-(3- methyl-3,8- diazabicyclo[3.2.1]- octan-8-yl)thiazol-4- yl)-2-fluorophenyl)- 6-(trifluoromethyl) indoline-1- sulfonamide | 805.3 |
| 45 | 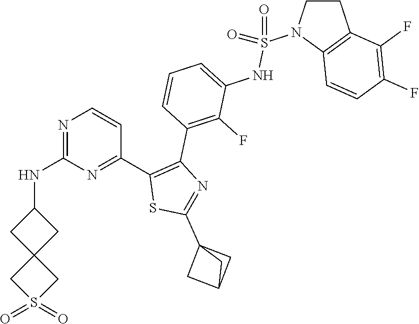 [SMILES: O=S1(=O)CC2(CC(Nc3nccc(-c4sc(C56CC(C5)C6)nc4-c4cccc(NS(=O)(=O)N5CCc6c5ccc(F)c6F)c4F)n3)C2)C1] | N-(3-(2-(bicyclo [1.1.1 ]pentan- 1-yl)-5-(2- ((2,2-dioxido-2- thiaspiro[3.3] heptan-6- yl)amino)- pyrimidin-4- yl)thiazol-4- yl)-2- fluorophenyl)- 4,5-difluoroindoline- 1- sulfonamide | 715.3 |
| 46 | 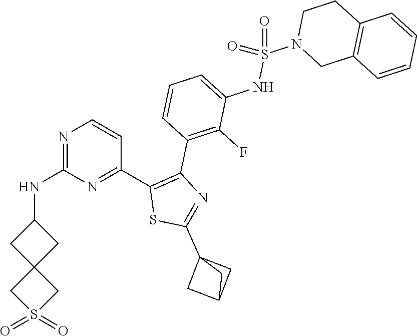 [SMILES: O=S1(=O)CC2(CC(Nc3nccc(-c4sc(C56CC(C5)C6)nc4-c4cccc(NS(=O)(=O)N5CCc6ccccc6C5)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 3,4- dihydroisoquinoline- 2(1H)-sulfonamide | 693.3 |
| 47 | 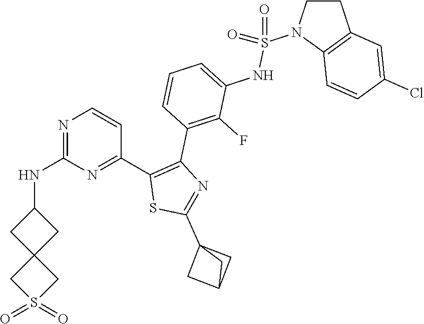 [SMILES: O=S1(=O)CC2(CC(Nc3nccc(-c4sc(C56CC(C5)C6)nc4-c4cccc(NS(=O)(=O)N5CCc6cc(Cl)ccc65)c4F)n3)C2)C1] | N-(3-(2-(bicyclo [1.1.1]pentan- 1-yl)-5-(2- ((2,2- dioxido-2- thiaspiro[3.3] heptan-6- yl)amino)- pyrimidin-4- yl)thiazol-4- yl)-2- fluorophenyl)- 5-chloroindoline- 1-sulfonamide | 713.2 |
| 48 | 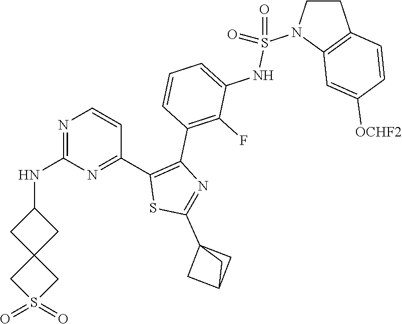 [SMILES: O=S1(=O)CC2(CC(Nc3nccc(-c4sc(C56CC(C5)C6)nc4-c4cccc(NS(=O)(=O)N5CCc6ccc(OC(F)F)cc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-6- (difluoromethoxy)- indoline- 1-sulfonamide | 745.3 |
| 49 | 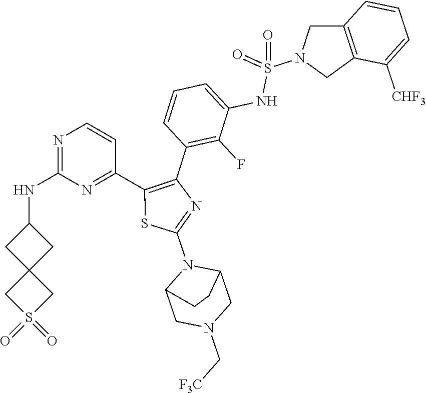 | 4-(difluoromethyl)- N-(3-(5-(2-((2,2- dioxido-2-thiaspiro [3.3]heptan-6- yl)amino) pyrimidin-4- yl)-2-(3-(2,2,2- trifluoroethyl)- 3,8-diazabicyclo [3.2.1]- octan-8-yl) thiazol-4- yl)-2-fluoro- phenyl)isoindoline- 2-sulfonamide | 855.3 |
| 50 | 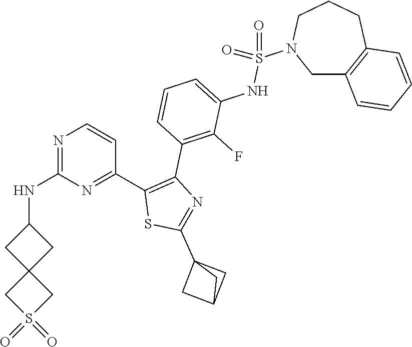 [SMILES: O=S1(=O)CC2(CC(Nc3nccc(-c4sc(C56CC(C5)C6)nc4-c4cccc(NS(=O)(=O)N5CCCc6ccccc6C5)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 1,3,4,5- tetrahydro-2H- benzo[c]azepine- 2-sulfonamide | 707.2 |
| 51 | 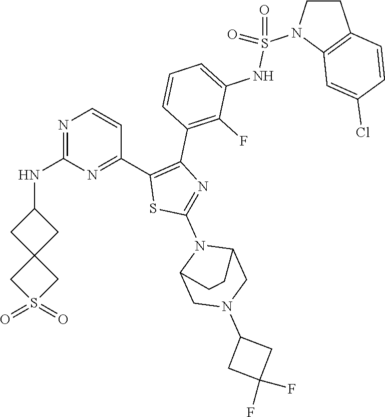 [SMILES: O=S1(=O)CC2(CC(Nc3nccc(-c4sc(N5C6CCC5CN(C5CC(F)(F)C5)C6)nc4-c4cccc(NS(=O)(=O)N5CCc6ccc(Cl)cc65)c4F)n3)C2)C1] | 6-chloro-N-(3- (2-(3-(3,3- difluoro- cyclobutyl)-3, 8-diazabicyclo [3.2.1 ]octan- 8-yl)-5-(2-((2,2- dioxido-2- thiaspiro- [3.3]heptan-6-yl) amino)- pyrimidin-4- yl)thiazol-4- yl)-2- fluorophenyl)- indoline- 1-sulfonamide | 847.2 |
| 52 | 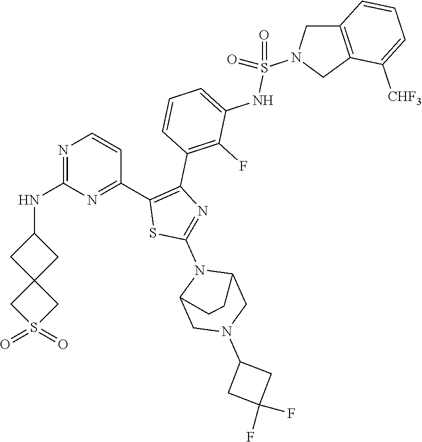 | N-(3-(2-(3-(3,3- difluorocyclobutyl)- 3,8- diazabicyclo [3.2.1]octan- 8-yl)-5-(2- ((2,2-dioxido-2- thiaspiro[3.3] heptan-6- yl)amino)- pyrimidin-4- yl)thiazol-4- yl)-2- fluorophenyl)-4- (difluoromethyl)- isoindoline-2- sulfonamide | 863.2 |
| 53 | 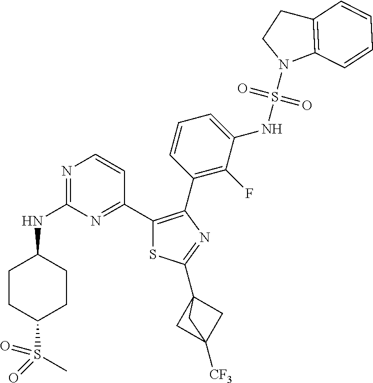 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccccc54)c3F)n2)CC1] | N-(2-fluoro-3-(5- (2-(((1r,4r)-4- (methyl- sulfonyl) cyclohexyl) amino) pyrimidin-4- yl)-2-(3- (trifluoromethyl) bicyclo- [1.1.1]pentan- 1-yl)thiazol-4- yl)phenyl)- indoline- 1-sulfonamide | 763.2 |
| 54 | 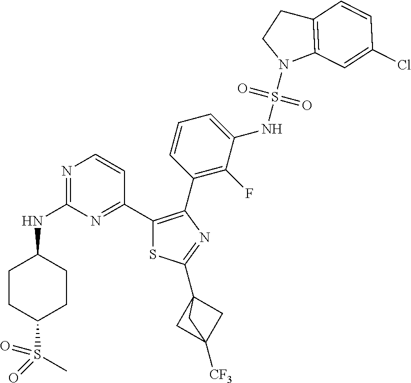 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(Cl)cc54)c3F)n2)CC1] | 6-chloro-N-(2- fluoro-3-(5-(2- (((1r,4r)-4- (methylsulfonyl) cyclohexyl) amino) pyrimidin-4- yl)-2-(3- (trifluoromethyl) bicyclo- [1.1.1]-pentan- 1-yl)thiazol-4- yl)phenyl)- indoline- 1-sulfonamide | 796.9 |
| 55 | 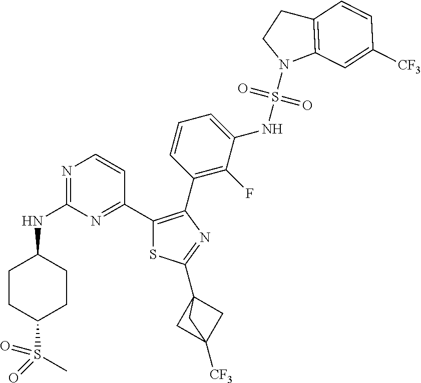 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(C(F)(F)F)cc54)c3F)n2)CC1] | N-(2-fluoro-3- (5-(2-(((1r, 4r)-4- (methylsulfonyl) cyclohexyl) amino) pyrimidin-4- yl)-2-(3- (trifluoromethyl) bicyclo- [1.1.1]-pentan- 1-yl)thiazol-4- yl)phenyl)-6- (trifluoromethyl) indoline-1- sulfonamide | 830.9 |
| 56 | 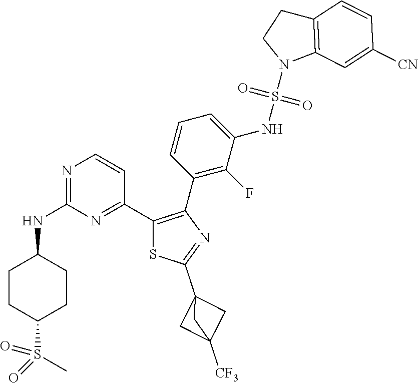 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(C#N)cc54)c3F)n2)CC1] | 6-cyano-N-(2- fluoro-3-(5-(2- (((1r,4r)-4- (methylsulfonyl) cyclohexyl) amino)- pyrimidin-4- yl)-2-(3- (trifluoromethyl)- bicyclo[1.1.1]- pentan- 1-yl)thiazol-4- yl)phenyl)indoline- 1-sulfonamide | 788.2 |
| 57 | 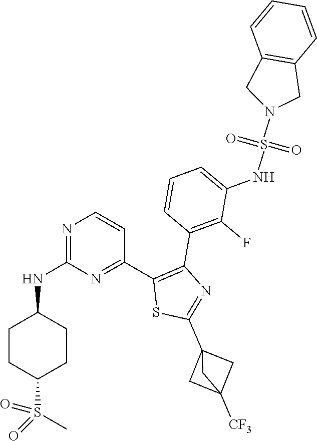 [SMILES: CS(=O)(=O)[C@H]1CC[C@H](Nc2nccc(-c3sc(C45CC(C(F)(F)F)(C4)C5)nc3-c3cccc(NS(=O)(=O)N4Cc5ccccc5C4)c3F)n2)CC1] | N-(2-fluoro-3-(5- (2-(((1r,4r)-4- (methylsulfonyl) cyclohexyl)amino)- pyrimidin-4- yl)-2-(3- (trifluoromethyl)- bicyclo[1.1.1]- pentan- 1-yl)thiazol-4- yl)phenyl) isoindoline-2- sulfonamide | 763.4 |
| 58 | 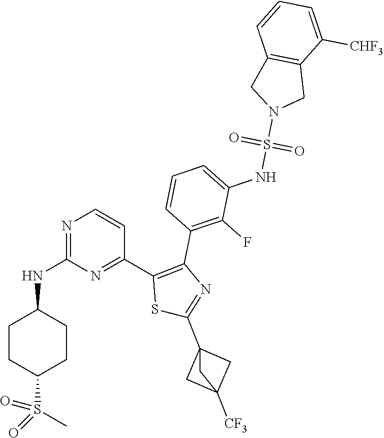 | 4- (difluoromethyl)- N-(2-fluoro- 3-(5-(2- (((1r,4r)-4- (methylsulfonyl) cyclohexyl)- amino)pyrimidin- 4-yl)-2-(3- (trifluoromethyl)- bicyclo[1.1.1] pentan-1- yl)thiazol-4- yl)phenyl)- isoindoline-2- sulfonamide | 813.2 |
| 59 | 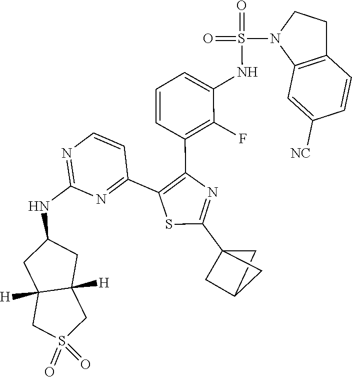 [SMILES: N#Cc1ccc2c(c1)N(S(=O)(=O)Nc1cccc(-c3nc(C45CC(C4)C5)sc3-c3ccnc(N[C@H]4C[C@@H]5CS(=O)(=O)C[C@@H]5C4)n3)c1F)CC2] | N-(3-(2-(bicyclo [1.1.1]pentan- 1-yl)-5-(2- (((3aR,5s,6aS)- 2,2- dioxidohexahydro- 1H-cyclopenta[c] thiophen-5- yl)amino)- pyrimidin-4- yl)thiazol-4- yl)-2- fluorophenyl)-6- cyanoindoline- 1-sulfonamide | 718.2 |
| 60 | 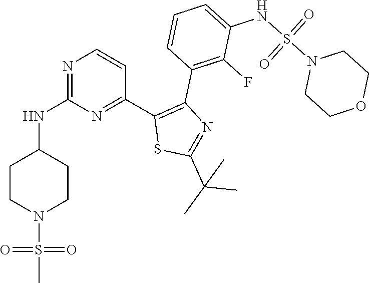 [SMILES: CC(C)(C)c1nc(-c2cccc(NS(=O)(=O)N3CCOCC3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl) morpholine-4- sulfonamide | 654.3 |
| 61 | 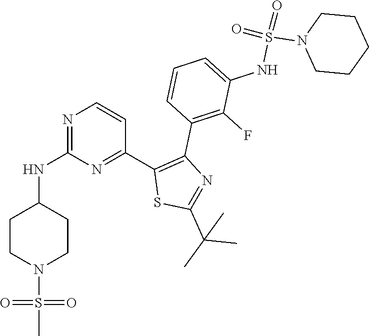 [SMILES: CC(C)(C)c1nc(-c2cccc(NS(=O)(=O)N3CCCCC3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)piperidine- 1-sulfonamide | 652.3 |
| 62 | 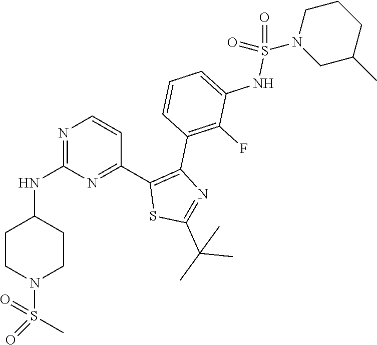 [SMILES: CC1CCCN(S(=O)(=O)Nc2cccc(-c3nc(C(C)(C)C)sc3-c3ccnc(NC4CCN(S(C)(=O)=O)CC4)n3)c2F)C1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-3- methylpiperidine- 1-sulfonamide | 666.4 |
| 63 | 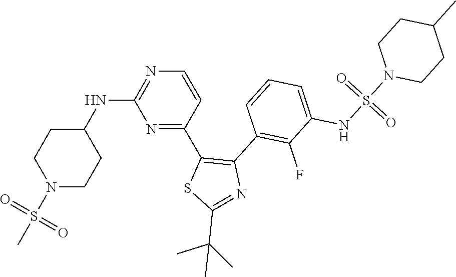 [SMILES: CC1CCN(S(=O)(=O)Nc2cccc(-c3nc(C(C)(C)C)sc3-c3ccnc(NC4CCN(S(C)(=O)=O)CC4)n3)c2F)CC1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- methylpiperidine- 1-sulfonamide | 666.3 |
| 64 | 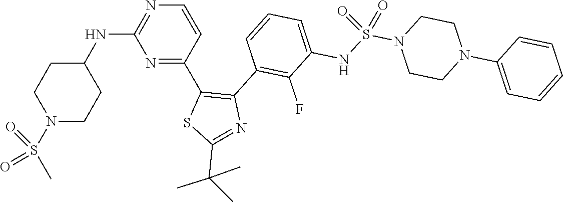 [SMILES: CC(C)(C)c1nc(-c2cccc(NS(=O)(=O)N3CCN(c4ccccc4)CC3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- phenylpiperazine- 1-sulfonamide | 729.2 |
| 65 | 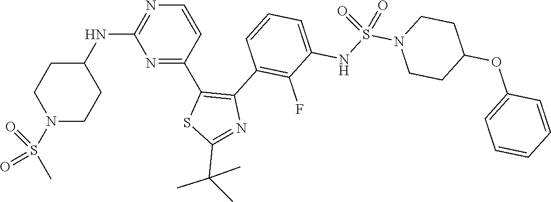 [SMILES: CC(C)(C)c1nc(-c2cccc(NS(=O)(=O)N3CCC(Oc4ccccc4)CC3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- phenoxypiperidine- 1-sulfonamide | 744.3 |
| 66 | 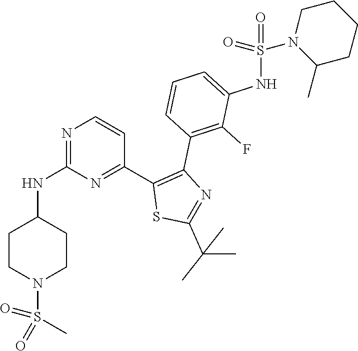 [SMILES: CC1CCCCN1S(=O)(=O)Nc1cccc(-c2nc(C(C)(C)C)sc2-c2ccnc(NC3CCN(S(C)(=O)=O)CC3)n2)c1F] | N-(3-(2-(tert-butyl)- 5-(2-((1- (methylsulfonyl)- piperidin-4- yl)amino)- pyrimidin-4- yl)thiazol-4- yl)-2-fluoro- phenyl)-2- methylpiperidine- 1-sulfonamide | 666.4 |
| 67 | 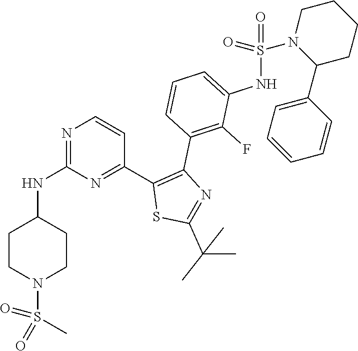 [SMILES: CC(C)(C)c1nc(-c2cccc(NS(=O)(=O)N3CCCCC3c3ccccc3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2- phenylpiperidine- 1-sulfonamide | 728.3 |
| 68 | 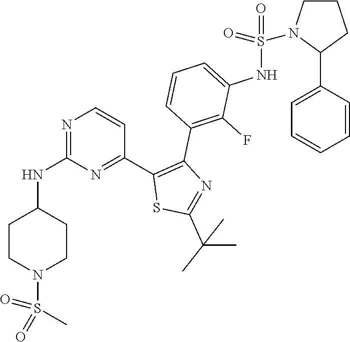 [SMILES: CC(C)(C)c1nc(-c2cccc(NS(=O)(=O)N3CCCC3c3ccccc3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2- phenylpyrrolidine- 1-sulfonamide | 714.3 |
| 69 | 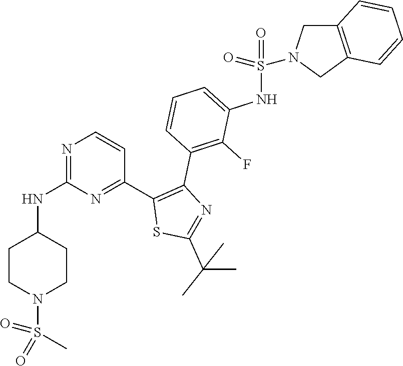 [SMILES: CC(C)(C)c1nc(-c2cccc(NS(=O)(=O)N3Cc4ccccc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl) isoindoline- 2-sulfonamide | 686.3 |
| 70 | 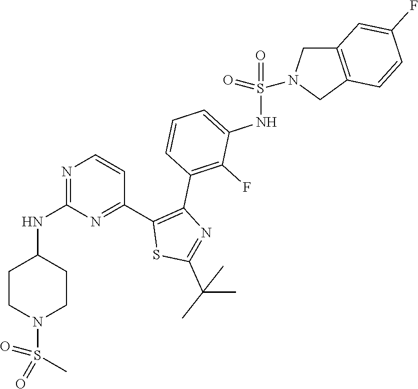 [SMILES: CC(C)(C)c1nc(-c2cccc(NS(=O)(=O)N3Cc4ccc(F)cc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- fluoroisoindoline- 2-sulfonamide | 704.3 |
| 71 | 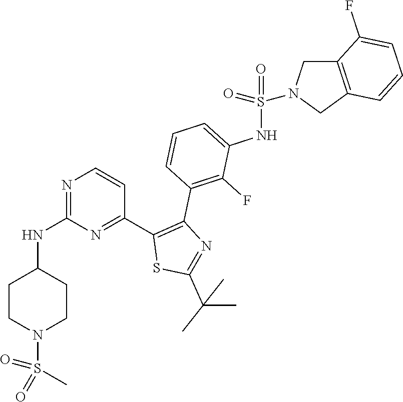 [SMILES: CC(C)(C)c1nc(-c2cccc(NS(=O)(=O)N3Cc4cccc(F)c4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- fluoroisoindoline- 2-sulfonamide | 704.3 |
| 72 | 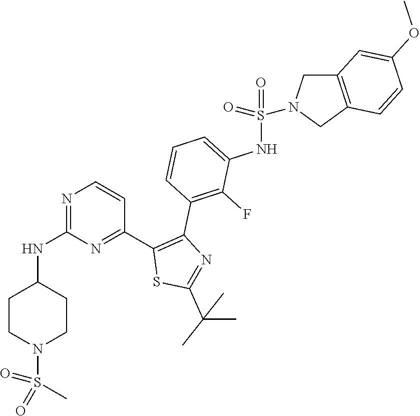 [SMILES: COc1ccc2c(c1)CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- methoxyisoindoline- 2-sulfonamide | 716.3 |

| | | | |
|---|---|---|---|
| 73 | 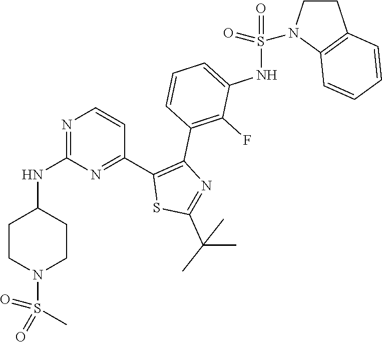 [SMILES: CC(C)(C)c1nc(-c2cccc(NS(=O)(=O)N3CCc4ccccc43)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)indoline- 1-sulfonamide | 686.3 |

| | | | |
|---|---|---|---|
| 74 | 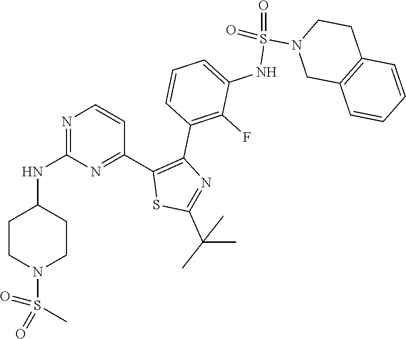 [SMILES: CC(C)(C)c1nc(-c2cccc(NS(=O)(=O)N3CCc4ccccc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-3,4- dihydro- isoquinoline- 2(1H)- sulfonamide | 700.4 |

| | | | |
|---|---|---|---|
| 75 | 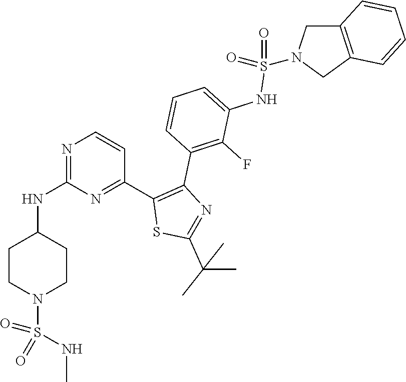 [SMILES: CNS(=O)(=O)N1CCC(Nc2nccc(-c3sc(C(C)(C)C)nc3-c3cccc(NS(=O)(=O)N4Cc5ccccc5C4)c3F)n2)CC1] | N-(3-(2- (tert-butyl)- 5-(2-((1-(N- methylsulfamoyl) piperidin-4- yl)amino)- pyrimidin-4- yl)thiazol-4- yl)-2-fluoro- phenyl)- isoindoline- 2-sulfonamide | 701.3 |

| | | | |
|---|---|---|---|
| 76 | 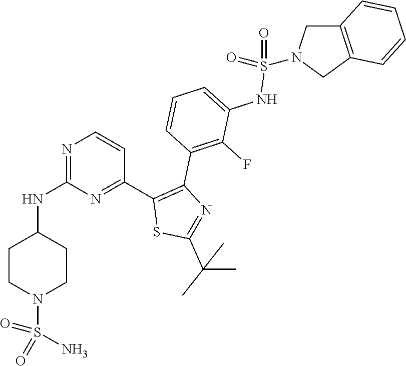 [SMILES: CC(C)(C)c1nc(-c2cccc(NS(=O)(=O)N3Cc4ccccc4C3)c2F)c(-c2ccnc(NC3CCN(S(N)(=O)=O)CC3)n2)s1] | N-(3-(2- (tert-butyl)- 5-(2-((1- sulfamoyl- piperidin-4- yl)amino) pyrimidin-4- yl)thiazol-4- yl)-2- fluorophenyl)- isoindoline-2- sulfonamide | 687.2 |
| 77 | 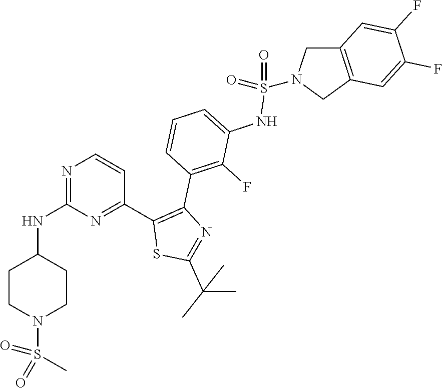 [SMILES: CC(C)(C)c1nc(-c2cccc(NS(=O)(=O)N3Cc4cc(F)c(F)cc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5,6- difluoroisoindoline- 2-sulfonamide | 722.3 |
| 78 | 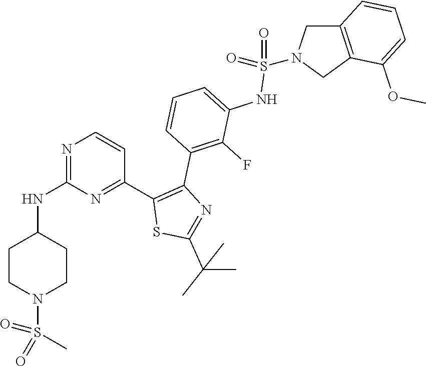 [SMILES: COc1cccc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- methoxyisoindoline- 2-sulfonamide | 716.3 |
| 79 | 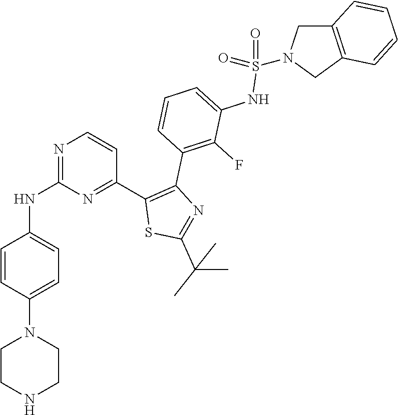 [SMILES: CC(C)(C)c1nc(-c2cccc(NS(=O)(=O)N3Cc4ccccc4C3)c2F)c(-c2ccnc(Nc3ccc(N4CCNCC4)cc3)n2)s1] | N-(3-(2- (tert-butyl)- 5-(2-((4- (piperazin- 1-yl)-phenyl) amino) pyrimidin-4- yl)thiazol-4- yl)-2- fluorophenyl)- isoindoline- 2-sulfonamide | 685.3 |
| 80 | 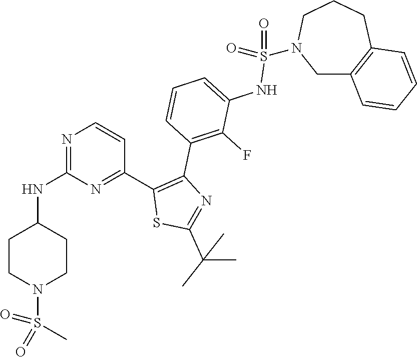 [SMILES: CC(C)(C)c1nc(-c2cccc(NS(=O)(=O)N3CCCc4ccccc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2- fluorophenyl)- 1,3,4,5- tetrahydro-2H- benzo[c]azepine- 2-sulfonamide | 714.4 |
| 81 | 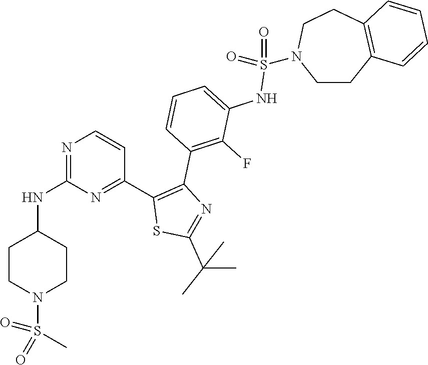 [SMILES: CC(C)(C)c1nc(-c2cccc(NS(=O)(=O)N3CCc4ccccc4CC3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-1,2,4,5- tetrahydro-3H- benzo[d]azepine- 3-sulfonamide | 714.3 |
| 82 | 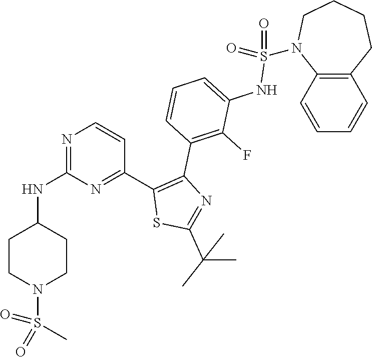 [SMILES: CC(C)(C)c1nc(-c2cccc(NS(=O)(=O)N3CCCCc4ccccc43)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)- 2,3,4,5- tetrahydro-1H- benzo[b]azepine- 1-sulfonamide | 714.3 |
| 83 | 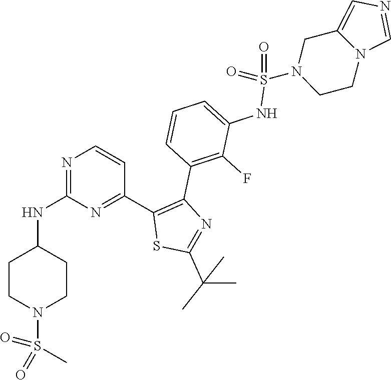 [SMILES: CC(C)(C)c1nc(-c2cccc(NS(=O)(=O)N3CCn4cncc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5,6- dihydroimidazo [1,5-a]- pyrazine-7 (8H)- sulfonamide | 690.3 |
| 84 | 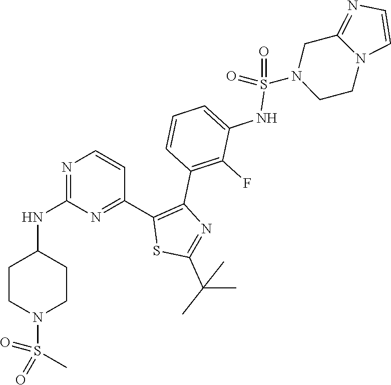 [SMILES: CC(C)(C)c1nc(-c2cccc(NS(=O)(=O)N3CCn4ccnc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5,6- dihydroimidazo [1,2-a]- pyrazine-7(8H)- sulfonamide | 690.3 |
| 85 | 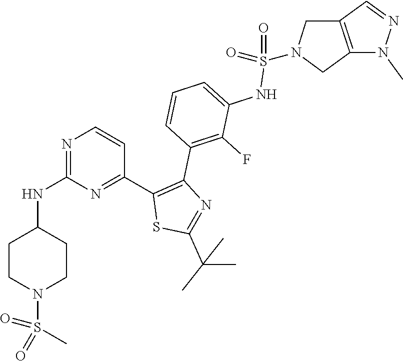 [SMILES: Cn1ncc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-1- methyl-4,6- dihydro- pyrrolo[3,4- c]pyrazole-5(1H)- sulfonamide | 690.3 |
| 86 | 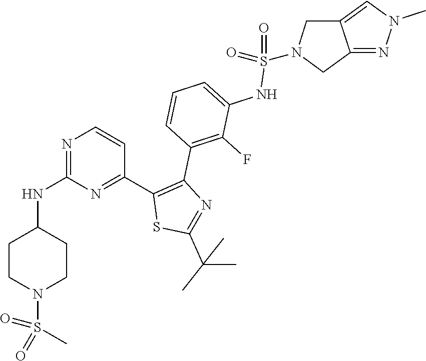 [SMILES: Cn1cc2c(n1)CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2- methyl-2,6- dihydro- pyrrolo[3,4- c]pyrazole-5(4H)- sulfonamide | 690.3 |
| 87 | 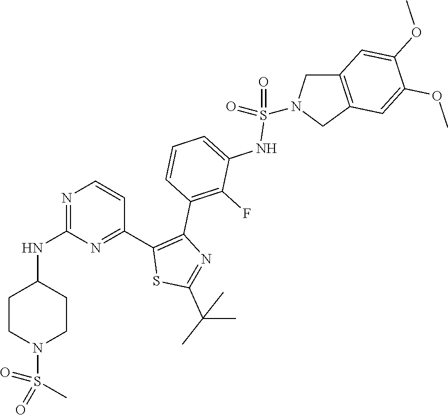 [SMILES: COc1cc2c(cc1OC)CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2- fluorophenyl)- 5,6- dimethoxy- isoindoline- 2-sulfonamide | 746.3 |
| 88 | 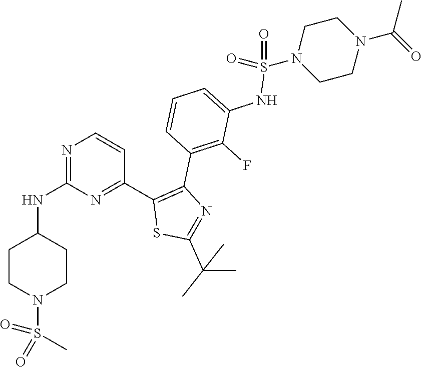 [SMILES: CC(=O)N1CCN(S(=O)(=O)Nc2cccc(-c3nc(C(C)(C)C)sc3-c3ccnc(NC4CCN(S(C)(=O)=O)CC4)n3)c2F)CC1] | 4-acetyl-N- (3-(2-(tert- butyl)- 5-(2-((1- (methylsulfonyl) piperidin-4- yl)amino)- pyrimidin-4- yl)thiazol-4- yl)-2-fluoro- phenyl)- piperazine- 1-sulfonamide | 695.3 |
| 89 | 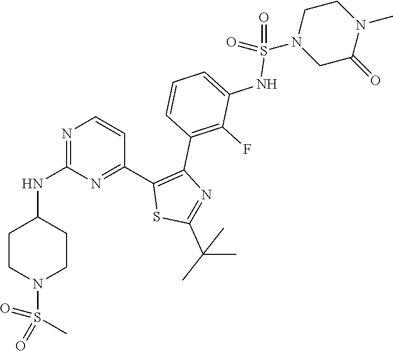 [SMILES: CN1CCN(S(=O)(=O)Nc2cccc(-c3nc(C(C)(C)C)sc3-c3ccnc(NC4CCN(S(C)(=O)=O)CC4)n3)c2F)CC1=O] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- methyl-3- oxopiperazine- 1- sulfonamide | 681.3 |
| 90 | 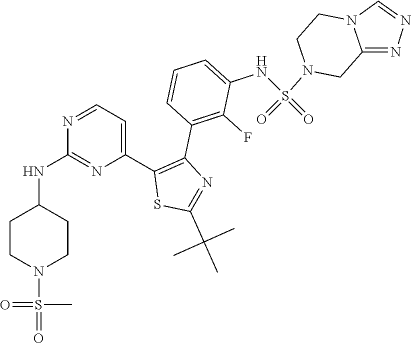 [SMILES: CC(C)(C)c1nc(-c2cccc(NS(=O)(=O)N3CCn4cnnc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)~2~fluoro- phenyl)-5,6- dihydro-[1,2,4] triazolo- [4,3-a]pyrazine- 7(8H)- sulfonamide | 691.3 |
| 91 | 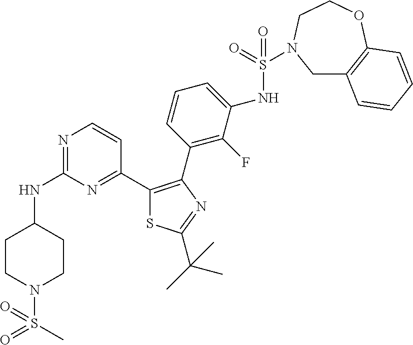 [SMILES: CC(C)(C)c1nc(-c2cccc(NS(=O)(=O)N3CCOc4ccccc4C3)c2F)c(-c2ccnc(NC3CCN(S(C)(=O)=O)CC3)n2)s1] | N-(3-(2-(tert- butyl)- 5-(2-((1- (methylsulfonyl)- piperidin-4- yl)amino)- pyrimidin-4- yl)thiazol-4- yl)-2- fluorophenyl)- 2,3-dihydrobenzo [f][1,4]- oxazepine-4 (5H)-sulfonamide | 716.3 |
| 92 | 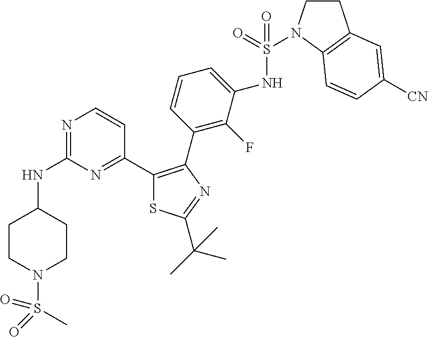 [SMILES: CC(C)(C)c1nc(-c2cccc(NS(=O)(=O)N3CCc4cc(C#N)ccc43)c2F)c(-c2ccnc(NC3CCN(S(C)(=O)=O)CC3)n2)s1] | N-(3-(2-(tert- butyl)- 5-(2-((1- (methylsulfonyl) piperidin-4- yl)amino)- pyrimidin-4- yl)thiazol-4- yl)-2- fluorophenyl)- 5-cyanoindoline- 1-sulfonamide | |
| 93 | 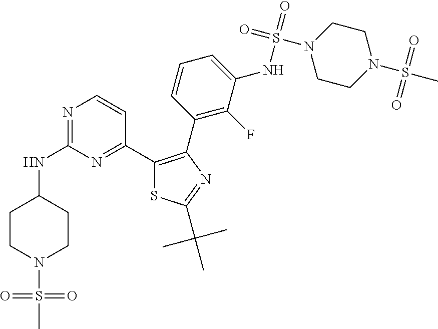 [SMILES: CC(C)(C)c1nc(-c2cccc(NS(=O)(=O)N3CCN(S(C)(=O)=O)CC3)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)-4- (methylsulfonyl)- piperazine- 1-sulfonamide | 731.2 |
| 94 | 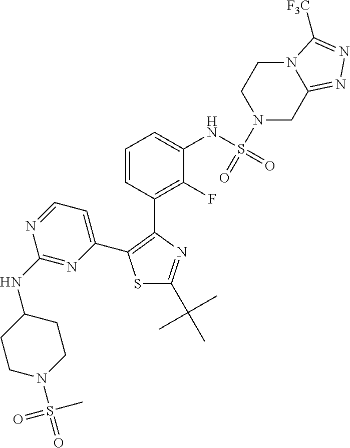 [SMILES: CC(C)(C)c1nc(-c2cccc(NS(=O)(=O)N3CCn4c(nnc4C(F)(F)F)C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-3- (trifluoromethyl)- 5,6-dihydro- [1,2,4]triazolo [4,3-a]pyrazine- 7(8H)- sulfonamide | 759.3 |
| 95 | 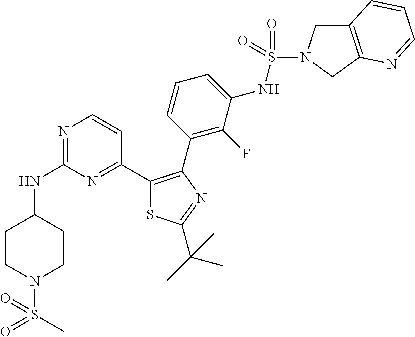 [SMILES: CC(C)(C)c1nc(-c2cccc(NS(=O)(=O)N3Cc4cccnc4C3)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2-fluoro- phenyl)-5,7- dihydro-6H- pyrrolo- [3,4-b]pyridine- 6-sulfonamide | 687.2 |
| 96 | 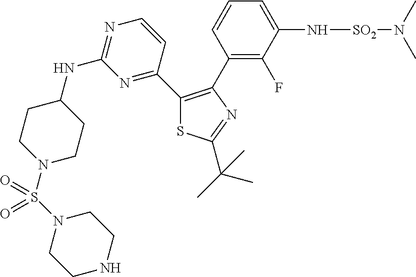 | N-(3-(2- (tert-butyl)- 5-(2-((1- (piperazin- 1-ylsulfonyl) piperidin-4- yl)amino)- pyrimidin-4- yl)thiazol-4- yl)-2- fluorophenyl) dimethylamine- sulfonamide | 682.2 |
| 97 | 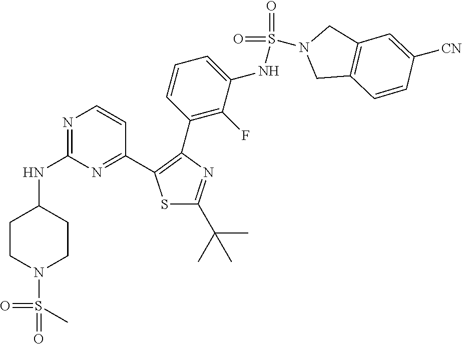 [SMILES: CC(C)(C)c1nc(-c2cccc(NS(=O)(=O)N3Cc4ccc(C#N)cc4C3)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)- 5-cyanoisoindoline- 2- sulfonamide | 711.1 |
| 98 | 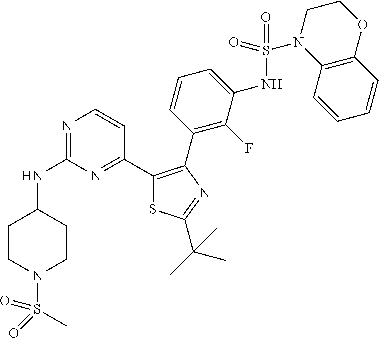 [SMILES: CC(C)(C)c1nc(-c2cccc(NS(=O)(=O)N3CCOc4ccccc43)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2-fluoro- phenyl)-2,3- dihydro-4H- benzo[b][1,4]- oxazine-4- sulfonamide | 702.1 |
| 99 | 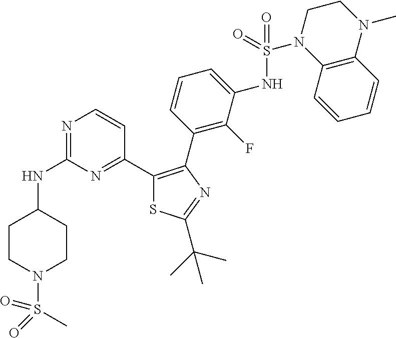 [SMILES: CN1CCN(S(=O)(=O)Nc2cccc(-c3nc(C(C)(C)C)sc3-c3ccnc(NC4CCN(S(C)(=O)=O)CC4)n3)c2F)c2ccccc21] | N-(3-(2- (tert-butyl)- 5-(2-((1- (methylsulfonyl) piperidin-4- yl)amino)- pyrimidin-4- yl)thiazol-4- yl)-2-fluoro- phenyl)-4- methyl-3,4- dihydro- quinoxaline- 1(2H)- sulfonamide | 715.2 |
| 100 | 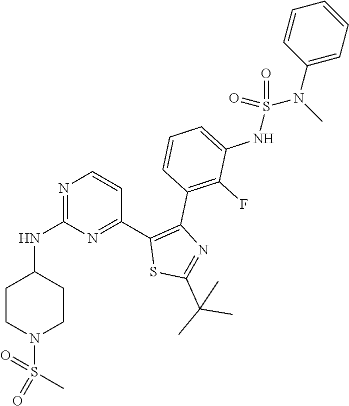 [SMILES: CN(c1ccccc1)S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl) piperidin-4- yl)amino)- pyrimidin-4- yl)thiazol-4- yl)-2- fluorophenyl)(N- methyl-N-phenyl)- aminosulfonamide | 674.2 |
| 101 | 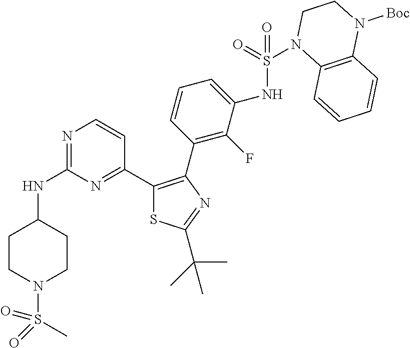 [SMILES: CC(C)(C)OC(=O)N1CCN(S(=O)(=O)Nc2cccc(-c3nc(C(C)(C)C)sc3-c3ccnc(NC4CCN(S(C)(=O)=O)CC4)n3)c2F)c2ccccc21] | tert-butyl 4-(N- (3-(2-(tert-butyl)- 5-(2-((1- (methylsulfonyl) piperidin-4- yl)amino)- pyrimidin-4- yl)thiazol-4- yl)-2- fluorophenyl)- sulfamoyl)-3,4- dihydroquinoxaline- 1(2H)-carboxylate | 801.3 |
| 102 | 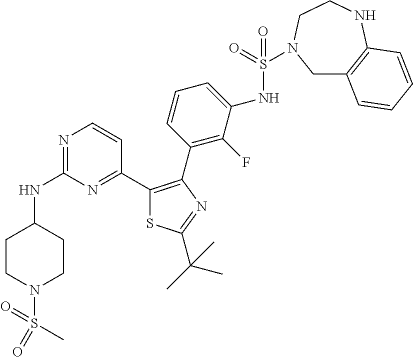 [SMILES: CC(C)(C)c1nc(-c2cccc(NS(=O)(=O)N3CCNc4ccccc4C3)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)- 1,2,3,5- tetrahydro-4H- benzo[e] [1,4]diazepine- 4-sulfonamide | 715.2 |
| 103 | 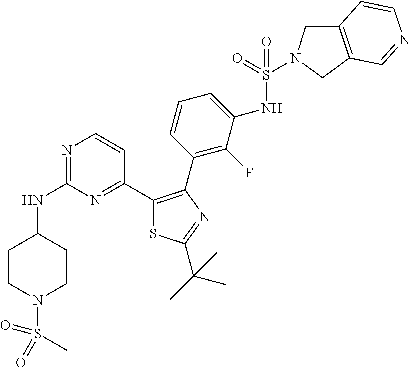 [SMILES: CC(C)(C)c1nc(-c2cccc(NS(=O)(=O)N3Cc4ccncc4C3)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2 fluorophenyl)- 1,3-dihydro-2H- pyrrolo[3,4-c] pyridine-2- sulfonamide | 687.2 |
| 104 | 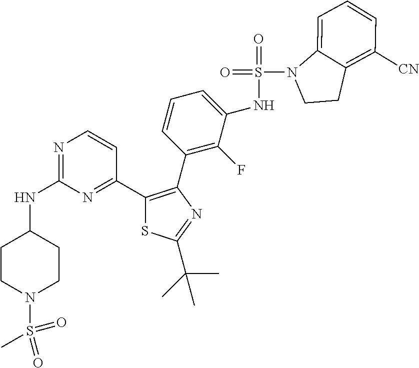 [SMILES: CC(C)(C)c1nc(-c2cccc(NS(=O)(=O)N3CCc4c(C#N)cccc43)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)-4- cyanoindoline- 1- sulfonamide | 711.2 |
| 105 | 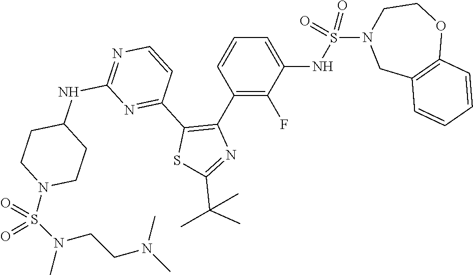 [SMILES: CN(C)CCN(C)S(=O)(=O)N1CCC(Nc2nccc(-c3sc(C(C)(C)C)nc3-c3cccc(NS(=O)(=O)N4CCOc5ccccc5C4)c3F)n2)CC1] | N-(3-(2- (tert-butyl)- 5-(2-((1-(N- (2-(dimethyl- amino) ethyl)-N- methyl- sulfamoyl) piperidin-4- yl)amino)- pyrimidin-4- yl)thiazol-4- yl)-2-fluoro- phenyl)-2,3- dihydrobenzo [f][1,4]- oxazepine-4 | 802.3 |
| | | (5H)-sulfonamide | |
| 106 | 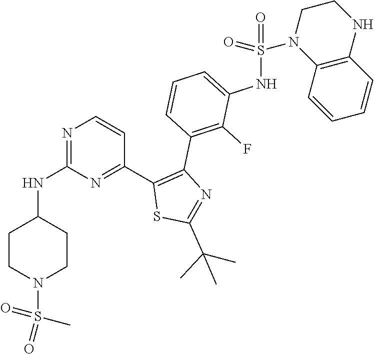 [SMILES: CC(C)(C)c1nc(-c2cccc(NS(=O)(=O)N3CCNc4ccccc43)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2- fluorophenyl)- 3,4- dihydroquinoxaline- 1(2H)- sulfonamide | 701.2 |
| 107 | 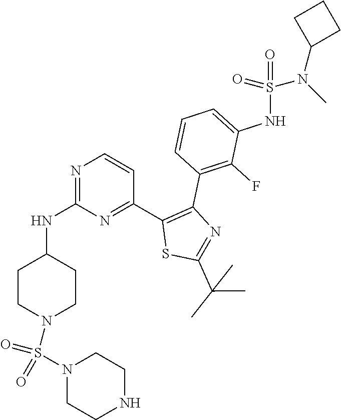 [SMILES: CN(C1CCC1)S(=O)(=O)Nc1cccc(-c2nc(C(C)(C)C)sc2-c2ccnc(NC3CCN(S(=O)(=O)N4CCNCC4)CC3)n2)c1F] | N-(3-(2- (tert-butyl)- 5-(2-((1- (piperazin- 1-yl-sulfonyl) piperidin-4- yl)amino)- pyrimidin-4- yl)thiazol-4- yl)-2-fluoro- phenyl)(N- methyl-N- cyclo- butylamine)- sulfonamide | 722.2 |
| 108 | 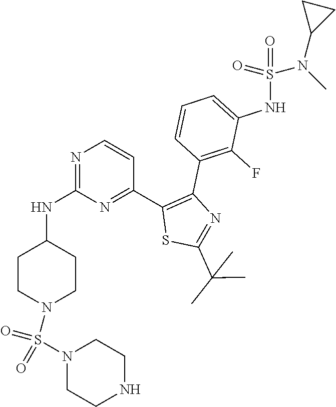 [SMILES: CN(C1CC1)S(=O)(=O)Nc1cccc(-c2nc(C(C)(C)C)sc2-c2ccnc(NC3CCN(S(=O)(=O)N4CCNCC4)CC3)n2)c1F] | N-(3-(2-(tert- butyl)- 5-(2-((1- (piperazin- 1-yl-sulfonyl) piperidin-4- yl)amino)- pyrimidin-4- yl)thiazol-4- yl)-2-fluoro- phenyl)(N- methyl-N- cyclopro- pylamine)- sulfonamide | 708.2 |
| 109 | 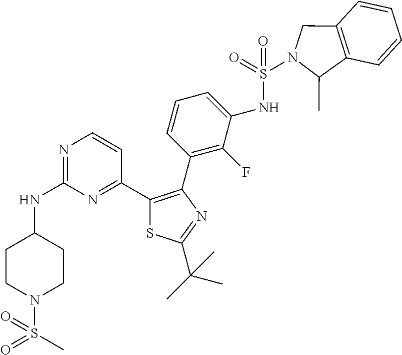 [SMILES: CC1c2ccccc2CN1S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyrimidin-4- yl)thiazol-4- yl)-2-fluoro- phenyl)-1- methylisoindoline- 2-sulfonamide | 700.2 |
| 110 | 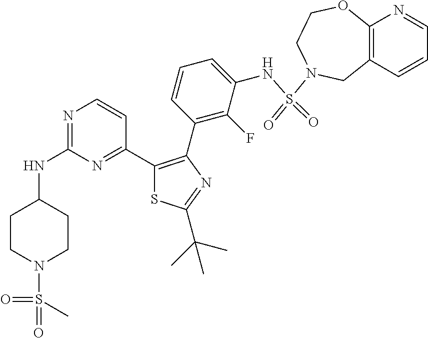 [SMILES: CC(C)(C)c1nc(-c2cccc(NS(=O)(=O)N3CCOc4ncccc4C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2,3- dihydropyrido [3,2-f][1,4]- oxazepine-4 (5H)-sulfonamide | 717.2 |
| 111 | 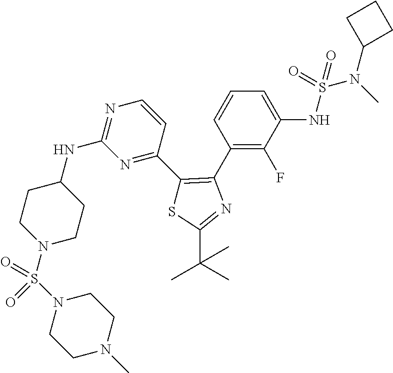 [SMILES: CN1CCN(S(=O)(=O)N2CCC(Nc3nccc(-c4sc(C(C)(C)C)nc4-c4cccc(NS(=O)(=O)N(C)C5CCC5)c4F)n3)CC2)CC1] | N-(3-(2- (tert-butyl)- 5-(2-((1-((4- methylpiperazin- 1-yl)sulfonyl) piperidin-4- yl)amino) pyrimidin-4- yl)thiazol-4- yl)-2- fluorophenyl)- (N-methyl-N- cyclobutylamine)- sulfonamide | 736.4 |
| 112 | 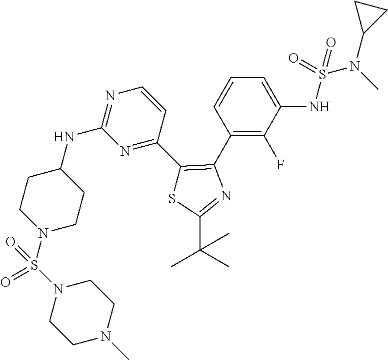 [SMILES: CN1CCN(S(=O)(=O)N2CCC(Nc3nccc(-c4sc(C(C)(C)C)nc4-c4cccc(NS(=O)(=O)N(C)C5CC5)c4F)n3)CC2)CC1] | N-(3-(2- (tert-butyl)- 5-(2-((1-((4- methylpiperazin- 1-yl)sulfonyl) piperidin-4- yl)amino) pyrimidin-4- yl)thiazol-4- yl)-2- fluorophenyl)- (N-methyl-N- cyclopropylamine)- sulfonamide | 722.3 |
| 113 | 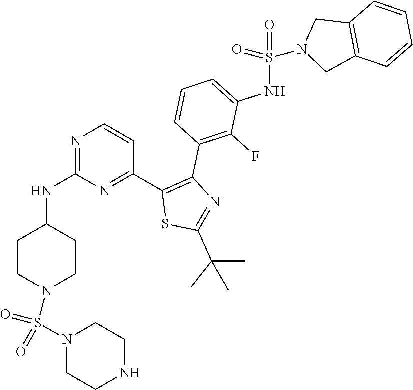 [SMILES: CC(C)(C)c1nc(-c2cccc(NS(=O)(=O)N3Cc4ccccc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)isoindoline- 2-sulfonamide | 756.2 |
| 114 | 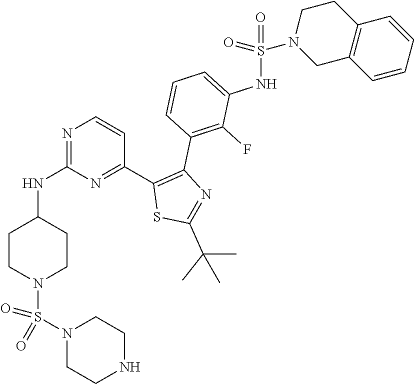 [SMILES: CC(C)(C)c1nc(-c2cccc(NS(=O)(=O)N3CCc4ccccc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-3,4- dihydro- isoquinoline- 2(1H)- sulfonamide | 770.4 |
| 115 | 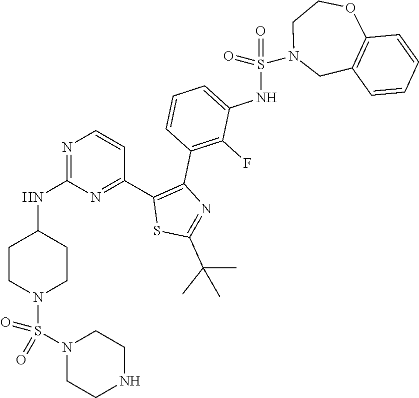 [SMILES: CC(C)(C)c1nc(-c2cccc(NS(=O)(=O)N3CCOc4ccccc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2- fluorophenyl)- 2,3- dihydrobenzo [f][1,4]- oxazepine- 4(5H)- sulfonamide | 786.2 |
| 116 | 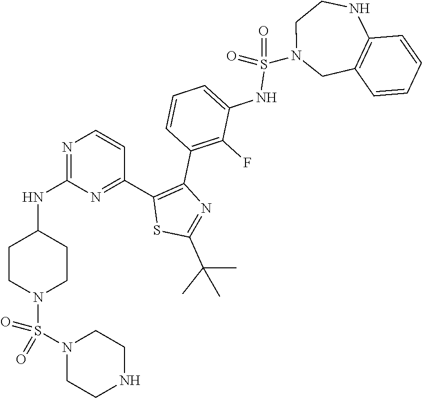 [SMILES: CC(C)(C)c1nc(-c2cccc(NS(=O)(=O)N3CCNc4ccccc4C3)c2F)c(-c2ccnc(NC3CCN(S(=O)(=O)N4CCNCC4)CC3)n2)s1] | N-(3-(2- (tert-butyl)- S-(2-((1- (piperazin- 1-ylsulfonyl) piperidin-4- yl)amino)- pyrimidin-4- yl)thiazol-4- yl)-2- fluorophenyl)- 1,2,3,5- tetrahydro-4H- benzo[e][1,4] diazepine- 4-sulfonamide | 785.2 |
| 117 | 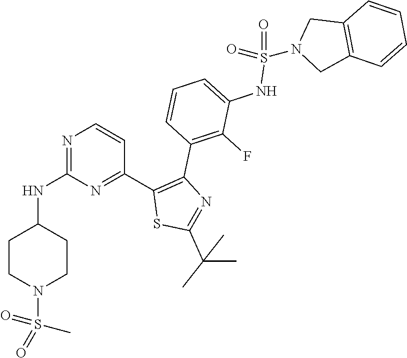 [SMILES: CC(C)(C)c1nc(-c2cccc(NS(=O)(=O)N3Cc4ccccc4C3)c2F)c(-c2ccnc(NC3CCN(S(C)(=O)=O)CC3)n2)s1] | N-(3-(2-(tert- butyl)- 5-(2-((4- (methylsulfonyl)- cyclohexyl) amino)- pyrimidin-4- yl)thiazol-4- yl)-2-fluoro- phenyl) isoindoline- 2-sulfonamide | 685.1 |
| 118 | 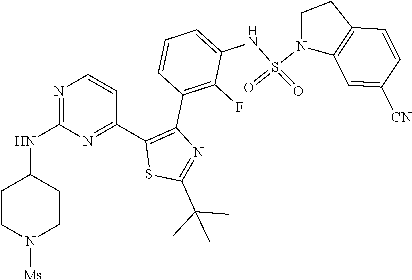 [SMILES: CC(C)(C)c1nc(-c2cccc(NS(=O)(=O)N3CCc4ccc(C#N)cc43)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2- fluorophenyl)- 6-cyanoindoline- 1-sulfonamide | 711.2 |
| 119 | 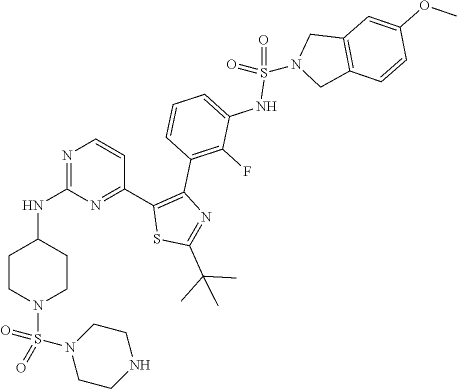 [SMILES: COc1ccc2c(c1)CN(S(=O)(=O)Nc1cccc(-c3nc(C(C)(C)C)sc3-c3ccnc(NC4CCN(S(=O)(=O)N5CCNCC5)CC4)n3)c1F)C2] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-5- methoxy- isoindoline-2- sulfonamide | 786.4 |
| 120 | 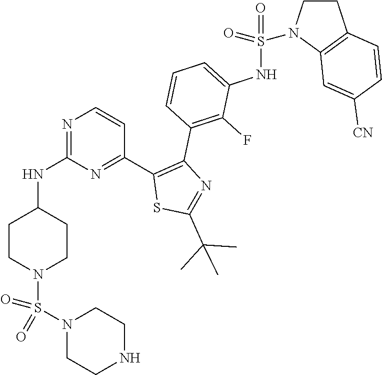 [SMILES: CC(C)(C)c1nc(-c2cccc(NS(=O)(=O)N3CCc4ccc(C#N)cc43)c2F)c(-c2ccnc(NC3CCN(S(=O)(=O)N4CCNCC4)CC3)n2)s1] | N-(3-(2-(tert-butyl)- 5-(2-((1-(piperazin- 1-ylsulfonyl) piperidin-4- yl)amino)- pyrimidin-4- yl)thiazol-4- yl)-2- fluorophenyl)-6- cyanoindoline- 1-sulfonamide | 781.2 |
| 121 | 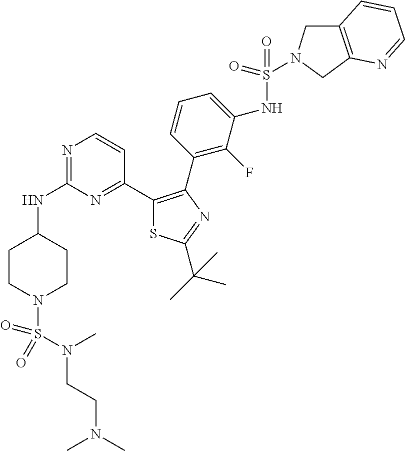 [SMILES: CN(C)CCN(C)S(=O)(=O)N1CCC(Nc2nccc(-c3sc(C(C)(C)C)nc3-c3cccc(NS(=O)(=O)N4Cc5cccnc5C4)c3F)n2)CC1] | N-(3-(2-(tert-butyl)- 5-(2-((1~(N-(2- (dimethylamino) ethyl)-N- methylsulfamoyl)- piperidin-4- yl)amino)- pyrimidin-4- yl)thiazol-4- yl)-2-fluoro- phenyl)-5,7-dihydro- 6H-pyrrolo[3,4-b]- pyridine-6-sulfonamide | 773.4 |
| 122 | 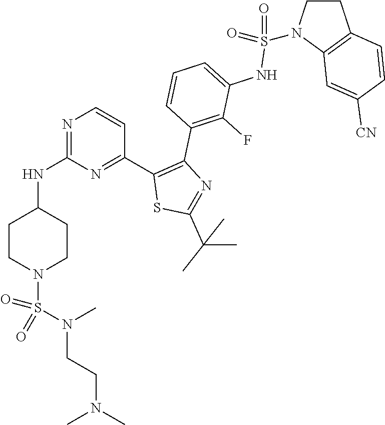 [SMILES: CN(C)CCN(C)S(=O)(=O)N1CCC(Nc2nccc(-c3sc(C(C)(C)C)nc3-c3cccc(NS(=O)(=O)N4CCc5ccc(C#N)cc54)c3F)n2)CC1] | N-(3-(2-(tert-butyl)- 5-(2-((1-(N-(2- (dimethylamino) ethyl)-N- methylsulfamoyl)- piperidin-4- yl)amino)- pyrimidin-4- yl)thiazol-4- yl)-2-fluoro- phenyl)-6- cyanoindoline- 1-sulfonamide | 797.4 |
| 123 | 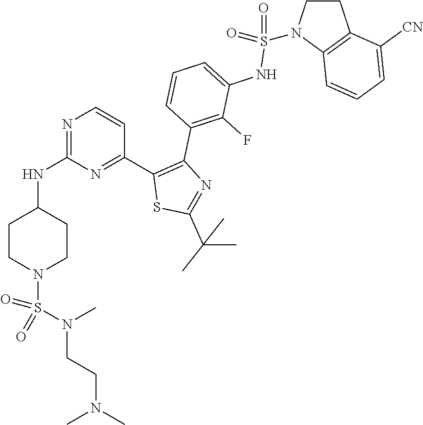 [SMILES: CN(C)CCN(C)S(=O)(=O)N1CCC(Nc2nccc(-c3sc(C(C)(C)C)nc3-c3cccc(NS(=O)(=O)N4CCc5c(C#N)cccc54)c3F)n2)CC1] | N-(3-(2- (tert-butyl)- 5-(2-((1-(N-(2- (dimethylamino) ethyl)-N- methylsulfamoyl)- piperidin-4- yl)amino)- pyrimidin-4- yl)thiazol-4- yl)-2-fluoro- phenyl)-4- cyanoindoline- 1-sulfonamide | 797.4 |
| 124 | 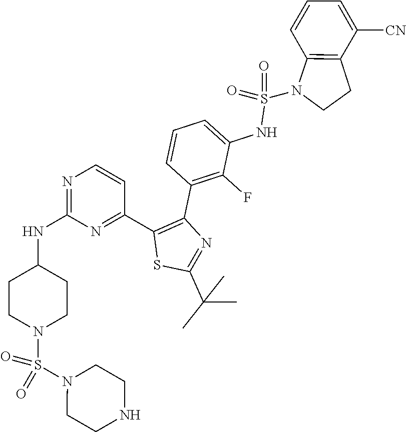 [SMILES: CC(C)(C)c1nc(-c2cccc(NS(=O)(=O)N3CCc4c(C#N)cccc43)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-4- cyanoindoline- 1-sulfonamide | 781.4 |
| 125 | 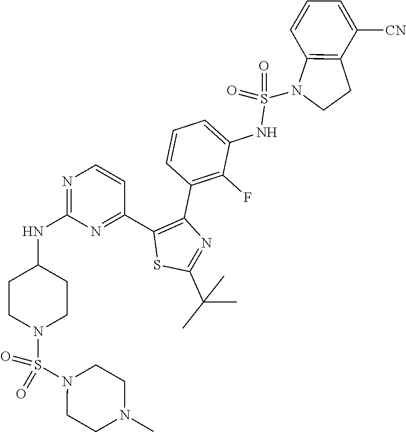 [SMILES: CN1CCN(S(=O)(=O)N2CCC(Nc3nccc(-c4sc(C(C)(C)C)nc4-c4cccc(NS(=O)(=O)N5CCc6c(C#N)cccc65)c4F)n3)CC2)CC1] | N-(3-(2- (tert-butyl)- 5-(2-((1-((4- methyl- piperazin- 1-yl)sulfonyl) piperidin-4- yl)amino)- pyrimidin-4- yl)thiazol-4- yl)-2- fluorophenyl)-4- cyanoindoline- 1-sulfonamide | 795.4 |
| 126 | 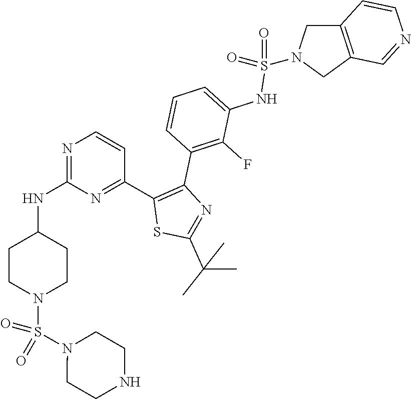 [SMILES: CC(C)(C)c1nc(-c2cccc(NS(=O)(=O)N3Cc4ccncc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2- fluorophenyl)- 1,3-dihydro-2H- pyrrolo[3,4-c] pyridine-2- sulfonamide | 757.3 |
| 127 | 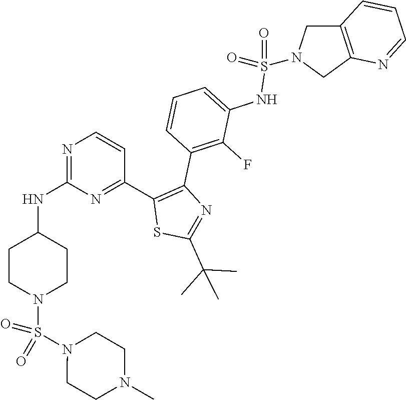 [SMILES: CN1CCN(S(=O)(=O)N2CCC(Nc3nccc(-c4sc(C(C)(C)C)nc4-c4cccc(NS(=O)(=O)N5Cc6cccnc6C5)c4F)n3)CC2)CC1] | N-(3-(2- (tert-butyl)- 5-(2-((1-((4- methyl- piperazin- 1-yl)sulfonyl) piperidin-4- yl)amino)- pyrimidin-4- yl)thiazol-4- yl)-2- fluorophenyl)- 5,7-dihydro-6H- pyrrolo[3,4-b] pyridine-6- sulfonamide | 771.4 |
| 128 | 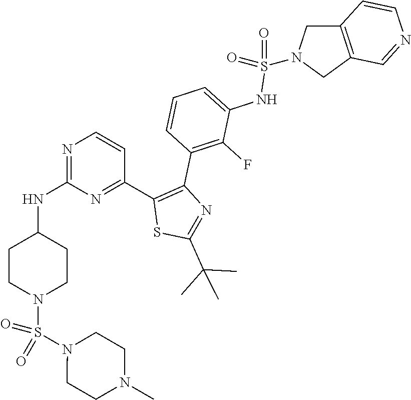 [SMILES: CN1CCN(S(=O)(=O)N2CCC(Nc3nccc(-c4sc(C(C)(C)C)nc4-c4cccc(NS(=O)(=O)N5Cc6ccncc6C5)c4F)n3)CC2)CC1] | N-(3-(2- (tert-butyl)- 5-(2-((1-((4- methylpiperazin- 1-yl)sulfonyl) piperidin- 4.yl)-amino) pyrimidin~4- yl)thiazol-4- yl)- 2-fluorophenyl)- 1,3-dihydro-2H- pyrrolo[3,4-c]- pyridine-2- sulfonamide | 771.4 |
| 129 | 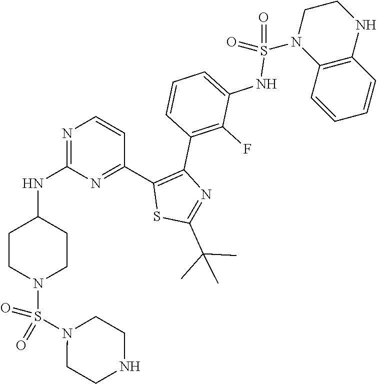 [SMILES: CC(C)(C)c1nc(-c2cccc(NS(=O)(=O)N3CCNc4ccccc43)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-3,4- dihydroquinoxaline- 1(2H)- sulfonamide | 771.4 |
| 130 | 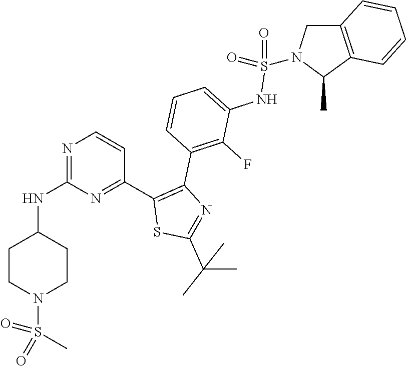 [SMILES: C[C@@H]1c2ccccc2CN1S(=O)(=O)Nc1cccc(-c2nc(C(C)(C)C)sc2-c2ccnc(NC3CCN(S(C)(=O)=O)CC3)n2)c1F] | (R)-N-(3-(2- (tert-butyl)- 5-(2-((1- (methyl- sulfonyl) piperidin-4- yl)amino)- pyrimidin-4- yl)thiazol-4- yl)-2-fluoro- phenyl)-1- methyliso- indoline-2- sulfonamide | 700.2 |
| 131 | 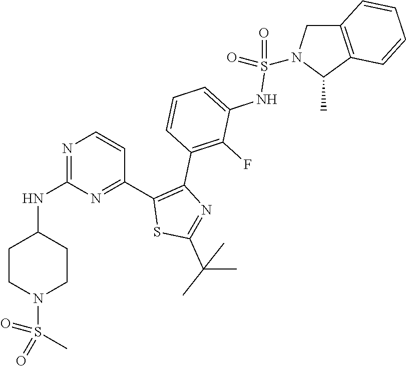 [SMILES: C[C@H]1c2ccccc2CN1S(=O)(=O)Nc1cccc(-c2nc(C(C)(C)C)sc2-c2ccnc(NC3CCN(S(C)(=O)=O)CC3)n2)c1F] | (S)-N-(3- (2-(tert-butyl)- 5-(2-((1- (methyl- sulfonyl) piperidin-4- yl)amino) pyrimidin- 4-yl)thiazol-4- yl)-2-fluoro- phenyl)-1- methyliso- indoline- 2-sulfonamide | 700.2 |
| 132 | 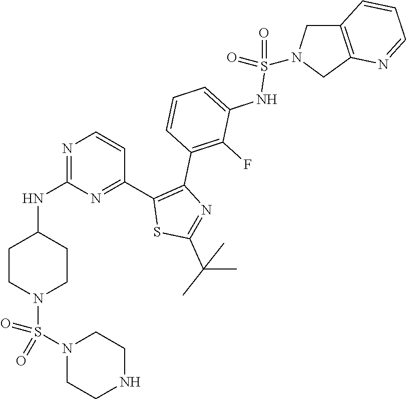 [SMILES: CC(C)(C)c1nc(-c2cccc(NS(=O)(=O)N3Cc4cccnc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-5,7- dihydro-6H- pyrrolo[3,4-b]- pyridine-6- sulfonamide | 757.2 |
| 133 | 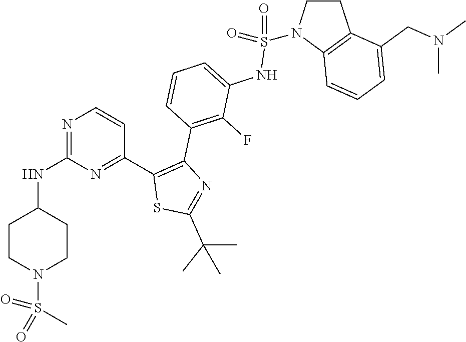 [SMILES: CN(C)Cc1cccc2c1CCN2S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- ((dimethylamino) methyl)- indoline- 1-sulfonamide | 743.4 |
| 134 | 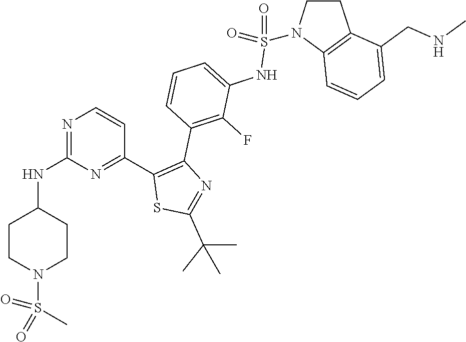 [SMILES: CNCc1cccc2c1CCN2S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyriidin-4- yl)thiazol-4- yl)-2-fluoro- phenyl)-4- ((methylamino) methyl)- indoline- 1-sulfonamide | 729.2 |
| 135 | 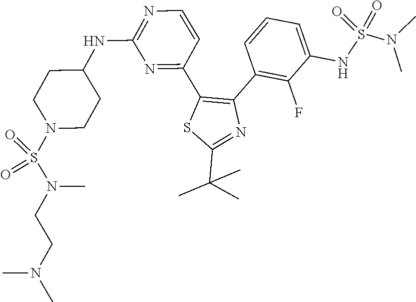 | 4-((4-(2- (tert-butyl)- 4-(3-((N,N- dimethyl- sulfamoyl) amino)-2-fluoro- phenyl)thiazol-5- yl)pyrimidin-2- yl)amino)-N-(2- (dimethyl- amino)ethyl)-N- methylpiperidine- 1-sulfonamide | 698.2 |
| 136 | 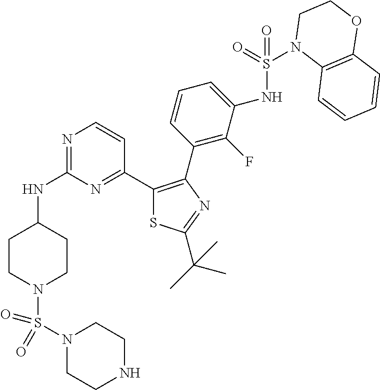 [SMILES: CC(C)(C)c1nc(-c2cccc(NS(=O)(=O)N3CCOc4ccccc43)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2- fluorophenyl)- 2,3-dihydro-4H- benzo[b][1,4] oxazine-4- sulfonamide | 772.2 |
| 137 | 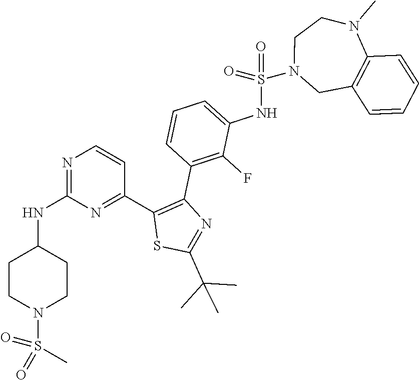 [SMILES: CN1CCN(S(=O)(=O)Nc2cccc(-c3nc(C(C)(C)C)sc3-c3ccnc(NC4CCN(S(C)(=O)=O)CC4)n3)c2F)Cc2ccccc21] | N-(3-(2- (tert-butyl)- 5-(2-((1- (methylsulfonyl)- piperidin-4- yl)amino)- pyrimidin-4- yl)thiazol-4- yl)-2-fluoro- phenyl)-1- methyl-1,2,3,5- tetrahydro-4H- benzo[e][1,4] diazepine- 4-sulfonamide | 729.2 |
| 138 | 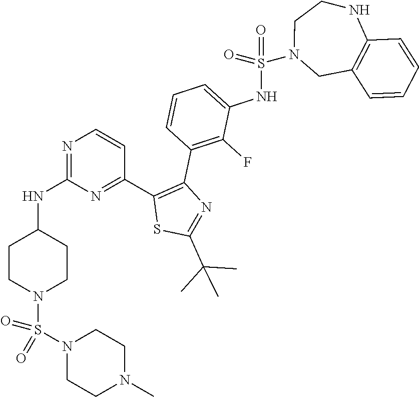 [SMILES: CN1CCN(S(=O)(=O)N2CCC(Nc3nccc(-c4sc(C(C)(C)C)nc4-c4cccc(NS(=O)(=O)N5CCNc6ccccc6C5)c4F)n3)CC2)CC1] | N-(3-(2- (tert-butyl)- 5-(2-((1-((4- methylpiperazin- 1-yl)sulfonyl) piperidin-4- yl)amino) pyrimidin-4- yl)thiazol-4- yl)- 2~fluorophenyl)- 1,2,3,5- tetrahydro-4H- benzo[e][1,4] diazepine- 4-sulfonamide | 799.2 |
| 139 | 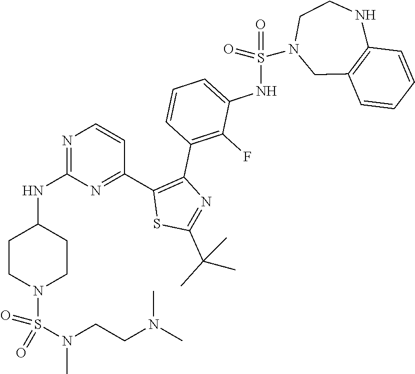 [SMILES: CN(C)CCN(C)S(=O)(=O)N1CCC(Nc2nccc(-c3sc(C(C)(C)C)nc3-c3cccc(NS(=O)(=O)N4CCNc5ccccc5C4)c3F)n2)CC1] | N-(3-(2- (tert-butyl)- 5-(2-((1-(N-(2- (dimethyl- amino)ethyl)- N-methyl- sulfamoyl) piperidin-4- yl)amino)- pyrimidin-4- yl)thiazol-4- yl)-2-fluoro- phenyl)-1,2,3,5- tetrahydro-4H- benzo[e][1,4] diazepine- 4-sulfonamide | 801.3 |
| 140 | 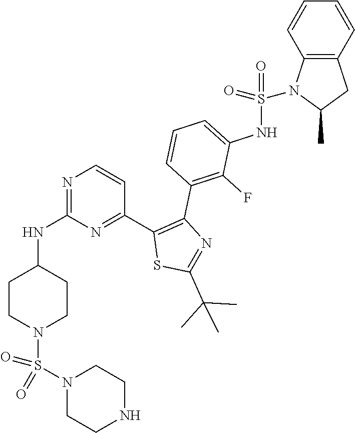 [SMILES: C[C@@H]1Cc2ccccc2N1S(=O)(=O)Nc1cccc(-c2nc(C(C)(C)C)sc2-c2ccnc(NC3CCN(S(=O)(=O)N4CCNCC4)CC3)n2)c1F] | (R)-N-(3-(2- (tert-butyl)- 5-(2-((1- (piperazin- 1-ylsulfonyl) piperidin-4- yl)amino) pyrimidin- 4-yl)thiazol-4- yl)-2- fluorophenyl)-2- methyl-indoline- 1- sulfonamide | 770.2 |
| 141 | 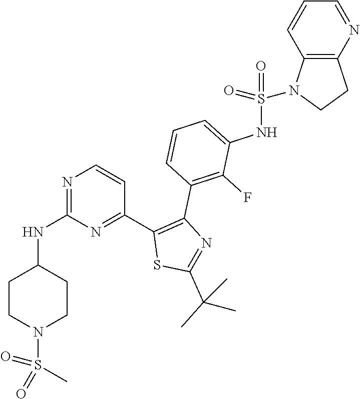 [SMILES: CC(C)(C)c1nc(-c2cccc(NS(=O)(=O)N3CCc4ncccc43)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2,3- dihydro- 1H-pyrrolo [3,2-b]- pyridine- 1-sulfonamide | 687.3 |
| 142 | 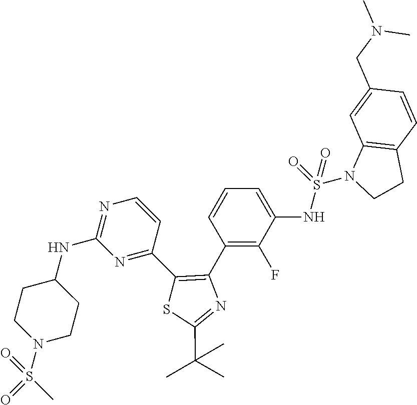 [SMILES: CN(C)Cc1ccc2c(c1)N(S(=O)(=O)Nc1cccc(-c3nc(C(C)(C)C)sc3-c3ccnc(NC4CCN(S(C)(=O)=O)CC4)n3)c1F)CC2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-6- ((dimethylamino)- methyl)- indoline- 1-sulfonamide | 743.3 |
| 143 | 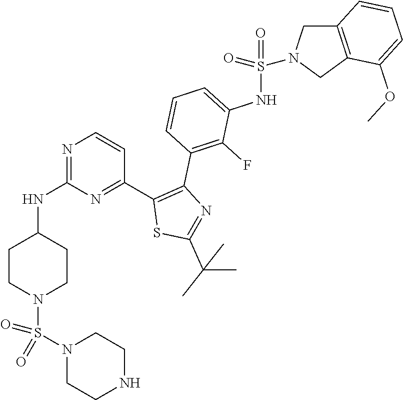 [SMILES: COc1cccc2c1CN(S(=O)(=O)Nc1cccc(-c3nc(C(C)(C)C)sc3-c3ccnc(NC4CCN(S(=O)(=O)N5CCNCC5)CC4)n3)c1F)C2] | N-(3-(2- (tert-butyl)- S-(2-((1- (piperazin- 1-ylsulfonyl) piperidin-4- yl)amino)- pyrimidin-4- yl)thiazol-4- yl)-2-fluoro- phenyl)-4- methoxy- isoindoline-2- sulfonamide | 786.4 |
| 144 | 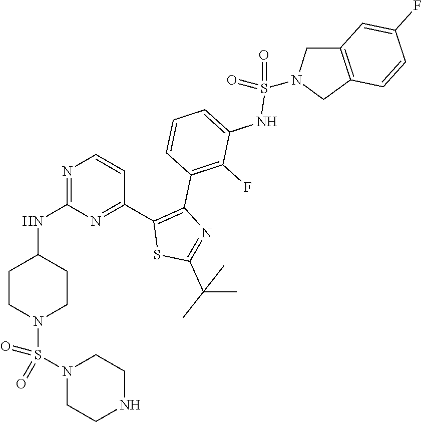 [SMILES: CC(C)(C)c1nc(-c2cccc(NS(=O)(=O)N3Cc4ccc(F)cc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-5- fluoro- isoindoline-2- sulfonamide | 774.2 |
| 145 | 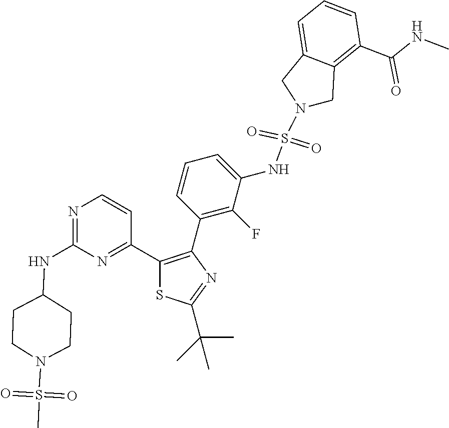 [SMILES: CNC(=O)c1cccc2c1CN(S(=O)(=O)Nc1cccc(-c3nc(C(C)(C)C)sc3-c3ccnc(NC4CCN(S(C)(=O)=O)CC4)n3)c1F)C2] | 2-(N-(3- (2-(tert-butyl)- 5-(2-((1- (methyl- sulfonyl) piperidin-4- yl)amino) pyrimidin-4- yl)thiazol-4- yl)-2- fluorophenyl)- sulfamoyl)-N- methyliso- indoline-4- carboxamide | 743.2 |
| 146 | 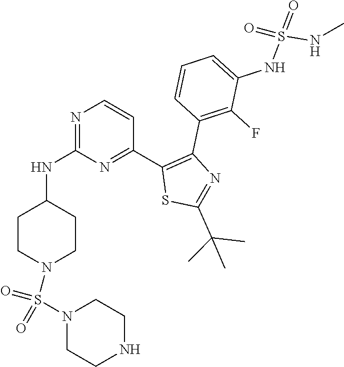 [SMILES: CNS(=O)(=O)Nc1cccc(-c2nc(C(C)(C)C)sc2-c2ccnc(NC3CCN(S(=O)(=O)N4CCNCC4)CC3)n2)c1F] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)- methylamine- sulfonamide | 668.2 |
| 147 | 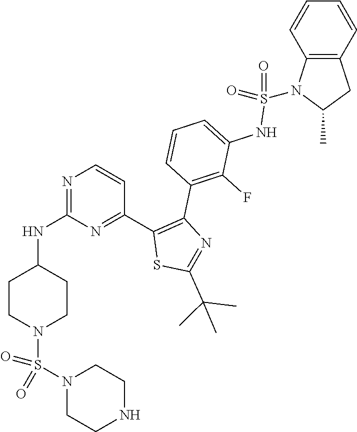 [SMILES: C[C@H]1Cc2ccccc2N1S(=O)(=O)Nc1cccc(-c2nc(C(C)(C)C)sc2-c2ccnc(NC3CCN(S(=O)(=O)N4CCNCC4)CC3)n2)c1F] | (S)-N-(3-(2- (tert-butyl)- 5-(2-((1- (piperazin- 1-ylsulfonyl) piperidin-4- yl)amino) pyrimidin-4- yl)thiazol-4- yl)-2- fluorophenyl)-2- methylindoline- 1-sulfonamide | 770.4 |
| 148 | 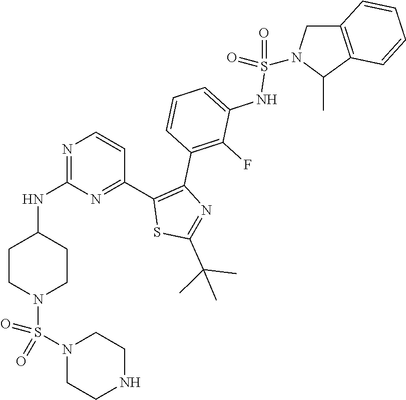 [SMILES: CC1c2ccccc2CN1S(=O)(=O)Nc1cccc(-c2nc(C(C)(C)C)sc2-c2ccnc(NC3CCN(S(=O)(=O)N4CCNCC4)CC3)n2)c1F] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-1- methylisoindoline- 2-sulfonamide | 770.4 |
| 149 | 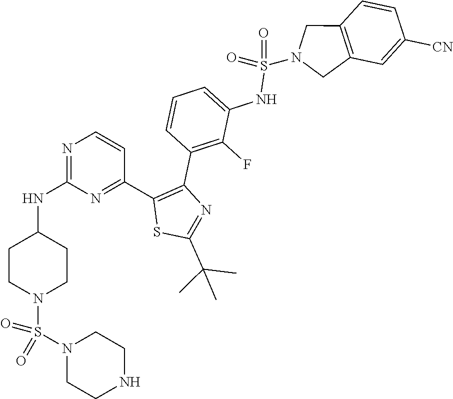 [SMILES: CC(C)(C)c1nc(-c2cccc(NS(=O)(=O)N3Cc4ccc(C#N)cc4C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-5- cyanoisoindoline- 2-sulfonamide | 781.2 |
| 150 | 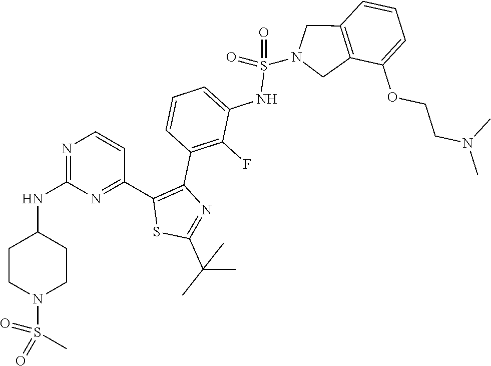 [SMILES: CN(C)CCOc1cccc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl) piperidin-4- yl)amino)- pyrimidin-4- yl)thiazol-4- yl)-2-fluoro- phenyl)-4-(2- (dimethylamino)- ethoxy)- isoindoline-2- sulfonamide | 773.4 |
| 151 | 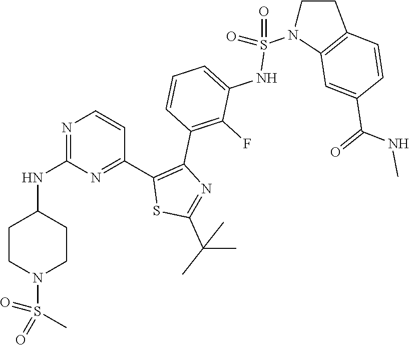 [SMILES: CNC(=O)c1ccc2c(c1)N(S(=O)(=O)Nc1cccc(-c3nc(C(C)(C)C)sc3-c3ccnc(NC4CCN(S(C)(=O)=O)CC4)n3)c1F)CC2] | 1-(N-(3-(2- (tert-butyl)- 5-(2-((1- (methyl- sulfonyl) piperidin-4- yl)amino) pyrimidin- 4-yl)thiazol-4- yl)-2- fluorophenyl)- sulfamoyl)-N- methylindoline- 6-carboxamide | 743.4 |
| 152 | 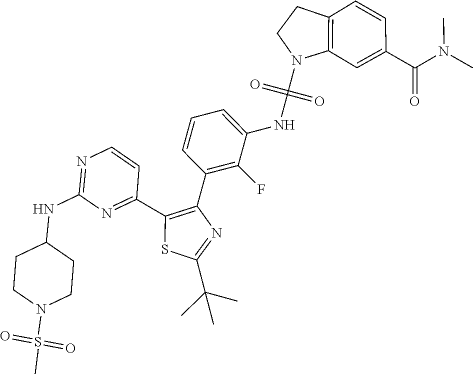 | 1-(N-(3-(2- (tert-butyl)- 5-(2-((1- (methyl- sulfonyl) piperidin-4- yl)amino) pyrimidin- 4-yl)thiazol-4- yl)-2- fluorophenyl)- sulfamoyl)-N,N- dimethylindoline- 6-carboxamide | 757.4 |
| 153 | 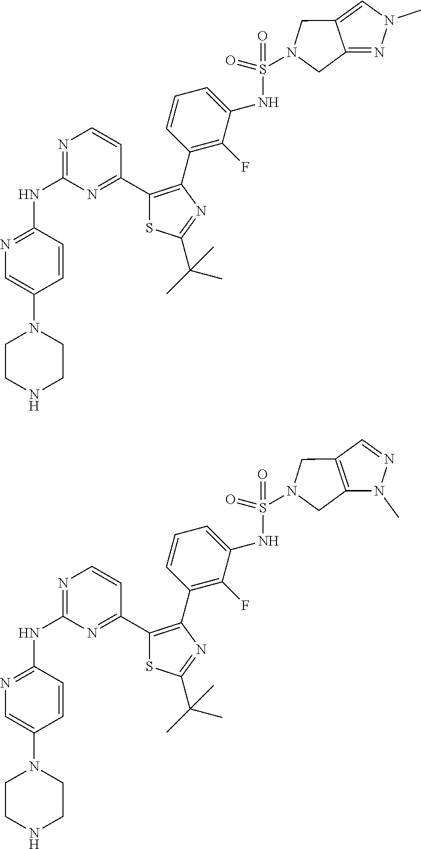 [SMILES: Cn1cc2c(n1)CN(S(=O)(=O)Nc1cccc(-c3nc(C(C)(C)C)sc3-c3ccnc(Nc4ccc(N5CCNCC5)cn4)n3)c1F)C2 ; Cn1ncc2c1CN(S(=O)(=O)Nc1cccc(-c3nc(C(C)(C)C)sc3-c3ccnc(Nc4ccc(N5CCNCC5)cn4)n3)c1F)C2] | a mixture of N- (3-(2-(tert-butyl)- 5-(2- ((5-(piperazin- 1-yl)pyridin-2- yl)amino)pyrimidin- 4-yl)thiazol-4- yl)-2- fluorophenyl)-2- methyl-2,6- dihydropyrrolo [3,4-c]pyrazole- 5(4H)- sulfonamide and N-(3-(2-(tert-butyl)- 5-(2-((5-(piperazin- 1-yl)pyridin-2-yl) amino)pyrimidin-4- yl)thiazol-4- yl)-2-fluorophenyl)- 1-methyl-4,6- dihydropyrrolo [3,4-c]- pyrazole-5(1H)- sulfonamide | 690.3 |
| 154 | 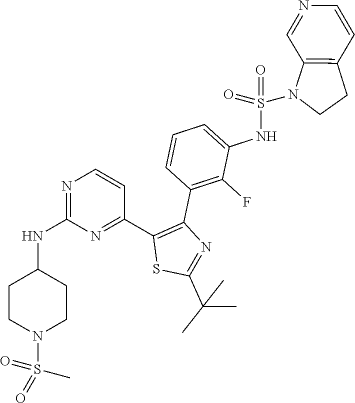 [SMILES: CC(C)(C)c1nc(-c2cccc(NS(=O)(=O)N3CCc4ccncc43)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2,3-dihydro- 1H-pyrrolo[2,3-c]- pyridine- 1-sulfonamide | 687.3 |
| 155 | 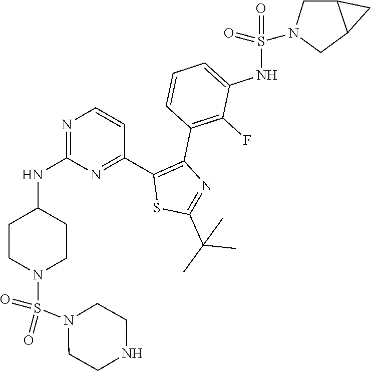 [SMILES: CC(C)(C)c1nc(-c2cccc(NS(=O)(=O)N3CC4CC4C3)c2F)c(-c2ccnc(NC3CCN(S(=O)(=O)N4CCNCC4)CC3)n2)s1] | N-(3-(2-(tert-butyl)- 5-(2-((1-(piperazin- 1-ylsulfonyl) piperidin-4- yl)amino)- pyrimidin-4- yl)thiazol-4- yl)-2- fluorophenyl)-3- azabicyclo[3.1.0]- hexane-3-sulfonamide | 720.3 |
| 156 | 1 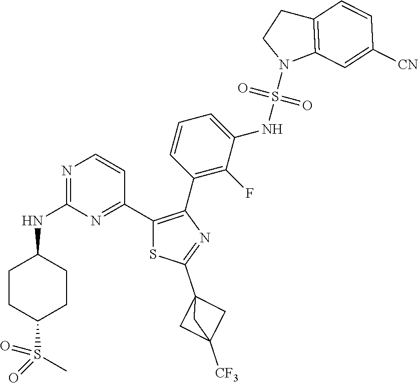 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(C#N)cc54)c3F)n2)CC1] | N-(3-(2-(tert-butyl)- 5-(2-((1-(N-(2- (dimethylamino) ethyl)-N- methyl- sulfamoyl) piperidin-4- yl)amino)- pyrimidin-4- yl)thiazol-4- yl)-2-fluoro- phenyl)isoindoline- 2-sulfonamide | 772.5 |
| 157 | 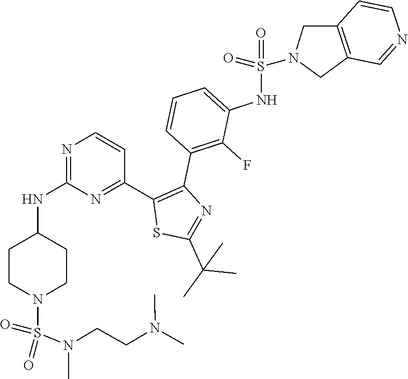 [SMILES: CN(C)CCN(C)S(=O)(=O)N1CCC(Nc2nccc(-c3sc(C(C)(C)C)nc3-c3cccc(NS(=O)(=O)N4Cc5ccncc5C4)c3F)n2)CC1] | N-(3-(2- (tert-butyl)- 5-(2-((1-(N-(2- (dimethyl-amino) ethyl)-N- methylsulfamoyl) piperidin-4- yl)amino)- pyrimidin-4- yl)thiazol-4- yl)-2-fluoro- phenyl)-1, 3-dihydro- 2H-pyrrolo [3,4-c]- pyridine-2- sulfonamide | 773.4 |
| 158 | 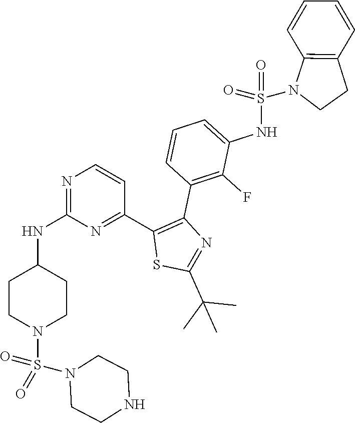 [SMILES: CC(C)(C)c1nc(-c2cccc(NS(=O)(=O)N3CCc4ccccc43)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)indoline- 1-sulfonamide | 756.4 |
| 159 | 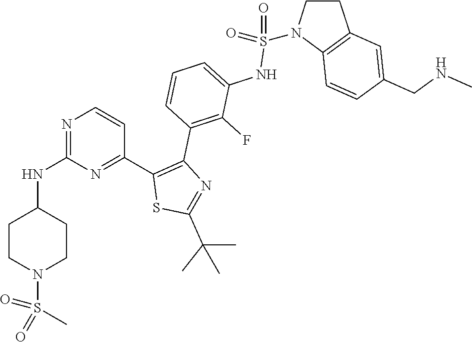 [SMILES: CNCc1ccc2c(c1)CCN2S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- ((methylamino) methyl)- indoline- 1-sulfonamide | 729.3 |
| 160 | 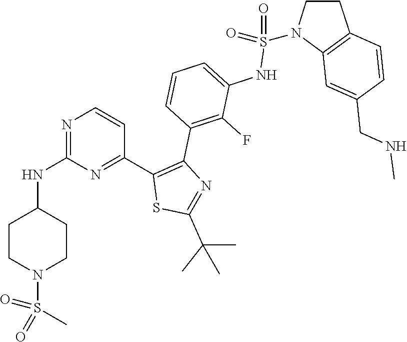 [SMILES: CNCc1ccc2c(c1)N(S(=O)(=O)Nc1cccc(-c3nc(C(C)(C)C)sc3-c3ccnc(NC4CCN(S(C)(=O)=O)CC4)n3)c1F)CC2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-6- ((methylamino) methyl)- indoline- 1-sulfonamide | 729.3 |
| 161 | 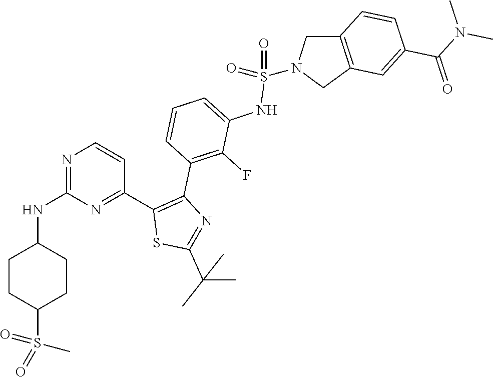 [SMILES: CN(C)C(=O)c1ccc2c(c1)CN(S(=O)(=O)Nc1cccc(-c3nc(C(C)(C)C)sc3-c3ccnc(NC4CCC(S(C)(=O)=O)CC4)n3)c1F)C2] | 2-(N-(3-(2- (tert-butyl)- 5-(2-((1- (methyl- sulfonyl) piperidin-4- yl)amino) pyrimidin- 4~yl)thiazol~4- yl)-2- fluorophenyl)- sulfamoyl)- N,N- dimethyl- isoindoline-5- carboxamide | 757.1 |
| 162 | 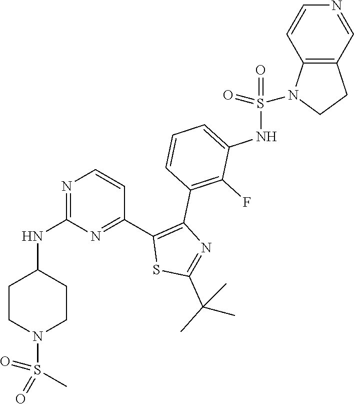 [SMILES: CC(C)(C)c1nc(-c2cccc(NS(=O)(=O)N3CCc4cnccc43)c2F)c(-c2ccnc(NC3CCN(S(C)(=O)=O)CC3)n2)s1] | N-(3-(2- (tert-butyl)- 5-(2-((1- (methylsulfonyl) piperidin-4- yl)amino)- pyrimidin-4- yl)thiazol-4- yl)-2-fluoro- phenyl)-2,3- dihydro- 1H-pyrrolo [3,2-c]- pyridine- 1-sulfonamide | 687.1 |
| 163 | 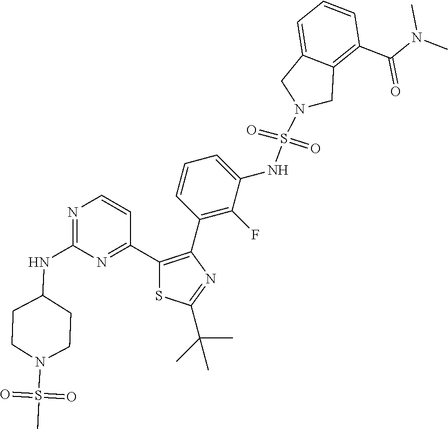 [SMILES: CN(C)C(=O)c1cccc2c1CN(S(=O)(=O)Nc1cccc(-c3nc(C(C)(C)C)sc3-c3ccnc(NC4CCN(S(C)(=O)=O)CC4)n3)c1F)C2] | 2-(N-(3-(2- (tert-butyl)- 5-(2-((1- (methyl- sulfonyl) piperidin-4- yl)amino) pyrimidin-4- yl)thiazol-4- yl)-2- fluorophenyl)- sulfamoyl)-N, N-dimethyliso- indoline-4- carboxamide | 757.3 |
| 164 | 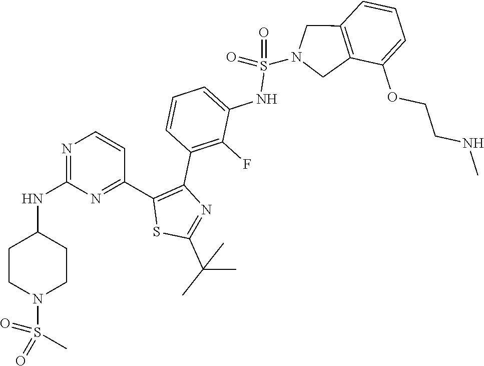 [SMILES: CNCCOc1cccc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4-(2- (methylamino)- ethoxy)- isoindoline-2- sulfonamide | 759.4 |
| 165 | 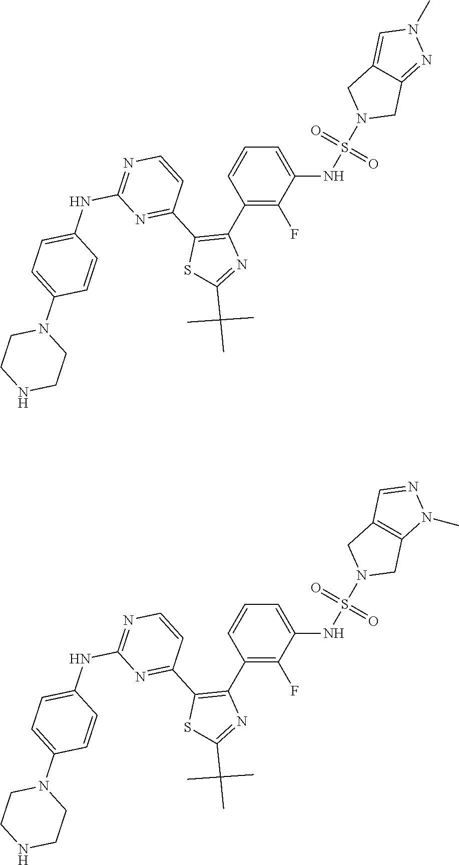 [SMILES: Cn1cc2c(n1)CN(S(=O)(=O)Nc1cccc(-c3nc(C(C)(C)C)sc3-c3ccnc(Nc4ccc(N5CCNCC5)cc4)n3)c1F)C2 ; Cn1ncc2c1CN(S(=O)(=O)Nc1cccc(-c3nc(C(C)(C)C)sc3-c3ccnc(Nc4ccc(N5CCNCC5)cc4)n3)c1F)C2] | a mixture of N-(3-(2-(tert-butyl)- 5-(2- ((4-(piperazin- 1-yl)phenyl)amino)- pyrimidin-4- yl)thiazol-4- yl)-2-fluoro- phenyl)-2- methyl-2,6- dihydropyrrolo- [3,4-c]pyrazole- 5(4H)-sulfonamide and N-(3-(2-(tert-butyl)- 5-(2-((4-(piperazin- 1-yl)phenyl)amino) pyrimidin-4~ yl)thiazol-4- yl)-2-fluorophenyl)- 1-methyl-4,6- dihydropyrrolo- [3,4-c]- pyrazole-5(1H)- sulfonamide | 689.2 |
| 166 | 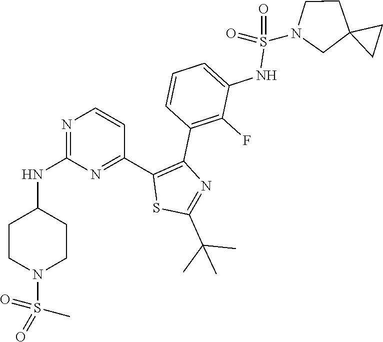 [SMILES: CC(C)(C)c1nc(-c2cccc(NS(=O)(=O)N3CCC4(CC4)C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- azaspiro[2.4] heptane-5- sulfonamide | 664.3 |
| 167 | 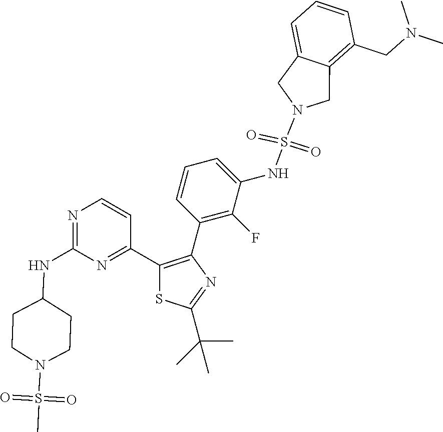 [SMILES: CN(C)Cc1cccc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- ((dimethylamino)- methyl)- isoindoline-2- sulfonamide | 743.4 |
| 168 | 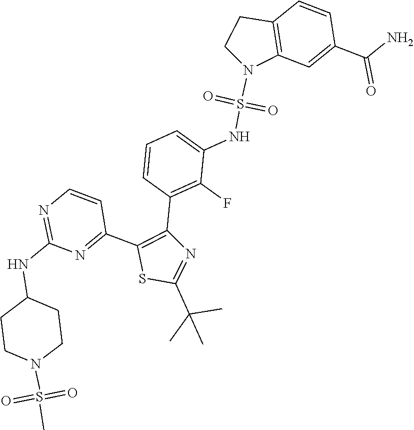 [SMILES: CC(C)(C)c1nc(-c2cccc(NS(=O)(=O)N3CCc4ccc(C(N)=O)cc43)c2F)c(-c2ccnc(NC3CCN(S(C)(=O)=O)CC3)n2)s1] | 1-(N-(3-(2- (tert-butyl)- 5-(2-((1-(methyl- sulfonyl) piperidin-4- yl)amino) pyrimidin-4- yl)thiazol-4- yl)-2-fluorophenyl)- sulfamoyl)- indoline-6- carboxamide | 729.3 |
| 169 | 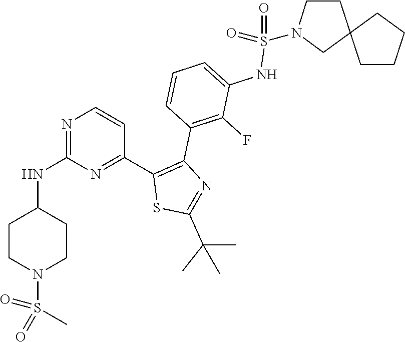 [SMILES: CC(C)(C)c1nc(-c2cccc(NS(=O)(=O)N3CCC4(CCCC4)C3)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-2-azaspiro [4.4]nonane-2- sulfonamide | 692.4 |
| 170 | 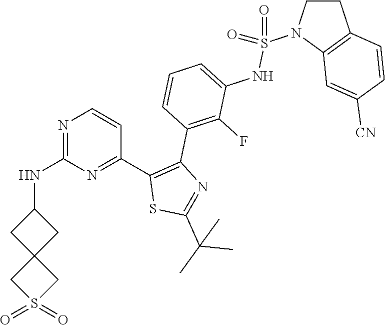 [SMILES: CC(C)(C)c1nc(-c2cccc(NS(=O)(=O)N3CCc4ccc(C#N)cc43)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6- yl)amino)- pyrimidin-4- yl)thiazol-4- yl)-2-fluoro- phenyl)-6- cyano-indoline- 1-sulfonamide | 694.2 |
| 171 | 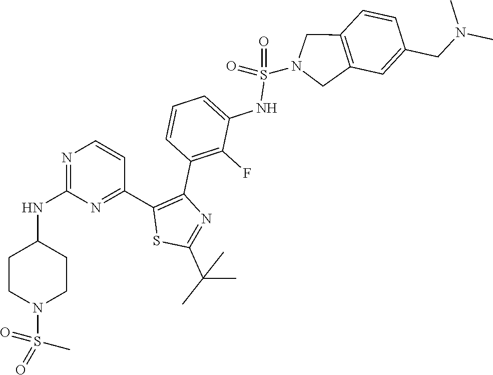 [SMILES: CN(C)Cc1ccc2c(c1)CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- ((dimethylamino) methyl)- isoindoline-2- sulfonamide | 743.4 |
| 172 | 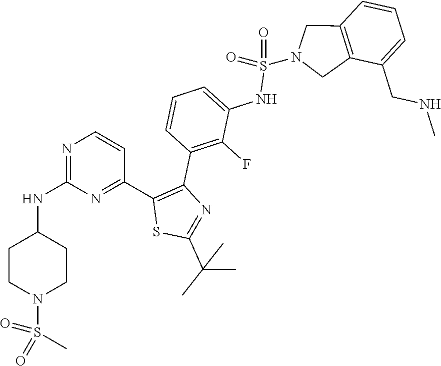 [SMILES: CNCc1cccc2c1CN(S(=O)(=O)Nc1cccc(-c3nc(C(C)(C)C)sc3-c3ccnc(NC4CCN(S(C)(=O)=O)CC4)n3)c1F)C2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- ((methylamino) methyl)- isoindoline-2- sulfonamide | 728.9 |
| 173 | 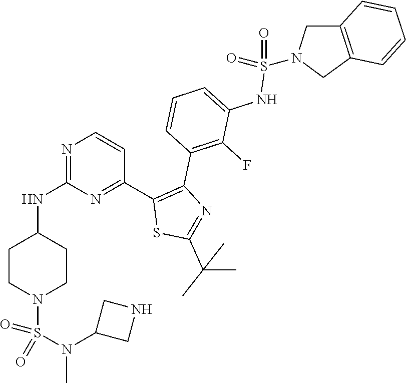 [SMILES: CN(C1CNC1)S(=O)(=O)N1CCC(Nc2nccc(-c3sc(C(C)(C)C)nc3-c3cccc(NS(=O)(=O)N4Cc5ccccc5C4)c3F)n2)CC1] | N-(3-(5-(2-((1- (N-(azetidin- 3-yl)-N- methylsulfamoyl) piperidin-4- yl)amino)- pyrimidin-4- yl)-2-(tert- butyl)thiazol-4- yl)-2-fluorophenyl) isoindoline-2- sulfonamide | 756.3 |
| 174 | 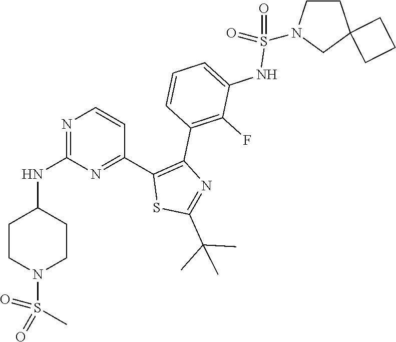 [SMILES: CC(C)(C)c1nc(-c2cccc(NS(=O)(=O)N3CCC4(CCC4)C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-6-azaspiro [3.4]octane-6- sulfonamide | 677.9 |
| 175 | 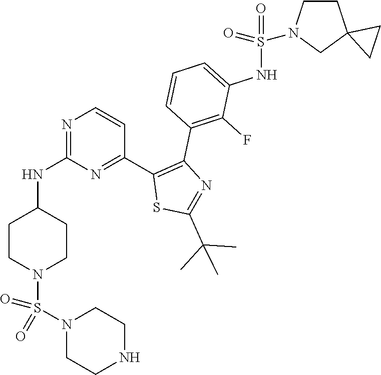 [SMILES: CC(C)(C)c1nc(-c2cccc(NS(=O)(=O)N3CCC4(CC4)C3)c2F)c(-c2ccnc(NC3CCN(S(=O)(=O)N4CCNCC4)CC3)n2)s1] | N-(3-(2-(tert-butyl)- 5-(2-((1-(piperazin- 1-ylsulfonyl) piperidin-4- yl)amino)- pyrimidin-4- yl)thiazol-4- yl)-2-fluoro- phenyl)-5-azaspiro [2.4]heptane-5- sulfonamide | 733.9 |
| 176 | 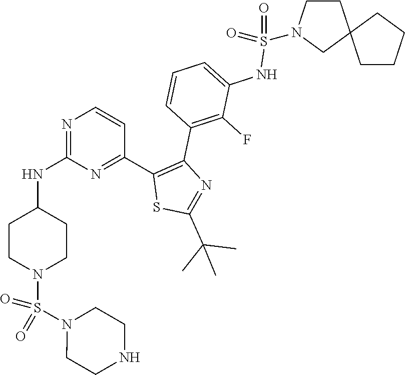 [SMILES: CC(C)(C)c1nc(-c2cccc(NS(=O)(=O)N3CCC4(CCCC4)C3)c2F)c(-c2ccnc(NC3CCN(S(=O)(=O)N4CCNCC4)CC3)n2)s1] | N-(3-(2- (tert-butyl)- 5-(2-((1- (piperazin- 1-ylsulfonyl) piperidin-4- yl)amino)- pyrimidin-4- yl)thiazol-4- yl)-2-fluoro- phenyl)-2- azaspiro[4.4] nonane-2- sulfonamide | 761.9 |
| 177 | 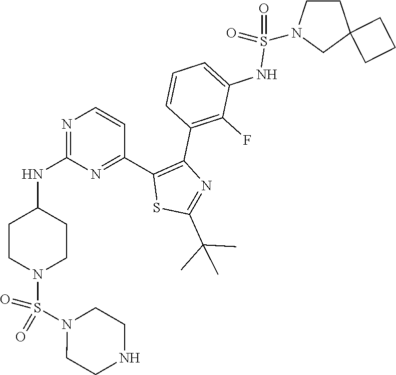 [SMILES: CC(C)(C)c1nc(-c2cccc(NS(=O)(=O)N3CCC4(CCC4)C3)c2F)c(-c2ccnc(NC3CCN(S(=O)(=O)N4CCNCC4)CC3)n2)s1] | N-(3-(2- (tert-butyl)- S-(2-((1- (piperazin- 1-ylsulfonyl) piperidin-4- yl)amino)- pyrimidin-4- yl)thiazol-4- yl)-2-fluoro- phenyl)-6- azaspiro[3.4] octane-6- sulfonamide | 747.9 |
| 178 | 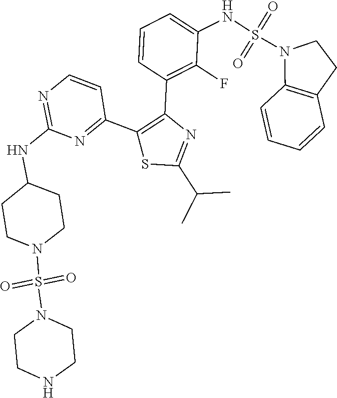 [SMILES: CC(C)c1nc(-c2cccc(NS(=O)(=O)N3CCc4ccccc43)c2F)c(-c2ccnc(NC3CCN(S(=O)(=O)N4CCNCC4)CC3)n2)s1] | N-(2-fluoro-3- (2-isopropyl- 5-(2-((1- (piperazin- 1-ylsulfonyl) piperidin-4- yl)amino) pyrimidin-4- yl)thiazol-4- yl)phenyl)indoline- 1-sulfonamide | 742.3 |
| 179 | 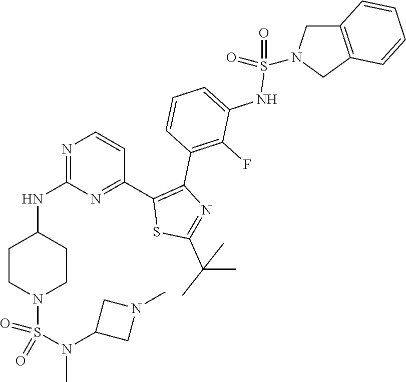 [SMILES: CN1CC(N(C)S(=O)(=O)N2CCC(Nc3nccc(-c4sc(C(C)(C)C)nc4-c4cccc(NS(=O)(=O)N5Cc6ccccc6C5)c4F)n3)CC2)C1] | N-(3-(2- (tert-butyl)- 5-(2-((1-(N- methyl- N-(1- methylazetidin- 3-yl)sulfamoyl)- piperidin-4- yl)amino) pyrimidin-4- yl)-thiazol-4- yl)-2-fluorophenyl)- isoindoline- 2-sulfonamide | 770.3 |
| 180 | 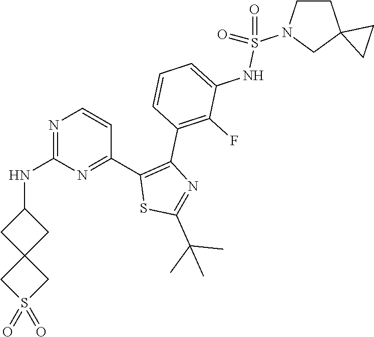 [SMILES: CC(C)(C)c1nc(-c2cccc(NS(=O)(=O)N3CCC4(CC4)C3)c2F)c(-c2ccnc(NC3CC4(C3)CS(=O)(=O)C4)n2)s1] | N-(3-(2- (tert-butyl)- 5-(2-((2, 2-dioxido-2- thiaspiro[3.3] heptan-6-yl)amino)- pyrimidin-4- yl)thiazol-4- yl)-2-fluoro- phenyl)-5- azaspiro[2.4] heptane-5- sulfonamide | 647.3 |
| 181 | 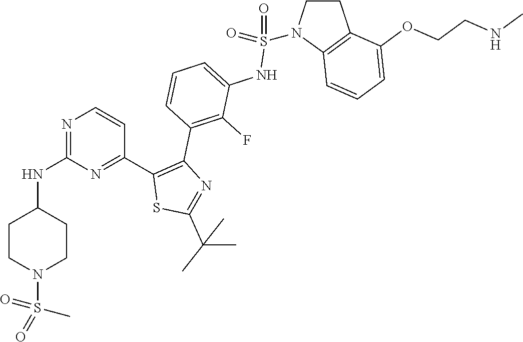 [SMILES: CNCCOc1cccc2c1CCN2S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyrimidin-4- yl)thiazol-4- yl)-2-fluoro- phenyl)-4- (2-(methylamino)- ethoxy)- indoline- 1-sulfonamide | 759.4 |
| 182 | 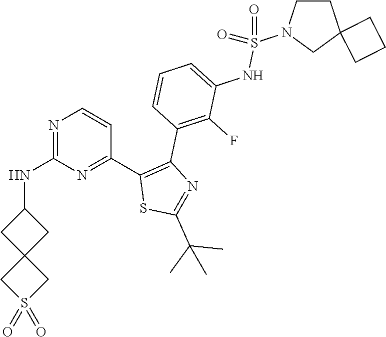 [SMILES: CC(C)(C)c1nc(-c2cccc(NS(=O)(=O)N3CCC4(CCC4)C3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl) amino)- pyrimidin-4- yl)thiazol-4- yl)-2-fluoro- phenyl)-6- azaspiro[3.4] octane-6- sulfonamide | 661.2 |
| 183 | 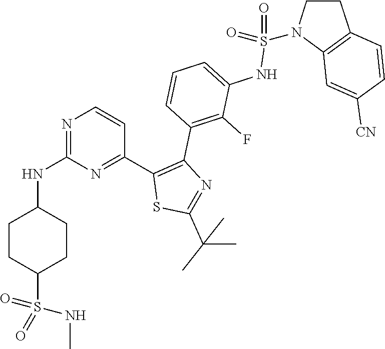 [SMILES: CNS(=O)(=O)C1CCC(Nc2nccc(-c3sc(C(C)(C)C)nc3-c3cccc(NS(=O)(=O)N4CCc5ccc(C#N)cc54)c3F)n2)CC1] | N-(3-(2- (tert-butyl)- 5-(2-((1-(N- methyl- sulfamoyl) piperidin-4- yl)amino)- pyrimidin-4- yl)thiazol-4- yl)-2~fluoro- phenyl)-6- cyano-indoline- 1-sulfonamide | 726.3 |
| 184 | 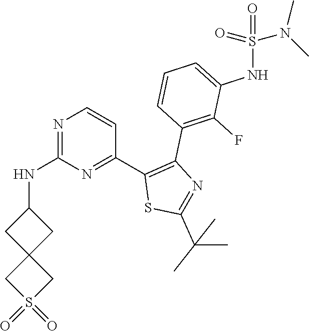 [SMILES: CN(C)S(=O)(=O)Nc1cccc(-c2nc(C(C)(C)C)sc2-c2ccnc(NC3CC4(C3)CS(=O)(=O)C4)n2)c1F] | N-(3-(2-(tert-butyl)- 5-(2-((2,2- dioxido-2- thiaspiro[3.3] heptan-6-yl)amino)- pyrimidin-4- yl)thiazol-4- yl)-2-fluoro- phenyl)dimethyl- aminesulfonamide | 595.2 |
| 185 | 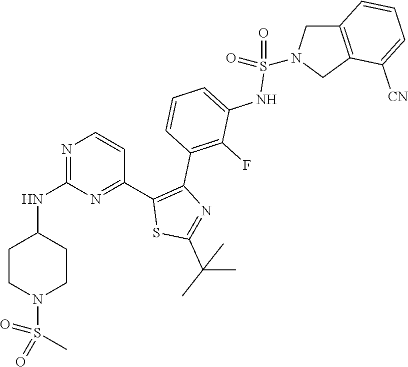 [SMILES: CC(C)(C)c1nc(-c2cccc(NS(=O)(=O)N3Cc4cccc(C#N)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol- 4-yl)-2-fluoro- phenyl)-4- cyanoisoindoline-2- sulfonamide | 711.2 |
| 186 | 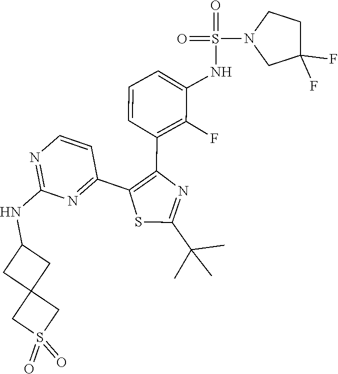 [SMILES: CC(C)(C)c1nc(-c2cccc(NS(=O)(=O)N3CCC(F)(F)C3)c2F)c(-c2ccnc(NC3CC4(C3)CS(=O)(=O)C4)n2)s1] | N-(3-(2-(tert-butyl)- 5-(2-((2,2- dioxido-2- thiaspiro[3.3] heptan-6-yl)amino)- pyrimidin-4- yl)thiazol-4- yl)-2-fluoro- phenyl)-3,3- difluoropyrrolidine- 1-sulfonamide | 657.2 |
| 187 | 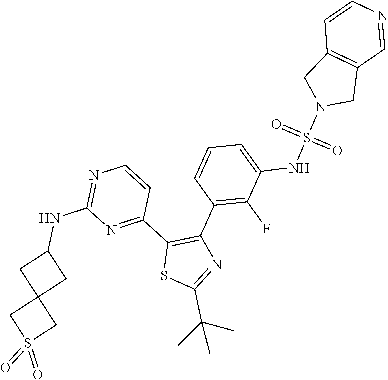 [SMILES: CC(C)(C)c1nc(-c2cccc(NS(=O)(=O)N3Cc4ccncc4C3)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-1,3- dihydro-2H- pyrrolo[3,4-c]- pyridine-2- sulfonamide | 670.2 |
| 188 | 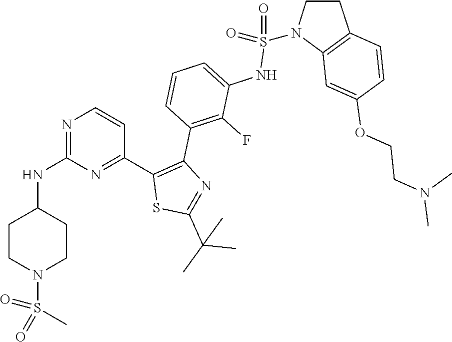 [SMILES: CN(C)CCOc1ccc2c(c1)N(S(=O)(=O)Nc1cccc(-c3nc(C(C)(C)C)sc3-c3ccnc(NC4CCN(S(C)(=O)=O)CC4)n3)c1F)CC2] | N-(3-(2- (tert-butyl)- 5-(2-((1- (methylsulfonyl)- piperidin-4- yl)amino)- pyrimidin-4- yl)thiazol-4- yl)-2-fluoro- phenyl)-6-(2- (dimethylamino)- ethoxy)- indoline- 1-sulfonamide | 773.4 |
| 189 | 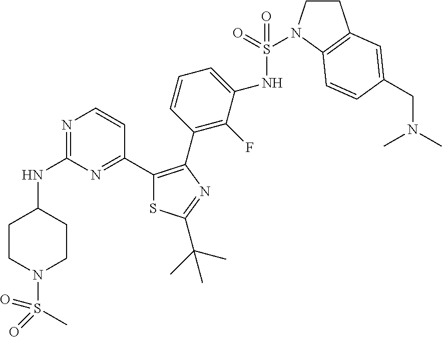 [SMILES: CN(C)Cc1ccc2c(c1)CCN2S(=O)(=O)Nc1cccc(-c2nc(C(C)(C)C)sc2-c2ccnc(NC3CCN(S(C)(=O)=O)CC3)n2)c1F] | N-(3-(2- (tert-butyl)- 5-(2-((1- (methylsulfonyl)- piperidin-4- yl)amino)- pyrimidin-4- yl)thiazol-4- yl)-2-fluoro- phenyl)-5- ((dimethylamino)- methyl)- indoline- 1-sulfonamide | 743.3 |
| 190 | 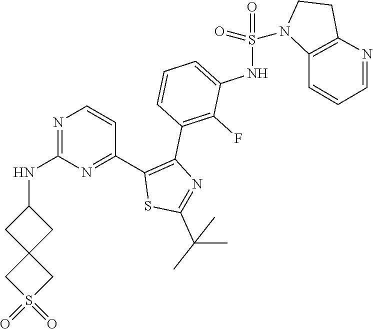 [SMILES: CC(C)(C)c1nc(-c2cccc(NS(=O)(=O)N3CCc4ncccc43)c2F)c(-c2ccnc(NC3CC4(C3)CS(=O)(=O)C4)n2)s1] | N-(3-(2-(tert-butyl)- 5-(2-((2,2- dioxido-2- thiaspiro[3.3] heptan-6-yl)amino)- pyrimidin-4- yl)thiazol-4- yl)-2-fluoro- phenyl)-2,3-dihydro- 1H-pyrrolo[3,2-b]- pyridine- 1-sulfonamide | 670.2 |
| 191 | 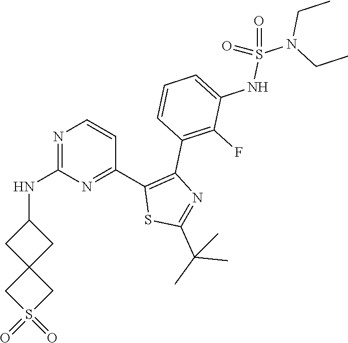 [SMILES: CCN(CC)S(=O)(=O)Nc1cccc(-c2nc(C(C)(C)C)sc2-c2ccnc(NC3CC4(C3)CS(=O)(=O)C4)n2)c1F] | N-(3-(2-(tert-butyl)- 5-(2-((2,2-dioxido- 2-thiaspiro[3.3] heptan-6-yl)amino)- pyrimidin-4- yl)thiazol-4- yl)-2-fluoro- phenyl)-diethylamine- sulfonamide | 623.2 |
| 192 | 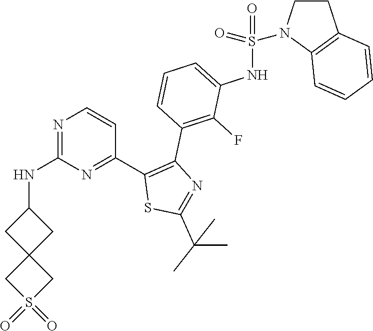 [SMILES: CC(C)(C)c1nc(-c2cccc(NS(=O)(=O)N3CCc4ccccc43)c2F)c(-c2ccnc(NC3CC4(C3)CS(=O)(=O)C4)n2)s1] | N-(3-(2- (tert-butyl)- 5-(2-((2,2-dioxido-2- thiaspiro[3.3] heptan-6-yl)amino)- pyrimidin-4- yl)thiazol-4- yl)-2-fluoro- phenyl)indoline- 1-sulfonamide | 669.3 |
| 193 | 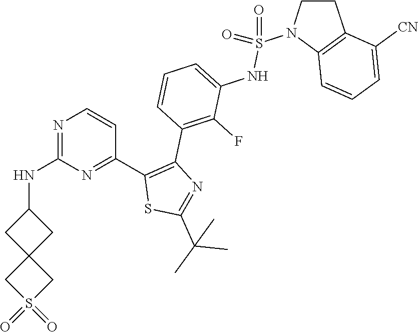 [SMILES: CC(C)(C)c1nc(-c2cccc(NS(=O)(=O)N3CCc4c(C#N)cccc43)c2F)c(-c2ccnc(NC3CC4(C3)CS(=O)(=O)C4)n2)s1] | N-(3-(2-(tert-butyl)- 5-(2-((2,2- dioxido-2- thiaspiro[3.3] heptan-6-yl)amino)- pyrimidin-4- yl)thiazol-4- yl)-2-fluoro- phenyl)-4- cyanoindoline- 1-sulfonamide | 694.2 |
| 194 | 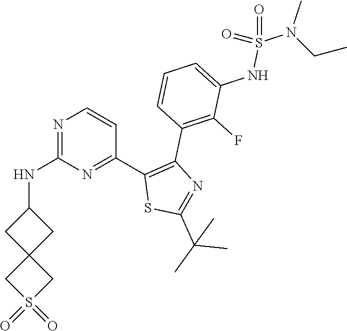 [SMILES: CCN(C)S(=O)(=O)Nc1cccc(-c2nc(C(C)(C)C)sc2-c2ccnc(NC3CC4(C3)CS(=O)(=O)C4)n2)c1F] | N-(3-(2- (tert-butyl)- 5-(2-((2,2- dioxido-2- thiaspiro[3.3] heptan-6- yl)amino)- pyrimidin-4- yl)thiazol-4- yl)-2-fluoro- phenyl)-N- methyl-N- ethylamine- sulfonamide | 609.3 |
| 195 | 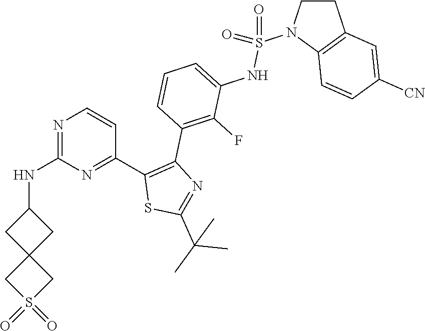 [SMILES: CC(C)(C)c1nc(-c2cccc(NS(=O)(=O)N3CCc4cc(C#N)ccc43)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6- yl)amino)- pyrimidin-4- yl)thiazol-4- yl)-2-fluoro- phenyl)-5- cyanoindoline- 1-sulfonamide | 694.3 |
| 196 | 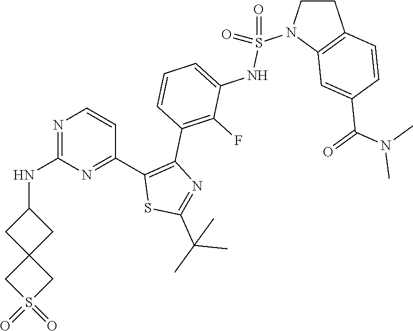 [SMILES: CN(C)C(=O)c1ccc2c(c1)N(S(=O)(=O)Nc1cccc(-c3nc(C(C)(C)C)sc3-c3ccnc(NC4CC5(C4)CS(=O)(=O)C5)n3)c1F)CC2] | 2-(N-(3-(2- (tert-butyl)- 5-(2-((2,2- dioxido-2- thiaspiro[3.3] heptan-6-yl)- amino) pyrimidin-4- yl)thiazol- 4-yl)-2- fluorophenyl) sulfamoyl)-N,N- dimethylisoindoline- 4-carboxamide | 740.2 |
| 197 | 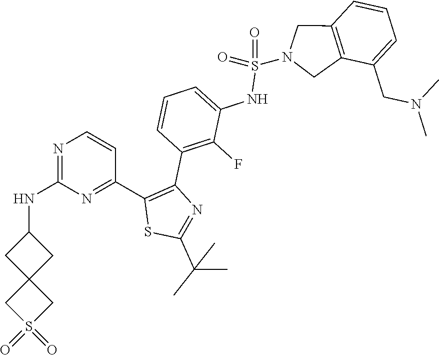 [SMILES: CN(C)Cc1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-4- ((dimethylamino) methyl)- isoindoline- 2-sulfonamide | 726.3 |
| 198 | 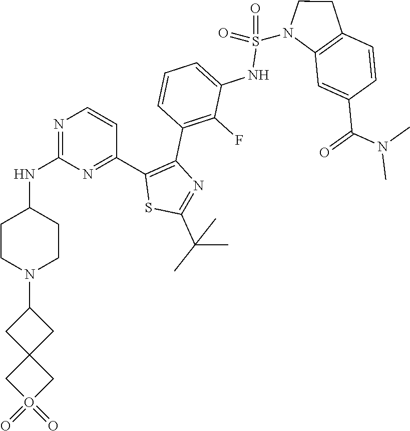 | 1-(N-(3-(2- (tert-butyl)- 5-(2-((2,2- dioxido-2- thiaspiro[3.3] heptan-6- yl)amino) pyrimidin-4- yl)thiazol-4- yl)-2- fluorophenyl) sulfamoyl)-N,N- dimethylindoline- 6-carboxamide | 740.3 |
| 199 | 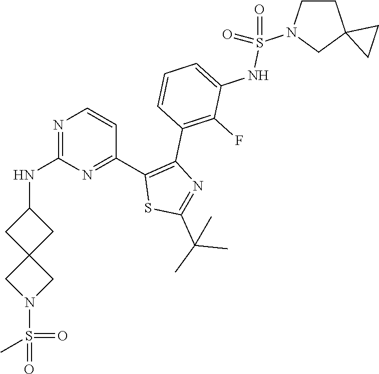 [SMILES: CC(C)(C)c1nc(-c2cccc(NS(=O)(=O)N3CCC4(CC4)C3)c2F)c(-c2ccnc(NC3CC4(C3)CN(S(C)(=O)=O)C4)n2)s1] | N-(3-(2- (tert-butyl)- 5-(2-((2- (methylsulfonyl)- 2-azaspiro [3.3]heptan- 6-yl)amino) pyrimidin-4- yl)thiazol-4-yl)- 2-fluorophenyl)- 5-azaspiro [2.4]heptane- 5-sulfonamide | 676.3 |
| 200 | 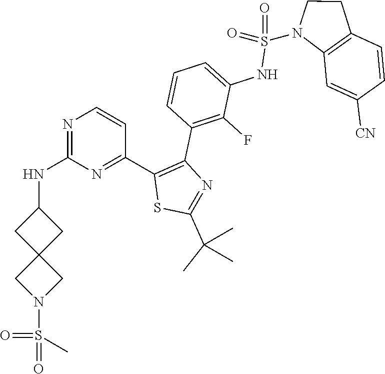 [SMILES: CC(C)(C)c1nc(-c2cccc(NS(=O)(=O)N3CCc4ccc(C#N)cc43)c2F)c(-c2ccnc(NC3CC4(C3)CN(S(C)(=O)=O)C4)n2)s1] | N-(3-(2-(tert- butyl)-5-(2-((2- (methylsulfonyl)- 2-azaspiro [3.3]heptan- 6-yl)amino) pyrimidin-4- yl)thiazol-4- yl)- 2-fluorophenyl)- 6-cyanoindoline- 1-sulfonamide | 723.2 |
| 201 | 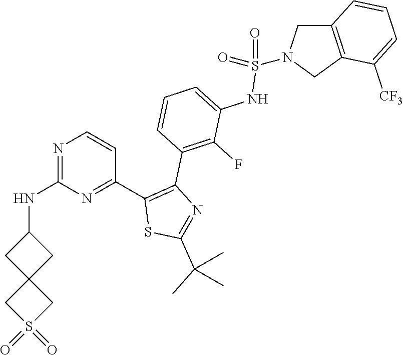 [SMILES: CC(C)(C)c1nc(-c2cccc(NS(=O)(=O)N3Cc4cccc(C(F)(F)F)c4C3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl)amino)- pyrimidin-4- yl)thiazol-4- yl)-2- fluorophenyl)-4- (trifluoromethyl)- isoindoline-2- sulfonamide | 737.2 |
| 202 | 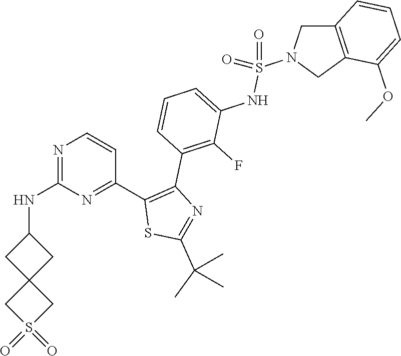 [SMILES: COc1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2- (tert-butyl)- S-(2-((2,2- dioxido-2- thiaspiro[3.3] heptan-6-yl)amino)- pyrimidin-4- yl)thiazol-4- yl)-2-fluoro- phenyl)-4- methoxyisoindoline-2- sulfonamide | 699.2 |
| 203 | 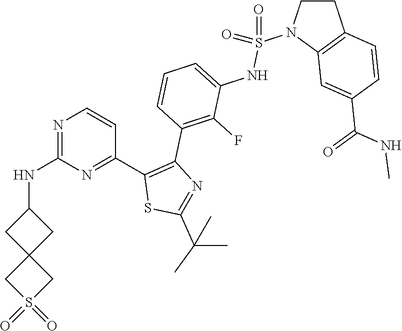 [SMILES: CNC(=O)c1ccc2c(c1)N(S(=O)(=O)Nc1cccc(-c3nc(C(C)(C)C)sc3-c3ccnc(NC4CC5(C4)CS(=O)(=O)C5)n3)c1F)CC2] | 1-(N-(3-(2- (tert-butyl)- 5-(2-((2,2- dioxido-2- thiaspiro[3.3] heptan-6- yl)amino) pyrimidin-4- yl)thiazol-4- yl)-2- fluorophenyl) sulfamoyl)-N- methyl- indoline-6- carboxamide | 726.3 |
| 204 | 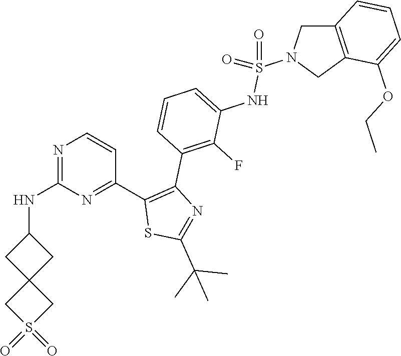 [SMILES: CCOc1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2- (tert-butyl)- 5-(2-((2,2- dioxido-2- thiaspiro[3.3] heptan-6- yl)amino)- pyrimidin-4- yl)thiazol-4- yl)-2-fluoro- phenyl)-4- ethoxyisoindoline- 2-sulfonamide | 713.2 |
| 205 | 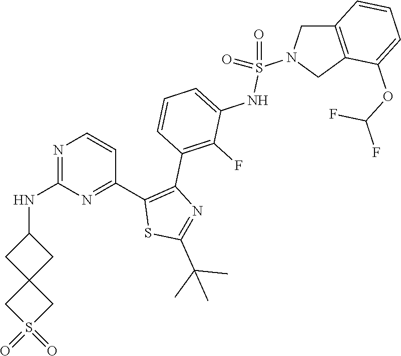 [SMILES: CC(C)(C)c1nc(-c2cccc(NS(=O)(=O)N3Cc4cccc(OC(F)F)c4C3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6- yl)amino)- pyrimidin-4- yl)thiazol-4- yl)-2- fluorophenyl)-4- (difluoromethoxy)- isoindoline- 2-sulfonamide | 735.2 |
| 206 | 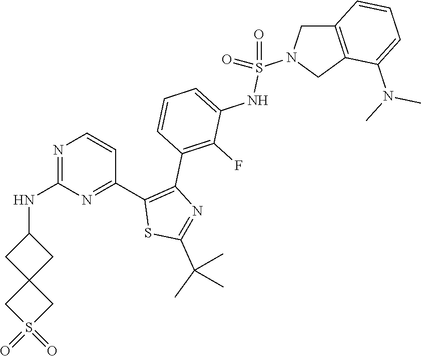 [SMILES: CN(C)c1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2-(tert-butyl)- S-(2-((2,2-dioxido-2- thiaspiro[3.3] heptan-6- yl)amino)- pyrimidin-4- yl)thiazol-4- yl)-2-fluoro- phenyl)-4- (dimethylamino) isoindoline-2- sulfonamide | 712.2 |
| 207 | 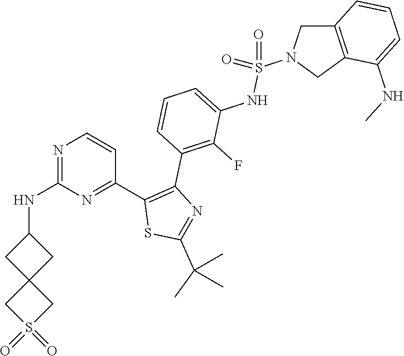 [SMILES: CNc1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2-(tert-butyl)- 5-(2-((2,2-dioxido-2- thiaspiro[3.3] heptan-6-yl)amino)- pyrimidin-4- yl)thiazol-4- yl)-2- fluorophenyl)-4- (methylamino)- isoindoline-2- sulfonamide | 698.2 |
| 208 | 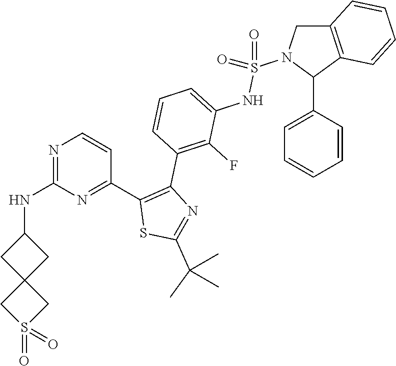 [SMILES: CC(C)(C)c1nc(-c2cccc(NS(=O)(=O)N3Cc4ccccc4C3c3ccccc3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl)amino)- pyrimidin-4- yl)thiazol-4- yl)-2-fluoro- phenyl)- 1-phenylisoindoline-2- sulfonamide | 745.2 |
| 209 | 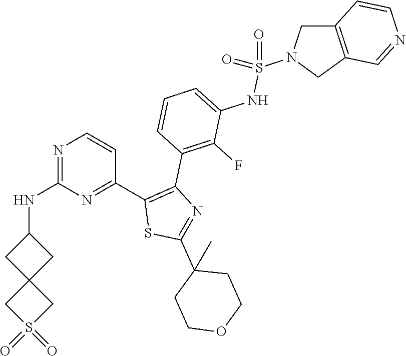 [SMILES: CC1(c2nc(-c3cccc(NS(=O)(=O)N4Cc5ccncc5C4)c3F)c(-c3ccnc(NC4CC5(C4)CS(=O)(=O)C5)n3)s2)CCOCC1] | N-(3-(5-(2-((2,2- dioxido-2-thiaspiro- [3.3]-heptan-6- yl)amino) pyrimidin-4- yl)-2-(4- methyltetrahydro- 2H-pyran-4- yl)thiazol-4-yl)-2- fluorophenyl)-1,3- dihydro-2H-pyrrolo [3,4-c]-pyridine-2- sulfonamide | 712.2 |
| 210 | 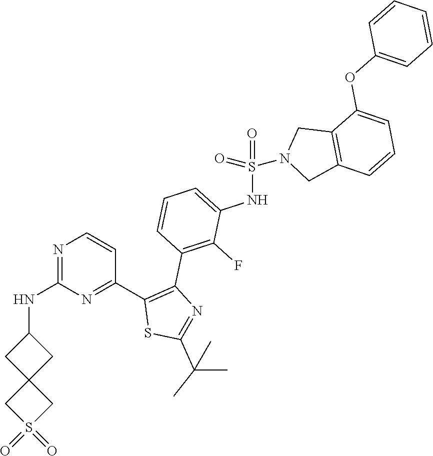 [SMILES: CC(C)(C)c1nc(-c2cccc(NS(=O)(=O)N3Cc4cccc(Oc5ccccc5)c4C3)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-4- phenoxy-isoindoline-2- sulfonamide | 761.2 |
| 211 | 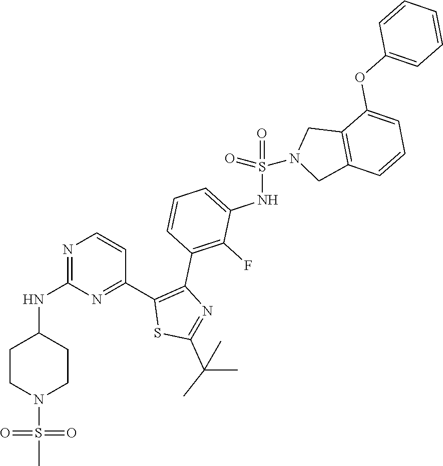 [SMILES: CC(C)(C)c1nc(-c2cccc(NS(=O)(=O)N3Cc4cccc(Oc5ccccc5)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- phenoxyisoindoline-2- sulfonamide | 778.2 |
| 212 | 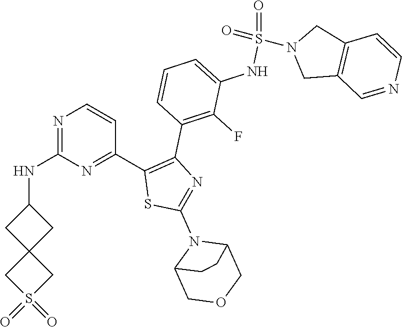 [SMILES: O=S1(=O)CC2(CC(Nc3nccc(-c4sc(N5C6CCC5COC6)nc4-c4cccc(NS(=O)(=O)N5Cc6ccncc6C5)c4F)n3)C2)C1] | N-(3-(2-(3-oxa-8- azabicyclo [3.2.1]octan- 8-yl)-5-(2-((2,2- dioxido-2-thiaspiro- [3.3]heptan-6-yl) amino)pyrimidin-4- yl)thiazol-4-yl)-2- fluorophenyl)-1,3- dihydro-2H-pyrrolo [3,4-c]-pyridine-2- sulfonamide | 725.2 |
| 213 | 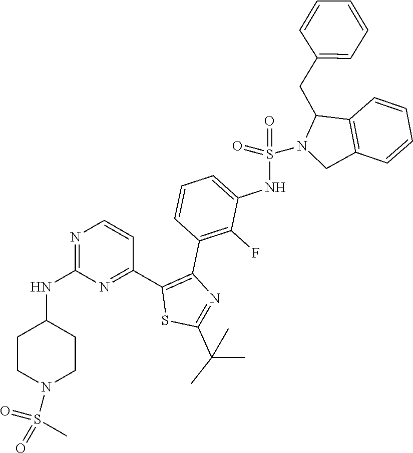 [SMILES: CC(C)(C)c1nc(-c2cccc(NS(=O)(=O)N3Cc4ccccc4C3Cc3ccccc3)c2F)c(-c2ccnc(NC3CCN(S(C)(=O)=O)CC3)n2)s1] | 1-benzyl-N-(3- (2-(tert-butyl)- 5-(2-((1- (methylsulfonyl) piperidin-4- yl)amino)- pyrimidin-4- yl)thiazol-4- yl)-2-fluoro- phenyl)- isoindoline-2- sulfonamide | 776.2 |
| 214 | 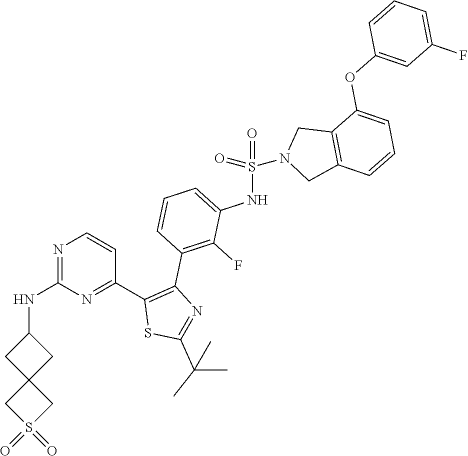 [SMILES: CC(C)(C)c1nc(-c2cccc(NS(=O)(=O)N3Cc4cccc(Oc5cccc(F)c5)c4C3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl)amino)- pyrimidin-4- yl)thiazol-4- yl)-2- fluorophenyl)-4- (3-fluorophenoxy)- isoindoline-2- sulfonamide | 779.2 |
| 215 | 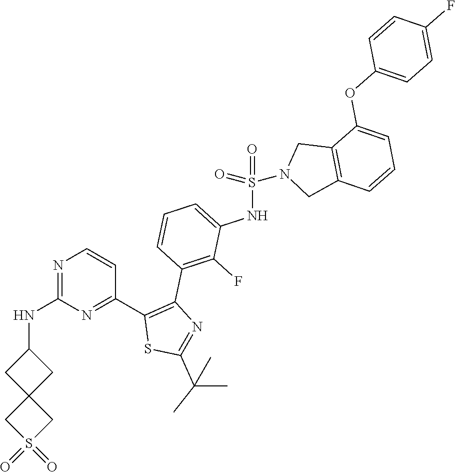 [SMILES: CC(C)(C)c1nc(-c2cccc(NS(=O)(=O)N3Cc4cccc(Oc5ccc(F)cc5)c4C3)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl)amino)- pyrimidin-4- yl)thiazol-4- yl)-2-fluoro- phenyl)-4- (4-fluoro-phenoxy)- isoindoline-2- sulfonamide | 779.2 |
| 216 | 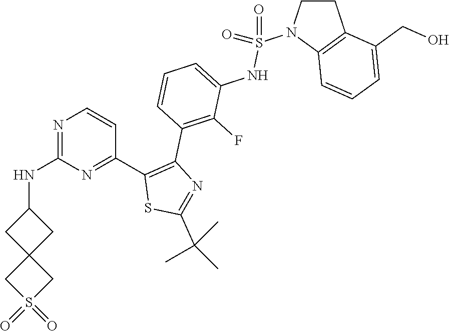 [SMILES: CC(C)(C)c1nc(-c2cccc(NS(=O)(=O)N3CCc4c(CO)cccc43)c2F)c(-c2ccnc(NC3CC4(C3)CS(=O)(=O)C4)n2)s1] | N-(3-(2- (tert-butyl)- 5-(2-((2,2- dioxido-2- thiaspiro[3.3] heptan-6-yl)amino)- pyrimidin-4- yl)thiazol-4- yl)-2-fluoro- phenyl)-4- (hydroxymethyl) indoline- 1- sulfonamide | 699.2 |
| 217 | 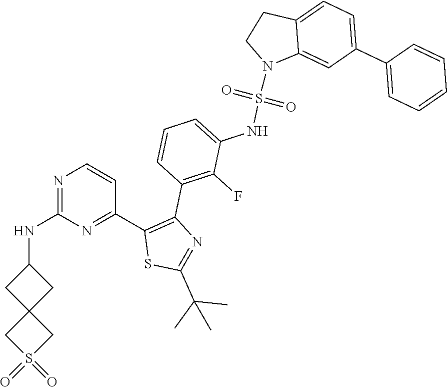 [SMILES: CC(C)(C)c1nc(-c2cccc(NS(=O)(=O)N3CCc4ccc(-c5ccccc5)cc43)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3]heptan- 6-yl)amino)- pyrimidin-4- yl)thiazol-4- yl)-2-fluoro- phenyl)-6-phenyl- indoline- 1-sulfonamide | 745.2 |
| 218 | 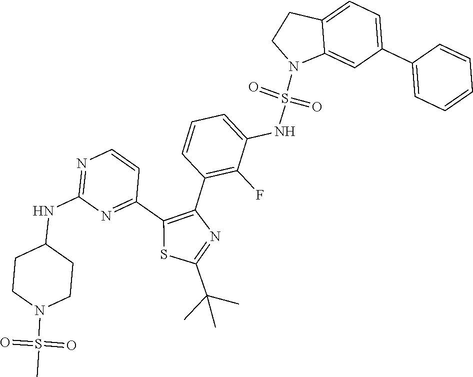 [SMILES: CC(C)(C)c1nc(-c2cccc(NS(=O)(=O)N3CCc4ccc(-c5ccccc5)cc43)c2F)c(-c2ccnc(NC3CCN(S(C)(=O)=O)CC3)n2)s1] | N-(3-(2- (tert-butyl)- 5-(2-((1- (methylsulfonyl)- piperidin-4- yl)amino)- pyrimidin-4- yl)thiazol-4- yl)-2-fluoro- phenyl)-6- phenylindoline- 1-sulfonamide | 762.2 |
| 219 | 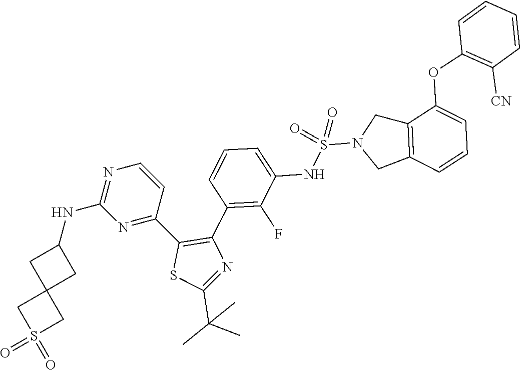 [SMILES: CC(C)(C)c1nc(-c2cccc(NS(=O)(=O)N3Cc4cccc(Oc5ccccc5C#N)c4C3)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3] heptan-6-yl)amino)- pyrimidin-4- yl)thiazol-4- yl)-2- fluorophenyl)-4- (2-cyanophenoxy)- isoindoline-2- sulfonamide | 786.1 |
| 220 | 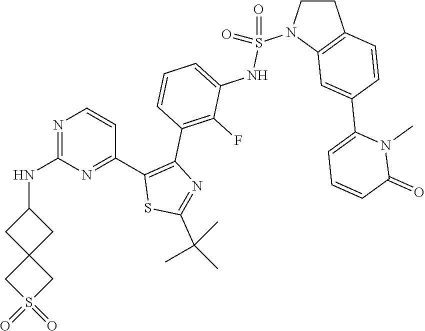 [SMILES: Cn1c(-c2ccc3c(c2)N(S(=O)(=O)Nc2cccc(-c4nc(C(C)(C)C)sc4-c4ccnc(NC5CC6(C5)CS(=O)(=O)C6)n4)c2F)CC3)cccc1=O] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-6-(1- methyl-6-oxo-1,6- dihydropyridin- 2-yl)indoline- 1- sulfonamide | 776.2 |
| 221 | 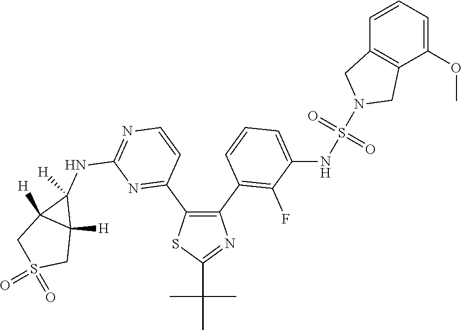 [SMILES: COc1cccc2c1CN(S(=O)(=O)Nc1cccc(-c3nc(C(C)(C)C)sc3-c3ccnc(N[C@H]4[C@@H]5CS(=O)(=O)C[C@@H]54)n3)c1F)C2] | N-(3-(2-(tert-butyl)- 5-(2-(((1R,5S,6r)- 3,3-dioxido-3- thiabicyclo [3.1.0]hexan- 6-yl)amino) pyrimidin-4- yl)thiazol-4- yl)-2- fluorophenyl)-4- methoxyisoindoline- 2-sulfonamide | 685.2 |
| 222 | 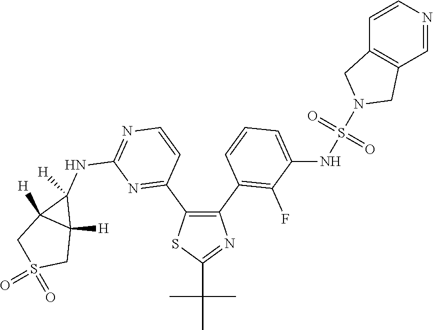 [SMILES: CC(C)(C)c1nc(-c2cccc(NS(=O)(=O)N3Cc4ccncc4C3)c2F)c(-c2ccnc(N[C@H]3[C@@H]4CS(=O)(=O)C[C@@H]43)n2)s1] | N-(3-(2-(tert-butyl)- 5-(2-(((1R,5S,6r)- 3,3-dioxido-3- thiabicyclo[3.1.0] hexan-6- yl)amino) pyrimidin-4- yl)thiazol-4- yl)-2- fluorophenyl)- 1,3-dihydro-2H- pyrrolo[3,4-c]- pyridine-2- sulfonamide | 656.2 |
| 223 | 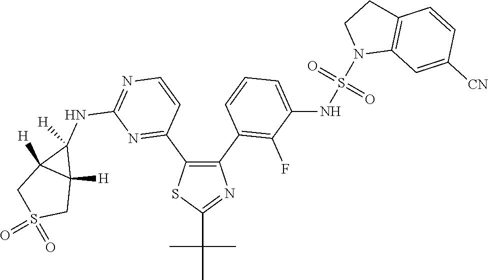 [SMILES: CC(C)(C)c1nc(-c2cccc(NS(=O)(=O)N3CCc4ccc(C#N)cc43)c2F)c(-c2ccnc(N[C@H]3[C@@H]4CS(=O)(=O)C[C@@H]43)n2)s1] | N-(3-(2-(tert-butyl)- 5-(2-(((1R,5S,6r)- 3,3-dioxido-3- thiabicyclo [3.1.0]hexan-6- yl)amino) pyrimidin-4- yl)thiazol-4- yl)-2- fluorophenyl)-6- cyanoindoline- 1-sulfonamide | 680.1 |
| 224 | 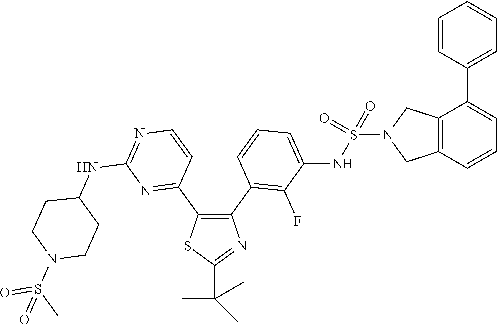 [SMILES: CC(C)(C)c1nc(-c2cccc(NS(=O)(=O)N3Cc4cccc(-c5ccccc5)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- phenylisoindoline-2- sulfonamide | 762.4 |
| 225 | 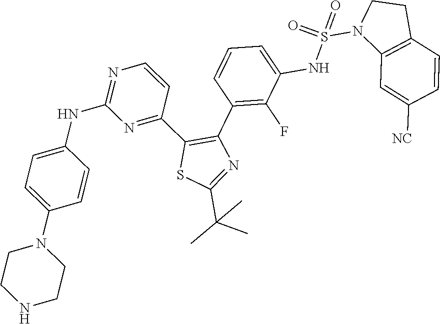 [SMILES: CC(C)(C)c1nc(-c2cccc(NS(=O)(=O)N3CCc4ccc(C#N)cc43)c2F)c(-c2ccnc(Nc3ccc(N4CCNCC4)cc3)n2)s1] | N-(3-(2-(tert-butyl)- 5-(2-((4-(piperazin- 1-yl)phenyl)amino) pyrimidin-4- yl)- thiazol-4- yl)-2-fluorophenyl)- 6-cyano- indoline- 1-sulfonamide | 710.3 |
| 226 | 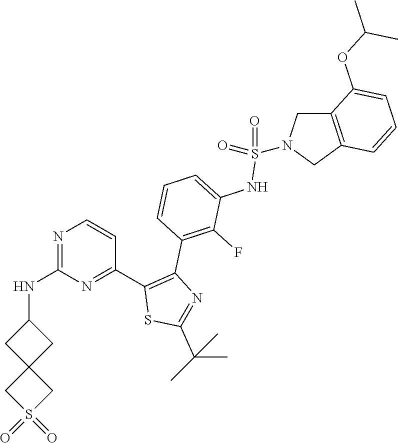 [SMILES: CC(C)Oc1cccc2c1CN(S(=O)(=O)Nc1cccc(-c3nc(C(C)(C)C)sc3-c3ccnc(NC4CC5(C4)CS(=O)(=O)C5)n3)c1F)C2] | N-(3-(2-(tert-butyl)- 5-(2-((2,2-dioxido-2- thiaspiro[3.3] heptan-6-yl)amino)- pyrimidin-4- yl)thiazol-4- yl)~2~fluoro- phenyl)-4- isopropoxyiso- indoline- 2-sulfonamide | 727.3 |
| 227 | 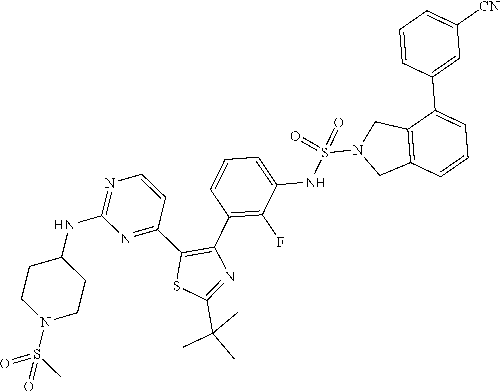 [SMILES: CC(C)(C)c1nc(-c2cccc(NS(=O)(=O)N3Cc4cccc(-c5cccc(C#N)c5)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- (3-cyanophenyl)- isoindoline- 2-sulfonamide | 787.4 |
| 228 | 2 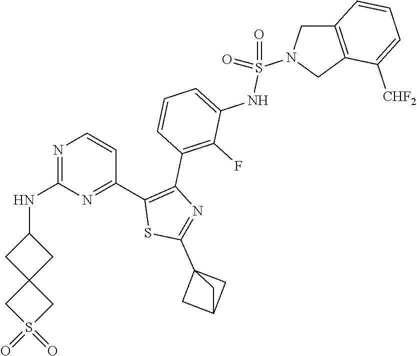 [SMILES: O=S1(=O)CC2(CC(Nc3nccc(-c4sc(C56CC(C5)C6)nc4-c4cccc(NS(=O)(=O)N5Cc6cccc(C(F)F)c6C5)c4F)n3)C2)C1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- cyclohexyliso- indoline-2- sulfonamide | 768.4 |
| 229 | 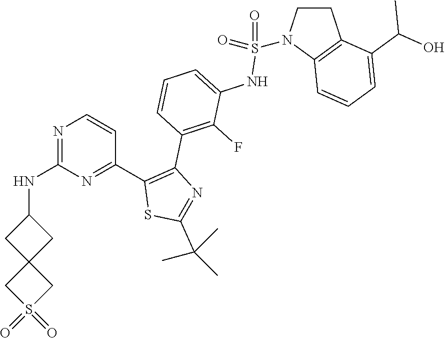 [SMILES: CC(O)c1cccc2c1CCN2S(=O)(=O)Nc1cccc(-c2nc(C(C)(C)C)sc2-c2ccnc(NC3CC4(C3)CS(=O)(=O)C4)n2)c1F] | N-(3-(2- (tert-butyl)- 5-(2-((2,2- dioxido-2- thiaspiro[3.3] heptan-6-yl) amino)- pyrimidin-4- yl)thiazol-4- yl)-2-fluoro- phenyl)-4- (1-hydroxyethyl)- indoline- 1- sulfonamide | 713.3 |
| 230 | 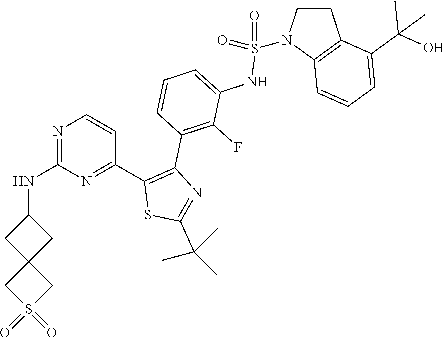 [SMILES: CC(C)(C)c1nc(-c2cccc(NS(=O)(=O)N3CCc4c3cccc4C(C)(C)O)c2F)c(-c2ccnc(NC3CC4(C3)CS(=O)(=O)C4)n2)s1] | N-(3-(2-(tert- butyl)-5-(2- ((2,2-dioxido-2- thiaspiro[3.3] heptan-6-yl)amino)- pyrimidin-4- yl)thiazol-4- yl)~2-fluoro- phenyl)-4- (2-hydroxypropan- 2-yl)-indoline- 1-sulfonamide | 726.7 |
| 231 | 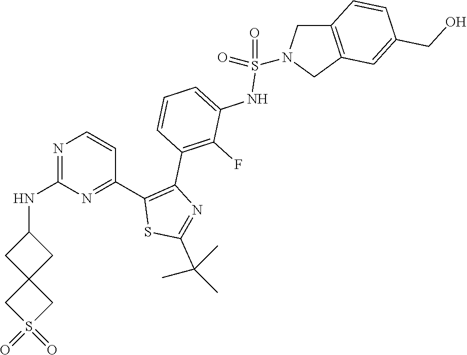 [SMILES: CC(C)(C)c1nc(-c2cccc(NS(=O)(=O)N3Cc4ccc(CO)cc4C3)c2F)c(-c2ccnc(NC3CC4(C3)CS(=O)(=O)C4)n2)s1] | N-(3-(2-(tert- butyl)- 5-(2-((2,2- dioxido-2- thiaspiro[3.3] heptan-6- yl)amino)- pyrimidin-4- yl)thiazol-4-yl)-2- fluorophenyl)-5- (hydroxymethyl)- isoindoline-2- sulfonamide | 699.3 |
| 232 | 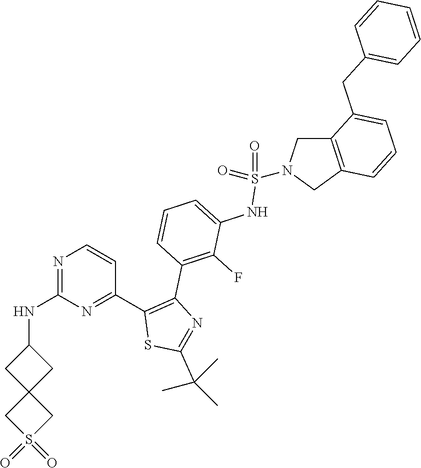 [SMILES: CC(C)(C)c1nc(-c2cccc(NS(=O)(=O)N3Cc4cccc(Cc5ccccc5)c4C3)c2F)c(-c2ccnc(NC3CC4(C3)CS(=O)(=O)C4)n2)s1] | 4-benzyl-N-(3- (2-(tert-butyl)- 5-(2-((2,2- dioxido-2- thiaspiro[3.3] heptan-6- yl)amino)- pyrimidin-4- yl)thiazol-4- yl)-2- fluorophenyl)- isoindoline-2- sulfonamide | 759.3 |
| 233 | 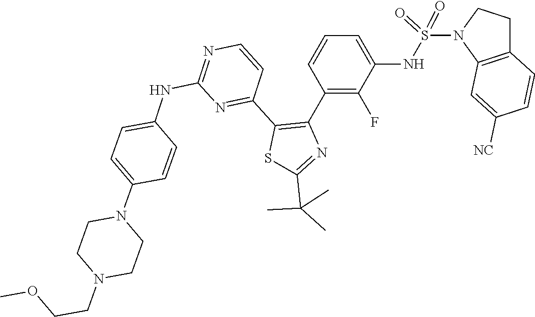 [SMILES: COCCN1CCN(c2ccc(Nc3nccc(-c4sc(C(C)(C)C)nc4-c4cccc(NS(=O)(=O)N5CCc6ccc(C#N)cc65)c4F)n3)cc2)CC1] | N-(3-(2- (tert-butyl)- 5-(2-((4-(4-(2- methoxy-ethyl) piperazin- 1-yl)phenyl)- amino)- pyrimidin-4- yl)thiazol-4- yl)-2- fluorophenyl)- 6-cyanoindoline- 1-sulfonamide | 767.7 |
| 234 | 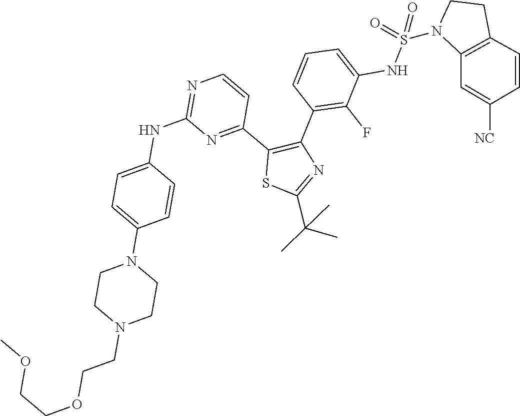 [SMILES: COCCOCCN1CCN(c2ccc(Nc3nccc(-c4sc(C(C)(C)C)nc4-c4cccc(NS(=O)(=O)N5CCc6ccc(C#N)cc65)c4F)n3)cc2)CC1] | N-(3-(2- (tert-butyl)- 5-(2-((4- (4-(2-(2- methoxy-ethoxy) ethyl)piperazin-1- yl)phenyl) amino)- pyrimidin-4- yl)thiazol-4- yl)-2-fluorophenyl)- 6-cyanoindoline- 1-sulfonamide | 812.5 |
| 235 | 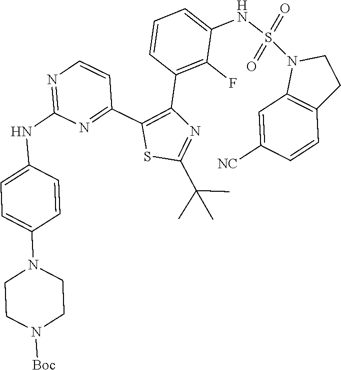 [SMILES: CC(C)(C)OC(=O)N1CCN(c2ccc(Nc3nccc(-c4sc(C(C)(C)C)nc4-c4cccc(NS(=O)(=O)N5CCc6ccc(C#N)cc65)c4F)n3)cc2)CC1] | tert-butyl 4-(4- ((4-(2-(tert-butyl)- 4-(3- ((6-cyanoindoline)- 1-sulfonamido)-2- fluorophenyl) thiazol-5-yl) pyrimidin-2- yl)amino) phenyl) piperazine-1- carboxylate | 810.3 |
| 236 | 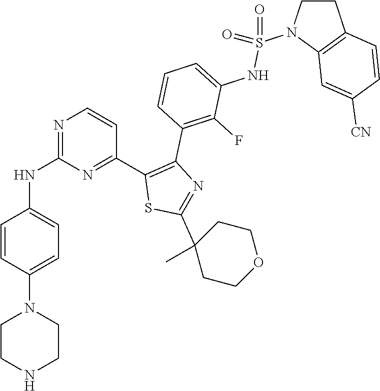 [SMILES: CC1(c2nc(-c3cccc(NS(=O)(=O)N4CCc5ccc(C#N)cc54)c3F)c(-c3ccnc(Nc4ccc(N5CCNCC5)cc4)n3)s2)CCOCC1] | 6-cyano-N-(2- fluoro-3-(2-(4- methyltetrahydro- 2H-pyran-4- yl)-5-(2- ((4-(piperazin- 1-yl)-phenyl) amino)- pyrimidin-4- yl)thiazol-4-yl)- phenyl)indoline- 1-sulfonamide | 752.3 |
| 237 | 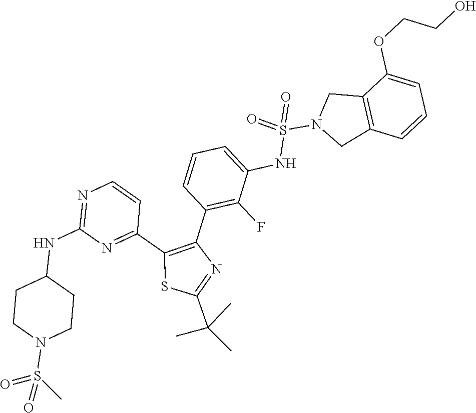 [SMILES: CC(C)(C)c1nc(-c2cccc(NS(=O)(=O)N3Cc4cccc(OCCO)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- (2-hydroxyethoxy)- isoindoline-2- sulfonamide | 746.4 |
| 238 | 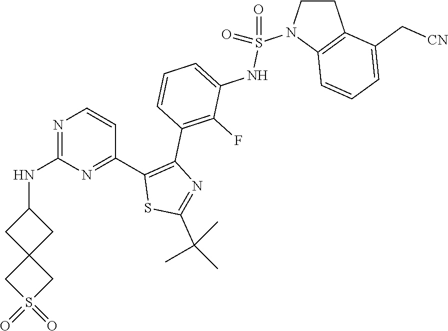 [SMILES: CC(C)(C)c1nc(-c2cccc(NS(=O)(=O)N3CCc4c(CC#N)cccc43)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-4- (cyanomethyl) indoline-1- sulfonamide | 708.2 |
| 239 | 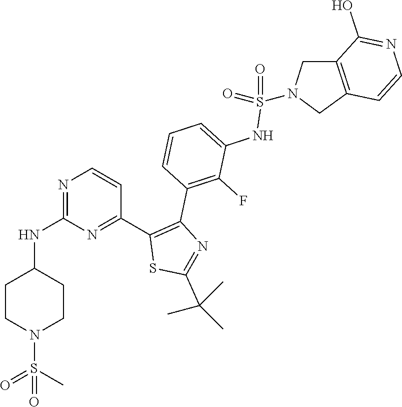 [SMILES: CC(C)(C)c1nc(-c2cccc(NS(=O)(=O)N3Cc4ccnc(O)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- hydroxy-1,3- dihydro-2H- pyrrolo[3,4-c] pyridine-2- sulfonamide | 703.2 |
| 240 | 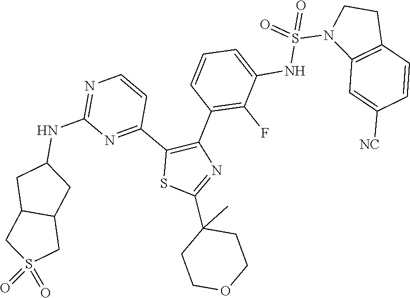 [SMILES: CC1(c2nc(-c3cccc(NS(=O)(=O)N4CCc5ccc(C#N)cc54)c3F)c(-c3ccnc(NC4CC5CS(=O)(=O)CC5C4)n3)s2)CCOCC1] | 6-cyano-N-(3-(5- (2-((2,2- dioxidohexa- hydro-1H- cyclopenta[c] thiophen-5- yl)amino)- pyrimidin-4- yl)-2-(4- methyl- tetrahydro- 2H-pyran-4- yl)thiazol-4- yl)- 2-fluorophenyl)- indoline- 1-sulfonamide | 750.2 |
| 241 | 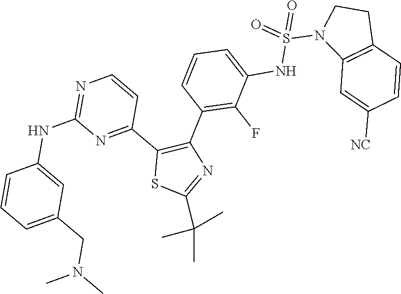 [SMILES: CN(C)Cc1cccc(Nc2nccc(-c3sc(C(C)(C)C)nc3-c3cccc(NS(=O)(=O)N4CCc5ccc(C#N)cc54)c3F)n2)c1] | N-(3-(2-(tert-butyl)- 5-(2-((3- ((dimethylamino)- methyl)phenyl)- amino)pyrimidin-4- yl)thiazol-4- yl)-2- fluorophenyl)- 6-cyanoindoline- 1-sulfonamide | 683.3 |
| 242 | 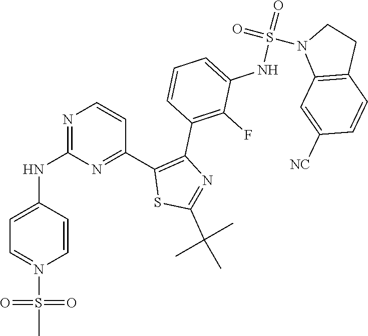 | N-(3-(2-(tert-butyl)- 5-(2-((4- (methylsulfonyl)- phenyl)amino)- pyrimidin-4- yl)thiazol-4- yl)-2-fluoro- phenyl)-6- cyanoindoline- 1-sulfonamide | 704.2 |
| 243 | 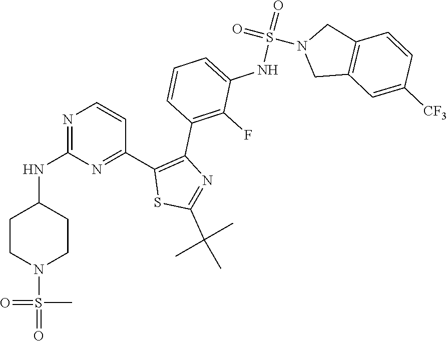 [SMILES: CC(C)(C)c1nc(-c2cccc(NS(=O)(=O)N3Cc4ccc(C(F)(F)F)cc4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-5- (trifluoromethyl)- isoindoline- 2-sulfonamide | 754.2 |
| 244 | 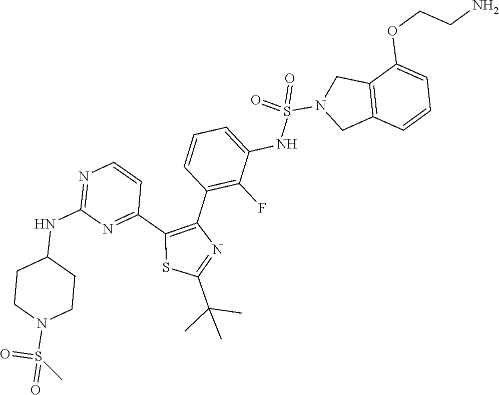 [SMILES: CC(C)(C)c1nc(-c2cccc(NS(=O)(=O)N3Cc4cccc(OCCN)c4C3)c2F)c(-c2ccnc(NC3CCN(S(C)(=O)=O)CC3)n2)s1] | 4-(2-aminoethoxy)- N-(3-(2-(tert-butyl)- 5-(2-((1- (methylsulfonyl) piperidin-4- yl)amino)- pyrimidin-4- yl)thiazol-4- yl)-2- fluorophenyl)- isoindoline-2- sulfonamide | 745.4 |
| 245 | 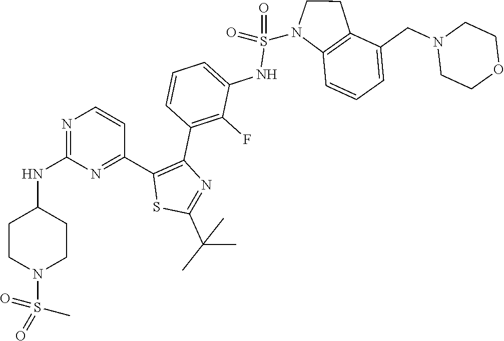 [SMILES: CC(C)(C)c1nc(-c2cccc(NS(=O)(=O)N3CCc4c(CN5CCOCC5)cccc43)c2F)c(-c2ccnc(NC3CCN(S(C)(=O)=O)CC3)n2)s1] | N-(3-(2-(tert- butyl)- 5-(2-((1- (methylsulfonyl) piperidin-4- yl)amino)- pyrimidin-4- yl)thiazol-4- yl)-2- fluorophenyl)-4- (morpholinomethyl)- indoline- 1-sulfonamide | 785.4 |
| 246 | 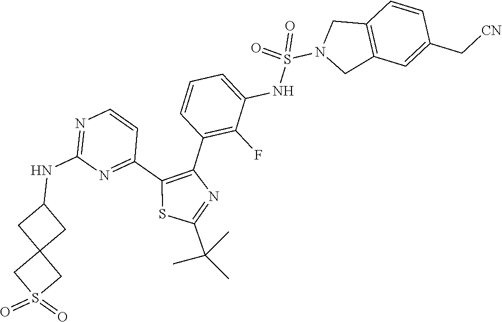 [SMILES: CC(C)(C)c1nc(-c2cccc(NS(=O)(=O)N3Cc4ccc(CC#N)cc4C3)c2F)c(-c2ccnc(NC3CC4(C3)CS(=O)(=O)C4)n2)s1] | N-(3-(2-(tert- butyl)- 5-(2-((2,2- dioxido-2- thiaspiro[3.3] heptan-6-yl) amino)- pyrimidin-4- yl)thiazol-4- yl)-2-fluoro- phenyl)-5- (cyanomethyl)- isoindoline-2- sulfonamide | 708.2 |
| 247 | 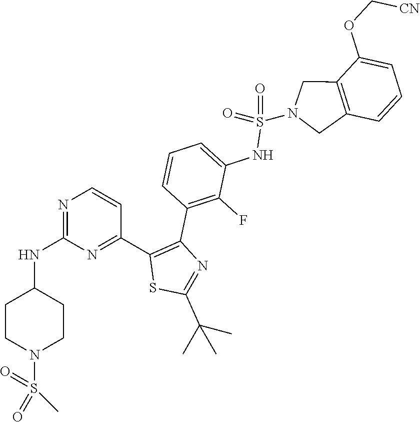 [SMILES: CC(C)(C)c1nc(-c2cccc(NS(=O)(=O)N3Cc4cccc(OCC#N)c4C3)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)-4- (cyanomethoxy)- isoindoline- 2-sulfonamide | 741.2 |
| 248 | 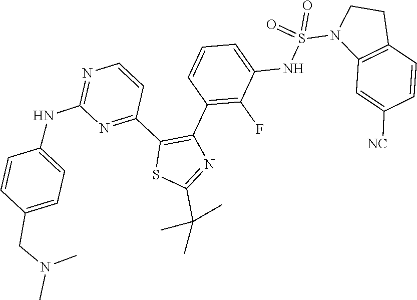 [SMILES: CN(C)Cc1ccc(Nc2nccc(-c3sc(C(C)(C)C)nc3-c3cccc(NS(=O)(=O)N4CCc5ccc(C#N)cc54)c3F)n2)cc1] | N-(3-(2-(tert- butyl)~5-(2-((4- ((dimethylamino)- methyl)phenyl)- amino)pyrimidin-4- yl)thiazol-4- yl)-2- fluorophenyl)-6- cyanoindoline- 1- sulfonamide | 683.3 |
| 249 | 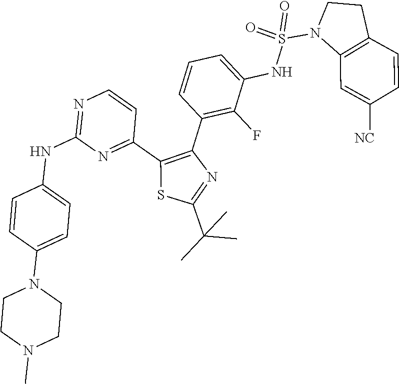 [SMILES: CN1CCN(c2ccc(Nc3nccc(-c4sc(C(C)(C)C)nc4-c4cccc(NS(=O)(=O)N5CCc6ccc(C#N)cc65)c4F)n3)cc2)CC1] | N-(3-(2-(tert-butyl)- 5-(2-((4-(4- methyl- piperazin- 1-yl)phenyl) amino)pyrimidin- 4- yl)-thiazol-4- yl)-2-fluoro- phenyl)-6- cyanoindoline- 1-sulfonamide | 724.3 |
| 250 | 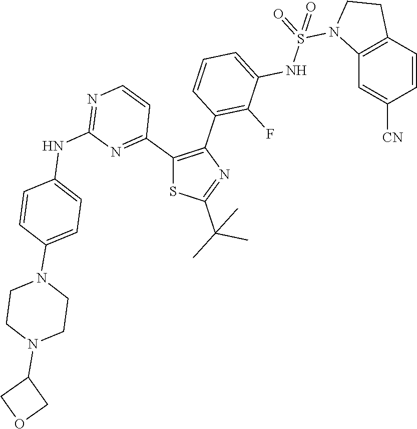 [SMILES: CC(C)(C)c1nc(-c2cccc(NS(=O)(=O)N3CCc4ccc(C#N)cc43)c2F)c(-c2ccnc(Nc3ccc(N4CCN(C5COC5)CC4)cc3)n2)s1] | N-(3-(2-(tert-butyl)- 5-(2-((4-(4-(oxetan- 3-yl)- piperazin- 1-yl)phenyl) amino)- pyrimidin-4- yl)thiazol-4- yl)-2-fluoro- phenyl)-6- cyanoindoline- 1-sulfonamide | 766.3 |
| 251 | 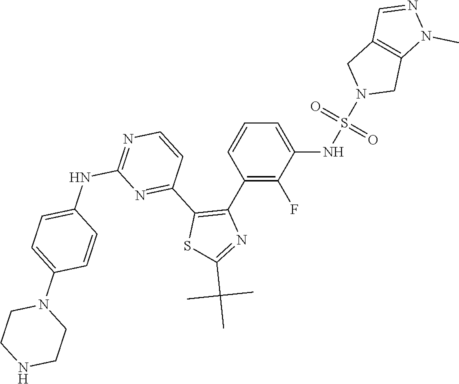 [SMILES: Cn1ncc2c1CN(S(=O)(=O)Nc1cccc(-c3nc(C(C)(C)C)sc3-c3ccnc(Nc4ccc(N5CCNCC5)cc4)n3)c1F)C2] | N-(3-(2-(tert-butyl)- 5-(2-((4-(piperazin- 1-yl)phenyl)amino) pyrimidin-4- yl)-thiazol-4- yl)-2- fluorophenyl)- 1-methyl- 4,6-dihydropyrrolo- [3,4-c]pyrazole- 5(1H)-sulfonamide | 689.3 |
| 252 | 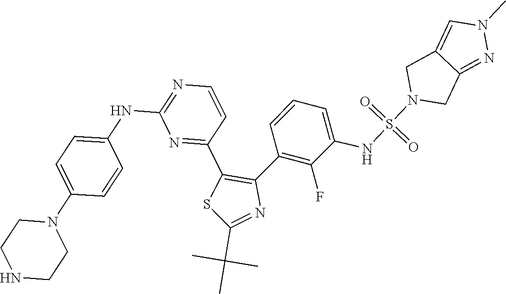 [SMILES: Cn1cc2c(n1)CN(S(=O)(=O)Nc1cccc(-c3nc(C(C)(C)C)sc3-c3ccnc(Nc4ccc(N5CCNCC5)cc4)n3)c1F)C2] | N-(3-(2-(tert-butyl)- 5-(2-((4-(piperazin- 1-yl)-phenyl)amino) pyrimidin-4- yl)-thiazol-4- yl)-2-fluorophenyl)-2- methyl- 2,6-dihydropyrrolo- [3,4-c]pyrazole- 5(4H)-sulfonamide | 689.2 |
| 253 | 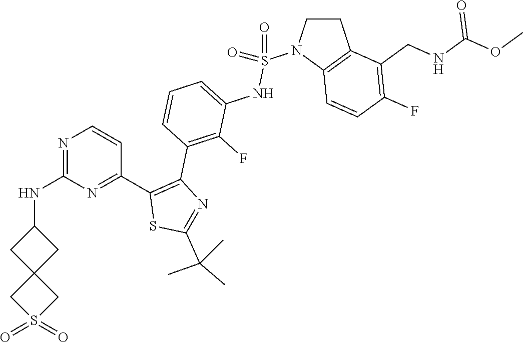 [SMILES: COC(=O)NCc1c(F)ccc2c1CCN2S(=O)(=O)Nc1cccc(-c2nc(C(C)(C)C)sc2-c2ccnc(NC3CC4(C3)CS(=O)(=O)C4)n2)c1F] | methyl ((1-(N-(3- (2-(tert-butyl)- S-(2- ((2,2-dioxido-2- thiaspiro[3.3]heptan-6- yl)amino)-pyrimidin-4- yl)thiazol-4- yl)-2- fluorophenyl)- sulfamoyl)indolin-4- yl)methyl)carbamate | 756.3 |
| 254 | 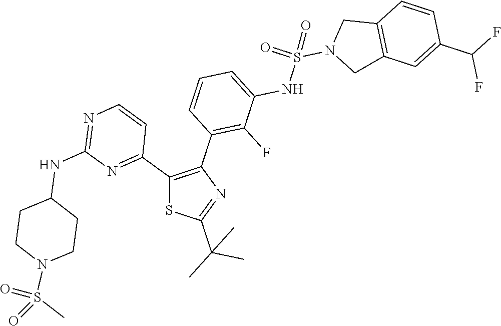 [SMILES: CC(C)(C)c1nc(-c2cccc(NS(=O)(=O)N3Cc4ccc(C(F)F)cc4C3)c2F)c(-c2ccnc(NC3CCN(S(C)(=O)=O)CC3)n2)s1] | N-(3-(2-(tert-butyl)- 5-(2-((1+ (methylsulfonyl)- piperidin-4- yl)amino)- pyrimidin-4- yl)thiazol-4- yl)-2-fluoro- phenyl)-5- (difluoromethyl)- isoindoline- 2-sulfonamide | 736.3 |
| 255 | 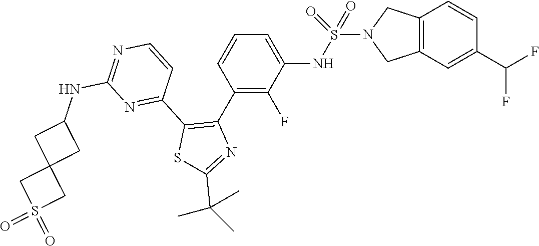 [SMILES: CC(C)(C)c1nc(-c2cccc(NS(=O)(=O)N3Cc4ccc(C(F)F)cc4C3)c2F)c(-c2ccnc(NC3CC4(C3)CS(=O)(=O)C4)n2)s1] | N-(3-(2-(tert- butyl)-5-(2-((2,2- dioxido-2- thiaspiro[3.3] heptan-6-yl)amino)- pyrimidin-4- yl)thiazol-4- yl)-2-fluoro- phenyl)-5- (difluoromethyl) isoindoline-2- sulfonamide | 719.3 |
| 256 | 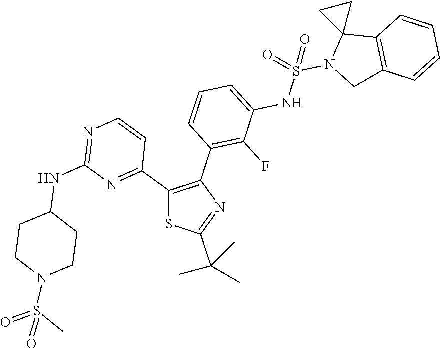 [SMILES: CC(C)(C)c1nc(-c2cccc(NS(=O)(=O)N3Cc4ccccc4C34CC4)c2F)c(-c2ccnc(NC3CCN(S(C)(=O)=O)CC3)n2)s1] | N-(3-(2-(tert-butyl)- 5-(2-((1- (methylsulfonyl)- piperidin-4- yl)amino)- pyrimidin-4- yl)thiazol-4- yl)-2-fluoro- phenyl)spiro [cyclopropane-1,1′- isoindoline]-2′- sulfonamide | 712.3 |
| 257 | 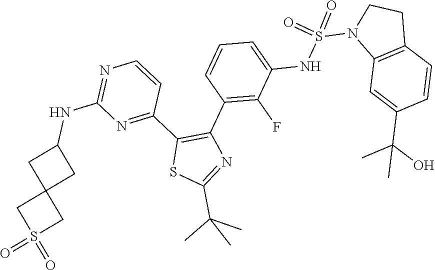 [SMILES: CC(C)(C)c1nc(-c2cccc(NS(=O)(=O)N3CCc4ccc(C(C)(C)O)cc43)c2F)c(-c2ccnc(NC3CC4(C3)CS(=O)(=O)C4)n2)s1] | N-(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-6-(2- hydroxypropan-2- yl)indoline- 1-sulfonamide | 727.2 |
| 258 | 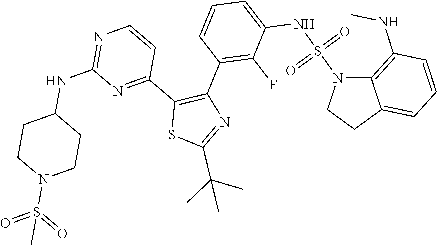 [SMILES: CNc1cccc2c1N(S(=O)(=O)Nc1cccc(-c3nc(C(C)(C)C)sc3-c3ccnc(NC4CCN(S(C)(=O)=O)CC4)n3)c1F)CC2] | N-(3-(2-(tert-butyl)- 5-(2-((1- (methylsulfonyl)- piperidin-4- yl)amino)- pyrimidin-4- yl)thiazol-4- yl)-2-fluoro- phenyl)-7- (methylamino) indoline- 1-sulfonamide | 715.4 |
| 259 | 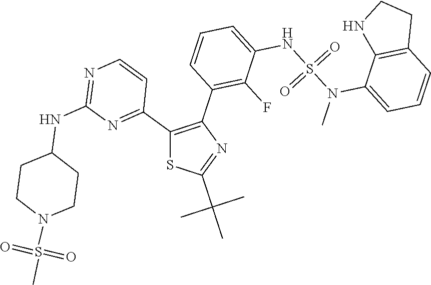 [SMILES: CN(c1cccc2c1NCC2)S(=O)(=O)Nc1cccc(-c2nc(C(C)(C)C)sc2-c2ccnc(NC3CCN(S(C)(=O)=O)CC3)n2)c1F] | N-(3-(2-(tert-butyl)- 5-(2-((1- (methylsulfonyl)- piperidin-4- yl)amino)- pyrimidin-4- yl)thiazol-4- yl)-2-fluoro- phenyl)-(N- methyl-N- indolin-7-yl)- amine-sulfonamide | 715.4 |
| 260 | 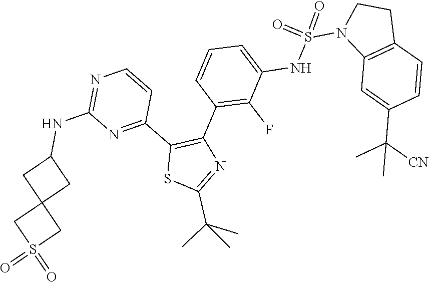 [SMILES: CC(C)(C)c1nc(-c2cccc(NS(=O)(=O)N3CCc4ccc(C(C)(C)C#N)cc43)c2F)c(-c2ccnc(NC3CC4(C3)CS(=O)(=O)C4)n2)s1] | N-(3-(2- (tert-butyl)- S-(2-((2,2- dioxido-2- thiaspiro[3.3] heptan-6-yl)amino)- pyrimidin-4- yl)thiazol-4- yl)-2-fluoro- phenyl)-6-(2- cyanopropan- 2-yl)indoline- 1-sulfonamide | 736.3 |
| 261 | 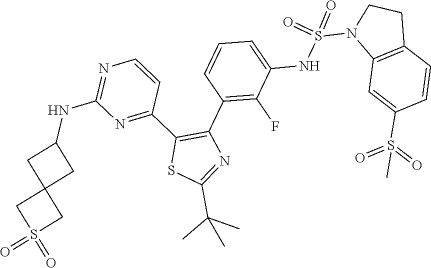 [SMILES: CC(C)(C)c1nc(-c2cccc(NS(=O)(=O)N3CCc4ccc(S(C)(=O)=O)cc43)c2F)c(-c2ccnc(NC3CC4(C3)CS(=O)(=O)C4)n2)s1] | N-(3-(2-(tert- butyl)-5-(2-((2, 2-dioxido-2- thiaspiro[3.3] heptan-6-yl)amino)- pyrimidin-4- yl)thiazol-4- yl)-2-fluoro- phenyl)-6- (methylsulfonyl)- indoline-1- sulfonamide | 747.2 |
| 262 | 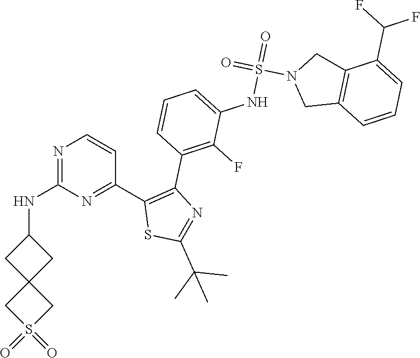 [SMILES: CC(C)(C)c1nc(-c2cccc(NS(=O)(=O)N3Cc4cccc(C(F)F)c4C3)c2F)c(-c2ccnc(NC3CC4(C3)CS(=O)(=O)C4)n2)s1] | N~(3-(2-(tert-butyl)- 5-(2-((2,2-dioxido-2- thiaspiro[3.3] heptan-6-yl)amino)- pyrimidin-4- yl)thiazol-4- yl)-2-fluoro- phenyl)-4- (difluoromethyl)- isoindoline- 2-sulfonamide | 719.2 |
| 263 | 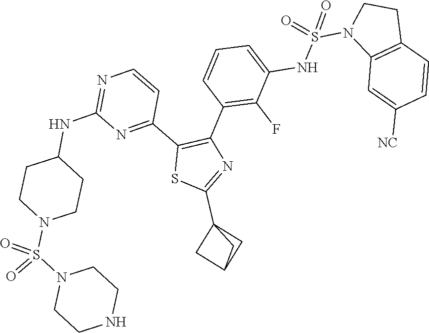 [SMILES: N#Cc1ccc2c(c1)N(S(=O)(=O)Nc1cccc(-c3nc(C45CC(C4)C5)sc3-c3ccnc(NC4CCN(S(=O)(=O)N5CCNCC5)CC4)n3)c1F)CC2] | N-(3-(2-(bicyclo [1.1.1]pentan- 1-yl)-5-(2- ((1-(piperazin- 1-ylsulfonyl) piperidin-4- yl)amino)- pyrimidin-4- yl)thiazol-4- yl)-2- fluorophenyl)- 6-cyanoindoline- 1-sulfonamide | 791.2 |
| 264 | 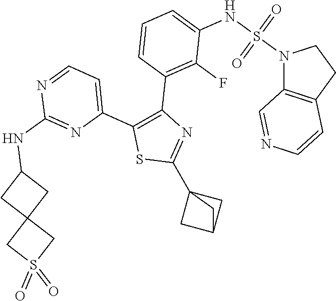 [SMILES: O=S1(=O)CC2(CC(Nc3nccc(-c4sc(C56CC(C5)C6)nc4-c4cccc(NS(=O)(=O)N5CCc6ccncc65)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 2,3-dihydro- 1H- pyrrolo[2,3- c]pyridine- 1-sulfonamide | 680.2 |
| 265 | 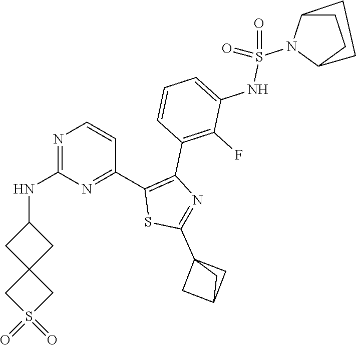 [SMILES: O=S1(=O)CC2(CC(Nc3nccc(-c4sc(C56CC(C5)C6)nc4-c4cccc(NS(=O)(=O)N5C6CCC5CC6)c4F)n3)C2)C1] | N-(3-(2- (bicyclo[1.1. 1]pentan- 1-yl)-5-(2- ((2,2-dioxido-2- thiaspiro[3.3] heptan-6- yl)amino)- pyrimidin-4- yl)thiazol-4-yl)-2- fluorophenyl)-7- azabicyclo[2.2.1]- heptane-7- sulfonamide | 657.2 |
| 266 | 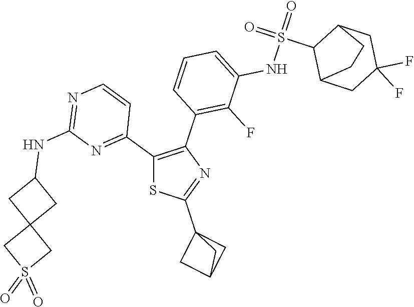 [SMILES: O=S1(=O)CC2(CC(Nc3nccc(-c4sc(C56CC(C5)C6)nc4-c4cccc(NS(=O)(=O)C5C6CCC5CC(F)(F)C6)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)-3,3- difluoro-8- azabicyclo- [3.2.1]octane-8- sulfonamide | 707.2 |
| 267 | 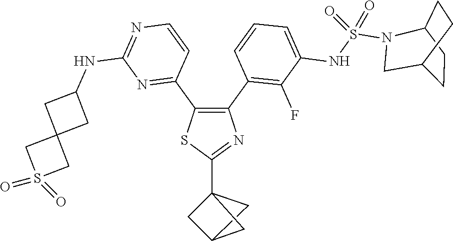 [SMILES: O=S1(=O)CC2(CC(Nc3nccc(-c4sc(C56CC(C5)C6)nc4-c4cccc(NS(=O)(=O)N5CC6CCC5CC6)c4F)n3)C2)C1] | (1s,4s)-N-(3-(2- (bicyclo[1.1.1] pentan-]- yl)-5-(2-((2,2- dioxido-2-thiaspiro- [3.3]heptan-6- yl)amino)pyrimidin- 4-yl)thiazol-4- yl)-2- fluorophenyl)-2- azabicyclo[2.2.2] octane-2-sulfonamide | 671.2 |
| 268 | 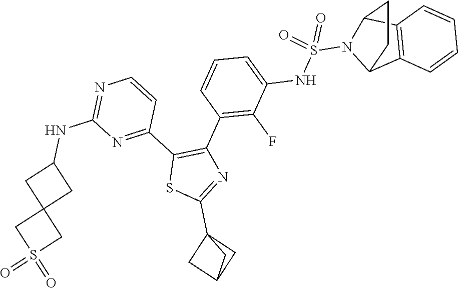 [SMILES: O=S1(=O)CC2(CC(Nc3nccc(-c4sc(C56CC(C5)C6)nc4-c4cccc(NS(=O)(=O)N5C6CCC5c5ccccc56)c4F)n3)C2)C1] | N-(3-(2-(bicyclo [1.1.1]pentan- 1-yl)-5-(2- ((2,2-dioxido-2- thiaspiro[3.3] heptan-6- yl)amino)- pyrimidin-4- yl)thiazol-4- yl)-2- fluorophenyl)- 1,2,3,4-tetrahydro- 1,4- epiminonaphthalene- 9-sulfonamide | 705.2 |
| 269 | 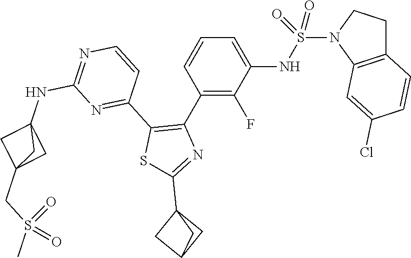 [SMILES: CS(=O)(=O)CC12CC(Nc3nccc(-c4sc(C56CC(C5)C6)nc4-c4cccc(NS(=O)(=O)N5CCc6ccc(Cl)cc65)c4F)n3)(C1)C2] | N-(3-(2-(bicyclo [1.1.1]pentan- 1-yl)-5-(2- ((3-((methylsulfonyl) methyl)bicyclo- [1.1.1 ]pentan- 1-yl)amino) pyrimidin-4- yl)thiazol-4- yl)-2-fluorophenyl)-6- chloroindoline- 1-sulfonamide | 727.3 |
| 270 | 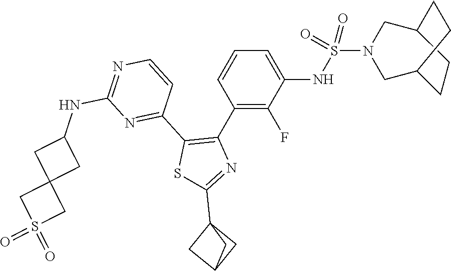 [SMILES: O=S1(=O)CC2(CC(Nc3nccc(-c4sc(C56CC(C5)C6)nc4-c4cccc(NS(=O)(=O)N5CC6CCC(CC6)C5)c4F)n3)C2)C1] | (1s,5s)-N-(3-(2- (bicyclo[1.1.1] pentan-1- yl)-5-(2-((2,2- dioxido-2- thiaspiro[3.3]- heptan-6-yl)- amino)pyrimidin-4- yl)thiazol-4- yl)-2-fluorophenyl)-3- azabicyclo[3.2.2] nonane-3-sulfonamide | 685.2 |
| 271 | 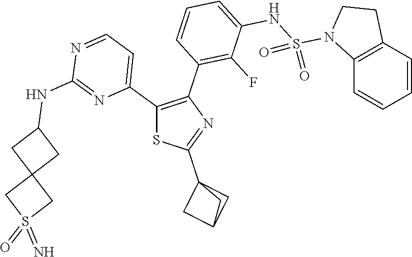 [SMILES: N=S1(=O)CC2(CC(Nc3nccc(-c4sc(C56CC(C5)C6)nc4-c4cccc(NS(=O)(=O)N5CCc6ccccc65)c4F)n3)C2)C1] | N-(3-(2-(bicyclo [1.1.1]pentan- 1-yl)-5-(2- ((2-imino-2-oxido- 26-thiaspiro[3.3]- heptan-6-yl)- amino)pyrimidin-4- yl)thiazol-4- yl)-2-fluorophenyl) indoline- 1-sulfonamide | 678.3 |
| 272 | 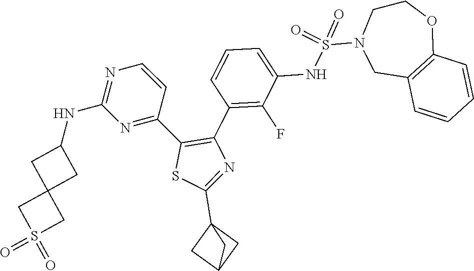 [SMILES: O=S1(=O)CC2(CC(Nc3nccc(-c4sc(C56CC(C5)C6)nc4-c4cccc(NS(=O)(=O)N5CCOc6ccccc6C5)c4F)n3)C2)C1] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-2,3- dihydrobenzo- [f][1,4]oxazepine- 4(5H)-sulfonamide | 709.3 |
| 273 | 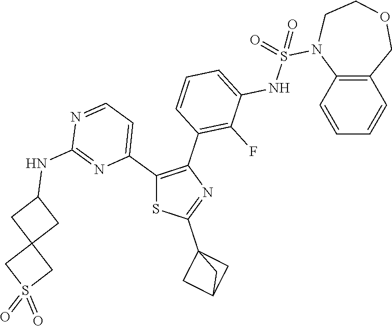 [SMILES: O=S1(=O)CC2(CC(Nc3nccc(-c4sc(C56CC(C5)C6)nc4-c4cccc(NS(=O)(=O)N5CCOCc6ccccc65)c4F)n3)C2)C1] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4-yl)-2- fluorophenyl)-2,3- dihydrobenzo- [e][ 1,4]oxazepine- 1(5H)-sulfonamide | 709.2 |
| 274 | 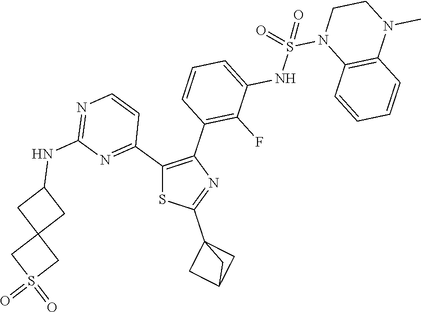 [SMILES: CN1CCN(S(=O)(=O)Nc2cccc(-c3nc(C45CC(C4)C5)sc3-c3ccnc(NC4CC5(C4)CS(=O)(=O)C5)n3)c2F)c2ccccc21] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-4- methyl-3,4-dihydro- quinoxaline-1(2H)- sulfonamide | 708.2 |
| 275 | 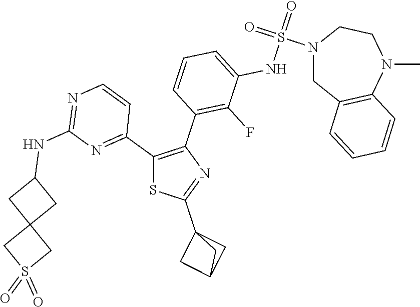 [SMILES: CN1CCN(S(=O)(=O)Nc2cccc(-c3nc(C45CC(C4)C5)sc3-c3ccnc(NC4CC5(C4)CS(=O)(=O)C5)n3)c2F)Cc2ccccc21] | N-(3-(2-(bicyclo [1.1.1]pentan- 1-yl)-5-(2- ((2,2-dioxido-2- thiaspiro[3.3]heptan-6- yl)amino)-pyrimidin-4- yl)thiazol-4- yl)-2- fluorophenyl)-1- methyl-1,2,3,5- tetrahydro-4H- benzo[e][1,4]- diazepine- 4-sulfonamide | 722.4 |
| 276 | 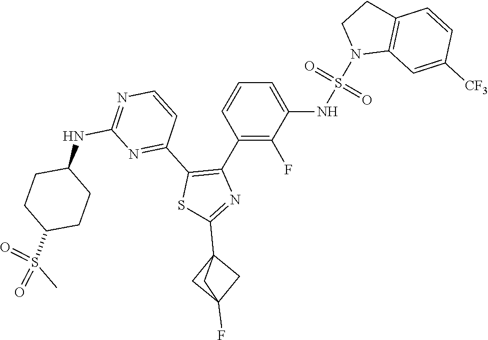 [SMILES: CS(=O)(=O)[C@H]1CC[C@H](Nc2nccc(-c3sc(C45CC(F)(C4)C5)nc3-c3cccc(NS(=O)(=O)N4CCc5ccc(C(F)(F)F)cc54)c3F)n2)CC1] | N-(2-fluoro-3- (2-(3-fluorobicyclo- [1.1.1 ]pentan- 1-yl)-5-(2- (((1s,4s)-4- (methylsulfonyl)- cyclohexyl)amino)- pyrimidin-4- yl)thiazol-4- yl)phenyl)-6- (trifluoromethyl) indoline- 1-sulfonamide | 781.2 |
| 277 | 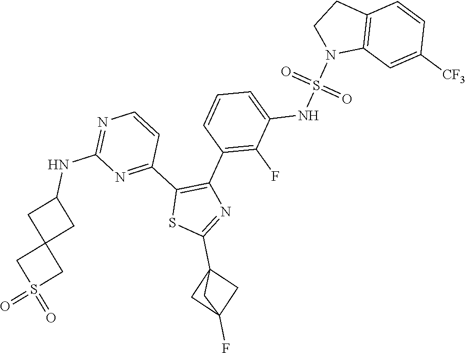 [SMILES: O=S1(=O)CC2(CC(Nc3nccc(-c4sc(C56CC(F)(C5)C6)nc4-c4cccc(NS(=O)(=O)N5CCc6ccc(C(F)(F)F)cc65)c4F)n3)C2)C1] | N-(3-(5-(2-((2,2- dioxido-2-thiaspiro- [3.3]-heptan-6-yl) amino)pyrimidin-4- yl)- 2-(3-fluorobicyclo [1.1.1]pentan-1-yl)- thiazol-4-yl)-2- fluorophenyl)-6- (trifluoromethyl) indoline- 1-sulfonamide | 765.1 |
| 278 | 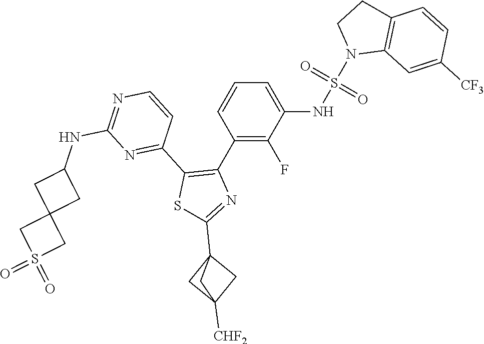 [SMILES: O=S1(=O)CC2(CC(Nc3nccc(-c4sc(C56CC(C(F)F)(C5)C6)nc4-c4cccc(NS(=O)(=O)N5CCc6ccc(C(F)(F)F)cc65)c4F)n3)C2)C1] | N-(3-(2-(3- (difluoromethyl) bicyclo- [1.1.1]-pentan- 1-yl)-5-(2-((2, 2-dioxido- 2-thiaspiro[3.3] heptan-6-yl)amino)- pyrimidin-4- yl)thiazol-4- yl)-2- fluorophenyl)-6- (trifluoromethyl)- indoline- 1-sulfonamide | 797.1 |
| 279 | 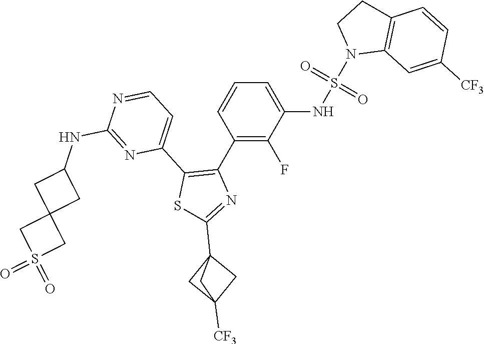 [SMILES: O=S1(=O)CC2(CC(Nc3nccc(-c4sc(C56CC(C(F)(F)F)(C5)C6)nc4-c4cccc(NS(=O)(=O)N5CCc6ccc(C(F)(F)F)cc65)c4F)n3)C2)C1] | N-(3~(5-(2-((2,2- dioxido-2-thiaspiro- [3.3]heptan-6-yl) amino)pyrimidin-4- yl)-2-(3- (trifluoromethyl) bicyclo[1.1.1]- pentan- 1-yl)thiazol-4- yl)-2-fluoro- phenyl)-6- (trifluoromethyl) indoline- 1-sulfonamide | 815.0 |
| 280 | 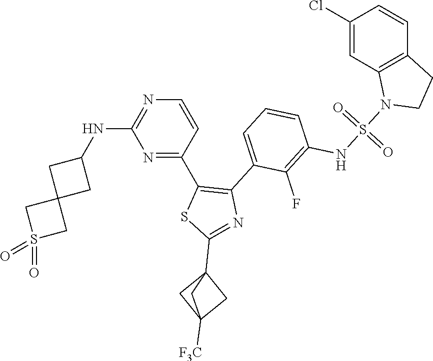 [SMILES: O=S1(=O)CC2(CC(Nc3nccc(-c4sc(C56CC(C(F)(F)F)(C5)C6)nc4-c4cccc(NS(=O)(=O)N5CCc6ccc(Cl)cc65)c4F)n3)C2)C1] | 6-chloro-N-(3-(5- (2-((2,2-dioxido-2- thiaspiro[3.3] heptan-6-yl)amino)- pyrimidin-4- yl)-2-(3- (trifluoromethyl)- bicyclo[1.1.1] pentan- 1-yl)thiazol-4-yl)- 2-fluorophenyl) indoline- 1-sulfonamide | 780.9 |
| 281 | 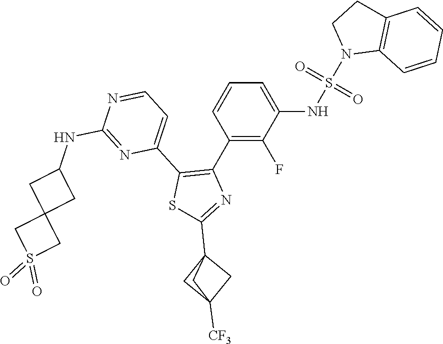 [SMILES: O=S1(=O)CC2(CC(Nc3nccc(-c4sc(C56CC(C(F)(F)F)(C5)C6)nc4-c4cccc(NS(=O)(=O)N5CCc6ccccc65)c4F)n3)C2)C1] | N-(3-(5-(2-((2,2- dioxido-2-thiaspiro- [3.3]heptan-6-y]) amino)pyrimidin-4- yl)- 2-(3-(trifluoromethyl) bicyclo[1.1.1]- pentan- 1-yl)thiazol-4- yl)-2-fluoro- phenyl)indoline- 1-sulfonamide | 747.10 |
| 282 | 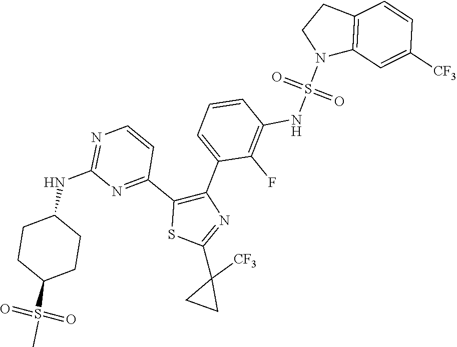 [SMILES: CS(=O)(=O)[C@H]1CC[C@H](Nc2nccc(-c3sc(C4(C(F)(F)F)CC4)nc3-c3cccc(NS(=O)(=O)N4CCc5ccc(C(F)(F)F)cc54)c3F)n2)CC1] | N-(2-fluoro-3-(5- (2-(((1r,4r)-4- (methylsulfonyl) cyclohexyl)amino) pyrimidin-4- yl)-2-(1- (trifluoromethyl)- cyclopropyl) thiazol-4- yl)phenyl)-6- (trifluoromethyl) indoline- 1-sulfonamide | 804.9 |
| 283 | 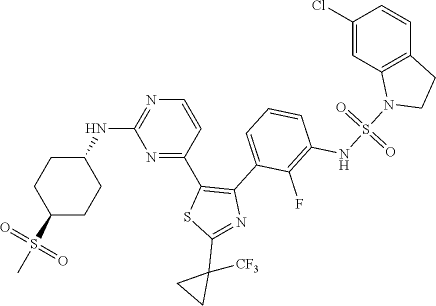 [SMILES: CS(=O)(=O)[C@H]1CC[C@H](Nc2nccc(-c3sc(C4(C(F)(F)F)CC4)nc3-c3cccc(NS(=O)(=O)N4CCc5ccc(Cl)cc54)c3F)n2)CC1] | 6-chloro-N-(2-fluoro- 3-(5-(2-(((1r,4r)-4- (methylsulfonyl) cyclohexyl)amino) pyrimidin-4- yl)-2-(1- (trifluoromethyl)- cyclopropyl)thiazol-4- yl)phenyl)- indoline- 1-sulfonamide | 770.9 |
| 284 | 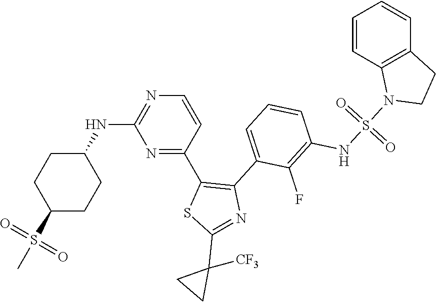 [SMILES: CS(=O)(=O)[C@H]1CC[C@H](Nc2nccc(-c3sc(C4(C(F)(F)F)CC4)nc3-c3cccc(NS(=O)(=O)N4CCc5ccccc54)c3F)n2)CC1] | N-(2-fluoro-3-(5- (2-(((11,4r)-4- (methylsulfonyl) cyclohexyl)amino) pyrimidin-4- yl)-2-(1- (trifluoromethyl)- cyclopropyl) thiazol-4- yl)phenyl)- indoline- 1-sulfonamide | 737.0 |
| 285 | 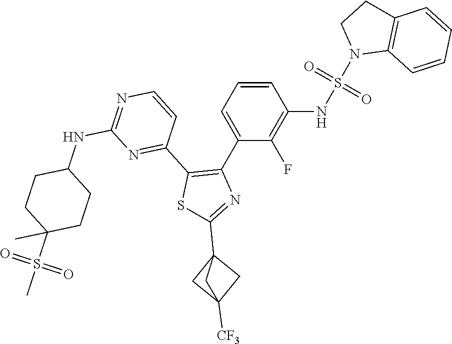 [SMILES: CC1(S(C)(=O)=O)CCC(Nc2nccc(-c3sc(C45CC(C(F)(F)F)(C4)C5)nc3-c3cccc(NS(=O)(=O)N4CCc5ccccc54)c3F)n2)CC1] | N-(2-fluoro- 3-(5-(2-((4- methyl-4- (methylsulfonyl) cyclohexyl)amino) pyrimidin-4- yl)-2-(3- (trifluoromethyl) bicyclo- [1.1.1]pentan- 1-yl)thiazol-4- yl)phenyl)- indoline- 1-sulfonamide | 777.1 |
| 286 | 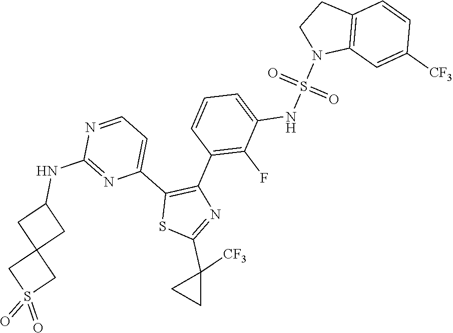 [SMILES: O=S1(=O)CC2(CC(Nc3nccc(-c4sc(C5(C(F)(F)F)CC5)nc4-c4cccc(NS(=O)(=O)N5CCc6ccc(C(F)(F)F)cc65)c4F)n3)C2)C1] | N-(3-(5-(2-((2,2- dioxido-2-thiaspiro- [3.3]heptan-6-yl) amino)pyrimidin-4- yl)- 2-(1-(trifluoromethyl) cyclopropyl)- thiazol-4- yl)-2-fluorophenyl)-6- (trifluoromethyl) indoline- 1-sulfonamide | 789.1 |
| 287 | 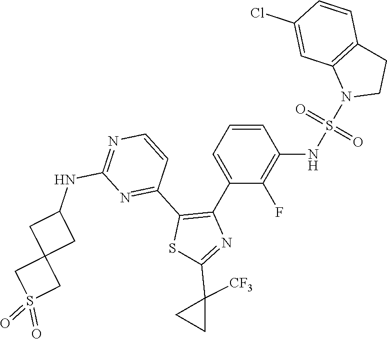 [SMILES: O=S1(=O)CC2(CC(Nc3nccc(-c4sc(C5(C(F)(F)F)CC5)nc4-c4cccc(NS(=O)(=O)N5CCc6ccc(Cl)cc65)c4F)n3)C2)C1] | 6-chloro-N-(3-(5- (2-((2,2-dioxido-2- thiaspiro[3.3]heptan- 6-yl)amino)- pyrimidin-4- yl)-2-(1-(trifluoromethyl)- cyclopropyl)thiazol-4- yl)-2-fluoro- phenyl)indoline- 1-sulfonamide | 755.0 |
| 288 | 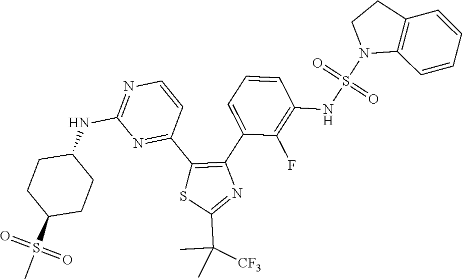 [SMILES: CC(C)(c1nc(-c2cccc(NS(=O)(=O)N3CCc4ccccc43)c2F)c(-c2ccnc(N[C@H]3CC[C@H](S(C)(=O)=O)CC3)n2)s1)C(F)(F)F] | N-(2-fluoro-3- (5-(2-(((1r,4r)-4- (methylsulfonyl) cyclohexyl)amino)- pyrimidin-4- yl)-2-(1,1,1-trifluoro-2- methylpropan- 2-yl)thiazol-4- yl)phenyl)- indoline- 1-sulfonamide | 739.5 |
| 289 | 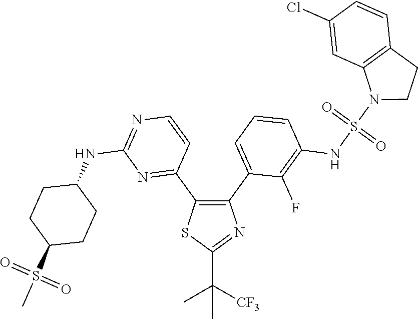 [SMILES: CC(C)(c1nc(-c2cccc(NS(=O)(=O)N3CCc4ccc(Cl)cc43)c2F)c(-c2ccnc(N[C@H]3CC[C@H](S(C)(=O)=O)CC3)n2)s1)C(F)(F)F] | 6-chloro-N-(2- fluoro-3-(5-(2- (((1r,4r)-4- (methylsulfonyl) cyclohexyl)amino) pyrimidin-4- yl)-2-(1,1,1- trifluoro-2- methyl- propan-2-yl)thiazol-4- yl)phenyl)- indoline- 1-sulfonamide | 773.2 |
| 290 | 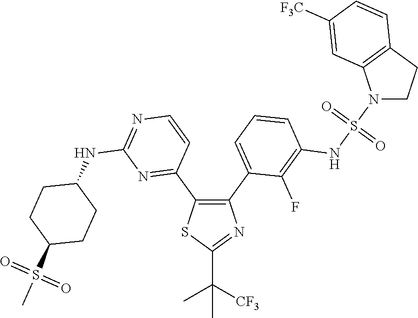 [SMILES: CC(C)(c1nc(-c2cccc(NS(=O)(=O)N3CCc4ccc(C(F)(F)F)cc43)c2F)c(-c2ccnc(N[C@H]3CC[C@H](S(C)(=O)=O)CC3)n2)s1)C(F)(F)F] | N-(2-fluoro-3-(5- (2-(((1r,4r)-4- (methylsulfonyl) cyclohexyl)amino)- pyrimidin-4- yl)-2-(1,1,1- trifluoro-2- methylpropan- 2-yl)thiazol-4- yl)phenyl)- 6-(trifluoromethyl) indoline-1- sulfonamide | 806.9 |
| 291 | 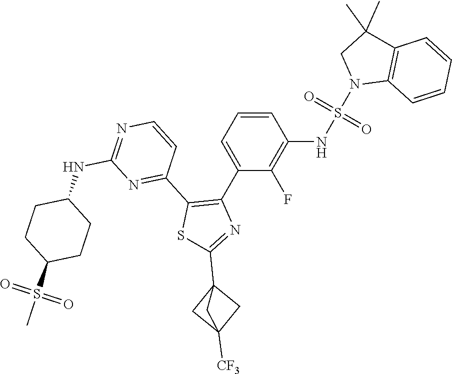 [SMILES: CC1(C)CN(S(=O)(=O)Nc2cccc(-c3nc(C45CC(C(F)(F)F)(C4)C5)sc3-c3ccnc(N[C@H]4CC[C@H](S(C)(=O)=O)CC4)n3)c2F)c2ccccc21] | N-(2-fluoro-3-(5- (2-(((1r,4r)-4- (methylsulfonyl) cyclohexyl)amino) pyrimidin-4- yl)-2-(3- (trifluoromethyl)- bicyclo[1.1.1] pentan- 1-yl)thiazol-4- yl)phenyl)-3,3- dimethylindoline- 1-sulfonamide | 790.9 |
| 292 | 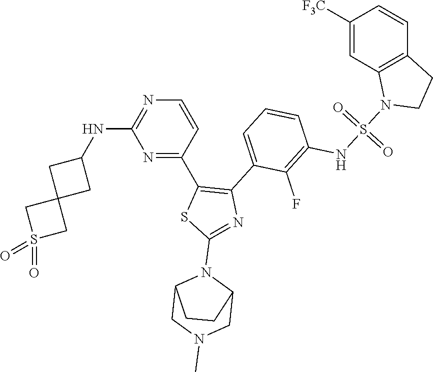 [SMILES: CN1CC2CCC(C1)N2c1nc(-c2cccc(NS(=O)(=O)N3CCc4ccc(C(F)(F)F)cc43)c2F)c(-c2ccnc(NC3CC4(C3)CS(=O)(=O)C4)n2)s1] | N-(3-(5~(2-((2,2- dioxido-2-thiaspiro- [3.3]heptan-6-yl) amino)pyrimidin-4- yl)-2-(3- methyl-3,8- diazabicyclo[3.2.1]- octan-8-yl)thiazol-4- yl)-2-fluorophenyl)- 6-(trifluoromethyl) indoline-1- sulfonamide | 805.4 |
| 293 | 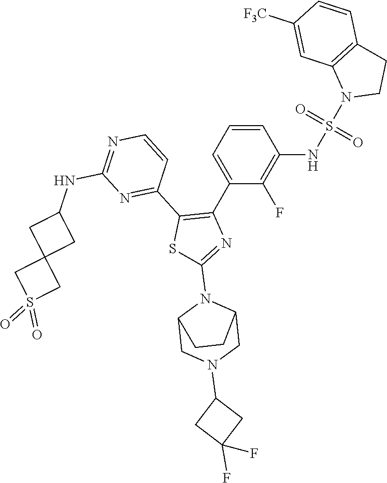 [SMILES: O=S1(=O)CC2(CC(Nc3nccc(-c4sc(N5C6CCC5CN(C5CC(F)(F)C5)C6)nc4-c4cccc(NS(=O)(=O)N5CCc6ccc(C(F)(F)F)cc65)c4F)n3)C2)C1] | N-(3-(2-(3-(3,3- difluorocyclobutyl)-3,8- diazabicyclo[3.2.1] octan-8-yl)-5-(2- ((2,2-dioxido-2- thiaspiro[3.3]heptan-6- yl)amino)pyrimidin-4- yl)thiazol-4- yl)-2- fluorophenyl)- 6-(trifluoromethyl)- indoline- 1-sulfonamide | 880.9 |
| 294 | 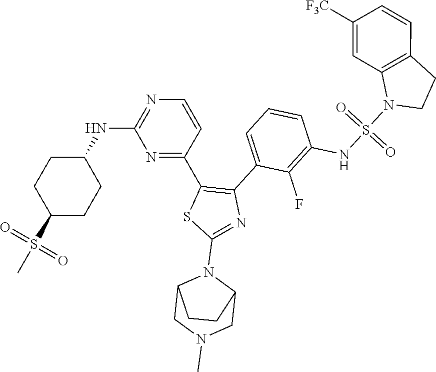 [SMILES: CN1CC2CCC(C1)N2c1nc(-c2cccc(NS(=O)(=O)N3CCc4ccc(C(F)(F)F)cc43)c2F)c(-c2ccnc(N[C@H]3CC[C@H](S(C)(=O)=O)CC3)n2)s1] | N-(2-fluoro- 3-(2-(3- methyl-3,8- diazabicyclo[3.2.1] octan-8-yl)-5-(2- (((1r,4r)-4- (methylsulfonyl) cyclohexyl)- amino)pyrimidin-4- yl)thiazol-4- yl)phenyl)-6- (trifluoromethyl) indoline- 1-sulfonamide | 821.0 |
| 295 | 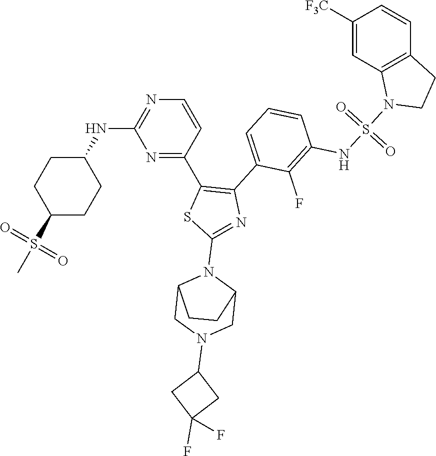 [SMILES: CS(=O)(=O)[C@H]1CC[C@H](Nc2nccc(-c3sc(N4C5CCC4CN(C4CC(F)(F)C4)C5)nc3-c3cccc(NS(=O)(=O)N4CCc5ccc(C(F)(F)F)cc54)c3F)n2)CC1] | N-(3-(2-(3-(3,3- difluorocyclobutyl)- 3,8- diazabicyclo[3.2.1] octan-8-yl)-5-(2- (((1r,4r)-4- (methylsulfonyl) cyclohexyl)- amino)pyrimidin-4- yl)thiazol-4- yl)-2- fluorophenyl)-6- (trifluoromethyl)- indoline- 1-sulfonamide | 896.9 |
| 296 | 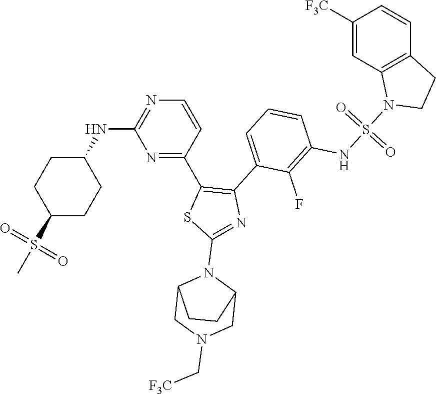 [SMILES: CS(=O)(=O)[C@H]1CC[C@H](Nc2nccc(-c3sc(N4C5CCC4CN(CC(F)(F)F)C5)nc3-c3cccc(NS(=O)(=O)N4CCc5ccc(C(F)(F)F)cc54)c3F)n2)CC1] | N-(2-fluoro-3-(5- (2-(((1r,4r)-4- (methylsulfonyl) cyclohexyl)amino)- pyrimidin-4- yl)-2-(3-(2,2,2- trifluoroethyl)-3, 8-diazabicyclo[3.2.1]- octan-8-yl)thiazol-4- yl)phenyl)-6- (trifluoromethyl) indoline- 1-sulfonamide | 889.3 |
| 297 | 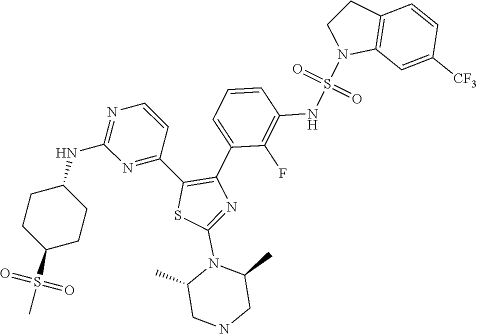 [SMILES: C[C@H]1CNC[C@H](C)N1c1nc(-c2cccc(NS(=O)(=O)N3CCc4ccc(C(F)(F)F)cc43)c2F)c(-c2ccnc(N[C@H]3CC[C@H](S(C)(=O)=O)CC3)n2)s1] | N-(3-(2-((38, 5S)-3,5-dimethyl- morpholino)-5- (2-(((1r,4S)-4- (methyl- sulfonyl) cyclohexyl) amino) pyrimidin-4- yl)thiazol-4- yl)-2~fluorophenyl)-6- (trifluoromethyl) indoline- 1-sulfonamide | 810.2 |
| 298 | 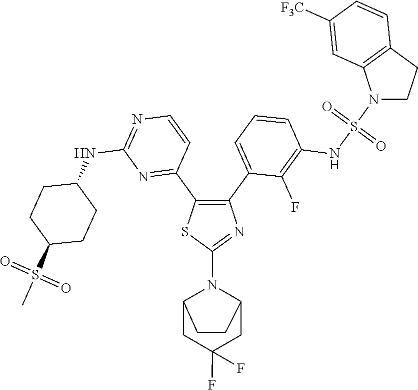 [SMILES: CS(=O)(=O)[C@H]1CC[C@H](Nc2nccc(-c3sc(N4C5CCC4CC(F)(F)C5)nc3-c3cccc(NS(=O)(=O)N4CCc5ccc(C(F)(F)F)cc54)c3F)n2)CC1] | N-(3-(2-(3,3-difluoro- 8-azabicyclo- [3.2.1]octan-8-yl)- 5-(2-(((1r,4r)-4- (methylsulfonyl) cyclohexyl)amino)- pyrimidin-4- yl)thiazol-4- yl)-2-fluoro- phenyl)-6- (trifluoromethyl)- indoline-1- sulfonamide | 841.9 |
| 299 | 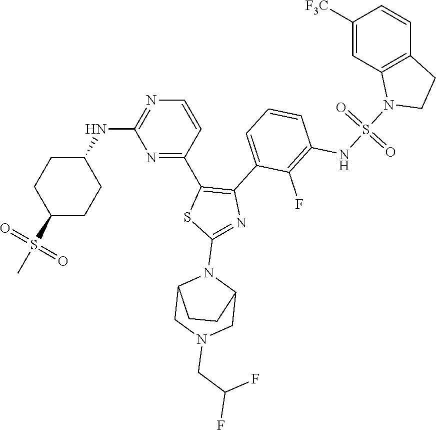 [SMILES: CS(=O)(=O)[C@H]1CC[C@H](Nc2nccc(-c3sc(N4C5CCC4CN(CC(F)F)C5)nc3-c3cccc(NS(=O)(=O)N4CCc5ccc(C(F)(F)F)cc54)c3F)n2)CC1] | N-(3-(2-(3-(2,2- difluoroethyl)-3,8- diazabicyclo[3.2.1] octan-8-yl)-5-(2- (((1r,4r)-4- (methylsulfonyl) cyclohexyl)- amino)pyrimidin-4- yl)thiazol-4-yl)-2- fluorophenyl)- 6-(trifluoromethyl)- indoline- 1-sulfonamide | 871.2 |

### EMBODIMENTS

In further embodiments 1-203 below, the present disclosure includes:

1. In embodiment 1, the compound of Formula (I), or a pharmaceutically acceptable salt thereof, is as provided in the first aspect of the Summary above.

2. In embodiment 2, the compound is as provided in embodiment 1, or a pharmaceutically acceptable salt thereof, wherein:

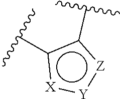

is:

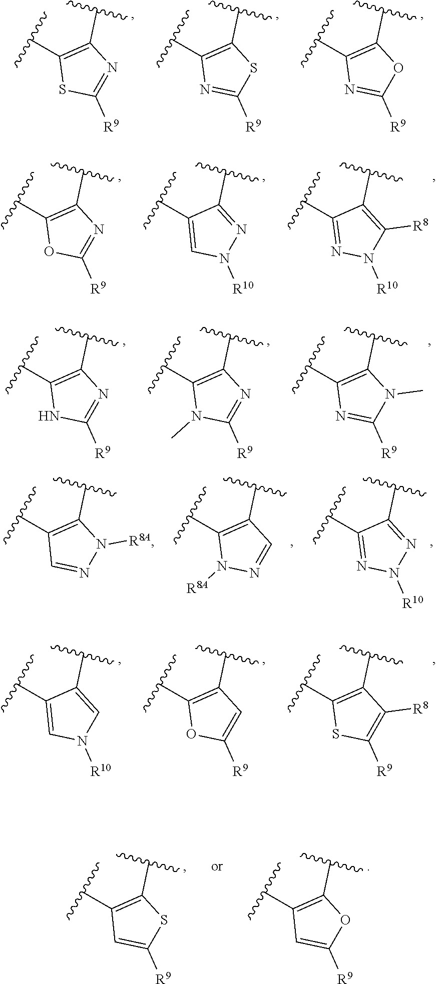

3. In embodiment 3, the compound of Formula (I) is as provided in embodiment 1 or 2, or a pharmaceutically acceptable salt thereof, wherein:

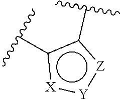

is

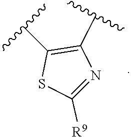

4. In embodiment 4, the compound is as provided in embodiment 1 or 2, or a pharmaceutically acceptable salt thereof, wherein;

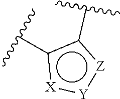

is

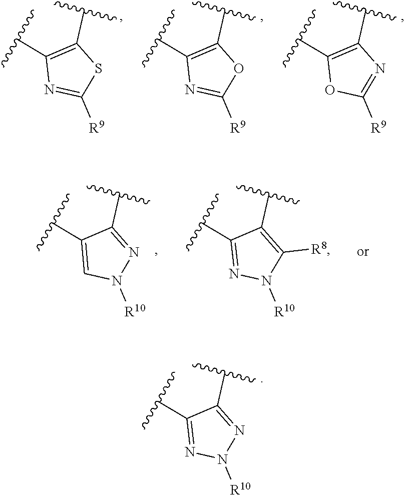

4a. In embodiment 4a, the compound is as provided in embodiment 4, or a pharmaceutically acceptable salt thereof, wherein R⁸ is hydrogen, halo, or cyano.

5. In embodiment 5, the compound is as provided in embodiment 1, 2, or 4, or a pharmaceutically acceptable salt thereof, wherein:

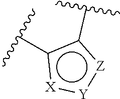

is

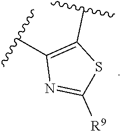

6. In embodiment 6, the compound is as provided in embodiment 1, 2, or 4, or a pharmaceutically acceptable salt thereof, wherein:

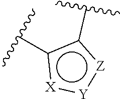

is

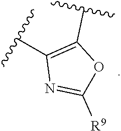

7. In embodiment 7, the compound is as provided in embodiment 1, 2, or 4, or a pharmaceutically acceptable salt thereof, wherein:

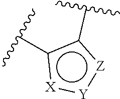

is

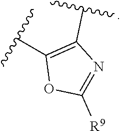

8. In embodiment 8, the compound is as provided in embodiment 1, 2, 4, or 4a, or a pharmaceutically acceptable salt thereof, wherein:

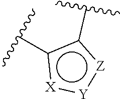

is

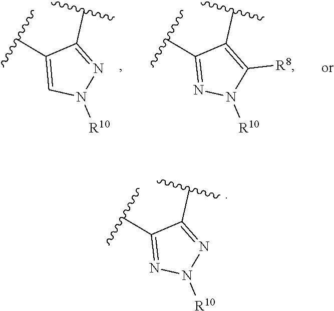

9. In embodiment 9, the compound is as provided in embodiment 1 or 2, or a pharmaceutically acceptable salt thereof, wherein:

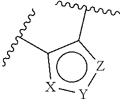

is

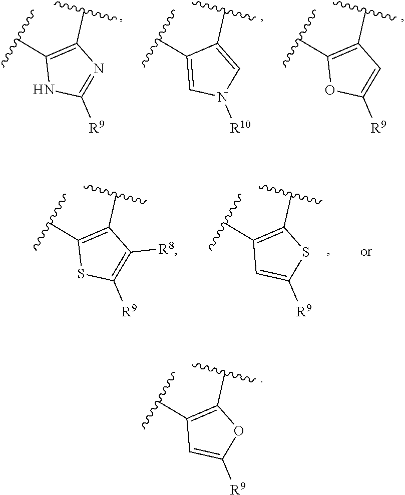

9a. In embodiment 9a, the compound is as provided in embodiment 9, or a pharmaceutically acceptable salt thereof, wherein R⁸ is hydrogen, halo, or cyano.

10. In embodiment 10, the compound is as provided in embodiment 1 or 2, or a pharmaceutically acceptable salt thereof, wherein:

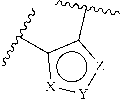

is

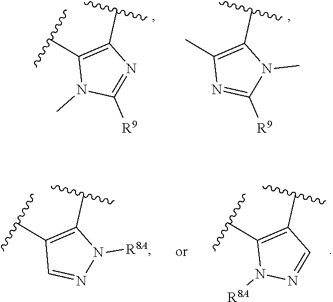

11. In embodiment 11, the compound of any one of embodiments 1 to 10, or a pharmaceutically acceptable salt thereof, has a structure according to formula (Ia):

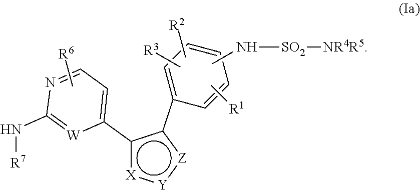

12. In embodiment 8, the compound of any one of embodiments 1 to 11, or a pharmaceutically acceptable salt thereof, has a structure according to formula (Ib):

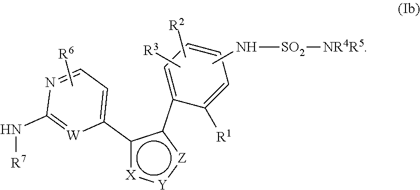

13. In embodiment 13, the compound of any one of embodiments 1 to 11, or a pharmaceutically acceptable salt thereof, has a structure according to formula (Ic):

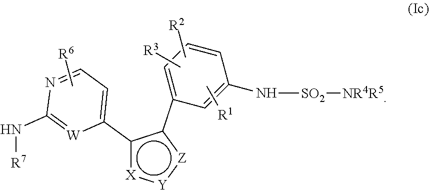

14. In embodiment 14, the compound of any one of embodiments 1 to 13, or a pharmaceutically acceptable salt thereof, has a structure according to formula (Id):

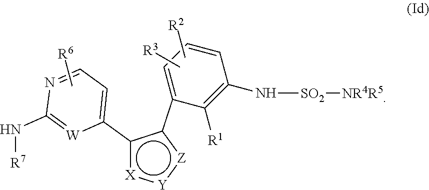

15. In embodiment 15, the compound of any one of embodiments 1 to 14, or a pharmaceutically acceptable salt thereof, has a structure according to formula (Ie):

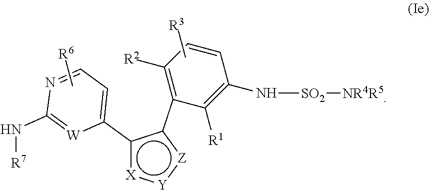

16. In embodiment 16, the compound is as provided in any one of embodiments 1 to 10, or a pharmaceutically acceptable salt thereof, wherein ring R^{A} is heteroaryl.

17. In embodiment 17, the compound is as provided in any one of embodiments 1 to 10 and 16, or a pharmaceutically acceptable salt thereof, wherein ring R^{A} is a six-membered heteroaryl containing one or two nitrogens.

17a. In embodiment 17a, the compound is as provided in any one of embodiments 1 to 10, 16, and 17, or a pharmaceutically acceptable salt thereof, wherein ring R^{A} is pyridyl, pyrimidinyl, pyrazinyl, or pyridazinyl.

17b. In embodiment 17b, the compound of any one of embodiments 1 to 10, 16, and 17, or a pharmaceutically acceptable salt thereof, has a structure according to formula (If), (Ig), (Ih), (Ii), (Ij), or (Ik):

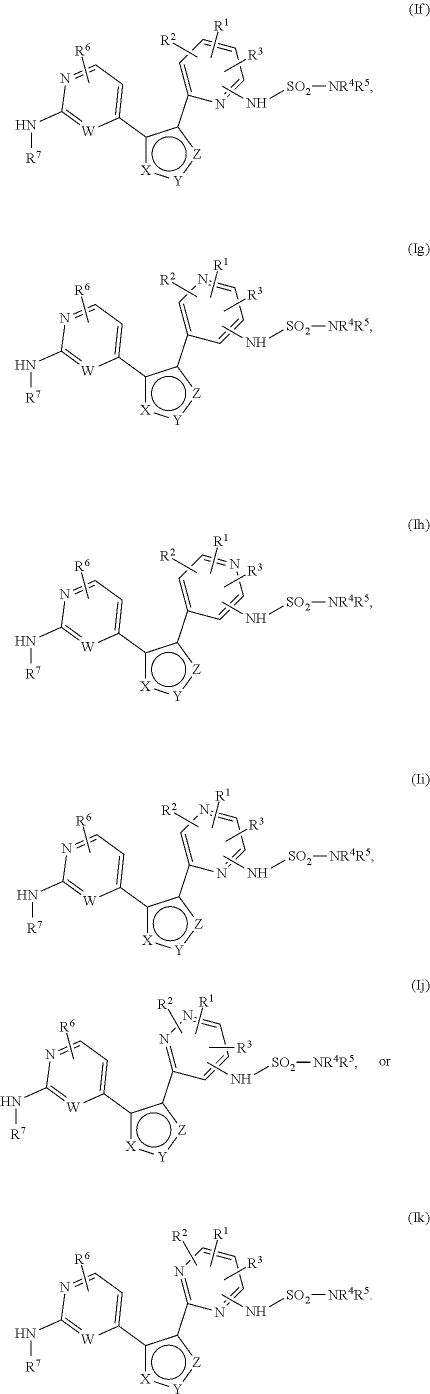

18. In embodiment 18, the compound of any one of embodiments 1 to 10 and 16, or a pharmaceutically acceptable salt thereof, is wherein ring R^{A} is a bicyclic heteroaryl.

18a. In embodiment 18a, the compound of any one of embodiments 1 to 10 and 18, or a pharmaceutically acceptable salt thereof, has a structure according to formula (Im), (In), (Io), (Ip), or (Iq):

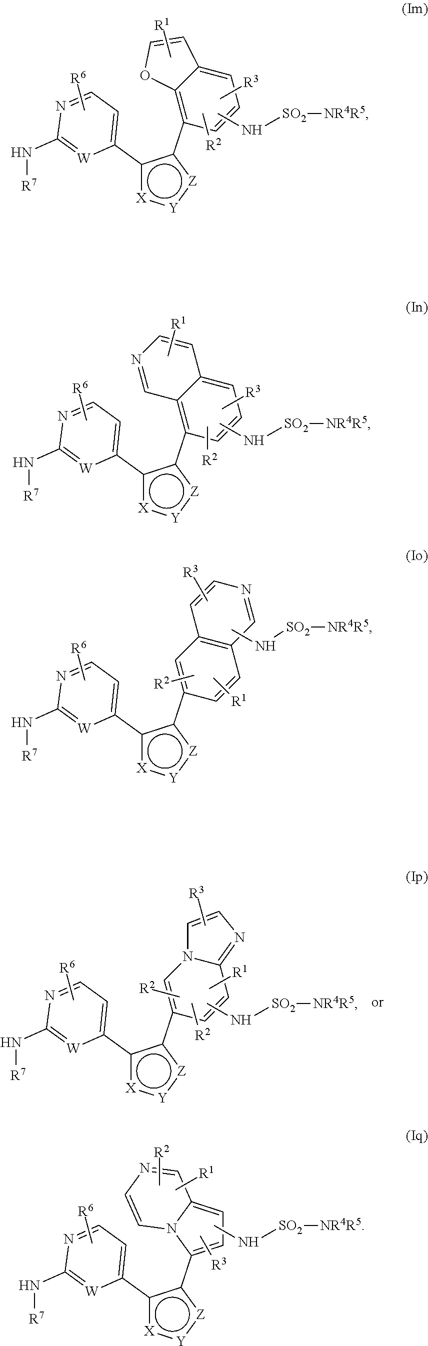

19. In embodiment 19, the compound is as provided in any one of embodiments 1 to 18a, or a pharmaceutically acceptable salt thereof, wherein R¹, R², and R³ are independently hydrogen, deuterium, alkyl, halo, haloalkyl, haloalkoxy, alkoxy, hydroxy, or cyano.

20. In embodiment 20, the compound is as provided in any one of embodiments 1 to 18a, or a pharmaceutically acceptable salt thereof, wherein R¹, R², and R³ are independently selected from hydrogen, deuterium, methyl, ethyl, propyl, fluoro, chloro, bromo, difluoromethyl, trifluoromethyl, difluoromethoxy, trifluoromethoxy, methoxy, ethoxy, hydroxy, hydroxymethyl, methoxymethyl, cyano, and —CH₂NH₂.

21. In embodiment 21, the compound is as provided in any one of embodiments 1 to 20, or a pharmaceutically acceptable salt thereof, wherein R¹, R², and R³ are independently hydrogen, deuterium, methyl, ethyl, propyl, fluoro, chloro, bromo, difluoromethyl, trifluoromethyl, difluoromethoxy, trifluoromethoxy, methoxy, ethoxy, hydroxy, or cyano.

22. In embodiment 22, the compound is as provided in any one of embodiments 1 to 21, or a pharmaceutically acceptable salt thereof, wherein R¹ and R² are independently hydrogen, deuterium, fluoro, chloro, difluoromethyl, trifluoromethyl, difluoromethoxy, trifluoromethoxy, methoxy, hydroxy, or cyano and R³ is hydrogen.

23 In embodiment 23, the compound is as provided in any one of embodiments 1 to 22, or a pharmaceutically acceptable salt thereof, wherein R¹ is fluoro, R² is hydrogen, deuterium, fluoro, chloro, difluoromethyl, trifluoromethyl, difluoromethoxy, trifluoromethoxy, methoxy, hydroxy, or cyano and R³ is hydrogen.

23a. In embodiment 23a, the compound is as provided in any one of embodiments 1 to 23, or a pharmaceutically acceptable salt thereof, wherein R¹ is fluoro, R² is hydrogen, deuterium, fluoro, chloro, difluoromethyl, trifluoromethyl, difluoromethoxy, trifluoromethoxy, or cyano, and R³ is hydrogen.

24. In embodiment 24, the compound is as provided in any one of embodiments 1 to 23, or a pharmaceutically acceptable salt thereof, is wherein R¹ is fluoro and R² and Rare hydrogen.

25. In embodiment 25, the compound of any one of embodiments 1 to 24, or a pharmaceutically acceptable salt thereof, wherein R⁴ and R⁵ together with the nitrogen atom to which they are attached form a ring selected from cyclylaminyl, bicyclylaminyl, fused cyclylaminyl, bridged cyclylaminyl, or spiroaminyl, wherein each of the aforementioned rings is substituted with R^{s}, R^{t}, and R^{u}.

26. In embodiment 26, the compound is as provided in any one of embodiments 1 to 24, or a pharmaceutically acceptable salt thereof, wherein R⁴ and R⁵ together with the nitrogen atom to which they are attached form a ring selected from fused bridged cyclylaminyl and fused spiroaminyl, wherein each of the aforementioned rings is substituted with R^{s}, R^{t}, and R^{u}.

27. In embodiment 27, the compound is as provided in any one of embodiments 1 to 25, or a pharmaceutically acceptable salt thereof, wherein R⁴ and R⁵ together with the nitrogen atom to which they are attached form a ring selected from cyclylaminyl, bicyclylaminyl, and fused cyclylaminyl, wherein each of the aforementioned rings is substituted with R^{s}, R^{t}, and R^{u}.

28. In embodiment 28, the compound is as provided in any one of embodiments 1 to 25, or a pharmaceutically acceptable salt thereof, wherein R⁴ and R⁵ together with the nitrogen atom to which they are attached form a ring selected from bridged cyclylaminyl and spiroaminyl, wherein each of the aforementioned rings is substituted with R^{s}, R^{t}, and R^{u}.

29. In embodiment 29, the compound is as provided in any one of embodiments 1 to 25 and 27, or a pharmaceutically acceptable salt thereof, wherein R⁴ and R⁵ together with the nitrogen atom to which they are attached form fused cyclylaminyl substituted with R^{s}, R^{t}, and R^{u}.

30. In embodiment 30, the compound is as provided in any one of embodiments 1 to 25 and 27, or a pharmaceutically acceptable salt thereof, wherein R⁴ and R⁵ together with the nitrogen atom to which they are attached form cyclylaminyl or bicyclylaminyl, wherein each of the aforementioned rings is substituted with R^{s}, R^{t}, and R^{u}.

31. In embodiment 31, the compound is as provided in any one of embodiments 1 to 25, 27, and 29, or a pharmaceutically acceptable salt thereof, wherein the fused cyclylaminyl formed by R⁴ and R⁵ together with the nitrogen atom to which they are attached is where two adjacent ring atoms of cyclylaminyl are fused to two adjacent atoms of phenyl and is substituted with R^{s}, R^{t}, and R^{u}.

32. In embodiment 32, the compound is as provided in any one of embodiments 1 to 25, 27, and 29, or a pharmaceutically acceptable salt thereof, wherein the fused cyclylaminyl formed by R⁴ and R⁵ together with the nitrogen atom to which they are attached is where two adjacent ring atoms of cyclylaminyl are fused to two adjacent atoms of a five- or six-membered heteroaryl and is substituted with R^{s}, R^{t}, and R^{u}.

33. In embodiment 33, the compound is as provided in any one of embodiments 1 to 25, 27, 29, and 31, or a pharmaceutically acceptable salt thereof, wherein the fused cyclylaminyl formed by R⁴ and R⁵ together with the nitrogen atom to which they are attached has a structure according to formula (a):

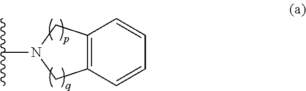

where:
p is 0, 1, or 2 and q is 0, 1, 2, 3, or 4, provided p+q is at least 2 and p+q is not more than 5; and when p or q is 3 or 4, then one of the —CH₂— of such p or q can be replaced by NH, N (when attached to one of R^{s}, R^{t}, and R^{u}), O or S(O)ₙ; wherein n is 0, 1, or 2; and
ring (a) is substituted with R^{s}, R^{t}, and R^{u}.

34. In embodiment 34, the compound is as provided in any one of embodiments 1 to 25, 27, 29, and 32, or a pharmaceutically acceptable salt thereof, wherein the fused cyclylaminyl formed by R⁴ and R⁵ together with the nitrogen atom to which they are attached has a structure according to formula (b):

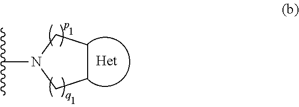

where:
p₁ is 0, 1, or 2 and q₁ is 0, 1, 2, 3, or 4, provided p+q is at least 2 and p+q is not more than 5; and when p or q is 3 or 4, then one of the —CH₂— of such p or q can be replaced by NH, N (when attached to one of R^{s}, R^{t}, and R^{u}), O or S(O)ₙ; wherein n is 0, 1, or 2;
Het is 5- or 6-membered heteroaryl; and
ring (b) is substituted with R^{s}, R^{t}, and R^{u}.

35. In embodiment 35, the compound is as provided in any one of embodiments 1 to 34, or a pharmaceutically acceptable salt thereof, wherein R⁴ and R⁵ together with the nitrogen atom to which they are attached form a ring selected form:

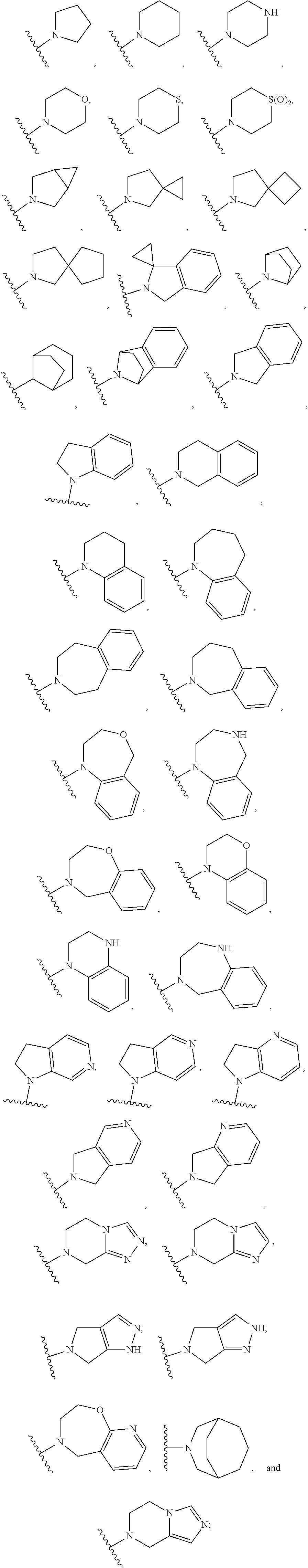

wherein each ring is substituted with R^{s}, R^{t}, and R^{u}.

36. In embodiment 36, the compound is as provided in any one of embodiments 1 to 25, 27, 29, and 31 to 35, or a pharmaceutically acceptable salt thereof, is wherein the fused cyclylaminyl formed by R⁴ and R⁵ together with the nitrogen atom to which they are attached is selected from:

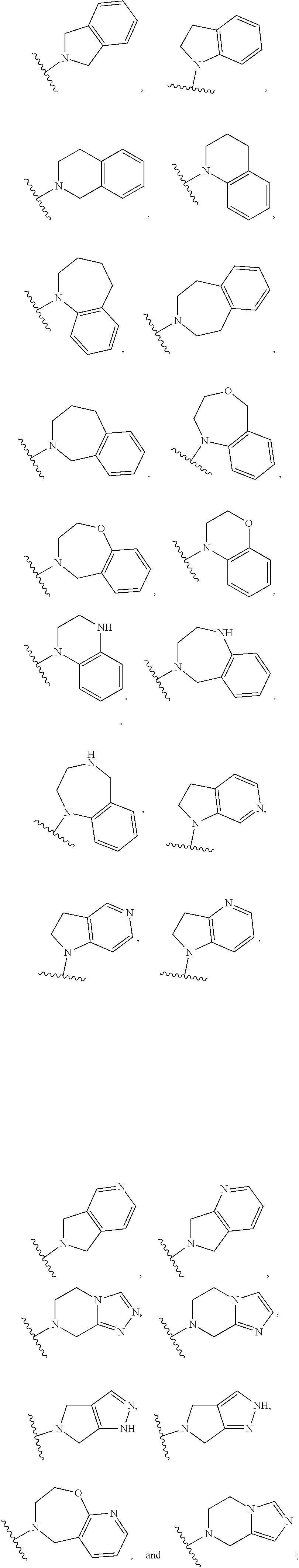

wherein each ring is substituted with R^{s}, R^{t}, and R^{u}.

37. In embodiment 37, the compound is as provided in any one of embodiments 1 to 25, 27, 29, 31, 33, 35 and 36, or a pharmaceutically acceptable salt thereof, wherein the fused cyclylaminyl formed by R⁴ and R⁵ together with the nitrogen atom to which they are attached is selected from:

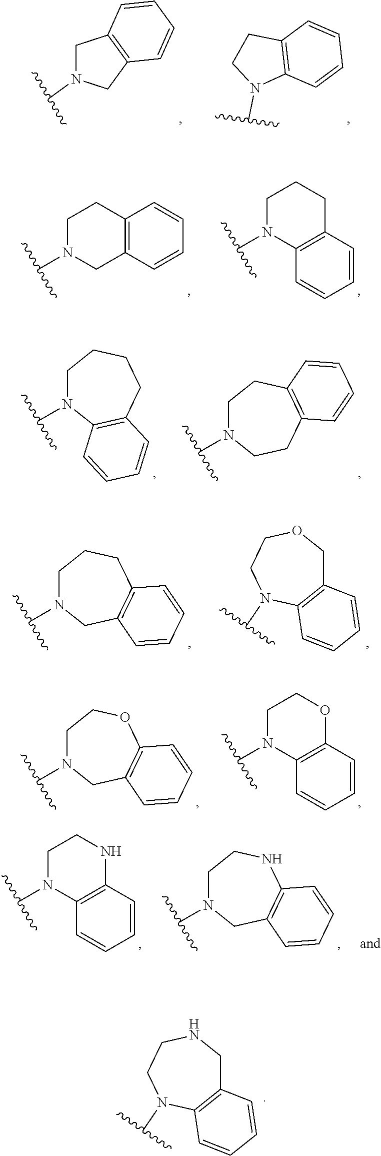

each ring substituted with R^{s}, R^{t}, and R^{u}.

38. In embodiment 38, the compound is as provided in any one of embodiments 1 to 25, 27, 29, 32, and 34 to 36, or a pharmaceutically acceptable salt thereof, wherein the fused cyclylaminyl formed by R⁴ and R⁵ together with the nitrogen atom to which they are attached is selected from:

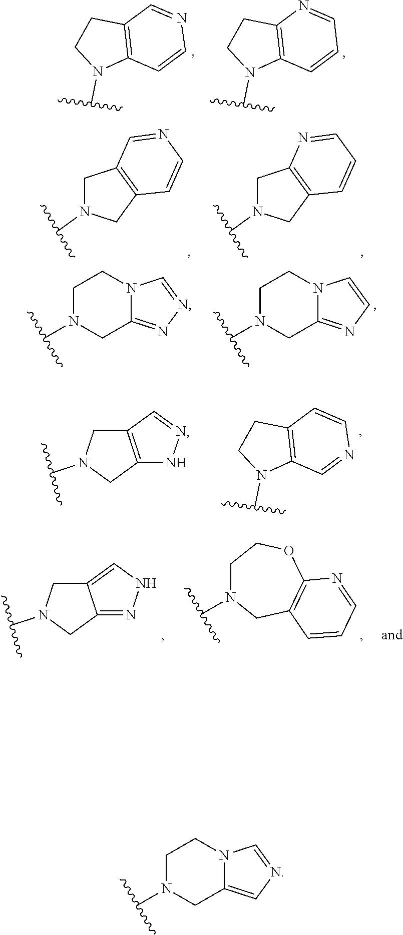

wherein each ring is substituted with R^{s}, R^{t}, and R^{u}.

39. In embodiment 39, the compound is as provided in any one of embodiments 1 to 25, 27, 29, 31, 33, and 35 to 37, or a pharmaceutically acceptable salt thereof, wherein the fused cyclylaminyl formed by R⁴ and R⁵ together with the nitrogen atom to which they are attached is selected from:

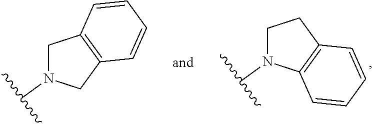 [SMILES: *N1Cc2ccccc2C1 ; *N1CCc2ccccc21]

wherein each ring is substituted with R^{s}, R^{t}, and R^{u}.

40. In embodiment 40, the compound is as provided in any one of embodiments 1 to 39, or a pharmaceutically acceptable salt thereof, wherein R^{u} is hydrogen.

41. In embodiment 41, the compound is as provided in any one of embodiments 1 to 25, 27, 29, and 31 to 40, or a pharmaceutically acceptable salt thereof, wherein the phenyl and 5- or 6-membered heteroaryl portion of fused cyclylaminyl formed by R⁴ and R⁵ together with the nitrogen atom to which they are attached are substituted with R^{s} and R^{t}.

42. In embodiment 42, the compound is as provided in any one of embodiments 1 to 25, 27, 29, 31, 33, and 35 to 37, or a pharmaceutically acceptable salt thereof, wherein the fused cyclylaminyl formed by R⁴ and R⁵ together with the nitrogen atom to which they are attached is a ring of formula (a1) or (a2):

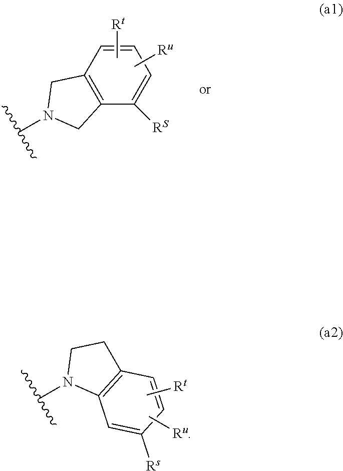

43. In embodiment 43, the compound is as provided in any one of embodiments 1 to 39, and 42, or a pharmaceutically acceptable salt thereof, wherein R^{s}, R^{t}, and R^{u} are independently selected from hydrogen, deuterium, alkyl, alkoxy, alkylsulfonyl, hydroxy, acyl, halo, haloalkyl, haloalkoxy, amino, alkylamino, dialkylamino, substituted amino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy and unsaturated heterocyclyl (wherein aryl, by itself or as part of aryloxy and aralkyl, heteroaryl, by itself or as part of heteroaryloxy and heteroaralkyl, heterocyclyl, by itself or as part of heterocyclylalkyl and heterocyclyloxy and unsaturated heterocyclyl are substituted with one to three substituents independently selected from hydrogen, alkyl, alkoxy, hydroxy, halo, haloalkyl, haloalkoxy, and cyano).

44. In embodiment 44, the compound is as provided in any one of embodiments 1 to 39, 42, and 43, or a pharmaceutically acceptable salt thereof, wherein R^{s} is hydrogen, deuterium, alkyl, alkoxy, alkylsulfonyl, hydroxy, acyl, halo, haloalkyl, haloalkoxy, amino, alkylamino, dialkylamino, substituted amino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, or unsaturated heterocyclyl (wherein aryl, by itself or as part of aryloxy and aralkyl, heteroaryl, by itself or as part of heteroaryloxy and heteroaralkyl, heterocyclyl, by itself or as part of heterocyclylalkyl and heterocyclyloxy and unsaturated heterocyclyl are substituted with one to three substituents independently selected from hydrogen, alkyl, alkoxy, hydroxy, halo, haloalkyl, haloalkoxy, and cyano) and R^{t} and R^{u} are independently selected from hydrogen, alkyl, alkoxy, alkylsulfonyl, hydroxy, acyl, halo, haloalkyl, haloalkoxy, amino, alkylamino, dialkylamino, and cyano.

45. In embodiment 45, the compound is as provided in any one of embodiments 1 to 39 and 42 to 44, or a pharmaceutically acceptable salt thereof, wherein R^{s} is hydrogen, deuterium, alkyl, alkoxy, alkylsulfonyl, hydroxy, acyl, halo, haloalkyl, haloalkoxy, amino, alkylamino, dialkylamino, substituted amino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, or unsaturated heterocyclyl (wherein aryl, by itself or as part of aryloxy and aralkyl, heteroaryl, by itself or as part of heteroaryloxy and heteroaralkyl heterocyclyl, by itself or as part of heterocyclylalkyl and heterocyclyloxy and unsaturated heterocyclyl are substituted with one to three substituents independently selected from hydrogen, alkyl, alkoxy, hydroxy, halo, haloalkyl, haloalkoxy, and cyano), R^{t} is hydrogen, alkyl, alkoxy, alkylsulfonyl, hydroxy, acyl, halo, haloalkyl, haloalkoxy, amino, alkylamino, dialkylamino, and cyano, and R^{u} is hydrogen.

46. In embodiment 46, the compound is as provided in any one of embodiments 1 to 45, or a pharmaceutically acceptable salt thereof, wherein R^{s} is hydrogen, deuterium, alkyl, alkoxy, alkylsulfonyl, hydroxy, acyl, halo, haloalkyl, haloalkoxy, amino, alkylamino, dialkylamino, alkoxycarbonylaminoalkyl, aminocarbonyl, cyano, hydroxyalkyl, hydroxyalkoxy, aminoalkyl, aminoalkoxy, cyanoalkyl, cyanoalkoxy, cycloalkyl, aryl, aryloxy, aralkyl, heteroaryl, heterocyclylalkyl, or unsaturated heterocyclyl (wherein aryl, by itself or as part of aryloxy and aralkyl, heteroaryl, heterocyclyl as part of heterocyclylalkyl, and unsaturated heterocyclyl are substituted with one to three substituents independently selected from hydrogen, alkyl, alkoxy, hydroxy, halo, haloalkyl, haloalkoxy, and cyano), R^{t} is hydrogen, alkyl, alkoxy, hydroxy, halo, haloalkyl, or haloalkoxy and R^{u} is hydrogen.

47. In embodiment 47, the compound is as provided in any one of embodiments 1 to 46, or a pharmaceutically acceptable salt thereof, wherein R^{s} is selected from hydrogen, deuterium, methyl, ethyl, isopropyl, cyclopropyl, cyclohexyl, methoxy, ethoxy, isopropoxy, methylsulfonyl, ethylsulfonyl, hydroxy, methylcarbonyl, fluoro, chloro, difluoromethyl, trifluoromethyl, difluoromethoxy, trifluoromethoxy, amino, methylamino, dimethylamino, cyano, cyanomethyl, 2-cyanoprop-2-yl, cyanomethyloxy, hydroxymethyl, 1-hydroxyethyl, 1-hydroxy-1-methylethyl, methoxymethyl, methylaminomethyl, dimethylaminomethyl, methoxycarbonylaminomethyl, —CONH₂, methylaminocarbonyl, dimethylaminocarbonyl, 2-hydroxyethyloxy, —O—(CH₂)₂NH₂, 2-methylaminoethyloxy, 2-dimethylaminoethyloxy, phenyl, phenoxy, 3-fluorophenoxy, 4-fluorophenoxy, 2-cyanophenoxy, 3-cyanophenyl, benzyl, 1-methyl-2-oxo-1,6-dihydropyridinyl, and morpholin-4-ylmethyl, R^{t} is hydrogen, fluoro, chloro, methoxy, difluoromethyl, difluoromethoxy, trifluoromethyl, trifluoromethoxy, or cyano, and R^{u} is hydrogen.

48. In embodiment 48, the compound is as provided in any one of embodiments 1 to 47, or a pharmaceutically acceptable salt thereof, wherein R^{s} is selected from hydrogen, methyl, ethyl, isopropyl, cyclopropyl, cyclohexyl, methoxy, ethoxy, isopropoxy, methylsulfonyl, ethylsulfonyl, hydroxy, methylcarbonyl, fluoro, chloro, difluoromethyl, trifluoromethyl, difluoromethoxy, trifluoromethoxy, amino, methylamino, dimethylamino, cyano, cyanomethyl, 2-cyanoprop-2-yl, cyanomethyloxy, hydroxymethyl, 1-hydroxyethyl, 1-hydroxy-1-methylethyl, methoxymethyl, methylaminomethyl, dimethylaminomethyl, methoxycarbonylaminomethyl, —CONH₂, methylaminocarbonyl, dimethylaminocarbonyl, 2-hydroxyethloxy, —O—(CH₂)₂NH₂, 2-methylaminoethyloxy, 2-dimethylaminoethyloxy, phenyl, phenoxy, 3-fluorophenoxy, 4-fluorophenoxy, 2-cyanophenoxy, 3-cyanophenyl, benzyl, 1-methyl-2-oxo-1,6-dihydropyridinyl, and morpholin-4-ylmethyl and R^{t} and R^{u} are hydrogen.

49. In embodiment 49, the compound is as provided in any one of embodiments 1 to 48, or a pharmaceutically acceptable salt thereof, wherein R⁴ and R⁵ together with the nitrogen atom to which they are attached form a ring selected from morpholin-4-yl, piperidin-1-yl, 2-methylpiperidin-1-yl, 3-methylpiperidin-1-yl, 4-methylpiperidin-1-yl, 4-phenoxypiperidin-1-yl, 2-phenylpiperidin-1-yl, 2-phenylpyrrolidin-1-yl, 3,3-difluoropyrrolidin-1-yl, 4-phenylpiperazin-1-yl, 4-acetylpiperazin-1-yl, 4-methyl-3-oxo-piperazin-1-yl, 4-methylsulfonylpiperazin-1-yl, 3-azabicyclo[3.1.0]hexan-3-yl, azaspiro[2.4]heptan-5-yl, 2-azaspiro[4.4]nonan-2-yl, 6-azaspiro[3.4]octan-6-yl, 5-azaspiro[2.4]heptan-5-yl, 7-azabicyclo[2.2.1]heptan-7-yl, 8-azabicyclo[3.2.1]octan-8-yl, 2-azabicyclo[2.2.2]octan-2-yl, 3-azabicyclo[3.2.2]nonan-3-yl, isoindolin-2-yl, 4-cyanoisoindolin-2-yl, S-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, 5-hydroxymethylisoindolin-2-yl, 4-difluoromethylisoindolin-2-yl, 5-difluoromethylisoindolin-2-yl, 4-difluoromethoxyisoindolin-2-yl, 4-trifluoromethylisoindolin-2-yl, 5-trifluoromethylisoindolin-2-yl, 4-fluoroisoindolin-2-yl, 5-fluoroisoindolin-2-yl, 5,6-difluoroisoindolin-2-yl, 1-methylisoindoline-2-yl, 4-methylamino-carbonylisoindolin-2-yl, 4-dimethylaminocarbonylisoindolin-2-yl, 5-dimethylaminocarbonyl-isoindolin-2-yl, 4-(2-(dimethylamino)ethoxy)isoindolin-2-yl, 4-(2-(methylamino)ethoxy)-isoindolin-2-yl, 4-dimethylaminomethylisoindolin-2-yl, 5-dimethylaminomethylisoindolin-2-yl, 4-methylaminomethylisoindolin-2-yl, 4-dimethylaminoisoindolin-2-yl, 4-methylaminoisoindolin-2-yl, 3-phenylisoindolin-2-yl, 4-phenylisoindolin-2-yl, 4-phenoxyisoindolin-2-yl, 1-benzylisoindolin-2-yl, 4-benzylisoindolin-2-yl, 4-(3-fluorophenoxy)isoindolin-2-yl, 4-(4-fluorophenoxy)isoindolin-2-yl, 4-(2-cyanophenoxy)-isoindolin-2-yl, 4-(3-cyanophenoxy)-isoindolin-2-yl, 4-cyclohexylisoindolin-2-yl, 5-(hydroxymethyl)isoindolin-2-yl, 4-(2-hydroxyethoxy)isoindolin-2-yl, 4-(2-aminoethoxy)-isoindolin-2-yl, 5-(cyanomethyl)isoindolin-2-yl, 4-(cyanomethyl)isoindolin-2-yl, indolin-1-yl, 2-methylindolin-1-yl, 4-cyanoindolin-1-yl, 5-cyanoindolin-1-yl, 6-cyanoindolin-1-yl, 4-hydroxy-methylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloro-indolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxyindolin-1-yl, 6-trifluoromethoxylindolin-1-yl, 4-((dimethylamino)methyl)-indolin-1-yl, 5-((dimethyl-amino)methyl)indolin-1-yl, 6-((dimethylamino)methyl)indolin-1-yl, 4-((methylamino)-methyl)indolin-1-yl, 5-((methylamino)methyl)indolin-1-yl, 6-((methylamino)-methyl)indolin-1-yl, 6-methylaminocarbonylindolin-1-yl, 6-aminocarbonyl-indolin-1-yl, 6-dimethylamino-carbonylindolin-1-yl, 4-(2-(methylamino)ethoxy) indolin-1-yl, 6-(2-(dimethylamino)ethoxy)-indolin-1-yl, 4 (hydroxymethyl)indolin-1-yl, 5-phenylindolin-1-yl, 6-(1-methyl-6-oxo-1,6-dihydropyridin-2-yl)indolin-1-yl, 4-(1-hydroxyethyl)indolin-1-yl, 4-(2-hydroxypropan-2-yl)-indolin-1-yl, 6-(2-hydroxypropan-2-yl)indolin-1-yl, 4-(cyanomethyl)indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, 6-chloroindolin-1-yl, 3,4-dihydroisoquinolin-2(1H)-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepin-2-yl, 1-methyl-1,2,3,5-tetrahydro-4H-benzo[e][1,4]diazepin-4-yl, and 2,3-dihydrobenzo[e][1,4]oxazepin-1(5H)-yl.

50. In embodiment 50, the compound is as provided in any one of embodiments 1 to 49, or a pharmaceutically acceptable salt thereof, wherein R⁴ and R⁵ together with the nitrogen atom to which they are attached form a ring selected from morpholin-4-yl, piperidin-1-yl, 2-methyl-piperidin-1-yl, 3-methylpiperidin-1-yl, 4-methylpiperidin-1-yl, 4-phenoxypiperidin-1-yl, 2-phenylpiperidin-1-yl, 2-phenylpyrrolidin-1-yl, 3,3-difluoropyrrolidin-1-yl, 4-phenylpiperazin-1-yl, 4-acetylpiperazin-1-yl, 4-methyl-3-oxo-piperazin-1-yl, 4-methylsulfonylpiperazin-1-yl, 3-azabicyclo[3.1.0]hexan-3-yl, azaspiro[2.4]heptan-S-yl, 2-azaspiro[4.4]nonan-2-yl, 6-azaspiro[3.4]octan-6-yl, S-azaspiro[2.4]heptan-5-yl, 7-azabicyclo[2.2.1]heptan-7-yl, 8-azabicyclo[3.2.1]octan-8-yl, 2-azabicyclo[2.2.2]octan-2-yl, and 3-azabicyclo[3.2.2]nonan-3-yl.

51. In embodiment 51, the compound is as provided in any one of embodiments 1 to 49, or a pharmaceutically acceptable salt thereof, wherein R⁴ and R⁵ together with the nitrogen atom to which they are attached form a ring selected from isoindolin-2-yl, 4-cyanoisoindolin-2-yl, 5-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, S-hydroxymethylisoindolin-2-yl, 4-difluoromethylisoindolin-2-yl, 5difluoromethylisoindolin-2-yl, 4-difluoromethoxyisoindolin-2-yl, 4-trifluoromethylisoindolin-2-yl, 5-trifluoromethylisoindolin-2-yl, 4-fluoroisoindolin-2-yl, 5-fluoroisoindolin-2-yl, 5,6-difluoroisoindolin-2-yl, 1-methylisoindoline-2-yl, 4-methylamino-carbonylisoindolin-2-yl, 4-dimethylaminocarbonyl-isoindolin-2-yl, 5-dimethylaminocarbonyl-isoindolin-2-yl, 4-(2-(dimethylamino)ethoxy)-isoindolin-2-yl, 4-(2-(methylamino)ethoxy)-isoindolin-2-yl, 4-dimethylaminomethylisoindolin-2-yl, 5-dimethylaminomethylisoindolin-2-yl, 4-methylaminomethylisoindolin-2-yl, 4-dimethyl-aminoisoindolin-2-yl, 4-methylaminoisoindolin-2-yl, 3-phenylisoindolin-2-yl, 4-phenylisoindolin-2-yl, 4-phenoxyisoindolin-2-yl, 1-benzylisoindolin-2-yl, 4-benzylisoindolin-2-yl, 4-(3-fluoro-phenoxy)isoindolin-2-yl, 4-(4-fluorophenoxy)isoindolin-2-yl, 4-(2-cyanophenoxy)isoindolin-2-yl, 4-(3-cyanophenoxy)-isoindolin-2-yl, 4-cyclohexylisoindolin-2-yl, 5-(hydroxymethyl)isoindolin-2-yl, 4-(2-hydroxyethoxy)isoindolin-2-yl, 4-(2-aminoethoxy)isoindolin-2-yl, 5-(cyanomethyl)-isoindolin-2-yl, 4-(cyanomethyl)isoindolin-2-yl, indolin-1-yl, 2-methylindolin-1-yl, 4-cyanoindolin-1-yl, 5-cyanoindolin-1-yl, 6-cyanoindolin-1-yl, 4-hydroxymethylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloroindolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxyindolin-1-yl, 6-trifluoromethoxylindolin-1-yl, 4-((dimethylamino)methyl)indolin-1-yl, 5-((dimethylamino)methyl)indolin-1-yl, 6-((dimethyl-amino)methyl)indolin-1-yl, 4-((methylamino)methyl)indolin-1-yl, 5-((methylamino)methyl)-indolin-1-yl, 6-((methylamino)-methyl)indolin-1-yl, 6-methylaminocarbonylindolin-1-yl, 6-aminocarbonyl-indolin-1-yl, 6-dimethylaminocarbonylindolin-1-yl, 4-(2-(methylamino)ethoxy)-indolin-1-yl, 6-(2-(dimethylamino)ethoxy) indolin-1-yl, 4-(hydroxymethyl)indolin-1-yl, 5-phenylindolin-1-yl, 6-(1-methyl-6-oxo-1,6-dihydropyridin-2-yl)indolin-1-yl, 4-(1-hydroxyethyl)-indolin-1-yl, 4-(2-hydroxypropan-2-yl)indolin-1-yl, 6-(2-hydroxypropan-2-yl)indolin-1-yl, 4-(cyanomethyl)indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, 6-chloroindolin-1-yl, 3,4-dihydroisoquinolin-2-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepine-2-yl, and 1-methyl-1,2,3,5-tetrahydro-4H-benzo[e][1,4]diazepin-4-yl, 2,3-dihydrobenzo[e][1,4]oxazepin-1(5H)-yl.

52. In embodiment 52, the compound is as provided in any one of embodiments 1 to 49 and 51, or a pharmaceutically acceptable salt thereof, wherein R⁴ and R⁵ together with the nitrogen atom to which they are attached form a ring selected from isoindolin-2-yl, 4-cyanoisoindolin-2-yl, 5-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, 5-hydroxymethylisoindolin-2-yl, 4-difluoromethylisoindolin-2-yl, 5difluoromethylisoindolin-2-yl, 4-difluoromethoxyisoindolin-2-yl, 4-trifluoromethylisoindolin-2-yl, 5-trifluoromethylisoindolin-2-yl, 4-fluoroisoindolin-2-yl, 5-fluoroisoindolin-2-yl, 5,6-difluoroisoindolin-2-yl, 1-methyl-isoindolin-2-yl, 4-methylamino-carbonylisoindolin-2-yl, 4-dimethylaminocarbonylisoindolin-2-yl, 5-dimethylaminocarbonyl-isoindolin-2-yl, 4-(2-(dimethylamino)ethoxy)isoindolin-2-yl, 4-(2-(methylamino)ethoxy)-isoindolin-2-yl, 4-dimethylaminomethylisoindolin-2-yl, 5-dimethylamino-methylisoindolin-2-yl, 4-methylaminomethylisoindolin-2-yl, 4-dimethylaminoisoindolin-2-yl, 4-methylaminoisoindolin-2-yl, 3-phenylisoindolin-2-yl, 4-phenylisoindolin-2-yl, 4-phenoxy-isoindolin-2-yl, 1-benzylisoindolin-2-yl, 4-benzylisoindolin-2-yl, 4-(3-fluorophenoxy)isoindolin-2-yl, 4-(4-fluorophenoxy)isoindolin-2-yl, 4-(2-cyanophenoxy)isoindolin-2-yl, 4-(3-cyano-phenoxy)isoindolin-2-yl, 4-cyclohexylisoindolin-2-yl, 5-(hydroxymethyl)isoindolin-2-yl, 4-(2-hydroxyethoxy)isoindolin-2-yl, 4-(2-aminoethoxy)isoindolin-2-yl, 5-(cyanomethyl)isoindolin-2-yl, and 4-(cyanomethyl)isoindolin-2-yl.

53. In embodiment 53, the compound is as provided in any one of embodiments 1 to 49, and 51, or a pharmaceutically acceptable salt thereof, wherein R⁴ and R⁵ together with the nitrogen atom to which they are attached form a ring selected from indolin-1-yl, 2-methylindolin-1-yl, 4-cyanoindolin-1-yl, 5-cyanoindolin-1-yl, 6-cyanoindolin-1-yl, 4-hydroxymethylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloro-indolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxyindolin-1-yl, 6-trifluoromethoxylindolin-1-yl, 4-((dimethylamino)methyl)indolin-1-yl, 5-((dimethyl-amino)methyl)indolin-1-yl, 6-((dimethyl-amino)methyl)indolin-1-yl, 4-((methylamino)methyl)-indolin-1-yl, 5-((methylamino)methyl)-indolin-1-yl, 6-((methylamino)-methyl)indolin-1-yl, 6-methylaminocarbonylindolin-1-yl, 6-aminocarbonyl-indolin-1-yl, 6-dimethylaminocarbonyl-indolin-1-yl, 4-(2-(methylamino)-ethoxy)indolin-1-yl, 6-(2-(dimethylamino)ethoxy) indolin-1-yl, 4-(hydroxymethyl)indolin-1-yl, 5-phenylindolin-1-yl, 6-(1-methyl-6-oxo-1,6-dihydropyridin-2-yl)indolin-1-yl, 4-(1-hydroxyethyl)indolin-1-yl, 4-(2-hydroxypropan-2-yl)indolin-1-yl, 6-(2-hydroxypropan-2-yl)indolin-1-yl, 4-(cyanomethyl)indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, and 6-chloroindolin-1-yl.

54. In embodiment 54, the compound is as provided in any one of embodiments 1 to 49, and 51, or a pharmaceutically acceptable salt thereof, wherein R⁴ and R⁵ together with the nitrogen atom to which they are attached form a ring selected from 3,4-dihydroisoquinolin-2(1H)-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepin-2-yl, 1-methyl-1,2,3,5-tetrahydro-4H-benzo[e][1,4]diazepin-4-yl, and 2,3-dihydrobenzo[e][1,4]oxazepin-1(5H)-yl.

55. In embodiment 55, the compound is as provided in any one of embodiments 1 to 24, or a pharmaceutically acceptable salt thereof, wherein R⁴ and R⁵ are independently hydrogen, alkyl, haloalkyl, alkoxyalkyl, hydroxalkyl, aminoalkyl, cycloalkyl, cycloalkylalkyl, fused cycloalkyl, aryl, aralkyl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein each of the forementioned ring, either alone or as part of another group, is substituted with R^{p}, R^{q}, and R^{r}.

56. In embodiment 56, the compound is as provided in any one of embodiments 1 to 24 and 55, or a pharmaceutically acceptable salt thereof, wherein R⁴ is hydrogen, alkyl, haloalkyl, alkoxyalkyl, hydroxalkyl, aminoalkyl, cycloalkyl, cycloalkylalkyl, fused cycloalkyl, aryl, aralkyl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, and spiro heterocyclylalkyl and R⁵ is alkyl, haloalkyl, alkoxyalkyl, hydroxalkyl, aminoalkyl, cycloalkyl, cycloalkylalkyl, fused cycloalkyl, aryl, aralkyl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, or spiro heterocyclylalkyl, wherein each of the forementioned ring, either alone or as part of another group, is substituted with R^{p}, R^{q}, and R^{r}.

57. In embodiment 57, the compound is as provided in any one of embodiments 1 to 56, or a pharmaceutically acceptable salt thereof, wherein W is N.

58. In embodiment 58, the compound is as provided in any one of embodiments 1 to 56, or a pharmaceutically acceptable salt thereof, wherein W is CH or C (when attached to R⁶).

59. In embodiment 59, the compound is as provided in any one of embodiments 1 to 58, or a pharmaceutically acceptable salt thereof, wherein R⁶ is hydrogen, fluoro, methyl, cyclopropyl, cyano, difluoromethyl, or trifluoromethyl.

59a. In embodiment 59a, the compound is as provided in any one of embodiments 1 to 58, or a pharmaceutically acceptable salt thereof, wherein R⁶ is hydrogen, fluoro, cyano, difluoromethyl, or trifluoromethyl.

59b. In embodiment 59b, the compound is as provided in any one of embodiments 1 to 58, or a pharmaceutically acceptable salt thereof, wherein R⁶ is hydrogen.

60. In embodiment 60, the compound is as provided in any one of embodiments 1, 2 and 10 to 59b, or a pharmaceutically acceptable salt thereof, wherein R^{8A} is hydrogen.

61. In embodiment 61, the compound is as provided in any one of embodiments 1, 2 and 10 to 59b, or a pharmaceutically acceptable salt thereof, wherein R^{8A} is alkyl.

62. In embodiment 62, the compound is as provided in any one of embodiments 1, 2, 10 to 59b, and 61, or a pharmaceutically acceptable salt thereof, wherein R^{8A} is methyl, ethyl, or propyl.

63. In embodiment 63, the compound is as provided in any one of embodiments 1, 2, 8, 9, and 11 to 62, or a pharmaceutically acceptable salt thereof, wherein R⁸ is hydrogen, methyl, ethyl, propyl, fluoro, hydroxymethyl, or cyano.

64. In embodiment 58, the compound is as provided in any one of embodiments 1, 2, 9, and 11 to 59b, or a pharmaceutically acceptable salt thereof, wherein R⁸ is hydrogen, fluoro, or cyano.

65. In embodiment 65, the compound is as provided in any one of embodiments 1 to 64, or a pharmaceutically acceptable salt thereof, wherein R⁹ and R¹⁰ are independently alkyl, cycloalkyl, bridged cycloalkyl, spiro cycloalkyl, heterocyclyl, bridged heterocyclyl, or spiro heterocyclyl, wherein:
(A) cycloalkyl, bridged cycloalkyl, and spiro cycloalkyl of R⁹ and R¹⁰ are substituted with one or two R^{a}; and
(B) heterocyclyl, bridged heterocyclyl, and spiro heterocyclyl of R⁹ and R¹⁰ are substituted with R^{b}, R^{c}, and R^{d}.

66. In embodiment 66, the compound is as provided in any one of embodiments 1 to 65, or a pharmaceutically acceptable salt thereof, wherein R⁹ and R¹⁰ are alkyl.

67. In embodiment 67, the compound is as provided in any one of embodiments 1 to 66, or a pharmaceutically acceptable salt thereof, wherein R⁹ and R¹⁰ are methyl, ethyl, propyl, or butyl.

68. In embodiment 68, the compound is as provided in any one of embodiments 1 to 67, or a pharmaceutically acceptable salt thereof, wherein R⁹ and R¹⁰ are methyl or ethyl.

69. In embodiment 69, the compound is as provided in any one of embodiments 1 to 67, or a pharmaceutically acceptable salt thereof, wherein R⁹ and R¹⁰ are propyl or butyl.

70. In embodiment 70, the compound is as provided in any one of embodiments 1 to 67 and 69, or a pharmaceutically acceptable salt thereof, wherein R⁹ and R¹⁰ are isopropyl, iso-butyl, sec-butyl, or tert-butyl.

71. In embodiment 71, the compound is as provided in any one of embodiments 1 to 67 and 69, or a pharmaceutically acceptable salt thereof, wherein R⁹ and R¹⁰ are isopropyl or tert-butyl.

72. In embodiment 72, the compound is as provided in any one of embodiments 1 to 65, or a pharmaceutically acceptable salt thereof, wherein R⁹ and R¹⁰ are independently cycloalkyl, bridged cycloalkyl, or spiro cycloalkyl wherein the cycloalkyl, bridged cycloalkyl, and spiro cycloalkyl of R⁹ and R¹⁰ are substituted with one or two R^{a}.

73. In embodiment 73, the compound is as provided in any one of embodiments 1 to 65 and 72, or a pharmaceutically acceptable salt thereof, wherein R⁹ and R¹⁰ are independently cycloalkyl, bridged cycloalkyl, or spiro cycloalkyl wherein the cycloalkyl, bridged cycloalkyl, and spiro cycloalkyl of R⁹ and R¹⁰ are substituted with one R^{a}.

74. In embodiment 74, the compound is as provided in any one of embodiments 1 to 65, 72 and 73, or a pharmaceutically acceptable salt thereof, wherein the one or two R^{a} are independently selected from hydrogen, deuterium, alkyl, deuteroalkyl, halo, haloalkyl, and cyano.

75. In embodiment 75, the compound is as provided in any one of embodiments 1 to 65 and 72 to 74, or a pharmaceutically acceptable salt thereof, wherein the one or two R^{a} are independently selected from hydrogen, deuterium, methyl, trideuteromethyl, cyclopropyl, fluoro, chloro, difluoromethyl, trifluoromethyl, hydroxymethyl, cyano, amino, methylamino, diethylamino, and dimethylamino.

76. In embodiment 76, the compound is as provided in of any one of embodiments 1 to 65 and 72 to 75, or a pharmaceutically acceptable salt thereof, wherein the one or two R^{a} are independently selected from hydrogen, deuterium, methyl, trideuteromethyl, fluoro, chloro, difluoromethyl, trifluoromethyl, and cyano.

77. In embodiment 77, the compound is as provided in any one of embodiments 1 to 65 and 72 to 76, or a pharmaceutically acceptable salt thereof, wherein R⁹ and R¹⁰ are independently cycloalkyl and bridged cycloalkyl, each ring is substituted with R^{a} as defined therein.

78. In embodiment 78, the compound is as provided in any one of embodiments 1 to 65 and 72 to 76, or a pharmaceutically acceptable salt thereof, wherein R⁹ and R¹⁰ are spiro cycloalkyl substituted with R^{a} as defined therein.

79. In embodiment 79, the compound is as provided in any one of embodiments 1 to 65 and 72 to 77, or a pharmaceutically acceptable salt thereof, wherein R⁹ and R¹⁰ are cycloalkyl substituted with R^{a} as defined therein.

80. In embodiment 80, the compound is as provided in any one of embodiments 1 to 65 and 72 to 77, or a pharmaceutically acceptable salt thereof, wherein R⁹ and R¹⁰ are bridged cycloalkyl substituted with R^{a} as defined therein.

81. In embodiment 81, the compound is as provided in any one of embodiments 1 to 65, or a pharmaceutically acceptable salt thereof, wherein R⁹ and R¹⁰ are independently heterocyclyl, bridged heterocyclyl, or spiro heterocyclyl wherein the heterocyclyl, bridged heterocyclyl, and spiro heterocyclyl of R⁹ and R¹⁰ are substituted with R^{b}, R^{c}, and R^{d}.

82. In embodiment 82, the compound is as provided in any one of embodiments 1 to 65 and 81, or a pharmaceutically acceptable salt thereof, wherein R⁹ and R¹⁰ are independently heterocyclyl or bridged heterocyclyl, each ring is substituted with R^{b}, R^{c}, and R^{d}.

83. In embodiment 83, the compound is as provided in any one of embodiments 1 to 65 and 81, or a pharmaceutically acceptable salt thereof, wherein R⁹ and R¹⁰ are spiro heterocyclyl substituted with R^{b}, R^{c}, and R^{d}.

84. In embodiment 84, the compound is as provided in any one of embodiments 1 to 65, 81 and 82, or a pharmaceutically acceptable salt thereof, wherein R⁹ and R¹⁰ are heterocyclyl substituted with R^{b}, R^{c}, and R^{d}.

85. In embodiment 85, the compound is as provided in any one of embodiments 1 to 65, 81, and 82, or a pharmaceutically acceptable salt thereof, wherein R⁹ and R¹⁰ are bridged heterocyclyl substituted with R^{b}, R^{c}, and R^{d}.

86. In embodiment 86, the compound is as provided in any one of embodiments 1 to 65, 72 to 77, 79 to 82, 84, and 85, or a pharmaceutically acceptable salt thereof, wherein the cycloalkyl and bridged cycloalkyl of R⁹ and R¹⁰ are selected from:

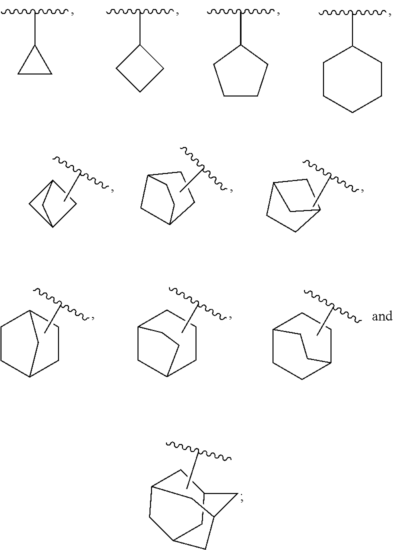

and the heterocyclyl and bridged heterocyclyl of R⁹ and R¹⁰ are selected from:

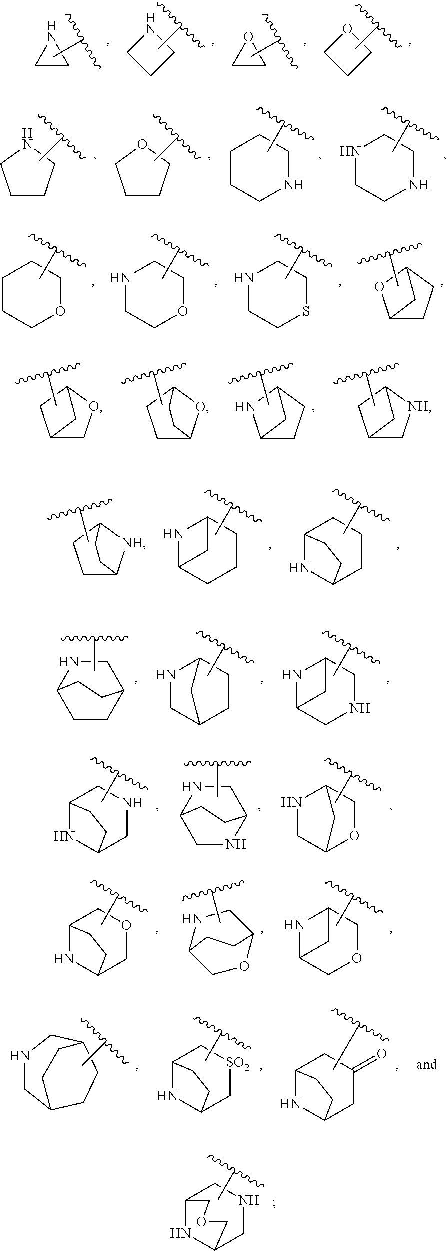

wherein each cycloalkyl and bridged cycloalkyl is substituted with R^{a} as defined therein; and each heterocyclyl and bridged heterocyclyl is substituted with R^{b}, R^{c}, and R^{d}.

87. In embodiment 87, the compound is as provided in any one of embodiments 1 to 65, 72 to 77, 79 to 82, and 84 to 85, or a pharmaceutically acceptable salt thereof, wherein the cycloalkyl and bridged cycloalkyl of R⁹ and R¹⁰ are selected from:

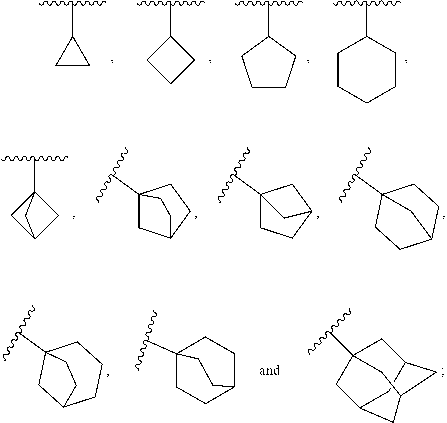

and the heterocyclyl and bridged heterocyclyl of R⁹ and R¹⁰ are selected from:

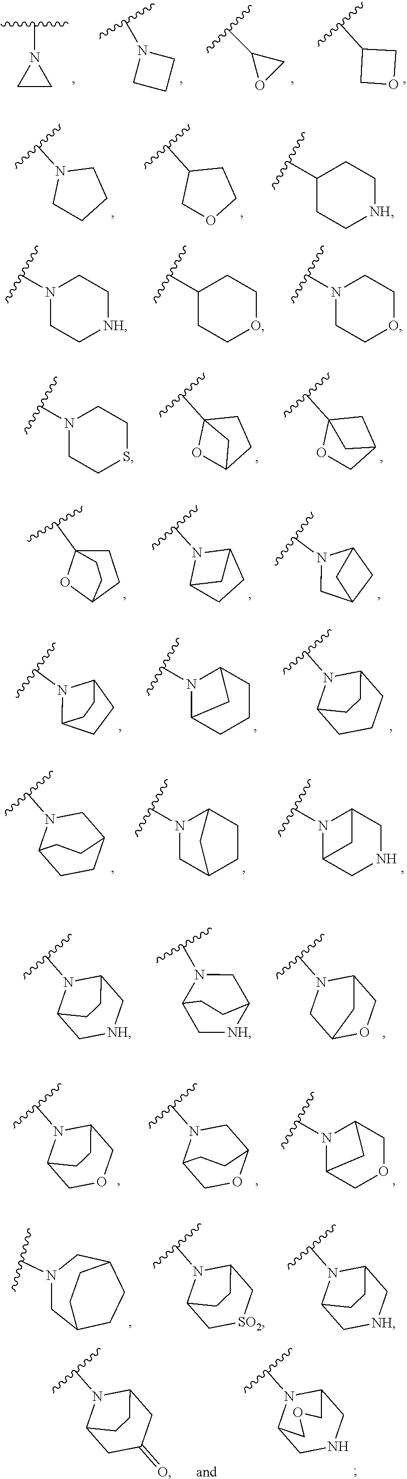

wherein each cycloalkyl and bridged cycloalkyl is substituted with R^{a} as defined therein; and heterocyclyl and bridged heterocyclyl is substituted with R^{b}, R^{c}, and R^{d} as defined therein.

88. In embodiment 88, the compound is as provided in any one of embodiments 1 to 65, 72 to 77, 79, and 80, or a pharmaceutically acceptable salt thereof, wherein the cycloalkyl and bridged cycloalkyl of R⁹ and R¹⁰ are selected from:

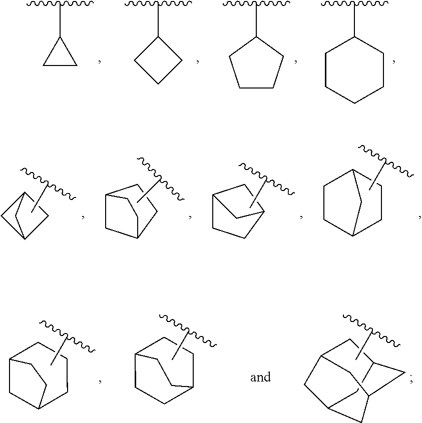

wherein each cycloalkyl and bridged cycloalkyl is substituted with R^{a} as defined therein.

89. In embodiment 89, the compound is as provided in any one of embodiments 1 to 65, 72 to 77, 79, and 80, or a pharmaceutically acceptable salt thereof, wherein the cycloalkyl and bridged cycloalkyl of R⁹ and R¹⁰, are selected from:

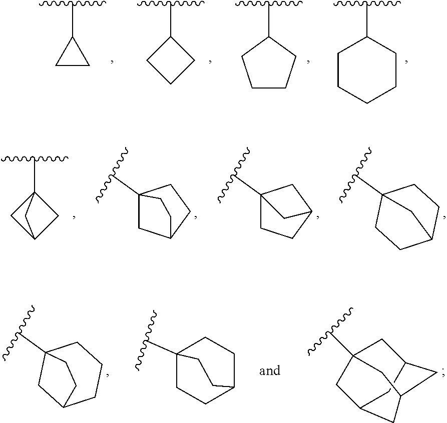

wherein each cycloalkyl and bridged cycloalkyl is substituted with R^{a} as defined therein.

90. In embodiment 90, the compound is as provided in any one of embodiments 1 to 65, 74 to 76, 81, 82, 84, and 85, or a pharmaceutically acceptable salt thereof, wherein the heterocyclyl and bridged heterocyclyl of R⁹ and R¹⁰ are selected from:

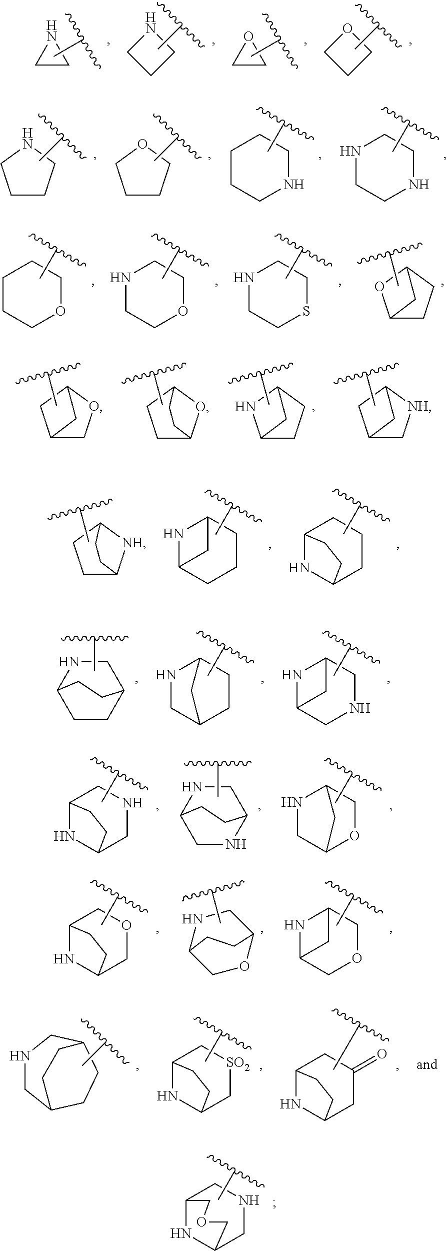

wherein each ring is substituted with R^{b}, R^{c}, and R^{d} as defined therein.

91. In embodiment 91, the compound is as provided in any one of embodiments 1 to 65, 74 to 76, 81, 82, 84, and 85, or a pharmaceutically acceptable salt thereof, wherein the heterocyclyl and bridged heterocyclyl of R⁹ and R¹⁰ are selected from:

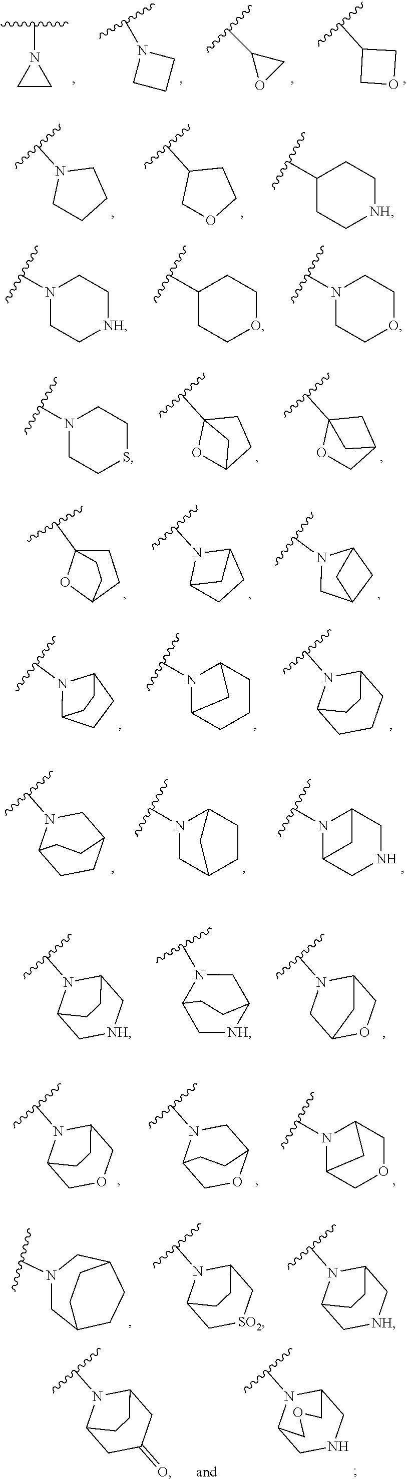

each ring substituted with R^{b}, R^{c}, and R^{d} as defined therein.

92. In embodiment 92, the compound is as provided in any one of embodiments 1 to 65, 72 to 77, 79, 86, and 87, or a pharmaceutically acceptable salt thereof, wherein the cycloalkyl of ring R⁹ and R¹⁰ are selected from:

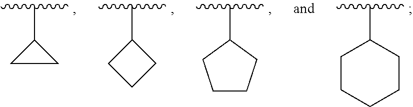

wherein each cycloalkyl is substituted with R^{a} as defined therein.

93. In embodiment 93, the compound is as provided in any one of embodiments 1 to 65, 72 to 77, 79, 86, 87, and 92, or a pharmaceutically acceptable salt thereof, wherein the cycloalkyl of ring R⁹ and R¹⁰ are selected from:

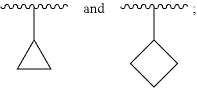

wherein each cycloalkyl is substituted with one or two R^{a} as defined therein.

94. In embodiment 94, the compound is as provided in any one of embodiments 1 to 65, 72 to 77, and 80, or a pharmaceutically acceptable salt thereof, wherein the bridged cycloalkyl of R⁹ and R¹⁰ are selected from:

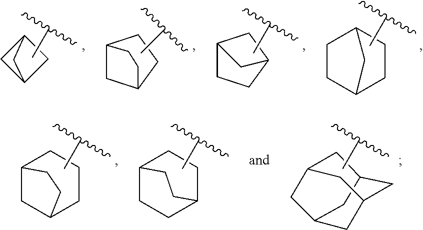

wherein each bridged cycloalkyl is substituted with R^{a} as defined therein.

95. In embodiment 95, the compound is as provided in any one of embodiments 1 to 65, 72 to 77, and 80, or a pharmaceutically acceptable salt thereof, wherein the bridged cycloalkyl of R⁹ and R¹⁰ are selected from:
selected from:

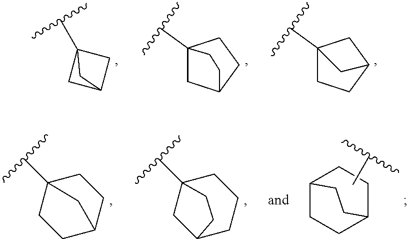

wherein each bridged cycloalkyl is substituted with R^{a} as defined therein.

96. In embodiment 96, the compound is as provided in any one of embodiments 1 to 65, 72 to 77, 80, and 95, or a pharmaceutically acceptable salt thereof, wherein the bridged cycloalkyl of R⁹ and R¹⁰ are:

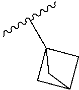 [SMILES: *C12CC(C1)C2]

and is substituted with R^{a} as defined therein.

97. In embodiment 97, the compound is as provided in any one of embodiments 1 to 65, 74 to 76, 81, 82, 84, and 92 to 96, or a pharmaceutically acceptable salt thereof, wherein the heterocyclyl of R⁹ and R¹⁰ is selected from:

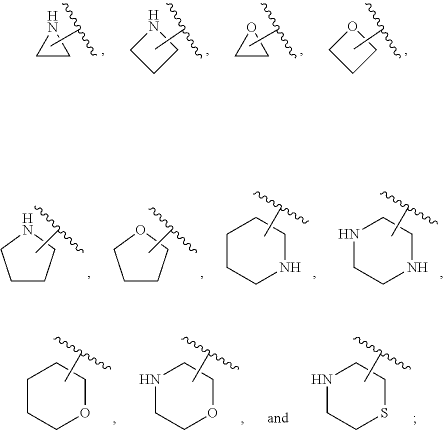

wherein each heterocyclyl is substituted with R^{b}, R^{c}, and R^{d} as defined therein.

98. In embodiment 98, the compound is as provided in any one of embodiments 1 to 65, 74 to 76, 81, 82, 84, and 92 to 97, or a pharmaceutically acceptable salt thereof, wherein the heterocyclyl of R⁹ and R¹⁰ is selected from:

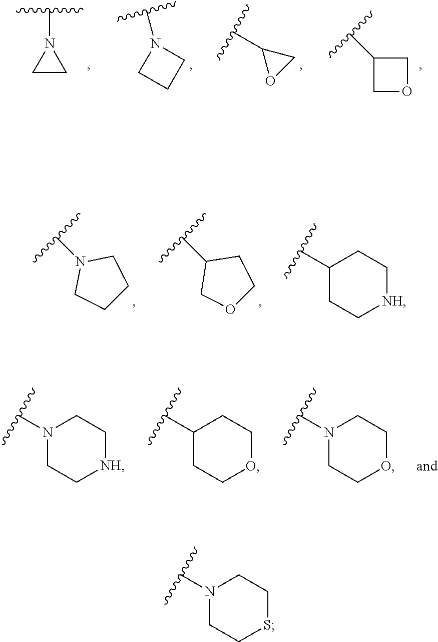

wherein each heterocyclyl is substituted with R^{b}, R^{c}, and R^{d} as defined therein.

99. In embodiment 99, the compound is as provided in any one of embodiments 1 to 65, 74 to 76, 81, 82, 84, 92 to 96, and 98, or a pharmaceutically acceptable salt thereof, wherein the heterocyclyl of R⁹ and R¹⁰ is selected from:

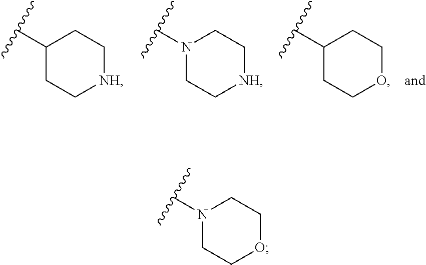 [SMILES: *C1CCNCC1 ; *N1CCNCC1 ; *C1CCOCC1 ; "and" ; *N1CCOCC1]

wherein each heterocyclyl is substituted with R^{b}, R^{c}, and R^{d} as defined therein.

100. In embodiment 100, the compound is as provided in any one of embodiments 1 to 65, 74 to 76, 81, 82, 85, and 92 to 96, or a pharmaceutically acceptable salt thereof, wherein the bridged heterocyclyl of R⁹ and R¹⁰ is selected from:

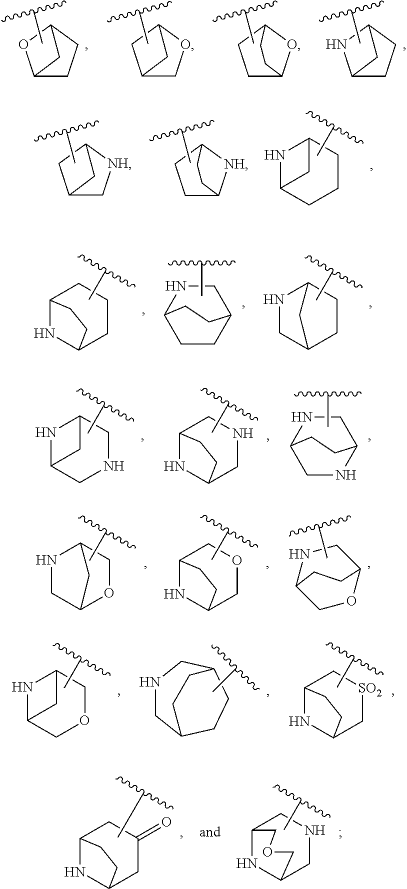

wherein each bridged heterocyclyl is substituted with R^{b}, R^{c}, and R^{d}.

101. In embodiment 101, the compound is as provided in any one of embodiments 1 to 65, 74 to 76, 81, 82, 85, and 92 to 96, or a pharmaceutically acceptable salt thereof, wherein the bridged heterocyclyl of R⁹ and R¹⁰ is selected from:

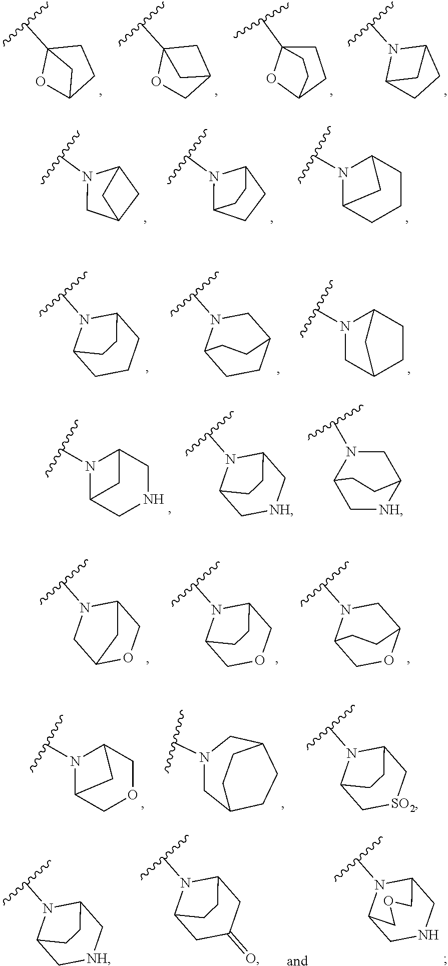

wherein each bridged heterocyclyl is substituted with R^{b}, R^{c}, and R^{d} as defined therein

102. In embodiment 102, the compound is as provided in any one of embodiments 1 to 65, 74 to 76, 81, 82, 85, 92 to 96, and 101, or a pharmaceutically acceptable salt thereof, wherein the bridged heterocyclyl of R⁹ and R¹⁰ is selected from:

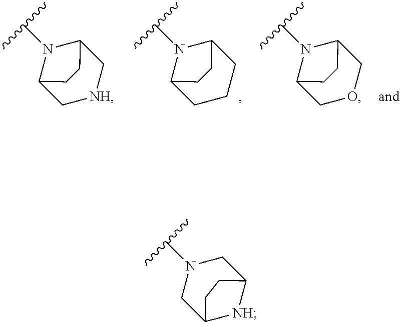 [SMILES: *N1C2CCC1CNC2 ; *N1C2CCCC1CC2 ; *N1C2CCC1COC2 ; "and" ; *N1CC2CCC(C1)N2]

wherein each bridged heterocyclyl is substituted with R^{b}, R^{c}, and R^{d} as defined therein.

103. In embodiment 103, the compound is as provided in any one of embodiments 1 to 65, 72 to 80, and 86 to 102, or a pharmaceutically acceptable salt thereof, wherein R^{a} is hydrogen, deuterium, chloro, fluoro, difluoromethyl, or trifluoromethyl.

104. In embodiment 104, the compound is as provided in any one of embodiments 1 to 65, 72 to 80, and 86 to 103, or a pharmaceutically acceptable salt thereof, wherein R^{a} is hydrogen.

105. In embodiment 105, the compound is as provided in any one of embodiments 1 to 65, 72 to 80, and 86 to 103, or a pharmaceutically acceptable salt thereof, wherein R^{a} is fluoro or chloro.

106. In embodiment 106, the compound is as provided in any one of embodiments 1 to 65, 72 to 80, and 86 to 103, or a pharmaceutically acceptable salt thereof, wherein R^{a} is difluoromethyl, or trifluoromethyl.

107. In embodiment 107, the compound is as provided in any one of embodiments 1 to 65, 72 to 74, 77 to 80, and 86 to 102, or a pharmaceutically acceptable salt thereof, wherein R^{a} is hydrogen or haloalkyl.

108. In embodiment 108, the compound is as provided in any one of embodiments 1 to 65, 72 to 74, 77 to 80, and 86 to 102, or a pharmaceutically acceptable salt thereof, wherein R^{a} is hydrogen or halo.

109. In embodiment 109, the compound is as provided in any one of embodiments 1 to 65, 72, 73, 77 to 80, and 86 to 102, or a pharmaceutically acceptable salt thereof, wherein R^{a} is halo or haloalkyl.

110. In embodiment 110, the compound is as provided in any one of embodiments 1 to 65, 72 to 77, 79, 80, 86, 87, 88, 90, 91, and 97 to 102, or a pharmaceutically acceptable salt thereof, wherein the bridged cycloalkyl of R⁹ and R¹⁰ is bicyclo[1.1.1]pentan-1-yl, 3-fluorobicyclo[1.1.1]pentan-1-yl, 3-chlorobicyclo-[1.1.1]pentan-1-yl, 3-(hydroxymethyl)-bicyclo[1.1.1]pentan-1-yl, 3-(trifluoromethyl)-bicyclo[1.1.1]pentan-1-yl, 3-(difluoromethyl)-bicyclo[1.1.1]pentan-1-yl, and cycloalkyl of R⁹ and R¹⁰ is cyclobutyl, cyclopropyl, 1-(difluoromethyl)cyclobutyl, 1-(trifluoromethyl)cyclobutyl, 1-(difluoromethyl)cyclopropyl, or 1-(trifluoromethyl)cyclopropyl.

111. In embodiment 111, the compound is as provided in any one of embodiments 1 to 65, 72, 73, 77, 80, 86, 87, 88, 90, 91, 97 to 102, and 110, or a pharmaceutically acceptable salt thereof, wherein the bridged cycloalkyl of R⁹ and R¹⁰ is bicyclo[1.1.1]pentan-1-yl, 3-fluorobicyclo[1.1.1]pentan-1-yl, 3-chlorobicyclo[1.1.1]pentan-1-yl, 3-(hydroxymethyl)-bicyclo[1.1.1]pentan-1-yl, 3-(trifluoromethyl)bicyclo[1.1.1]pentan-1-yl, or 3-(difluoromethyl)-bicyclo[1.1.1]pentan-1-yl.

112. In embodiment 112, the compound is as provided in any one of embodiments 1 to 65, 72, 73, 79, 86, 87, 88, 90, 91, 97 to 102, and 110, or a pharmaceutically acceptable salt thereof, wherein the cycloalkyl of R⁹ and R¹⁰ is cyclobutyl, cyclopropyl, 1-(difluoromethyl)cyclobutyl, 1-(trifluoromethyl)-cyclobutyl, 1-(difluoromethyl)-cyclopropyl, or 1-(trifluoromethyl)cyclopropyl.

113. In embodiment 113, the compound is as provided in any one of embodiments 1 to 65, 72, 73, 79, 86, 87, 88, 90, 91, 97 to 102, 110, and 112, or a pharmaceutically acceptable salt thereof, wherein cycloalkyl of R⁹ and R¹⁰ is 1-(difluoromethyl)cyclobutyl, 1-(trifluoromethyl)cyclobutyl, 1-(difluoromethyl)-cyclopropyl, or 1-(trifluoromethyl)cyclopropyl.

114a. In embodiment 114a, the compound is as provided in any one of embodiments 1 to 65, 74 to 76, 81 to 113, or a pharmaceutically acceptable salt thereof, wherein R^{b} and R^{c} are independently selected from hydrogen, alkyl, halo, haloalkyl, hydroxy, and cyano and R^{d} is selected from hydrogen, alkyl, deuteroalkyl, cycloalkyl (as defined in the first aspect), haloalkyl, amino, alkylamino, dialkylamino, heterocyclyl, phenyl, phenylalkyl, and heteroaryl wherein phenyl, phenylalkyl, heteroaryl, and heterocyclyl are substituted with Rₑ, R_{f}, and R^{g}.

114. In embodiment 114, the compound is as provided in any one of embodiments 1 to 65, 74 to 76, 81 to 113, or a pharmaceutically acceptable salt thereof, wherein R^{b} and R^{c} are independently selected from hydrogen, methyl, fluoro, chloro, difluoromethyl, trifluoromethyl, 2,2-difluoroethyl, 2,2,2-trifluoroethyl, 3,3,3-trifluoropropyl, hydroxy, and cyano, and R^{d} is selected from hydrogen, methyl, trideuteromethyl, cyclopropyl, cyclobutyl, cyclopentyl, cyclohexyl, 2,2-difluoroethyl, 2,2,2-trifluoroethyl, 3,3,3-trifluoropropyl, amino, dimethylamino, diethylamino, 3,3-difluorocyclobutyl, 4,4-difluorocyclohexyl, 3-hydroxy-3-methylcyclobutyl, 3-cyano-3-methylcyclobutyl, oxetan-3-yl, tetrahydrofuran-2-yl, tetrahydropyran-4-yl, 1,1-dioxidothietan-3-yl, 1,1-dioxidotetrahydro-2H-thiopyran-4-yl, benzyl, phenyl, pyridin-2-yl, pyridin-3-yl, and pyridin-4-yl.

115. In embodiment 115, the compound is as provided in any one of embodiments 1 to 65, 74 to 76, 81 to 113, and 114, or a pharmaceutically acceptable salt thereof, wherein R^{b} is hydrogen, R^{c} is selected from hydrogen, methyl, fluoro, chloro, difluoromethyl, trifluoromethyl, 2,2-difluoroethyl, 2,2,2-trifluoroethyl, 3,3,3-trifluoropropyl, hydroxy, and cyano, and R^{d} is selected from hydrogen, methyl, trideuteromethyl, cyclopropyl, cyclobutyl, cyclopentyl, cyclohexyl, 2,2-difluoroethyl, 2,2,2-trifluoroethyl, 3,3,3-trifluoropropyl, amino, dimethylamino, diethylamino, 3,3-difluorocyclobutyl, 4,4-difluorocyclohexyl, 3-hydroxy-3-methylcyclobutyl, 3-cyano-3-methylcyclobutyl, oxetan-3-yl, tetrahydrofuran-2-yl, tetrahydropyran-4-yl, 1,1-dioxidothietan-3-yl, 1,1-dioxidotetrahydro-2H-thiopyran-4-yl, benzyl, phenyl, pyridin-2-yl, pyridin-3-yl, and pyridin-4-yl.

116. In embodiment 116, the compound is as provided in any one of embodiments 1 to 65, 74 to 76, 81, 82, 84 to 90 to 97, 100, and 103 to 113, or a pharmaceutically acceptable salt thereof, wherein (1) the heterocyclyl of R⁹ and R¹⁰ is 3,5-dimethylmorpholino, 4-methyltetrahydro-2H-pyran-4-yl, 2,2,4-trimethylpiperazin-1-yl, 4-methylpiperidin-4-yl, 1,4-dimethylpiperidin-4-yl, 3-oxo-8-azabicyclo[3.2.1]octan-8-yl, 2,2-dimethylpyrrolidin-1-yl, 2,2-dimethylazetidin-1-yl, 2,4-dimethylazetidin-1-yl, 2,4,6-trimethylpiperazin-1-yl, 2,6-dimethylpiperazin-1-yl, or 2,5-dimethylpyrrolidin-1-yl; and
(ii) the bridged heterocyclyl of R⁹ and R¹⁰ is 2-oxabicyclo[2.1.1]hexan-1-yl, 7-azabicyclo[2.2.1]heptan-7-yl, 3-oxa-8-azabicyclo[3.2.1]octan-8-yl, 3-methyl-3,8-diazabicyclo[3.2.1]octan-8-yl, 3,3-difluoro-8-azabicyclo[3.2.1]octan-8-yl, 3-(methyl-d3)-3,8-diazabicyclo[3.2.1]octan-8-yl, 3-cyclopropyl-3,8-diazabicyclo[3.2.1]octan-8-yl, 3-(2,2,2-trifluoroethyl)-3,8-diazabicyclo[3.2.1]octan-8-yl, 3-(2,2-difluoroethyl)-3,8-diazabicyclo[3.2.1]-octan-8-yl, 3,8-diazabicyclo[3.2.1]octan-8-yl, 2-azabicyclo[2.2.2]octan-2-yl, 8-azabicyclo[3.2.1]octan-8-yl, 3-(3,3,3-trifluoropropyl)-3,8-diazabicyclo[3.2.1]octan-8-yl, 3-azabicyclo[3.2.2]nonan-3-yl, 3-methyl-3,6-diazabicyclo[3.1.1]heptan-6-yl, 8-methyl-3,8-diazabicyclo[3.2.1]octan-3-yl, 3-(dimethylamino)-8-azabicyclo[3.2.1]octan-8-yl, 3-(3,3-difluorocyclobutyl)-3,8-diazabicyclo[3.2.1]octan-8-yl, 3-(oxetan-3-yl)-3,8-diazabicyclo-[3.2.1]octan-8-yl, 5-methyl-2,5-diazabicyclo[2.2.2]octan-2-yl, 3-hydroxy-3-methyl-8-azabicyclo[3.2.1]octan-8-yl, 3-cyano-3-methyl-8-azabicyclo[3.2.1]octan-8-yl, 3-(4,4-difluoro-cyclohexyl)-3,8-diazabicyclo[3.2.1]octan-8-yl, 3-(tetrahydro-2H-pyran-4-yl)-3,8-diazabicyclo-[3.2.1]octan-8-yl, 8~ (pyridin-2-yl)-3,8-diazabicyclo[3.2.1]octan-3-yl, 3-(1,1-dioxidotetrahydro-2H-thiopyran-4-yl)-3,8-diazabicyclo[3.2.1]octan-8-yl, 3-benzyl-3,8-diazabicyclo[3.2.1]octan-8-yl, 3-phenyl-3,8-diazabicyclo[3.2.1]octan-8-yl, 3-(pyridin-2-yl)-3,8-diazabicyclo[3.2.1]octan-8-yl, 3-(tetrahydrofuran-3-yl)-3,8-diazabicyclo[3.2.1]octan-8-yl, 3-(1,1-dioxidothietan-3-yl)-3,8-diazabicyclo[3.2.1]octan-8-yl, 3-(3-hydroxy-3-methylcyclobutyl)-3,8-diazabicyclo[3.2.1]octan-8-yl, 3,3-dioxido-3-thia-8-azabicyclo[3.2.1]octan-8-yl, 3-(3-cyano-3-methylcyclobutyl)-3,8-diazabicyclo[3.2.1]octan-8-yl, 3-cyclobutyl-3,8-diazabicyclo[3.2.1]octan-8-yl, 2-(1-cyano-7-azabicyclo[2.2.1]heptan-7-yl, 4-(3,3-difluorocyclobutyl)-2,6-dimethylpiperazin-1-yl, 7-methyl-3-oxa-7,9-diazabicyclo[3.3.1]nonan-9-yl, or 3-cyclopropyl-3,8-diazabicyclo[3.2.1]octan-8-yl.

117. In embodiment 117, the compound is as provided in any one of embodiments 1 to 65, 74 to 76, 81, 82, 85 to 90, 92 to 100, 103 to 113 and 117, or a pharmaceutically acceptable salt thereof, wherein bridged heterocyclyl of R⁹ and R¹⁰ is 3-(3,3-difluorocyclobutyl)-3,8-diazabicyclo[3.2.1]octan-8-yl.

118. In embodiment 118, the compound of Formula (I) is as provided in any one of embodiments 1 to 65, 74 to 76, 81, 82, 85 to 90, 92 to 100, and 103 to 113, or a pharmaceutically acceptable salt thereof, wherein bridged heterocyclyl of R⁹ and R¹⁰ is 3-methyl-3,8-diazabicyclo[3.2.1]octan-8-yl.

119. In embodiment 119, the compound is as provided in any one of embodiments 1 to 65, or a pharmaceutically acceptable salt thereof, wherein R⁹ and R¹⁰ are independently hydrogen, hydroxyalkyl, alkoxyalkyl, cyanoalkyl, alkylsulfonyl, alkylsulfonylalkyl, amino, alkylamino, dialkylamino, substituted amino, aminoalkyl, bicyclic cycloalkyl, bicyclic heterocyclyl, aryl, aralkyl, heteroaryl, or heteroaralkyl, wherein bicyclic cycloalkyl of R⁹ and R¹⁰ are substituted with one or two R^{a}; bicyclic heterocyclyl of R⁹ and R¹⁰ are substituted with R^{b}, R^{c}, and R^{d} and aryl, by itself or as part of aralkyl and heteroaryl, by itself or as part of heteroaralkyl, of R⁹ and R¹⁰ are substituted with R^{h}, R^{j}, and R^{k}.

120. In embodiment 120, the compound is as provided in any one of embodiments 1 to 65 and 119, or a pharmaceutically acceptable salt thereof, wherein R⁹ and R¹⁰ are haloalkyl, hydroxyalkyl, alkoxyalkyl, cyanoalkyl, phenyl, or heteroaryl wherein phenyl and heteroaryl of R⁹ and R¹⁰ are substituted with R^{h}, R^{j}, and R^{k}.

121. In embodiment 121, the compound is as provided in any one of embodiments 1 to 65, 119, and 120, or a pharmaceutically acceptable salt thereof, wherein R⁹ and R¹⁰ are 3-hydroxy-1,1-dimethylethyl, difluoromethyl, 1-fluoro-1-methylethyl, 1-methoxy-1-methylethyl, 1-cyano-1-methylethyl, 1-cyano-1-methylethyl, 2-cyano-1,1-dimethylethyl, 1-fluoro-1-methylethyl, 2,6-difluorophenyl, 2,6-dichlorophenyl, 2-methylphenyl, 2-chloro-6-fluorophenyl, 2-chlorophenyl, 2-fluorophenyl, 2-methylphenyl, 2-fluoro-6-methylphenyl, pyridin-4-yl, 1,1-dimethyl-2methylsulfonylethyl, 1-methyl-1-pyridin-3-ylethyl, N-methyl-N-phenylamino, N-methyl-N-(2-fluorophenyl)amino, N,N-bis cyclopropylamino, or bicyclo[3.1.0]hexyl.

122. In embodiment 122, the compound is as provided in any one of embodiments 1 to 121, or a pharmaceutically acceptable salt thereof, wherein R⁷ is -Q-(alk¹)ₙ₁-SO₂R¹¹, -Q¹-(alk²)ₙ₂-SO(═NR¹²) R¹³, -Q²-(alk³)ⁿ³-SO₂NR¹⁴R¹⁵, -Q³-(alk⁴)ₙ₄-COR¹⁶, -Q⁴-(alk⁵)ⁿ⁵-CONR¹⁷R¹⁸, -Q⁵-(alk⁶)ₙ₆-NR¹⁹COR²⁰, -Q⁶-(alk⁷)ₙ₇-NR²¹SO₂R²², heterocyclyl, bicyclic heterocyclyl, fused heterocyclyl, spiro heterocyclyl, bridged heterocyclyl, cyclylsulfoximinyl, bicyclylsulfoximinyl, spiro sulfoximinyl, aryl, or heteroaryl, wherein heterocyclyl, bicyclic heterocyclyl, fused heterocyclyl, spiro heterocyclyl, bridged heterocyclyl, cyclylsulfoximinyl, bicyclylsulfoximinyl, spiro sulfoximinyl, aryl, and heteroaryl are substituted with R^{x}, R^{y}, and R^{y1}.

123. In embodiment 123, the compound is as provided in any one of embodiments 1 to 122, or a pharmaceutically acceptable salt thereof, wherein R⁷ is -Q-(alk¹)ⁿ¹-SO₂R¹¹, -Q²-(alk³)ₙ₃-SO₂NR¹⁴R¹⁵, heterocyclyl, bicyclic heterocyclyl, spiro heterocyclyl, or spiro sulfoximinyl, wherein heterocyclyl, bicyclic heterocyclyl, spiro heterocyclyl, and spiro sulfoximinyl are substituted with R^{x}, R^{y}, and R^{y1}.

124. In embodiment 124, the compound is as provided in any one of embodiments 1 to 122, or a pharmaceutically acceptable salt thereof, wherein R⁷ is-Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, or -Q⁶-(alk⁶)ₙ₇-NR²¹SO₂R²².

125. In embodiment 125, the compound is as provided in any one of embodiments 1 to 122, or a pharmaceutically acceptable salt thereof, wherein R⁷ is heterocyclyl, bicyclic heterocyclyl, fused heterocyclyl, spiro heterocyclyl, bridged heterocyclyl, cyclylsulfoximinyl, bicyclylsulfoximinyl, or spiro sulfoximinyl, wherein heterocyclyl, bicyclic heterocyclyl, fused heterocyclyl, spiro heterocyclyl, bridged heterocyclyl, cyclylsulfoximinyl, bicyclylsulfoximinyl, or spiro sulfoximinyl are substituted with R^{x}, R^{y}, and R^{y1}.

126. In embodiment 126, the compound is as provided in any one of embodiments 1 to 121, or a pharmaceutically acceptable salt thereof, wherein R⁷ is aryl, heteroaryl, or fused heteroaryl, wherein each of the aforementioned ring is substituted with R^{x}, R^{y}, and R^{y1}.

127. In embodiment 127, the compound is as provided in any one of embodiments 1 to 124, or a pharmaceutically acceptable salt thereof, wherein R⁷ is -Q-(alk¹)ₙ₁-SO₂R¹¹.

128. In embodiment 128, the compound is as provided in any one of embodiments 1 to 124, or a pharmaceutically acceptable salt thereof, wherein R⁷ is -Q²-(alk³)ₙ₃-SO₂NR¹⁴R¹⁵.

129. In embodiment 129, the compound is as provided in any one of embodiments 1 to 123 and 125, or a pharmaceutically acceptable salt thereof, wherein R⁷ is heterocyclyl substituted with R^{x}, R^{y}, and R^{y1}.

130. In embodiment 130, the compound is as provided in any one of embodiments 1 to 123 and 125, or a pharmaceutically acceptable salt thereof, wherein R⁷ is bicyclic heterocyclyl substituted with R^{x}, R^{y}, and R^{y1}.

131. In embodiment 131, the compound is as provided in any one of embodiments 1 to 123 and 125, or a pharmaceutically acceptable salt thereof, wherein R⁷ is spiro heterocyclyl substituted with R^{x}, R^{y}, and R^{y1}.

132. In embodiment 132, the compound is as provided in any one of embodiments 1 to 123 and 125, or a pharmaceutically acceptable salt thereof, wherein R⁷ is spiro sulfoximinyl substituted with R^{x}, R^{y}, and R^{y1}.

133. In embodiment 133, the compound is as provided in any one of embodiments 1 to 122 and 124, or a pharmaceutically acceptable salt thereof, wherein R⁷ is -Q¹-(alk³)ₙ₂-SO(═NR¹²)R¹³.

134. In embodiment 134, the compound is as provided in any one of embodiments 1 to 122 and 124, or a pharmaceutically acceptable salt thereof, wherein R⁷ is -Q³-(alk⁴)ₙ₄-COR¹⁶.

135. In embodiment 135, the compound is as provided in any one of embodiments 1 to 122 and 124, or a pharmaceutically acceptable salt thereof, wherein R⁷ is -Q⁴-(alk⁵)ₙ₅-CONR¹⁷R¹⁸.

136. In embodiment 136, the compound is as provided in any one of embodiments 1 to 122 and 124, or a pharmaceutically acceptable salt thereof, wherein R⁷ is -Q⁵-(alk⁶)ₙ₆-NR¹⁹COR²⁰.

137. In embodiment 137, the compound is as provided in any one of embodiments 1 to 122 and 124, or a pharmaceutically acceptable salt thereof, wherein R⁷ is -Q⁶-(alk⁷)ₙ₇-NR²¹SO₂R²².

138. In embodiment 138, the compound is as provided in any one of embodiments 1 to 122 and 126, or a pharmaceutically acceptable salt thereof, wherein R⁷ is phenyl substituted with R^{x}, R^{y}, and R^{y1}.

139. In embodiment 139, the compound is as provided in any one of embodiments 1 to 122 and 126, or a pharmaceutically acceptable salt thereof, wherein R⁷ is 6-membered heteroaryl substituted with R^{x}, R^{y}, and R^{y1}.

140. In embodiment 140, the compound is as provided in any one of embodiments 1 to 124, 127, 128, and 133 to 137, or a pharmaceutically acceptable salt thereof, is wherein each of Q Q¹, Q², Q³, Q⁴, Q⁵, and Q⁶ is cycloalkyl, bridged cycloalkyl, spiro cycloalkyl, heterocyclyl^{A}, bicyclic heterocyclyl^{A}, bridged heterocyclyl^{A}, spiro heterocyclyl^{A}, aryl, or heteroaryl, where each of the aforementioned rings is substituted with R^{v} and R^{w}.

141. In embodiment 141, the compound is as provided in any one of embodiments 1 to 124, 127, 128, 133 to 137, and 140, or a pharmaceutically acceptable salt thereof, wherein each of Q, Q¹, Q², Q³, Q⁴, Q⁵, and Q⁶ is cycloalkyl, bridged cycloalkyl, or spiro cycloalkyl, where each of the aforementioned rings is substituted with R^{v} and R^{w}.

142. In embodiment 142, the compound is as provided in any one of embodiments 1 to 124, 127, 128, 133 to 137, 140, and 141, or a pharmaceutically acceptable salt thereof, wherein each of Q, Q¹, Q², Q³, Q⁴, Q⁵, and Q⁶ is cycloalkyl substituted with R^{v} and R^{w}.

143. In embodiment 143, the compound is as provided in any one of embodiments 1 to 124, 127, 128, 133 to 137, 140, and 141, or a pharmaceutically acceptable salt thereof, wherein each of Q, Q¹, Q², Q³, Q⁴, Q⁵, and Q⁶ is bridged cycloalkyl substituted with R^{v} and R^{w}.

144. In embodiment 144, the compound is as provided in any one of embodiments 1 to 124, 127, 128, 133 to 137, 140, and 141, or a pharmaceutically acceptable salt thereof, wherein each of Q, Q¹, Q², Q³, Q⁴, Q⁵, and Q⁶ is spiro cycloalkyl substituted with R^{v} and R^{w}.

145. In embodiment 145, the compound is as provided in any one of embodiments 1 to 124, 127, 128, 133 to 137, and 140, or a pharmaceutically acceptable salt thereof, wherein each of Q, Q¹, Q², Q³, Q⁴, Q⁵, and Q⁶ is heterocyclyl^{A}, bicyclic heterocyclyl^{A}, bridged heterocyclyl^{A}, or spiro heterocyclyl^{A}, where each of the aforementioned rings is substituted with R^{v} and R^{w}.

146. In embodiment 146, the compound is as provided in any one of embodiments 1 to 124, 127, 128, 133 to 137, 140, and 145, or a pharmaceutically acceptable salt thereof, wherein each of Q, Q¹, Q², Q³, Q⁴, Q⁵, and Q⁶ is heterocyclyl^{A} or bicyclic heterocyclyl^{A}, where each of the aforementioned rings is substituted with R^{v} and R^{w}.

147. In embodiment 147, the compound is as provided in any one of embodiments 1 to 124, 127, 128, 133 to 137, 140, 145, and 146, or a pharmaceutically acceptable salt thereof, wherein each of Q, Q¹, Q², Q³, Q⁴, Q⁵, and Q⁶ is heterocyclyl^{A} substituted with R^{v} and R^{w}.

148. In embodiment 148, the compound is as provided in any one of embodiments 1 to 124, 127, 128, 133 to 137, 140, 145, and 146, or a pharmaceutically acceptable salt thereof, wherein each of Q, Q¹, Q², Q³, Q⁴, Q⁵, and Q⁶ is bicyclic heterocyclyl^{A} substituted with R^{v} and R^{w}.

149. In embodiment 149, the compound is as provided in any one of embodiments 1 to 124, 127, 128, 133 to 137, 140, and 145, or a pharmaceutically acceptable salt thereof, wherein each of Q, Q¹, Q², Q³, Q⁴, Q⁵, and Q⁶ is bridged heterocyclyl^{A} or spiro heterocyclyl^{A}, where each of the aforementioned rings is substituted with R^{v} and R^{w}.

150. In embodiment 150, the compound is as provided in any one of embodiments 1 to 124, 127, 128, 133 to 137, 140, 145, and 149, or a pharmaceutically acceptable salt thereof, wherein each of Q, Q¹, Q², Q³, Q⁴, Q⁵, and Q⁶ is spiro heterocyclyl^{A} substituted with R^{v} and R^{w}.

151. In embodiment 151, the compound is as provided in any one of embodiments 1 to 124, 127, 128, 133 to 137, and 140, or a pharmaceutically acceptable salt thereof, wherein each of Q, Q¹, Q², Q³, Q⁴, Q⁵, and Q⁶ is aryl or heteroaryl, where each of the aforementioned rings is substituted with R^{v} and R^{w}.

152. In embodiment 152, the compound is as provided in any one of embodiments 1 to 124, 127, 128, 133 to 137, 140, and 151, or a pharmaceutically acceptable salt thereof, wherein each of Q, Q¹, Q², Q³, Q⁴, Q⁵, and Q⁶ is phenyl or 6-membered heteroaryl, where each of the aforementioned rings is substituted with R^{y} and R^{w}.

153. In embodiment 153, the compound is as provided in any one of embodiments 1 to 124, 127, 128, 133 to 137, 140, 151, and 152, or a pharmaceutically acceptable salt thereof, wherein each of Q, Q¹, Q², Q³, Q⁴, Q⁵, and Q⁶ is phenyl substituted with R^{v} and R^{w}.

154. In embodiment 154, the compound is as provided in any one of embodiments 1 to 124, 127, 128, 133 to 137, and 140 to 153, or a pharmaceutically acceptable salt thereof, wherein each of n¹, n², n³, n⁴, n⁵, n⁶, and n⁷ is 0.

155. In embodiment 155, the compound is as provided in any one of embodiments 1 to 124, 127, 128, 133 to 137, and 140 to 153, or a pharmaceutically acceptable salt thereof, wherein each of n¹, n², n³, n⁴, n⁵, n⁶, and n⁷ is 1.

156. In embodiment 156, the compound is as provided in any one of embodiments 1 to 124, 127, 128, 133 to 137, 140 to 153, and 155, or a pharmaceutically acceptable salt thereof wherein each of alk¹, alk², alk³, alk⁴, alk⁵, alk⁶, and alk⁷ is methylene or ethylene.

157. In embodiment 157, the compound is as provided in any one of embodiments 1 to 124, 127, 128, 133 to 137, 140 to 153, and 155, or a pharmaceutically acceptable salt thereof, wherein each of alk¹, alk², alk³, alk⁴, alk⁵, alk⁶, and alk⁷ is methylene.

158. In embodiment 158, the compound is as provided in any one of embodiments 1 to 124, 127, 128, 133 to 137, 140 to 153, and 155, or a pharmaceutically acceptable salt thereof, wherein each of alk¹, alk², alk³, alk⁴, alk⁵, alk⁶, and alk⁷ is ethylene, propylene, butylene, or pentylene.

159. In embodiment 159, the compound is as provided in any one of embodiments 1 to 124, 127, 128, 133 to 137, 140 to 153, 155, and 158, or a pharmaceutically acceptable salt thereof, wherein each of alk¹, alk², alk³, alk⁴, alk⁵, alk⁶, and alk⁷ is ethylene or butylene.

160. In embodiment 160, the compound is as provided in any one of embodiments 1 to 159, or a pharmaceutically acceptable salt thereof, wherein R⁷ is where:
(1). each of Q, Q¹, Q², Q³, Q⁴, Q⁵, and Q⁶ is:

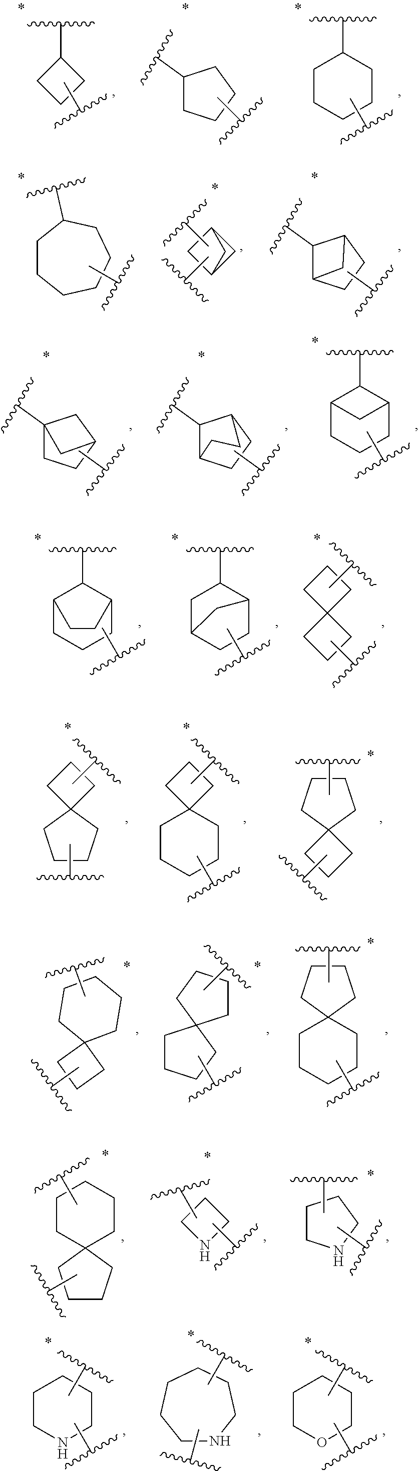

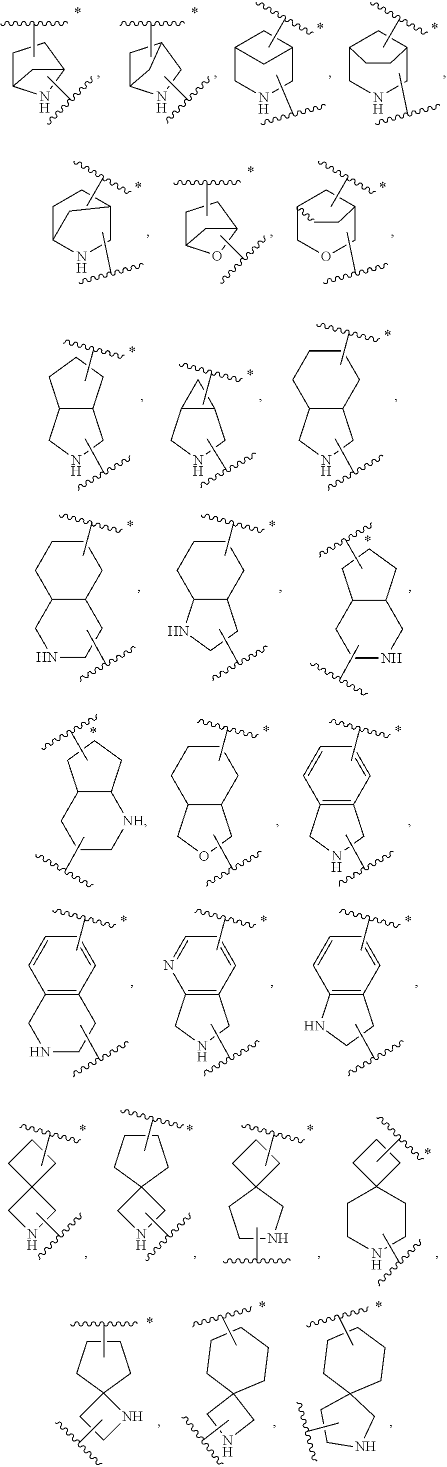

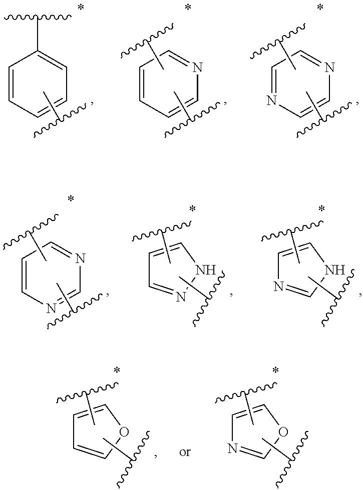

wherein each ring of Q, Q¹, Q², Q³, Q⁴, Q⁵, and Q⁶ is substituted as defined therein; and
(2). the heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
bridged heterocyclyl is:

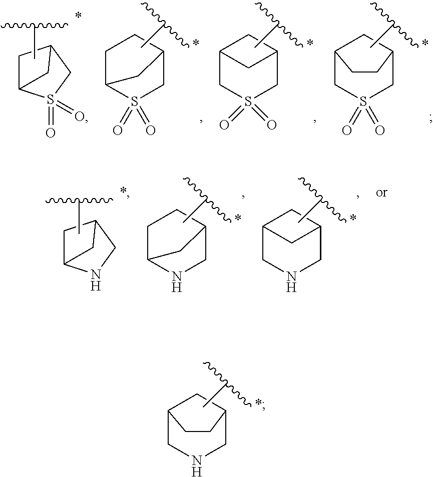

bicyclic heterocyclyl is:

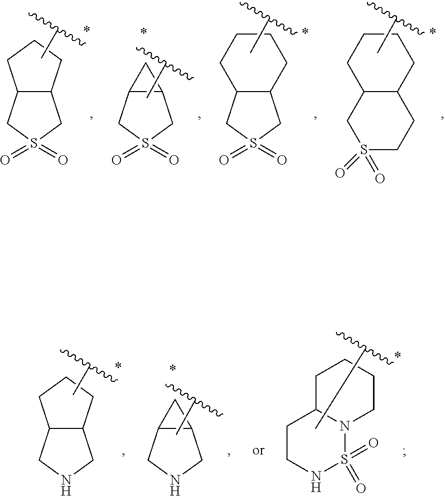

spiro heterocyclyl is:

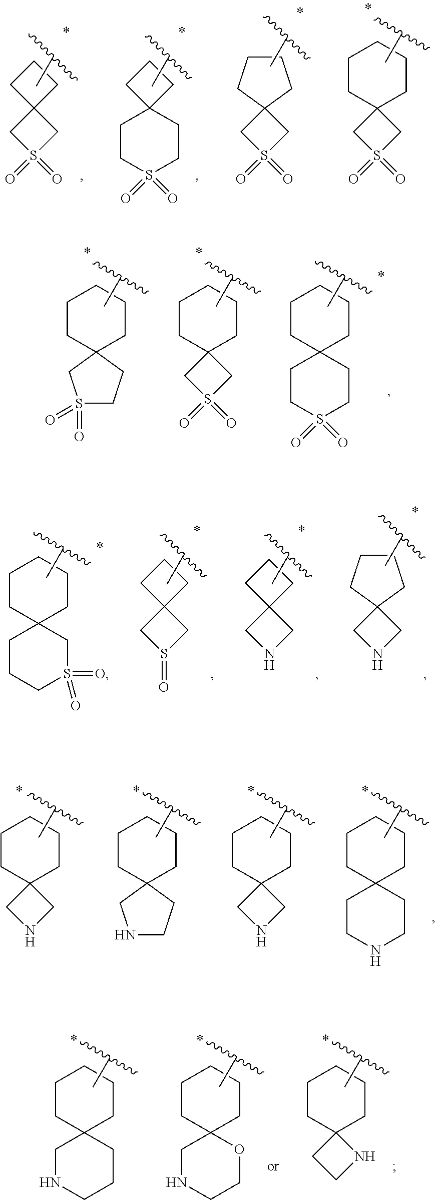

heterocyclyl is:

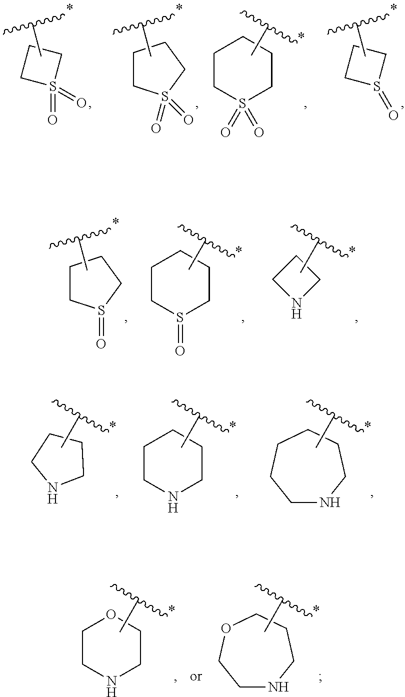

spiro sulfoximinyl is:

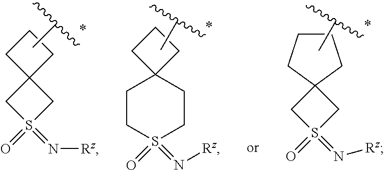

cyclylsulfoximinyl is:

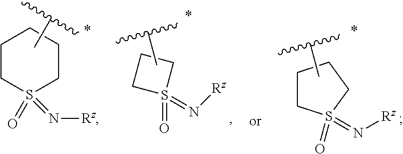

aryl is:

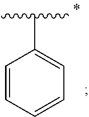 [SMILES: *c1ccccc1]

heteroaryl is:

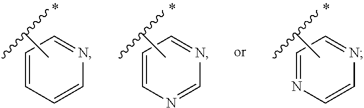

and
fused heteroaryl is:

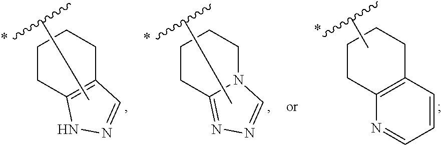

wherein each ring in (2) is substituted as defined therein; and
wherein in each ring in (1) and (2) above,

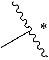

denotes bond to NH of —NHR⁷ moiety in the compound of Formula (I); and

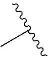

in each ring in (1) denotes bond to remainder of -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, or -Q⁶-(alk⁶)ₙ₇-NR²¹SO₂R²².

161. In embodiment 161, the compound is as provided in any one of embodiments 1 to 160, or a pharmaceutically acceptable salt thereof, is wherein R⁷ is where:
(1). each of Q, Q¹, Q², Q³, Q⁴, Q⁵, and Q⁶ is:

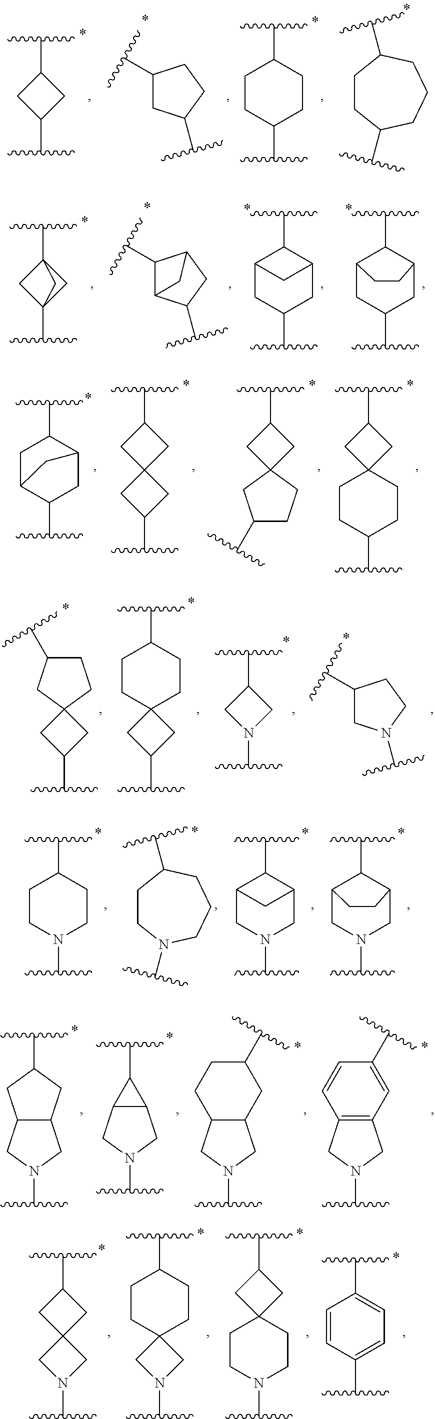

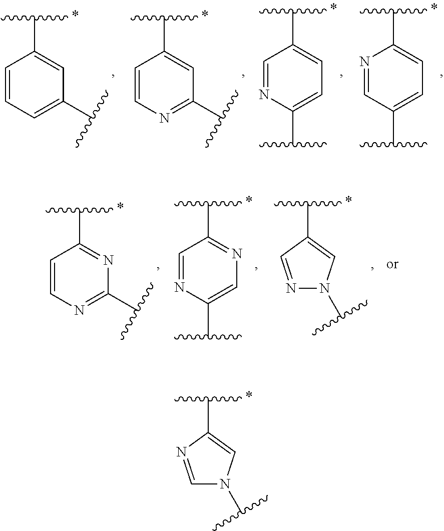

wherein each ring of Q, Q¹, Q², Q³, Q⁴, Q⁵, and Q⁶ is substituted as defined therein; and
(2). the heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is:

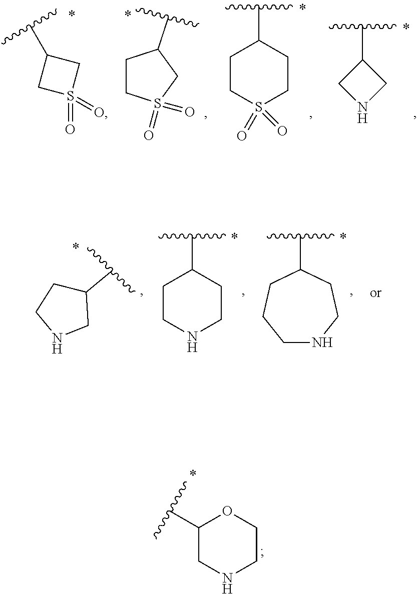

spiro heterocyclyl is:

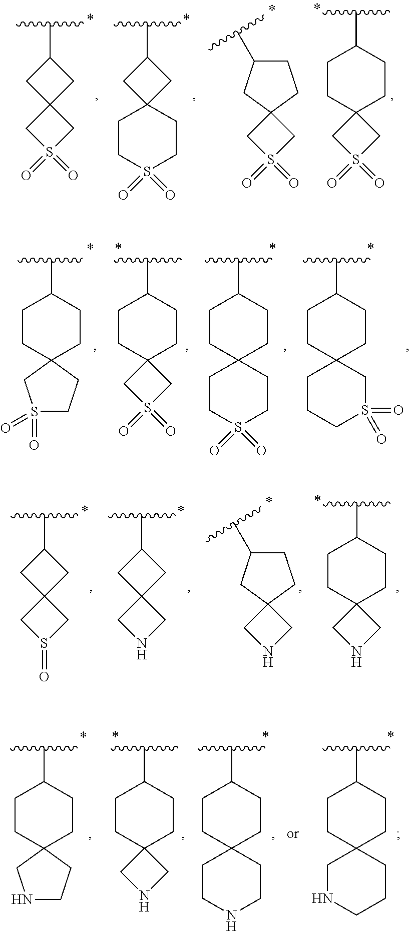

bridged heterocyclyl is:

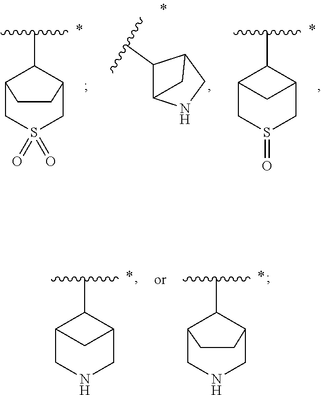

bicyclic heterocyclyl is:

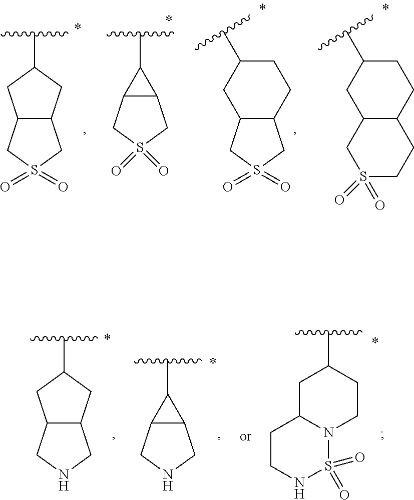

spiro sulfoximinyl is:

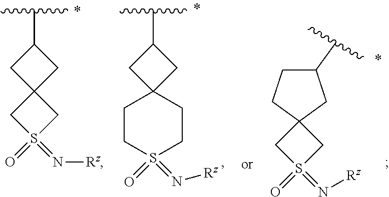

cyclylsulfoximinyl is:

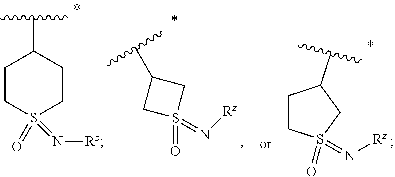

aryl is:

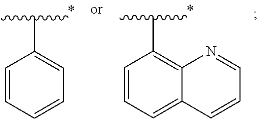 [SMILES: *c1ccccc1 ; *c1cccc2cccnc12]

heteroaryl is:

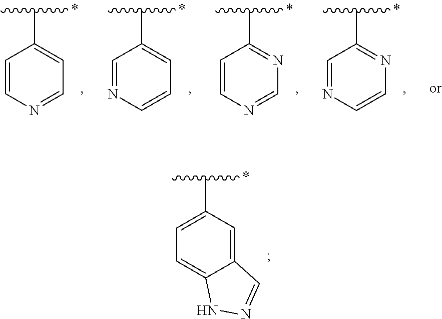 [SMILES: *c1ccncc1 ; *c1cccnc1 ; *c1ccncn1 ; *c1cnccn1 ; *c1ccc2[nH]ncc2c1]

fused heteroaryl is:

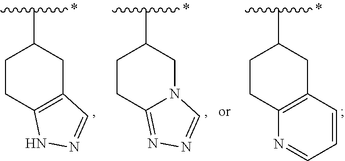

wherein each ring in (2) is substituted as defined therein; and
wherein

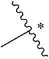

in each ring in (1) and (2) and

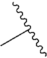

in each ring in (1) have the same meaning as in embodiment 160.

162. In embodiment 162, the compound is as provided in any one of embodiments 1 to 159, and 161, or a pharmaceutically acceptable salt thereof, wherein R⁷ is where:
(1). each of Q, Q¹, Q², Q³, Q⁴, Q⁵, and Q⁶ is:

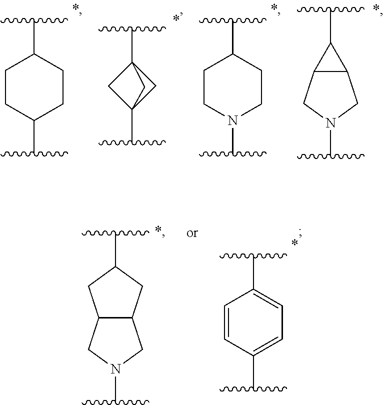

wherein each ring of Q, Q¹, Q², Q³, Q¹, Q′, and QG is substituted as defined therein; and
(2). heterocyclyl, bicyclic heterocyclyl, spiro heterocyclyl, spiro sulfoximinyl, aryl, and heteroaryl of R⁷ are where:
heterocyclyl is:

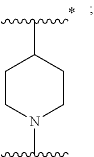 [SMILES: *C1CCN(*)CC1]

bicyclic heterocyclic is:

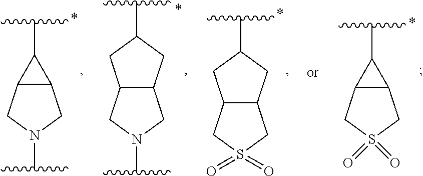 [SMILES: *C1C2CN(*)CC12 ; *C1CC2CN(*)CC2C1 ; *C1CC2CS(=O)(=O)CC2C1 ; *C1C2CS(=O)(=O)CC12]

spiro heterocyclyl is:

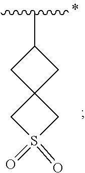 [SMILES: *C1CC2(C1)CS(=O)(=O)C2]

spirosulfoximinyl is:

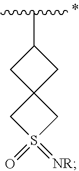

aryl is:

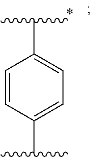 [SMILES: *c1ccc(*)cc1]

and heteroaryl is:

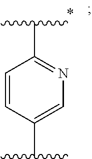 [SMILES: *c1ccc(*)nc1]

wherein each ring in (2) is substituted as defined therein; and
wherein

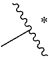

in each ring in (1) and (2); and

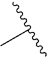

in each ring in (1) have the same meaning as in embodiment 160 and

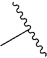

in each ring in (2) denotes attachment to one of R^{x}, R^{y}, and R^{y1}.

163. In embodiment 163, the compound is as provided in any one of embodiments 1 to 124, 127, 128, 133 to 137 and 140 to 159, or a pharmaceutically acceptable salt thereof, wherein each of Q, Q¹, Q², Q³, Q⁴, Q⁵, and Q⁶ is:

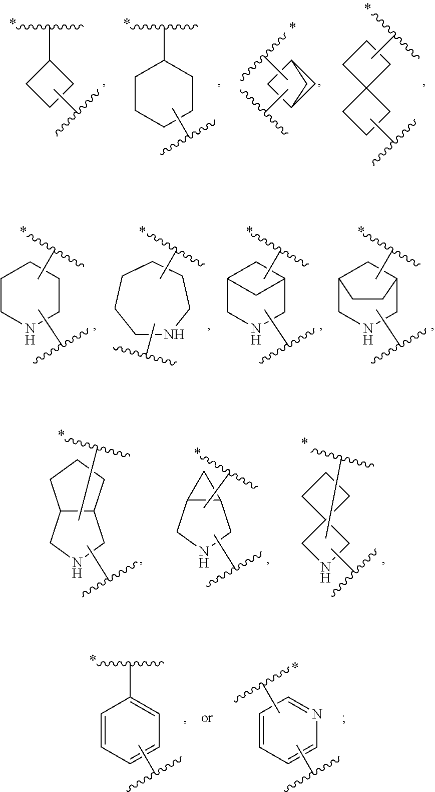

wherein each ring is substituted as defined therein and wherein

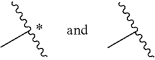

in each ring have the same meaning as in embodiment 160.

164. In embodiment 164, the compound is as provided in any one of embodiments 1 to 124, 127, 128, 133 to 137, 140 to 160 or 163, or a pharmaceutically acceptable salt thereof, wherein each of Q, Q¹, Q², Q³, Q⁴, Q⁵, and Q⁶ is:

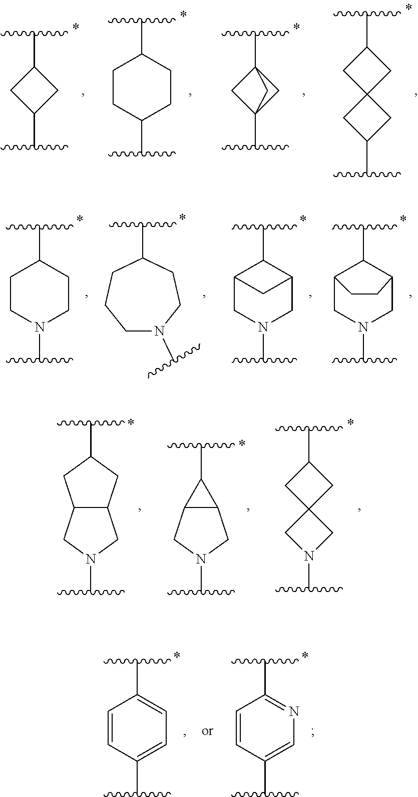

where each ring is substituted as defined therein; and
wherein

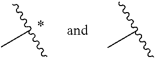

in each ring have the same meaning as in embodiment 160.

165. In embodiment 165, the compound is as provided in any one of embodiments 1 to 124, 127, 128, 133 to 137, 140 to 143, 145 to 148, 151 to 159, and 164, or a pharmaceutically acceptable salt thereof, wherein each of Q, Q¹, Q², Q³, Q⁴, Q⁵, and Q⁶ is:

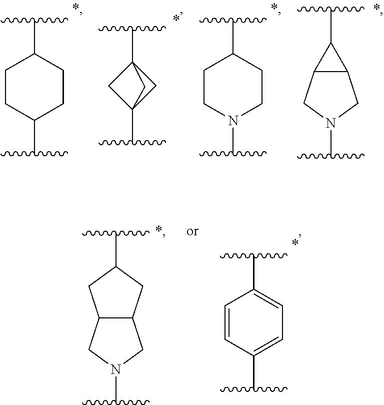

where each ring is substituted as defined therein; and
wherein

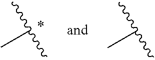

in each ring have the same meaning as in embodiment 160.

166. In embodiment 166, the compound is as provided in any one of embodiments 1 to 124, 127, 128, 133 to 137, 140 to 143, 154 to 159, 164 and 165, or a pharmaceutically acceptable salt thereof, wherein each of Q, Q¹, Q², Q³, Q⁴, Q⁵, and Q⁶ is:

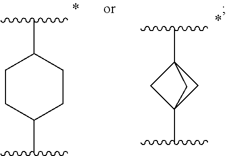 [SMILES: *C1CCC(*)CC1 ; *C12CC(*)(C1)C2]

and each ring is substituted as defined therein; and
wherein

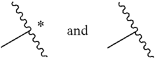

in each ring have the same meaning as in embodiment 160.

167. In embodiment 167, the compound is as provided in any one of embodiments 1 to 124, 127, 128, 133 to 137, 140, 145 to 147, 154 to 159, 164, and 165, or a pharmaceutically acceptable salt thereof, wherein each of Q, Q¹, Q², Q³, Q⁴, Q⁵, and Q⁶ is:

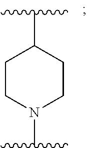 [SMILES: *C1CCN(*)CC1]

and is substituted as defined therein; and
wherein

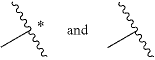

in each ring have the same meaning as in embodiment 160.

168. In embodiment 168, the compound is as provided in any one of embodiments 1 to 124, 127, 128, 133 to 137, 140, 145, 146, 148, 154 to 159, 164, and 165, or a pharmaceutically acceptable salt thereof, wherein each of Q, Q¹, Q², Q³, Q⁴, Q⁵, and Q⁶ is:

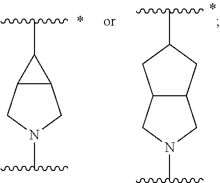 [SMILES: *C1C2CN(*)CC12 ; *C1CC2CN(*)CC2C1]

and each ring is substituted as defined therein; and
wherein

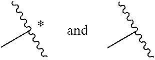

in each ring have the same meaning as in embodiment 160.

169. In embodiment 169, the compound is as provided in any one of embodiments 1 to 124, 127, 128, 133 to 137, 140, 151 to 159,164, and 165, or a pharmaceutically acceptable salt thereof, wherein each of Q, Q¹, Q², Q³, Q⁴, Q⁵, and Q⁶ is:

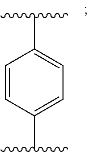 [SMILES: *c1ccc(*)cc1]

and is substituted as defined therein; and
wherein

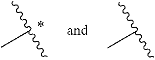

have the same meaning as in embodiment 160.

169a. In embodiment 169a, the compound is as provided in any one of embodiments 1 to 124, 127, 128, 133 to 137, 140, 143, 154 to 159, and 164 to 166, or a pharmaceutically acceptable salt thereof, wherein each of Q, Q¹, Q², Q³, Q⁴, Q⁵, and Q⁶ is:

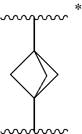 [SMILES: *C12CC(*)(C1)C2]

and is substituted as defined therein; and
wherein

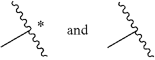

have the same meaning as in embodiment 160.

170. In embodiment 170, the compound is as provided in any one of embodiments 1 to 123, 125, 126, 129 to 132, 138, 139, and 154 to 169a, or a pharmaceutically acceptable salt thereof, wherein the heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is:

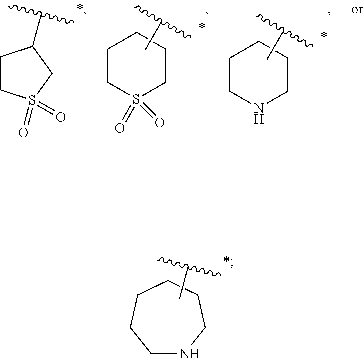

spiro heterocyclyl is:

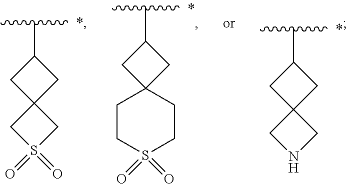 [SMILES: *C1CC2(C1)CS(=O)(=O)C2 ; *C1CC2(CCS(=O)(=O)CC2)C1 ; *C1CC2(CNC2)C1]

bridged heterocyclyl is:

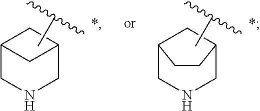

bicyclic heterocyclyl is:

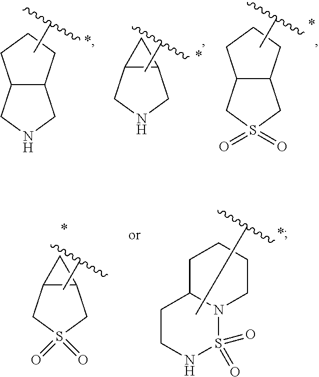

cyclylsulfoximinyl is:

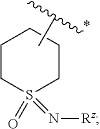

spiro sulfoximinyl is:

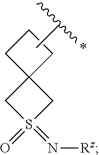

aryl is:

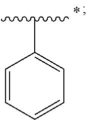 [SMILES: *c1ccccc1]

heteroaryl is:

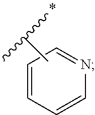

and fused heteroaryl is:

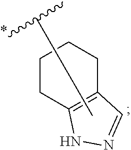

wherein each ring is substituted as defined therein; and
wherein

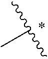

in each ring has the same meaning as in embodiment 160.

171. In embodiment 171, the compound is as provided in any one of embodiments 1 to 123, 125, 126, 129 to 132, 138, 139, and 154 to 170, or a pharmaceutically acceptable salt thereof; wherein the heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is:

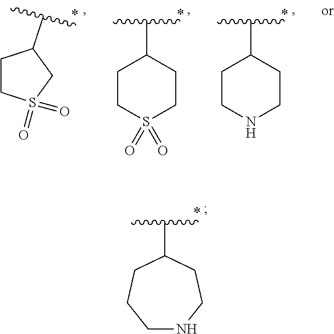

spiro heterocyclyl is:

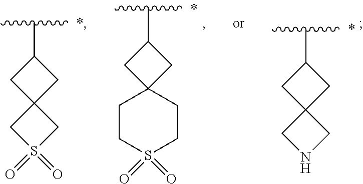 [SMILES: *C1CC2(C1)CS(=O)(=O)C2 ; *C1CC2(CCS(=O)(=O)CC2)C1 ; *C1CC2(CNC2)C1]

bridged heterocyclyl is:

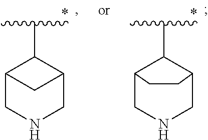 [SMILES: *C1C2CNCC1C2 ; *C1C2CCC1CNC2]

bicyclic heterocyclyl is:

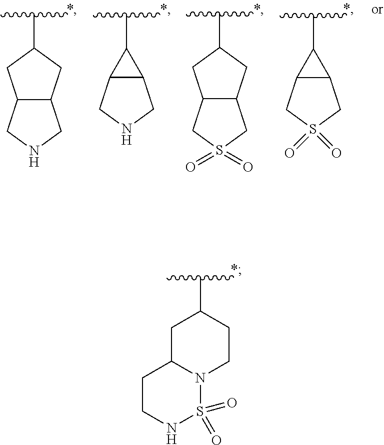

cyclylsulfoximinyl is:

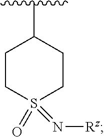

spiro sulfoximinyl is:

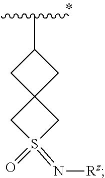

aryl is:

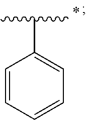 [SMILES: *c1ccccc1]

heteroaryl is:

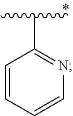 [SMILES: *c1ccccn1]

and fused heteroaryl is:

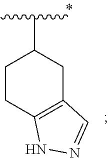 [SMILES: *C1CCc2[nH]ncc2C1]

and each ring is substituted as defined therein; and
wherein

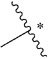

in each ring have the same meaning as in embodiment 160.

172. In embodiment 172, the compound is as provided in any one of embodiments 1 to 123, 125, 126, 129 to 132, 138, 139, 154 to 169a, and 171, or a pharmaceutically acceptable salt thereof, wherein the heterocyclyl, bicyclic heterocyclyl, spiro heterocyclyl, spiro sulfoximinyl, aryl, and heteroaryl, of R⁷ are where:
heterocyclyl is:

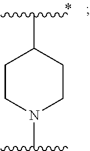 [SMILES: *C1CCN(*)CC1]

bicyclic heterocyclic is:

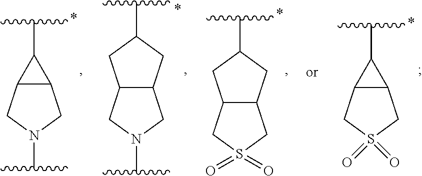 [SMILES: *C1C2CN(*)CC12 ; *C1CC2CN(*)CC2C1 ; *C1CC2CS(=O)(=O)CC2C1 ; *C1C2CS(=O)(=O)CC12]

spiro heterocyclyl is:

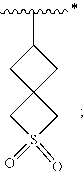 [SMILES: *C1CC2(C1)CS(=O)(=O)C2]

spirosulfoximinyl is:

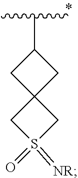

aryl is:

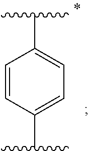 [SMILES: *c1ccc(*)cc1]

and heteroaryl is:

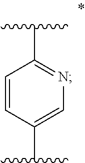 [SMILES: *c1ccc(*)nc1]

where each ring is substituted as defined therein; and
wherein

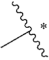

in each ring have the same meaning as in embodiment 160.

173. In embodiment 173, the compound is as provided in any one of embodiments 1 to 123, 125, 126, 129 to 132, 138, 139, 154 to 169a, 171, and 172, or a pharmaceutically acceptable salt thereof, wherein heterocyclyl of R⁷ is: heterocyclyl is:

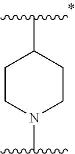 [SMILES: *C1CCN(*)CC1]

and is substituted as defined therein; and
wherein

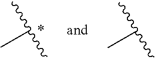

have the same meaning as in embodiment 162.

174. In embodiment 174, the compound is as provided in any one of embodiments 1 to 123, 125, 126, 129 to 132, 138, 139, 154 to 169a, 171, and 172, or a pharmaceutically acceptable salt thereof, wherein spiro heterocyclyl, of R⁷ is

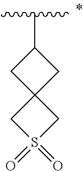 [SMILES: *C1CC2(C1)CS(=O)(=O)C2]

and is substituted as defined therein; and wherein

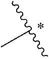

has the same meaning as in embodiment 160.

175. In embodiment 175, the compound is as provided in any one of embodiments 1 to 123, 125, 126, 129 to 132, 138, 139, 154 to 169a, 171, and 172, or a pharmaceutically acceptable salt thereof, is wherein bicyclic heterocyclyl, of R⁷ is: bicyclic heterocyclic is:

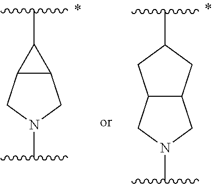 [SMILES: *C1C2CN(*)CC12 ; *C1CC2CN(*)CC2C1]

and each ring is substituted as defined therein; and
wherein

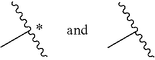

in each ring have the same meaning as in embodiment 162.

175a. In embodiment 175a, the compound is as provided in any one of embodiments 1 to 123, 125, 126, 129 to 132, 138, 139, 154 to 169a, 171, and 172, or a pharmaceutically acceptable salt thereof, wherein aryl of R⁷ is:

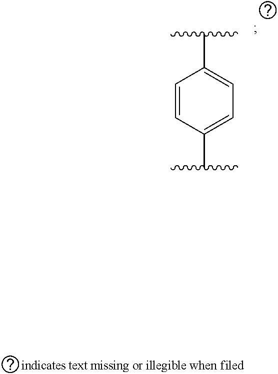 [SMILES: *c1ccc(*)cc1]

and is substituted as defined therein; and
wherein

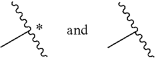

have the same meaning as in embodiment 162.

176 In embodiment 176, the compound is as provided in any one of embodiments 1 to 123, 125, 126, 129 to 132, 138, 139, 154 to 169a, 171, and 172 or a pharmaceutically acceptable salt thereof, wherein bicyclic heterocyclyl of R⁷ is

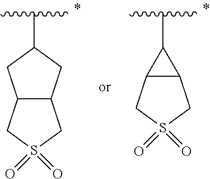 [SMILES: *C1CC2CS(=O)(=O)CC2C1 ; *C1C2CS(=O)(=O)CC12]

and each ring is substituted with R^{x}, R^{y}, and R^{y1}; and
wherein

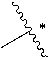

in each ring has the same meaning as in embodiment 160.

177. In embodiment 177, the compound is as provided in any one of embodiments 1 to 124, 127, 128, and 133 to 137, and 170 to 176, or a pharmaceutically acceptable salt thereof, wherein each of Q, Q¹, Q², Q³, Q⁴, Q⁵, and Q⁶ is a bond.

178. In embodiment 178, the compound is as provided in any one of embodiments 1 to 124, 127, 128, 133 to 137, and 170 to 177, or a pharmaceutically acceptable salt thereof, wherein each of alk², alk³, alk⁴, alk⁵, alk⁶, and alk⁷ is ethylene, propylene, butylene, or pentylene.

179. In embodiment 179, the compound is as provided in any one of embodiments 1 to 124, 127, 128, 133 to 137, and 170 to 178, or a pharmaceutically acceptable salt thereof, wherein each of alk², alk³, alk⁴, alk⁵, alk⁶, and alk⁷ is ethylene or butylene.

180. In embodiment 180, the compound is as provided in any one of embodiments 1 to 122, 124, 133, 136, 137, and 140 to 179, or a pharmaceutically acceptable salt thereof, wherein R¹², R¹⁹, and R²¹ are hydrogen, methyl, ethyl, or propyl.

181. In embodiment 181, the compound is as provided in any one of embodiments 1 to 122, 124, 133, 136, 137, and 140 to 180, or a pharmaceutically acceptable salt thereof, wherein R¹², R¹⁹, and R²¹ are hydrogen.

182. In embodiment 182, the compound is as provided in any one of embodiments 1 to 122, 124, 133, 136, 137, and 140 to 180, or a pharmaceutically acceptable salt thereof, wherein R¹², R¹⁹, and R²¹ are methyl, ethyl, or propyl.

183. In embodiment 183, the compound is as provided in any one of embodiments 1 to 124, 127, 128, 133 to 137, and 140 to 182, or a pharmaceutically acceptable salt thereof, wherein
R¹¹, R¹⁶, R²⁰, and R²² are independently selected from alkyl, fluoro, chloro, —CR²³═CR²⁴R²⁵, or heterocyclyl substituted with one to three substituents independently selected from hydrogen, alkyl, alkoxy, hydroxy, halo, haloalkyl, haloalkoxy, hydroxyalkyl, alkoxyalkyl, aminoalkyl, cyano, and heterocyclyl;
R¹³ is hydrogen, alkyl, haloalkyl, hydroxyalkyl, alkoxyalkyl, aminoalkyl, or cycloalkyl; and
R¹⁴, R¹⁵, R¹⁷, and R¹⁸ are independently selected from hydrogen, alkyl, hydroxyalkyl, alkoxyalkyl, aminoalkyl, or heterocyclyl which is substituted with one to three substituents independently selected from hydrogen, alkyl, alkoxy, hydroxy, halo, haloalkyl, haloalkoxy, and cyano.

184. In embodiment 184, the compound is as provided in any one of embodiments 1 to 124, 127, 128, and 140 to 183, or a pharmaceutically acceptable salt thereof, wherein R¹¹ is methyl, ethyl, piperazinyl, 4-methylpiperazinyl, or 4-ethylpiperzin-1-yl; R¹⁴ is hydrogen, methyl, or ethyl, R¹⁵ is hydrogen, methyl, ethyl, aminoethyl, methylaminoethyl, dimethylaminoethyl, diethylaminoethyl, azetidine-3-yl, 1-methylazetidin-3-yl, 1-ethylazetidin-2-yl, 1-methylpyrrolidin-3-yl, 1-ethylpyrrolidin-3-yl, 1-methylpiperidinyl, or 1-ethylpiperidinyl.

185. In embodiment 185, the compound is as provided in any one of embodiments 1 to 124, 127, 128, and 140 to 184, or a pharmaceutically acceptable salt thereof, wherein R¹¹ is methyl.

186. In embodiment 186, the compound is as provided in any one of embodiments 1 to 123, 125, 126, 129 to 132, and 138 to 186, or a pharmaceutically acceptable salt thereof, wherein R^{x}, R^{y}, and R^{y1} where R^{x} and R^{y} are independently selected from hydrogen, deuterium, alkyl, alkoxy, hydroxy, halo, haloalkyl, haloalkoxy, amino, alkylamino, dialkylamino, and cyano and R^{y1} is hydrogen, deuterium, alkyl, alkoxy, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, heterocyclylalkyloxy (wherein aryl, by itself or as part of aralkyl or aryloxy, heteroaryl, by itself or as part, of heteroaryloxy and heteroaralkyl, and heterocyclyl, by itself or as part of, heterocyclylalkyl, heterocyclyloxy, or heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl).

187. In embodiment 187, the compound is as provided in any one of embodiments 1 to 123, 125, 126, 129 to 133, and 138 to 186, or a pharmaceutically acceptable salt thereof, wherein R^{x} and R^{y} are independently selected from hydrogen, deuterium, alkyl, halo, haloalkyl, haloalkoxy, and cyano and R^{y1} is hydrogen, deuterium, alkyl, alkoxy, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, heterocyclylalkyloxy, (wherein aryl, by itself or as part of aralkyl or aryloxy, heteroaryl, by itself or as part, of heteroaryloxy and heteroaralkyl, and heterocyclyl, by itself or as part of, heterocyclylalkyl, heterocyclyloxy, or heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl).

188. In embodiment 188, the compound is as provided in any one of embodiments 1 to 123, 125, 126, 129 to 132, and 138 to 187, or a pharmaceutically acceptable salt thereof, wherein R^{x} and R^{y} are independently selected from hydrogen, deuterium, and alkyl and R^{y1} is hydrogen, deuterium, alkyl, alkoxy, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, or heterocyclylalkyloxy, (wherein aryl, by itself or as part of aralkyl or aryloxy, heteroaryl, by itself or as part, of heteroaryloxy and heteroaralkyl, and heterocyclyl, by itself or as part of, heterocyclylalkyl, heterocyclyloxy, or heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl).

189. In embodiment 189, the compound is as provided in any one of embodiments 1 to 123, 125, 126, 129 to 132, and 138 to 188, or a pharmaceutically acceptable salt thereof, wherein R^{x} and R^{y} are independently selected from hydrogen or methyl and R^{y1} is hydrogen, deuterium, acyl, aminocarbonyl, aminocarbonylalkyl, aminoalkyl, or heterocyclyl, wherein heterocyclyl is substituted with one to three substituents independently selected from hydrogen, alkyl, alkoxycarbonyloxy, alkoxyalkyl, alkoxyalkyloxyalkyl, and heterocyclyl.

190. In embodiment 190, the compound is as provided in any one of embodiments 1 to 121, or a pharmaceutically acceptable salt thereof, wherein R⁷ is 2,2-dioxido-2-thiaspiro[3.3]heptan-6-yl, 3,3-dioxido-3-thiabicyclo[3.1.0]hexan-6-yl, 4-(methylsulfonyl)-cyclohexyl, 2,2-dioxidohexahydro-1H-cyclopenta[c]thiophen-5-yl, 1-(methylsulfonyl)piperidin-4-yl, 1-(methylaminosulfonyl)piperidin-4-yl, 1-(aminosulfonyl)piperidin-4-yl, 4-(piperazin-1-yl)phenyl, 4-(piperazin-1-ylsulfonyl)piperidin-4-yl, 1-(N-(2-dimethylaminoethyl)-N-methylaminosulfonyl)piperidin-4-yl, 4-(4-methylpiperazin-1-ylsulfonyl)piperidin-4-yl, 5-(piperazin-1-yl)pyridin-2-yl, 4-(piperazin-1-yl)phenyl, 1-(N-(azetidin-3-yl)-N-methylamino-sulfonyl)piperidin-4-yl, 1-(N-(1-methylazetidin-3-yl)-N-methylaminosulfonyl)piperidin-4-yl, 2-(methylsulfonyl)-2-azaspiro[3.3]heptan-6-yl, 4-(4-(2-methoxyethyl)piperazin-1-yl)phenyl, 4-(2-(2-methoxyethoxy)ethyl)piperazin-1-yl)phenyl, 4-(4-(2~ (2-methoxyethoxy)ethyl)piperazin-1-yl)phenyl, 3-dimethylaminomethylphenyl, 4-methylsulfonylphenyl, 4-dimethylaminomethylphenyl, 4-(4-methylpiperazin-1-yl)phenyl, 4-(4-(oxetan-3-yl)piperazin-1-yl)phenyl, 1-(piperazin-1-ylsulfonyl)piperidin-4-yl, 3-((methylsulfonyl)methyl)bicyclo[1.1.1]pentan-1-yl, or 2-imino-2-oxido-N-(3-(2-(bicyclo[1.1.1]pentan-1-yl)-5-(2-((2-imino-2-oxido-λ⁶-thiaspiro[3.3]heptan-6-yl.

191. In embodiment 191, the compound is as provided in any one of embodiments 1 to 121 and 190, or a pharmaceutically acceptable salt thereof, wherein R⁷ is 4-(methylsulfonyl)-cyclohexyl, 1-(methylsulfonyl)piperidin-4-yl, 1-(methylaminosulfonyl)piperidin-4-yl, 1-(aminosulfonyl)piperidin-4-yl, 4-(piperazin-1-ylsulfonyl)piperidin-4-yl, 1-(N-(2-dimethylaminoethyl)-N-methylaminosulfonyl)piperidin-4-yl, 4-(4-methylpiperazin-1-ylsulfonyl)piperidin-4-yl, 1-(N-(azetidin-3-yl)-N-methylaminosulfonyl)piperidin-4-yl, 1-(N-(1-methylazetidin-3-yl)-N-methylaminosulfonyl)piperidin-4-yl, 2-(methylsulfonyl)-2-azaspiro[3.3]heptan-6-yl, 4-methylsulfonylphenyl, 1-(piperazin-1-ylsulfonyl)piperidin-4-yl, or 3-((methylsulfonyl)methyl)bicyclo[1.1.1]pentan-1-yl.

192. In embodiment 192, the compound is as provided in any one of embodiments 1 to 121 and 190, or a pharmaceutically acceptable salt thereof, wherein R⁷ is 2,2-dioxido-2-thiaspiro[3.3]heptan-6-yl, 3,3-dioxido-3-thiabicyclo[3.1.0]hexan-6-yl, 2,2-dioxidohexahydro-1H-cyclopenta[c]thiophen-5-yl, 4-(piperazin-1-yl)phenyl, 5-(piperazin-1-yl)pyridin-2-yl, 4-(piperazin-1-yl)phenyl, 4-(4-(2-methoxyethyl)piperazin-1-yl)phenyl, 4-(2-(2-methoxyethoxy)ethyl)piperazin-1-yl)phenyl, 4-(4-(2-(2-methoxyethoxy)ethyl)piperazin-1-yl)phenyl, 3-dimethylaminomethylphenyl, 4-dimethylaminomethylphenyl, 4-(4-methylpiperazin-1-yl)phenyl, 4-(4-(oxetan-3-yl)piperazin-1-yl)phenyl, or 2-imino-2-oxido-λ⁶-thiaspiro[3.3]heptan-6-yl.

193. In embodiment 193, the compound is as provided in any one of embodiments 1 to 121 and 190, or a pharmaceutically acceptable salt thereof, wherein R⁷ is 2,2-dioxido-2-thiaspiro[3.3]heptan-6-yl, 3,3-dioxido-3-thiabicyclo[3.1.0]hexan-6-yl, 2,2-dioxidohexahydro-1H-cyclopenta[c]thiophen-5-yl, or 2-imino-2-oxido-26-thiaspiro[3.3]heptan-6-yl.

194. In embodiment 194, the compound is as provided in any one of embodiments 1 to 121, or a pharmaceutically acceptable salt thereof, wherein R⁷ is a ring of formula (i) or (ii):

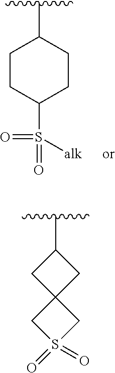

where alk is alkyl and each ring is substituted with R^{v} and R^{w}.

195. In embodiment 195, the compound is as provided in any one of embodiments 1 to 121, and 195, or a pharmaceutically acceptable salt thereof, wherein R⁷ is a ring of formula (i):

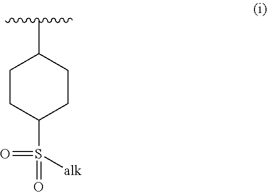

where alk is alkyl and the ring of formula (i) is substituted with R^{v} and R^{w}.

196. In embodiment 196, the compound is as provided in any one of embodiments 1 to 121, and 196, or a pharmaceutically acceptable salt thereof, wherein R⁷ is a ring of formula (i):

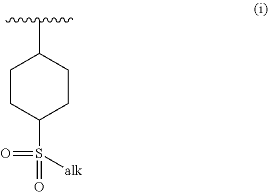

where alk is alkyl, R^{v} is hydrogen, and R^{w} is fluoro or methyl which is attached at carbon that is meta to the carbon substituted with —SO₂alk.

197. In embodiment 199, the compound is as provided in any one of embodiments 1 to 121, and 194, or a pharmaceutically acceptable salt thereof, wherein R⁷ is a ring of formula (I):

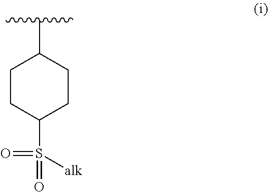

where alk is alkyl and R⁴ and R⁵ are hydrogen.

198. In embodiment 198, the compound is as provided in any one of embodiments 1 to 121, and 194, or a pharmaceutically acceptable salt thereof, wherein R⁷ is a ring of formula:

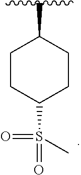

199. In embodiment 199, the compound is as provided in any one of embodiments 1 to 121, and 194, or a pharmaceutically acceptable salt thereof, wherein R⁷ is a ring of formula (ii):

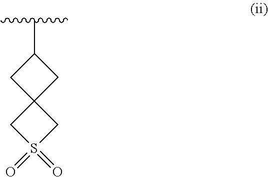 [SMILES: *C1CC2(C1)CS(=O)(=O)C2]

where the ring of formula (ii) is substituted with R^{v} and R^{w}.

200. In embodiment 200, the compound is as provided in any one of embodiments 1 to 121, and 194, or a pharmaceutically acceptable salt thereof, wherein R⁷ is a ring of formula:

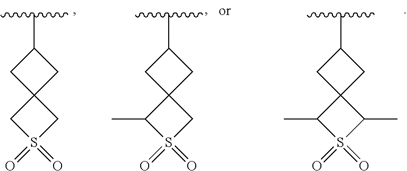

201. In embodiment 201, the compound is as provided in any one of embodiments 1 to 121, or a pharmaceutically acceptable salt thereof, wherein R⁷ is a ring of formula (iii) or (iv):

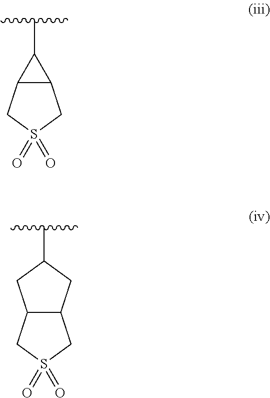 [SMILES: *C1C2CS(=O)(=O)CC12 ; *C1CC2CS(=O)(=O)CC2C1]

where each ring is substituted with R^{v} and R^{w}.

202. In embodiment 202, the compound is as provided in any one of embodiments 1 to 121, and 201, or a pharmaceutically acceptable salt thereof, wherein R⁷ is a ring of formula:

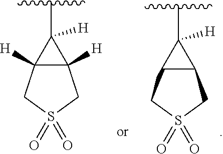

203. In embodiment 203, the compound is as provided in any one of embodiments 1 to 121, or a pharmaceutically acceptable salt thereof, wherein R⁷ is a ring of formula:

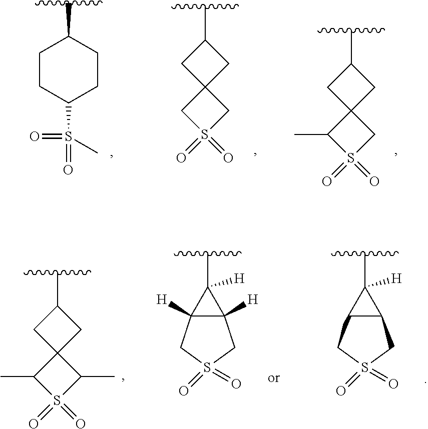

204. In embodiment 204, a compound disclosed in Table 1.

It is understood that the embodiments set forth above include all combinations of embodiments listed therein.

Additional embodiments include embodiments 1A to 84A:

1A. In embodiment 1A, is provided a compound of Formula (I) as defined in the first aspect.

2A. In embodiment 2A, the compound is as provided in embodiment 1A, or a pharmaceutically acceptable salt thereof, wherein:

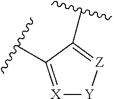

is

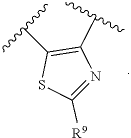

3A. In embodiment 3A, the compound is as provided in embodiment 1A or 2A, or a pharmaceutically acceptable salt thereof, having a structure according to formula (Ia) and (Ia′), respectively:

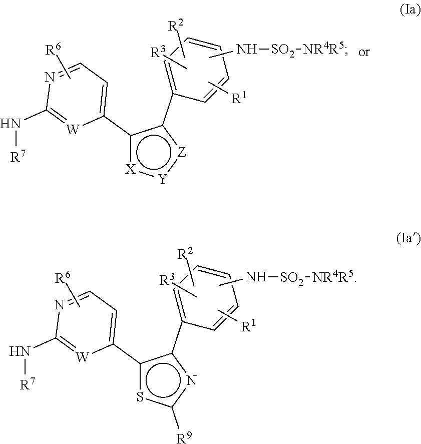

4A. In embodiment 4A, the compound is as provided in any one of embodiments 1A to 3A, or a pharmaceutically acceptable salt thereof, having a structure according to formula (Id) and (Id′), respectively:

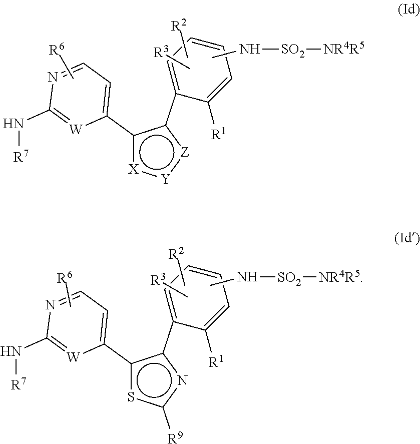

5A. In embodiment 5A, the compound is as provided in any one of embodiments 1A to 4A, or a pharmaceutically acceptable salt thereof, wherein R¹, R², and R³ are independently hydrogen, deuterium, alkyl, halo, haloalkyl, haloalkoxy, alkoxy, hydroxy, or cyano.

6A. In embodiment 6A, the compound is as provided in any one of embodiments 1A to 5A, or a pharmaceutically acceptable salt thereof, wherein R¹, R², and R³ are independently hydrogen, deuterium, methyl, ethyl, propyl, fluoro, chloro, bromo, difluoromethyl, trifluoromethyl, difluoromethoxy, trifluoromethoxy, methoxy, ethoxy, hydroxy, or cyano.

7A. In embodiment 7A, the compound is as provided in any one of embodiments 1A to 6A, or a pharmaceutically acceptable salt thereof, wherein R¹ and R² are independently hydrogen, deuterium, fluoro, chloro, difluoromethyl, trifluoromethyl, difluoromethoxy, trifluoromethoxy, methoxy, hydroxy, or cyano and R³ is hydrogen.

8A. In embodiment 8A, the compound is as provided in any one of embodiments 1A to 7A, or a pharmaceutically acceptable salt thereof, wherein R¹ is fluoro, R² is hydrogen, deuterium, fluoro, chloro, difluoromethyl, trifluoromethyl, difluoromethoxy, trifluoromethoxy, or cyano, and R³ is hydrogen.

9A. In embodiment 9A, the compound is as provided in any one of embodiments 1A to 8A, or a pharmaceutically acceptable salt thereof, wherein R¹ is fluoro and R² and R³ are hydrogen.

10A. In embodiment 1A, the compound is as provided in any one of embodiments 1A to 9A, or a pharmaceutically acceptable salt thereof, wherein R⁴ and R⁵ together with the nitrogen atom to which they are attached form cyclylaminyl, bicyclylaminyl, fused cyclylaminyl, bridged cyclylaminyl, or spiroaminyl, wherein cyclylaminyl, bicyclylaminyl, fused cyclylaminyl, bridged cyclylaminyl, and spiroaminyl are substituted with R^{s}, R^{t}, and R^{u}.

11A. In embodiment 11A, the compound is as provided in any one of embodiments 1A to 10A, or a pharmaceutically acceptable salt thereof, wherein R⁴ and R⁵ together with the nitrogen atom to which they are attached form fused cyclylaminyl substituted with R^{s}, R^{t}, and R^{u}.

12A. In embodiment 12A, the compound is as provided in any one of embodiments 1A to 11A, or a pharmaceutically acceptable salt thereof, wherein fused cyclylaminyl, formed by R⁴ and R⁵ together with the nitrogen atom to which they are attached, has a structure according to formula (a):

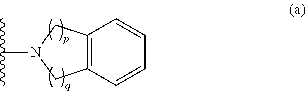

wherein:
p is 0, 1, or 2; and q is 0, 1, 2, 3, or 4; provided p+q is at least 2 and p+q is not more than 5; and when p or q is 3 or 4, then one of the —CH₂— of such p or q can be replaced NH, N (when attached to one of R^{s}, R^{t}, and R^{u}), O or S(O)ₙ; wherein n is 0, 1, or 2; and
the structure of formula (a) is substituted with R^{s}, R^{t}, and R^{u}.

13A. In embodiment 13A, the compound is as provided in any one of embodiments 1A to 11A, or a pharmaceutically acceptable salt thereof, wherein the fused cyclylaminyl, formed by R⁴ and R⁵ together with the nitrogen atom to which they are attached, has a structure according to formula (b):

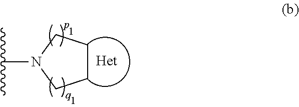

wherein:
p₁ is 0, 1, or 2; and q₁ is 0, 1, 2, 3, or 4; provided p+q is at least 2 and p+q is not more than 5; and when p or q is 3 or 4, then one of the —CH₂— of such p or q can be replaced by NH, N (when attached to one of R^{s}, R^{t}, and R^{u}), O or S(O)ₙ; wherein n is 0, 1, or 2;
Het is 5- or 6-membered heteroaryl; and
the structure of formula (b) is substituted with R^{s}, R^{t}, and R^{u}.

14A. In embodiment 14A, the compound is as provided in any one of embodiments 1A to 13A, or a pharmaceutically acceptable salt thereof, wherein R⁴ and R⁵ together with the nitrogen atom to which they are attached form a ring selected form:

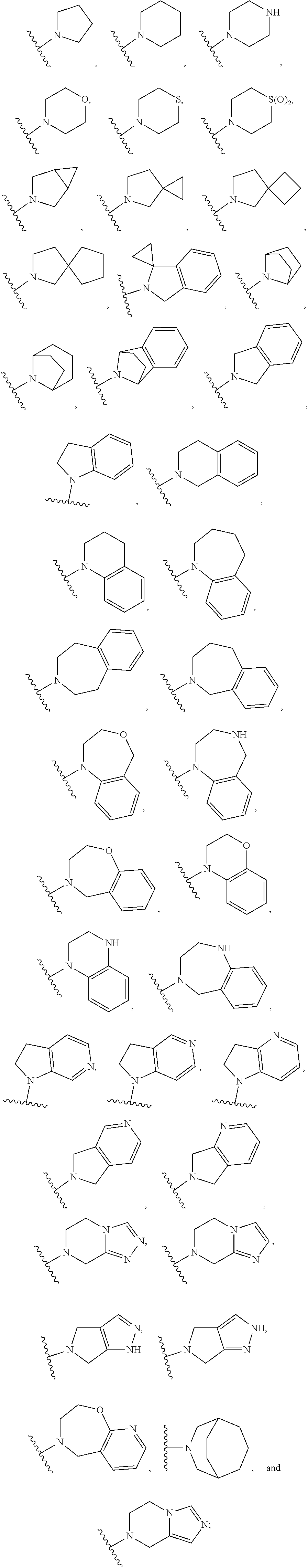

wherein each ring is substituted with R^{s}, R^{t}, and R^{u}.

15A. In embodiment 15A, the compound is as provided in any one of embodiments 1A to 14A, or a pharmaceutically acceptable salt thereof, wherein fused cyclylaminyl, formed by R⁴ and R⁵ together with the nitrogen atom to which they are attached, is selected from:

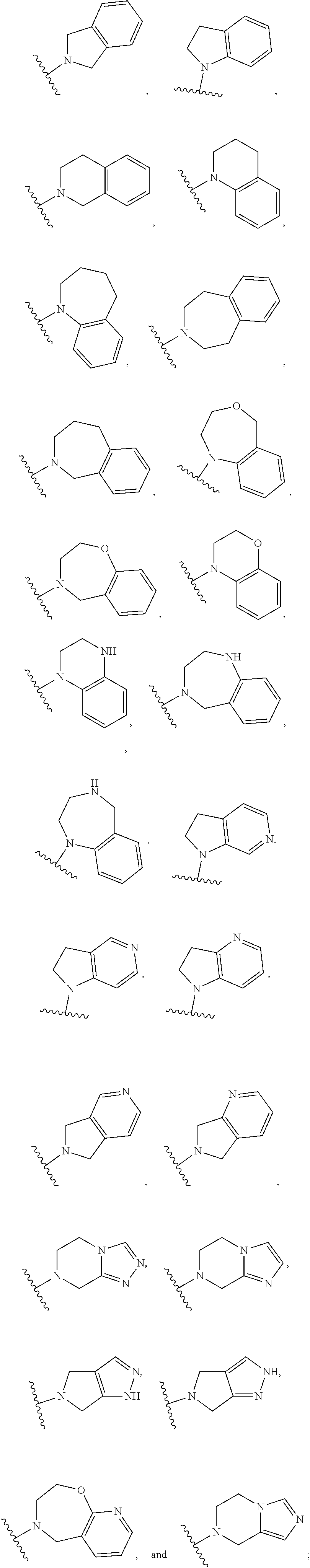

wherein each ring substituted with R^{s}, R^{t}, and R^{u}.

16A. In embodiment 16A, the compound is as provided in any one of embodiments 1A to 15A, or a pharmaceutically acceptable salt thereof, wherein fused cyclylaminyl, formed by R⁴ and R⁵ together with the nitrogen atom to which they are attached, is selected from:

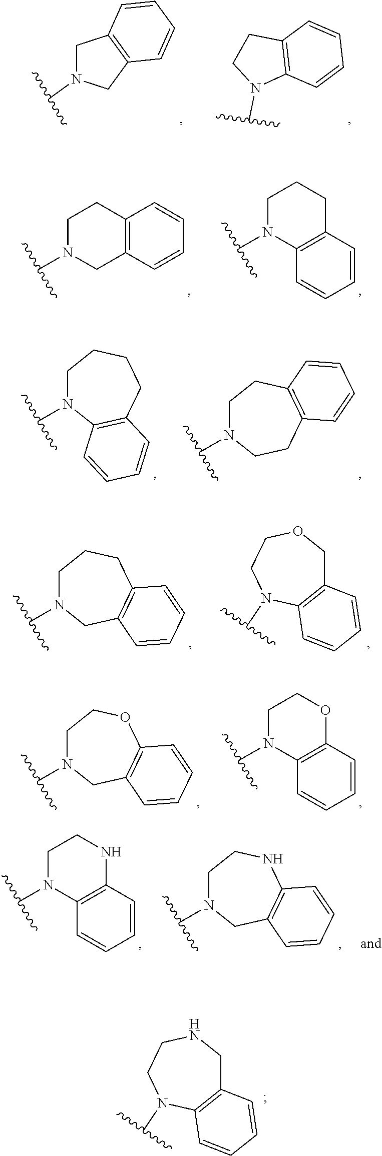

wherein each ring is substituted with R^{s}, R^{t}, and R^{u}.

17A. In embodiment 17A, the compound is as provided in any one of embodiments 1A to 15A, or a pharmaceutically acceptable salt thereof, wherein fused cyclylaminyl, formed by R⁴ and R⁵ together with the nitrogen atom to which they are attached, is selected from:

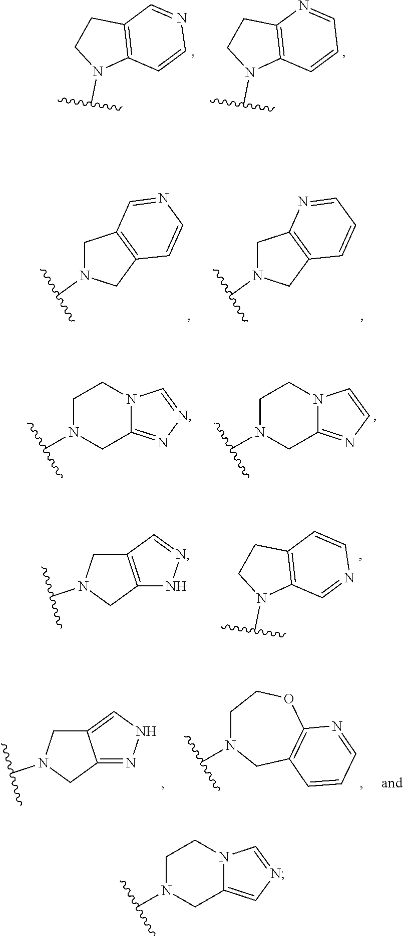

wherein each ring substituted with R^{s}, R^{t}, and R^{u}.

18A. In embodiment 18A, the compound is as provided in any one of embodiments 1A to 12A and 14A to 16A, or a pharmaceutically acceptable salt thereof, wherein the fused cyclylaminyl, formed by R⁴ and R⁵ together with the nitrogen atom to which they are attached, is a ring of formula (a1) or (a2):

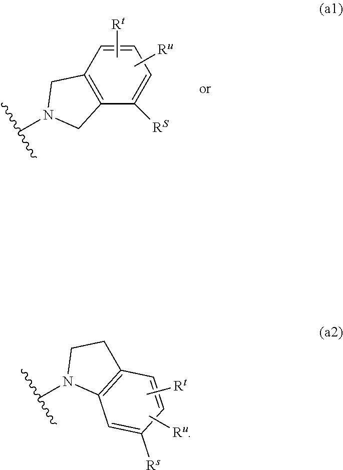

19A. In embodiment 19A, the compound is as provided in any one of embodiments 1A to 18A, or a pharmaceutically acceptable salt thereof, wherein R^{s} is hydrogen, deuterium, alkyl, alkoxy, alkylsulfonyl, hydroxy, acyl, halo, haloalkyl, haloalkoxy, amino, alkylamino, dialkylamino, substituted amino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, or unsaturated heterocyclyl (wherein aryl, by itself or as part of aryloxy and aralkyl, heteroaryl, by itself or as part of heteroaryloxy and heteroaralkyl heterocyclyl, by itself or as part of heterocyclylalkyl and heterocyclyloxy and unsaturated heterocyclyl are substituted with one to three substituents independently selected from hydrogen, alkyl, alkoxy, hydroxy, halo, haloalkyl, haloalkoxy, and cyano), R^{t} is hydrogen, alkyl, alkoxy, alkylsulfonyl, hydroxy, acyl, halo, haloalkyl, haloalkoxy, amino, alkylamino, dialkylamino, and cyano, and R^{u} is hydrogen.

20A. In embodiment 20A, the compound is as provided in any one of embodiments 1A to 19A, or a pharmaceutically acceptable salt thereof, wherein R^{s} is selected from hydrogen, deuterium, methyl, ethyl, isopropyl, cyclopropyl, cyclohexyl, methoxy, ethoxy, isopropoxy, methylsulfonyl, ethylsulfonyl, hydroxy, methylcarbonyl, fluoro, chloro, difluoromethyl, trifluoromethyl, difluoromethoxy, trifluoromethoxy, amino, methylamino, dimethylamino, cyano, cyanomethyl, 2-cyanoprop-2-yl, cyanomethyloxy, hydroxymethyl, 1-hydroxyethyl, 1-hydroxy-1-methylethyl, methoxymethyl, methylaminomethyl, dimethylaminomethyl, methoxycarbonylaminomethyl, —CONH₂, methylaminocarbonyl, dimethylaminocarbonyl, 2-hydroxyethloxy, —O—(CH₂)₂NH₂, 2-methylaminoethyloxy, 2-dimethylaminoethyloxy, phenyl, phenoxy, 3-fluorophenoxy, 4-fluorophenoxy, 2-cyanophenoxy, 3-cyanophenyl, benzyl, 1-methyl-2-oxo-1,6-dihydropyridinyl, and morpholin-4-ylmethyl, R^{t} is hydrogen, fluoro, chloro, methoxy, difluoromethyl, difluoromethoxy, trifluoromethyl, trifluoromethoxy, or cyano, and R^{u} is hydrogen.

21A. In embodiment 21A, the compound is as provided in any one of embodiments 1A to 20A, or a pharmaceutically acceptable salt thereof, wherein R⁴ and R⁵ together with the nitrogen atom to which they are attached form a ring selected from morpholin-4-yl, piperidin-1-yl, 2-methylpiperidin-1-yl, 3-methylpiperidin-1-yl, 4-methylpiperidin-1-yl, 4-phenoxypiperidin-1-yl, 2-phenylpiperidin-1-yl, 2-phenylpyrrolidin-1-yl, 3,3-difluoropyrrolidin-1-yl, 4-phenylpiperazin-1-yl, 4-acetylpiperazin-1-yl, 4-methyl-3-oxo-piperazin-1-yl, 4-methylsulfonylpiperazin-1-yl, 3-azabicyclo[3.1.0]hexan-3-yl, azaspiro[2.4]heptan-5-yl, 2-azaspiro[4.4]nonan-2-yl, 6-azaspiro[3.4]octan-6-yl, S-azaspiro[2.4]heptan-5-yl, 7-azabicyclo[2.2.1]heptan-7-yl, 8-azabicyclo[3.2.1]octan-8-yl, 2-azabicyclo[2.2.2]octan-2-yl, 3-azabicyclo[3.2.2]nonan-3-yl, isoindolin-2-yl, 4-cyanoisoindolin-2-yl, 5-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, 5-hydroxymethylisoindolin-2-yl, 4-difluoromethylisoindolin-2-yl, S-difluoromethylisoindolin-2-yl, 4-difluoromethoxyisoindolin-2-yl, 4-trifluoromethylisoindolin-2-yl, S-trifluoromethylisoindolin-2-yl, 4-fluoroisoindolin-2-yl, 5-fluoroisoindolin-2-yl, 5,6-difluoroisoindolin-2-yl, 1-methylisoindoline-2-yl, 4-methylaminocarbonylisoindolin-2-yl, 4-dimethylaminocarbonyl-isoindolin-2-yl, 5-dimethylaminocarbonylisoindolin-2-yl, 4-(2-(dimethylamino)ethoxy)-isoindolin-2-yl, 4-(2-(methylamino)ethoxy)isoindolin-2-yl, 4-dimethylaminomethylisoindolin-2-yl, 5-dimethylaminomethylisoindolin-2-yl, 4-methylaminomethylisoindolin-2-yl, 4-dimethylaminoisoindolin-2-yl, 4-methylaminoisoindolin-2-yl, 3-phenylisoindolin-2-yl, 4-phenylisoindolin-2-yl, 4-phenoxyisoindolin-2-yl, 1-benzylisoindolin-2-yl, 4-benzylisoindolin-2-yl, 4-(3-fluorophenoxy)isoindolin-2-yl, 4-(4-fluorophenoxy)isoindolin-2-yl, 4-(2-cyanophenoxy)-isoindolin-2-yl, 4-(3-cyanophenoxy)-isoindolin-2-yl, 4-cyclohexylisoindolin-2-yl, 5-(hydroxymethyl)isoindolin-2-yl, 4-(2-hydroxy-ethoxy)isoindolin-2-yl, 4-(2-aminoethoxy)-isoindolin-2-yl, 5-(cyanomethyl)isoindolin-2-yl, 4-(cyanomethyl)isoindolin-2-yl, indolin-1-yl, 2-methylindolin-1-yl, 4-cyanoindolin-1-yl, 5-cyanoindolin-1-yl, 6-cyanoindolin-1-yl, 4-hydroxy-methylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloro-indolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxy-indolin-1-yl, 6-trifluoromethoxylindolin-1-yl, 4-((dimethylamino)-methyl)indolin-1-yl, 5-((dimethyl-amino)methyl)indolin-1-yl, 6-((dimethylamino)methyl)indolin-1-yl, 4-((methylamino)-methyl)indolin-1-yl, 5-((methylamino)methyl)indolin-1-yl, 6-((methylamino)-methyl)indolin-1-yl, 6-methylaminocarbonylindolin-1-yl, 6-aminocarbonyl-indolin-1-yl, 6-dimethylamino-carbonylindolin-1-yl, 4-(2-(methylamino)ethoxy) indolin-1-yl, 6-(2-(dimethylamino)ethoxy)-indolin-1-yl, 4-(hydroxymethyl)indolin-1-yl, 5-phenylindolin-1-yl, 6-(1-methyl-6-oxo-1,6-dihydropyridin-2-yl)indolin-1-yl, 4-(1-hydroxyethyl)indolin-1-yl, 4-(2-hydroxypropan-2-yl)-indolin-1-yl, 6-(2-hydroxypropan-2-yl)indolin-1-yl, 4-(cyanomethyl)indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, 6-chloroindolin-1-yl, 3,4-dihydroisoquinolin-2(1H)-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepin-2-yl, 1-methyl-1,2,3,5-tetrahydro-4H-benzo[e][1,4]diazepin-4-yl, and 2,3-dihydrobenzo[e][1,4]oxazepin-1(5H)-yl.

22A. In embodiment 22A, the compound is as provided in any one of embodiments 1A to 21A, or a pharmaceutically acceptable salt thereof, wherein R⁴ and R⁵ together with the nitrogen atom to which they are attached form a ring selected from isoindolin-2-yl, 4-cyanoisoindolin-2-yl, 5-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, S-hydroxymethylisoindolin-2-yl, 4-difluoromethylisoindolin-2-yl, 5difluoromethylisoindolin-2-yl, 4-difluoromethoxyisoindolin-2-yl, 4-trifluoromethylisoindolin-2-yl, 5-trifluoromethylisoindolin-2-yl, 4-fluoroisoindolin-2-yl, 5-fluoroisoindolin-2-yl, 5,6-difluoroisoindolin-2-yl, 1-methylisoindoline-2-yl, 4-methylamino-carbonylisoindolin-2-yl, 4-dimethylaminocarbonyl-isoindolin-2-yl, 5-dimethylaminocarbonyl-isoindolin-2-yl, 4-(2-(dimethylamino)ethoxy)-isoindolin-2-yl, 4-(2-(methylamino)ethoxy)-isoindolin-2-yl, 4-dimethylaminomethylisoindolin-2-yl, 5-dimethylaminomethylisoindolin-2-yl, 4-methylaminomethylisoindolin-2-yl, 4-dimethyl-aminoisoindolin-2-yl, 4-methylaminoisoindolin-2-yl, 3-phenylisoindolin-2-yl, 4-phenylisoindolin-2-yl, 4-phenoxyisoindolin-2-yl, 1-benzylisoindolin-2-yl, 4-benzylisoindolin-2-yl, 4-(3-fluoro-phenoxy)isoindolin-2-yl, 4-(4-fluorophenoxy)-isoindolin-2-yl, 4-(2-cyanophenoxy)isoindolin-2-yl, 4-(3-cyanophenoxy)-isoindolin-2-yl, 4-cyclohexylisoindolin-2-yl, 5-(hydroxymethyl)isoindolin-2-yl, 4-(2-hydroxyethoxy)isoindolin-2-yl, 4-(2-aminoethoxy)isoindolin-2-yl, 5-(cyanomethyl)-isoindolin-2-yl, 4-(cyanomethyl)isoindolin-2-yl, indolin-1-yl, 2-methylindolin-1-yl, 4-cyanoindolin-1-yl, 5-cyanoindolin-1-yl, 6-cyanoindolin-1-yl, 4-hydroxymethylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloroindolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxyindolin-1-yl, 6-trifluoromethoxylindolin-1-yl, 4-((dimethylamino)methyl)-indolin-1-yl, 5 ((dimethyl-amino)methyl)indolin-1-yl, 6-((dimethyl-amino)methyl)indolin-1-yl, 4-((methylamino)-methyl)indolin-1-yl, 5-((methylamino)methyl)-indolin-1-yl, 6-((methylamino)-methyl)indolin-1-yl, 6-methylaminocarbonylindolin-1-yl, 6-aminocarbonyl-indolin-1-yl, 6-dimethylamino-carbonylindolin-1-yl, 4-(2-(methylamino)ethoxy)-indolin-1-yl, 6-(2-(dimethylamino)ethoxy)-indolin-1-yl, 4-(hydroxymethyl)indolin-1-yl, 5-phenylindolin-1-yl, 6-(1-methyl-6-oxo-1,6-dihydropyridin-2-yl)indolin-1-yl, 4-(1-hydroxyethyl)-indolin-1-yl, 4-(2-hydroxypropan-2-yl)indolin-1-yl, 6-(2-hydroxypropan-2-yl)indolin-1-yl, 4-(cyanomethyl)-indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, 6-chloroindolin-1-yl, 3,4-dihydroisoquinolin-2-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepine-2-yl, 1-methyl-1,2,3,5-tetrahydro-4H-benzo[e][1,4]diazepin-4-yl, and 2,3-dihydrobenzo[e][1,4]oxazepin-1(5H)-yl.

23A. In embodiment 23A, the compound is as provided in any one of embodiments 1A to 22A, or a pharmaceutically acceptable salt thereof, wherein W is N.

24A. In embodiment 24A, the compound is as provided in any one of embodiments 1A to 23A, or a pharmaceutically acceptable salt thereof, wherein R⁹ and R¹⁰ are independently alkyl, cycloalkyl, bridged cycloalkyl, spiro cycloalkyl, heterocyclyl, bridged heterocyclyl, or spiro heterocyclyl, wherein:
(A) cycloalkyl, bridged cycloalkyl, and spiro cycloalkyl of R⁹ and R¹⁰ are substituted with one or two R^{a}; and
(B) heterocyclyl, bridged heterocyclyl, and spiro heterocyclyl of R⁹ and R¹⁰ are substituted with R^{b}, R^{c}, and R^{d}.

25A. In embodiment 25A, the compound is as provided in any one of embodiments 1A to 24A, or a pharmaceutically acceptable salt thereof, wherein R⁹ and R¹⁰ are alkyl.

26A. In embodiment 26A, the compound is as provided in any one of embodiments 1A to 25A, or a pharmaceutically acceptable salt thereof, wherein R⁹ and R¹⁰ are methyl, ethyl, propyl, or butyl.

27A. In embodiment 27A, the compound is as provided in any one of embodiments 1A to 26A, or a pharmaceutically acceptable salt thereof, wherein R⁹ and R¹⁰ are isopropyl, iso-butyl, sec-butyl, or tert-butyl.

28A. In embodiment 28A, the compound is as provided in any one of embodiments 1A to 24A, or a pharmaceutically acceptable salt thereof, wherein R⁹ and R¹⁰ are independently cycloalkyl, bridged cycloalkyl, or spiro cycloalkyl wherein the cycloalkyl, bridged cycloalkyl, and spiro cycloalkyl of R⁹ and R¹⁰ are substituted with one or two R^{a}.

29A. In embodiment 29A, the compound is as provided in any one of embodiments LA to 24A and 27A to 28A, or a pharmaceutically acceptable salt thereof, wherein the one or two R^{a} are independently selected from hydrogen, deuterium, methyl, trideuteromethyl, fluoro, chloro, difluoromethyl, trifluoromethyl, and cyano.

30A. In embodiment 30A, the compound is as provided in any one of embodiments 1A to 24A and 27A to 29A, or a pharmaceutically acceptable salt thereof, wherein R⁹ and R¹⁰ are independently cycloalkyl and bridged cycloalkyl, each ring substituted with one or two R^{a}.

31A. In embodiment 31A, the compound is as provided in any one of embodiments 1A to 24A and 27A to 30A, or a pharmaceutically acceptable salt thereof, wherein R⁹ and R¹⁰ are cycloalkyl substituted with one or two R^{a}.

32A. In embodiment 32A, the compound is as provided in any one of embodiments 1A to 24A and 27A to 30A, or a pharmaceutically acceptable salt thereof, wherein R⁹ and R¹⁰ are bridged cycloalkyl substituted with one or two R^{a}.

33A. In embodiment 33A, the compound is as provided in any one of embodiments 1A to 24A, or a pharmaceutically acceptable salt thereof, wherein R⁹ and R¹⁰ are independently heterocyclyl, bridged heterocyclyl, or spiro heterocyclyl, wherein the heterocyclyl, bridged heterocyclyl, and spiro heterocyclyl of R⁹ and R¹⁰ are substituted with R^{b}, R^{c}, and R^{d}.

34A. In embodiment 34A, the compound is as provided in any one of embodiments 1A to 24A and 33A, or a pharmaceutically acceptable salt thereof, wherein R⁹ and R¹⁰ are independently heterocyclyl or bridged heterocyclyl, each substituted with R^{b}, R^{c}, and R^{d}.

35A. In embodiment 35A, the compound is as provided in any one of embodiments 1A to 24A, 33A, and 34A, or a pharmaceutically acceptable salt thereof, wherein R⁹ and R¹⁰ are heterocyclyl substituted with R^{b}, R^{c}, and R^{d}.

36A. In embodiment 36A, the compound is as provided in any one of embodiments 1A to 24A, 33A, and 34A, or a pharmaceutically acceptable salt thereof, wherein R⁹ and R¹⁰ are bridged heterocyclyl substituted with R^{b}, R^{c}, and R^{d}.

37A. In embodiment 37A, the compound is as provided in any one of embodiments 1A to 24A, 28A to 35A, or a pharmaceutically acceptable salt thereof, wherein cycloalkyl and bridged cycloalkyl of R⁹ and R¹⁰ are selected from:

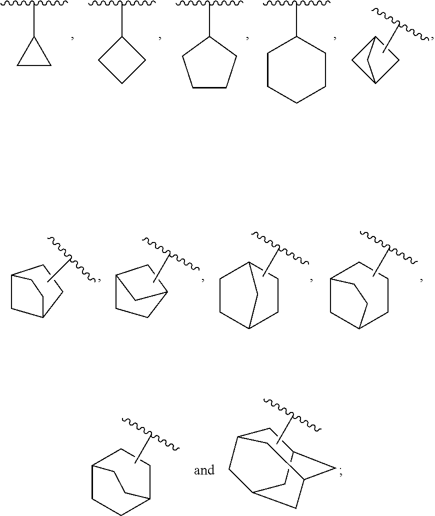

and heterocyclyl and bridged heterocyclyl of R⁹ and R¹⁰ are selected from:

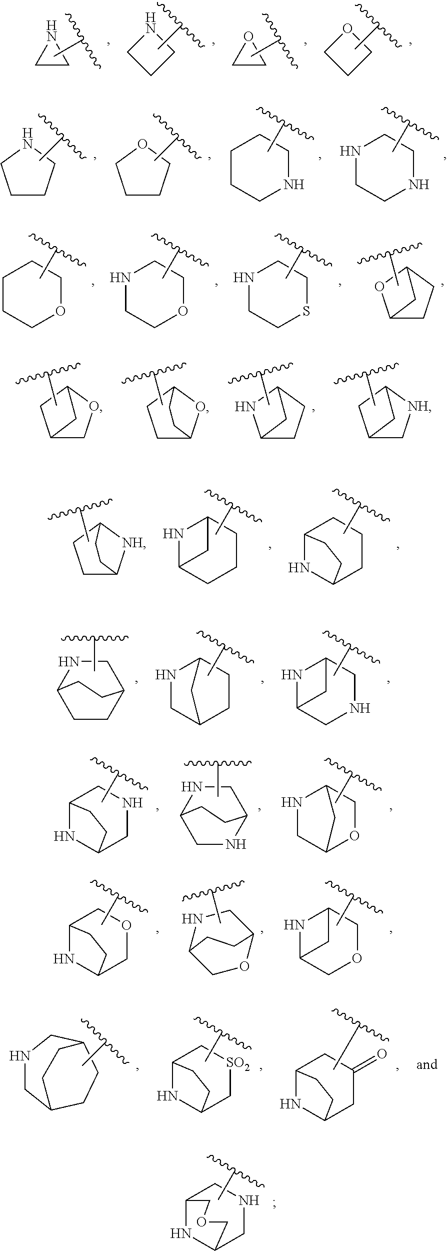

wherein each cycloalkyl and bridged cycloalkyl is substituted with R^{a}; and each heterocyclyl and bridged heterocyclyl is substituted with R^{b}, R^{c}, and R^{d}.

38A. In embodiment 38A, the compound is as provided in any one of embodiments 1A to 24A, 28A to 31A, and 37A, or a pharmaceutically acceptable salt thereof, wherein the cycloalkyl of ring R⁹ and R¹⁰ are selected from:

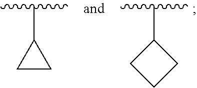

wherein each cycloalkyl is substituted with one or two R^{a}.

39A. In embodiment 39A, the compound is as provided in any one of embodiments 1A to 24A, 28A to 30A, 32A, and 37A, or a pharmaceutically acceptable salt thereof, wherein, wherein the bridged cycloalkyl of R⁹ and R¹⁰ are selected from:

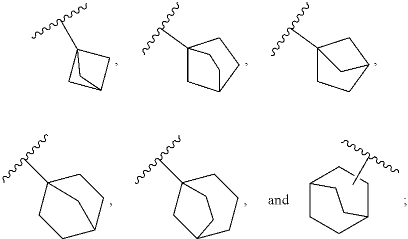

wherein each bridged cycloalkyl is substituted with R^{a}.

40A. In embodiment 40A, the compound is as provided in any one of embodiments 1A to 24A, 29A, 33A, 34A, 35A, and 37A, or a pharmaceutically acceptable salt thereof, wherein the heterocyclyl of R⁹ and R¹⁰ is selected from:

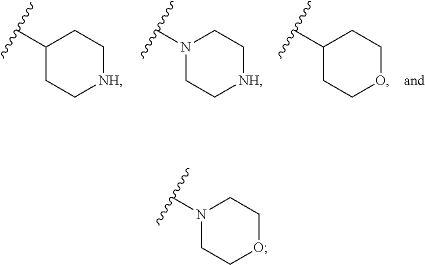 [SMILES: *C1CCNCC1 ; *N1CCNCC1 ; *C1CCOCC1 ; "and" ; *N1CCOCC1]

wherein each heterocyclyl is substituted with R^{b}, R^{c}, and R^{d}.

41A. In embodiment 41A, the compound is as provided in any one of embodiments 1A to 24A, 29A, 33A, 34A, 36A, and 37A, or a pharmaceutically acceptable salt thereof, wherein the bridged heterocyclyl of R⁹ and R¹⁰ is selected from:

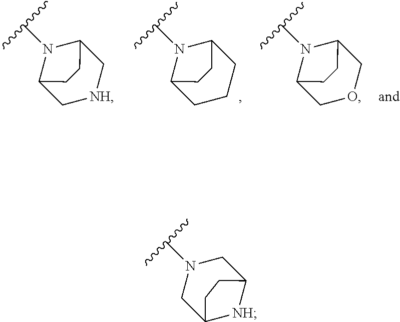 [SMILES: *N1C2CCC1CNC2 ; *N1C2CCCC1CC2 ; *N1C2CCC1COC2 ; "and" ; *N1CC2CCC(C1)N2]

wherein each bridged heterocyclyl is substituted with R^{b}, R^{c}, and R^{d}.

42A. In embodiment 42A, the compound is as provided in any one of embodiments 1A to 24A, 28A to 32A, and 37A to 39A, or a pharmaceutically acceptable salt thereof, wherein R^{a} is hydrogen, deuterium, chloro, fluoro, difluoromethyl, or trifluoromethyl.

43A. In embodiment 43A, the compound is as provided in any one of embodiments 1A to 24A, 28A to 32A, 37A to 39A, and 42A, or a pharmaceutically acceptable salt thereof, wherein bridged cycloalkyl of R⁹ and R¹⁰ is bicyclo[1.1.1]pentan-1-yl, 3-fluorobicyclo[1.1.1]pentan-1-yl, 3-chlorobicyclo-[1.1.1]pentan-1-yl, 3-(hydroxymethyl)-bicyclo[1.1.1]pentan-1-yl, 3-(trifluoromethyl)-bicyclo[1.1.1]pentan-1-yl, 3-(difluoromethyl)-bicyclo[1.1.1]pentan-1-yl, and cycloalkyl of R⁹ and R¹⁰ is cyclobutyl, cyclopropyl, 1-(difluoromethyl)cyclobutyl, 1-(trifluoromethyl)cyclobutyl, 1-(difluoromethyl)cyclopropyl, or 1-(trifluoromethyl)cyclopropyl.

44A. In embodiment 44A, the compound is as provided in any one of embodiments 1A to 24A, 29A, and 33A to 43A, or a pharmaceutically acceptable salt thereof, wherein R^{b} and R^{c} are independently selected from hydrogen, methyl, fluoro, chloro, difluoromethyl, trifluoromethyl, 2,2-difluoroethyl, 2,2,2-trifluoroethyl, 3,3,3-trifluoropropyl, hydroxy, and cyano, and R^{d} is selected from hydrogen, methyl, trideuteromethyl, cyclopropyl, cyclobutyl, cyclopentyl, cyclohexyl, 2,2-difluoroethyl, 2,2,2-trifluoroethyl, 3,3,3-trifluoropropyl, amino, dimethylamino, diethylamino, 3,3-difluorocyclobutyl, 4,4-difluorocyclohexyl, 3-hydroxy-3-methylcyclobutyl, 3-cyano-3-methylcyclobutyl, oxetan-3-yl, tetrahydrofuran-2-yl, tetrahydropyran-4-yl, 1,1-dioxidothietan-3-yl, 1,1-dioxidotetrahydro-2H-thiopyran-4-yl, benzyl, phenyl, pyridin-2-yl, pyridin-3-yl, and pyridin-4-yl.

45A. In embodiment 45A, the compound is as provided in any one of embodiments 1A to 24A, 29A, 33A to 44A, or a pharmaceutically acceptable salt thereof, wherein R^{b} is hydrogen, R^{c} is selected from hydrogen, methyl, fluoro, chloro, difluoromethyl, trifluoromethyl, 2,2-difluoroethyl, 2,2,2-trifluoroethyl, 3,3,3-trifluoropropyl, hydroxy, and cyano, and R^{d} is selected from hydrogen, methyl, trideuteromethyl, cyclopropyl, cyclobutyl, cyclopentyl, cyclohexyl, 2,2-difluoroethyl, 2,2,2-trifluoroethyl, 3,3,3-trifluoropropyl, amino, dimethylamino, diethylamino, 3,3-difluorocyclobutyl, 4,4-difluorocyclohexyl, 3-hydroxy-3-methylcyclobutyl, 3-cyano-3-methylcyclobutyl, oxetan-3-yl, tetrahydrofuran-2-yl, tetrahydropyran-4-yl, 1,1-dioxidothietan-3-yl, 1,1-dioxidotetrahydro-2H-thiopyran-4-yl, benzyl, phenyl, pyridin-2-yl, pyridin-3-yl, and pyridin-4-yl.

46A. In embodiment 46A, the compound is as provided in any one of embodiments 1A to 45A, or a pharmaceutically acceptable salt thereof, wherein R⁷ is -Q-(alk¹)ₙ₁-SO₂R¹¹, -Q²-(alk³)ₙ₃-SO₂NR¹⁴R¹⁵, heterocyclyl, bicyclic heterocyclyl, spiro heterocyclyl, or spiro sulfoximinyl, wherein heterocyclyl, bicyclic heterocyclyl, spiro heterocyclyl, and spiro sulfoximinyl are substituted with R^{x}, R^{y}, and R^{y1}.

47A. In embodiment 47A, the compound is as provided in any one of embodiments 1A to 45A, or a pharmaceutically acceptable salt thereof, wherein R⁷ is aryl, heteroaryl, or fused heteroaryl, where each of the aforementioned ring is substituted with R^{x}, R^{y}, and R^{y1}.

48A. In embodiment 48A, the compound is as provided in any one of embodiments 1A to 46A, or a pharmaceutically acceptable salt thereof, wherein R⁷ is-Q-(alk¹)ₙ₁-SO₂R¹¹.

49A. In embodiment 49A, the compound is as provided in any one of embodiments 1A to 46A, or a pharmaceutically acceptable salt thereof, wherein R⁷ is -Q²-(alk³)ₙ₃-SO₂NR¹⁴R¹⁵.

49B. In embodiment 49B, the compound is as provided in any one of embodiments 1 to 46, or a pharmaceutically acceptable salt thereof, wherein R⁷ is heterocyclyl substituted with R^{x}, R^{y}, and R^{y1}.

50A. In embodiment 50A, the compound is as provided in any one of embodiments 1A to 46A, or a pharmaceutically acceptable salt thereof, wherein R⁷ is bicyclic heterocyclyl substituted with R^{x}, R^{y}, and R^{y1}.

51A. In embodiment 51A, the compound is as provided in any one of embodiments 1A to 46A, or a pharmaceutically acceptable salt thereof, wherein R⁷ is spiro heterocyclyl substituted with R^{x}, R^{y}, and R^{y1}.

52A. In embodiment 52A, the compound is as provided in any one of embodiments 1A to 46A, 48A, and 49A, or a pharmaceutically acceptable salt thereof, wherein each of Q, Q¹, Q², Q³, Q⁴, Q⁵, and Q⁶ is cycloalkyl, bridged cycloalkyl, spiro cycloalkyl, heterocyclyl^{A}, bicyclic heterocyclyl^{A}, bridged heterocyclyl^{A}, spiro heterocyclyl^{A}, aryl, or heteroaryl, where each of the aforementioned ring is substituted with R^{v} and R^{w}.

53A. In embodiment 53A, the compound is as provided in any one of embodiments 1A to 46A, 48A, 49A, and 52A, or a pharmaceutically acceptable salt thereof, wherein each of Q, Q¹, Q², Q³, Q⁴, Q⁵, and Q⁶ is cycloalkyl substituted with R^{v} and R^{w}.

54A. In embodiment 54A, the compound is as provided in any one of embodiments 1A to 46A, 48A, 49A, and 52A, or a pharmaceutically acceptable salt thereof, wherein each of Q, Q¹, Q², Q³, Q⁴, Q⁵, and Q⁶ is bridged cycloalkyl substituted with R^{v} and R^{w}.

55A. In embodiment 55A, the compound is as provided in any one of embodiments 1A to 46A, 48A, 49A, and 52A, or a pharmaceutically acceptable salt thereof, wherein each of Q, Q¹, Q², Q³, Q⁴, Q⁵, and Q⁶ is heterocyclyl^{A} substituted with R^{v} and R^{w}.

56A. In embodiment 56A, the compound is as provided in any one of embodiments 1A to 46A, 48A, 49A, and 52A, or a pharmaceutically acceptable salt thereof, wherein each of Q, Q¹, Q², Q³, Q⁴, Q⁵, and Q⁶ is bicyclic heterocyclyl^{A} substituted with R^{v} and R^{w}.

57A. In embodiment 57A, the compound is as provided in any one of embodiments 1A to 46A, 48A, 49A, and 52A, or a pharmaceutically acceptable salt thereof, wherein each of Q, Q¹, Q², Q³, Q⁴, Q⁵, and Q⁶ is aryl, or heteroaryl, where each of the aforementioned ring is substituted with R^{v} and R^{w}.

58A. In embodiment 58A, the compound is as provided in any one of embodiments 1A to 46A, 48A, 49A, and 52A to 57A, or a pharmaceutically acceptable salt thereof, wherein each of n¹, n², n³, n⁴, n⁵, n⁶, and n⁷ is 0.

59A. In embodiment 59A, the compound is as provided in any one of embodiments 1A to 58A, or a pharmaceutically acceptable salt thereof, wherein
(1) each of Q, Q¹, Q², Q³, Q⁴, Q⁵, and Q⁶ is independently selected from:

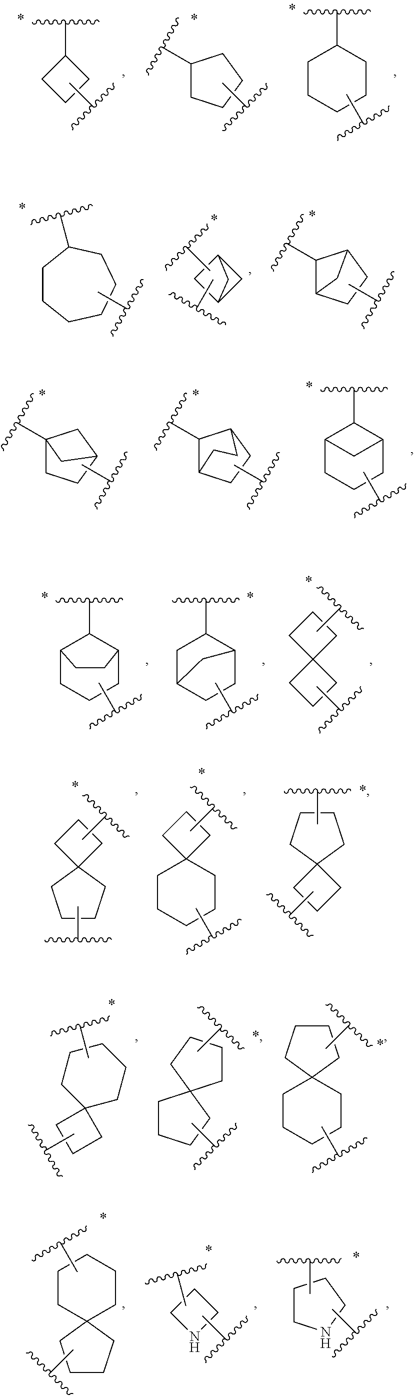

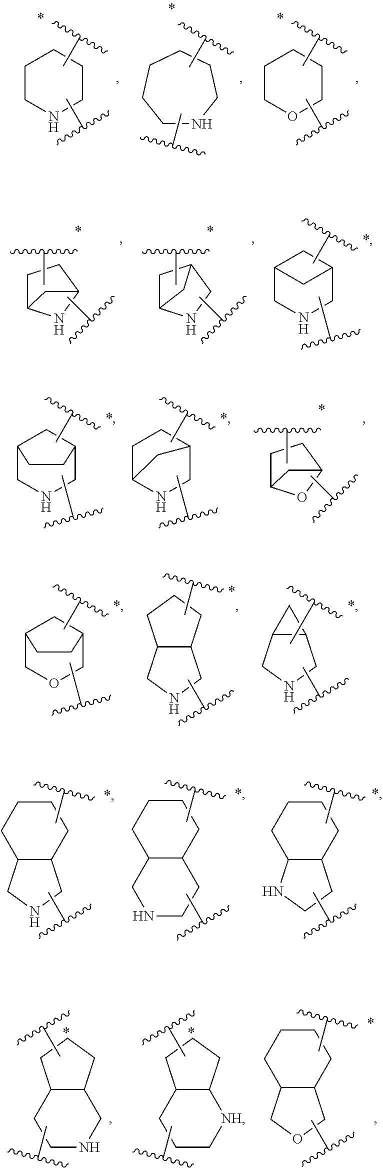

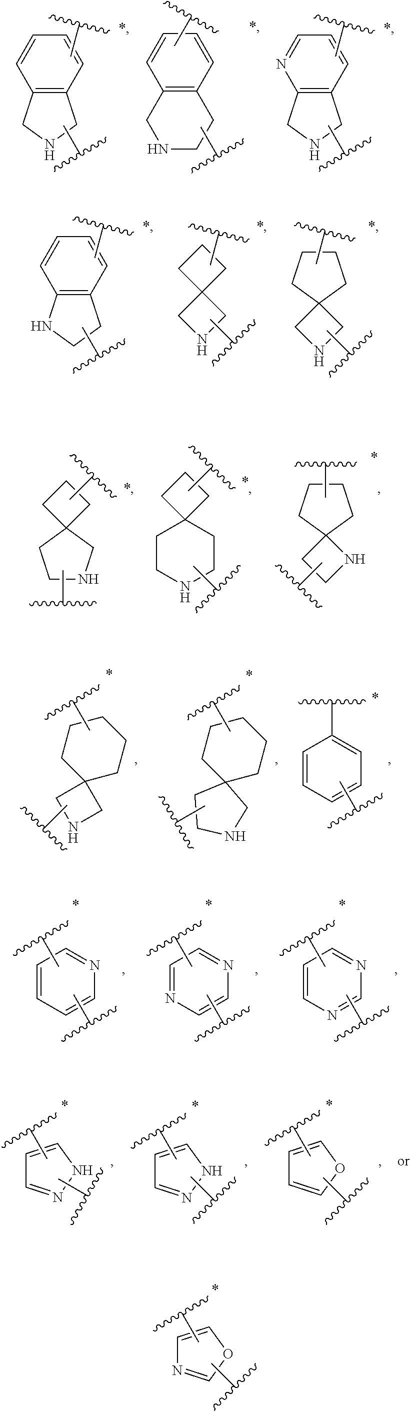

wherein each ring of (1) is substituted with R^{v} and R^{w};
and
(2). heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
   bridged heterocyclyl is:

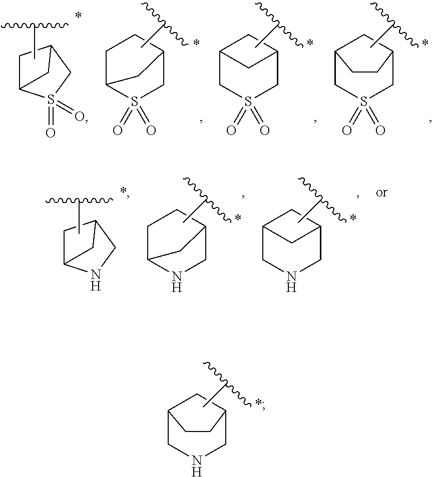

bicyclic heterocyclyl is:

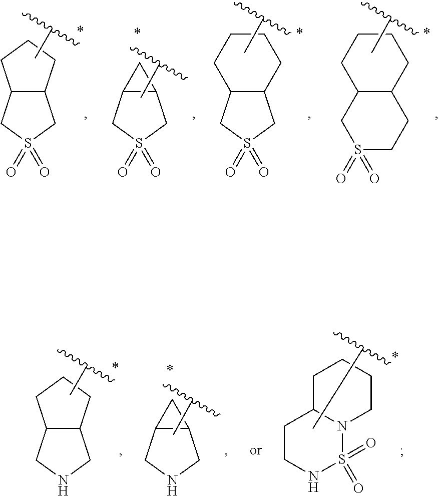

spiro heterocyclyl is:

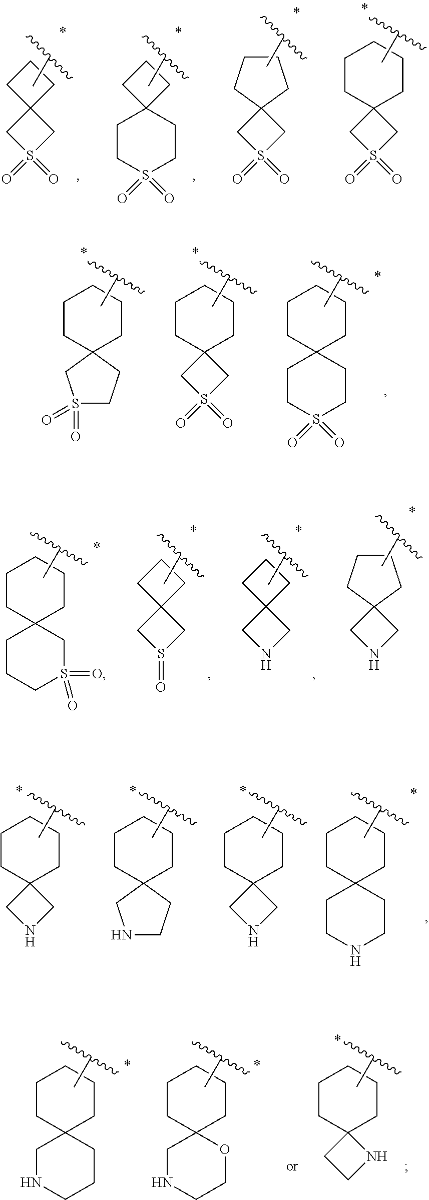

heterocyclvl is:

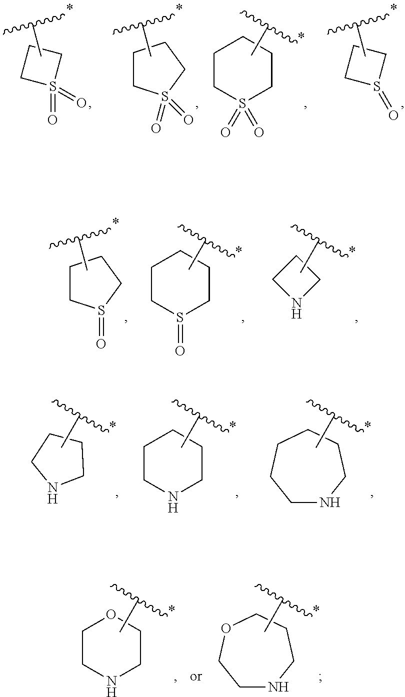

spiro sulfoximinyl is:

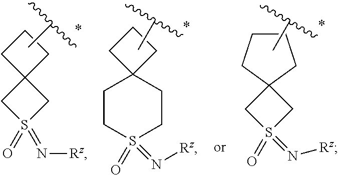

cyclylsulfoximinyl is:

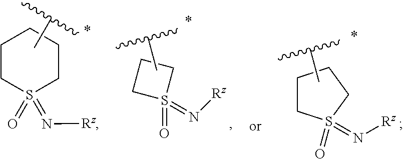

aryl is:

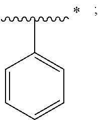 [SMILES: *c1ccccc1]

heteroaryl is:

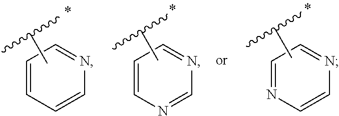

and
fused heteroaryl is:

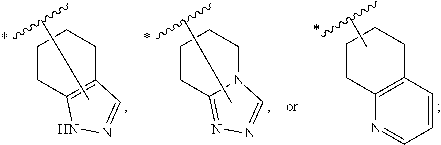

wherein each ring in (2) is substituted with R^{x}, R^{y}, and R^{y1}; and wherein

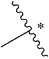

denotes the bond to NH of —NHR⁷ moiety of the compound of Formula (I); and

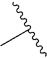

denotes bond to remainder -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, -Q⁶-(alk⁶)ₙ₇-NR²¹SO₂R²².

60A. In embodiment 60A, the compound is as provided in any one of embodiments 1A to 59A, or a pharmaceutically acceptable salt thereof, wherein R⁷ is where:
(1). each of Q, Q¹, Q², Q³, Q⁴, Q⁵, and Q⁶ is:

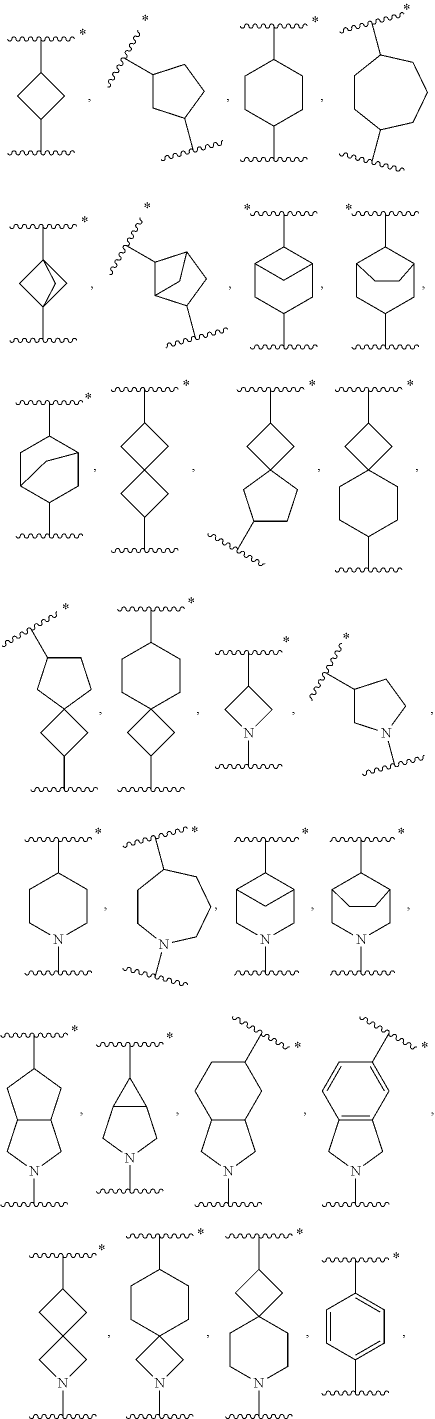

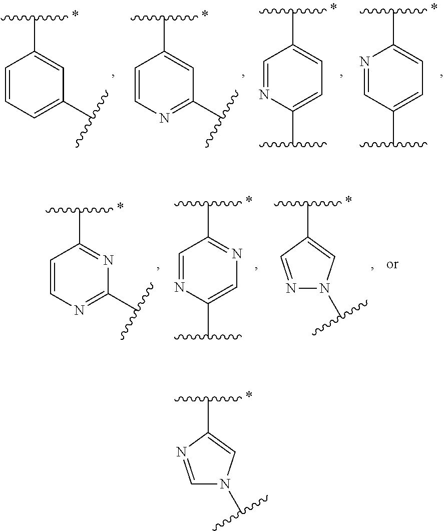

wherein each ring in (1) substituted with R^{v} and R^{w}; and
(2). heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
   heterocyclyl is:

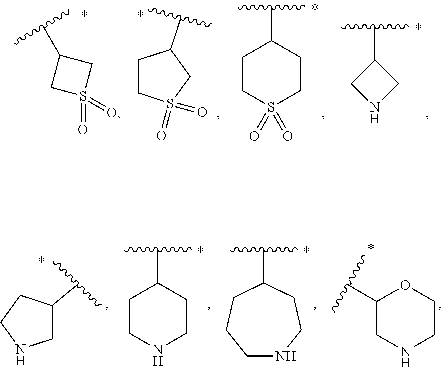

spiro heterocyclyl is:

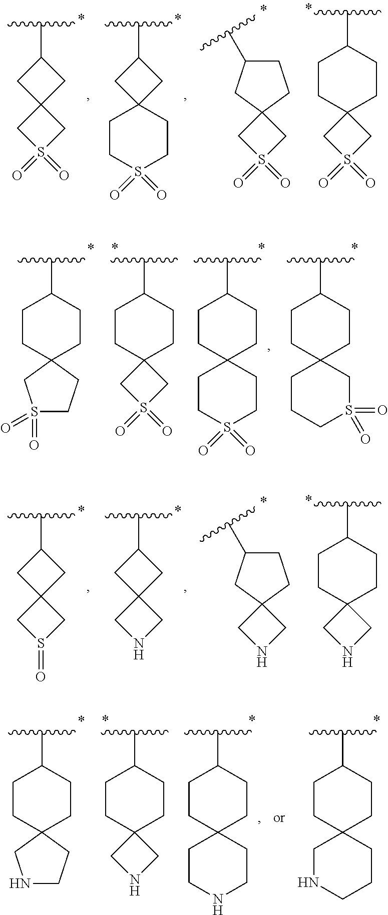

bridged heterocyclyl is:

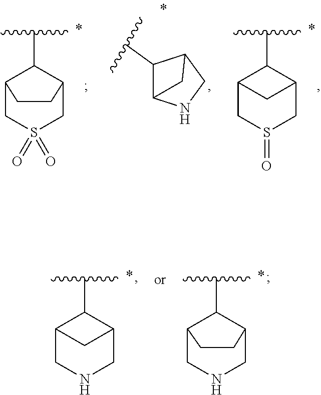

bicyclic heterocyclyl is:

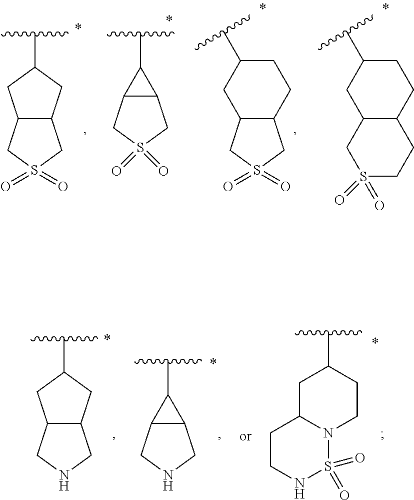

spiro sulfoximinyl is:

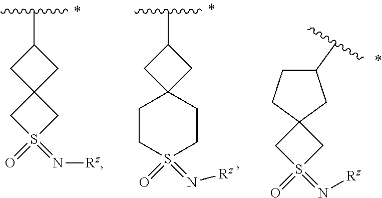

cyclylsulfoximinyl is:

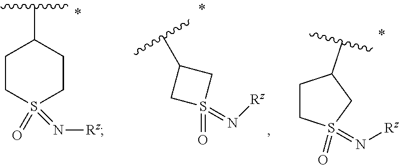

aryl is:

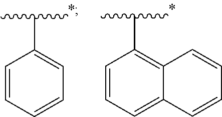

heteroaryl is:

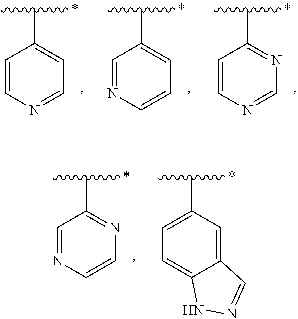 [SMILES: *c1ccncc1 ; *c1cccnc1 ; *c1ccncn1 ; *c1cnccn1 ; *c1ccc2[nH]ncc2c1]

fused heteroaryl is:

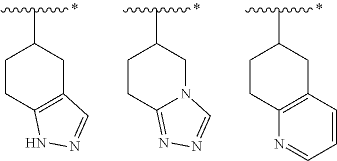

wherein each ring in (2) is substituted with R^{x}, R^{y}, and R^{y1}; and
   wherein

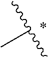

denotes the bond to NH of —NHR7 moiety of the compound of Formula (I) and

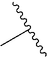

denotes bond to remainder of -Q-(alk¹)ₙ₁-SO₂R¹¹, -Q¹-(alk²)ₙ₂-SO(═NR¹²)R¹³, -Q²-(alk³)ₙ₃—SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, or -Q⁶-(alk⁶)ₙ₇-NR²¹SO₂R²².

61A. In embodiment 61A, the compound is as provided in any one of embodiments 1A to 60A, or a pharmaceutically acceptable salt thereof, wherein:
(1). each of Q, Q¹, Q², Q³, Q⁴, Q⁵, and Q⁶ is:

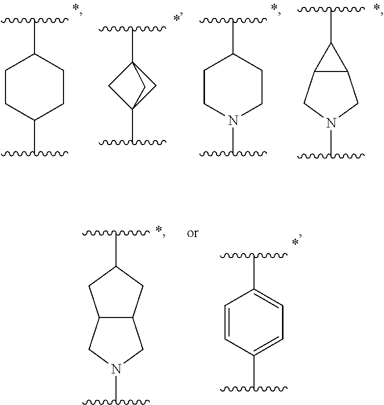

wherein each ring in (1) is substituted with R^{v} and R^{w} and

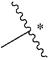

denotes the bond to NH of —NHR⁷ moiety of the compound of Formula (I) and

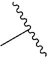

denotes bond to remainder of -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, or -Q⁶-(alk⁶)ₙ₇-NR²¹SO₂R²²; and
(2). heterocyclyl, bicyclic heterocyclyl, spiro heterocyclyl, spiro sulfoximinyl, aryl, and heteroaryl of R⁷ are where:
   heterocyclyl is:

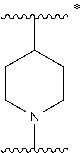 [SMILES: *C1CCN(*)CC1]

bicyclic heterocyclic is:

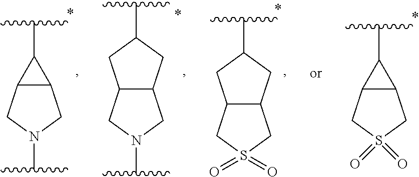 [SMILES: *C1C2CN(*)CC12 ; *C1CC2CN(*)CC2C1 ; *C1CC2CS(=O)(=O)CC2C1 ; *C1C2CS(=O)(=O)CC12]

spiro heterocyclyl is:

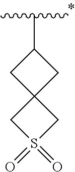 [SMILES: *C1CC2(C1)CS(=O)(=O)C2]

spirosulfoximinyl is:

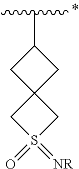

aryl is:

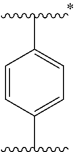 [SMILES: *c1ccc(*)cc1]

and heteroaryl is:

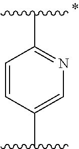 [SMILES: *c1ccc(*)nc1]

wherein each ring in (2) is substituted with R^{x}, R^{y}, and R^{y1} and

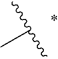

denotes the bond to NH of —NHR⁷ moiety of the compound of Formula (I) and

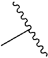

denotes attachment to one of R^{x}, R^{y}, and R^{y1}.

62A. In embodiment 62A, the compound is as provided in any one of embodiments 1A to 61A, or a pharmaceutically acceptable salt thereof, wherein each of Q, Q¹, Q², Q³, Q⁴, Q⁵, and Q⁶ is:

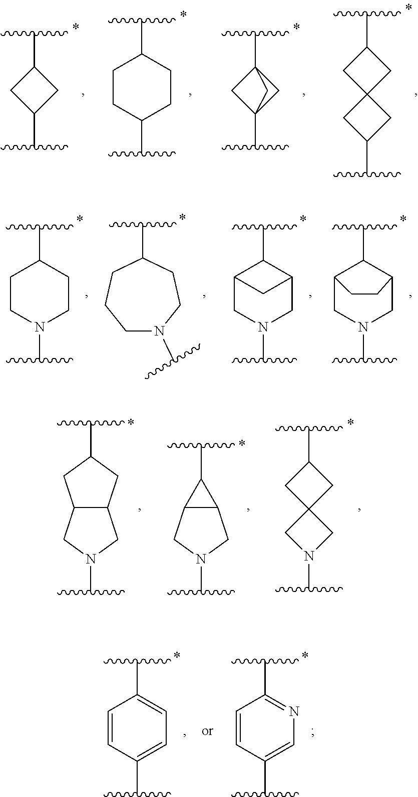

wherein each ring in (1) is substituted with R^{v} and R^{w}.

63A. In embodiment 63A, the compound is as provided in any one of embodiments 1A to 46A, 47A, 49B to 51A, and 58A to 62A, or a pharmaceutically acceptable salt thereof, wherein heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is:

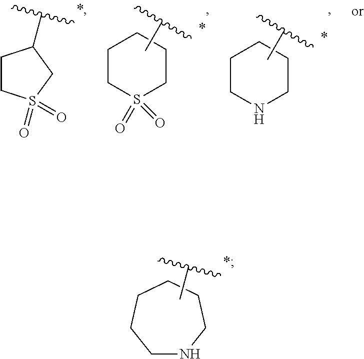

spiro heterocyclyl is:

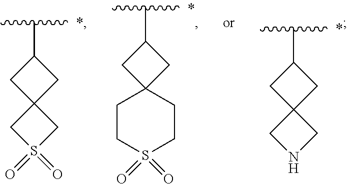 [SMILES: *C1CC2(C1)CS(=O)(=O)C2 ; *C1CC2(CCS(=O)(=O)CC2)C1 ; *C1CC2(CNC2)C1]

bridged heterocyclyl is:

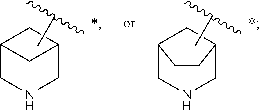

bicyclic heterocyclyl is:

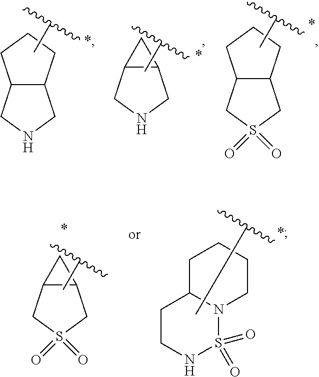

cyclylsulfoximinyl is:

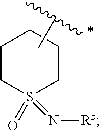

spiro sulfoximinyl is:

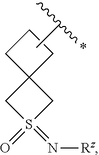

aryl is:

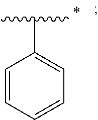 [SMILES: *c1ccccc1]

heteroaryl is:

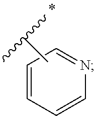

and fused heteroaryl is:

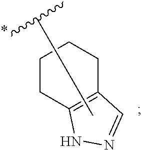

wherein each ring in (2) is substituted with R^{x}, R^{y}, and R^{y1}.

64A. In embodiment 64A, the compound is as provided in any one of embodiments 1A to 46A, 47A, 49B to SIA, and 58A to 63A, or a pharmaceutically acceptable salt thereof, wherein heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is:

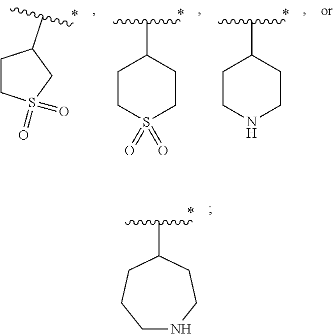 [SMILES: *C1CCS(=O)(=O)C1 ; *C1CCS(=O)(=O)CC1 ; *C1CCNCC1 ; *C1CCCNCC1]

spiro heterocyclyl is:

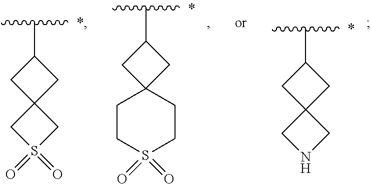 [SMILES: *C1CC2(C1)CS(=O)(=O)C2 ; *C1CC2(CCS(=O)(=O)CC2)C1 ; *C1CC2(CNC2)C1]

bridged heterocyclyl is:

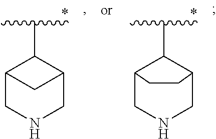 [SMILES: *C1C2CNCC1C2 ; *C1C2CCC1CNC2]

bicyclic heterocyclyl is:

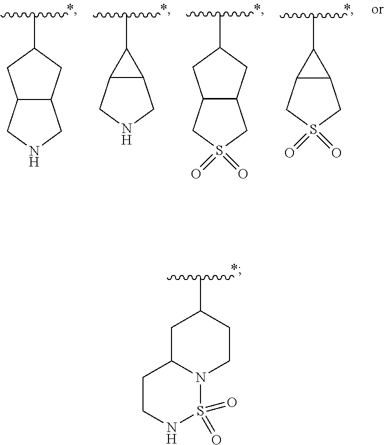

cyclylsulfoximinyl is:

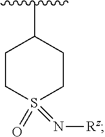

spiro sulfoximinyl is:

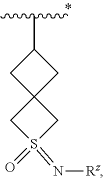

aryl is:

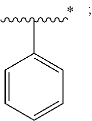 [SMILES: *c1ccccc1]

heteroaryl is:

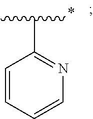 [SMILES: *c1ccccn1]

and fused heteroaryl is:

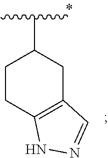 [SMILES: *C1CCc2[nH]ncc2C1]

wherein each ring is substituted with R^{x}, R^{y}, and R^{y1}.

65A. In embodiment 65A, the compound is as provided in any one of embodiments 1A to 46A, 47A, 49B to 51A, and 58A to 64A, or a pharmaceutically acceptable salt thereof, wherein heterocyclyl of R⁷ is: heterocyclyl is:

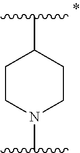 [SMILES: *C1CCN(*)CC1]

and is substituted with R^{x}, R^{y}, and R^{y1}.

66A. In embodiment 66A, the compound is as provided in any one of embodiments 1A to 46A, 47A, 49B to 51A, and 58A to 62A, or a pharmaceutically acceptable salt thereof, wherein spiro heterocyclyl, of R⁷ is

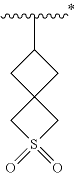 [SMILES: *C1CC2(C1)CS(=O)(=O)C2]

and is substituted with R^{x}, R^{y}, and R^{y1}.

67A. In embodiment 67A, the compound is as provided in any one of embodiments 1A to 46A, 47A, 49B to 51A, and 58A to 62A, or a pharmaceutically acceptable salt thereof, wherein bicyclic heterocyclyl, of R⁷ is: bicyclic heterocyclic is:

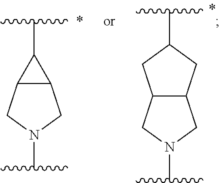 [SMILES: *C1C2CN(*)CC12 ; *C1CC2CN(*)CC2C1]

and each ring substituted with R^{x}, R^{y}, and R^{y1}.

68A. In embodiment 68A, the compound is as provided in any one of embodiments 1A to 46A, 47A, 49B to 51A, and 58A to 62A, or a pharmaceutically acceptable salt thereof, wherein aryl of R⁷ is:

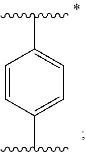 [SMILES: *c1ccc(*)cc1]

and substituted with R^{x}, R^{y}, and R^{y1}.

69A. In embodiment 69A, the compound is as provided in any one of embodiments 1A to 46A, 47A, 49B to 51, and 58A to 62A, or a pharmaceutically acceptable salt thereof, wherein bicyclic heterocyclyl of R⁷ is:

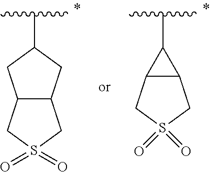 [SMILES: *C1CC2CS(=O)(=O)CC2C1 ; *C1C2CS(=O)(=O)CC12]

and each ring is substituted with R^{x}, R^{y}, and R^{y1}.

70A. In embodiment 70A, the compound is as provided in any one of embodiments 1A to 45A, and 52A to 69A, or a pharmaceutically acceptable salt thereof, wherein R¹², R¹⁹, and R²¹ are hydrogen.

71A. In embodiment 1A, the compound is as provided in any one of embodiments 1A to 45A, and 52A to 69A, or a pharmaceutically acceptable salt thereof, wherein R¹², R¹⁹, and R²¹ are methyl, ethyl, or propyl.

72A. In embodiment 71A, the compound is as provided in any one of embodiments 1A to 46A, 48A, 49A, and 52A to 71A, or a pharmaceutically acceptable salt thereof, wherein
R¹¹, R¹⁶, R²⁰, and R²² are independently selected from alkyl, fluoro, chloro, —CR²³═CR²⁴R²⁵ or heterocyclyl substituted with one to three substituents independently selected from hydrogen, alkyl, alkoxy, hydroxy, halo, haloalkyl, haloalkoxy, hydroxyalkyl, alkoxyalkyl, aminoalkyl, cyano, and heterocyclyl;
R¹³ is hydrogen, alkyl, haloalkyl, hydroxyalkyl, alkoxyalkyl, aminoalkyl, cycloalkyl; and
R¹⁴, R¹⁵, R¹⁷, and R¹⁸ are independently selected from hydrogen, alkyl, hydroxyalkyl, alkoxyalkyl, aminoalkyl, or heterocyclyl which is substituted with one to three substituents independently selected from hydrogen, alkyl, alkoxy, hydroxy, halo, haloalkyl, haloalkoxy, and cyano.

73A. In embodiment 73A, the compound is as provided in any one of embodiments 1A to 46A, 48A, 49A, and 52A to 73A, or a pharmaceutically acceptable salt thereof, wherein R¹¹ is methyl, ethyl, piperazinyl, 4-methylpiperazinyl, or 4-ethylpiperazin-1-yl; R¹⁴ is hydrogen, methyl, or ethyl, R¹⁵ is hydrogen, methyl, ethyl, aminoethyl, methylaminoethyl, dimethylaminoethyl, diethylaminoethyl, azetidin-3-yl, 1-methylazetidin-3-yl, 1-ethylazetidin-2-yl, 1-methylpyrrolidin-3-yl, 1-ethylpyrrolidin-3-yl, 1-methylpiperidinyl, or 1-ethylpiperidinyl.

74A. In embodiment 74A, the compound is as provided in any one of embodiments 1A to 46A, 47A, 49B to 51A, and 52A to 73A, or a pharmaceutically acceptable salt thereof, wherein R^{x}, R^{y}, and R^{y1} where R^{x} and R^{y} are independently selected from hydrogen, deuterium, alkyl, alkoxy, hydroxy, halo, haloalkyl, haloalkoxy, amino, alkylamino, dialkylamino, and cyano and R^{y1} is hydrogen, deuterium, alkyl, alkoxy, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, heterocyclylalkyloxy (wherein aryl, by itself or as part of aralkyl or aryloxy, heteroaryl, by itself or as part, of heteroaryloxy and heteroaralkyl, and heterocyclyl, by itself or as part of, heterocyclylalkyl, heterocyclyloxy, or heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocycyl)

75A. In embodiment 75A, the compound is as provided in any one of embodiments 1A to 46A, 47A, 49A to 51A, and 52A to 74A, or a pharmaceutically acceptable salt thereof, wherein R^{x} and R^{y} are independently selected from hydrogen, deuterium, alkyl, halo, haloalkyl, haloalkoxy, and cyano and RY′ is hydrogen, deuterium, alkyl, alkoxy, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, heterocyclylalkyloxy (wherein aryl, by itself or as part of aralkyl or aryloxy, heteroaryl, by itself or as part, of heteroaryloxy and heteroaralkyl, and heterocyclyl, by itself or as part of, heterocyclylalkyl, heterocyclyloxy, or heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl).

76A. In embodiment 76A, the compound is as provided in any one of embodiments 1A to 45A, or a pharmaceutically acceptable salt thereof, wherein R⁷ is 2,2-dioxido-2-thiaspiro[3.3]heptan-6-yl, 3,3-dioxido-3-thiabicyclo[3.1.0]hexan-6-yl, 4-(methylsulfonyl)-cyclohexyl, 2,2-dioxidohexahydro-1H-cyclopenta[c]thiophen-S-yl, 1-(methylsulfonyl)piperidin-4-yl, 1-(methylaminosulfonyl)piperidin-4-yl, 1-(aminosulfonyl)piperidin-4-yl, 4-(piperazin-1-yl)phenyl, 4-(piperazin-1-ylsulfonyl)piperidin-4-yl, 1-(N-(2-dimethylaminoethyl)-N-methylaminosulfonyl)piperidin-4-yl, 4-(4-methylpiperazin-1-ylsulfonyl)piperidin-4-yl, 5-(piperazin-1-yl)pyridin-2-yl, 4-(piperazin-1-yl)phenyl, 1-(N-(azetidin-3-yl)-N-methylaminosulfonyl)piperidin-4-yl, 1-(N-(1-methylazetidin-3-yl)-N-methylaminosulfonyl)-piperidin-4-yl, 2-(methylsulfonyl)-2-azaspiro[3.3]heptan-6-yl, 4-(4-(2-methoxyethyl)piperazin-1-yl)phenyl, 4-(2-(2-methoxyethoxy)ethyl)piperazin-1-yl)phenyl, 4-(4-(2-(2-methoxyethoxy)-ethyl)piperazin-1-yl)phenyl, 3-dimethylaminomethylphenyl, 4-methylsulfonylphenyl, 4-dimethylaminomethylphenyl, 4-(4-methylpiperazin-1-yl)phenyl, 4-(4-(oxetan-3-yl)piperazin-1-yl)phenyl, 1-(piperazin-1-ylsulfonyl)piperidin-4-yl, 3-((methylsulfonyl)methyl)bicyclo[1.1.1]-pentan-1-yl, or 2-imino-2-oxido-2-thiaspiro[3.3]heptan-6-yl.

77A. In embodiment 77A, the compound is as provided in any one of embodiments 1A to 45A, or a pharmaceutically acceptable salt thereof, wherein R⁷ is a ring of formula:

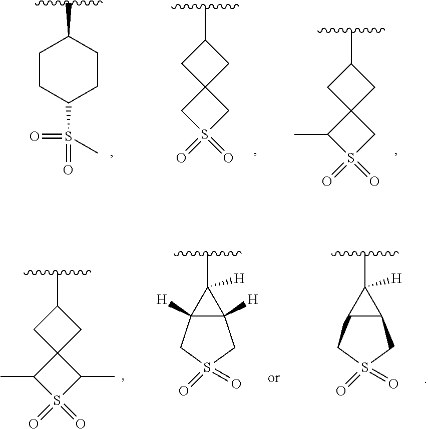

78A. In embodiment 78A, provided is a compound provided in Table 1.

79A. In embodiment 79A, provided is a pharmaceutical composition comprising a compound of as provided in any one of embodiments 1A to 78A, or a pharmaceutically acceptable salt thereof, and a pharmaceutically acceptable excipient.

80A. In embodiment 80A, provided is a method of inhibiting CDK2 which method comprises contacting CDK2 with a compound as provided in any one of embodiments 1A-78A, or a pharmaceutically acceptable salt thereof, or with a pharmaceutical composition as provided in embodiment 79A.

81A. In embodiment 81A, provided is a method of treating a disease mediated by CDK2 in a patient which method comprises administering to the patient in need thereof, a therapeutically effective amount of a compound as provided in any one of embodiments 1A-78A, or a pharmaceutically acceptable salt thereof, or with a pharmaceutical composition as provided in embodiment 79A.

82A. In embodiment 82A, provided is a method of treating cancer in a patient which method comprises administering to the patient in need thereof, a therapeutically effective amount of a compound as provided in any one of embodiments 1A-78A, or a pharmaceutically acceptable salt thereof, in a pharmaceutical composition as provided in embodiment 79A.

83A. In embodiment 83A, the method is as provided in embodiment 82A, wherein the compound is as provided in any one of embodiments 1A to 78A or a pharmaceutically acceptable salt thereof, or the pharmaceutical composition as provided in embodiment 79A, is administered in combination with at least one other anticancer agent.

84A. In embodiment 84A, the compound is as provided in embodiment 82A or 83A, wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.

### General Synthetic Scheme

Compounds of Formula (I) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of Formula (I) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) wherein

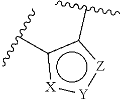

is

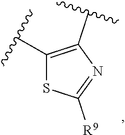

W is N and R¹, R², R³, R⁴, R⁵, R⁶, R⁷, R⁹ and R^{A}, are defined in the Summary (or any of the embodiments disclosed herein above) can be prepared as illustrated and described in Scheme 1 below.

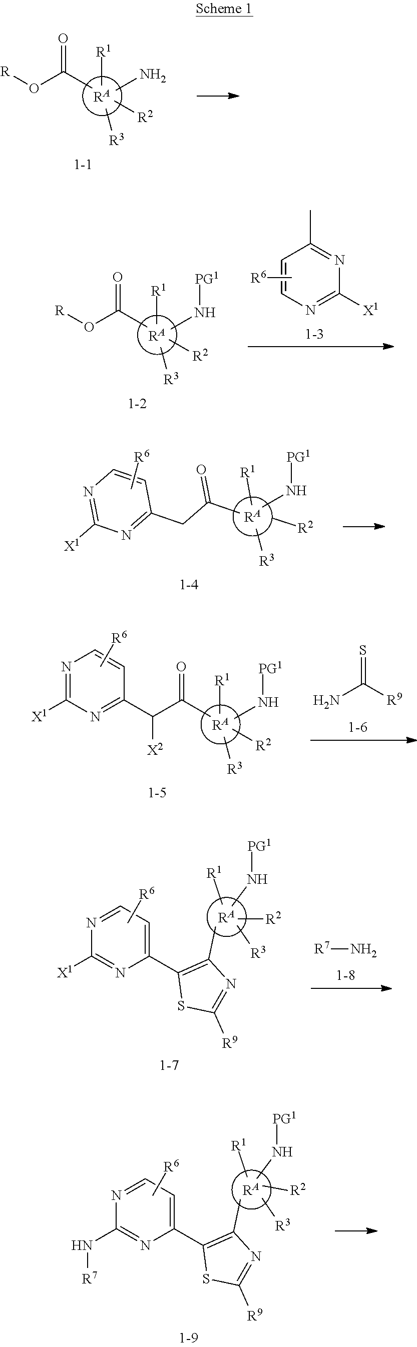

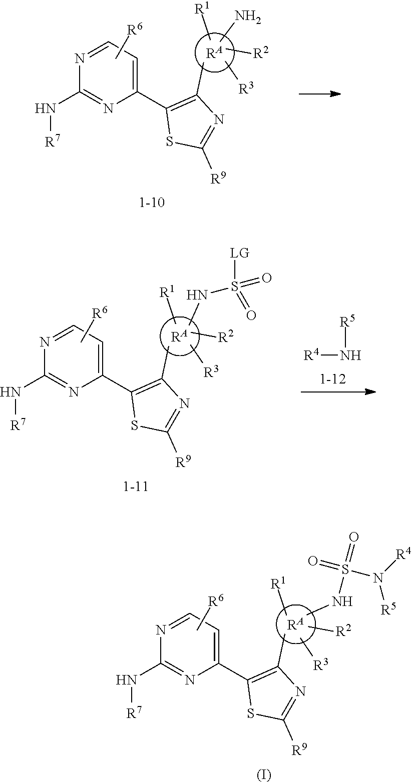

Protection of the amino group in compounds 1-1 where R is alkyl such as methyl and R¹, R², and R³ are as defined in the Summary (or any of the embodiments thereof above) or a precursor group thereof, with a suitable nitrogen protecting group (PG¹), such as acetyl, under conditions well known in the art provides a compound of formula 1-2. Treatment of compound 1-2 with a pyrimidine compound of formula 1-3 where X¹ is halogen, such as chlorine, bromine, in the presence of a base, such as LiHMDS, under conditions known in the art provides a ketone compound of formula 1-4. Compounds of formula 1-1 and 1-3 are commercially available or they can be prepared by methods well known in the art. For example, methyl 3-amino-2-fluorobenzoate, methyl 3-amino-4-fluorobenzoate, ethyl and 3-amino-2-fluorobenzoate are commercially available.

Halogenation of compound 1-4 with a suitable halogenation reagent, such as NBS, under conditions known in the art, followed by cyclization of the resulting compound 1-5 where X² is halogen, such as chlorine, bromine, with a thioamide of formula 1-6 where R⁹ is as defined in the Summary (or any of the embodiments thereof above) or a precursor group thereof (i.e., a group that can be converted to a R⁹ group is as defined in the Summary), provides a compound of formula 1-7. The reaction can be carried out in a suitable organic solvent, such as DMA, DMF, at elevated temperature. Compounds of formula 1-6 are commercially available or they can be prepared by methods known in the art. For example, 2,2-dimethylpropanethioamide, 3-(trifluoromethyl)-bicyclo[1.1.1]pentane-1-carbothioamide, (1R,5S)-3-methyl-3,8-diazabicyclo[3.2.1]octane-8-carbothioamide, thiourea are commercially available.

Treatment of a compound of formula 1-7 with an amine of formula 1-8 where R⁷ is as defined in the Summary (or any of the embodiments thereof above) or a precursor group thereof, provides a compound of 1-9. The reaction can be carried out in an organic solvent under basic conditions, such as in the presence of DIPEA, CsF, K₂CO₃, in DMSO or under Buchwald-type cross-coupling conditions. Typically, Buchwald-type cross-coupling conditions include a Pd catalyst, a ligand, and a base, for example a combination of Pd-RuPhos G2, and Cs₂CO₃. Removal of the amino protecting group PG¹ in 1-9 by treatment of compound of formula 1-9 with a suitable reagent, such as NaOH or HCl, under conditions well known in the art, provides an amine compound of formula 1-10. Amine of formula 1-8 are commercially available or they can be prepared by methods known in the art. For example, tert-butyl 4-(4-aminophenyl) piperazine-1-carboxylate, 1-(methylsulfonyl)piperidin-4-amine, (1r,4r)-4-(methylsulfonyl)cyclohexan-1-amine, and 6-amino-2-thiaspiro[3.3]heptane 2,2-dioxide are commercially available.

Sulfonamido compound of formula 1-11 where LG is a suitable leaving group such as chlorine, oxazolidin-2-one, can be prepared from amino compound of formula 1-10 under conditions known in the art. Treatment of compound of formula 1-11 with an amine 1-12 where R⁴ and R⁵ are as defined in the Summary (or any of the embodiments thereof above) or a precursor group thereof, in an organic solvent under basic conditions, such as in the presence of DIPEA, TEA, provides compound of Formula (I). Amine of formula 1-12 are commercially available or they can be prepared by methods known in the art. For example, indoline, isoindoline, dimethylamine, and piperidine are commercially available.

Alternatively compounds of Formula (I) wherein

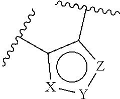

is

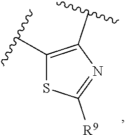

W is N and R¹, R², R³, R⁴, R⁵, R⁶, R⁷, R⁹ and R^{A}, are defined in the Summary (or any of the embodiments thereof above) can be prepared as illustrated and described in Scheme 2.

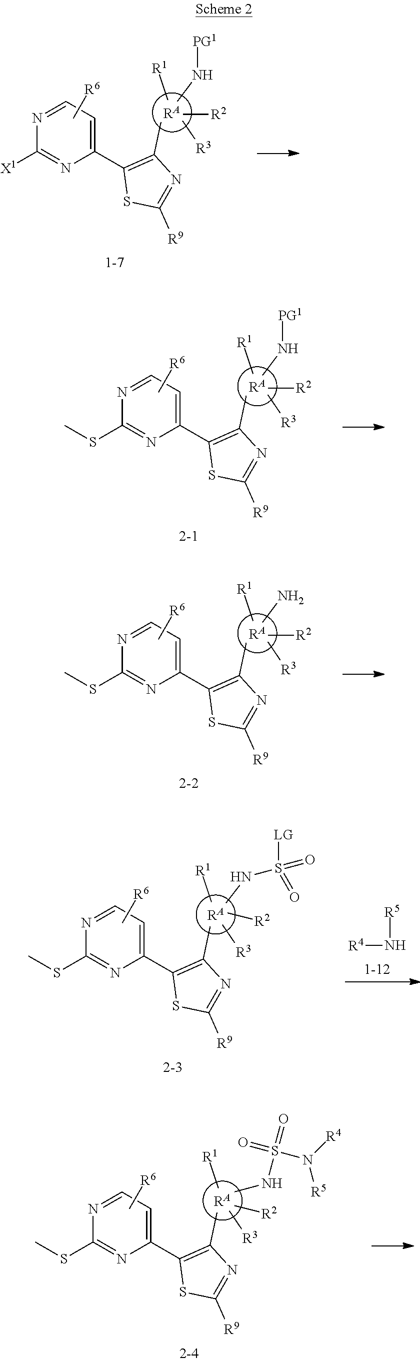

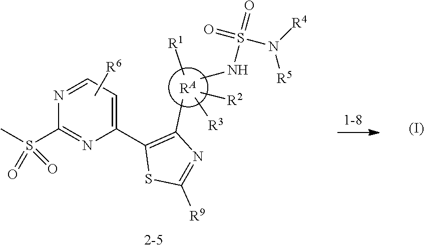

Treatment of compound 1-7 with a suitable thiol or thiol salt, such as sodium thiomethoxide, under reaction conditions known in the art, provides a methylthio compound of formula 2-1 which is converted to a compound of formula 2-4 by proceeding analogously as described in Scheme 1 above. Oxidation of compound 2-4 with a suitable oxidant such as Oxone, mCPBA, and the like, provides a methylsulfonyl compound of formula 2-5. Treatment of compound 2-5 with an amine of formula 1-8 where R⁷ is as defined in the Summary (or any of the embodiments thereof above) or a precursor group thereof, provides a compound of Formula (I). The reaction can be carried out in an organic solvent under basic conditions, such as in the presence of DIPEA, K₂CO₃, and the like, in a suitable solvent such as DMF, DMSO, and the like.

Alternatively, compounds of Formula (I) wherein

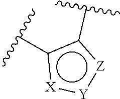

is

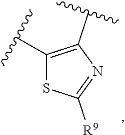

W is N and R¹, R², R³, R⁴, R⁵, R⁶, R⁷, R⁹ and R^{A}, are defined in the Summary (or any of the embodiments thereof above) can be prepared as illustrated and described in Scheme 3 below.

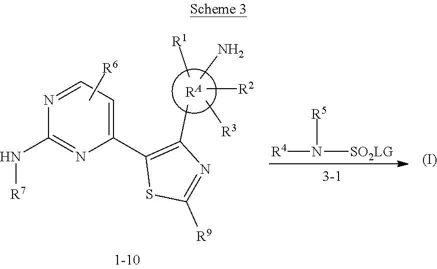

Treatment of amine 1-10 with an aminosulfonyl compound of formula 3-1 where R⁴ and R⁵ are as defined in the Summary (or any of the embodiments thereof above) or a precursor group thereof and LG is a leaving group, such as chlorine, bromine, and the like, in an organic solvent under basic conditions, such as in the presence of DIPEA, TEA, and the like, provides a compound of Formula (I). Compound of formula 3-1 is either commercially available or they can be prepared by methods known in the art and/or in Synthetic Examples below.

Alternatively, compounds of Formula (I) wherein

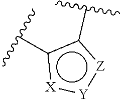

is

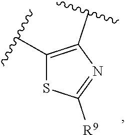

W is N and R¹, R², R³, R⁴, R⁵, R⁶, R⁷, R⁹ and R^{A}, are defined in the Summary (or any of the embodiments thereof above) can be prepared as illustrated and described in Scheme 4 below

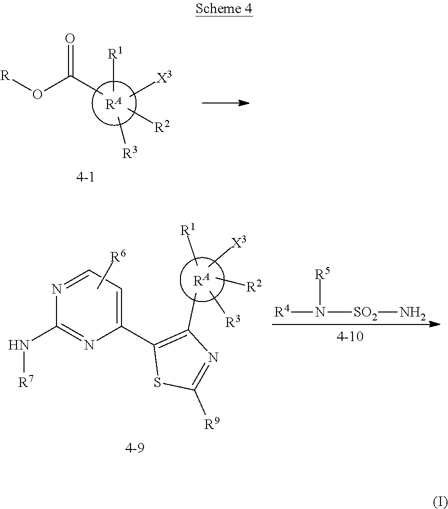

A compound of formula 4-1 where X³ is halogen, such as iodide, bromide, R is alkyl such as methyl, and R¹, R², and R³ are as defined in the Summary (or any of the embodiments thereof above) or a precursor group thereof, is converted to compound of formula 4-9 by proceeding analogously as described in Scheme 1 above. Treatment of compound 4-9 with amino sulfonamide compound of formula 4-10 where R⁴ and R⁵ are as defined in the Summary (or any of the embodiments thereof above), under coupling reaction conditions provides compound of Formula (I). The reaction can be carried out in an organic solvent in the presence of metal catalyst, such as in the presence of CuI, K₂CO₃, N¹,N²-dimethylcyclohexane-1,2-diamine in DMF or under Buchwald-type cross-coupling conditions. Typically, Buchwald-type cross-coupling conditions include a Pd catalyst, a ligand, and a base, for example a combination of Pd₂(dba)₃, XantPhos, Cs₂CO₃ in dioxane. Compounds of formula 4-10 are either commercially available or they can be prepared by methods known in the art and/or in Synthetic Examples below. For example, N,N-dimethylsulfamide, morpholine-4-sulfonamide, and indoline-1-sulfonamide are commercially available.

Compounds of Formula (I) wherein

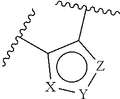

is

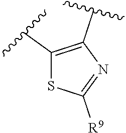

where R⁹ is heterocyclyl, bicyclic heterocyclyl, bridged heterocyclyl, and spiro heterocyclyl (each ring referred to in Scheme 5 as Het) attached to thiazolyl ring via ring nitrogen atom, W is N and R¹, R², R³, R⁴, R⁵, R⁶, R⁷, R⁹ and R^{A}, are defined in the Summary (or any of the embodiments thereof above) can be prepared as illustrated and described in Scheme 5 below.

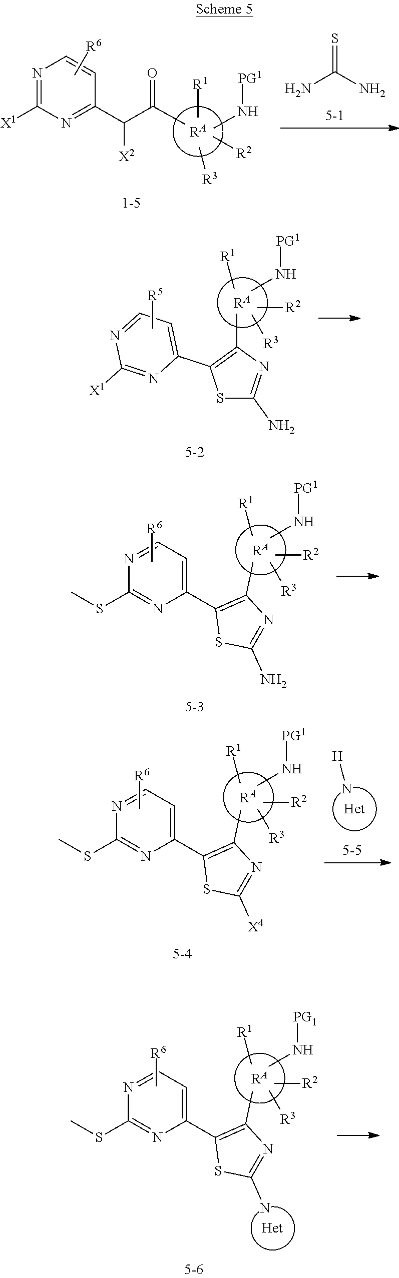

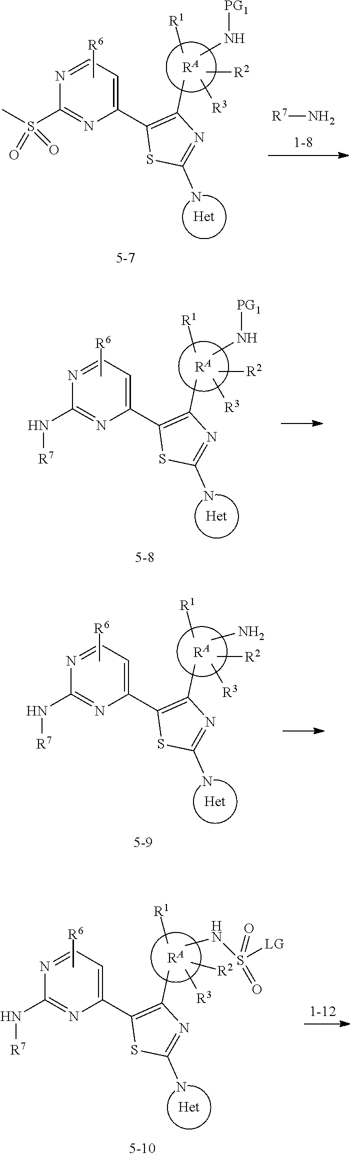

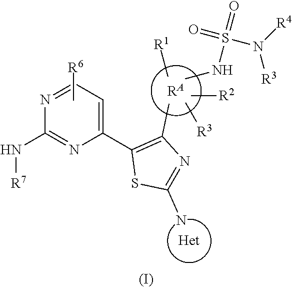

Cyclization of a compound of formula 1-5 as defined in Scheme 1 above, with thiourea under conditions analogous as described in Scheme 1 above, provides a compound of formula 5-2 which is converted to a compound of formula 5-3 under conditions analogous as described in Scheme 2 above. Compound 5-3 is then converted to a compound of formula 5-4 where X⁴ is a halogen such as chlorine or bromine, under reaction conditions known in the art. For example, compound 5-4 where X⁴ is bromo can be prepared by treatment of compound 5-3 with tert-butyl nitrite and CuBr₂. Treatment of a compound of formula 5-4 with an amine of formula 5-5 where Het is heterocyclyl, bicyclic heterocyclyl, bridged heterocyclyl, and spiro heterocyclyl containing at least one ring nitrogen atom provides a compound of formula 5-6. The reaction can be carried out in an organic solvent under basic conditions, such as in the presence of DIPEA, K₂CO₃, and the like, in DMSO at elevated temperature. Thiol ether compound of formula 5-6 is converted to compound of formula 5-8 by proceeding analogously as described in Scheme 2, and compound 5-8 is then compound of Formula (I) as described in Scheme 1 above.

Compounds of Formula (I) wherein

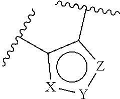

is

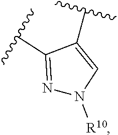

W is N and R¹, R², R³, R⁴, R⁵, R⁶, R⁷, R¹⁰ and R^{A}, are defined in the Summary (or any of the embodiments thereof above) can be prepared as illustrated and described in Scheme 6 below.

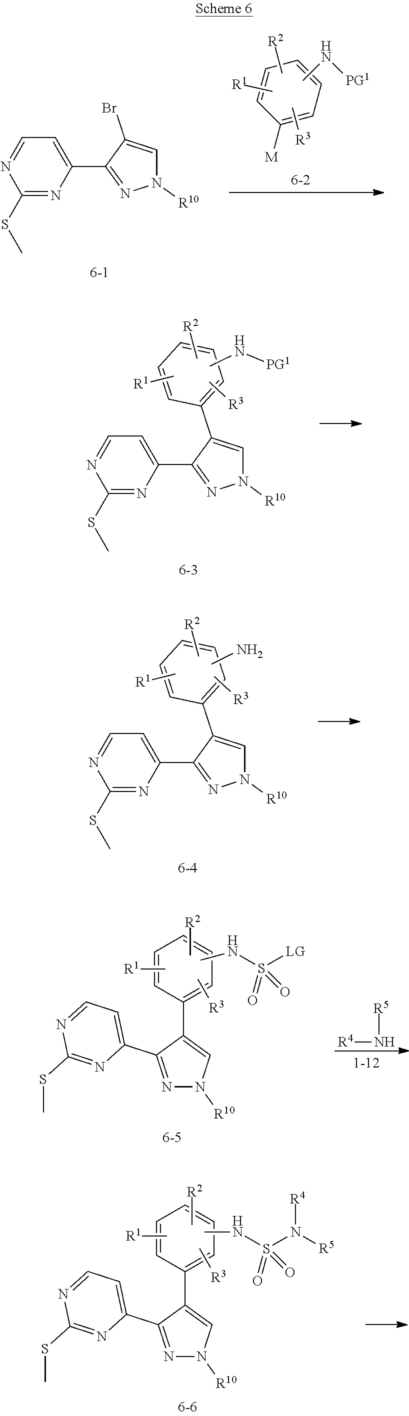

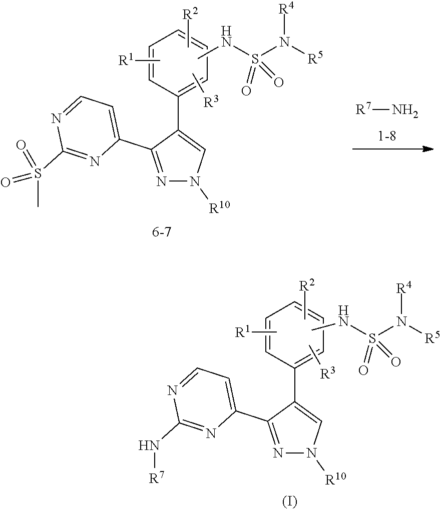

Treatment of the bromide compound 6-1 where R¹⁰ is defined as in Summary (or any of the embodiments thereof above), with compound of formula 6-2 where PG¹ is a suitable amine protecting group (such as acetyl) and M is a metal (such as boron or zinc) under cross coupling reaction conditions known in the art, e.g., Suzuki reaction (when M is boron) in the presence of a palladium catalyst, a base, and suitable solvents, provides compound of formula 6-3. Compounds of formula 6-1 can be prepared by methods known in the art e.g., PCT application publication No. WO2012125981. Compounds of formula 6-2 are commercially available (e.g., (N-[2-fluoro-3-(tetramethyl-1,3,2-dioxaborolan-2-yl)phenyl]acetamide) or they can be prepared by methods known in the art. Thiol ether compound 6-3 can be converted to a compound of Formula (I) under conditions analogous to Scheme 2.

Compounds of Formula (I) wherein

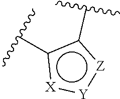

is

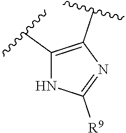

where W is N, R^{A} is phenyl, and R¹, R², R³, R⁴, R⁵, R⁶, R⁷, and R¹⁰ are defined in the Summary (or any of the embodiments thereof above) can be prepared as illustrated and described in Scheme 7 below.

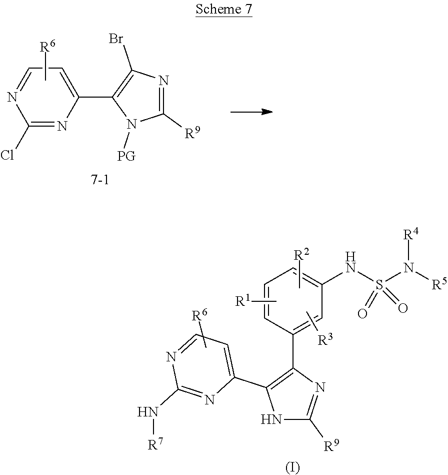

Imidazole of formula 7-1 can be converted to a compound of Formula (I) by proceeding as described in Scheme 6 above. Compounds of formula 7-1 can be prepared by methods described in US patent application publication No. US20110052578A1,

Compounds of Formula (I) wherein

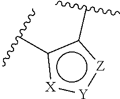

is

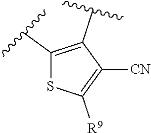

where R⁹ is heterocyclyl, bicyclic heterocyclyl, bridged heterocyclyl, or spiro heterocyclyl (referred to herein as Het) attached to thienyl ring via ring nitrogen atom, W is N, R^{A} is phenyl, and R¹, R², R³, R⁴, R⁵, R⁶, and R⁷ are defined in the Summary (or any of the embodiments thereof above) can be prepared as illustrated and described in Scheme 8 below.

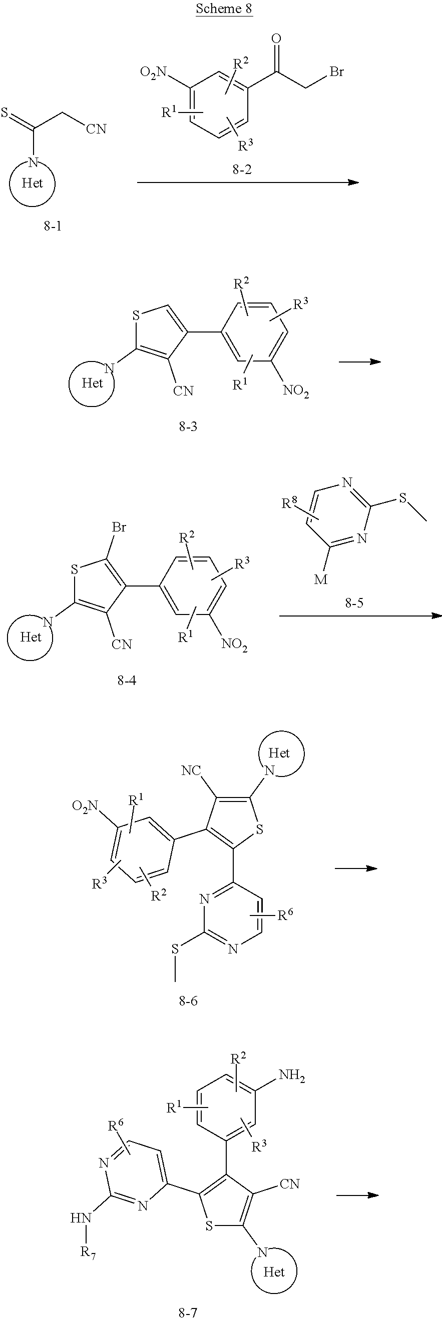

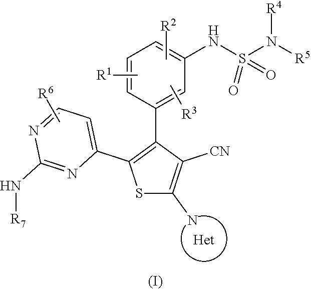

Thiophene of formula 8-3 can be synthesized by reacting a compound of formula 8-1 where Het is heterocyclyl, bicyclic heterocyclyl, bridged heterocyclyl, or spiro heterocyclyl (or any embodiments thereof hereinabove) with a compound of formula 8-2 where R¹ and R² are as defined in the Summary (or an embodiment thereof above) by proceeding analogously as described in Korean patent application publication No. KR20180024945A. Treatment of compound 8-3 with a brominating agent, such as NBS, provides a compound of formula 8-4. A cross coupling reaction between 8-4 and a pyrimidine of formula 8-5 where M is a metal, such as trialkyltin or boronic ester, gives a compound of formula 8-6. The cross-coupling reactions typically include a Pd catalyst, a ligand, and a base, for example a combination of Pd₂(dba)₃, PPh₃, CsF in dioxane when M is tri-n-butyltin. Reduction of nitro group in compound 8-6, e.g., palladium catalyst under H₂ atmosphere, provides an amine compound of formula 8-7 which can be converted to compound of Formula (I) by proceeding as described in Scheme 6 above Het is heterocyclyl, bicyclic heterocyclyl, bridged heterocyclyl, or spiro heterocyclyl attached to thiazolyl ring via ring nitrogen atom.

Compounds of Formula (I) can be converted to other compounds of Formula (I) by methods well known in the art. For example, compound of Formula (I) where R⁹ is, for example, piperazin-1-yl substituted at the 2^{nd} nitrogen ring atom of the piperazin-1-yl ring with R$ where R$ is H can be converted to corresponding compounds of Formula (I) where R^{b} is alkyl or cycloalkyl by reacting it with alkyl or cycloalkyl ketones under reductive amination conditions to give corresponding compounds of Formula (I) where R^{b} is alkyl or cycloalkyl.

Compounds of Formula (I) where R⁷ is piperidin-4-yl can be reacted with a carboxylic acid of formula R¹⁶COOH under amide coupling reaction conditions or a sulfonylating reagent of formula R¹¹SO₂X where X is halide and R¹¹ and R¹⁶ are as defined in the Summary to give corresponding compounds of Formula (I) where R⁷ is 1-(R¹⁶CO-)piperidin-4yl and 1-(R¹¹SO₂-)piperidin-4yl, respectively.

### Utility

Increasing evidence suggests that over-activated CDK2 leads to abnormal cell cycle regulation and proliferation in cancer cells. The kinase activity of CDK2/Cyclin B or CDK2/Cyclin A complex is elevated via several mechanisms in human cancers. Cyclin E has been found to be frequently amplified, for example, in uterine cancer, ovarian cancer, stomach cancer, and breast cancer. In some cancer types, loss-of-function mutations in FBXW7 or overexpression of USP28, which control the turnover of cyclin E, leads to cyclin E overexpression and CDK2 activation. Alternatively, certain cancer cells express a hyperactive, truncated form of cyclin E or cyclin A. In addition, cyclin A amplification and overexpression have also been reported in various cancers such as hepatocellular carcinomas, colorectal and breast cancers. In some tumors, catalytic activity of CDK2 is increased following loss of the expression or alteration of the location of the endogenous CDK2 inhibitor p27 or p21, or overexpression of SKP2, a negative regulator of p27. In addition, CDC25A and CDC25B, protein phosphatases responsible for the dephosphorylations that activate the CDK2, are overexpressed in various tumors. These various mechanisms of CDK2 activation have been validated using cancer cells or mouse cancer models. Furthermore, CDK2/cyclin E phosphorylates oncogenic Myc to oppose ras-induced senescence, highlighting the importance of CDK2 in myc/ras-induced tumorigenesis. Inactivation of CDK2 has been shown to be synthetically lethal to myc over-expressing cancer cells. In aneuploid cancer cells, for example KRAS-mutant lung cancer, CDK2 inhibition resulted in anaphase catastrophe and apoptosis. Moreover, inhibiting CDK2 effectively induced granulocytic differentiation in AML cell lines and arrested tumor growth in AML mice models.

CDK2 activation as a result of cyclin E amplification or overexpression has also been identified as a key primary or acquired resistance pathway to tumors treated by CDK4/6 inhibitors or trastuzumab. Accordingly, compounds of Formula (I) can be used in combination with CDK4/6 inhibitors for the treatment of cancers that become refractory to CDK4/6 inhibitors.

Thus, compounds of Formula (I) or a pharmaceutically acceptable salt thereof, may be useful for treating tumors characterized by one or more of: overexpression of CDK2, hyperphosphorylation of CDK2 (Thr160), amplification/overexpression of cyclin E or cyclin A, RB-deficiency, loss-of-function of mutation in FBXW7 or overexpression of USP28, expression of truncated cyclin E or cyclin A, dysregulation of p21 or p27 or overexpression of SKP2, amplification/overexpression of CDC25A or/and CDC25B, depletion of AMBRA1, hyperactive MYC/RAS, Aneuploid cancers, CDK4 and/or CDK6 inhibitor refractory cancers.

In some embodiments, the cancer is ovarian cancer, endometrial cancer, breast cancer (e.g., triple negative breast cancer), lung cancer (e.g., adenocarcinoma, small cell lung cancer and non-small cell lung carcinomas, parvicellular and non-parvicellular carcinoma, bronchial carcinoma, bronchial adenoma, and/or pleuropulmonary blastoma), skin cancer (e.g. melanoma, squamous cell carcinoma, Kaposi sarcoma, and/or Merkel cell skin cancer), bladder cancer, cervical cancer, colorectal cancer, cancer of the small intestine, colon cancer, rectal cancer, cancer of the anus, gastric cancer, head and neck cancer (e.g., cancers of the larynx, hypopharynx, nasopharynx, oropharynx, lips, and/or mouth), liver cancer (e.g., hepatocellular carcinoma, and/or cholangiocellular carcinoma), prostate cancer, testicular cancer, uterine cancer, esophageal cancer, gall bladder cancer, pancreatic cancer (e.g. exocrine pancreatic carcinoma), stomach cancer, thyroid cancer, brain cancer, fallopian tube cancer, peritoneal cancer, AML, and parathyroid cancer. In some embodiments, the cancer is ovarian cancer. In some such embodiments, the ovarian cancer is characterized by amplification or overexpression of CCNE1 and/or CCNE2.

In other embodiments, the cancer is breast cancer, including, e.g., ER-positive/HR-positive breast cancer, HER2-negative breast cancer; ER-positive/HR-positive breast cancer, HER2-positive breast cancer; triple negative breast cancer (TNBC); or inflammatory breast cancer. In some embodiments, the breast cancer is endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification or overexpression of CCNE1, CCNE2, and/or CCNA2.

Besides cancer, CDK2 upregulation is also implicated in autoimmune diseases (e.g., rheumatoid arthritis (RA), systemic lupus erythematosus (SLE), primary Sjogren's syndrome (pSS), multiple sclerosis (MS), Crohn's disease (CD), gout, uveitis, and pemphigus vulgaris) and sepsis.

### Testing

The CDK2 inhibitory activity of the compounds of the present disclosure can be tested using the in vitro assay described in Biological Example 1 below.

### Pharmaceutical Compositions

In general, the compounds of Formula (I) (unless stated otherwise, reference to compound/compounds of Formula (I) herein includes any embodiments thereof described herein or a pharmaceutically acceptable salt thereof) will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound Formula (I), i.e., the active ingredient, will depend upon numerous factors such as the severity of the disease to be treated, the age and relative health of the patient, the potency of the compound being utilized, the route and form of administration, and other factors.

In general, compounds of Formula (I) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formula (I) in combination with at least one pharmaceutically acceptable excipient. Acceptable excipients are generally non-toxic, aid administration, and do not adversely affect the therapeutic benefit of the compound of Formula (I). Such excipient may be any solid, liquid, semi-solid or, in the case of an aerosol composition, gaseous excipient that is generally available to one of skill in the art.

Solid pharmaceutical excipients include starch, cellulose, tale, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formula (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formula (I) may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formula (I) may also be formulated in rectal compositions such as suppositories or retention enemas, e.g., containing conventional suppository bases such as cocoa butter, polyethylene glycol, or other glycerides.

Certain compounds of Formula (I) may be administered topically, that is by non-systemic administration. This includes the application of a compound of Formula (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds of Formula (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formula (I) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formula (I) in a formulation can vary within the full range employed by those skilled in the art. Typically, the formulation will contain, on a weight percent (wt. %) basis, from about 0.01-99.99 wt. % of a compound of Formula (I) based on the total formulation, with the balance being one or more suitable pharmaceutical excipients. For example, the compound is present at a level of about 1-80 wt. %.

### Combinations and Combination Therapies

The compounds of Formula (I) may be used in combination with one or more other drugs in the treatment of diseases or conditions for which compounds of Formula (I) or the other drugs may have utility. Such other drug(s) may be administered, by a route and in an amount commonly used therefore, contemporaneously or sequentially with a compound of Formula (I). When a compound of Formula (I) is used contemporaneously with one or more other drugs, a pharmaceutical composition in unit dosage form containing such other drugs and the compound of Formula (I) is preferred. However, the combination therapy may also include therapies in which the compound of Formula (I) and one or more other drugs are administered on different overlapping schedules. It is also contemplated that when used in combination with one or more other active ingredients, the compounds of Formula (I) and the other active ingredients may be used in lower doses than when each is used singly.

Accordingly, the pharmaceutical compositions of the present disclosure also include those that contain one or more other drugs, in addition to a compound of Formula (I).

The above combinations include combinations of a compound of Formula (I) not only with one other drug, but also with two or more other active drugs. Likewise, a compound of Formula (I) may be used in combination with other drugs that are used in the prevention, treatment, control, amelioration, or reduction of risk of the diseases or conditions for which a compound of Formula (I) is useful. Such other drugs may be administered, by a route and in an amount commonly used therefore, contemporaneously or sequentially with a compound of Formula (I). When a compound of Formula (I) is used contemporaneously with one or more other drugs, a pharmaceutical composition containing such other drugs in addition to the compound of Formula (I) can be used. Accordingly, the pharmaceutical compositions of the present disclosure also include those that also contain one or more other active ingredients, in addition to a compound of Formula (I). The weight ratio of the compound of this disclosure to the second active ingredient may be varied and will depend upon the effective dose of each ingredient. Generally, an effective dose of each will be used.

Where the subject in need is suffering from or at risk of suffering from cancer, the subject can be treated with a compound of Formula (D) in any combination with one or more other anti-cancer agents including but not limited to: MAP kinase pathway (RAS/RAF/MEK/ERK) inhibitors including but not limited to: Vemurafanib (PLX4032), Dabrafenib, Encorafenib (LGX818), TQ-B3233, XL-518 (Cas No. 1029872-29-4, available from ACC Corp); trametinib, selumetinib (AZD6244), TQ-B3234, PD184352, PD325901, TAK-733, pimasertinib, binimetinib, refametinib, cobimetinib (GDC-0973), AZD8330, BVD-523, LTT462, Ulixertinib, AMG$10 (sotorasib), ARS853, adagrasib, opnurasib, divarasib, LY3537982 (2-amino-4-[(4aS)-8-chloro-10-fluoro-2,3,4,4a,5,6-hexahydro-12-oxo-3-(1-oxo-2-propen-1-yl)-1H,12H-pyrazino[2,1-d][1,5]benzoxazocin-9-yl]-7-fluorobenzo[b]thiophene-3-carbonitrile), MRTX1133 (4-(4-((1R,5S)-3,8-diazabicyclo[3.2.1]octan-3-yl)-8-fluoro-2-(((2R,7aS)-2-fluorotetrahydro-1H-pyrrolizin-7a (5H)-yl) methoxy)pyrido[4,3-d]pyrimidin-7-yl)-5-ethynyl-6-fluoronaphthalen-2-ol), RMC-6291, RMC-9805, RMC0708, RMC-8839 and any RAS inhibitors disclosed in PCT Publication Nos. WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, and WO2018119183;
CSF1R inhibitors (PLX3397, LY3022855, etc.) and CSFIR antibodies (IMC-054, RG7155);
TGF beta receptor kinase inhibitor such as LY2157299;
BTK inhibitor such as ibrutinib; BCR-ABL inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-(cyclobutylarmno)-5-(trifluoromethyl)-2-pyrimidinyl]amino]-1,2,3,4-tetrahydronaphthalen-1,4-imin-9-yl]-2-oxoethyl]-acetamide (PF-03814735, CAS 942487-16-3);
ALK inhibitors: PF-2341066 (XALKOPJ®; crizotinib); 5-chloro-N4-(2-(isopropylsulfonyl)phenyl)-N2-(2-methoxy-4-(4-(4-methylpiperazin-1-yl) piperidin-1-yl)phenyl)pyrimidine-2,4-diamine; GSK1838705 A; CH5424802; Ceritinib (ZYKADIA); TQ-B3139, TQ-B3101 PI3K inhibitors: 4-[2-(1H-indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730), 2-methyl-2-[4-[3-methyl-2-oxo-8-(quinolin-3-yl)-2,3-dihydroimidazo[4,5-c]quinolin-|-yl]phenyl]propionitrile (also known as BEZ 235 or NVP-BEZ 235, and described in PCT Publication No. WO 06/122806);
Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01/002369), Brivanib Alaninate ((S)-((R)-1-(4-(4-fluoro-2-methyl-1H-indol-5-yloxy)-5-methylpyrrolo[2,1-f][1,2,4]triazin-6-yloxy)propan-2-yl)-2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide, and described in PCT Publication No. WO 02/066470), pasireotide (also known as SOM230, and described in PCT Publication No. WO 02/010192), sorafenib (sold under the tradename Nexavart); AL-2846 MET inhibitor such as foretinib, carbozantinib, or crizotinib;
FLT3 inhibitors-sunitinib malate (sold under the tradename Sutent® by Pfizer); PKC412 (midostaurin); tanutinib, sorafenib, lestaurtinib, KW-2449, quizartinib (AC220) and crenolanib;
Epidermal growth factor receptor (EGFR) inhibitors: Gefitnib (sold under the tradename Iressa®), N-[4-[(3-chloro-4-fluorophenyl)amino]-7-[[(3S)-tetrahydro-3-furanyl]oxy]-6-quinazolinyl]-4 (dimethylamino)-2-butenamide, sold under the tradename Tovok® by Boehringer Ingelheim), cetuximab (sold under the tradename Erbitux® by Bristol-Myers Squibb), panitumumab (sold under the tradename Vectibix® by Amgen);
HER2 receptor inhibitors: Trastuzumab (sold under the trademark Herceptin® by Genentech/Roche), neratinib (also known as HKI-272, (2E)-N-[4-[[3-chloro-4-[(pyridin-2-yl) methoxy]phenyl]amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by GlaxoSmithKline); Trastuzumab emtansine (in the United States, ado-trastuzumab emtansine, trade name Kadcyla)—an antibody-drug conjugate consisting of the monoclonal antibody trastuzumab (Herceptin) linked to the cytotoxic agent mertansine (DM1); HER dimerization inhibitors: Pertuzumab (sold under the trademark Omnitarg®, by Genentech); CD20 antibodies: Rituximab (sold under the trademarks Riuxan® and Mab Thera® by Genentech/Roche), tositumomab (sold under the trademarks Bexxar® by GlaxoSmithKline), ofatumumab (sold under the trademark Arzerra® by GlaxoSmithKline);
Tyrosine kinase inhibitors: Erlotinib hydrochloride (sold under the trademark Tarceva® by Genentech/Roche), Linifanib (N-[4-(3-amino-1H-indazol-4-yl)phenyl]-N′-(2-fluoro-5-methylphenyl) urea, also known as ABT-869, available from Genentech), sunitinib malate (sold under the tradename Sutent® by Pfizer), bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996), dasatinib (sold under the tradename Sprycel® by Bristol-Myers Squibb), armala (also known as pazopanib, sold under the tradename Votrient® by GlaxoSmithKline), imatinib and imatinib mesylate (sold under the tradenames Gilvec® and Gleevec® by Novartis);
DNA Synthesis inhibitors: Capecitabine (sold under the trademark Xeloda® by Roche), gemcitabine hydrochloride (sold under the trademark Gemzar® by Eli Lilly and Company), nelarabine ((2R3S,4R,5R)-2-(2-amino-6-methoxy-purin-9-yl)-5-(hydroxymethyl)oxolane-3,4-diol, sold under the tradenames Arranon® and Atriance® by GlaxoSmithKline);
Antineoplastic agents: oxaliplatin (sold under the tradename Eloxatin® ay Sanofi-Aventis and described in U.S. Pat. No. 4,169,846);
Human Granulocyte colony-stimulating factor (G-CSF) modulators: Filgrastim (sold under the tradename Neupogen® by Amgen).
Immunomodulators: Afutuzumab (available from Roche®), pegfilgrastim (sold under the tradename Neulasta® by Amgen), lenalidomide (also known as CC-5013, sold under the tradename Revlimid®), thalidomide (sold under the tradename Thalomid®);
CD40 inhibitors: Dacetuzumab (also known as SGN-40 or huS2C6, available from Seattle Genetics, Inc); Pro-apoptotic receptor agonists (PARAs): Dulanermin (also known as AMG-951, available from Amgen/Genentech);
Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958); Phospholipase A2 inhibitors: Anagrelide (sold under the tradename Agrylin®);
BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386);
MCI-1 inhibitors: MIK665, S64315, AMG 397, and AZD5991;
Aromatase inhibitors: Exemestane (sold under the trademark Aromasin® by Pfizer), letrozole (sold under the tradename Femara® by Novartis), anastrozole (sold under the tradename Arimidex®);
Topoisomerase I inhibitors: Irinotecan (sold under the trademark Camptosar® by Pfizer), topotecan hydrochloride (sold under the tradename Hycamtin® by GlaxoSmithKline);
Topoisomerase II inhibitors: etoposide (also known as VP-16 and Etoposide phosphate, sold under the tradenames Toposar®, VePesid® and Etopophos®), teniposide (also known as VM-26, sold under the tradename Vumon®);
mTOR inhibitors: Temsirolimus (sold under the tradename Torisek® by Pfizer), ridaforolimus (formally known as deferolimus, (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0 4′9]hexatriaconta-16,24,26,28-tetraen-12-yl]propyl]-2-methoxycyclohexyl dimethylphosphinate, also known as AP23573 and MK8669, and described in PCT Publication No. WO 03/064383), everolimus (sold under the tradename Afinitor® by Novartis);
Proteasome inhibitor such as carfilzomib, MLN9708, delanzomib, or bortezomib;
BET inhibitors such as INCB054329, OTX015, and CPI-0610;
LSD1 inhibitors such as GSK2979552, and INCB059872;
HIF-2α inhibitors such as PT2977 and PT2385;
Osteoclastic bone resorption inhibitors: I-hydroxy-2-imidazol-1-yl-phosphonoethyl)phosphonic acid monohydrate (sold under the tradename Zometa® by Novartis); CD33 Antibody Drug Conjugates: Gemtuzumab ozogamicin (sold under the tradename Mylotarg® by Pfizer/Wyeth);
CD22 Antibody Drug Conjugates: Inotuzumab ozogamicin (also referred to as CMC-544 and WAY-207294, available from Hangzhou Sage Chemical Co., Ltd.);
CD20 Antibody Drug Conjugates: Ibritumomab tiuxetan (sold under the tradename Zevalin®);
Somatostain analogs: octreotide (also known as octreotide acetate, sold under the tradenames Sandostatin® and Sandostatin LAR®);
Synthetic Interleukin-11 (IL-11): oprelvekin (sold under the tradename Neumega® by Pfizer/Wyeth);
Synthetic erythropoietin: Darbepoetin alfa (sold under the tradename Aranesp® by Amgen);
Receptor Activator for Nuclear Factor κ B (RANK) inhibitors: Denosumab (sold under the tradename Prolia® by Amgen);
Thrombopoietin mimetic peptibodies: Romiplostim (sold under the tradename Nplate® by Amgen;
Cell growth stimulators: Palifermin (sold under the tradename Kepivance®) by Amgen); Anti-Insulin-like Growth Factor-1 receptor (IGF-1R) antibodies: Figitumumab (also known as CP-751,871, available from ACC Corp), robatumumab (CAS No. 934235-44-6);
Anti-CSI antibodies: Elotuzumab (HuLuc63, CAS No. 915296-00-3);
CD52 antibodies: Alemtuzumab (sold under the tradename Campath®);
Histone deacetylase inhibitors (HDI): Voninostat (sold under the tradename Zolinza® by Merck);
Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BíCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chlorambucil (sold under the tradename Leukeran®), cyclophosphamide (sold under the tradenames Cytoxan® and Neosar®), dacarbazine (also known as DTIC, DIC and imidazole carboxamide, sold under the tradename DTIC-Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®;
Biologic response modifiers: bacillus calmette-guerin (sold under the tradenames theraCys® and TICE® BCG), denileukin diftitox (sold under the tradename Ontak®);
Anti-tumor antibiotics: doxorubicin (sold under the tradenames Adriamycin® and Rubex®), bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), mitomycin C (sold under the tradename Mutamycin®);
Anti-microtubule agents: Estramustine (sold under the tradename Emcyl®);
Cathepsin K inhibitors: Odanacatib (also known as MK-0822, N-(1-cyanocyclopropyl)-4-fluoro-N2-{(1S)-2,2,2-trifluoro-1-[4′-(methylsulfonyl)biphenyl-4-ylethyl}-L-leucinamide, available from Lanzhou Chon Chemicals, ACC Corp., and ChemieTek, and described in PCT Publication no. WO 03/075836);
Epothilone B analogs: Ixabepilone (sold under the tradename Lxempra® by Bristol-Myers Squibb);
Heat Shock Protein (HSP) inhibitors: Tanespimycin (17-allylamino-17-demethoxygeldanamycin, also known as KOS-953 and 17-AAG, available from SIGMA, and described in U.S. Pat. No. 4,261,989), NVP-HSP990, AUY922, AT13387, STA-9090, Debio 0932, KW-2478, XL888, CNF2024, TAS-116;
TpoR agonists: Eltrombopag (sold under the tradenames Promacta® and Revolade® by GlaxoSmithKline);
Anti-mitotic agents: Docetaxel (sold under the tradename Taxotere® by Sanofi-Aventis);
Adrenal steroid inhibitors: aminoglutethimide (sold under the tradename Cytadren®); Anti-androgens: Nilutamide (sold under the tradenames Nilandron® and Anandron®), bicalutamide (sold under tradename Casodex®), flutamide (sold under the tradename Fulexin™); Androgens: Fluoxymesterone (sold under the tradename Halotestin®);
CDK (CDK1, CDK2, CDK3, CDK5, CDK7, CDK8, CDK9, CDK11/12, or CDK16) inhibitors including but not limited to Alvocidib (pan-CDK inhibitor, also known as flovopirdol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002);
CDK4/6 inhibitors including but not limited to pabociclib, ribociclib, abemaciclib, and Trilaciclib; CDK9 inhibitors including but not limited to AZD 4573, P276-00, AT7519M, TP-1287;
CDK2/4/6 inhibitor such as PF-06873600;
Cell division cycle 25A (CDC25A) inhibitors such as 2-(2-mercaptoethanol)-3-methyl-1,4-naphthoquinone, 1-([1,1′-biphenyl]-4-yl)-3,4-bis((2-hydroxyethyl)thio-1H-pyrrole-2,5-dione (PM-20), 2-(2,5-difluourophenyl)-6-((3-(methyl(3-((2-methyl-4,7-dioxo-4,7-dihydrobenzo[d]-thiazol-5-yl)amino) propyl)amino) propyl)amino)benzo[d]oxazole-4,7-dione (IRC 083864), or 2-methoxyestadiol, or a pharmaceutically acceptable salt thereof;
SHP-2 inhibitor such as TNO155;
MDM2/MDMX, MDM2/p53 and/or MDMX/p53 modulators;
Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);
Taxane anti-neoplastic agents: Cabazitaxel (1-hydroxy-7,10-dimethoxy-9-oxo-5,20-epoxytax-11-ene-2a,4,13a-triyl-4-acetate-2-benzoate-13-[(2R,3S)-3-{[(tert-butoxy)carbonyl]-amino}-2-hydroxy-3-phenylpropanoate), larotaxel ((2α,3ξ,4α,5β,7α,10β,13α)-4,10-bis(acetyloxy)-13-({(2R,3S)-3-[(tert-butoxycarbonyl)amino]-2-hydroxy-3-phenylpropanoyl}-oxy)-1-hydroxy-9-oxo-5,20-epoxy-7,19-cyclotax-11-en-2-yl benzoate);
5HT1a receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573); HPC vaccines: Cervarix® sold by GlaxoSmithKline, Gardasil® sold by Merck; Iron Chelating agents: Deferasinox (sold under the tradename Exjade® by Novartis);
Anti-metabolites: Claribine (2-chlorodeoxyadenosine, sold under the tradename Leustatin®), 5-fluorouracil (sold under the tradename Adrucil®), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatin™), methotrexate (also known as amethopterin, methotrexate sodim (MTX), sold under the tradenames Rheumatrex® and Trexall™), pentostatin (sold under the tradename Nipent®);
Bisphosphonates: Pamidronate (sold under the tradename Aredia®), zoledronic acid (sold under the tradename Zometa®); Demethylating agents: 5-azacitidine (sold under the tradename Vidaza (D), decitabine (sold under the tradename Dacogen®);
Plant Alkaloids: Paclitaxel protein-bound (sold under the tradename Abraxane vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), paclitaxel (sold under the tradenames Taxol and Onxal™);
Retinoids: Alitretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem@, Claravis®, Clarus®, Decutan®, Isotane®, Izotech®, Oratane®, Isotret®, and Sotret®), bexarotene (sold under the tradename Targretin®);
Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®), Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone® Medrol®, M-Prednisol® and Solu-Medrol®);
Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®); Estrogen receptor downregulators: Fulvestrant (sold under the tradename Faslodex®);
Anti-estrogens: tamoxifen (sold under the tradename Novaldex®); Toremifene (sold under the tradename Fareston®);
Selective estrogen receptor modulators (SERMs): Raloxifene (sold under the tradename Evista®);
Leutinizing hormone releasing hormone (LHRH) agonists: Goserelin (sold under the tradename Zoladex®); Progesterones: megestrol (also known as megestrol acetate, sold under the tradename Megace®);
Miscellaneous cytotoxic agents: Arsenic trioxide (sold under the tradename Trisenox®), asparaginase (also known as L-asparaginase, Erwinia L-asparaginase, sold under the tradenames Elspar® and Kidrolaset®);

One or more immune checkpoint inhibitors CD27, CD28, CD40, CD122, CD96, CD73, CD39, CD47, OX40, GITR, CSF1R, JAK, PI3K delta, PI3K gamma, TAM kinase, arginase, CD137 (also known as 4-1BB), ICOS, A2AR, A2BR, HIF-2α, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, OX40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA, In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab, or pembrolizumab or PDR001. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab).

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of OX40, e.g., an anti-OX40 antibody or OX40L fusion protein. In some embodiments, the anti-OX40 antibody is MEDI0562 or, INCAGN01949, GSK2831781, GSK-3174998, MOXR-0916, PF-04518600 or LAG525. In some embodiments, the OX40L fusion protein is MED16383.

Compounds of the invention can also be used to increase or enhance an immune response, including increasing the immune response to an antigen; to improve immunization, including increasing vaccine efficacy; and to increase inflammation. In some embodiments, the compounds of the invention can be sued to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists.

Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of Formula (I) can also be used in combination with the following adjunct therapies: anti-nausea drugs: NK-1 receptor antagonists: Casopitant (sold under the tradenames Rezonic® and Zunrisa® by GlaxoSmithKline); and Cytoprotective agents: Amifostine (sold under the tradename Ethyol®), leucovorin (also known as calcium leucovorin, citrovorum factor and folinic acid).

### EXAMPLES

The following preparations of intermediates and compounds of Formula (I) are given to enable those skilled in the art to more clearly understand and to practice the present disclosure. They should not be considered as limiting the scope of the disclosure, but merely as being illustrative and representative thereof.

### Intermediate 1:

### Synthesis of (3aR,5s,6aS)-5-aminohexahydro-1H-cyclopenta[c]thiophene 2,2-dioxide

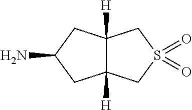 [SMILES: N[C@H]1C[C@@H]2CS(=O)(=O)C[C@@H]2C1]

### Step 1: Dimethyl 4-oxocyclopentane-1,2-dicarboxylate

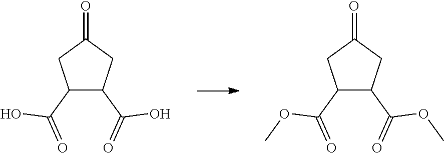

SOCl₂ (7.14 g, 60.00 mmol, 2.00 eq) was added dropwise at room temperature to a solution of 4-oxocyclopentane-1,2-dicarboxylic acid (5.16 g, 30.00 mmol, 1.00 eq) in MeOH (100.0 mL). The mixture was heated to 70° C. for 16 h and then concentrated. The residue was dissolved in EtOAc, washed with H₂O and brine. The organic layer was dried over Na₂SO₄, filtered and concentrated to obtain the title compound as a colorless oil.

### Step 2: Dimethyl 1,4-dioxaspiro[4.4]nonane-7,8-dicarboxylate

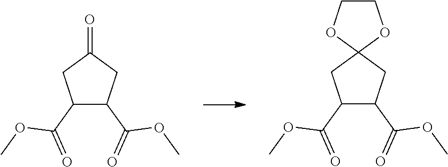

A solution of dimethyl 4-oxocyclopentane-1,2-dicarboxylate (4.70 g, 23.50 mmol, 1.00 eq), ethane-1,2-diol (1.75 g, 28.20 mmol, 1.20 eq) and TsOH·H₂O (200 mg, cat.) in toluene (100.0 mL) was heated to 140° C. for 16 h with Dean-Stark apparatus. Then the mixture was concentrated and purified with silica gel column chromatography (PE/EtOAc=5:1) to obtain the title compound as a colorless oil.

### Step 3:1,4-Dioxaspiro[4.4]nonane-7,8-diyldimethanol

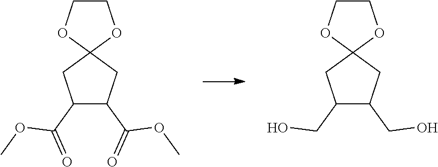

A solution of LiAlH₄ (690 mg, 18.20 mmol, 1.20 eq) in THF (20.0 mL) was cooled to 0° C., and dimethyl 1,4-dioxaspiro[4.4]nonane-7,8-dicarboxylate (3.70 g, 15.20 mmol, 1.00 eq) in THF (30.0 mL) was added dropwise at 0° C. The mixture was allowed to stir at 0° C. for 1 hour before it was quenched with H₂O. The mixture was filtered, and the filter cake was washed with MeOH. The filtrate was combined and concentrated to obtain the title compound as a yellow oil.

### Step 4:1,4-Dioxaspiro[4.4]nonane-7,8-diylbis(methylene) dimethanesulfonate

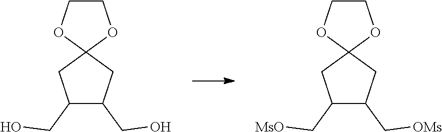 [SMILES: OCC1CC2(CC1CO)OCCO2 ; CS(=O)(=O)OCC1CC2(CC1COS(C)(=O)=O)OCCO2]

TEA (8.06 g, 79.80 mmol, 6.00 eq) was added to a solution of 1,4-dioxaspiro[4.4]nonane-7,8-diyldimethanol (2.50 g, 13.30 mmol, 1.00 eq) in DCM (30 mL), followed by addition of MsCl (4.55 g, 39.90 mmol, 3.00 eq) dropwise at room temperature. The mixture was stirred at room temperature for 5 h before it was poured onto ice. The mixture was extracted with DCM. The organic layer was combined and concentrated to obtain the title compound as a yellow oil.

### Step 5: (3aR,6aS)-Tetrahydro-1H,3H-spiro[cyclopenta[c]thiophene-5,2′-[1,3]dioxolane]

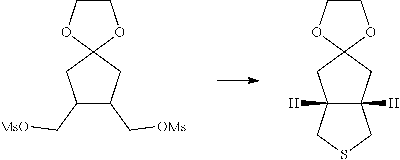 [SMILES: CS(=O)(=O)OCC1CC2(CC1COS(C)(=O)=O)OCCO2 ; C1COC2(C[C@H]3CSC[C@H]3C2)O1]

Na₂S (907 mg, 11.60 mmol, 1.00 eq) was added to a solution of 1,4-dioxaspiro-[4.4]nonane-7,8-diylbis(methylene) dimethanesulfonate (4.00 g, 11.60 mmol, 1.00 eq) in EtOH (30.0 mL) at room temperature. The mixture was heated to 60° C. for 16 h under Na atmosphere before it was poured into water. The mixture was extracted with EtOAc. The organic layers were combined, washed with brine, dried over Na₂SO₄, concentrated and the residue was purified with silica gel column chromatography (PE/EtOAc=10:1) to obtain the title compound as a white solid.

### Step 6: (3aR,6aS)-Tetrahydro-1H-cyclopenta[c]thiophen-5 (3H)-one

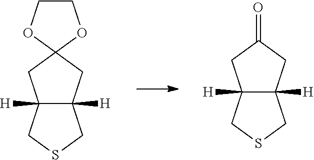 [SMILES: C1COC2(C[C@H]3CSC[C@H]3C2)O1 ; O=C1C[C@H]2CSC[C@H]2C1]

2 M HCl aq. solution (5.0 mL) was added to a mixture of (3aR,6aS)-tetrahydro-1H,3H-spiro[cyclopenta[c]thiophene-5,2′-[1,3]dioxolane] (750 mg, 4.03 mmol, 1.00 eq) in acetone (5.0 mL). The mixture was stirred at room temperature for 16 h, diluted with H₂O. The mixture was extracted with EtOAc and the organic layers were combined, washed with sat. NaHCO₃ aq. solution and brine, dried with Na₂SO₄, filtered and concentrated. The residue was purified with silica gel column chromatography (PE/EtOAc=10:1) to obtain the title compound as a yellow solid.

### Step 7: (3aR,5r,6aS)-Hexahydro-1H-cyclopenta[c]thiophen-5-ol

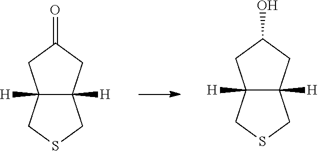 [SMILES: O=C1C[C@H]2CSC[C@H]2C1 ; O[C@H]1C[C@H]2CSC[C@H]2C1]

A solution of (3aR,6aS)-tetrahydro-1H-cyclopenta[c]thiophen-5 (3H)-one (400 mg, 2.81 mmol, 1.00 eq) in MeOH (10.0 mL) was added NaBH₄ (321 mg, 8.45 mmol, 3.00 eq) portion-wise. The mixture was stirred at room temperature for 3 h, diluted with H₂O, extracted with EtOAc. The organic layer was combined, washed with brine, dried with Na₂SO₄, filtered and concentrated to obtain the title compound as a yellow oil.

### Step 8: (3aR,5r,6aS)-Hexahydro-1H-cyclopenta[c]thiophen-5-yl methanesulfonate

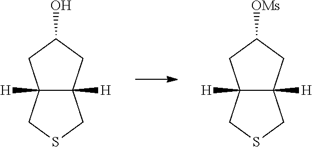 [SMILES: O[C@H]1C[C@H]2CSC[C@H]2C1 ; CS(=O)(=O)O[C@H]1C[C@H]2CSC[C@H]2C1]

TEA (840 mg, 8.33 mmol, 3.00 eq) was added to a solution of (3aR,5r,6aS)-hexahydro-1H-cyclopenta[c]thiophen-5-ol (400 mg, 2.78 mmol, 1.00 eq) in DCM (10.0 mL) followed by addition of MsCl (380 mg, 3.33 mmol, 1.20 eq) dropwise at room temperature. The mixture was stirred 3 h before it was poured into water (10.0 mL), and the mixture was extracted with DCM. The organic layers were combined, washed with brine, dried with Na₂SO₄, filtered, and concentrated to obtain the title compound as a yellow oil.

### Step 9: (3aR,5s,6aS)-Hexahydro-1H-cyclopenta[c]thiophen-S-amine

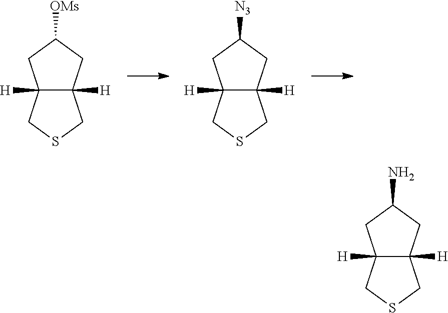 [SMILES: CS(=O)(=O)O[C@H]1C[C@H]2CSC[C@H]2C1 ; [N-]=[N+]=N[C@@H]1C[C@H]2CSC[C@H]2C1 ; N[C@@H]1C[C@H]2CSC[C@H]2C1]

NaN₃ (350 mg, 5.40 mmol, 2.00 eq) was added to a solution of (3aR,5r,6aS)-hexahydro-1H-cyclopenta[c]thiophen-5-yl methanesulfonate (600 mg, 2.70 mmol, 1.00 eq) in DMF (6 mL). The mixture was heated to 60° C. 16 h under Na atmosphere, then it was diluted with EtOAc and H₂O. The organic layers were combined, concentrated. The residue was diluted with NH; in MeOH (7 M, 10 mL), followed by addition of Pd/C (10%, 120 mg, 0.2 W/W). The mixture was stirred at room temperature 16 h under Ha atmosphere before it was filtered and concentrated. The residue was purified by silica gel column chromatography (DCM/MeOH=10:1) to obtain the title compound as a yellow oil.

### Step 10: tert-Butyl (3aR,5s,6aS)-2,2-dioxidohexahydro-1H-cyclopenta[c]thiophen-5-yl)-carbamate

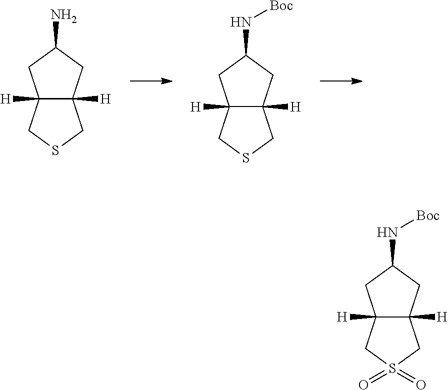 [SMILES: N[C@@H]1C[C@H]2CSC[C@H]2C1 ; CC(C)(C)OC(=O)N[C@@H]1C[C@H]2CSC[C@H]2C1 ; CC(C)(C)OC(=O)N[C@H]1C[C@@H]2CS(=O)(=O)C[C@@H]2C1]

(Boc)₂O) (685 mg, 3.15 mmol, 1.50 eq) was added to a solution of (3aR,5s,6aS)-hexahydro-1H-cyclopenta[c]thiophen-5-amine (300 mg, 2.10 mmol, 1.00 eq) in THF (10.0 mL) dropwise. The mixture was stirred at room temperature 2 h before it was diluted with H₂O and extracted with EtOAc. The organic layers were combined, washed with brine, and concentrated under reduced pressure. The residue was dissolved in THF/H₂O (4:1, 10 mL), followed by addition of oxone (2.58 g, 4.20 mmol, 2.00 eq). The mixture was stirred at room temperature 16 h before it was diluted with H₂O and extracted with EtOAc. The organic layers were combined, concentrated and purified by silica gel column chromatography (PE/EtOAc=10:1) to obtain the title compound as a yellow solid.

### Step 11: (3aR,5s,6aS)-5-Aminohexahydro-1H-cyclopenta[c]thiophene 2,2-dioxide

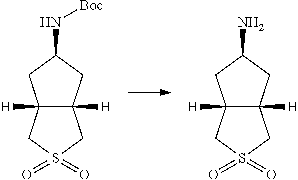 [SMILES: CC(C)(C)OC(=O)N[C@H]1C[C@@H]2CS(=O)(=O)C[C@@H]2C1 ; N[C@H]1C[C@@H]2CS(=O)(=O)C[C@@H]2C1]

HCl in MeOH (4 M, 5.0 mL) was added to tert-butyl ((3aR,5s,6aS)-2,2-dioxidohexahydro-1H-cyclopenta[c]thiophen-5-yl) carbamate (450 mg, 1.64 mmol, 1.00 eq) at room temperature. After stirring for 3 h, the mixture was concentrated. The residue was suspended in DCM, followed by addition of aq. NaHCO₃. The mixture was stirred at room temperature 1 h, then it was filtered. The layers were separated, and the organic layer was concentrated to obtain the title compound.

### Intermediate 2:

### Synthesis of (1R,5S,6s)-6-amino-3-thiabicyclo[3.1.0]hexane 3,3-dioxide

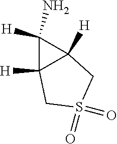

### Step 1: Ethyl (1R,5S,6r)-3-thiabicyclo[3.1.0]hexane-6-carboxylate 3,3-dioxide and ethyl (1R,5S,6s)-3-thiabicyclo[3.1.0]hexane-6-carboxylate 3,3-dioxide

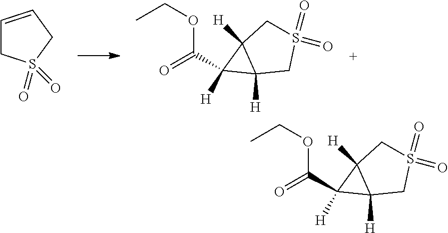

Diazoacetic acid ethyl ester (7.20 g, 63.48 mmol, 1.5 eq) in DCM (50.0 mL) was added slowly via syringe pump over 5 h to a stirred solution of 2,5-dihydro-thiophene 1,1-dioxide (5.00 g, 42.32 mmol, 1.00 eq) and Rhodium (II) acetate (281 mg, 1.27 mmol, 0.03 eq) in DCM (80.0 mL) under N₂ at rt. The mixture was concentrated and the residue was purified by column chromatography on silica gel (PE:EA=5:1) to separate the title compounds.

### Step 2: (1R,5S,6r)-3-Thiabicyclo[3.1.0]hexane-6-carboxylic acid 3,3-dioxide

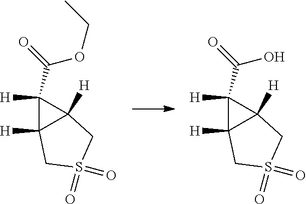

A solution of LiOH·H₂O (740 mg, 17.63 mmol, 3.00 eq) in H₂O (6.0 mL) was added to a mixture of ethyl (1R,5S,6r)-3-thiabicyclo[3.1.0]hexane-6-carboxylate 3,3-dioxide (1.2 g, 5.88 mmol, 1.00 eq) in EtOH (12.0 mL). This mixture was stirred at rt. 3 h. pH of the mixture was adjusted to 2-3 with 1N HCl, and the mixture was extracted with DCM. The combined organic layers were concentrated to give the crude product as a pale-yellow solid.

### Step 3: Benzyl ((1R,5S,6s)-3,3-dioxido-3-thiabicyclo[3.1.0]hexan-6-yl)carbamate

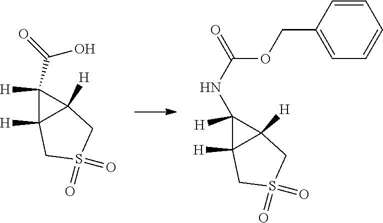 [SMILES: O=C(O)[C@@H]1[C@@H]2CS(=O)(=O)C[C@@H]21 ; O=C(N[C@@H]1[C@@H]2CS(=O)(=O)C[C@@H]21)OCc1ccccc1]

A mixture of (1R,5S,6r)-3-thiabicyclo[3.1.0]hexane-6-carboxylic acid 3,3-dioxide (500 mg, 2.84 mmol, 1.00 eq) in toluene (15.0 mL) was added TEA (459 mg, 4.54 mmol, 1.60 eq) and DPPA (937 mg, 3.41 mmol, 1.20 eq. This mixture was stirred at rt. under N₂ for 2 h and then benzyl alcohol (614 mg, 5.68 mmol, 2.00 eq) was added dropwise. This mixture was stirred at 100° C. under N₂ overnight before it was concentrated and purified by column chromatography on silica gel (PE:EA=2:1) to give the title compound as a white solid.

### Step 4: (1R,5S,6s)-6-Amino-3-thiabicyclo[3.1.0]hexane 3,3-dioxide

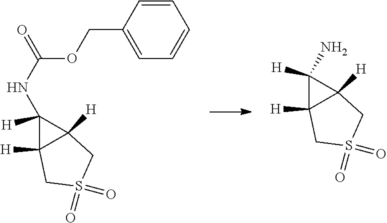 [SMILES: O=C(N[C@@H]1[C@@H]2CS(=O)(=O)C[C@@H]21)OCc1ccccc1 ; N[C@H]1[C@H]2CS(=O)(=O)C[C@@H]12]

A mixture of benzyl ((1R,5S,6s)-3,3-dioxido-3-thiabicyclo[3.1.0]hexan-6-yl) carbamate (500 mg, 0.36 mmol, 1.00 eq) in MeOH (5.0 mL) was added Pd/C (50 mg). This mixture was stirred at rt. under H₂ for 5 h. The mixture was filtered, concentrated to give the title compound as a white solid.

### Intermediate 3:

### Synthesis of (1R,5S,6r)-6-amino-3-thiabicyclo[3.1.0]hexane 3,3-dioxide

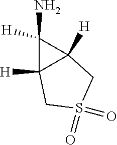

The title compound was prepared by proceeding analogously as described for Intermediate 2 with ethyl (1R,5S,6s)-3-thiabicyclo[3.1.0]hexane-6-carboxylate 3,3-dioxide replacing ethyl (1R,5S,6r)-3-thiabicyclo[3.1.0]hexane-6-carboxylate 3,3-dioxide in Step 2-4.

### Intermediate 4:

### Synthesis of (1s,4s)-4-(methylsulfonyl)cyclohexan-1-amine trifluoroacetate

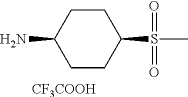

### Step 1: (1r,4r)-4-((tert-Butoxycarbonyl)amino)cyclohexyl methanesulfonate

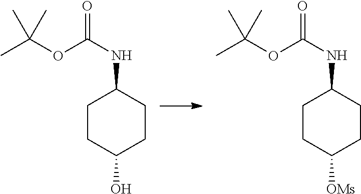

TEA (4.20 g, 41.79 mmol, 3.00 eq.) was added to a mixture of tert-butyl ((1r,4r)-4-hydroxycyclohexyl) carbamate (3.00 g, 13.93 mmol, 1.00 eq.) in DCM (30.0 mL), followed by addition of MsCl (2.40 g, 20.90 mmol, 1.50 eq.) slowly at 0° C. This mixture was stirred at 0° C. under N₂ 1 h and then it was warmed to rt. and stirred under N₂ 2 h. The mixture was diluted with H₂O and extracted with DCM. The combined organic layers were washed with brine, dried over Na₂SO₄, concentrated. The residue was purified by column chromatography on silica gel (PE:EA=5:1) to give the title compound as a white solid.

### Step 2: tert-Butyl ((1s,4s)-4-(methylthio)cyclohexyl) carbamate

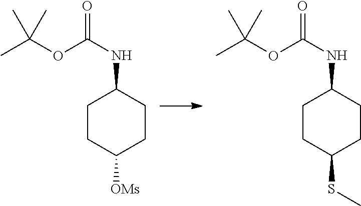

CH₃SNa (1.10 g, 15.68 mmol, 2.00 eq.) was added to a mixture of (1r,4r)-4-((tert-butoxy-carbonyl)amino)cyclohexyl methanesulfonate (2.30 g, 7.84 mmol, 1.00 eq.) in DMF (23.0 mL). This mixture was stirred at rt overnight and then diluted with H₂O and extracted with EtOAc. The combined organic layers were washed with brine, dried over Na₂SO₄, and concentrated. The residue was purified by silica gel column chromatography (PE:EA=10:1) to give the title compound as a white solid.

### Step 3: tert-Butyl ((1s,4s)-4-(methylsulfonyl)cyclohexyl) carbamate

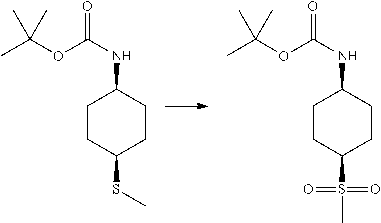 [SMILES: CS[C@H]1CC[C@@H](NC(=O)OC(C)(C)C)CC1 ; CC(C)(C)OC(=O)N[C@H]1CC[C@@H](S(C)(=O)=O)CC1]

To a mixture of tert-butyl ((1s,4s)-4-(methylthio)cyclohexyl) carbamate (202 mg, 0.82 mmol, 1.00 eq.) in THF/MeOH/H₂O (2:2:1, 4.0 mL) was added oxone (504 mg, 1.64 mmol, 2.00 eq.) and the resulting mixture was stirred at r.t. under N₂ overnight. H₂O was added and the mixture was extracted with EtOAc, washed with brine, dried over Na₂SO₄, and concentrated to give the crude product as a pale-yellow solid.

### Step 4: (1s,4s)-4-(Methylsulfonyl)cyclohexan-1-amine trifluoroacetate

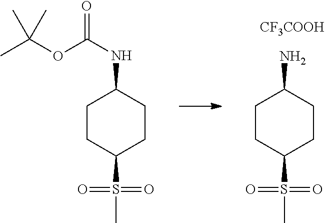 [SMILES: CC(C)(C)OC(=O)N[C@H]1CC[C@@H](S(C)(=O)=O)CC1 ; "CF3COOH" ; CS(=O)(=O)[C@H]1CC[C@@H](N)CC1]

TFA (0.5 mL) was added to a mixture of tert-butyl ((1s,4s)-4-(methylsulfonyl)-cyclohexyl) carbamate (100 mg, 0.36 mmol, 1.00 eq) in DCM (2.0 mL). The mixture was stirred at rt for 2 h before it was concentrated to give the crude title compound as a yellow oil.

### Intermediate 5:

### Synthesis of (1r,4r)-4-(methylsulfonyl)cyclohexan-1-amine trifluoroacetate

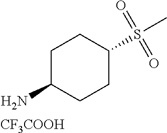

The title compound was prepared by proceeding analogously as described in Intermediate 4 by replacing tert-butyl ((1s,4s)-4-hydroxycyclohexyl) carbamate for tert-butyl ((1r,4r)-4-hydroxycyclohexyl) carbamate in Step 1.

### Intermediate 6:

### Synthesis of 6-amino-2-thiaspiro[3.3]heptane 2,2-dioxide

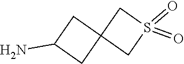

### Step 1: Diisopropyl 3-oxocyclobutane-1,1-dicarboxylate

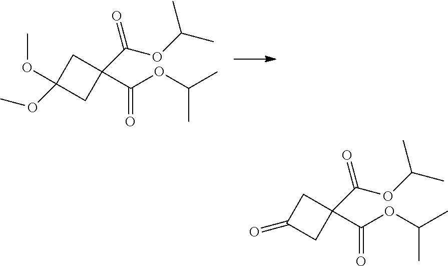 [SMILES: COC1(OC)CC(C(=O)OC(C)C)(C(=O)OC(C)C)C1 ; CC(C)OC(=O)C1(C(=O)OC(C)C)CC(=O)C1]

To a mixture of diisopropyl 3,3-dimethoxycyclobutane-1,1-dicarboxylate (40.00 g, 139.00 mmol) in acetone (100.0 ml) was added 2N HCl aq. (100.0 mL) and the mixture was stirred at rt overnight. The reaction mixture was diluted with EA, washed with water, sat. NaHCO₃ aq., and brine. The organic layer was dried over Na₂SO₄, filtered, and concentrated to give crude product as a yellow liquid.

### Step 2: Diisopropyl 3-hydroxycyclobutane-1,1-dicarboxylate

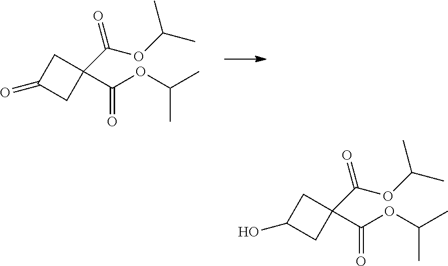 [SMILES: CC(C)OC(=O)C1(C(=O)OC(C)C)CC(=O)C1 ; CC(C)OC(=O)C1(C(=O)OC(C)C)CC(O)C1]

NaBH₄ (15.70 g, 416.10 mmol, 3.00 eq) was added to a mixture of diisopropyl 3-oxocyclobutane-1,1-dicarboxylate (33.60 g, 138.70 mmol, 1.00 eq) in IPA (300.0 ml) and the mixture was stirred at r.t. overnight. The mixture was diluted with water and extracted with EtOAc. The combined organic layers were washed with water, brine, dried over Na₂SO₄, and concentrated to give the crude product as a yellow oil.

### Step 3: Diisopropyl 3-((methylsulfonyl)oxy)cyclobutane-1,1-dicarboxylate

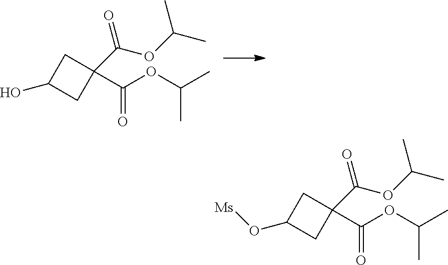 [SMILES: CC(C)OC(=O)C1(C(=O)OC(C)C)CC(O)C1 ; CC(C)OC(=O)C1(C(=O)OC(C)C)CC(OS(C)(=O)=O)C1]

To a mixture of diisopropyl 3-hydroxycyclobutane-1,1-dicarboxylate (28.0 g, 115.0 mmol, 1.00 eq) in DCM (300.0 ml) was added TEA (34.80 g, 344.0 mmol, 3.0 eq), followed by addition of MsCl (15.8 g, 138.0 mmol, 1.2 eq) and the mixture was stirred at rt 3 h. The mixture was diluted with water and extracted with DCM. The combined organic layers were washed with brine, dried over Na₂SO₄, filtered, and concentrated to give crude product as a yellow oil.

### Step 4: Diisopropyl 3-azidocyclobutane-1,1-dicarboxylate

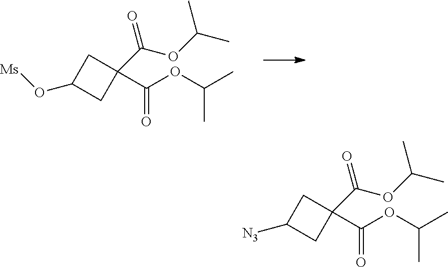 [SMILES: CC(C)OC(=O)C1(C(=O)OC(C)C)CC(OS(C)(=O)=O)C1 ; CC(C)OC(=O)C1(C(=O)OC(C)C)CC(N=[N+]=[N-])C1]

NaN₃ (14.90 g, 229.00 mmol, 2.00 eq) was added to a mixture of diisopropyl 3-((methylsulfonyl)oxy)cyclobutane-1,1-dicarboxylate (36.95 g, 115.0 mmol, 1.00 eq) in DMF (200.0 ml), and the mixture was stirred at 90° C. overnight under N₂. The mixture was diluted with water and extracted with EtOAc. The combined organic layers were washed with water, brine, dried over Na₂SO₄, filtered, concentrated, and the residue was purified by silica gel column chromatography (PE:BA=20:1) to give the title compound as a yellow liquid.

### Step 5: Diisopropyl 3-aminocyclobutane-1,1-dicarboxylate

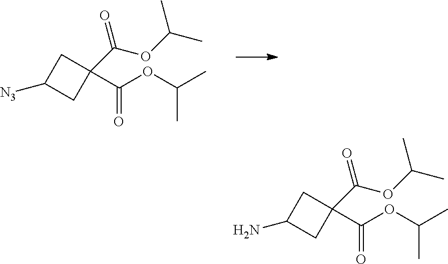 [SMILES: CC(C)OC(=O)C1(C(=O)OC(C)C)CC(N=[N+]=[N-])C1 ; CC(C)OC(=O)C1(C(=O)OC(C)C)CC(N)C1]

10% Pd/C (3.00 g) was added to a mixture of diisopropyl 3-azidocyclobutane-1,1-dicarboxylate (15.00 g, 55.70 mmol, 1.00 eq) in IPA/NHs (150.0 mL), and the mixture was allowed to stir at r.t. overnight under H₂. The mixture was filtered and the filtrate was concentrated and purified by silica gel column chromatography (PE:EA=1:1 to DCM:MeOH=20:1) to give the title compound as a yellow oil.

### Step 6: Diisopropyl 3-((tert-butoxycarbonyl)amino)cyclobutane-1,1-dicarboxylate

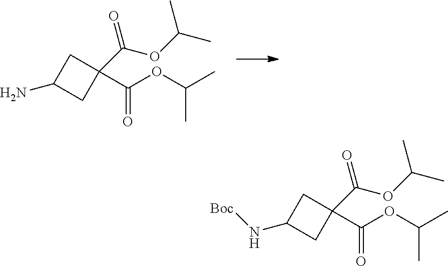 [SMILES: CC(C)OC(=O)C1(C(=O)OC(C)C)CC(N)C1 ; CC(C)OC(=O)C1(C(=O)OC(C)C)CC(NC(=O)OC(C)(C)C)C1]

Boc₂O (10.8 g, 49.4 mmol, 1.20 eq) was added to a mixture of diisopropyl 3-amino-cyclobutane-1,1-dicarboxylate (10.0 g, 41.10 mmol, 1.00 eq) in DCM (100.0 ml) and the mixture was stirred at r.t. overnight. The reaction mixture was washed with 1N HCl aq., and brine. The organic layer was dried over Na₂SO₄, filtered, and concentrated to give crude product as a yellow liquid.

### Step 7: tert-Butyl (3,3-bis(hydroxymethyl)cyclobutyl)carbamate

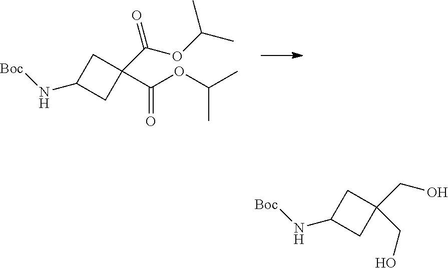 [SMILES: CC(C)OC(=O)C1(C(=O)OC(C)C)CC(NC(=O)OC(C)(C)C)C1 ; CC(C)(C)OC(=O)NC1CC(CO)(CO)C1]

Diisopropyl 3-((tert-butoxycarbonyl)amino)cyclobutane-1,1-dicarboxylate (14.00 g, 40.80 mmol, 1.00 eq) in THF (50.0 mL) was added to LiAlH₄ (1.70 g, 44.90 mmol, 1.10 eq) in THF (150.0 ml) dropwise at 0° C. The resulting mixture was warmed to rt and stirred for 3 h and then quenched by water. After stirring for 30 min., the mixture was filtered, and the organic layer was concentrated to give crude product as a yellow oil.

### Step 8: (3-((tert-Butoxycarbonyl)amino)cyclobutane-1,1-diyl)bis(methylene) dimethanesulfonate

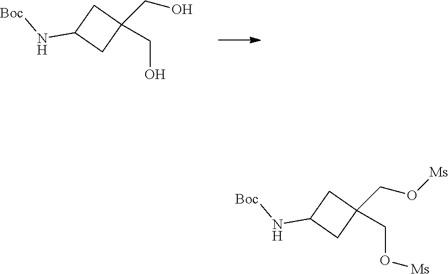 [SMILES: CC(C)(C)OC(=O)NC1CC(CO)(CO)C1 ; CC(C)(C)OC(=O)NC1CC(COS(C)(=O)=O)(COS(C)(=O)=O)C1]

To a mixture of tert-butyl (3,3-bis(hydroxymethyl)cyclobutyl) carbamate (9.40 g, 40.60 mmol, 1.00 eq) and TEA (24.7 g, 243.8 mmol, 6.0 eq) in DCM (120.0 ml) was added MsCl (14.0 g, 121.9 mmol, 3.00 eq) and the mixture was stirred at rt for 3 h. The mixture was poured into ice cold water and extracted with DCM. The combined organic layers were washed with brine, dried over Na₂SO₄, and concentrated to give crude product as a yellow oil.

### Step 9: tert-Butyl 2-thiaspiro[3.3]heptan-6-ylcarbamate

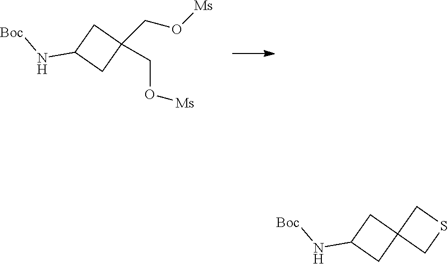 [SMILES: CC(C)(C)OC(=O)NC1CC(COS(C)(=O)=O)(COS(C)(=O)=O)C1 ; CC(C)(C)OC(=O)NC1CC2(CSC2)C1]

Na₂S (3.20 g, 40.50 mmol, 1.00 eq) was added to a mixture of (3-((tert-butoxycarbonyl)-amino)cyclobutane-1,1-diyl)bis(methylene) dimethanesulfonate (15.70 g, 40.50 mmol, 1.00 eq) in EtOH (100.0 ml) and the mixture was stirred at 60° C. overnight under N₂. The mixture was poured into ice cold water and extracted with EtOAc. The combined organic layers were washed with brine, dried over Na₂SO₄, concentrated, and the residue was purified by column chromatography on silica gel (PE:EA=10:1) to give the title compound as a yellow solid.

### Step 10: tert-Butyl (2,2-dioxido-2-thiaspiro[3.3]heptan-6-yl) carbamate

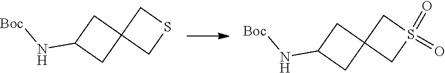 [SMILES: CC(C)(C)OC(=O)NC1CC2(CSC2)C1 ; CC(C)(C)OC(=O)NC1CC2(C1)CS(=O)(=O)C2]

Oxone (22.50 g, 36.70 mmol, 3.00 eq) was added to a mixture of tert-butyl 2-thiaspiro-[3.3]heptan-6-ylcarbamate (2.80 g, 12.20 mmol, 1.00 eq) in MeOH:THF:H₂O=2:2:1 (100.0 mL) and the mixture was stirred at r.t. overnight. The mixture was diluted with H₂O and extracted with EtOAc. The combined organic layers were washed with water, brine, dried over Na₂SO₄, filtered and concentrated to give crude product.

### Step 11: 6-Amino-2-thiaspiro[3.3]heptane 2,2-dioxide

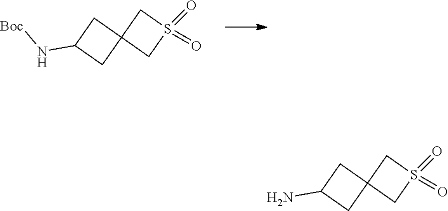 [SMILES: CC(C)(C)OC(=O)NC1CC2(C1)CS(=O)(=O)C2 ; NC1CC2(C1)CS(=O)(=O)C2]

To a mixture of tert-butyl (2,2-dioxido-2-thiaspiro[3.3]heptan-6-yl) carbamate (3.19 g, 12.20 mmol, 1.00 eq) in MeOH (2.0 ml) was added MeOH/HCl (10.0 mL) and the mixture was stirred at r.t. for 2 h. The reaction mixture was concentrated and purified by column chromatography on silica gel (PE:EA=1:1 to DCM:MeOH=10:1) to give the title compound.

### Intermediate 7:

### Synthesis of N-(3-(2-bromo-2-(2-chloropyrimidin-4-yl)acetyl)-2-fluorophenyl)acetamide

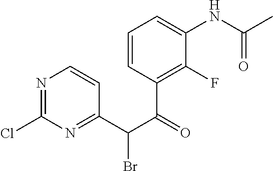 [SMILES: CC(=O)Nc1cccc(C(=O)C(Br)c2ccnc(Cl)n2)c1F]

### Step 1: Methyl 3-acetamido-2-fluorobenzoate

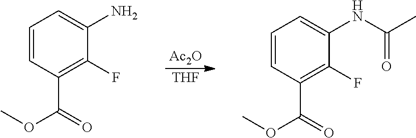

Ac₂O (23.10 g, 0.23 mol, 1.50 eq) was added to a solution of methyl 3-amino-2-fluorobenzoate (25.50 g, 0.15 mol, 1.00 eq) in THF (250.0 mL) and the mixture was stirred at rt overnight under N₂. The mixture was concentrated and the residue was purified by column chromatography on silica gel (PE:EA=5:1 to 3:1) to give the title compound as a pink solid.

### Step 2: N-(3-(2-(2-Chloropyrimidin-4-yl)acetyl)-2-fluorophenyl)acetamide

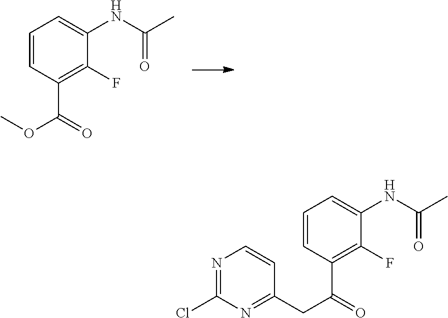 [SMILES: COC(=O)c1cccc(NC(C)=O)c1F ; CC(=O)Nc1cccc(C(=O)Cc2ccnc(Cl)n2)c1F]

LiHMDS (427.0 mL, 0.43 mol, 3.00 eq) in THF was added to a solution of methyl 3-acetamido-2-fluorobenzoate (30.0 g, 0.14 mol, 1.00 eq) in THF (300.0 mL) at 0° C. under N₂, followed by addition of 2-chloro-4-methylpyrimidine (23.70 g, 0.18 mol, 1.30 eq) in THF (100.0 mL) dropwise at 0° C. The mixture was stirred at r.t. for 2 h before it was quenched by aqueous NH₄Cl The mixture was extracted with EtOAc, and the combined organic layers were dried over Na₂SO₄, filtered, concentrated and the residue was purified by column chromatography on silica gel (PE:EA=2:1) to 1:1) to give the title compound as a pink solid.

### Step 3: N-(3-(2-Bromo-2-(2-chloropyrimidin-4-yl)acetyl)-2-fluorophenyl)acetamide

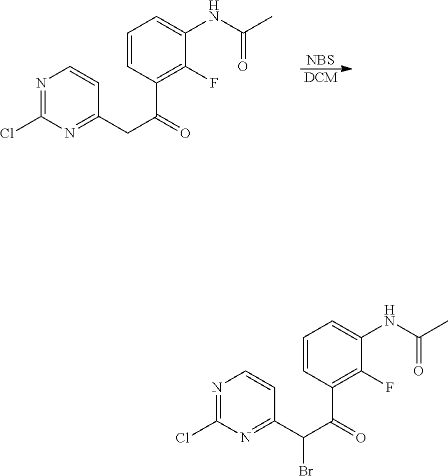

NBS (19.70 g, 0.11 mmol, 1.00 eq) was added to a solution of N-(3-(2-(2-chloropyrimidin-4-yl)acetyl)-2-fluorophenyl)acetamide (34.0 g, 0.11 mol, 1.00 eq) in DCM (350.0 mL) portion-wise and the mixture was stirred at r.t. overnight. The mixture was washed with water, and the organic layer was dried over Na₂SO₄, filtered and concentrated. The residue was purified by column chromatography on silica gel (PE:EA=5:1) to get the title compound as a yellow oil.

### Intermediate 8:

### Synthesis of 4-(difluoromethyl)isoindoline

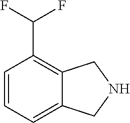 [SMILES: FC(F)c1cccc2c1CNC2]

### Step 1: tert-Butyl 4-bromoisoindoline-2-carboxylate

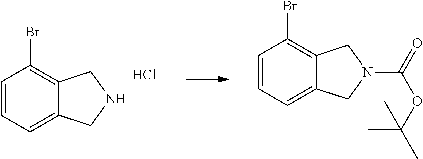 [SMILES: Brc1cccc2c1CNC2 ; Cl ; CC(C)(C)OC(=O)N1Cc2cccc(Br)c2C1]

Boc₂O (5.60 g, 25.64 mmol, 1.50 eq.) was added to a solution of 4-bromoisoindoline hydrochloride (4.00 g, 17.09 mmol, 1.00 eq.) and TEA (5.20 g, 51.28 mmol, 3.00 eq.) in DCM (40.0 mL) and the mixture was stirred at rt overnight. The mixture was concentrated and the residue was purified by column chromatography on silica gel (PE/EA=20:1) to give the title compound as a white solid.

### Step 2: tert-Butyl 4-formylisoindoline-2-carboxylate

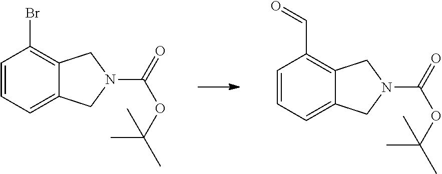 [SMILES: CC(C)(C)OC(=O)N1Cc2cccc(Br)c2C1 ; CC(C)(C)OC(=O)N1Cc2cccc(C=O)c2C1]

n-BuLi (2.5 M in hexane, 3.0 mL, 7.55 mmol, 1.50 eq.) was added to a solution of tert-butyl 4-bromoisoindoline-2-carboxylate (1.50 g, 5.03 mmol, 1.00 eq.) in THF (15.0 mL) at −78° C. under N₂. The mixture was stirred at −78° C. for 30 min, followed by addition of DMF (735 mg, 10.07 mmol, 2.00 eq.) in THF (2.0 mL) dropwise. The mixture was stirred at −78° C. for 2 h and then quenched with NH₄Cl aq., and then diluted with EtOAc. The organic layer was washed with water and brine, dried over Na₂SO₄, filtered and concentrated. The residue was purified by column chromatography on silica gel (PE/EA=10:1) to give the title compound as yellow oil.

### Step 3: tert-Butyl 4-(difluoromethyl)isoindoline-2-carboxylate

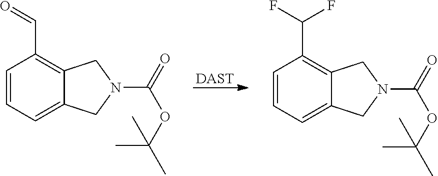

A solution of DAST (587 mg, 0.64 mmol, 5.00 eq.) in DCM (1.0 mL) was added to a solution of tert-butyl 4-formylisoindoline-2-carboxylate (180 mg, 0.73 mmol, 1.00 eq.) in DCM (4.0 mL). The mixture was stirred at rt overnight and then concentrated. The residue was purified by column chromatography on silica gel (PE/EA=10:1) to give the title compound as yellow solid.

### Step 4:4-(Difluoromethyl)isoindoline

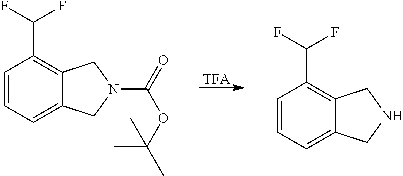 [SMILES: CC(C)(C)OC(=O)N1Cc2cccc(C(F)F)c2C1 ; FC(F)c1cccc2c1CNC2 ; "TFA"]

TFA (1.0 mL) was added to a solution of tert-butyl 4-(difluoromethyl)isoindoline-2-carboxylate (150 mg, 0.36 mmol, 1.00 eq.) in DCM (4.0 mL). The mixture was stirred at rt 3 h and then concentrated to give the title compound as a yellow solid.

### Intermediate 9:

### Synthesis of 6-(difluoromethyl)indoline

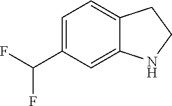 [SMILES: FC(F)c1ccc2c(c1)NCC2]

### Step 1: tert-Butyl 6-formyl-1H-indole-1-carboxylate

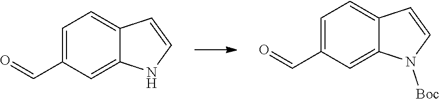 [SMILES: O=Cc1ccc2cc[nH]c2c1 ; CC(C)(C)OC(=O)n1ccc2ccc(C=O)cc21]

Boc₂O (2.20 g, 10.34 mmol, 1.50 eq.) was added to a mixture of 1H-indole-6-carbaldehyde (1.00 g, 6.90 mmol, 1.00 eq.), DMAP (84 mg, 10.69 mmol, 0.10 eq.) and TEA (2.10 g, 20.69 mmol, 3.00 eq.) in DCM (15.0 mL). The mixture was stirred at rt overnight. The organic layer was washed with 1N HCl and brine, dried over Na₂SO₄, filtered, and concentrated to give the title compound as a white solid.

### Step 2: tert-Butyl 6-(difluoromethyl)-1H-indole-1-carboxylate

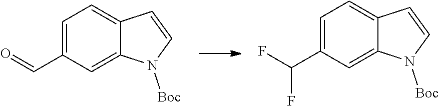 [SMILES: CC(C)(C)OC(=O)n1ccc2ccc(C=O)cc21 ; CC(C)(C)OC(=O)n1ccc2ccc(C(F)F)cc21]

A solution of tert-butyl 6-formyl-1H-indole-1-carboxylate (1.10 g, 4.49 mmol, 1.00 eq.) in DCM (15.0 mL) was added DAST (3.60 g, 22.45 mmol, 5.00 eq.) in DCM (5.0 mL) dropwise at 0° C. and stirred overnight. The mixture was diluted with DCM, washed with NaHCO₃ aq, and brine, dried over Na₂SO₄, filtered, and concentrated. The residue was purified by column chromatography on silica gel (PE/EA=4:1) to give the title compound as yellow oil.

### Step 3: tert-Butyl 6-(difluoromethyl)indoline-1-carboxylate

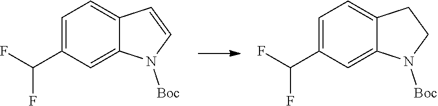 [SMILES: CC(C)(C)OC(=O)n1ccc2ccc(C(F)F)cc21 ; CC(C)(C)OC(=O)N1CCc2ccc(C(F)F)cc21]

A mixture of tert-butyl 6-(difluoromethyl)-1H-indole-1-carboxylate (500 mg, 1.87 mmol, 1.00 eq.) and Pd/C (10%, 100 mg, 20% wt) in MeOH (15.0 mL) was stirred at 50° C. for 3 h. The mixture was filtered, and the filtrate was concentrated. The residue was purified by column chromatography on silica gel PE/EtOAc (3:1) to give the title compound as yellow solid.

### Step 4: 6-(Difluoromethyl)indoline

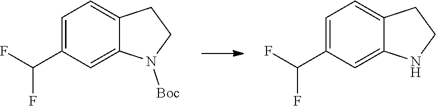 [SMILES: CC(C)(C)OC(=O)N1CCc2ccc(C(F)F)cc21 ; FC(F)c1ccc2c(c1)NCC2]

TFA (1.0 mL) was added to a mixture of tert-butyl 6-(difluoromethyl)indoline-1-carboxylate (350 mg, 1.30 mmol, 1.00 eq.) in DCM (4.0 mL) and the mixture stirred at rt for 2 h. The mixture was concentrated to give the title compound as a yellow oil.

### Intermediate 10:

### Synthesis of isoindolin-5-ylmethanol

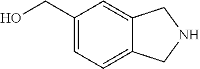 [SMILES: OCc1ccc2c(c1)CNC2]

### Step 1: Methyl 3,4-bis(bromomethyl)benzoate

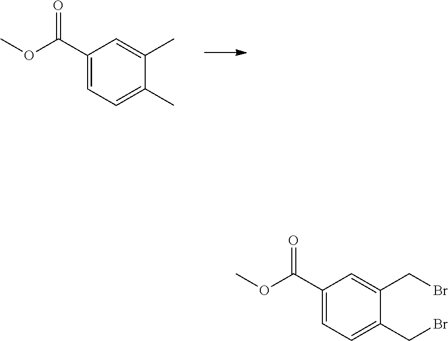

NBS (21.70 g, 121.80 mmol, 2.00 eq.) and AIBN (0.30 g, 1.83 mmol, 0.03 eq.) were added to a solution of methyl 3,4-dimethylbenzoate (10.00 g, 60.90 mmol, 1.00 eq.) in CCH (80.0 mL) and the mixture was stirred at 80° C. for 16 h. The mixture was filtered, and the filtrate was concentrated to give the title compound as yellow oil.

### Step 2: Methyl 2-benzylisoindoline-5-carboxylate

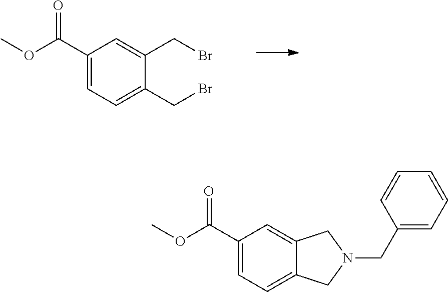 [SMILES: COC(=O)c1ccc(CBr)c(CBr)c1 ; COC(=O)c1ccc2c(c1)CN(Cc1ccccc1)C2]

Phenylmethanamine (2.50 g, 23.30 mmol, 1.00 eq.) in THF (50.0 mL) was added to a stirred mixture of methyl 3,4-bis(bromomethyl)benzoate (7.50 g, 23.30 mmol, 1.00 eq.) and TEA (5.20 g, 51.30 mmol, 2.20 eq.) in THF (100.0 mL). The mixture was stirred at rt for 3 h and then concentrated. The residue was purified by silica gel column chromatography eluting PE/EtOAc (10:1) to give the title compound as yellow solid.

### Step 3: (2-Benzylisoindolin-5-yl) methanol

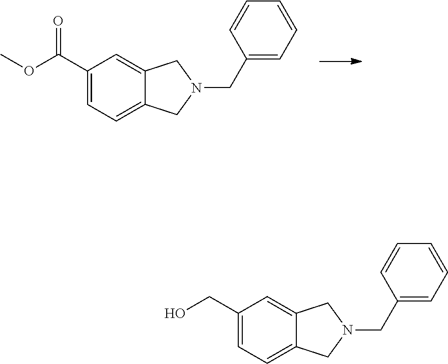 [SMILES: COC(=O)c1ccc2c(c1)CN(Cc1ccccc1)C2 ; OCc1ccc2c(c1)CN(Cc1ccccc1)C2]

A THF (20.0 mL) solution of methyl 2-benzylisoindoline-5-carboxylate (2.00 g, 7.48 mmol, 1.00 eq.) was added dropwise to a stirred suspension of LAH (426 mg, 11.22 mmol, 1.50 eq.) in THF (20.0 mL). The resulting mixture was stirred at rt for 3 h. 1M Na₂SO₄ aq. (2.0 mL) was slowly added and the solids were removed by filtration. The filtrate was concentrated to give the title compound as yellow solid.

### Step 4: Isoindolin-5-ylmethanol

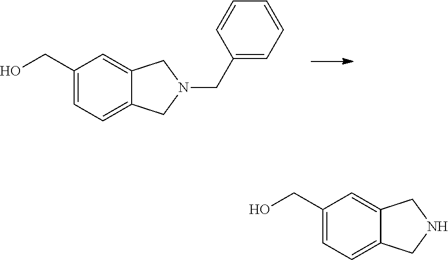 [SMILES: OCc1ccc2c(c1)CN(Cc1ccccc1)C2 ; OCc1ccc2c(c1)CNC2]

Pd/C (10% w/w, 40 mg) was added to a solution of (2-benzylisoindolin-5-yl) methanol (200 mg, 0.84 mmol, 1.00 eq.) in EtOH (10.0 mL) and the mixture was stirred at 50° C. under H₂ (50 psi) overnight. The mixture was filtered and purified by silica gel column chromatography eluting DCM/MeOH (10:1) to give the title compound as a yellow solid.

### Intermediate 11:

### Synthesis of 4-(difluoromethoxy)isoindoline

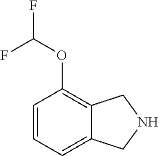 [SMILES: FC(F)Oc1cccc2c1CNC2]

### Step 1: tert-Butyl 4-bromoisoindoline-2-carboxylate

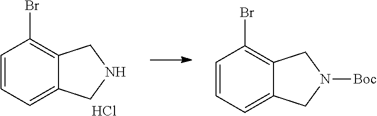 [SMILES: Brc1cccc2c1CNC2 ; Cl ; CC(C)(C)OC(=O)N1Cc2cccc(Br)c2C1]

TEA (10.36 g, 102.34 mmol, 3.00 eq) and Boc₂O (8.93 g, 40.93 mmol, 1.20 eq) were added to a solution of 4-bromoisoindoline hydrochloride (8.00 g, 34.11 mmol, 1.00 eq) in DCM (80.0 mL) and the mixture was stirred at r.t. for 2 h. The mixture was diluted with H₂O and extracted with DCM. The combined organic layers were washed with brine, dried over Na₂SO₄, filtered, and concentrated. The residue was slurred in PE (50.0 mL), and the mixture was filtered and the solid was washed with PE, dried under vacuum to give the title compound as an off-white solid.

### Step 2: tert-Butyl 4-(4,4,5,5-tetramethyl-1,3,2-dioxaborolan-2-yl) isoindoline-2-carboxylate

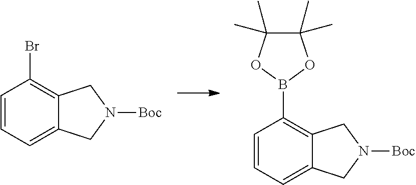 [SMILES: CC(C)(C)OC(=O)N1Cc2cccc(Br)c2C1 ; CC(C)(C)OC(=O)N1Cc2cccc(B3OC(C)(C)C(C)(C)O3)c2C1]

A mixture of tert-butyl 4-bromoisoindoline-2-carboxylate (1.00 g, 3.35 mmol, 1.00 eq), Pd(dppf)Cl₂ (0.25 g, 0.34 mmol, 0.10 eq), 4,4,4′,4′,5,5,5′,5′-octamethyl-2,2′-bi(1,3,2-dioxaborolane) (1.02 g, 4.02 mmol, 1.20 eq) and KOAc (0.66 g, 6.71 mmol, 2.00 eq) in 1,4-dioxane (10.0 mL) was stirred at 100° C. for 8 h under N₂. The mixture was diluted with H₂O and extracted with EtOAc. The combined organic phases were washed with brine, dried over Na₂SO₄, filtered, and concentrated. The residue was dissolved in DCM, passed through a silica pad, and the filtrate was concentrated to give the title compound as a yellow solid.

### Step 3: tert-Butyl 4-hydroxyisoindoline-2-carboxylate

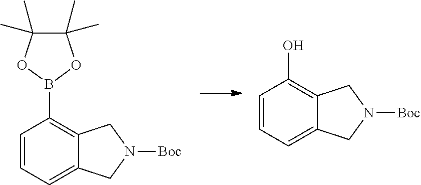 [SMILES: CC(C)(C)OC(=O)N1Cc2cccc(B3OC(C)(C)C(C)(C)O3)c2C1 ; CC(C)(C)OC(=O)N1Cc2cccc(O)c2C1]

NH₄Cl (0.18 g, 3.36 mmol, 0.10 eq), H₂O (10.0 mL) and H₂O₂ (10%, 20.0 mL) were added to a solution of tert-butyl 4-(4,4,5,5-tetramethyl-1,3,2-dioxaborolan-2-yl) isoindoline-2-carboxylate (1.16 g, 3.36 mmol, 1.00 eq) in THF (20.0 mL) and the mixture was stirred at r.t. overnight under N₂. The mixture was extracted with EA, and the combined organic phase was washed with sat. Na₂S₂O₃ aq., water, and brine, dried over Na₂SO₄, filtered, and concentrated. The residue was purified by column chromatography on silica gel (PE:EA=5:1) to give the title compound as off white solid.

### Step 4: tert-Butyl 4-(difluoromethoxy)isoindoline-2-carboxylate

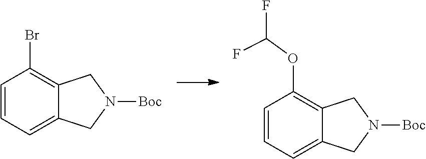 [SMILES: CC(C)(C)OC(=O)N1Cc2cccc(Br)c2C1 ; CC(C)(C)OC(=O)N1Cc2cccc(OC(F)F)c2C1]

NaH (61 mg, 1.53 mmol, 1.20 eq) was added to a solution of tert-butyl 4-hydroxy-isoindoline-2-carboxylate (300 mg, 1.28 mmol, 1.00 eq) in DMF (5.0 mL) and the mixture was stirred at 80° C. for 15 min. CHClF₂ was bubbled into the mixture and the mixture was stirred at 80° C. for 2 h. The mixture was diluted with NH₄Cl aq. and extracted with EA. The combined organic phase was washed with water, brine, dried over Na₂SO₄, filtered, and concentrated. The residue was purified by column chromatography on silica gel (PE:EA=10:1) to give the title compound as a yellow solid.

### Step 5:4-(Difluoromethoxy)isoindoline

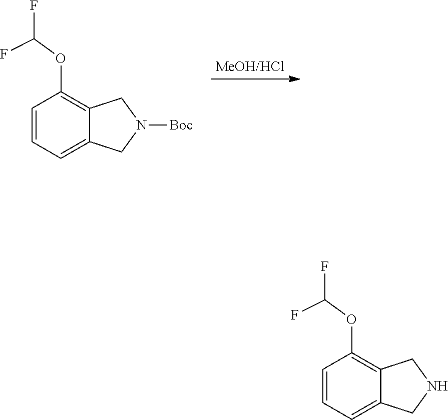 [SMILES: CC(C)(C)OC(=O)N1Cc2cccc(OC(F)F)c2C1 ; FC(F)Oc1cccc2c1CNC2]

MeOH/HCl (2M, 2.0 mL) was added to a solution of tert-butyl 4-(difluoromethoxy)-isoindoline-2-carboxylate (165 mg, 0.58 mmol, 1.00 eq) in MeOH (1.0 mL) and the mixture was stirred at r.t. for 2 h under N₂. The mixture was concentrated to give the title compound as a yellow solid.

### Intermediate 12:

### Synthesis of bicyclo[1.1.1]pentane-1-carboxylic acid

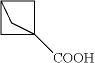

### Step 1: Tricyclo[1.1.1.01,3]pentane

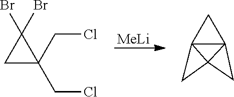 [SMILES: "MeLi" ; ClCC1(CCl)CC1(Br)Br ; C1C23CC24CC134]

MeLi (1.6 M in diethyl ether, 126.3 mL, 202.14 mmol, 2.40 eq.) was added to a mixture of 1,1-dibromo-2,2-bis(chloromethyl)cyclopropane (25.0 g, 84.23 mmol, 1.00 eq.) in diethyl ether (40.0 mL) dropwise at −78° C. This mixture was stirred at −78° C. for 15 min, and then it was warmed to −5° C. and stirred for 2 h. This reaction mixture was warmed to 40° C., and the title compound was obtained as a solution of diethyl ether by vacuum distillation.

### Step 2: Bicyclo[1.1.1]pentan-1-yl(phenyl)sulfane

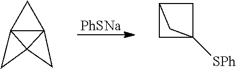

A mixture of sodium benzenethiolate (11.1 g, 84.23 mmol, 1.00 eq.) in H₂O (60.0 mL) was adjusted pH=2~3 with 3M HCl and extracted with MTBE (100.0 mL). A solution of benzenethiol in MTBE was added to a solution of tricyclo[1.1.1.01,3]pentane (5.70 g, 84.23 mmol, 1.00 eq.) in diethyl ether and the mixture was stirred at r.t. under Na for 1 h. The mixture was poured into 1 M NaOH, extracted with MBTE, and the organic layer was washed with brine, dried over Na₂SO₄, filtered, and concentrated. The residue was purified by column chromatography on silica gel (PE:EA=20:1) to give the title compound as a colorless oil.

### Step 3: Bicyclo[1.1.1]pentane-1-carboxylic acid

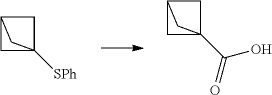

n-BuLi (2.5 M in hexanes, 0.2 mL, 0.57 mmol, 0.10 eq.) was added to a mixture of 4,4′-di-tert-butylbiphenyl (3.00 g, 1.35 mmol, 2.00 eq.) and 1,10-phenantroline (102 mg, 0.57 mmol, 0.10 eq.) in THF (10.0 mL) at −78° C. The mixture was stirred at −78° C. for 30 min, and then at −50° C. for 30 min. A solution of bicyclo[1.1.1]pentan-1-yl(phenyl)sulfane (1.00 g, 5.67 mmol, 1.00 eq.) in THF (5.0 mL) was added to the mixture. Dry CO₂ was bubbled into the solution and the mixture was slowly warmed to r.t. before it was quenched with sat. Na₂CO₃ aq. The mixture was extracted with MTBE and the organic layers were washed with sat. Na₂CO₃. The aqueous layer pH was adjusted to pH=1-2 with 2N HCl aq. and extracted with DCM/MeOH. The combined organic layers were concentrated and the residue was purified by column chromatography on silica gel (DCM/MeOH=50:1) to give the title compound as a brown oil.

### Intermediate 13:

### Synthesis of 1-(difluoromethyl)cyclopropanecarboxylic acid

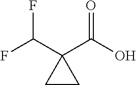

### Step 1: Ethyl 1-formylcyclopropanecarboxylate

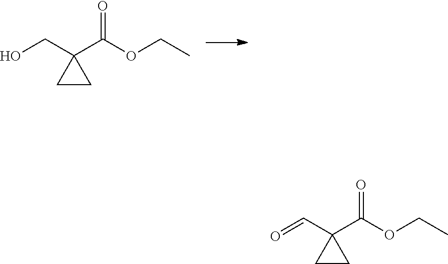

Dess-Martin reagent (110.0 g, 0.26 mol, 1.50 eq.) was added to a stirred mixture of ethyl 1-(hydroxymethyl)cyclopropanecarboxylate (25.00 g, 0.17 mol, 1.00 eq.) in DCM (500.0 mL) and the mixture was stirred at r.t. overnight under N₂. The reaction mixture was concentrated and the residue was purified by column chromatography on silica gel (PE:EA=20:1) to give the title compound as a pale yellow oil.

### Step 2: Ethyl 1-(difluoromethyl)cyclopropanecarboxylate

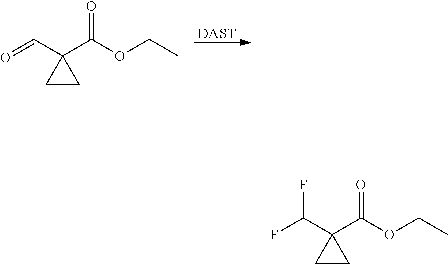

DAST (41.00 g, 0.25 mol, 2.00 eq.) was added to a stirred mixture of ethyl 1-formylcyclopropanecarboxylate (18.00 g, 0.13 mol, 1.00 eq.) in DCM (350.0 mL) at 0° C. and the mixture was stirred at r.t. overnight under N₂. The reaction mixture was diluted with water, and then extracted with EtOAc. The combined organic layers were washed with water, brine, dried over Na₂SO₄, filtered, and concentrated. The residue was purified by column chromatography on silica gel (PE:EA=10:1) to give the title compound as a pale yellow oil.

### Step 3:1-(Difluoromethyl)cyclopropanecarboxylic acid

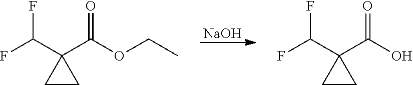

NaOH (7.80 g, 195.12 mmol, 2.00 eq.) was added to a stirred mixture of ethyl 1-(difluoro-methyl)cyclopropanecarboxylate (16.00 g, 97.56 mmol, 1.00 eq.) in MeOH (150.0 mL) and water (75.0 mL) and the mixture was stirred at r.t. overnight. The n mixture was diluted with water, and adjusted pH to 1 by adding 3N HCl aq. The mixture was extracted with EtOAc, and the combined organic layers were washed with water, brine, dried over Na₂SO₄, filtered, and concentrated to give the title compound as a pale yellow oil.

### Intermediate 14:

### Synthesis of 1-(difluoromethyl)cyclobutanecarboxylic acid

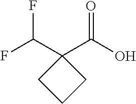

The title compound was prepared with ethyl 1-(hydroxymethyl)cyclobutane-carboxylate replacing ethyl 1-(hydroxymethyl)cyclopropanecarboxylate proceeding analogously as described in Intermediate 13 Step 1.

### Intermediate 15:

### Synthesis of 3-chlorobicyclo[1.1.1]pentane-1-carboxylic acid

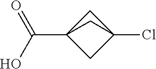

### Step 1: Methyl 3-chlorobicyclo[1.1.1]pentane-1-carboxylate

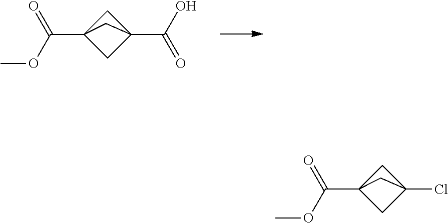

A solution of 3-(methoxycarbonyl)bicyclo[1.1.1]pentane-1-carboxylic acid (500 mg, 2.94 mmol, 1.00 eq.) in Et₂O (12.0 mL) at 0° C. was treated with DMF (21 mg, 0.29 mmol, 0.10 eq.) and oxalyl chloride (821 mg, 6.47 mmol, 2.20 eq.) and the mixture was warmed to r.t. After 70 min., the solvent was removed and the crude product was dissolved in CCL (4.0 mL) in flask A. To a separate flask, B, was added sodium 2-thioxopyridin-1(2H)-olate (533 mg, 3.53 mmol, 1.20 eq.), and CCL (15.0 mL) and the mixture was heated to reflux. The solution of crude acid chloride in CCL of flask A was added dropwise to flask B over 15 min. under irradiation using a halogen work lamp. After 90 min, the mixture was cooled to r.t and diluted with 1M HCl. The mixture was extracted with DCM. The combined organic layers were washed with sat. NaHCO₃, dried over Na₂SO₄, filtered, and concentrated to give the title compound as a pale-yellow oil.

### Step 2: 3-Chlorobicyclo[1.1.1]pentane-1-carboxylic acid

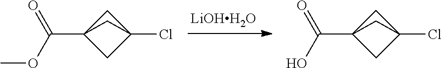

LiOH·H₂O (617 mg, 14.70 mmol, 5.00 eq.) was added to a mixture of methyl 3-chlorobicyclo[1.1.1]pentane-1-carboxylate (472 mg, 2.94 mmol, 1.00 eq.) in THF/H₂O (8.0 mL/2.0 mL) and the mixture was stirred at r.t. overnight. The mixture was concentrated and diluted with H₂O and pH was adjusted to pH=2~3 with 3N HCl aq. The mixture was extracted with DCM, and the combined organic layers were dried over Na₂SO₄, filtered, and concentrated to give the title compound as a pale yellow solid.

### Intermediate 16:

### Synthesis of 4,5-difluoroindoline

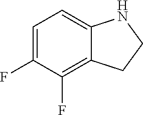 [SMILES: Fc1ccc2c(c1F)CCN2]

NaBH₃CN (247 mg, 3.92 mmol, 3.00 eq.) was added to a mixture of 4,5-difluoro-1H-indole (200 mg, 1.31 mmol, 1.00 eq.) in AcOH (2.0 mL) and the mixture was stirred at rt under N₂ for 1 h. The mixture was extracted with EtOAc and the organic layer was washed with NaHCO₃ aq., brine, dried over Na₂SO₄, filtered and concentrated. The residue was purified by column chromatography on silica gel (PE:EA=10:1) to give the title compound as a pale yellow solid.

### Intermediate 17:

### Synthesis of 6-(difluoromethoxy)indoline

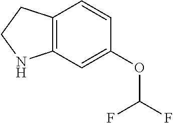 [SMILES: FC(F)Oc1ccc2c(c1)NCC2]

### Step 1: tert-Butyl 6-hydroxy-1H-indole-1-carboxylate

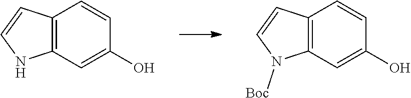 [SMILES: Oc1ccc2cc[nH]c2c1 ; CC(C)(C)OC(=O)n1ccc2ccc(O)cc21]

Boc₂O (4.94 g, 22.56 mmol, 3.00 eq.) and DMAP (100 mg, 0.75 mmol, 0.10 eq.) were added to a stirred solution of 1H-indol-6-ol (1.00 g, 7.52 mmol, 1.00 eq.) in MeCN (10.0 mL) at room temperature. The mixture was stirred at rt for 16 h and K₂CO₃ (7.11 g, 52.66 mmol, 7.00 eq.) which was allowed to stir at rt for 1 h. The reaction mixture was cooled, concentrated and purified by silica gel chromatography (EA:PE=1:5) to give the title compound as a white oil.

### Step 2: tert-Butyl 6-(difluoromethoxy)-1H-indole-1-carboxylate

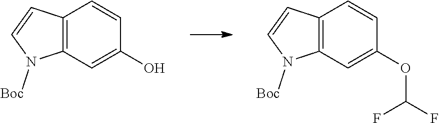 [SMILES: CC(C)(C)OC(=O)n1ccc2ccc(O)cc21 ; CC(C)(C)OC(=O)n1ccc2ccc(OC(F)F)cc21]

NaH (137 mg, 60%, 3.43 mmol, 4.00 eq.) was added to a stirred solution of tert-butyl 6-hydroxy-1H-indole-1-carboxylate (200 mg, 0.86 mmol, 1.00 eq.) in DMF (5.0 mL) at 0° C. CHClF₂ was bubbled into reaction mixture, the mixture was sealed and stirred at 60° C. for 3 h. The mixture was cooled, concentrated, and the residue was purified by silica gel chromatography (EA:PE=1:5) to give the title compound as a white solid.

### Step 3: tert-Butyl 6-(difluoromethoxy)indoline-1-carboxylate

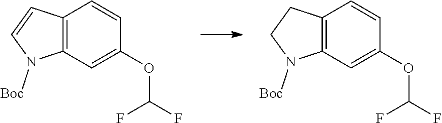 [SMILES: CC(C)(C)OC(=O)n1ccc2ccc(OC(F)F)cc21 ; CC(C)(C)OC(=O)N1CCc2ccc(OC(F)F)cc21]

Pd/C (100 mg) was added to a stirred solution of tert-butyl 6-(difluoromethoxy)-1H-indole-1-carboxylate (25 mg, 0.09 mmol, 1.00 eq.) in EtOAc (10.0 mL), and the resulting mixture was stirred at rt under H₂ atmosphere for 16 h. The reaction mixture was filtered and concentrated to give the title compound as a white oil.

### Step 4:6-(Difluoromethoxy)indoline

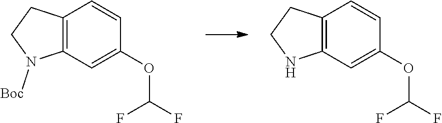 [SMILES: CC(C)(C)OC(=O)N1CCc2ccc(OC(F)F)cc21 ; FC(F)Oc1ccc2c(c1)NCC2]

EtOAc/HCl (2M, 1.0 mL) was added to a stirred solution of tert-butyl 6-(difluoromethoxy)-indoline-1-carboxylate (50 mg, 0.18 mmol, 1.00 eq.) in EtOAc (2.0 mL) under Ne at room temperature and the mixture was stirred for 2 h. The mixture was concentrated to give the title compound as a white solid.

### Intermediate 18:

### Synthesis of 6-ethynylindoline

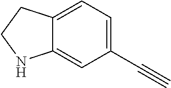

### Step 1:6-((Trimethylsilyl)ethynyl)indoline

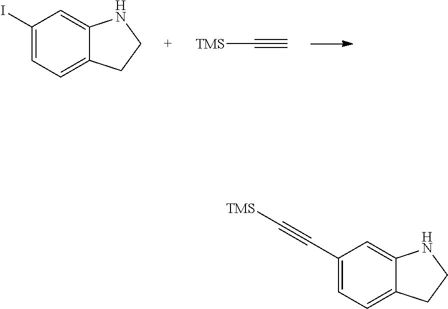

A mixture of 6-iodoindoline (300 mg, 1.22 mmol, 1.00 eq.), ethynyltrimethylsilane (180 mg, 1.84 mmol, 1.50 eq.), CuI (90 mg), TEA (370 mg, 3.66 mmol, 3.00 eq.) and Pd(PPh₃)₂Cl₂ (90 mg). in MeCN (3.0 mL) was stirred at 50° C. overnight. The mixture was concentrated, and the residue was purified by silica gel flash column PE/EtOAc (10:1) to give the title compound as a red oil.

### Step 2: 6-Ethynylindoline

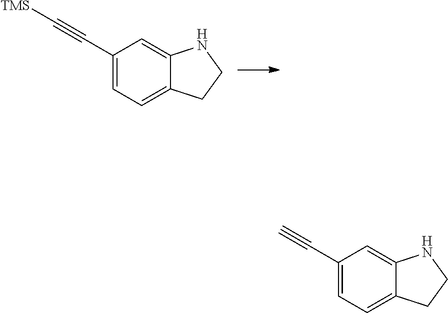

K₂CO₃ (70.6 mg, 0.51 mmol, 1.00 eq.) was added to a mixture of 6-((trimethylsilyl)-ethynyl)indoline (110 mg, 0.51 mmol, 1.00 eq.) in MeOH (2.0 mL) and the mixture was stirred at rt for 3 h. The mixture was concentrated and extracted with DCM. The combined organic layers were concentrated, and the crude product was purified by silica flash column to give the title compound as a yellow oil.

### Intermediate 19:

### Synthesis of 3-(2,2,2-trifluoroethyl)-3,8-diazabicyclo[3.2.1]octane

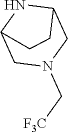 [SMILES: FC(F)(F)CN1CC2CCC(C1)N2]

### Step 1: tert-Butyl 3-(2,2,2-trifluoroethyl)-3,8-diazabicyclo[3.2.1]octane-8-carboxylate

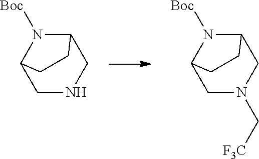 [SMILES: CC(C)(C)OC(=O)N1C2CCC1CNC2 ; CC(C)(C)OC(=O)N1C2CCC1CN(CC(F)(F)F)C2]

2,2,2-Trifluoroethyl trifluoromethanesulfonate (7.87 g, 33.92 mmol, 1.2 eq) was added to a solution of tert-butyl 3,8-diazabicyclo[3.2.1]octane-8-carboxylate (6.00 g, 28.26 mmol, 1.00 eq) in THF (60.0 mL) and TEA (5.72 g, 56.52 mmol, 2.00 eq) and the mixture was stirred at r.t. overnight under N₂. The mixture was concentrated and the residue was purified by flash column chromatography (EA:PE=0 to 100%) to give the title compound as a yellow oil.

### Step 2: 3-(2,2,2-Trifluoroethyl)-3,8-diazabicyclo[3.2.1]octane

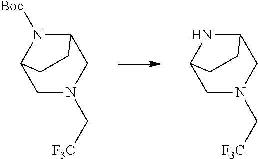 [SMILES: CC(C)(C)OC(=O)N1C2CCC1CN(CC(F)(F)F)C2 ; FC(F)(F)CN1CC2CCC(C1)N2]

EtOAc/HCl (30.0 mL, 2M) was added to a solution of tert-butyl 3-(2,2,2-trifluoroethyl)-3,8-diazabicyclo[3.2.1]octane-8-carboxylate (7.00 g, 23.78 mmol, 1.00 eq) in EtOAc (30.0 mL) and the mixture was stirred at r.t. for 2 h under N₂. The mixture was concentrated to give the title compound as a yellow solid.

### Intermediate 20:

### Synthesis of a Mixture of 6-amino-1-methyl-2-thiaspiro[3.3]heptane 2,2-dioxide and 6-amino-1,3-dimethyl-2-thiaspiro[3.3]heptane 2,2-dioxide

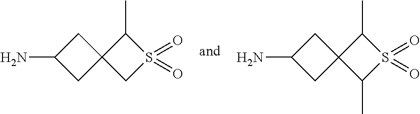

### Step 1:6-(Dibenzylamino)-2-thiaspiro[3.3]heptane 2,2-dioxide

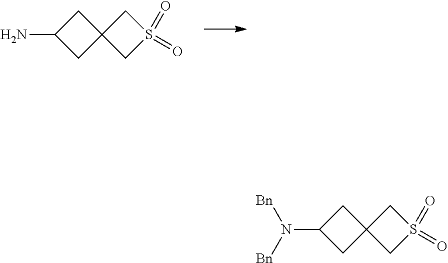 [SMILES: NC1CC2(C1)CS(=O)(=O)C2 ; O=S1(=O)CC2(CC(N(Cc3ccccc3)Cc3ccccc3)C2)C1]

(Bromomethyl)benzene (1.71 g, 10.15 mmol, 2.00 eq.) and K₂CO; (3.50 g, 25.40 mmol, 5.00 eq.) were added to 6-amino-2-thiaspiro[3.3]heptane 2,2-dioxide (1.00 g, 5.08 mmol, 1.00 eq.) in MeCN (10.0 mL) and the mixture was stirred at rt overnight. The mixture was extracted with DCM and the organic layer was concentrated to give the title compound as a white solid.

### Step 2:6-(Dibenzylamino)-1-methyl-2-thiaspiro[3.3]heptane 2,2-dioxide and 6-(dibenzyl-amino)-1,3-dimethyl-2-thiaspiro[3.3]heptane 2,2-dioxide

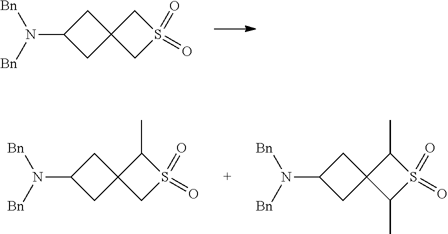 [SMILES: O=S1(=O)CC2(CC(N(Cc3ccccc3)Cc3ccccc3)C2)C1 ; CC1C2(CC(N(Cc3ccccc3)Cc3ccccc3)C2)CS1(=O)=O ; CC1C2(CC(N(Cc3ccccc3)Cc3ccccc3)C2)C(C)S1(=O)=O]

n-BuLi (1.4 mL, 3.53 mmol, 1.50 eq.) was added to a solution of 6-(dibenzylamino)-2-thiaspiro[3.3]heptane 2,2-dioxide (800 mg, 2.35 mmol, 1.00 eq.) in THF (5.0 mL) at −78° C. and the mixture was stirred for 0.5 h. Mel (1.67 g, 11.7 mmol, 5.00 eq.) was added and the mixture was allowed to warm to rt and stirred for 1 h. The mixture was diluted with NH₄Cl aq. and extracted with DCM. The combined organic layers were washed with brine, dried over Na₂SO₄, concentrated, and the residue was purified by silica gel column chromatography eluting with PE/EtOAc (5:1) to give a mixture of the title compounds.

### Step 3: 6-Amino-1-methyl-2-thiaspiro[3.3]heptane 2,2-dioxide and 6-amino-1,3-dimethyl-2-thiaspiro[3.3]heptane 2,2-dioxide

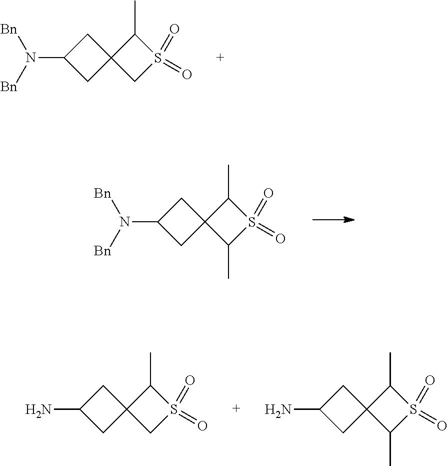 [SMILES: CC1C2(CC(N(Cc3ccccc3)Cc3ccccc3)C2)CS1(=O)=O ; CC1C2(CC(N(Cc3ccccc3)Cc3ccccc3)C2)C(C)S1(=O)=O ; CC1C2(CC(N)C2)CS1(=O)=O ; CC1C2(CC(N)C2)C(C)S1(=O)=O]

A mixture of 6-(dibenzylamino)-1-methyl-2-thiaspiro[3.3]heptane 2,2-dioxide and 6-(dibenzylamino)-1,3-dimethyl-2-thiaspiro[3.3]heptane 2,2-dioxide (800 mg) was dissolved in MeOH (30.0 mL) and Pd/C (80 mg) was added. The mixture was stirred at rt under H₂ atmosphere overnight, then filtrated and concentrated to give a mixture of the title compounds.

### Intermediate 21:

### Synthesis of 3-(((tert-butyldiphenylsilyl)oxy)methyl)bicyclo[1.1.1]pentane-1-carboxylic acid

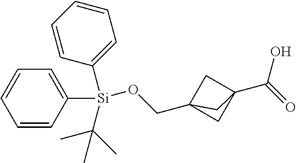 [SMILES: CC(C)(C)[Si](OCC12CC(C(=O)O)(C1)C2)(c1ccccc1)c1ccccc1]

### Step 1: Methyl 3-(hydroxymethyl)bicyclo[1.1.1]pentane-1-carboxylate

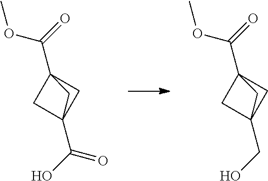

BH₃ in THF (7.80 g, 91.60 mmol, 1.20 eq.) was added to a stirred solution of 3-(methoxycarbonyl)bicyclo[1.1.1]pentane-1-carboxylic acid (10.00 g, 76.33 mmol, 1.00 eq.) in THF (100.0 mL) at 0° C. under N₂. The resulting mixture was stirred at 0° C. 16 h. The reaction mixture was concentrated to give the title compound.

### Step 2: Methyl 3-(((tert-butyldiphenylsilyl)oxy)methyl)bicyclo[1.1.1]pentane-1-carboxylate

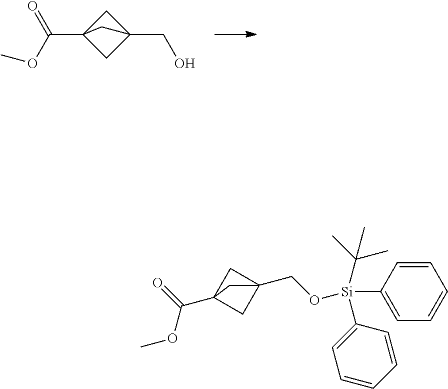 [SMILES: COC(=O)C12CC(CO)(C1)C2 ; COC(=O)C12CC(CO[Si](c3ccccc3)(c3ccccc3)C(C)(C)C)(C1)C2]

TBDPS-Cl (15.78 g, 57.60 mmol, 1.50 eq.) and 1H-imdazole (15.25 g. 76.80 mmol, 2.00 eq.) were added to a solution of methyl 3-(hydroxymethyl)bicyclo[1.1.1]pentane-1-carboxylate (6.00 g, 38.40 mmol, 1.00 eq.) in DMF (10.0 mL) at 0° C. and the resulting mixture was stirred under N₂ at r.t. for 16 h. The reaction mixture was concentrated and the residue was purified by silica gel chromatography (EA:PE=1:5) to give the title compound as a white solid.

### Step 3: 3-(((tert-Butyldiphenylsilyl)oxy)methyl)bicyclo[1.1.1]pentane-1-carboxylic acid

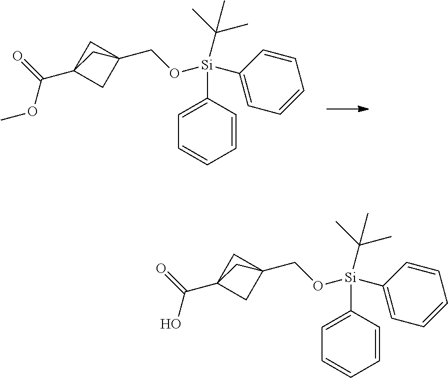 [SMILES: COC(=O)C12CC(CO[Si](c3ccccc3)(c3ccccc3)C(C)(C)C)(C1)C2 ; CC(C)(C)[Si](OCC12CC(C(=O)O)(C1)C2)(c1ccccc1)c1ccccc1]

NaOH (1.98 g, 49.60 mmol, 4.00 eq.) was added to a stirred solution of methyl 3-(((tert-butyldiphenylsilyl)oxy)methyl)bicyclo[1.1.1]pentane-1-carboxylate (4.90 g, 12.40 mmol, 1.00 eq.) in MeOH (50.0 mL) at room temperature. The resulting mixture was stirred at 60° C. for 16 h. The reaction mixture pH was adjusted to pH=6 and extracted with DCM. The combined organic layers were concentrated to give the title compound as a white solid.

### Intermediate 24:

### Synthesis of (1R,5S)-3-methyl-8-azabicyclo[3.2.1]octane-3-carbonitrile

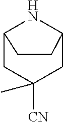

### Step 1: tert-Butyl (1R,5S)-3-cyano-3-methyl-8-azabicyclo[3.2.1]octane-8-carboxylate

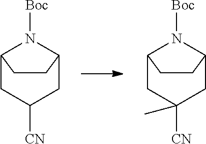 [SMILES: CC(C)(C)OC(=O)N1C2CCC1CC(C#N)C2 ; CC1(C#N)CC2CCC(C1)N2C(=O)OC(C)(C)C]

LDA (1.0 M in THF, 3.2 mL, 3.20 mmol, 3.0 eq.) was added to a solution of tert-butyl (1R,5S)-3-cyano-8-azabicyclo[3.2.1]octane-8-carboxylate (250 mg, 1.06 mmol, 1.00 eq.) in THF (5.0 mL) at −78° C. under N₂ dropwise and the mixture was stirred for 2 h. CHI (226 mg, 1.59 mmol, 1.50 eq.) in THF (1.0 mL) was added. The mixture was stirred at rt overnight before it was diluted with NH₄Cl aq. (5.0 mL). The mixture was extracted with EtOAc, and the combined organic layers were dried over Na₂SO₄, filtered, and concentrated. The residue was purified by column chromatography on silica gel (PE/EA=3:1) to give the title compound as a brown solid.

### Step 2: (1R,5S)-3-methyl-8-azabicyclo[3.2.1]octane-3-carbonitrile

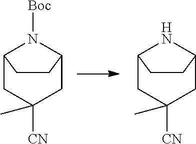 [SMILES: CC1(C#N)CC2CCC(C1)N2C(=O)OC(C)(C)C ; CC1(C#N)CC2CCC(C1)N2]

A mixture of tert-butyl (1R,5S)-3-cyano-3-methyl-8-azabicyclo[3.2.1]octane-8-carboxylate (60 mg, 0.24 mmol, 1.00 eq.) in TFA/DCM (0.5 mL/2.0 mL) was stirred at rt for 2 h. The mixture was concentrated to give the title compound as a yellow oil.

### Intermediate 25:

### Synthesis of 3-(2,2-difluoroethyl)-3,8-diazabicyclo[3.2.1]octane

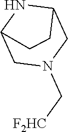 [SMILES: FC(F)CN1CC2CCC(C1)N2]

The title compound was prepared by proceeding analogously as described in Intermediate 19, Step 1, but using 2,2-difluoroethyl trifluoromethanesulfonate instead of 2,2,2-trifluoroethyl trifluoromethanesulfonate.

### Intermediate 26:

### Synthesis of 3-(3,3,3-trifluoropropyl)-3,8-diazabicyclo[3.2.1]octane

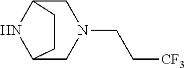 [SMILES: FC(F)(F)CCN1CC2CCC(C1)N2]

### Step 1: tert-Butyl 3-(3,3,3-trifluoropropyl)-3,8-diazabicyclo[3.2.1]octane-8-carboxylate

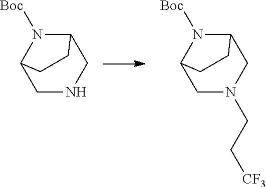 [SMILES: CC(C)(C)OC(=O)N1C2CCC1CNC2 ; CC(C)(C)OC(=O)N1C2CCC1CN(CCC(F)(F)F)C2]

1,1,1-Trifluoro-3-iodopropane (317 mg, 1.41 mmol, 1.50 eq) was added to a mixture of tert-butyl 3,8-diazabicyclo[3.2.1]octane-8-carboxylate (200 mg, 0.94 mmol, 1.00 eq) in DMF (5.0 mL) and K₂CO₃ (260 mg, 1.89 mmol, 2.00 eq) and the resulting mixture was stirred at r.t. overnight and then at 40° C. for 20 h. The mixture was poured in water and extracted with EtOAc. The combined organic layers were washed with water, brine, dried over Na₂SO₄, filtered, and concentrated. The residue was purified by flash column chromatography (EA:PE=0 to 100%) to give the title compound as a yellow oil.

### Step 2:3-(3,3,3-Trifluoropropyl)-3,8-diazabicyclo[3.2.1]octane

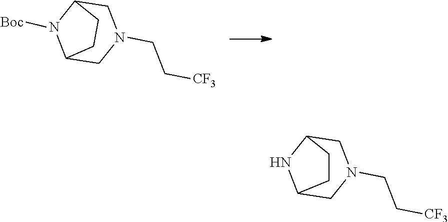 [SMILES: CC(C)(C)OC(=O)N1C2CCC1CN(CCC(F)(F)F)C2 ; FC(F)(F)CCN1CC2CCC(C1)N2]

EtOAc/HCl (2M, 1 mL) was added to a solution of tert-butyl 3-(3,3,3-trifluoropropyl)-3,8-diazabicyclo[3.2.1]octane-8-carboxylate (210 mg, 0.68 mmol, 1.0 eq) in EtOAc (I mL) and the mixture was stirred at r.t. for 2 h. The mixture was concentrated to give the title compound as a yellow solid.

### Intermediate 27:

### Synthesis of 3-benzyl-3,8-diazabicyclo[3.2.1]octane

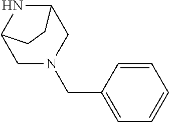 [SMILES: c1ccc(CN2CC3CCC(C2)N3)cc1]

The titled compound was prepared by proceeding analogously as described in Intermediate 26, Step 1, by using (bromomethyl)benzene instead of 1,1,1-trifluoro-3-iodopropane.

### Intermediate 28:

### Synthesis of 3-cyclopropyl-3,8-diazabicyclo[3.2.1]octane

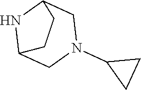 [SMILES: C1CC2CN(C3CC3)CC1N2]

### Step 1: tert-Butyl 3-cyclopropyl-3,8-diazabicyclo[3.2.1]octane-8-carboxylate

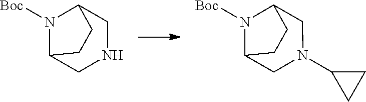 [SMILES: CC(C)(C)OC(=O)N1C2CCC1CNC2 ; CC(C)(C)OC(=O)N1C2CCC1CN(C1CC1)C2]

NaBH₃CN (223 mg, 3.54 mmol, 1.50 eq) was added to a mixture of tert-butyl 3,8-diazabicyclo[3.2.1]octane-8-carboxylate (500 mg, 2.36 mmol, 1.00 eq), (1-ethoxycyclopropoxy)-trimethylsilane (821 mg, 4.71 mmol, 2.00 eq), AcOH (0.5 mL) in MeOH:THF=1:1 (10.0 mL) and the mixture was stirred at r.t. overnight under N₂. The mixture was concentrated and the residue was purified by flash column chromatography (PE:EA=50:1 to 10:1) to give the title compound as a yellow liquid.

### Step 2: 3-Cyclopropyl-3,8-diazabicyclo[3.2.1]octane

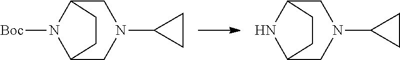 [SMILES: CC(C)(C)OC(=O)N1C2CCC1CN(C1CC1)C2 ; C1CC2CN(C3CC3)CC1N2]

EtOAc/HCl (2M, 3.0 mL) was added to a solution of tert-butyl 3-cyclopropyl-3,8-diazabicyclo[3.2.1]octane-8-carboxylate (485 mg, 1.92 mmol, 1.00 eq) in EtOAc (3.0 mL) and the mixture was stirred at r.t. for 2 h under N₂. The mixture was concentrated to give the title compound as a white solid.

### Intermediate 29:

### Synthesis of 7-azabicyclo[2.2.1]heptane-1-carbonitrile

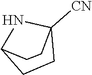

### Step 1: tert-Butyl 7-azabicyclo[2.2.1]heptane-7-carboxylate

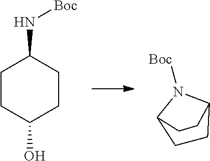 [SMILES: CC(C)(C)OC(=O)N[C@H]1CC[C@H](O)CC1 ; CC(C)(C)OC(=O)N1C2CCC1CC2]

MsCl (9.58 g, 83.61 mmol, 1.50 eq) was added to a solution of tert-butyl ((1r,4r)-4-hydroxycyclohexyl) carbamate (12 g, 55.74 mmol, 1.00 eq) in DCM (200.0 mL) and TEA (8.46 g, 83.61 mmol, 1.50 eq) at 0° C. and the mixture was stirred for 1 h. The mixture was washed with water, brine, and the organic layer was dried over Na₂SO₄, filtered, and concentrated. The resulting product was dissolved in THF (200.0 mL), followed by addition of t-BuOK (6.25 g, 55.74 mmol, 1.00 eq). After 2 h, additional t-BuOK (9.38 g, 83.61 mmol, 1.50 eq) was added and stirring was continued at r.t. overnight under N₂. The mixture was quenched by 1N HCl aq., extracted with EtOAc. The combined organic layers were washed with water, brine, dried over Na₂SO₄, filtered, and concentrated. The residue was purified by flash column chromatography (PE:EA=50:1 to 10:1) to give the title compound as a yellow liquid.

### Step 2: tert-Butyl 1-formyl-7-azabicyclo[2.2.1]heptane-7-carboxylate

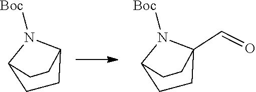 [SMILES: CC(C)(C)OC(=O)N1C2CCC1CC2 ; CC(C)(C)OC(=O)N1C2CCC1(C=O)CC2]

s-BuLi (2.9 mL, 3.80 mmol, 1.50 eq) was added to a solution of tert-butyl 7-azabicyclo-[2.2.1]heptane-7-carboxylate (500 mg, 2.53 mmol, 1.00 eq) and TMEDA (442 mg, 3.80 mmol, 1.50 eq) in Et₂O (5.0 mL) at 0° C. and the mixture was stirred for 1 h under N₂. DMF (370 mg. 5.07 mmol, 2.00 eq) was added and the mixture was stirred at r.t. for 20 h under N₂. The mixture was quenched by sat. NH₄Cl aq. and extracted with EtOAc. The combined organic layers were washed with water, brine, dried over Na₂SO₄, filtered, and concentrated. The residue was purified by flash column chromatography (EA:PE=0 to 100%) to give the title compound as a yellow liquid.

### Step 3: tert-Butyl 1-((hydroxyimino)methyl)-7-azabicyclo[2.2.1]heptane-7-carboxylate

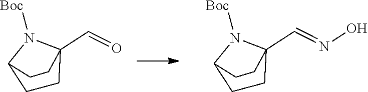 [SMILES: CC(C)(C)OC(=O)N1C2CCC1(C=O)CC2 ; CC(C)(C)OC(=O)N1C2CCC1(/C=N/O)CC2]

NH₂OH·HCl (0.48 g, 6.92 mmol, 1.20 eq) and Na₂CO₃ (0.37 g, 3.46 mmol, 0.60 eq) were added to a solution of tert-butyl 1-formyl-7-azabicyclo[2.2.1]heptane-7-carboxylate (1.30 g, 5.77 mmol, 1.00 eq) in MeOH:H₂O=1:1 (30.0 mL) at r.t. and the mixture was stirred for 3 h. The mixture was poured into water and extracted with EtOAc. The combined organic layers were washed with water, brine, dried over Na₂SO₄, filtered, and concentrated to give the title compound as a yellow oil.

### Step 4: tert-Butyl 1-cyano-7-azabicyclo[2.2.1]heptane-7-carboxylate

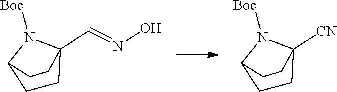 [SMILES: CC(C)(C)OC(=O)N1C2CCC1(/C=N/O)CC2 ; CC(C)(C)OC(=O)N1C2CCC1(C#N)CC2]

Cu(OAc)₂·H₂O (23 mg, 0.12 mmol, 0.02 eq) was added to a solution of tert-butyl 1-((hydroxyimino)methyl)-7-azabicyclo[2.2.1]heptane-7-carboxylate (1.39 g, 5.78 mmol, 1.00 eq) in MeCN (50.0 mL) and the mixture was stirred at 80° C. for 20 h under N₂. The mixture was concentrated and the residue was purified by flash column chromatography (EA:PE=0 to 100%) to give the title compound as a white solid.

### Step 5:7-Azabicyclo[2.2.1]heptane-1-carbonitrile

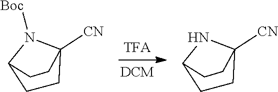 [SMILES: CC(C)(C)OC(=O)N1C2CCC1(C#N)CC2 ; N#CC12CCC(CC1)N2]

TFA (1.0 mL) was added to a solution of tert-butyl 1-cyano-7-azabicyclo[2.2.1]heptane-7-carboxylate (500 mg, 2.25 mmol, 1.00 eq) in DCM (5.0 mL) and the mixture was stirred at r.t. for 2 h. The mixture was concentrated and pH was adjusted to 8 to 9 by adding THF/NH₃. The mixture was concentrated to give the title compound as a yellow solid.

### Intermediate 30:

### Synthesis of 3-(methyl-d3)-3,8-diazabicyclo[3.2.1]octane

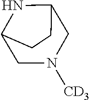 [SMILES: [2H]C([2H])([2H])N1CC2CCC(C1)N2]

The title compound was prepared by proceeding analogously as described in Intermediate 26, Step 1, using iodomethane-d; instead of 1,1,1-trifluoro-3-iodopropane.

### Intermediate 31:

### Synthesis of 3-Phenyl-3,8-diazabicyclo[3.2.1]octane

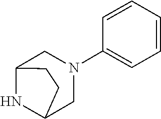 [SMILES: c1ccc(N2CC3CCC(C2)N3)cc1]

### Step 1: tert-Butyl 3-phenyl-3,8-diazabicyclo[3.2.1]octane-8-carboxylate

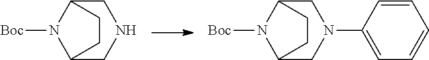 [SMILES: CC(C)(C)OC(=O)N1C2CCC1CNC2 ; CC(C)(C)OC(=O)N1C2CCC1CN(c1ccccc1)C2]

Pd₂(dba)₃ (29 mg, 0.03 mmol, 0.05 eq.), t-BuOK (214 mg, 1.89 mmol, 3.00 eq), BINAP (19.6 mg, 0.0637 mmol, 0.10 eq) were added to a stirred solution of bromobenzene (100 mg, 0.637 mmol, 1.00 eq.) and tert-butyl 3,8-diazabicyclo[3.2.1]octane-8-carboxylate (270 mg, 1.262 mmol, 2.00 eq) in toluene (2.0 mL). The resulting mixture was stirred at 100° C. for 12 h under N₂. The mixture was concentrated and the residue was purified by silica gel column chromatography eluting with PE/EtOAc (10:1) to give the title compound as a white solid.

### Step 2:3-Phenyl-3,8-diazabicyclo[3.2.1]octane

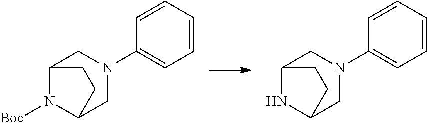 [SMILES: CC(C)(C)OC(=O)N1C2CCC1CN(c1ccccc1)C2 ; c1ccc(N2CC3CCC(C2)N3)cc1]

A mixture of tert-butyl 3-phenyl-3,8-diazabicyclo[3.2.1]octane-8-carboxylate (160 mg, 0.50 mmol, 1.00 eq.) in DCM/TFA=5:1 (2.0 mL) was stirred at room temperature for 2 h. The mixture was concentrated to give the title compound as a white solid.

### Intermediate 32:

### Synthesis of 8-(pyridin-2-yl)-3,8-diazabicyclo[3.2.1]octane

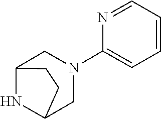 [SMILES: c1ccc(N2CC3CCC(C2)N3)nc1]

The title compound was prepared by proceeding analogously as described in Intermediate 31, Step 1, but using tert-butyl 3,8-diazabicyclo[3.2.1]octane-3-carboxylate instead of tert-butyl 3,8-diazabicyclo[3.2.1]octane-8-carboxylate, and 2-iodopyridine instead of bromobenzene.

### Intermediate 33:

### Synthesis of 3-(pyridin-2-yl)-3,8-diazabicyclo[3.2.1]octane

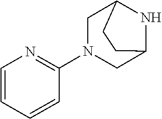 [SMILES: c1ccc(N2CC3CCC(C2)N3)nc1]

The title compound was prepared by proceeding analogously as described in Intermediate 31, Step 1, but using 2-iodopyridine instead of bromobenzene.

### Intermediate 34:

### Synthesis of 4-methyl-4-(methylsulfonyl)cyclohexan-1-amine hydrochloride

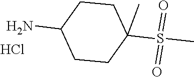

### Step 1: tert-Butyl (4-hydroxy-4-methylcyclohexyl) carbamate

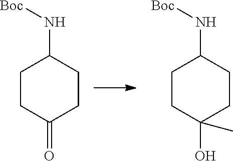 [SMILES: CC(C)(C)OC(=O)NC1CCC(=O)CC1 ; CC1(O)CCC(NC(=O)OC(C)(C)C)CC1]

To a stirred solution of tert-butyl (4-oxocyclohexyl) carbamate (25.00 g, 0.12 mmol, 1.00 eq.) in THF (500.0 mL) was added CH₃MgBr (117 mL, 0.35 mmol, 3.00 eq.) at −78° C. and the mixture was stirred at r.t. for 20 h under N₂. The mixture was poured into sat. NH₄Cl aq., and the mixture was extracted with EtOAc, washed with brine. The organic layer was concentrated and purified by silica gel column chromatography eluting with (EA:PE=0 to 100%) to give the title compound as a pale yellow solid.

### Step 2: 4-((tert-Butoxycarbonyl)amino)-1-methylcyclohexyl methanesulfonate

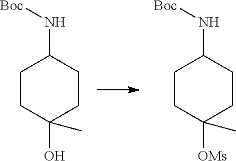 [SMILES: CC1(O)CCC(NC(=O)OC(C)(C)C)CC1 ; CC(C)(C)OC(=O)NC1CCC(C)(OS(C)(=O)=O)CC1]

To a stirred solution of tert-butyl (4-hydroxy-4-methylcyclohexyl) carbamate (8.20 g, 35.81 mmol, 1.00 eq.) in DCM (100.0 mL) was added TEA (15.0 mL, 107.42 mmol, 3.00 eq.), MsCl (3.3 mL, 42.97 mmol, 1.20 eq.) at 0° C. The resulting mixture was stirred at RT for 16 h, and the mixture was washed with brine. The organic layer was dried over Na₂SO₄ and concentrated to give the title compound as a yellow solid.

### Step 3: tert-Butyl (4-methyl-4-(methylthio)cyclohexyl) carbamate

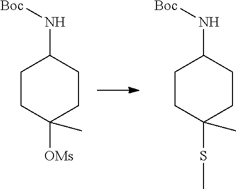 [SMILES: CC(C)(C)OC(=O)NC1CCC(C)(OS(C)(=O)=O)CC1 ; CSC1(C)CCC(NC(=O)OC(C)(C)C)CC1]

To a stirred solution of 4-((tert-butoxycarbonyl)amino)-1-methylcyclohexyl methanesulfonate (11.00 g, 35.83 mmol, 1.00 eq.) in DMF (100.0 mL) was added NaSCH₃ (5.02 g, 71.66 mmol, 2.00 eq.). The reaction mixture was stirred at RT 16 h, and then quenched with H₂O) and then extracted with EtOAc. The organic layer was washed with brine, dried over Na₂SO₄ and concentrated and then purified by reverse phase column chromatography to give the title compound as a white solid.

### Step 4: tert-Butyl (4-methyl-4-(methylsulfonyl)cyclohexyl)carbamate

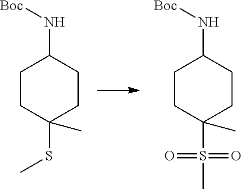 [SMILES: CSC1(C)CCC(NC(=O)OC(C)(C)C)CC1 ; CC(C)(C)OC(=O)NC1CCC(C)(S(C)(=O)=O)CC1]

To a stirred solution of tert-butyl (4-methyl-4-(methylthio)cyclohexyl)-carbamate (500 mg, 1.93 mmol, 1.00 eq.) in MeOH:THF:H₂O=2:2:1 (20.0 mL) was added Oxone (2.37 g, 3.86 mmol, 2.00 eq.). The mixture was stirred at RT 16 h, and then diluted with water and extracted with DCM. The combined organic layer was washed with brine and dried over Na₂SO₄. The organic layer was concentrated to give the title compound as a pale yellow solid.

### Step 5:4-Methyl-4-(methylsulfonyl)cyclohexan-1-amine hydrochloride

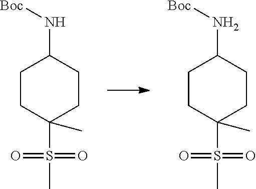 [SMILES: CC(C)(C)OC(=O)NC1CCC(C)(S(C)(=O)=O)CC1 ; CC(C)(C)OC(=O)NC1CCC(C)(S(C)(=O)=O)CC1]

To a stirred solution of tert-butyl (4-methyl-4-(methylsulfonyl)cyclohexyl)-carbamate (380 mg, 1.31 mmol, 1.00 eq.) in EA (2.0 mL) was added EA/HCl (2M, 2.0 mL) and the mixture was stirred for 2 h at r.t. under nitrogen atmosphere. The resulting mixture was concentrated to afford the title compound as a white solid.

### Example 1

### Synthesis of N-(3-(5-(2-(((1R,5S,6r)-3,3-dioxido-3-thiabicyclo-[3.1.0]hexan-6-yl)-amino)pyrimidin-4-yl)-2-(4-methyltetrahydro-2H-pyran-4-yl)thiazol-4-yl)-2-fluorophenyl)-2-oxooxazolidine-3-sulfonamide

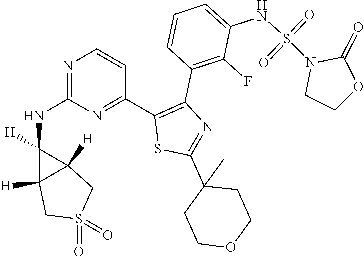 [SMILES: CC1(c2nc(-c3cccc(NS(=O)(=O)N4CCOC4=O)c3F)c(-c3ccnc(N[C@H]4[C@@H]5CS(=O)(=O)C[C@@H]54)n3)s2)CCOCC1]

### Step 1:4-Methyltetrahydro-2H-pyran-4-carboxamide

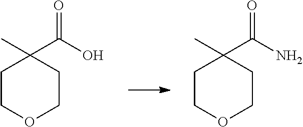

2 Drops of DMF were added to a solution of 4-methyltetrahydro-2H-pyran-4-carboxylic acid (2.50 g, 17.34 mmol, 1.00 eq.) in DCM (20.0 mL), followed by addition of oxalyl chloride (4.40 g, 34.68 mmol, 2.00 eq.) at 0° C. The resulting mixture was stirred at 0° C. under N₂ for 1 h, then concentrated, and dissolved in DCM (30.0 mL). NH₃ (gas) was bubbled through the mixture at 0° C. for 30 min. The mixture was filtered and the organic layer was concentrated to give the title compound.

### Step 2:4-Methyltetrahydro-2H-pyran-4-carbothioamide

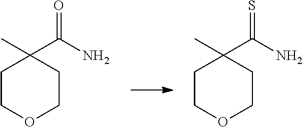

Lawesson's reagent (6.70 g, 16.60 mmol, 1.00 eq.) was added to a stirred solution of 4-methyltetrahydro-2H-pyran-4-carboxamide (2.38 g, 16.60 mmol, 1.00 eq.) in THF (20.0 mL) at room temperature. The resulting mixture was stirred at 50° C. under N₂ for 5 h. The mixture was poured into water and extracted with EtOAc. The combined organic layers were washed with water, brine, dried over Na₂SO₄, filtered, and concentrated. The residue was purified by column chromatography on silica gel (PE:EA=2:1) to give the title compound

### Step 3: N-(3-(5-(2-chloropyrimidin-4-yl)-2-(4-methyltetrahydro-2H-pyran-4-yl)thiazol-4-yl)-2-fluorophenyl)acetamide

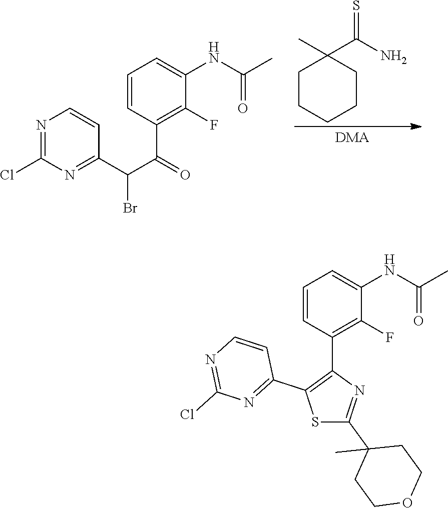

A mixture of N-(3-(2-bromo-2-(2-chloropyrimidin-4-yl)acetyl)-2-fluorophenyl)acetamide (Intermediate 7; 2.43 g, 6.28 mmol, 1.00 eq.) and 4-methyltetrahydro-2H-pyran-4-carbothioamide (1.00 g. 6.28 mmol, 1.00 eq.) in DMA (20.0 mL) was stirred at rt for 30 min and then at 75° C. for 16 h. The mixture was poured into water and extracted with EtOAc. The organic layer was washed with water, brine, dried over Na₂SO₄, filtered, and concentrated. The residue was purified by column chromatography on silica gel (PE:EA=1:1) to give the title compound as a yellow solid.

### Step 4: N-(3-(5-(2-(((1R,5S,6r)-3,3-Dioxido-3-thiabicyclo[3.1.0]hexan-6-yl)amino)pyrimidin-4-yl)-2-(4-methyltetrahydro-2H-pyran-4-yl)thiazol-4-yl)-2-fluorophenyl)acetamide

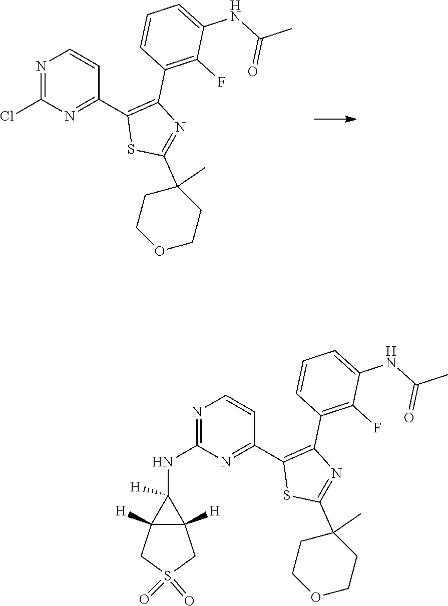 [SMILES: CC(=O)Nc1cccc(-c2nc(C3(C)CCOCC3)sc2-c2ccnc(Cl)n2)c1F ; CC(=O)Nc1cccc(-c2nc(C3(C)CCOCC3)sc2-c2ccnc(N[C@H]3[C@@H]4CS(=O)(=O)C[C@@H]43)n2)c1F]

A mixture of N-(3-(5-(2-chloropyrimidin-4-yl)-2-(4-methyltetrahydro-2H-pyran-4-yl)-thiazol-4-yl)-2-fluorophenyl)acetamide (200 mg, 0.15 mmol, 1.00 eq.), (1R,5S,6r)-6-amino-3-thiabicyclo[3.1.0]hexane 3,3-dioxide (Intermediate 3; 98 mg, 0.67 mmol, 1.50 eq.), RuPhos (30 mg), RuPhos Pd G2 (30 mg) and Cs₂CO; (291 mg, 0.89 mmol, 2.00 eq.) in t-BuOH (6.0 mL) was stirred at 90° C. under N₂ overnight. The mixture was concentrated and the residue was purified by column chromatography on silica gel (DCM:MeOH=50:1) to give the title compound as a yellow solid.

### Step 5: (1R,5S,6r)-6-((4-(4-(3-Amino-2-fluorophenyl)-2-(4-methyltetrahydro-2H-pyran-4-yl)-thiazol-5-yl)pyrimidin-2-yl)amino)-3-thiabicyclo[3.1.0]hexane 3,3-dioxide

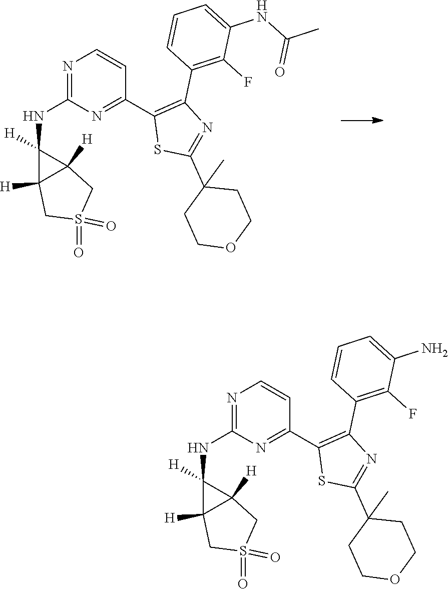 [SMILES: CC(=O)Nc1cccc(-c2nc(C3(C)CCOCC3)sc2-c2ccnc(N[C@H]3[C@@H]4CS(=O)(=O)C[C@@H]43)n2)c1F ; CC1(c2nc(-c3cccc(N)c3F)c(-c3ccnc(N[C@H]4[C@@H]5CS(=O)(=O)C[C@@H]54)n3)s2)CCOCC1]

NaOH (29 mg, 0.72 mmol, 2.00 eq.) was added to a solution of N-(3-(5-(2-(((1R,5S,6r)-3,3-dioxido-3-thiabicyclo[3.1.0]hexan-6-yl)amino)pyrimidin-4-yl)-2-(4-methyltetrahydro-2H-pyran-4-yl)thiazol-4-yl)-2-fluorophenyl)acetamide (200 mg, 0.36 mmol, 1.00 eq.) in EtOH (5.0 mL). This mixture was stirred at 70° C. under N₂ overnight. The mixture was cooled, concentrated and the residue was purified by column chromatography on silica gel (DCM:MeOH=50:1) to give the title compound as a yellow solid.

### Step 6: N-(3-(5-(2-(((1R,5S,6r)-3,3-Dioxido-3-thiabicyclo[3.1.0]hexan-6-yl)amino)pyrimidin-4-yl)-2-(4-methyltetrahydro-2H-pyran-4-yl)thiazol-4-yl)-2-fluorophenyl)-2-oxooxazolidine-3-sulfonamide

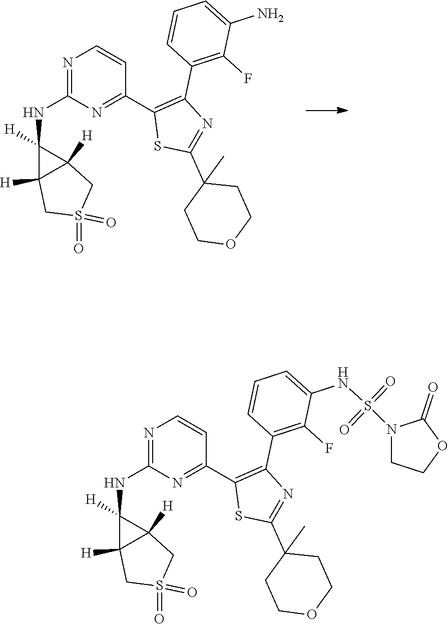 [SMILES: CC1(c2nc(-c3cccc(N)c3F)c(-c3ccnc(N[C@H]4[C@@H]5CS(=O)(=O)C[C@@H]54)n3)s2)CCOCC1 ; CC1(c2nc(-c3cccc(NS(=O)(=O)N4CCOC4=O)c3F)c(-c3ccnc(N[C@H]4[C@@H]5CS(=O)(=O)C[C@@H]54)n3)s2)CCOCC1]

2-Bromoethan-1-ol (153 mg, 1.24 mmol, 2.00 eq.) was added to a mixture of sulfurisocyanatidic chloride (176 mg, 1.24 mmol, 2.00 eq.) in DCM (6.0 mL) at 0° C. under N₂ and it was stirred at 0° C. for 30 min. This reaction mixture was added dropwise to a solution of (1R,5S,6r)-6-((4-(4-(3-amino-2-fluorophenyl)-2-(4-methyltetrahydro-2H-pyran-4-yl)thiazol-5-yl)pyrimidin-2-yl)amino)-3-thiabicyclo[3.1.0]hexane 3,3-dioxide (320 mg, 0.62 mmol, 1.00 eq.) and TEA (314 mg, 3.11 mmol, 5.00 eq.) in DCM (6.0 mL) at 0° C. This mixture was stirred at r.t. for 6 h. Then it was concentrated and purified by column chromatography on silica gel (DCM:MeOH=50:1) to give the title compound as a yellow solid.

### Example 1a

### Synthesis of N-(3-(5-(2-(((1R,5S,6r)-3,3-dioxido-3-thiabicyclo[3.1.0]hexan-6-yl)amino)-pyrimidin-4-yl)-2-(4-methyltetrahydro-2H-pyran-4-yl)thiazol-4-yl)-2-fluorophenyl)-4-methoxyisoindoline-2-sulfonamide

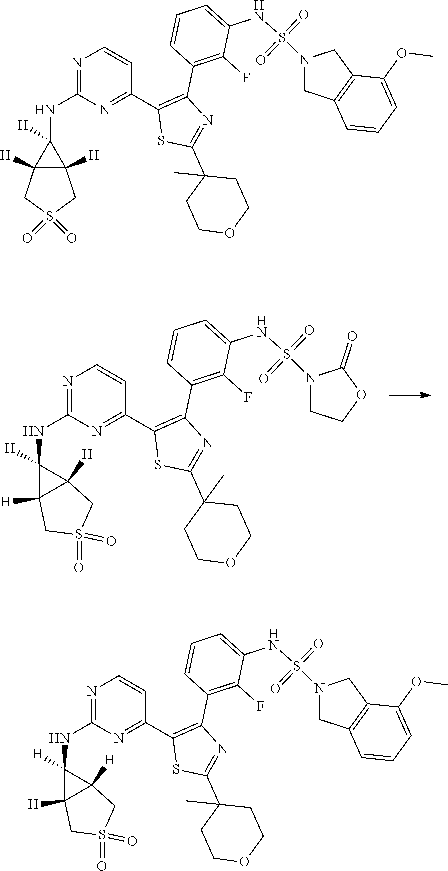 [SMILES: COc1cccc2c1CN(S(=O)(=O)Nc1cccc(-c3nc(C4(C)CCOCC4)sc3-c3ccnc(N[C@H]4[C@@H]5CS(=O)(=O)C[C@@H]54)n3)c1F)C2 ; CC1(c2nc(-c3cccc(NS(=O)(=O)N4CCOC4=O)c3F)c(-c3ccnc(N[C@H]4[C@@H]5CS(=O)(=O)C[C@@H]54)n3)s2)CCOCC1 ; COc1cccc2c1CN(S(=O)(=O)Nc1cccc(-c3nc(C4(C)CCOCC4)sc3-c3ccnc(N[C@H]4[C@@H]5CS(=O)(=O)C[C@@H]54)n3)c1F)C2]

4-Methoxyisoindoline (56 mg, 0.30 mmol, 2.00 eq) was added to a mixture of N-(3-(5-(2-(((1R,5S,6r)-3,3-dioxido-3-thiabicyclo[3.1.0]hexan-6-yl)amino)pyrimidin-4-yl)-2-(4-methyltetrahydro-2H-pyran-4-yl)thiazol-4-yl)-2-fluorophenyl)-2-oxooxazolidine-3-sulfonamide (100 mg, 0.15 mmol, 1.00 eq.) and TEA (76 mg, 0.75 mmol, 5.00 eq.) in CH₃CN (3.0 mL). The mixture was stirred at 100° C. with microwave irradiation for 3 h. The mixture was concentrated and purified by prep-HPLC to give the title compound as a white solid. MS (ES, m/z): [M+1]⁺=727.2.

Proceeding analogously as described in Examples 1 and 1a, the following compounds were prepared.

| | | | LCMS (ES, |
|---|---|---|---|
| | | | m/z): |
| Ex# | Compound | Synthesis | [M + 1]⁺ |
| 2 | 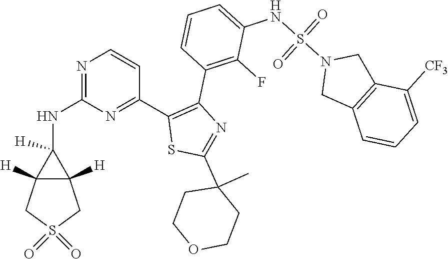 [SMILES: CC1(c2nc(-c3cccc(NS(=O)(=O)N4Cc5cccc(C(F)(F)F)c5C4)c3F)c(-c3ccnc(N[C@H]4[C@@H]5CS(=O)(=O)C[C@@H]54)n3)s2)CCOCC1] | 4-methoxyisoindoline replaced by 4-(trifluoromethyl)isoindoline in Example 1a | 765.2 |
| 3 | 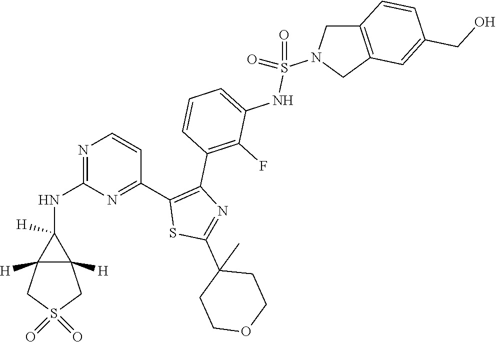 [SMILES: CC1(c2nc(-c3cccc(NS(=O)(=O)N4Cc5ccc(CO)cc5C4)c3F)c(-c3ccnc(N[C@H]4[C@@H]5CS(=O)(=O)C[C@@H]54)n3)s2)CCOCC1] | 4-methoxyisoindoline replaced by Intermediate 10 in Example 1a | 727.3 |
| 4 | 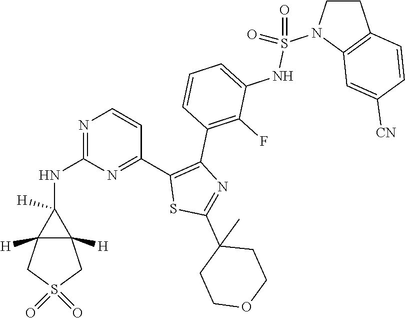 [SMILES: CC1(c2nc(-c3cccc(NS(=O)(=O)N4CCc5ccc(C#N)cc54)c3F)c(-c3ccnc(N[C@H]4[C@@H]5CS(=O)(=O)C[C@@H]54)n3)s2)CCOCC1] | 4-methoxyisoindoline replaced by indoline-6-carbonitrile in Example 1a | 722.3 |
| 5 | 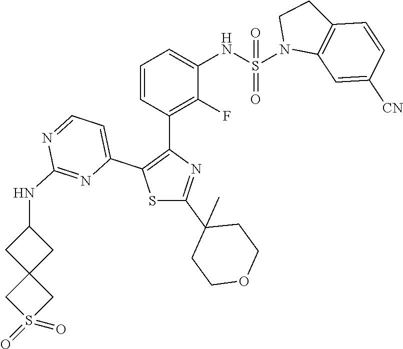 [SMILES: CC1(c2nc(-c3cccc(NS(=O)(=O)N4CCc5ccc(C#N)cc54)c3F)c(-c3ccnc(NC4CC5(C4)CS(=O)(=O)C5)n3)s2)CCOCC1] | 1. Intermediate 3 replaced by Intermediate 6 in Example 1, Step 4. 2. 4-methoxyisoindoline replaced by indoline-6-carbonitrile in Example 1a | 736.2 |
| 6 | 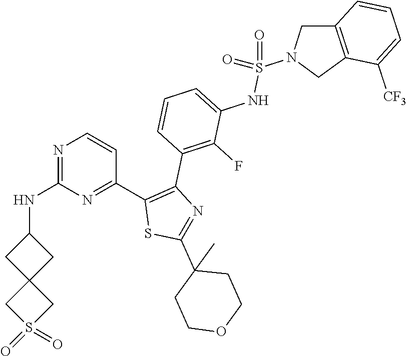 [SMILES: CC1(c2nc(-c3cccc(NS(=O)(=O)N4Cc5cccc(C(F)(F)F)c5C4)c3F)c(-c3ccnc(NC4CC5(C4)CS(=O)(=O)C5)n3)s2)CCOCC1] | 1. Intermediate 3 replaced by Intermediate 6 in Example 1, Step 4. 2. 4-methoxyisoindoline replaced by 4-(trifluoromethyl)- isoindoline in Example 1a | 779.2 |
| 7 | 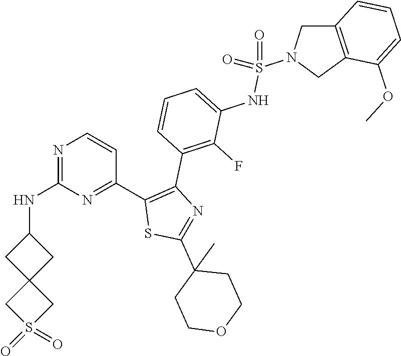 [SMILES: COc1cccc2c1CN(S(=O)(=O)Nc1cccc(-c3nc(C4(C)CCOCC4)sc3-c3ccnc(NC4CC5(C4)CS(=O)(=O)C5)n3)c1F)C2] | Intermediate 3 replaced by Intermediate 6 in Example 1, Step 4. | 741.2 |
| 8 | 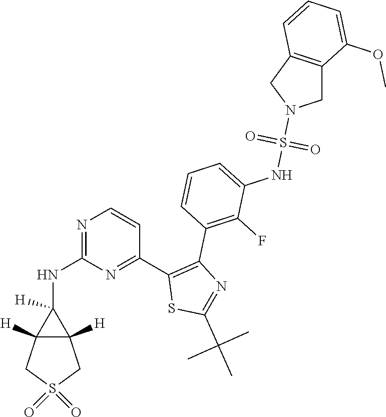 [SMILES: COc1cccc2c1CN(S(=O)(=O)Nc1cccc(-c3nc(C(C)(C)C)sc3-c3ccnc(N[C@H]4[C@@H]5CS(=O)(=O)C[C@@H]54)n3)c1F)C2] | 4-methyltetrahydro-2H-pyran-4- carbothioamide replaced by 2,2- dimethylpropanethioamide in Example 1, Step 3 | 685.2 |
| 9 | 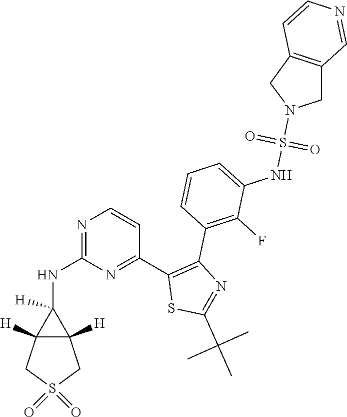 [SMILES: CC(C)(C)c1nc(-c2cccc(NS(=O)(=O)N3Cc4ccncc4C3)c2F)c(-c2ccnc(N[C@H]3[C@@H]4CS(=O)(=O)C[C@@H]43)n2)s1] | 1. 4-methyltetrahydro-2H-pyran- 4-carbothioamide replaced by 2,2-dimethylpropanethioamide in Example 1, Step 3 2. 4-methoxyisoindoline replaced by 2,3-dihydro-1H- pyrrolo[3,4-c]pyridine in Example 1a | 656.2 |

### Example 10

### Synthesis of N-(3-(2-(bicyclo[1.1.1]pentan-1-yl)-5-(2-((2,2-dioxido-2-thiaspiro[3.3]heptan-6-yl)-amino)pyrimidin-4-yl)thiazol-4-yl)-2-fluorophenyl)-6-fluoroindoline-1-sulfonamide

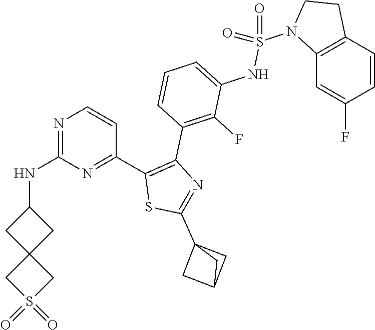 [SMILES: O=S1(=O)CC2(CC(Nc3nccc(-c4sc(C56CC(C5)C6)nc4-c4cccc(NS(=O)(=O)N5CCc6ccc(F)cc65)c4F)n3)C2)C1]

### Step 1: Bicyclo[1.1.1]pentane-1-carboxamide

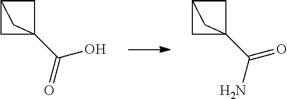 [SMILES: O=C(O)C12CC(C1)C2 ; NC(=O)C12CC(C1)C2]

Oxalyl chloride (294 mg, 2.32 mmol, 1.30 eq.) and DMF (1 drop) was added to a mixture of bicyclo[1.1.1]pentane-1-carboxylic acid (Intermediate 12; 200 mg, 1.78 mmol, 1.00 eq.) in DCM (2.0 mL) at 0° C. The mixture was warmed to r.t. and stirred under N₂ for 2 h. This mixture was added dropwise to a solution of NH₃·H₂O (1.90 g, 53.52 mmol, 30.00 eq.) in THF (3.0 mL) and the resulting mixture was stirred at r.t. overnight. The mixture was diluted with H₂O and extracted with DCM. The combined organic layers were dried over Na₂SO₄, filtered, and concentrated to give the title compound as a yellow solid.

### Step 2: Bicyclo[1.1.1]pentane-1-carbothioamide

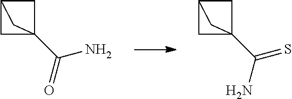 [SMILES: NC(=O)C12CC(C1)C2 ; NC(=S)C12CC(C1)C2]

Lawesson's reagent (655 mg, 1.62 mmol, 1.00 eq.) was added to a mixture of bicyclo[1.1.1]pentane-1-carboxamide (180 mg, 1.62 mmol, 1.00 eq.) in THF (10.0 mL) and the mixture was stirred at 60° C. under N₂ for 3 h. The mixture was concentrated and the residue was purified by column chromatography on silica gel (PE:EA=2:1) to give the title compound as a yellow solid.

### Step 3: N-(3-(2-(Bicyclo[1.1.1]pentan-1-yl)-5-(2-chloropyrimidin-4-yl)thiazol-4-yl)-2-fluorophenyl)acetamide

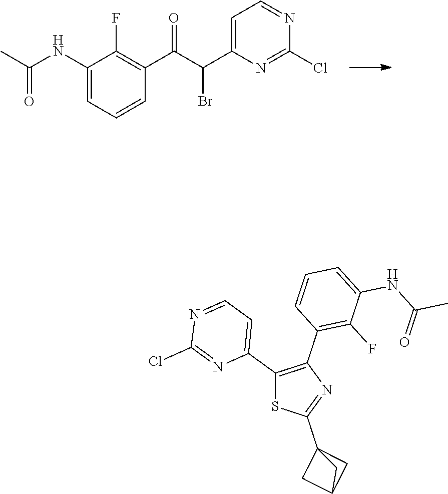 [SMILES: CC(=O)Nc1cccc(C(=O)C(Br)c2ccnc(Cl)n2)c1F ; CC(=O)Nc1cccc(-c2nc(C34CC(C3)C4)sc2-c2ccnc(Cl)n2)c1F]

A mixture of N-(3-(2-bromo-2-(2-chloropyrimidin-4-yl)acetyl)-2-fluorophenyl)acetamide (Intermediate 7; 6.00 g, 15.70 mmol, 1.00 eq) and bicyclo[1.1.1]pentane-1-carbothioamide (2.0 g, 15.70 mmol, 1.00 eq) in DMA (100.0 mL) was stirred at r.t. for 1 h, and then at 65° C. overnight under N₂. The mixture was extracted with EtOAc and the organic layer was washed with water, brine, dried over Na₂SO₄, filtered, and concentrated to give the title compound as a yellow solid.

### Step 4: N-(3-(2-(Bicyclo[1.1.1]pentan-1-yl)-5-(2-((2,2-dioxido-2-thiaspiro[3.3]heptan-6-yl)-amino)pyrimidin-4-yl)thiazol-4-yl)-2-fluorophenyl)acetamide

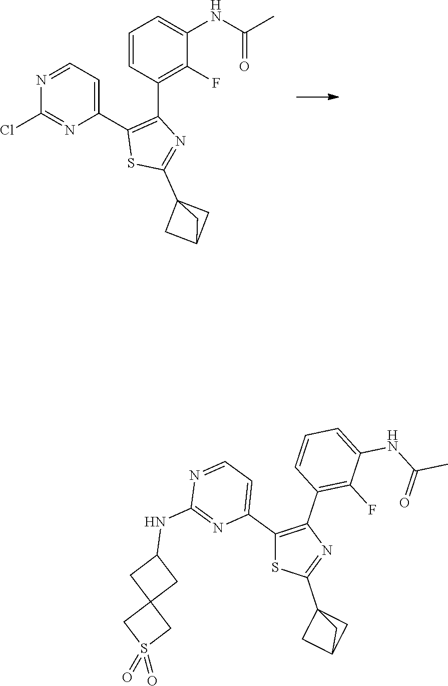 [SMILES: CC(=O)Nc1cccc(-c2nc(C34CC(C3)C4)sc2-c2ccnc(Cl)n2)c1F ; CC(=O)Nc1cccc(-c2nc(C34CC(C3)C4)sc2-c2ccnc(NC3CC4(C3)CS(=O)(=O)C4)n2)c1F]

To a solution of N-(3-(2-(bicyclo[1.1.1]pentan-1-yl)-5-(2-chloropyrimidin-4-yl)thiazol-4-yl)-2-fluorophenyl)acetamide (4.75 g, 11.47 mmol, 1.00 eq) in n-BuOH (100.0 mL) was added 2-thiaspiro[3.3]heptan-6-amine (Intermediate 6; 3.39 g, 17.21 mmol, 1.50 eq), DIEA (8.88 g, 68.84 mmol, 6.00 eq) and the mixture was stirred at 130° C. overnight under N₂. The mixture was concentrated and the residue was purified by column chromatography on silica gel (PE:EA=2:1 to 1:2) to give the title compound as a yellow solid.

### Step 5: 6-((4-(4-(3-Amino-2-fluorophenyl)-2-(bicyclo[1.1.1]pentan-1-yl)thiazol-5-yl)pyrimidin-2-yl)amino)-2-thiaspiro[3.3]heptane 2,2-dioxide

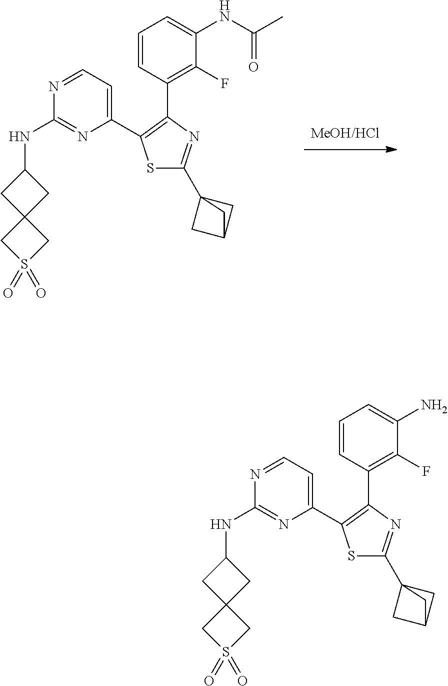 [SMILES: CC(=O)Nc1cccc(-c2nc(C34CC(C3)C4)sc2-c2ccnc(NC3CC4(C3)CS(=O)(=O)C4)n2)c1F ; Nc1cccc(-c2nc(C34CC(C3)C4)sc2-c2ccnc(NC3CC4(C3)CS(=O)(=O)C4)n2)c1F]

To a solution of N-(3-(2-(bicyclo[1.1.1]pentan-1-yl)-5-(2-((2,2-dioxido-2-thiaspiro[3.3]-heptan-6-yl)amino)pyrimidin-4-yl)thiazol-4-yl)-2-fluorophenyl)acetamide (1.5 g, 2.78 mmol) in MeOH (15.0 mL) was added MeOH/HCl (2M, 10.0 mL) and the mixture was stirred at 50° C. for 2 h under N₂. The mixture was concentrated and the pH was adjusted to 9 by addition of 1N NaOH aq. The mixture was extracted with EtOAc. The combined organic layers were washed with water, brine, dried over Na₂SO₄, filtered, concentrated to give the title compound as a yellow solid.

### Step 6: N-(3-(2-(Bicyclo[1.1.1]pentan-1-yl)-5-(2-((2,2-dioxido-2-thiaspiro[3.3]heptan-6-yl)amino)pyrimidin-4-yl)thiazol-4-yl)-2-fluorophenyl)-2-oxooxazolidine-3-sulfonamide

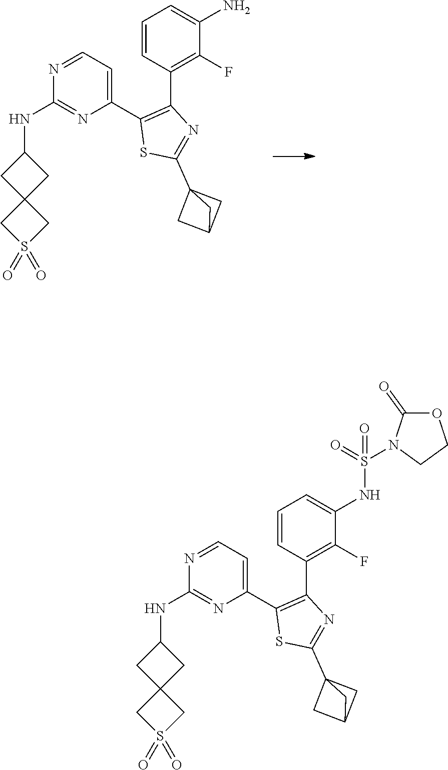 [SMILES: Nc1cccc(-c2nc(C34CC(C3)C4)sc2-c2ccnc(NC3CC4(C3)CS(=O)(=O)C4)n2)c1F ; O=C1OCCN1S(=O)(=O)Nc1cccc(-c2nc(C34CC(C3)C4)sc2-c2ccnc(NC3CC4(C3)CS(=O)(=O)C4)n2)c1F]

To a solution of sulfurisocyanatidic chloride (0.78 g, 5.55 mmol, 2.00 eq) in DCM (15 mL) was added 2-bromoethan-1-ol (0.68 g, 5.55 mmol, 2.00 eq) at 0° C. and stirred at 0° C. 30 min. Then this mixture was added to a solution of 6-((4-(4-(3-amino-2-fluorophenyl)-2-(bicyclo[1.1.1]pentan-1-yl)thiazol-5-yl)pyrimidin-2-yl)amino)-2-thiaspiro[3.3]heptane 2,2-dioxide (1.38 g, 2.78 mmol, 1.0 eq) in DCM (15.0 mL) and TEA (1.40 g, 13.88 mmol, 5.0 eq) at 0° C. and stirred at r.t. for 8 h under N₂. The mixture was washed with water, brine, dried over Na₂SO₄, concentrated. The residue was purified by flash column chromatography (EA:PE=0 to 100%) to give the title compound as a yellow solid.

### Step 7: N-(3-(2-(Bicyclo[1.1.1]pentan-1-yl)-5-(2-((2,2-dioxido-2-thiaspiro[3.3]heptan-6-yl)amino)pyrimidin-4-yl)thiazol-4-yl)-2-fluorophenyl)-6-fluoroindoline-1-sulfonamide

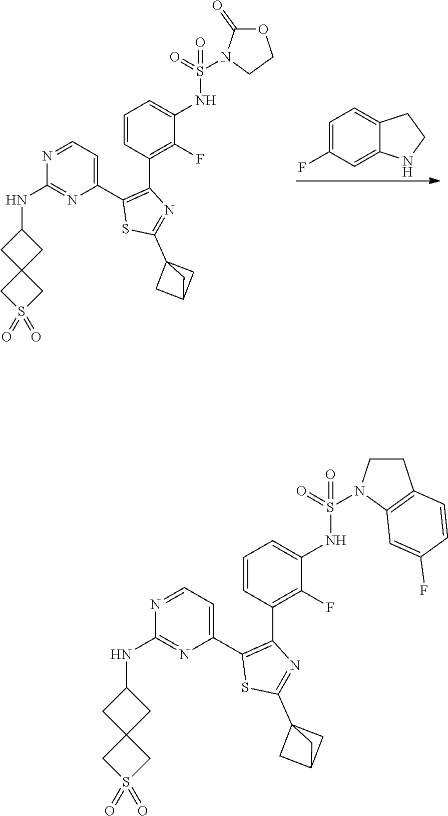 [SMILES: O=C1OCCN1S(=O)(=O)Nc1cccc(-c2nc(C34CC(C3)C4)sc2-c2ccnc(NC3CC4(C3)CS(=O)(=O)C4)n2)c1F ; Fc1ccc2c(c1)NCC2 ; O=S1(=O)CC2(CC(Nc3nccc(-c4sc(C56CC(C5)C6)nc4-c4cccc(NS(=O)(=O)N5CCc6ccc(F)cc65)c4F)n3)C2)C1]

A mixture of N-(3-(2-(bicyclo[1.1.1]pentan-1-yl)-5-(2-((2,2-dioxido-2-thiaspiro[3.3]-heptan-6-yl)amino)pyrimidin-4-yl)thiazol-4-yl)-2-fluorophenyl)-2-oxooxazolidine-3-sulfonamide (100 mg, 0.15 mmol, 1.00 eq), 6-fluoroindoline (32 mg, 0.23 mmol, 1.50 eq), TEA (94 mg, 0.93 mmol, 6.00 eq) in MeCN (2.0 mL) was stirred at 100° C. for 3 h under microwave irradiation. The mixture was concentrated and the residue was purified by flash column chromatography (EA:PE=0 to 100%) to give the title compound as a pale yellow solid. MS (ES, m/z): [M+1]⁺=697.2.

Proceeding analogously as described in Example 10, the following compounds were prepared.

| | | | LCMS |
|---|---|---|---|
| | | | (ES, m/z); |
| Ex# | Compound | Synthesis | [M + 1]⁺ |
| 11 | 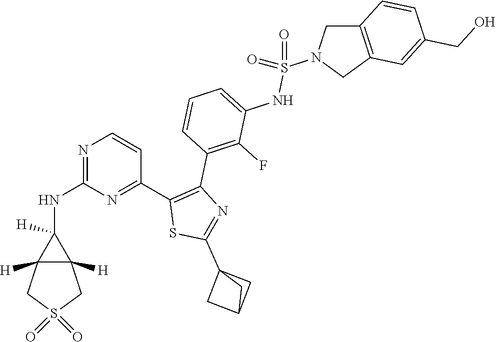 [SMILES: O=S1(=O)C[C@@H]2[C@H](C1)[C@H]2Nc1nccc(-c2sc(C34CC(C3)C4)nc2-c2cccc(NS(=O)(=O)N3Cc4ccc(CO)cc4C3)c2F)n1] | 1. Intermediate 6 replaced by Intermediate 3 in Step 4. 2. 6-fluoroindoline replaced by Intermediate 10 in Step 7. | 695.2 |
| 12 | 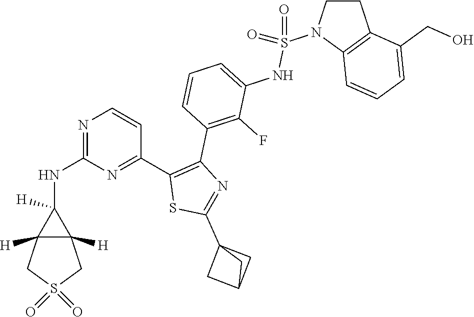 [SMILES: O=S1(=O)C[C@@H]2[C@H](C1)[C@H]2Nc1nccc(-c2sc(C34CC(C3)C4)nc2-c2cccc(NS(=O)(=O)N3CCc4c(CO)cccc43)c2F)n1] | 1. Intermediate 6 replaced by Intermediate 3 in Step 4. 2. 6-fluoroindoline replaced by indolin-4-ylmethanol in Step 7. | 695.2 |
| 13 | 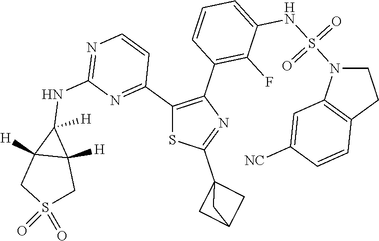 [SMILES: N#Cc1ccc2c(c1)N(S(=O)(=O)Nc1cccc(-c3nc(C45CC(C4)C5)sc3-c3ccnc(N[C@H]4[C@@H]5CS(=O)(=O)C[C@@H]54)n3)c1F)CC2] | 1. Intermediate 6 replaced by Intermediate 3 in Step 4. 2. 6-fluoroindoline replaced by indoline-6-carbonitrile in Step 7. | 690.2 |
| 14 | 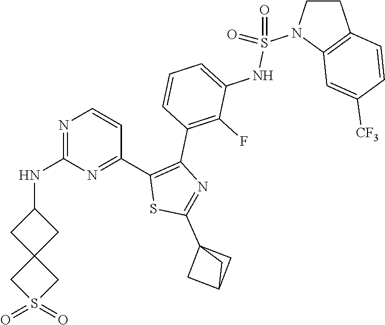 [SMILES: O=S1(=O)CC2(CC(Nc3nccc(-c4sc(C56CC(C5)C6)nc4-c4cccc(NS(=O)(=O)N5CCc6ccc(C(F)(F)F)cc65)c4F)n3)C2)C1] | 6-fluoroindoline replaced by 6- (trifluoromethyl)indoline in Step 7. | 747.2 |
| 15 | 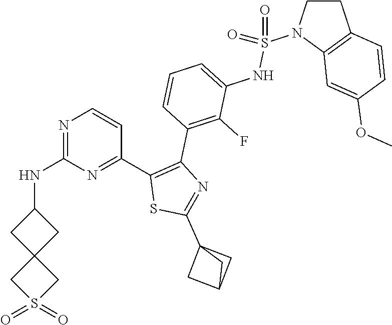 [SMILES: COc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | 6-fluoroindoline replaced by 6- methoxyindoline in Step 7. | 709.2 |
| 16 | 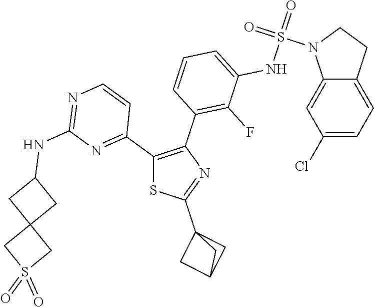 [SMILES: O=S1(=O)CC2(CC(Nc3nccc(-c4sc(C56CC(C5)C6)nc4-c4cccc(NS(=O)(=O)N5CCc6ccc(Cl)cc65)c4F)n3)C2)C1] | 6-fluoroindoline replaced by 6- chloroindoline in Step 7. | 713.2 |
| 17 | 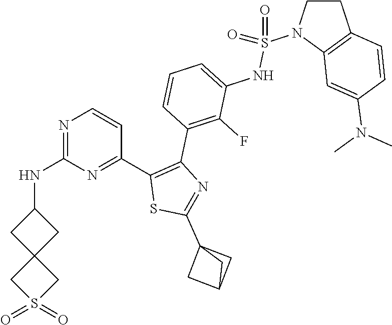 [SMILES: CN(C)c1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | 6-fluoroindoline replaced by N,N-dimethylindolin-6- amine in Step 7. | 722.3 |
| 18 | 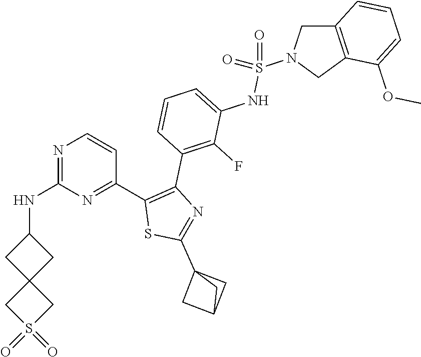 [SMILES: COc1cccc2c1CN(S(=O)(=O)Nc1cccc(-c3nc(C45CC(C4)C5)sc3-c3ccnc(NC4CC5(C4)CS(=O)(=O)C5)n3)c1F)C2] | 6-fluoroindoline replaced by 4- methoxyisoindoline in Step 7. | 709.2 |
| 19 | 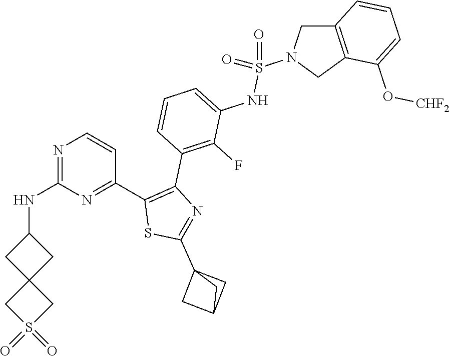 [SMILES: O=S1(=O)CC2(CC(Nc3nccc(-c4sc(C56CC(C5)C6)nc4-c4cccc(NS(=O)(=O)N5Cc6cccc(OC(F)F)c6C5)c4F)n3)C2)C1] | 6-fluoroindoline replaced by Intermediate 11 in Step 7. | 745.2 |
| 20 | 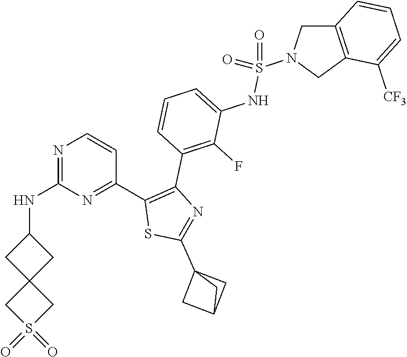 [SMILES: O=S1(=O)CC2(CC(Nc3nccc(-c4sc(C56CC(C5)C6)nc4-c4cccc(NS(=O)(=O)N5Cc6cccc(C(F)(F)F)c6C5)c4F)n3)C2)C1] | 6-fluoroindoline replaced by 4- (trifluoromethyl)isoindoline in Step 7. | 747.2 |
| 21 | 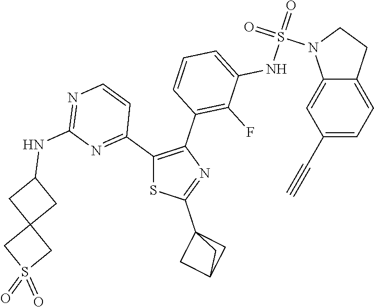 [SMILES: C#Cc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | 6-fluoroindoline replaced by Intermediate 18 in Step 7. | 703.3 |
| 22 | 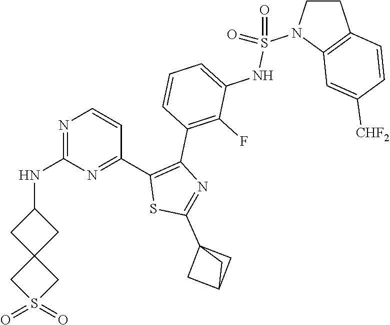 [SMILES: O=S1(=O)CC2(CC(Nc3nccc(-c4sc(C56CC(C5)C6)nc4-c4cccc(NS(=O)(=O)N5CCc6ccc(C(F)F)cc65)c4F)n3)C2)C1] | 6-fluoroindoline replaced by Intermediate 9 in Step 7. | 729.2 |
| 23 | 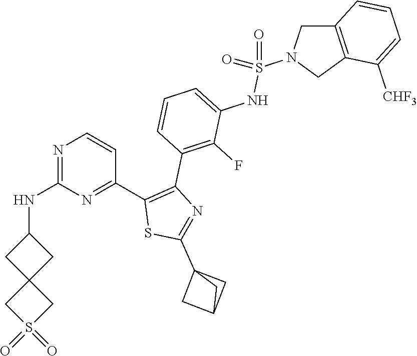 | 6-fluoroindoline replaced by Intermediate 8 in Step 7. | 729.2 |
| 25 | 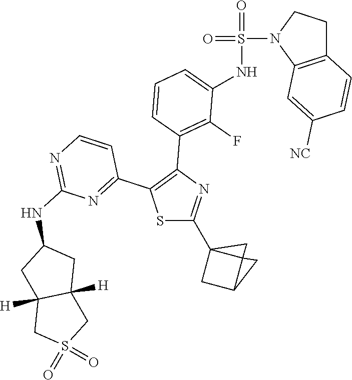 [SMILES: N#Cc1ccc2c(c1)N(S(=O)(=O)Nc1cccc(-c3nc(C45CC6CC64C5)sc3-c3ccnc(N[C@H]4C[C@@H]5CS(=O)(=O)C[C@@H]5C4)n3)c1F)CC2] | 1. Intermediate 6 replaced by Intermediate 1 in Step 4. 2. 6-fluoroindoline replaced by indoline-6-carbonitrile in Step 7. | 718.1 |
| 26 | 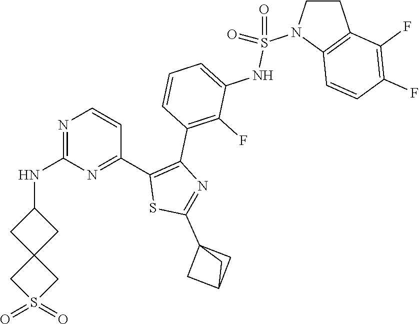 [SMILES: O=S1(=O)CC2(CC(Nc3nccc(-c4sc(C56CC(C5)C6)nc4-c4cccc(NS(=O)(=O)N5CCc6c5ccc(F)c6F)c4F)n3)C2)C1] | 6-fluoroindoline replaced by Intermediate 16 in Step 7 | 715.3 |
| 27 | 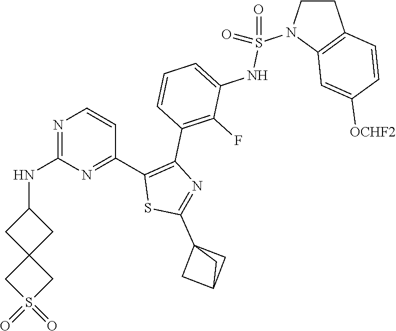 [SMILES: O=S1(=O)CC2(CC(Nc3nccc(-c4sc(C56CC(C5)C6)nc4-c4cccc(NS(=O)(=O)N5CCc6ccc(OC(F)F)cc65)c4F)n3)C2)C1] | 6-fluoroindoline replaced by Intermediate 17 in Step 7. | 745.3 |
| 28 | 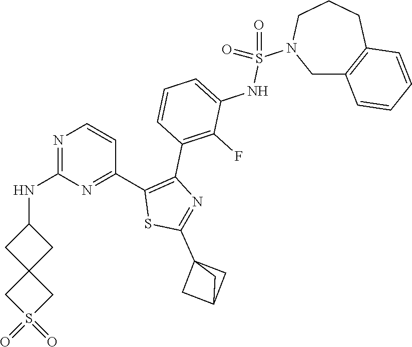 [SMILES: O=S1(=O)CC2(CC(Nc3nccc(-c4sc(C56CC(C5)C6)nc4-c4cccc(NS(=O)(=O)N5CCCc6ccccc6C5)c4F)n3)C2)C1] | 6-fluoroindoline replaced by 2,3,4,5-tetrahydro-1H-benzo [c]azepine in Step 7. | 707.2 |
| 29 | 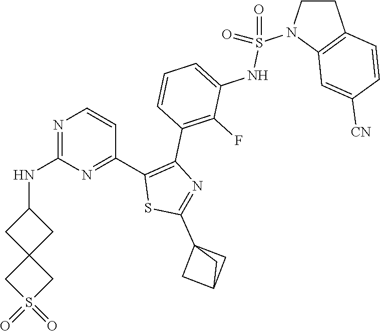 [SMILES: N#Cc1ccc2c(c1)N(S(=O)(=O)Nc1cccc(-c3nc(C45CC(C4)C5)sc3-c3ccnc(NC4CC5(C4)CS(=O)(=O)C5)n3)c1F)CC2] | 6-fluoroindoline replaced by indoline-6-carbonitrile in Step 7. | 704.3 |
| 30 | 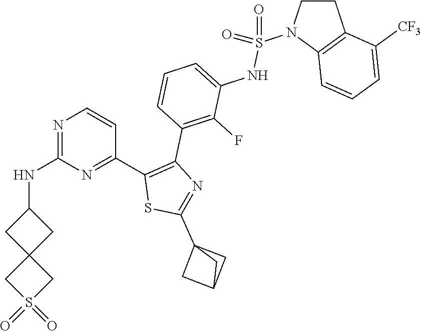 [SMILES: O=S1(=O)CC2(CC(Nc3nccc(-c4sc(C56CC(C5)C6)nc4-c4cccc(NS(=O)(=O)N5CCc6c5cccc6C(F)(F)F)c4F)n3)C2)C1] | 6-fluoroindoline replaced by 4- (trifluoromethyl)indoline in Step 7. | 747.2 |
| 31 | 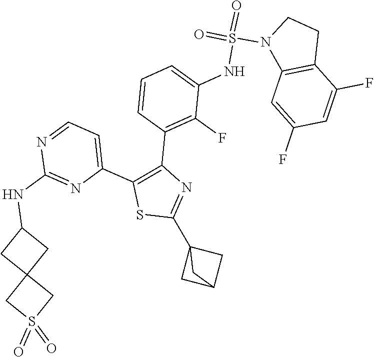 [SMILES: O=S1(=O)CC2(CC(Nc3nccc(-c4sc(C56CC(C5)C6)nc4-c4cccc(NS(=O)(=O)N5CCc6c(F)cc(F)cc65)c4F)n3)C2)C1] | 6-fluoroindoline replaced by 4,6- difluoroindoline in Step 7. | 715.2 |
| 32 | 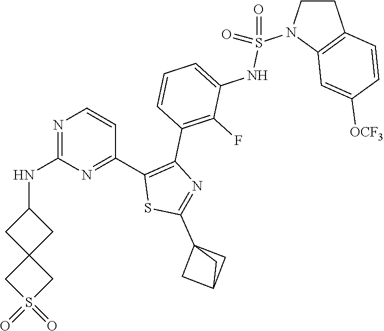 [SMILES: O=S1(=O)CC2(CC(Nc3nccc(-c4sc(C56CC(C5)C6)nc4-c4cccc(NS(=O)(=O)N5CCc6ccc(OC(F)(F)F)cc65)c4F)n3)C2)C1] | 6-fluoroindoline replaced by 6- (trifluoromethoxy)indoline in Step 7. | 763.2 |
| 33 | 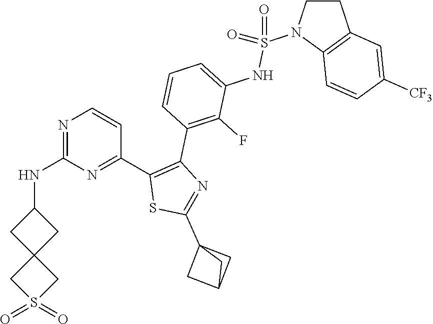 [SMILES: O=S1(=O)CC2(CC(Nc3nccc(-c4sc(C56CC(C5)C6)nc4-c4cccc(NS(=O)(=O)N5CCc6cc(C(F)(F)F)ccc65)c4F)n3)C2)C1] | 6-fluoroindoline replaced by 5- (trifluoromethyl)indoline in Step 7. | 747.2 |
| 34 | 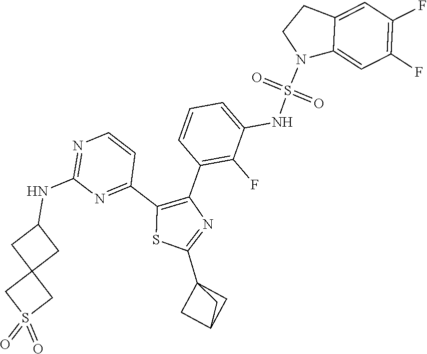 [SMILES: O=S1(=O)CC2(CC(Nc3nccc(-c4sc(C56CC(C5)C6)nc4-c4cccc(NS(=O)(=O)N5CCc6cc(F)c(F)cc65)c4F)n3)C2)C1] | 6-fluoroindoline replaced by 5,6- difluoroindoline in Step 7. | 715.2 |
| 35 | 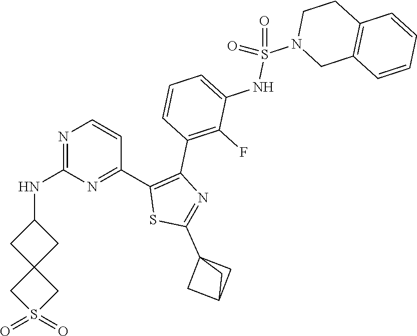 [SMILES: O=S1(=O)CC2(CC(Nc3nccc(-c4sc(C56CC(C5)C6)nc4-c4cccc(NS(=O)(=O)N5CCc6ccccc6C5)c4F)n3)C2)C1] | 6-fluoroindoline replaced by 1,2,3,4-tetrahydroisoquinoline in Step 7. | 693.3 |
| 36 | 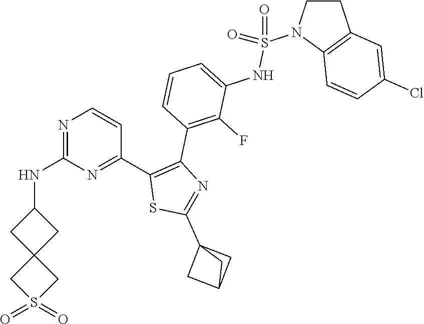 [SMILES: O=S1(=O)CC2(CC(Nc3nccc(-c4sc(C56CC(C5)C6)nc4-c4cccc(NS(=O)(=O)N5CCc6cc(Cl)ccc65)c4F)n3)C2)C1] | 6-fluoroindoline replaced by 5- chloroindoline in Step 7. | 713.2 |
| 37 | 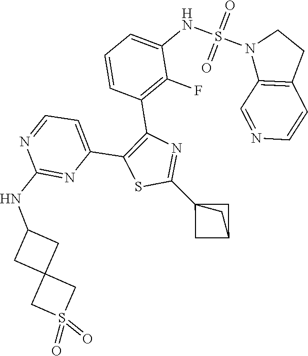 [SMILES: O=S1(=O)CC2(CC(Nc3nccc(-c4sc(C56CC(C5)C6)nc4-c4cccc(NS(=O)(=O)N5CCc6ccncc65)c4F)n3)C2)C1] | 6-fluoroindoline replaced by 2,3-dihydro-1H-pyrrolo[2,3-c] pyridine in Step 7. | 680.2 |
| 38 | 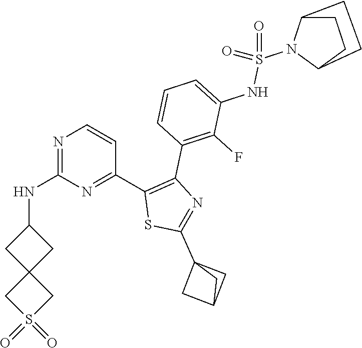 [SMILES: O=S1(=O)CC2(CC(Nc3nccc(-c4sc(C56CC(C5)C6)nc4-c4cccc(NS(=O)(=O)N5C6CCC5CC6)c4F)n3)C2)C1] | 6-fluoroindoline replaced by 7- azabicyclo[2.2.1]heptane in Step 7. | 657.2 |
| 39 | 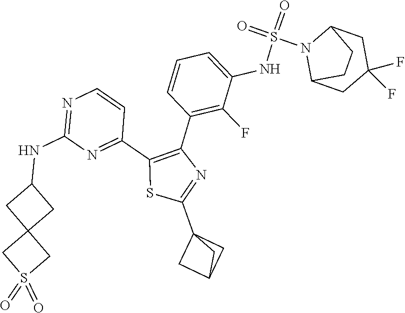 [SMILES: O=S1(=O)CC2(CC(Nc3nccc(-c4sc(C56CC(C5)C6)nc4-c4cccc(NS(=O)(=O)N5C6CCC5CC(F)(F)C6)c4F)n3)C2)C1] | 6-fluoroindoline replaced by 3,3-difluoro-8-azabicyclo [3.2.1]octane in Step 7. | 707.2 |
| 40 | 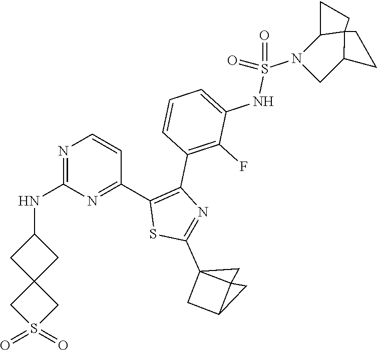 [SMILES: O=S1(=O)CC2(CC(Nc3nccc(-c4sc(C56CC7CC75C6)nc4-c4cccc(NS(=O)(=O)N5CC6CCC67CCC57)c4F)n3)C2)C1] | 6-fluoroindoline replaced by (1s,4s)-2-azabicyclo[2.2.2] octane in Step 7. | 671.2 |
| 41 | 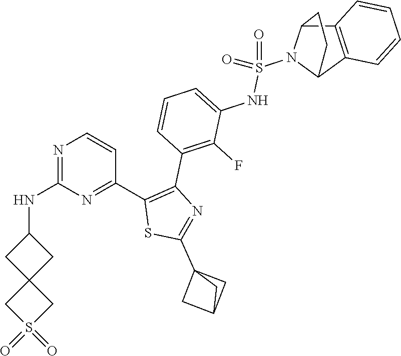 [SMILES: O=S1(=O)CC2(CC(Nc3nccc(-c4sc(C56CC(C5)C6)nc4-c4cccc(NS(=O)(=O)N5C6CCC5c5ccccc56)c4F)n3)C2)C1] | 6-fluoroindoline replaced by 1,2,3,4-tetrahydro-1,4- epiminonaphthalene in Step 7. | 705.2 |
| 42 | 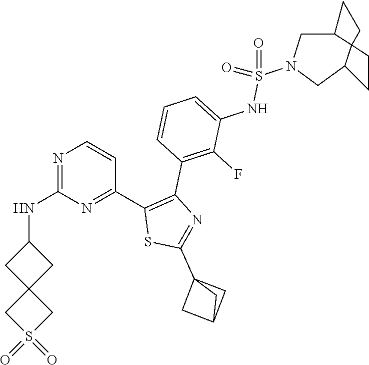 [SMILES: O=S1(=O)CC2(CC(Nc3nccc(-c4sc(C56CC(C5)C6)nc4-c4cccc(NS(=O)(=O)N5CC6CCC67CCC7C5)c4F)n3)C2)C1] | 6-fluoroindoline replaced by (1s,5s)-3-azabicyclo[3.2.2] nonane in Step 7. | 685.2 |
| 43 | 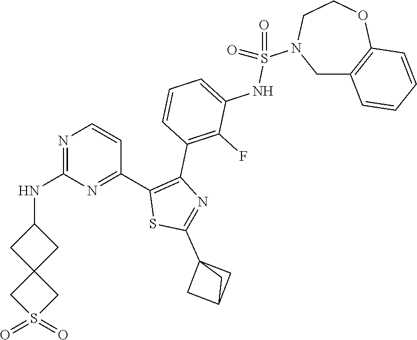 [SMILES: O=S1(=O)CC2(CC(Nc3nccc(-c4sc(C56CC(C5)C6)nc4-c4cccc(NS(=O)(=O)N5CCOc6ccccc6C5)c4F)n3)C2)C1] | 6-fluoroindoline replaced by 2,3,4,5-tetrahydrobenzo[f][1,4] oxazepine in Step 7. | 709.3 |
| 44 | 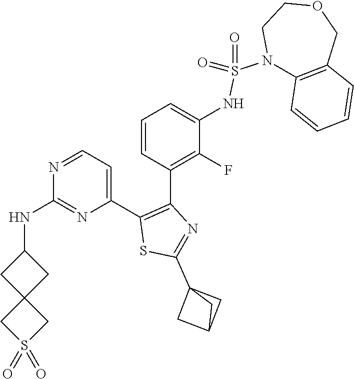 [SMILES: O=S1(=O)CC2(CC(Nc3nccc(-c4sc(C56CC(C5)C6)nc4-c4cccc(NS(=O)(=O)N5CCOCc6ccccc65)c4F)n3)C2)C1] | 6-fluoroindoline replaced by 1,2,3,5-tetrahydrobenzo[e][1,4] oxazepine in Step 7. | 709.2 |
| 45 | 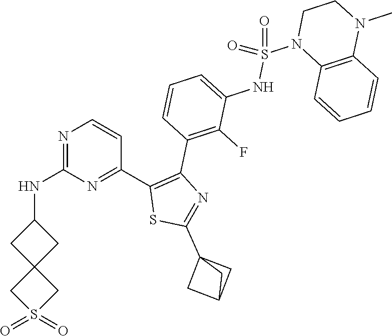 [SMILES: CN1CCN(S(=O)(=O)Nc2cccc(-c3nc(C45CC(C4)C5)sc3-c3ccnc(NC4CC5(C4)CS(=O)(=O)C5)n3)c2F)c2ccccc21] | 6-fluoroindoline replaced by 1-methyl-1,2,3,4-tetrahydro- quinoxaline in Step 7. | 708.2 |
| 46 | 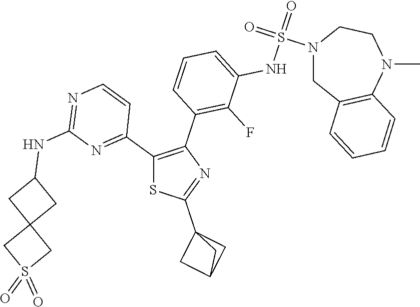 [SMILES: CN1CCN(S(=O)(=O)Nc2cccc(-c3nc(C45CC(C4)C5)sc3-c3ccnc(NC4CC5(C4)CS(=O)(=O)C5)n3)c2F)Cc2ccccc21] | 6-fluoroindoline replaced by 1-methyl-2,3,4,5-tetrahydro-1H- benzo[e][1,4]diazepine in Step 7. | 722.4 |
| 47 | 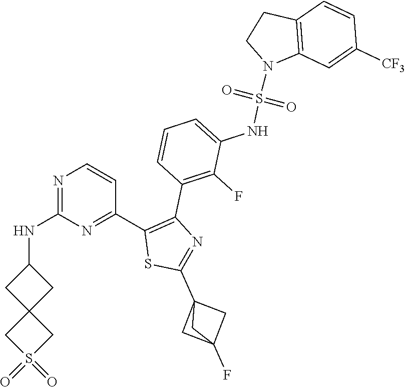 [SMILES: O=S1(=O)CC2(CC(Nc3nccc(-c4sc(C56CC(F)(C5)C6)nc4-c4cccc(NS(=O)(=O)N5CCc6ccc(C(F)(F)F)cc65)c4F)n3)C2)C1] | 1. bicyclo[1.1.1]pentane-1- carboxylic acid was replaced by 3-fluorobicyclo[1.1.1] pentane-1-carboxylic acid in Step 1 2. 6-fluoroindoline replaced by 6-(trifluoromethyl)indoline in Step 7. | 765.1 |
| 48 | 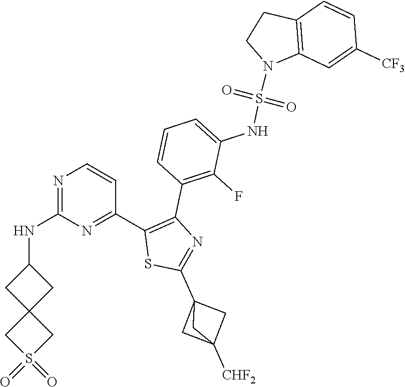 [SMILES: O=S1(=O)CC2(CC(Nc3nccc(-c4sc(C56CC(C(F)F)(C5)C6)nc4-c4cccc(NS(=O)(=O)N5CCc6ccc(C(F)(F)F)cc65)c4F)n3)C2)C1] | 1. bicyclo[1.1.1]pentane-1- carboxylic acid was replaced by 3-(difluoromethyl)bicyclo [1.1.1]pentane-1-carboxylic acid in Step 1 2. 6-fluoroindoline replaced by 6-(trifluoromethyl)indoline in Step 7. | 797.1 |

### Example 49

### Synthesis of N-(3-(2-((1R,5S)-3-oxa-8-azabicyclo[3.2.1]octan-8-yl)-5-(2-((2,2-dioxido-2-thiaspiro[3,3]heptan-6-yl)amino)pyrimidin-4-yl)thiazol-4-yl)-2-fluorophenyl)-4-methoxyisoindoline-2-sulfonamide

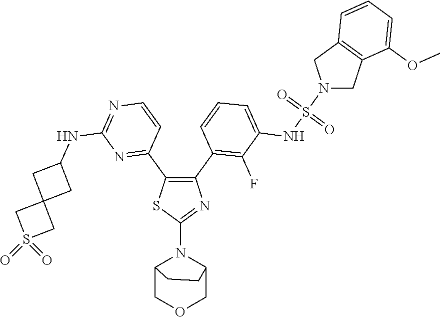 [SMILES: COc1cccc2c1CN(S(=O)(=O)Nc1cccc(-c3nc(N4C5CCC4COC5)sc3-c3ccnc(NC4CC5(C4)CS(=O)(=O)C5)n3)c1F)C2]

### Step 1: N-(3-(2-Amino-5-(2-chloropyrimidin-4-yl)thiazol-4-yl)-2-fluorophenyl)acetamide

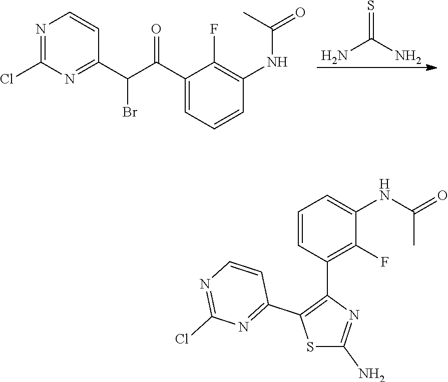

A mixture of N-(3-(2-bromo-2-(2-chloropyrimidin-4-yl)acetyl)-2-fluorophenyl)acetamide (Intermediate 7; 2.50 g, 6.50 mmol, 1.00 eq), thiourea (495 mg, 6.50 mmol, 1.00 eq) in DMA (30.0 ml) was stirred at rt for 30 min, and then at 70° C. overnight. The mixture was concentrated and the residue was purified by silica gel chromatograph on silica gel (DCM/MeOH=30/1) to give the title compound as a yellow solid.

### Step 2: N-(3-(2-Amino-5-(2-(methylthio)pyrimidin-4-yl)thiazol-4-yl)-2-fluorophenyl)acetamide

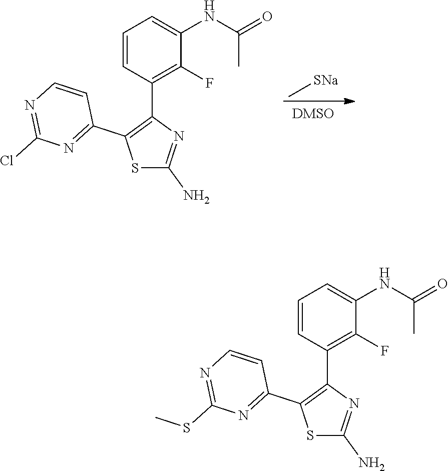

Sodium methanethiolate (96.4 mg, 1.40 mmol, 2.00 eq) was added to a mixture of N-(3-(2-amino-5-(2-chloropyrimidin-4-yl)thiazol-4-yl)-2-fluorophenyl)acetamide (250 mg, 0.69 mmol, 1.00 eq) in DMSO (5.0 ml) at rt and the mixture was stirred overnight. The mixture was diluted with water and extracted with EtOAc. The combined organic layer was washed with brine, dried over Na₂SO₄, filtered, and concentrated to give the title compound as a yellow solid.

### Step 3: N-(3-(2-Bromo-5-(2-(methylthio)pyrimidin-4-yl)thiazol-4-yl)-2-fluorophenyl)acetamide

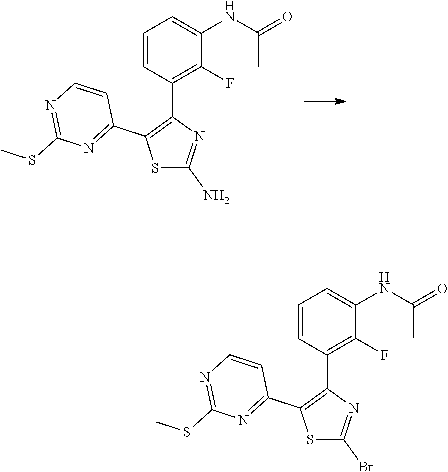 [SMILES: CSc1nccc(-c2sc(N)nc2-c2cccc(NC(C)=O)c2F)n1 ; CSc1nccc(-c2sc(Br)nc2-c2cccc(NC(C)=O)c2F)n1]

tert-Butyl nitrite (92.2 mg, 0.910 mmol, 1.50 eq) was added to a mixture of N-(3-(2-amino-5-(2-(methylthio)pyrimidin-4-yl)thiazol-4-yl)-2-fluorophenyl)acetamide (230 mg, 0.61 mmol, 1.00 eq), CuBr₂ (164 mg, 0.74 mmol, 1.20 eq) in MeCN (5.0 ml) at 0° C. and the mixture was stirred at rt overnight. The mixture was diluted with water and extracted with EtOAc. The combined organic layer was washed with brine, dried over Na₂SO₄, filtered, and then concentrated. The residue was purified by chromatograph on silica gel (DCM/MeOH=50/1) to give the title compound as a yellow solid.

### Step 4: N-(3-(2-(3-Oxa-8-azabicyclo[3.2.1]octan-8-yl)-5-(2-(methylthio)pyrimidin-4-yl)thiazol-4-yl)-2-fluorophenyl)acetamide

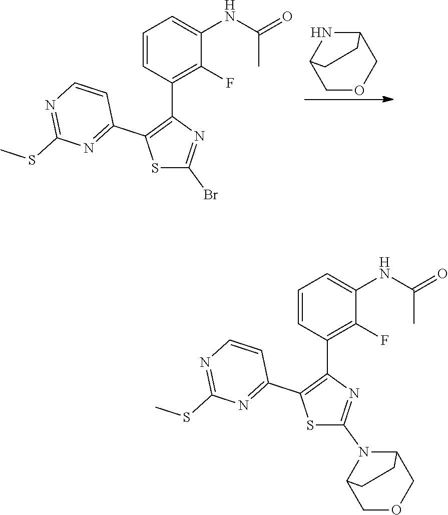

A mixture of N-(3-(2-bromo-5-(2-(methylthio)pyrimidin-4-yl)thiazol-4-yl)-2-fluoro-phenyl)acetamide (200 mg, 0.46 mmol, 1.00 eq), 3-oxa-8-azabicyclo[3.2.1]octane (341 mg, 2.30 mmol, 5.00 eq), TEA (278.8 mg, 2.76 mmol, 6.00 eq) in DMF (2.0 ml) was stirred at 120° C. under microwave irradiation for 3 h. The mixture was diluted with water and extracted with EtOAc. The combined organic layer was washed with brine, dried over Na₂SO₄, filtered, and then concentrated. The residue was purified by chromatograph on silica gel (DCM/MeOH=30/1) to give the title compound as a yellow solid.

### Step 5: 3-(2-(3-Oxa-8-azabicyclo[3.2.1]octan-8-yl)-5-(2-(methylthio)pyrimidin-4-yl)thiazol-4-yl)-2-fluoroaniline

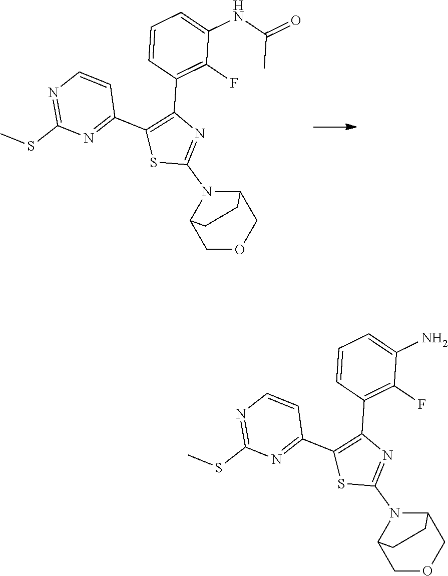 [SMILES: CSc1nccc(-c2sc(N3C4CCC3COC4)nc2-c2cccc(NC(C)=O)c2F)n1 ; CSc1nccc(-c2sc(N3C4CCC3COC4)nc2-c2cccc(N)c2F)n1]

A mixture of N-(3-(2-(3-oxa-8-azabicyclo[3.2.1]octan-8-yl)-5-(2-(methylthio)pyrimidin-4-yl)thiazol-4-yl)-2-fluorophenyl)acetamide (160 mg, 0.34 mmol, 1.00 ea) in HCl/MeOH (2M, 5.0 ml) was stirred at 80° C. for 0.5 h. The mixture was concentrated and diluted with NaHCO₃ aq. and extracted with EtOAc. The organic layer was washed with brine, dried over Na₂SO₄, filtered, and concentrated. The residue was purified by silica gel chromatograph (PE/EA=5/1) to give the title compound as a yellow solid.

### Step 6: N-(3-(2-(3-O) xa-8-azabicyclo[3.2.1]octan-8-yl)-S-(2-(methylthio)pyrimidin-4-yl)thiazol-4-yl)-2-fluorophenyl)-2-oxooxazolidine-3-sulfonamide

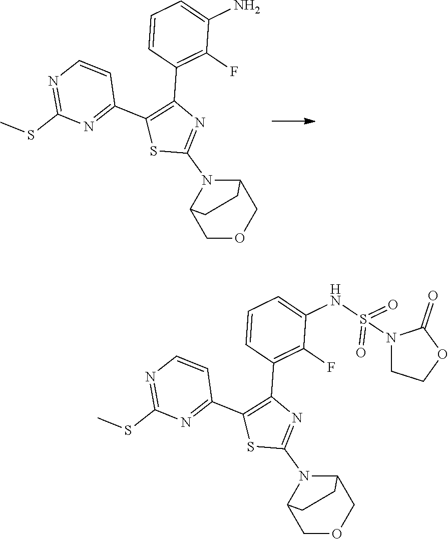 [SMILES: CSc1nccc(-c2sc(N3C4CCC3COC4)nc2-c2cccc(N)c2F)n1 ; CSc1nccc(-c2sc(N3C4CCC3COC4)nc2-c2cccc(NS(=O)(=O)N3CCOC3=O)c2F)n1]

2-Bromoethanol (84.9 mg, 0.69 mmol, 2.00 eq) was added to a mixture of sulfurisocyanatidic chloride (96.6 mg, 0.69 mmol, 2.00 eq) in DCM (10 ml) at 0° C. and the mixture was stirred at 0° C. for 20 min. The mixture was added to a solution of 3-(2-(3-oxa-8-azabicyclo[3.2.1]-octan-8-yl)-5-(2-(methylthio)pyrimidin-4-yl)thiazol-4-yl)-2-fluoroaniline (160 mg, 0.34 mmol, 1.00 eq) and TEA (172 mg, 1.70 mmol, 5.00 eq) in DCM (3.0 ml) at 0° C. and the resulting mixture was stirred at rt overnight. The mixture was concentrated and the residue was purified by chromatograph on silica gel (DCM/MeOH=20/1) to give the title compound as a yellow solid.

### Step 7: N-(3-(2-(3-Oxa-8-azabicyclo[3.2.1]octan-8-yl)-5-(2-(methylthio)pyrimidin-4-yl)thiazol-4-yl)-2-fluorophenyl)-4-methoxyisoindoline-2-sulfonamide

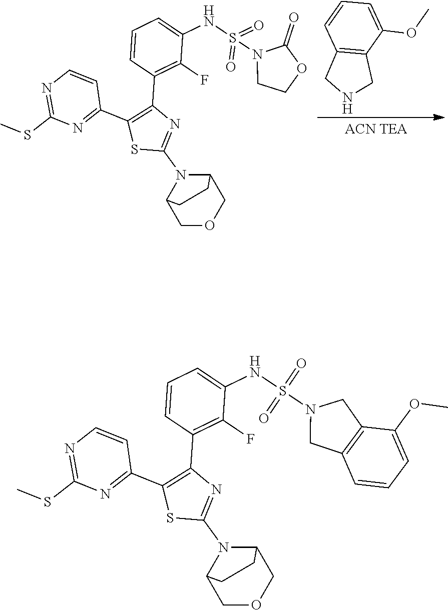

A mixture of N-(3-(2-(3-oxa-8-azabicyclo[3.2.1]octan-8-yl)-5-(2-(methylthio)pyrimidin-4-yl)thiazol-4-yl)-2-fluorophenyl)-2-oxooxazolidine-3-sulfonamide (100 mg, 0.17 mmol, 1.00 eq), 4-methoxyisoindoline hydrochloride (47.2 mg, 0.26 mmol, 1.50 eq), TEA (51.5 mg, 0.51 mmol, 3.00 eq) in MeCN (2.0 ml) was stirred at 100° C. under microwave irradiation. The mixture was concentrated and the residue was purified by chromatograph on silica gel (DCM/MeOH=30/1) to give the title compound as a yellow solid.

### Step 8: N-(3-(2-(3-Oxa-8-azabicyclo[3.2.1]octan-8-yl)-5-(2-(methylsulfonyl)pyrimidin-4-yl)-thiazol-4-yl)-2-fluorophenyl)-4-methoxyisoindoline-2-sulfonamide

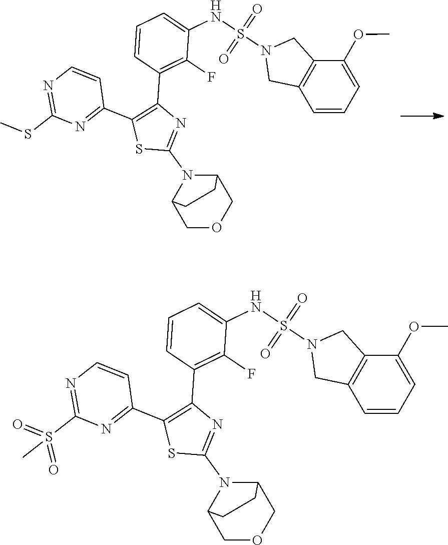

Oxone (86.3 mg, 0.15 mmol 3.00 eq) was added to a mixture of N-(3-(2-(3-oxa-8-azabicyclo[3.2.1]octan-8-yl)-5-(2-(methylthio)pyrimidin-4-yl)thiazol-4-yl)-2-fluorophenyl)-4-methoxyisoindoline-2-sulfonamide (30 mg, 0.05 mmol, 1.00 eq) in THF/MeOH/H₂O=2/2/1 (2.0 ml) at rt and the mixture was stirred overnight. The mixture was diluted with water and extracted with EtOAc. The combined organic layer was washed with brine, dried over Na₂SO₄, filtered, and concentrated to give the title compound as a yellow solid.

### Step 9: N-(3-(2-(3-Oxa-8-azabicyclo[3.2.1]octan-8-yl)-5-(2-((2,2-dioxido-2-thiaspiro[3.3]heptan-6-yl)amino)pyrimidin-4-yl)thiazol-4-yl)-2-fluorophenyl)-4-methoxyisoindoline-2-sulfonamide

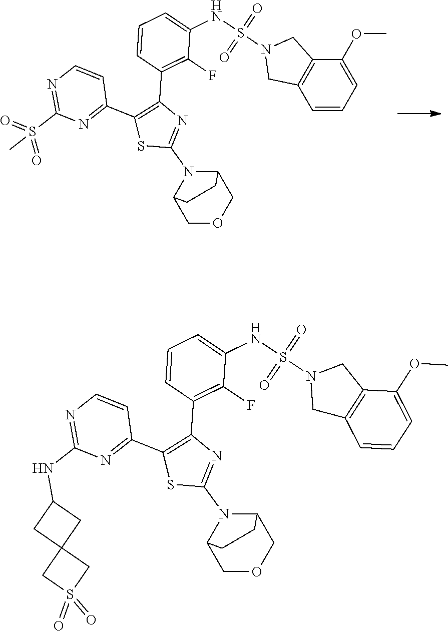 [SMILES: COc1cccc2c1CN(S(=O)(=O)Nc1cccc(-c3nc(N4C5CCC4COC5)sc3-c3ccnc(S(C)(=O)=O)n3)c1F)C2 ; COc1cccc2c1CN(S(=O)(=O)Nc1cccc(-c3nc(N4C5CCC4COC5)sc3-c3ccnc(NC4CC5(C4)CS(=O)(=O)C5)n3)c1F)C2]

A mixture of N-(3-(2 (3-oxa-8-azabicyclo[3.2.1]octan-8-yl)-5-(2-(methylsulfonyl)-pyrimidin-4-yl)thiazol-4-yl)-2-fluorophenyl)-4-methoxyisoindoline-2-sulfonamide (20 mg, 0.03 mmol, 1.00 eq), 6-amino-2-thiaspiro[3.3]heptane 2,2-dioxide (Intermediate 6; 7.3 mg, 0.045 mmol, 1.50 eq), DIEA (11.6 mg, 0.09 mmol, 3.00 eq) in DMSO (0.5 ml) was stirred at 70° C. overnight. The mixture was diluted with water and extracted with EtOAc. The organic layer was washed with brine, dried over Na₂SO₄, filtered, and concentrated. The residue was purified by prep-HPLC to give the title compound as a yellow solid. LCMS (ES, m/z): [M+H]⁺=754.2.

Proceeding analogously as described in Example 49, the following compounds were prepared.

| | | | LCMS (ES, |
|---|---|---|---|
| | | | m/z): |
| Ex# | Compound | Synthesis | [M + H]⁺ |
| 50 | 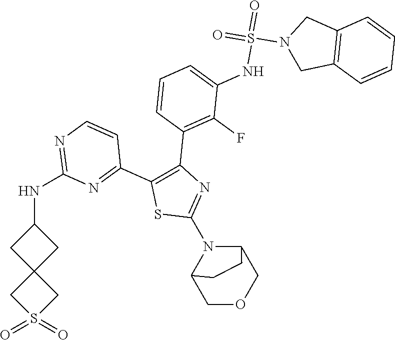 [SMILES: O=S1(=O)CC2(CC(Nc3nccc(-c4sc(N5C6CCC5COC6)nc4-c4cccc(NS(=O)(=O)N5Cc6ccccc6C5)c4F)n3)C2)C1] | 4-methoxyisoindoline replaced by isoindoline in Step 7. | 724.2 |
| 51 | 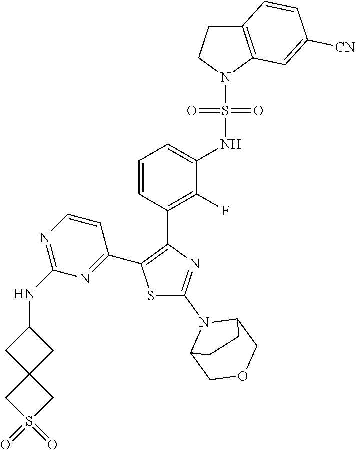 [SMILES: N#Cc1ccc2c(c1)N(S(=O)(=O)Nc1cccc(-c3nc(N4C5CCC4COC5)sc3-c3ccnc(NC4CC5(C4)CS(=O)(=O)C5)n3)c1F)CC2] | 4-methoxyisoindoline replaced by indoline-6-carbonitrile in Step 7. | 749.2 |
| 52 | 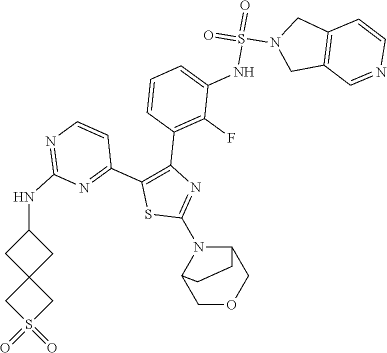 [SMILES: O=S1(=O)CC2(CC(Nc3nccc(-c4sc(N5C6CCC5COC6)nc4-c4cccc(NS(=O)(=O)N5Cc6ccncc6C5)c4F)n3)C2)C1] | 4-methoxyisoindoline replaced by 2,3-dihydro-1H-pyrrolo[3,4- c]pyridine in Step 7. | 725.2 |

### Example 53

### Synthesis of N-(3-(5-(2-((2,2-dioxido-2-thiaspiro[3.3]heptan-6-yl)amino)pyrimidin-4-yl)-2-(3-methyl-3,8-diazabicyclo[3.2.1]octan-8-yl)thiazol-4-yl)-2-fluorophenyl)-4-methoxyisoindoline-2-sulfonamide

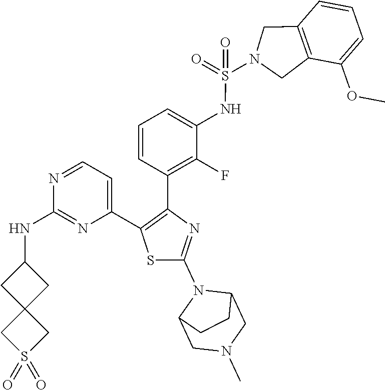 [SMILES: COc1cccc2c1CN(S(=O)(=O)Nc1cccc(-c3nc(N4C5CCC4CN(C)C5)sc3-c3ccnc(NC4CC5(C4)CS(=O)(=O)C5)n3)c1F)C2]

### Step 1: N-(2-Fluoro-3-(2-(3-methyl-3,8-diazabicyclo[3.2.1]octan-8-yl)-5-(2-(methylthio)-pyrimidin-4-yl)thiazol-4-yl)phenyl)acetamide

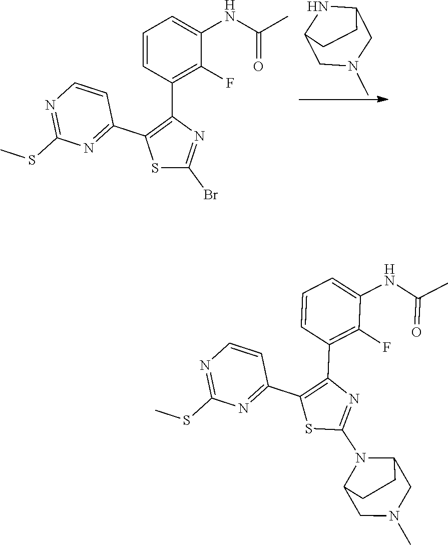

A mixture of N-(3-(2-bromo-5-(2-(methylthio)pyrimidin-4-yl)thiazol-4-yl)-2-fluoro-phenyl)acetamide (500 mg, 1.14 mmol, 1.00 eq), 3-methyl-3,8-diazabicyclo[3.2.1]octane hydrochloride (279 mg, 1.71 mmol, 1.50 eq), and TEA (693 mg, 6.85 mmol, 6.00 eq) in DMA (5.0 ml) was stirred at 120° C. for 3 h under microwave irradiation. The mixture was concentrated and the residue was purified by flash column chromatography (EA:PE=0 to 100%) to give the title compound as a yellow oil.

### Step 2: N-(2-Fluoro-3-(2-(3-methyl-3,8-diazabicyclo[3.2.1]octan-8-yl)-5-(2-(methylsulfonyl)-pyrimidin-4-yl)thiazol-4-yl)phenyl)acetamide

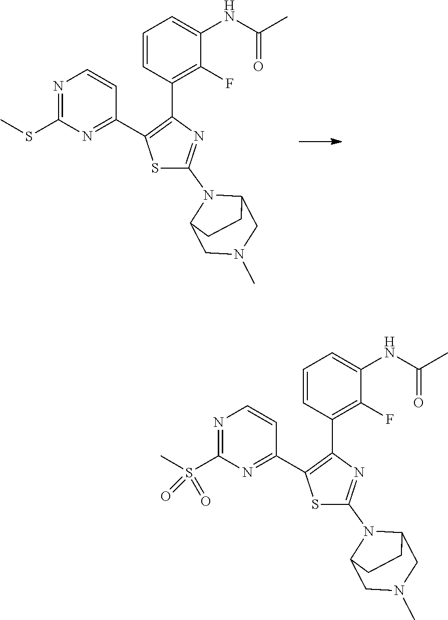

Oxone (3.50 g, 5.70 mmol, 3.00 eq.) was added to a stirring solution of N-(2-fluoro-3-(2-(3-methyl-3,8-diazabicyclo[3.2.1]octan-8-yl)-5-(2-(methylthio)pyrimidin-4-yl)thiazol-4-yl)-phenyl)acetamide (0.92 g, 1.90 mmol, 1.00 eq.) in MeOH:THF:H₂O)=2:2:1 (30.0 mL) and the mixture was stirred at rt overnight. The mixture was filtered, and the solid was rinsed with DCM. The organic layer was concentrated to give the title compound as a yellow solid.

### Step 3: N-(3-(5-(2-((2,2-Dioxido-2-thiaspiro[3.3]heptan-6-yl)amino)pyrimidin-4-yl)-2-(3-methyl-3,8-diazabicyclo[3.2.1]octan-8-yl)thiazol-4-yl)-2-fluorophenyl)acetamide

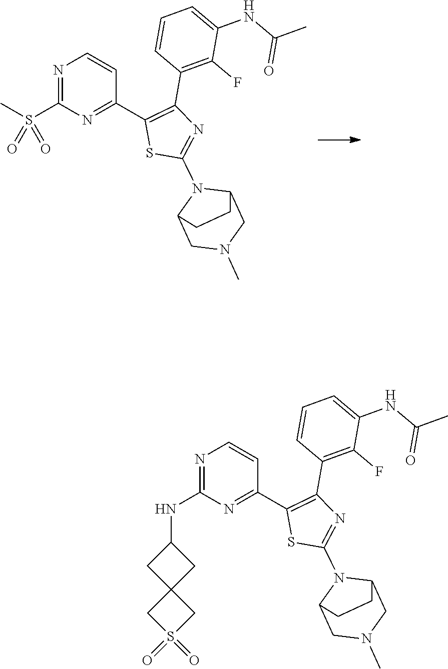 [SMILES: CC(=O)Nc1cccc(-c2nc(N3C4CCC3CN(C)C4)sc2-c2ccnc(S(C)(=O)=O)n2)c1F ; CC(=O)Nc1cccc(-c2nc(N3C4CCC3CN(C)C4)sc2-c2ccnc(NC3CC4(C3)CS(=O)(=O)C4)n2)c1F]

6-Amino-2-thiaspiro[3.3]heptane 2,2-dioxide (Intermediate 6; 0.56 g, 2.80 mmol, 1.50 eq.) and DIEA (0.73 g, 5.70 mmol, 3.00 eq.) were added to a stirred mixture of N-(2-fluoro-3-(2-(3-methyl-3,8-diazabicyclo[3.2.1]octan-8-yl)-5-(2-(methylsulfonyl)pyrimidin-4-yl)thiazol-4-yl)phenyl)acetamide (0.98 g, 1.90 mmol, 1.00 eq.) in DMSO (10.0 mL) and the mixture was stirred at 70° C. under Na overnight. The mixture was poured into water, extracted with EtOAc, and the combined organic layers were washed with water, brine, dried over Na₂SO₄, filtered, and concentrated to give the title compound as a yellow solid.

### Step 4: 6-(4-(4-(3-Amino-2-fluorophenyl)-2-(3-methyl-3,8-diazabicyclo[3.2.1]octan-8-yl)thiazol-5-yl)pyrimidin-2-yl)amino)-2-thiaspiro[3.3]heptane 2,2-dioxide

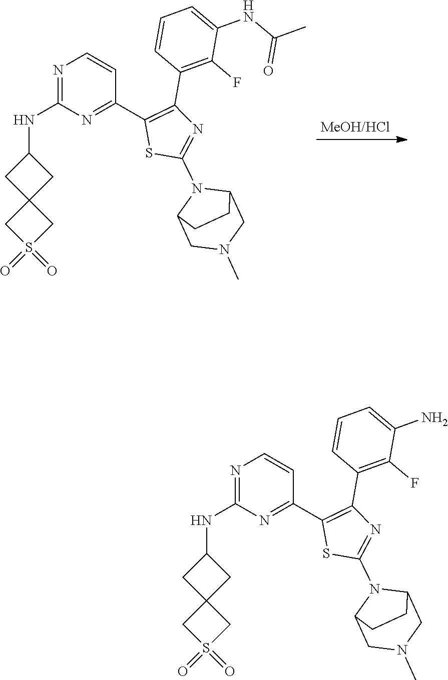 [SMILES: CC(=O)Nc1cccc(-c2nc(N3C4CCC3CN(C)C4)sc2-c2ccnc(NC3CC4(C3)CS(=O)(=O)C4)n2)c1F ; CN1CC2CCC(C1)N2c1nc(-c2cccc(N)c2F)c(-c2ccnc(NC3CC4(C3)CS(=O)(=O)C4)n2)s1]

MeOH/HCl (2M, 5.0 mL) was added to N-(3-(5-(2-((2,2-dioxido-2-thiaspiro[3.3]heptan-6-yl)amino)pyrimidin-4-yl)-2-(3-methyl-3,8-diazabicyclo[3.2.1]octan-8-yl)thiazol-4-yl)-2-fluoro-phenyl)acetamide (0.60 g, 1.00 mmol) in MeOH (5.0 mL) and the mixture was stirred at 50° C. for 2 h under N₂. The mixture was concentrated and 1N NaOH aq. was added. The mixture was extracted with EtOAc, and the combined organic layers were washed with water, brine, dried over Na₂SO₄, filtered, and concentrated to give the title compound as a yellow solid.

### Step 5: N-(3-(5-(2-((2,2-Dioxido-2-thiaspiro[3.3]heptan-6-yl)amino)pyrimidin-4-yl)-2-(3-methyl-3,8-diazabicyclo[3.2.1]octan-8-yl)thiazol-4-yl)-2-fluorophenyl)-2-oxooxazolidine-3-sulfonamide

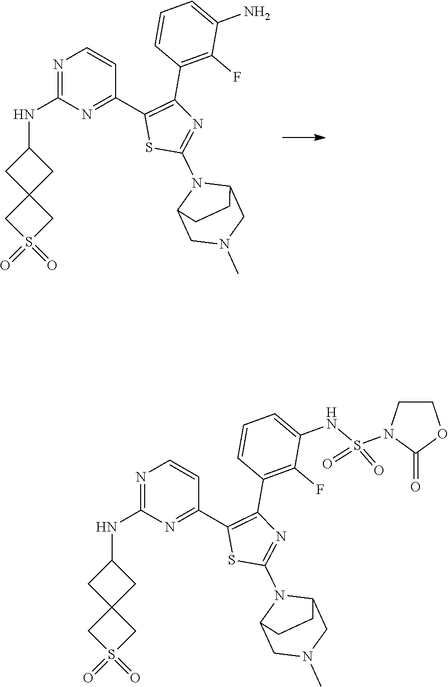 [SMILES: CN1CC2CCC(C1)N2c1nc(-c2cccc(N)c2F)c(-c2ccnc(NC3CC4(C3)CS(=O)(=O)C4)n2)s1 ; CN1CC2CCC(C1)N2c1nc(-c2cccc(NS(=O)(=O)N3CCOC3=O)c2F)c(-c2ccnc(NC3CC4(C3)CS(=O)(=O)C4)n2)s1]

2-Bromoethan-1-ol (0.20 g, 1.66 mmol, 2.00 eq) was added to a solution of sulfurisocyanatidic chloride (0.23 g, 1.66 mmol, 2.00 eq) in DCM (10 mL) at 0° C. and the mixture was stirred at 0° C. for 1 h. The mixture was added to a solution of 6-((4-(4-(3-amino-2-fluorophenyl)-2-(3-methyl-3,8-diazabicyclo[3.2.1]octan-8-yl)thiazol-S-yl)pyrimidin-2-yl)amino)-2-thiaspiro-[3.3]heptane 2,2-dioxide (0.46 g, 0.83 mmol, 1.00 eq) and TEA (0.42 g, 4.14 mmol, 5.0 eq) in DCM (5.0 mL) at 0° C. and the mixture was stirred at r.t. for 8 h under N₂. The mixture was concentrated and the residue was purified by flash column chromatography (EA:PE=0 to 100%) to give the title compound as a yellow solid.

### Step 6: N-(3-(5-(2-((2,2-Dioxido-2-thiaspiro[3.3]heptan-6-yl)amino)pyrimidin-4-yl)-2-(3-methyl-3,8-diazabicyclo[3.2.1]octan-8-yl)thiazol-4-yl)-2-fluorophenyl)-4-methoxyisoindoline-2-sulfonamide

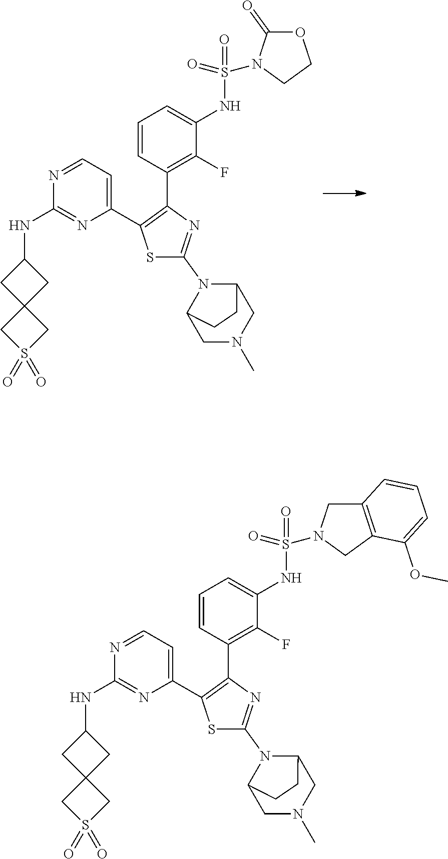 [SMILES: CN1CC2CCC(C1)N2c1nc(-c2cccc(NS(=O)(=O)N3CCOC3=O)c2F)c(-c2ccnc(NC3CC4(C3)CS(=O)(=O)C4)n2)s1 ; COc1cccc2c1CN(S(=O)(=O)Nc1cccc(-c3nc(N4C5CCC4CN(C)C5)sc3-c3ccnc(NC4CC5(C4)CS(=O)(=O)C5)n3)c1F)C2]

4-Methoxyisoindoline (32 mg, 0.17 mmol, 1.50 eq.) was added to a solution of N-(3-(5-(2-((2,2-dioxido-2-thiaspiro[3.3]heptan-6-yl)amino)pyrimidin-4-yl)-2-(3-methyl-3,8-diazabicyclo-[3.2.1]octan-8-yl)thiazol-4-yl)-2-fluorophenyl)-2-oxooxazolidine-3-sulfonamide (80 mg, 0.11 mmol, 1.00 eq.) in MeCN (2.0 mL) and TEA (69 mg, 0.68 mmol, 6.00 eq.) and the mixture was stirred at 100° C. for 3 h under microwave irradiation. The mixture was concentrated and the residue was purified by prep-HPLC to give the title compound as a pale yellow solid. MS (ES, m/z): [M+1]⁺=767.3.

Proceeding analogously as described in Example 53, the following compounds were prepared.

| | | | LCMS (ES. |
|---|---|---|---|
| | | | m/z): |
| Ex# | Compound | Synthesis | [M + 1]⁺ |
| 54 | 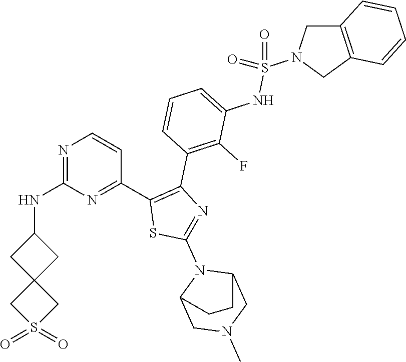 [SMILES: CN1CC2CCC(C1)N2c1nc(-c2cccc(NS(=O)(=O)N3Cc4ccccc4C3)c2F)c(-c2ccnc(NC3CC4(C3)CS(=O)(=O)C4)n2)s1] | 4-methoxyisoindoline replaced by isoindoline in Step 6. | 737.2 |
| 55 | 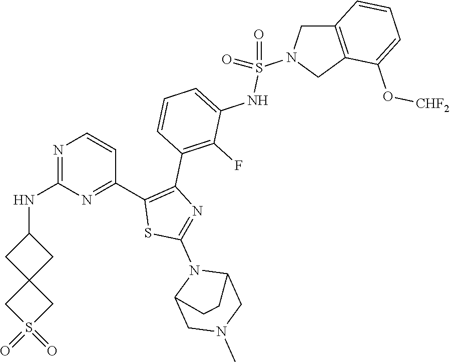 [SMILES: CN1CC2CCC(C1)N2c1nc(-c2cccc(NS(=O)(=O)N3Cc4cccc(OC(F)F)c4C3)c2F)c(-c2ccnc(NC3CC4(C3)CS(=O)(=O)C4)n2)s1] | 4-methoxyisoindoline replaced by Intermediate 11 in Step 6. | 803.3 |
| 56 | 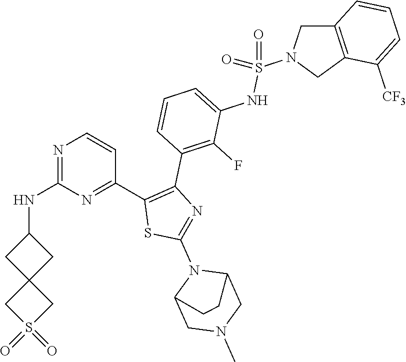 [SMILES: CN1CC2CCC(C1)N2c1nc(-c2cccc(NS(=O)(=O)N3Cc4cccc(C(F)(F)F)c4C3)c2F)c(-c2ccnc(NC3CC4(C3)CS(=O)(=O)C4)n2)s1] | 4-methoxyisoindoline replaced by 4- (trifluoromethyl)isoindoline in Step 6. | 805.3 |
| 57 | 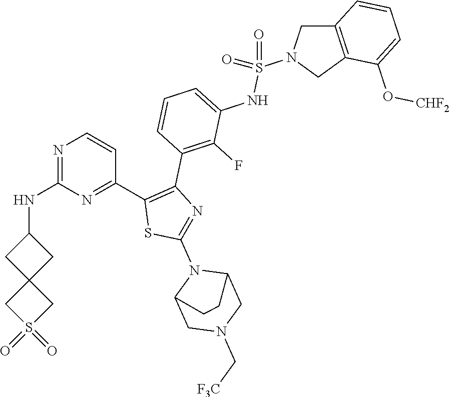 [SMILES: O=S1(=O)CC2(CC(Nc3nccc(-c4sc(N5C6CCC5CN(CC(F)(F)F)C6)nc4-c4cccc(NS(=O)(=O)N5Cc6cccc(OC(F)F)c6C5)c4F)n3)C2)C1] | 1. 3-methyl-3,8-diazabicyclo- [3.2.1]octane replaced by Intermediate 19 in Step 1. 2. 4-methoxyisoindoline replaced by Intermediate 11 in Step 6. | 871.3 |
| 58 | 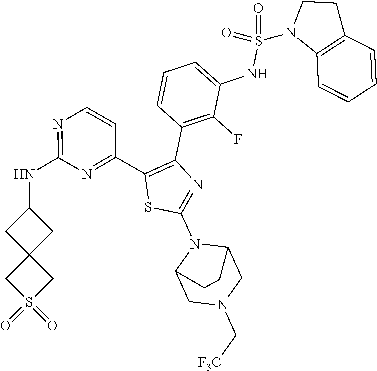 [SMILES: O=S1(=O)CC2(CC(Nc3nccc(-c4sc(N5C6CCC5CN(CC(F)(F)F)C6)nc4-c4cccc(NS(=O)(=O)N5CCc6ccccc65)c4F)n3)C2)C1] | 1. 3-methyl-3,8-diazabicyclo- [3.2.1]octane replaced by Intermediate 19 in Step 1. 2. 4-methoxyisoindoline replaced by indoline in Step 6. | 805.3 |
| 59 | 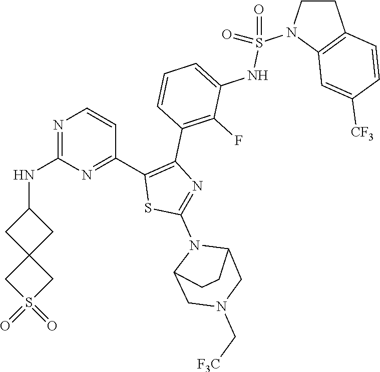 [SMILES: O=S1(=O)CC2(CC(Nc3nccc(-c4sc(N5C6CCC5CN(CC(F)(F)F)C6)nc4-c4cccc(NS(=O)(=O)N5CCc6ccc(C(F)(F)F)cc65)c4F)n3)C2)C1] | 1. 3-methyl-3,8-diazabicyclo- [3.2.1]octane replaced by Intermediate 19 in Step 1. 2. 4-methoxyisoindoline replaced by 6-(trifluoromethyl)indoline in Step 6. | 873.3 |
| 60 | 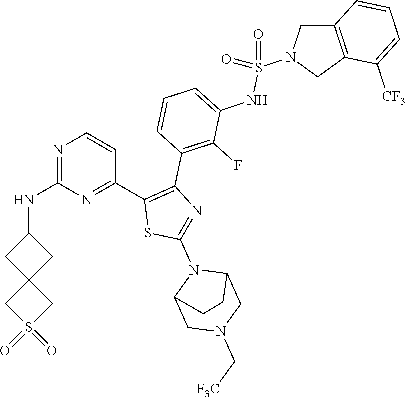 [SMILES: O=S1(=O)CC2(CC(Nc3nccc(-c4sc(N5C6CCC5CN(CC(F)(F)F)C6)nc4-c4cccc(NS(=O)(=O)N5Cc6cccc(C(F)(F)F)c6C5)c4F)n3)C2)C1] | 1. 3-methyl-3,8-diazabicyclo- [3.2.1]octane replaced by Intermediate 19 in Step 1. 2. 4-methoxyisoindoline replaced by 4-(trifluoromethyl)- isoindoline in Step 6. | 873.2 |
| 61 | 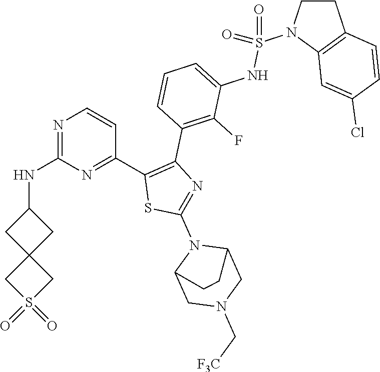 [SMILES: O=S1(=O)CC2(CC(Nc3nccc(-c4sc(N5C6CCC5CN(CC(F)(F)F)C6)nc4-c4cccc(NS(=O)(=O)N5CCc6ccc(Cl)cc65)c4F)n3)C2)C1] | 1. 3-methyl-3,8- diazabicyclo[3.2.1]octane replaced by Intermediate 19 in Step 1. 2. 4-methoxyisoindoline replaced by 6-chloroindoline in Step 6. | 839.2 |
| 62 | 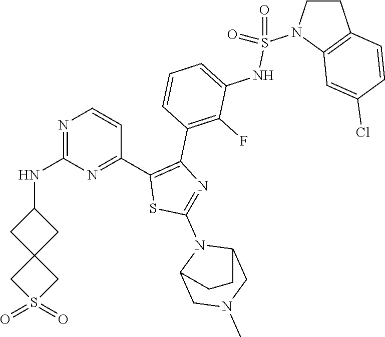 [SMILES: CN1CC2CCC(C1)N2c1nc(-c2cccc(NS(=O)(=O)N3CCc4ccc(Cl)cc43)c2F)c(-c2ccnc(NC3CC4(C3)CS(=O)(=O)C4)n2)s1] | 4-methoxyisoindoline replaced by 6- chloroindoline in Step 6. | 771.3 |
| 63 | 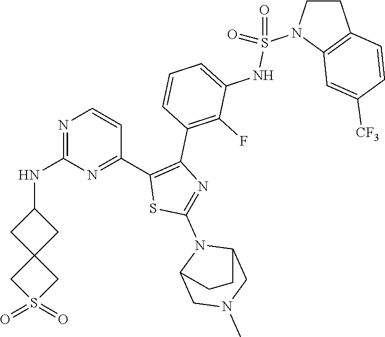 [SMILES: CN1CC2CCC(C1)N2c1nc(-c2cccc(NS(=O)(=O)N3CCc4ccc(C(F)(F)F)cc43)c2F)c(-c2ccnc(NC3CC4(C3)CS(=O)(=O)C4)n2)s1] | 4-methoxyisoindoline replaced by 6- (trifluoromethyl)indoline in Step 6. | 805.3 |
| 64 | 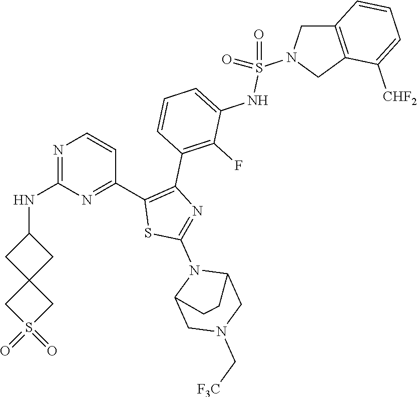 [SMILES: O=S1(=O)CC2(CC(Nc3nccc(-c4sc(N5C6CCC5CN(CC(F)(F)F)C6)nc4-c4cccc(NS(=O)(=O)N5Cc6cccc(C(F)F)c6C5)c4F)n3)C2)C1] | 1. 3-methyl-3,8- diazabicyclo[3.2.1]octane replaced by Intermediate 19 in Step 1. 2. 4-methoxyisoindoline replaced by Intermediate 8 in Step 6. | 855.3 |
| 65 | 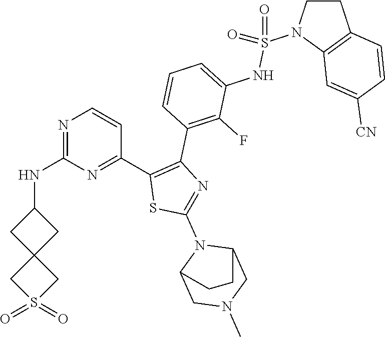 [SMILES: CN1CC2CCC(C1)N2c1nc(-c2cccc(NS(=O)(=O)N3CCc4ccc(C#N)cc43)c2F)c(-c2ccnc(NC3CC4(C3)CS(=O)(=O)C4)n2)s1] | 4-methoxyisoindoline replaced by indoline-6-carbonitrile in Step 6. | 762.3 |
| 66 | 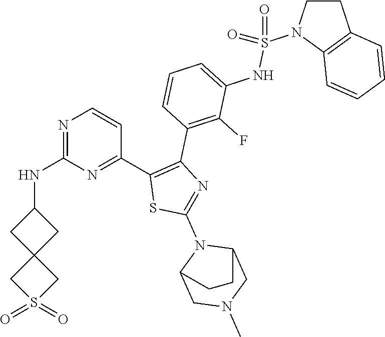 [SMILES: CN1CC2CCC(C1)N2c1nc(-c2cccc(NS(=O)(=O)N3CCc4ccccc43)c2F)c(-c2ccnc(NC3CC4(C3)CS(=O)(=O)C4)n2)s1] | 4-methoxyisoindoline replaced by indoline in Step 6. | 737.2 |
| 67 | 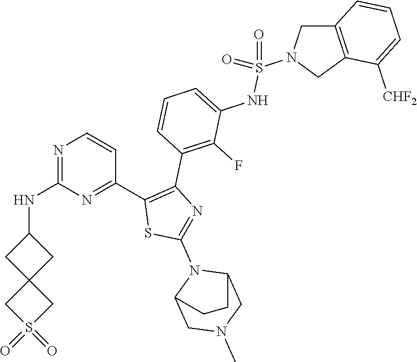 [SMILES: CN1CC2CCC(C1)N2c1nc(-c2cccc(NS(=O)(=O)N3Cc4cccc(C(F)F)c4C3)c2F)c(-c2ccnc(NC3CC4(C3)CS(=O)(=O)C4)n2)s1] | 4-methoxyisoindoline replaced by Intermediate 8 in Step 9. | 787.2 |

### Example 68

### Synthesis of N-(2-fluoro-3-(5-(2-(((1r,4r)-4-(methylsulfonyl)cyclohexyl)-amino)pyrimidin-4-yl)-2-(3-(trifluoromethyl)bicyclo[1.1.1]pentan-1-yl)thiazol-4-yl)phenyl)-indoline-1-sulfonamide

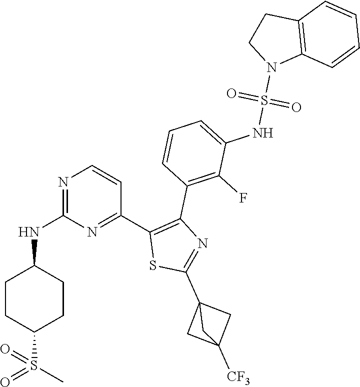 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccccc54)c3F)n2)CC1]

### Step 1:3-(Trifluoromethyl)bicyclo[1.1.1]pentane-1-carboxamide

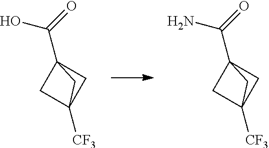 [SMILES: O=C(O)C12CC(C(F)(F)F)(C1)C2 ; NC(=O)C12CC(C(F)(F)F)(C1)C2]

Oxalyl chloride (641 mg, 5.05 mmol, 1.30 eq.) and DMF (1 drop) were added to a mixture of 3-(trifluoromethyl)bicyclo[1.1.1]pentane-1-carboxylic acid (700 mg, 3.89 mmol, 1.00 eq) in DCM (14.0 mL) dropwise at 0° C. and the mixture was stirred at r.t. under Na for 2 h. NHs (gas) was bubbled into the reaction mixture at −10° C. This reaction was warmed to r.t. and stirred overnight. The mixture was filtered, and the filtrate was concentrated to give the crude product as a white solid, which was used in the next step.

### Step 2:3-(Trifluoromethyl)bicyclo[1.1.1]pentane-1-carbothioamide

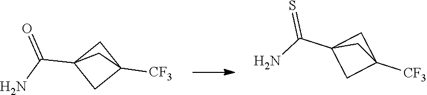 [SMILES: NC(=O)C12CC(C(F)(F)F)(C1)C2 ; NC(=S)C12CC(C(F)(F)F)(C1)C2]

Lawesson's reagent (1.57 g, 3.89 mmol, 1.00 eq.) was added to a mixture of (trifluoromethyl)bicyclo[1.1.1]pentane-1-carboxamide (697 mg, 3.89 mmol, 1.00 eq) in THF (28.0 mL) and the mixture was stirred at 65° C. under Na overnight. The mixture was concentrated and the residue was purified by column chromatography on silica gel (PE:EA=5:1) to give the title compound as a white solid.

### Step 3: N-(3-(5-(2-Chloropyrimidin-4-yl)-2-(3-(trifluoromethyl)-bicyclo[1.1.1]pentan-1-yl)thiazol-4-yl)-2-fluorophenyl)acetamide

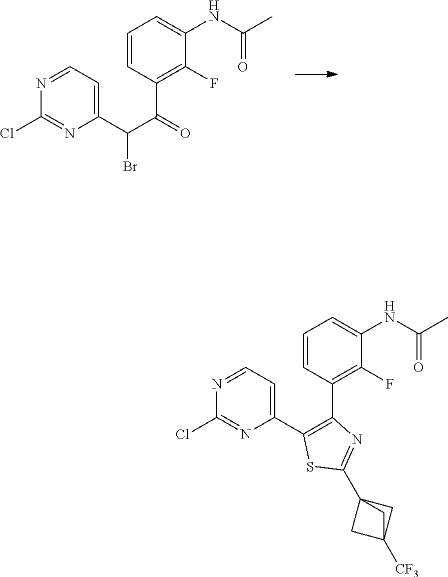 [SMILES: CC(=O)Nc1cccc(C(=O)C(Br)c2ccnc(Cl)n2)c1F ; CC(=O)Nc1cccc(-c2nc(C34CC(C(F)(F)F)(C3)C4)sc2-c2ccnc(Cl)n2)c1F]

A mixture of N-(3-(2-bromo-2-(2-chloropyrimidin-4-yl)acetyl)-2-fluorophenyl)acetamide (3.00 g, 7.76 mmol, 1.00 eq), 3-(trifluoromethyl)bicyclo[1.1.1]pentane-1-carbothioamide (1.51 g, 7.76 mmol, 1.00 eq) in DMA (40.0 mL) was stirred at 70° C. for 16 h under N₂. The mixture was poured into water and extracted with EtOAc. The combined organic layers were washed with water, brine, dried over Na₂SO₄, filtered and concentrated. The residue was purified by flash column chromatography (EA:PE=0 to 100%) to give the title compound as a yellow solid.

### Step 4: N-(2-Fluoro-3-(5-(2-(((1r,4r)-4-(methylsulfonyl)cyclohexyl)amino)-pyrimidin-4-yl)-2-(3-(trifluoromethyl)bicyclo[1.1.1]pentan-1-yl)thiazol-4-yl)phenyl)acetamide

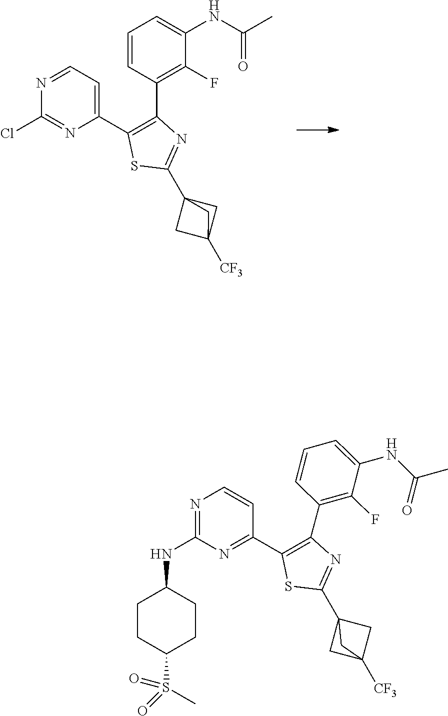 [SMILES: CC(=O)Nc1cccc(-c2nc(C34CC(C(F)(F)F)(C3)C4)sc2-c2ccnc(Cl)n2)c1F ; CC(=O)Nc1cccc(-c2nc(C34CC(C(F)(F)F)(C3)C4)sc2-c2ccnc(N[C@H]3CC[C@H](S(C)(=O)=O)CC3)n2)c1F]

A mixture of N-(3-(5-(2-chloropyrimidin-4-yl)-2-(3-(trifluoromethyl)-bicyclo[1.1.1]pentan-1-yl)thiazol-4-yl)-2-fluorophenyl)acetamide (1.00 g, 2.07 mmol, 1.00 eq.), (1s,4s)-4-(methyl-sulfonyl)cyclohexanamine (Intermediate 5; 681 mg, 2.48 mmol, 1.20 eq), Cs₂CO₃ (3.37 g, 10.35 mmol, 5.00 eq), RuPhos (300 mg), and RuPhos Pd G2 (300 mg) in n-BuOH (10.0 mL) and NMP (1.0 mL) was stirred at 90° C. for 20 h under N₂. The mixture was poured into water and extracted with EtOAc. The combined organic layers were washed with water, brine, dried over Na₂SO₄, filtered, and concentrated. The residue was purified by flash column chromatography (EA:PE=0 to 100%) to give the title compound as a yellow solid.

### Step 5: 4-(4-(3-Amino-2-fluorophenyl)-2-(3-(trifluoromethyl)-bicyclo[1.1.1]pentan-1-yl)thiazol-5-yl)-N-((1r,4r)-4-(methylsulfonyl)cyclohexyl)pyrimidin-2-amine

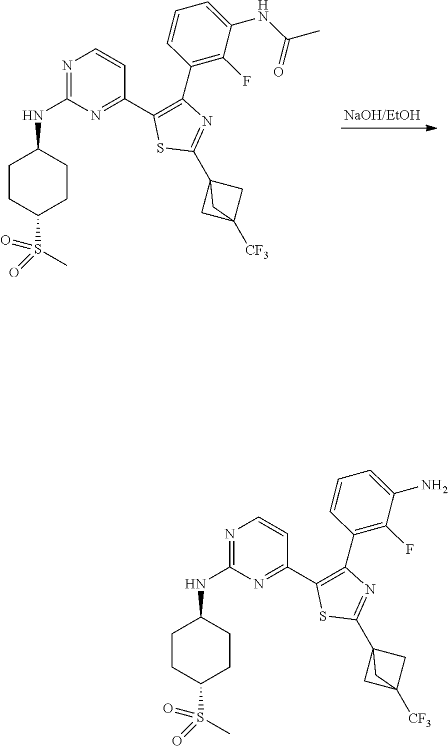 [SMILES: CC(=O)Nc1cccc(-c2nc(C34CC(C(F)(F)F)(C3)C4)sc2-c2ccnc(N[C@H]3CC[C@H](S(C)(=O)=O)CC3)n2)c1F ; CS(=O)(=O)[C@H]1CC[C@H](Nc2nccc(-c3sc(C45CC(C(F)(F)F)(C4)C5)nc3-c3cccc(N)c3F)n2)CC1]

NaOH (125 mg, 3.13 mmol, 3.00 eq.) was added to a solution of N-(2-fluoro-3-(5-(2-(((1r,4r)-4-(methylsulfonyl)cyclohexyl)amino)pyrimidin-4-yl)-2-(3-(trifluoromethyl)bicyclo-[1.1.1]pentan-1-yl)thiazol-4-yl)phenyl)acetamide (650 mg, 1.04 mmol, 1.00 eq.) in EtOH (5.0 mL) and the mixture was stirred at 70° C. for 30 h under N₂. The mixture was diluted with water and extracted with EtOAc. The combined organic layers were washed with water, brine, dried over Na₂SO₄, filtered, and concentrated. The residue was purified by flash column chromatography (MeOH:DCM=0 to 10%) to give the title compound as a yellow solid.

### Step 6: N-(2-Fluoro-3-(5-(2-(((1r,4r)-4-(methylsulfonyl)cyclohexyl)-amino)pyrimidin-4-yl)-2-(3-(trifluoromethyl)bicyclo[1.1.1]pentan-1-yl)thiazol-4-yl)phenyl)-2-oxooxazolidine-3-sulfonamide

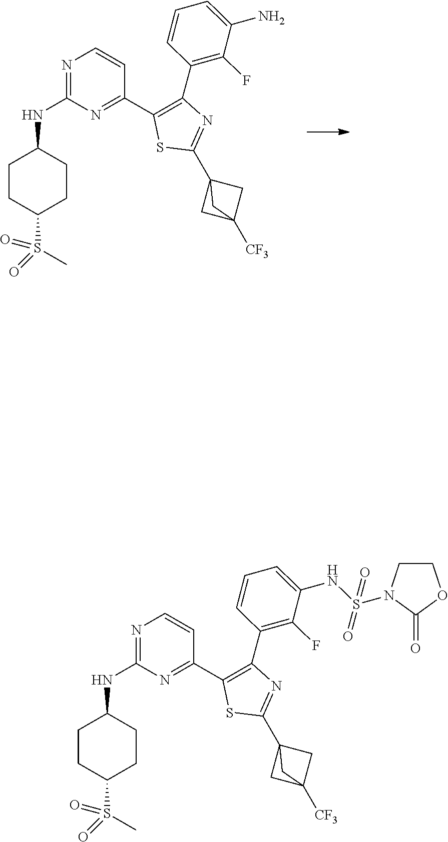 [SMILES: CS(=O)(=O)[C@H]1CC[C@H](Nc2nccc(-c3sc(C45CC(C(F)(F)F)(C4)C5)nc3-c3cccc(N)c3F)n2)CC1 ; CS(=O)(=O)[C@H]1CC[C@H](Nc2nccc(-c3sc(C45CC(C(F)(F)F)(C4)C5)nc3-c3cccc(NS(=O)(=O)N4CCOC4=O)c3F)n2)CC1]

To a solution of sulfurisocyanatidic chloride (241 mg, 1.72 mmol, 2.00 eq) in DCM (15.0 mL) was added 2-bromoethan-1-ol (212 mg, 1.72 mmol, 2.00 eq) at 0° C. and the mixture was stirred at 0° C. for 1 h under N₂. A solution of 4-(4-(3-amino-2-fluorophenyl)-2-(3-(trifluoro-methyl)bicyclo[1.1.1]pentan-1-yl)thiazol-5-yl)-N-((1r,4r)-4-(methylsulfonyl)-cyclohexyl)-pyrimidin-2-amine (500 mg, 0.86 mmol, 1.00 eq) in DCM (15.0 mL) and TEA (348 mg, 3.44 mmol, 4.00 eq) was added to the mixture at 0° C. and the resulting mixture was stirred at r.t. for 6 h under N₂. The mixture was concentrated and the residue was purified by flash column chromatography (MeOH:DCM=0 to 10%) to give the title compound as a yellow solid.

### Step 7: N-(2-Fluoro-3-(5-(2-(((1r,4r)-4-(methylsulfonyl)cyclohexyl)-amino)pyrimidin-4-yl)-2-(3-(trifluoromethyl)bicyclo[1.1.1]pentan-1-yl)thiazol-4-yl)phenyl)-indoline-1-sulfonamide

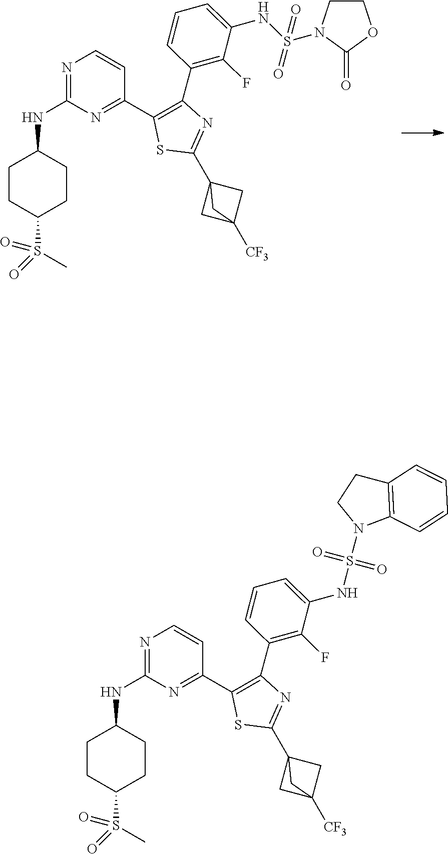 [SMILES: CS(=O)(=O)[C@H]1CC[C@H](Nc2nccc(-c3sc(C45CC(C(F)(F)F)(C4)C5)nc3-c3cccc(NS(=O)(=O)N4CCOC4=O)c3F)n2)CC1 ; CS(=O)(=O)[C@H]1CC[C@H](Nc2nccc(-c3sc(C45CC(C(F)(F)F)(C4)C5)nc3-c3cccc(NS(=O)(=O)N4CCc5ccccc54)c3F)n2)CC1]

A mixture of N-(2-fluoro-3-(S-(2-(((1r,4r)-4-(methylsulfonyl)cyclohexyl)-amino)-pyrimidin-4-yl)-2-(3-(trifluoromethyl)bicyclo[1.1.1]pentan-1-yl)thiazol-4-yl)phenyl)-2-oxooxazolidine-3-sulfonamide (60 mg, 0.08 mmol, 1.00 eq), indoline (30 mg, 0.25 mmol, 3.00 eq), and TEA (50 mg, 0.49 mmol, 6.00 eq) in MeCN (2.0 mL) was stirred at 100° C. for 7 h under microwave irradiation. The mixture was concentrated and the residue was purified by prep-TLC (EA:PE=1:1 to 2:1) to give the title compound as a white solid. MS (ES, m/z): [M+1]⁺=763.3.

Proceeding analogously as described in Example 68, the following compounds were prepared.

| | | | LCMS |
|---|---|---|---|
| | | | (ES, m/z): |
| Ex# | Compound | Synthesis | [M + H]⁺ |
| 69 | 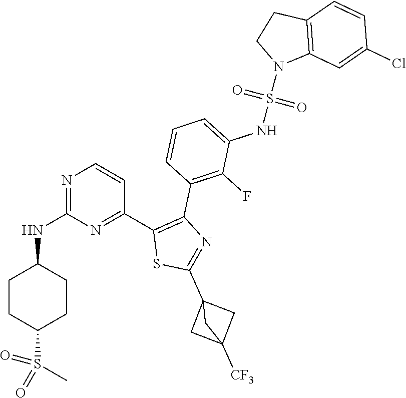 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(Cl)cc54)c3F)n2)CC1] | Indoline replaced by 6-chloroindoline in Step 7 | 796.9 |
| 70 | 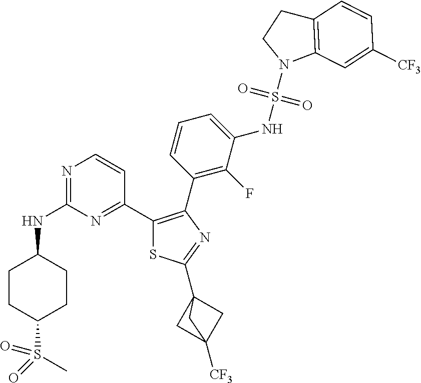 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(C(F)(F)F)cc54)c3F)n2)CC1] | Indoline replaced by 6- (trifluoromethyl)-indoline in Step 7 | 830.9 |
| 71 | 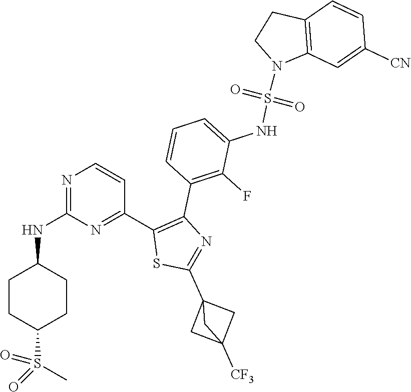 [SMILES: CS(=O)(=O)[C@H]1CC[C@H](Nc2nccc(-c3sc(C45CC(C(F)(F)F)(C4)C5)nc3-c3cccc(NS(=O)(=O)N4CCc5ccc(C#N)cc54)c3F)n2)CC1] | Indoline replaced by indoline-6- carbonitrile in Step 7 | 788.2 |
| 72 | 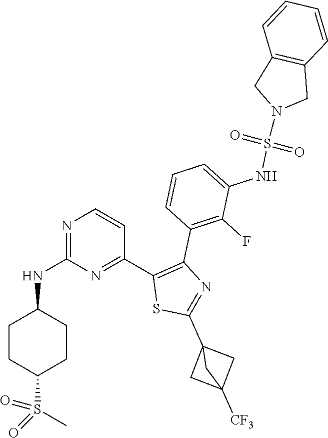 [SMILES: CS(=O)(=O)[C@H]1CC[C@H](Nc2nccc(-c3sc(C45CC(C(F)(F)F)(C4)C5)nc3-c3cccc(NS(=O)(=O)N4Cc5ccccc5C4)c3F)n2)CC1] | Indoline replaced by isoindoline in Step 7 | 763.4 |
| 73 | 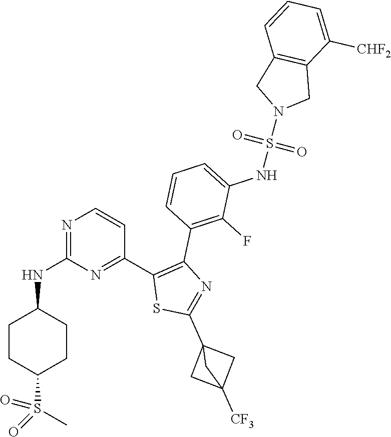 [SMILES: CS(=O)(=O)[C@H]1CC[C@H](Nc2nccc(-c3sc(C45CC(C(F)(F)F)(C4)C5)nc3-c3cccc(NS(=O)(=O)N4Cc5cccc(C(F)F)c5C4)c3F)n2)CC1] | Indoline replaced by Intermediate 8 in Step 7 | 813.2 |
| 74 | 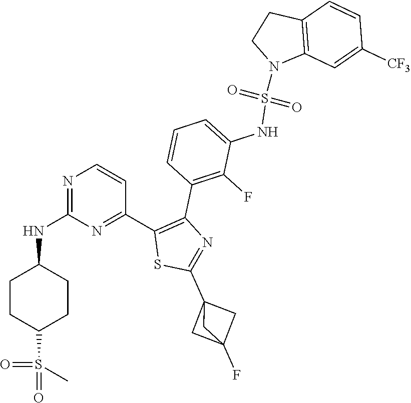 | 1. 3-(trifluoromethyl)bicyclo- [1.1.1]pentane-1-carboxylic acid replaced by 3-fluorobicyclo- [1.1.1]pentane-1-carboxylic acid in Step 1 2. Indoline replaced by 6-(trifluoro- methyl)indoline in Step 7 | 781.2 |
| 75 | 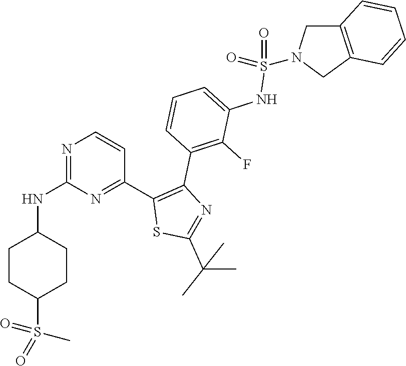 [SMILES: CC(C)(C)c1nc(-c2cccc(NS(=O)(=O)N3Cc4ccccc4C3)c2F)c(-c2ccnc(NC3CCC(S(C)(=O)=O)CC3)n2)s1] | 1. 3-(trifluoromethyl)bicyclo- [1.1.1]pentane-1-carbothioamide replaced by 2,2-dimethylpropane- thioamide in Step 3 2. Indoline replaced by isoindoline in Step 7 | 685.1 |
| 76 | 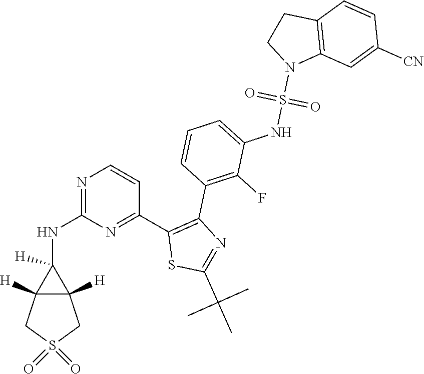 [SMILES: CC(C)(C)c1nc(-c2cccc(NS(=O)(=O)N3CCc4ccc(C#N)cc43)c2F)c(-c2ccnc(N[C@H]3[C@@H]4CS(=O)(=O)C[C@@H]43)n2)s1] | 1. 3-(trifluoromethyl)bicyclo- [1.1.1]pentane-1-carbothioamide replaced by 2,2-dimethylpropane- thioamide in Step 3 2. Intermediate 5 replaced by Intermediate 3 in Step 4 3. Indoline replaced by indoline-6- carbonitrile in Step 7 | 680.1 |

### Example 77

### Synthesis of N-(3-(2-(3-(3,3-difluorocyclobutyl)-3,8-diazabicyclo-[3.2.1]octan-8-yl)-5-(2-((2,2-dioxido-2-thiaspiro[3.3]heptan-6-yl)amino)pyrimidin-4-yl)thiazol-4-yl)-2-fluorophenyl)-4-(difluoromethyl)isoindoline-2-sulfonamide

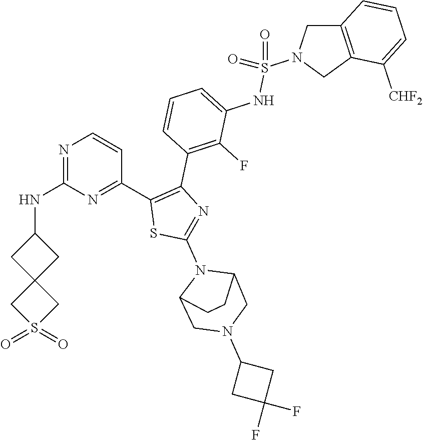 [SMILES: O=S1(=O)CC2(CC(Nc3nccc(-c4sc(N5C6CCC5CN(C5CC(F)(F)C5)C6)nc4-c4cccc(NS(=O)(=O)N5Cc6cccc(C(F)F)c6C5)c4F)n3)C2)C1]

### Step 1: N-(3-(2-Amino-5-(2-chloropyrimidin-4-yl)thiazol-4-yl)-2-fluorophenyl)acetamide

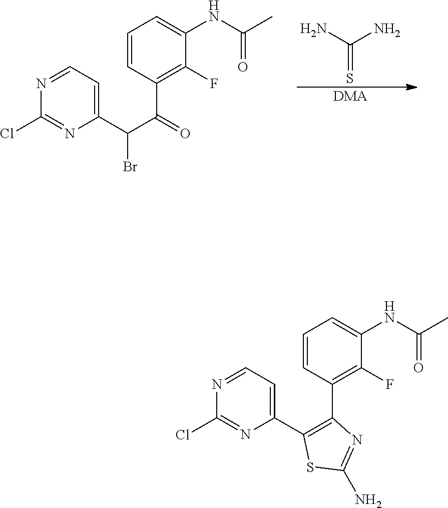

A mixture of N-(3-(2-bromo-2-(2-chloropyrimidin-4-yl)acetyl)-2-fluorophenyl)-acetamide (6.32 g, 16.40 mmol, 1.00 eq) and thiourea (1.25 g, 16.40 mmol, 1.00 eq) in DMA (70.0 mL) was stirred at 65° C. for 3 h under N₂. The mixture was poured into water and extracted with EtOAc. The combined organic layers were washed with water, brine, dried over Na₂SO₄, filtered and concentrated to give the title compound as a yellow solid.

### Step 2: N-(3-(2-Amino-5-(2-((2,2-dioxido-2-thiaspiro[3.3]heptan-6-yl)-amino)pyrimidin-4-yl)thiazol-4-yl)-2-fluorophenyl)acetamide

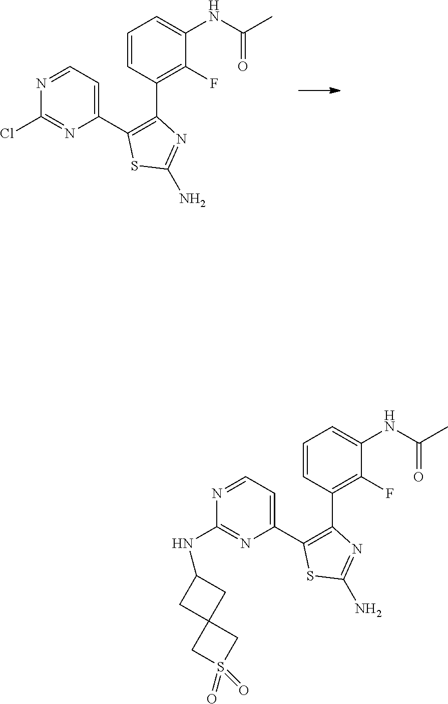 [SMILES: CC(=O)Nc1cccc(-c2nc(N)sc2-c2ccnc(Cl)n2)c1F ; CC(=O)Nc1cccc(-c2nc(N)sc2-c2ccnc(NC3CC4(C3)CS(=O)(=O)C4)n2)c1F]

A mixture of N-(3-(2-amino-5-(2-chloropyrimidin-4-yl)thiazol-4-yl)-2-fluorophenyl)-acetamide (5.05 g, 13.90 mmol, 1.00 eq), 2-thiaspiro[3.3]heptan-6-amine hydrochloride (Intermediate 6:3.30 g, 16.70 mmol, 1.50 eq), and DIEA (8.90 g, 69.60 mmol, 5.00 eq) in n-BuOH (100.0 mL) was stirred at 130° C. overnight under N₂. The mixture was poured into water and extracted with EtOAc. The combined organic layers were washed with water, brine, dried over Na₂SO₄, filtered, and concentrated. The residue was purified by column chromatography on silica gel (PE:EA=1:1 to DCM:MeOH=20:1) to give the title compound as a yellow solid.

### Step 3: N-(3-(2-Bromo-5-(2-((2,2-dioxido-2-thiaspiro[3.3]heptan-6-yl)amino)-pyrimidin-4-yl)-thiazol-4-yl)-2-fluorophenyl)acetamide

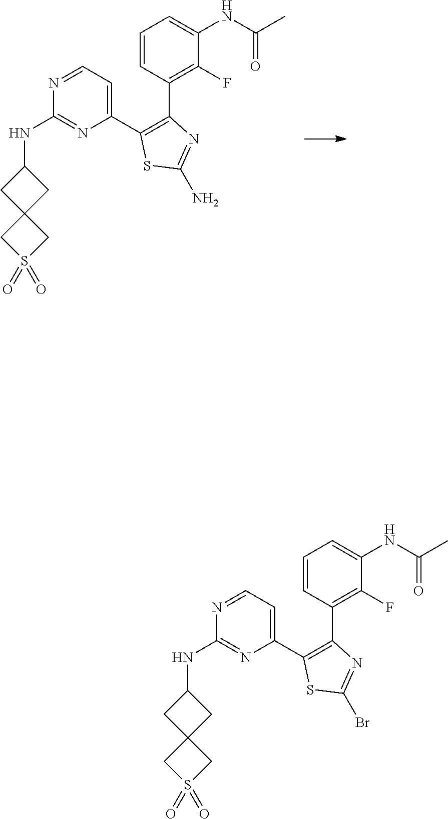 [SMILES: CC(=O)Nc1cccc(-c2nc(N)sc2-c2ccnc(NC3CC4(C3)CS(=O)(=O)C4)n2)c1F ; CC(=O)Nc1cccc(-c2nc(Br)sc2-c2ccnc(NC3CC4(C3)CS(=O)(=O)C4)n2)c1F]

tert-Butyl nitrite (1.15 g, 11.20 mmol, 1.50 eq) was added to a solution of N-(3-(2-amino-5-(2-((2,2-dioxido-2-thiaspiro[3.3]heptan-6-yl)amino)pyrimidin-4-yl)thiazol-4-yl)-2-fluoro-phenyl)acetamide (3.63 g, 7.40 mmol, 1.00 eq) and CuBr₂ (2.16 g, 9.70 mmol, 1.30 eq) in MeCN (50.0 mL) at 0° C. and the mixture was stirred at r.t. for 3 h under N₂. The mixture was poured into water and extracted with EtOAc. The combined organic layers were washed with water, brine, dried over Na₂SO₄, filtered, and concentrated. The residue was purified by column chromatography (PE:EA=1:1 to DCM:MeOH=20:1) to give the title compound as a yellow solid.

### Step 4: tert-Butyl 8-(4-(3-acetamido-2-fluorophenyl)-5-(2-((2,2-dioxido-2-thiaspiro[3.3]heptan-6-yl)amino)pyrimidin-4-yl)thiazol-2-yl)-3,8-diazabicyclo[3.2.1]octane-3-carboxylate

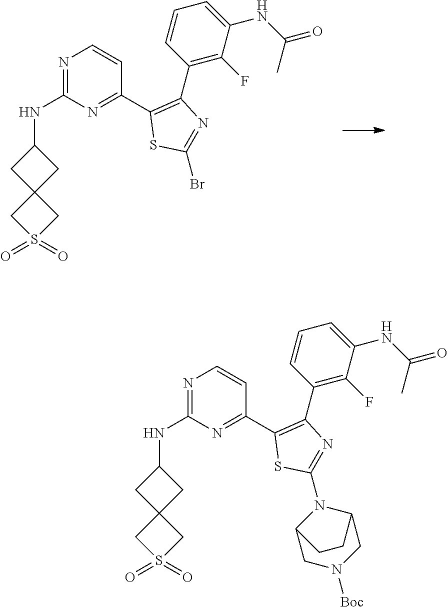 [SMILES: CC(=O)Nc1cccc(-c2nc(Br)sc2-c2ccnc(NC3CC4(C3)CS(=O)(=O)C4)n2)c1F ; CC(=O)Nc1cccc(-c2nc(N3C4CCC3CN(C(=O)OC(C)(C)C)C4)sc2-c2ccnc(NC3CC4(C3)CS(=O)(=O)C4)n2)c1F]

A mixture of N-(3-(2-bromo-5-(2-((2,2-dioxido-2-thiaspiro[3.3]heptan-6-yl)amino)-pyrimidin-4-yl)thiazol-4-yl)-2-fluorophenyl)acetamide (800 mg, 1.45 mmol, 1.00 eq.), tert-butyl 3,8-diazabicyclo[3.2.1]octane-3-carboxylate (400 mg, 1.88 mmol, 1.30 eq.) and TEA (439 mg, 4.38 mmol, 3.00 eq.) in DMA (10.0 mL) was stirred at 120° C. overnight. The mixture was diluted with water and extracted with EtOAc. The combined organic layers were washed with water, brine, dried over Na₂SO₄, filtered, and concentrated. The residue was purified by silica flash column PE/EtOAc (1:2) to give the title compound as a yellow solid.

### Step 5: tert-Butyl 8-(4-(3-amino-2-fluorophenyl)-5-(2-((2,2-dioxido-2-thiaspiro[3.3]heptan-6-yl)-amino)pyrimidin-4-yl)thiazol-2-yl)-3,8-diazabicyclo[3.2.1]octane-3-carboxylate

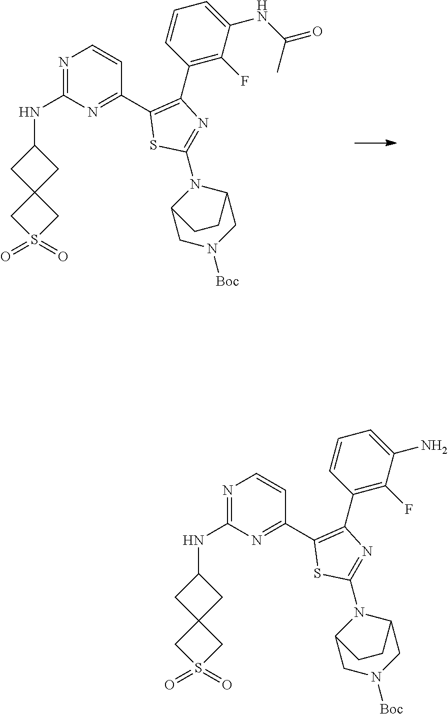 [SMILES: CC(=O)Nc1cccc(-c2nc(N3C4CCC3CN(C(=O)OC(C)(C)C)C4)sc2-c2ccnc(NC3CC4(C3)CS(=O)(=O)C4)n2)c1F ; CC(C)(C)OC(=O)N1CC2CCC(C1)N2c1nc(-c2cccc(N)c2F)c(-c2ccnc(NC3CC4(C3)CS(=O)(=O)C4)n2)s1]

NaOH (105 mg, 2.64 mmol, 2.00 eq.) was added to tert-butyl 8-(4-(3-acetamido-2-fluorophenyl)-5-(2-((2,2-dioxido-2-thiaspiro[3.3]heptan-6-yl)amino)pyrimidin-4-yl)thiazol-2-yl)-3,8-diazabicyclo[3.2.1]octane-3-carboxylate (900 mg, 1.32 mmol, 1.00 eq.) in EtOH (10.0 mL) and the mixture was stirred at 80° C. overnight. The reaction mixture was diluted with water and extracted with EtOAc. The combined organic layers were washed with brine, dried over Na₂SO₄, filtered, and concentrated to give the title compound as a yellow solid.

### Step 6: tert-Butyl 8-(5-(2-((2,2-dioxido-2-thiaspiro[3.3]heptan-6-yl)amino)pyrimidin-4-yl)-4~ (2-fluoro-3-(2-oxooxazolidine-3-sulfonamido)phenyl)thiazol-2-yl)-3,8-diazabicyclo[3.2.1]octane-3-carboxylate

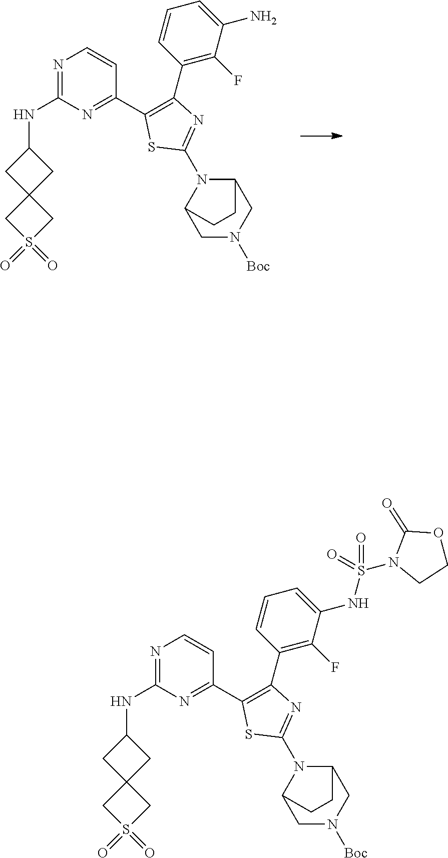 [SMILES: CC(C)(C)OC(=O)N1CC2CCC(C1)N2c1nc(-c2cccc(N)c2F)c(-c2ccnc(NC3CC4(C3)CS(=O)(=O)C4)n2)s1 ; CC(C)(C)OC(=O)N1CC2CCC(C1)N2c1nc(-c2cccc(NS(=O)(=O)N3CCOC3=O)c2F)c(-c2ccnc(NC3CC4(C3)CS(=O)(=O)C4)n2)s1]

2-Bromoethanol (215 mg, 1.75 mmol, 2.00 eq.) was added to a mixture of sulfurisocyanatidic chloride (245 mg, 1.75 mmol, 2.00 eq.) in DCM (12.0 mL) at 0° C. under N₂ and the mixture was stirred for 20 min. The mixture was slowly added to a solution of tert-butyl 8-(4-(3-amino-2-fluorophenyl)-5-(2-((2,2-dioxido-2-thiaspiro[3.3]heptan-6-yl)amino)pyrimidin-4-yl)thiazol-2-yl)-3,8-diazabicyclo[3.2.1]octane-3-carboxylate (560 mg, 0.87 mmol, 1.00 eq.), and TEA (441 mg, 4.37 mmol, 5.00 eq.) in DCM (30.0 mL) and the resulting mixture was stirred at rt for 6 h. The mixture was concentrated and purified by silica flash column DCM/MeOH (20:1) to give the title compound as a yellow solid.

### Step 7: tert-Butyl 8-(4-(3-(4-(difluoromethyl)isoindoline-2-sulfonamido)-2-fluorophenyl)-5-(2-((2,2-dioxido-2-thiaspiro[3.3]heptan-6-yl)amino)pyrimidin-4-yl)thiazol-2-yl)-3,8-diazabicyclo[3.2.1]octane-3-carboxylate

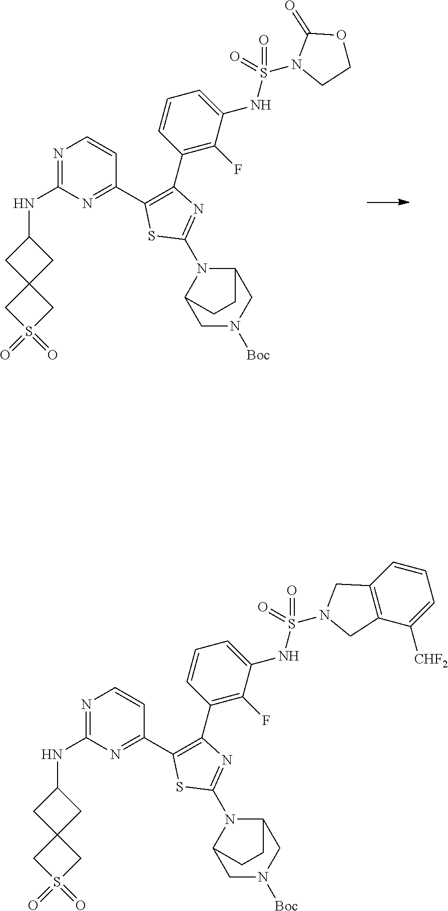 [SMILES: CC(C)(C)OC(=O)N1CC2CCC(C1)N2c1nc(-c2cccc(NS(=O)(=O)N3CCOC3=O)c2F)c(-c2ccnc(NC3CC4(C3)CS(=O)(=O)C4)n2)s1 ; CC(C)(C)OC(=O)N1CC2CCC(C1)N2c1nc(-c2cccc(NS(=O)(=O)N3Cc4cccc(C(F)F)c4C3)c2F)c(-c2ccnc(NC3CC4(C3)CS(=O)(=O)C4)n2)s1]

A mixture of tert-butyl 8-(5-(2-((2,2-dioxido-2-thiaspiro[3.3]heptan-6-yl)amino)pyrimidin-4-yl)-4-(2-fluoro-3-(2-oxooxazolidine-3-sulfonamido)phenyl)thiazol-2-yl)-3,8-diazabicyclo-[3.2.1]octane-3-carboxylate (100 mg, 0.13 mmol, 1.00 eq.), 4-(difluoromethyl)-isoindoline (Intermediate 8; 32 mg, 0.19 mmol, 1.50 eq.) and TEA (38 mg, 0.38 mmol, 3.00 eq.) in MeCN (2.0 mL) was heated at 130° C. under microwave irradiation for 3 h. The mixture was concentrated and the residue was purified by silica flash column DCM/MeOH (20:1) to give the title compound as a yellow solid.

### Step 8: N-(3-(2-(3,8-Diazabicyclo[3.2.1]octan-8-yl)-5-(2-((2,2-dioxido-2-thiaspiro[3.3]heptan-6-yl)amino)pyrimidin-4-yl)thiazol-4-yl)-2-fluorophenyl)-4-(difluoromethyl)isoindoline-2-sulfonamide

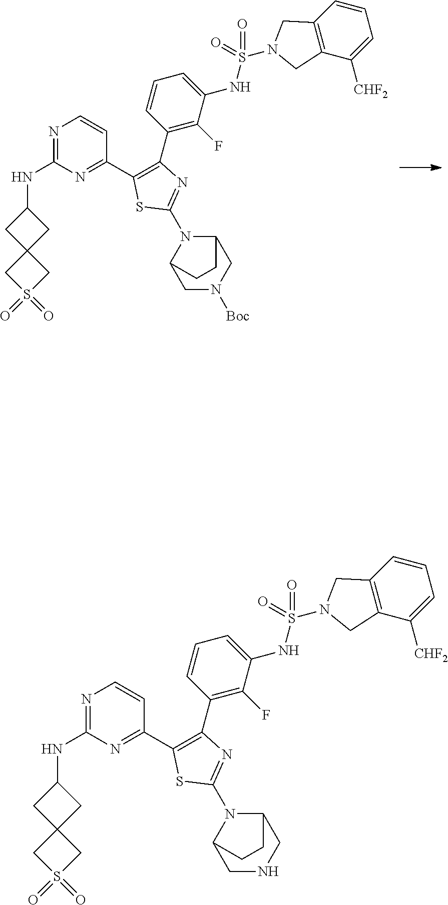 [SMILES: CC(C)(C)OC(=O)N1CC2CCC(C1)N2c1nc(-c2cccc(NS(=O)(=O)N3Cc4cccc(C(F)F)c4C3)c2F)c(-c2ccnc(NC3CC4(C3)CS(=O)(=O)C4)n2)s1 ; O=S1(=O)CC2(CC(Nc3nccc(-c4sc(N5C6CCC5CNC6)nc4-c4cccc(NS(=O)(=O)N5Cc6cccc(C(F)F)c6C5)c4F)n3)C2)C1]

TFA (1.0 mL) was added to a solution of tert-butyl 8-(4-(3-(4-(difluoromethyl)isoindoline-2-sulfonamido)-2-fluorophenyl)-5-(2-((2,2-dioxido-2-thiaspiro[3.3]heptan-6-yl)amino)pyrimidin-4-yl)thiazol-2-yl)-3,8-diazabicyclo[3.2.1]octane-3-carboxylate (80 mg, 0.92 mmol, 1.00 eq.) in DCM (4.0 mL) and the mixture was stirred at rt for 3 h. The solution was concentrated to give the crude product as a yellow oil.

### Step 9: N-(3-(2-(3-(3,3-Difluorocyclobutyl)-3,8-diazabicyclo[3.2.1]octan-8-yl)-5-(2-((2,2-dioxido-2-thiaspiro[3.3]heptan-6-yl)amino)pyrimidin-4-yl)thiazol-4-yl)-2-fluorophenyl)-4-(difluoromethyl)isoindoline-2-sulfonamide

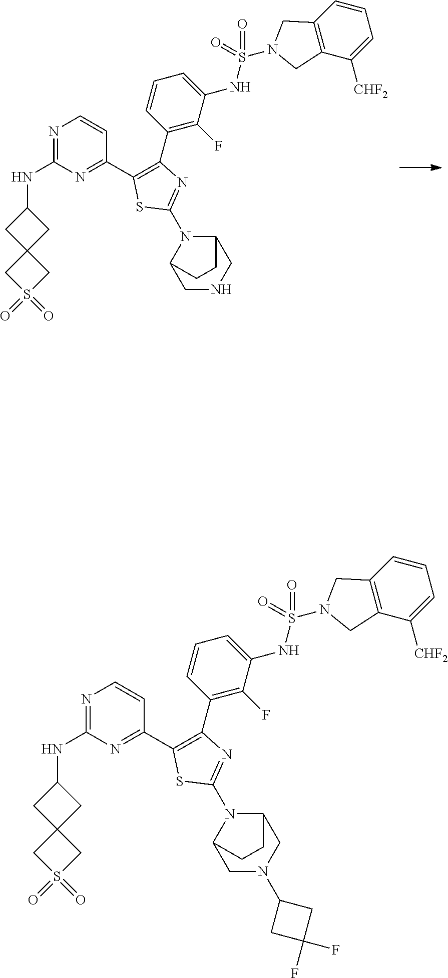 [SMILES: O=S1(=O)CC2(CC(Nc3nccc(-c4sc(N5C6CCC5CNC6)nc4-c4cccc(NS(=O)(=O)N5Cc6cccc(C(F)F)c6C5)c4F)n3)C2)C1 ; O=S1(=O)CC2(CC(Nc3nccc(-c4sc(N5C6CCC5CN(C5CC(F)(F)C5)C6)nc4-c4cccc(NS(=O)(=O)N5Cc6cccc(C(F)F)c6C5)c4F)n3)C2)C1]

A mixture of N-(3-(2-(3,8-diazabicyclo[3.2.1]octan-8-yl)-5-(2-((2,2-dioxido-2-thiaspiro[3.3]heptan-6-yl)amino)pyrimidin-4-yl)thiazol-4-yl)-2-fluorophenyl)-4-(difluoro-methyl) isoindoline-2-sulfonamide (88 mg, 0.11 mmol, 1.00 eq.), AcOH (1 drops) and 3,3-difluorocyclobutanone (60 mg, 0.57 mmol, 5.00 eq.) in MeOH/THF (2.0 mL, 1:1) was stirred at rt for 20 min. NaBH₃CN (36 mg, 0.57 mmol, 5.00 eq.) was added and the mixture was stirred at rt overnight. The mixture was diluted with water and extracted with DCM. The combined organic layers were dried and concentrated. The crude product was purified by prep-HPLC to give the title compound as a yellow solid. MS (ES, m/z): [M+1]⁺=863.2

### BIOLOGICAL EXAMPLES

### Biological Example 1: Phospho-RB Measurement in OVCAR3 Cells

The ability of compounds of Formula (I) to inhibit CDK2 was determined in OVCAR3 cells by measuring inhibition of phosphorylation of RB protein at S780 and S807/811. Phosphorylation of RB protein at S807/811 were measured using HTRF phospho-RB cellular kits (Cat #64RBS807PEG) from Cisbio.

On Day 1, OVCAR3 cells were seeded into 96-well tissue-culture treated plates at 20,000 cells/well in 200 μL and incubated overnight at 37° C. in CO₂ atmosphere. On Day 2, the cells were treated with test compounds at concentrations from 0.3 to 10,000 nM using HP D300 digital dispenser. Twenty-four hours after compound treatment, cell culture media was removed by flicking the plate and tapping the plate against clean paper towel. Immediately 30 μL 1× lysis buffer was supplemented from the kit and the plate was incubated at room temperature on shaker for 30 min. After homogenization by pipetting up and down, 8 μL cell lysate from 96-well cell culture plate was transferred to 384-well small volume white detection plate. 2 μL premixed detection solution was added and the plate was covered with sealer. To prepare the detection solution, d2 conjugated-phospho-RB antibody and Eu-cryptate conjugated phosphor-RB antibody were diluted into detection buffer following manufacturer's instruction.

Detection plates were incubated for 4 h at room temperature and read on ClarioStar (BMG Labtech) in TR-FRET mode (665 nM and 620 nM). The TR-FRET ratio (665 nM/620 nM) was plotted against the compound concentration and normalized to DMSO controls. Half maximal inhibition concentration (IC₅₀) values are calculated with a four-parameter logistic fit using GraphPad Prism (version 8; La Jolla, CA). CDK2 IC₅₀ data for compounds in Compound Table 1 above are provided in Table 2 below.

TABLE 2

| Cpd # (Cpd | IC₅₀ |
|---|---|
| Table 1 above) | (nM) |
| 1 | 108.5 |
| 2 | 75.5 |
| 3 | 48.6 |
| 4 | 138.5 |
| 5 | 32.7 |
| 6 | 108.8 |
| 7 | 9.2 |
| 8 | 15.2 |
| 9 | 104.1 |
| 10 | 73.0 |
| 11 | 81.0 |
| 12 | 47.8 |
| 13 | 57.7 |
| 14 | 123.6 |
| 15 | 72.5 |
| 16 | 62.4 |
| 17 | 15.0 |
| 18 | 24.8 |
| 19 | 120.4 |
| 20 | 208.3 |
| 21 | 55.7 |
| 22 | 476.9 |
| 23 | 136.3 |
| 24 | 102.5 |
| 25 | 75.1 |
| 26 | 160.3 |
| 27 | 161.4 |
| 28 | 71.9 |
| 29 | 70.8 |
| 30 | 28.5 |
| 31 | 30.2 |
| 32 | 23.1 |
| 33 | 153.2 |
| 34 | 116.2 |
| 35 | 1591 |
| 36 | 195.3 |
| 37 | 74.5 |
| 38 | 263 |
| 39 | 752.1 |
| 40 | 191.8 |
| 41 | 222.2 |
| 42 | 278 |
| 43 | 27.1 |
| 44 | 56.0 |
| 45 | 313.6 |
| 46 | 476.1 |
| 47 | 342.9 |
| 48 | 139.1 |
| 49 | 136.9 |
| 50 | 228.1 |
| 51 | 335.6 |
| 52 | 160 |
| 53 | 261.7 |
| 54 | 382.3 |
| 55 | 525.4 |
| 56 | 269.95 |
| 57 | 494.3 |
| 58 | 284.9 |
| 59 | 53.3 |
| 60 | NA |
| 61 | NA |
| 62 | 3175 |
| 63 | 678.7 |
| 64 | 735 |
| 65 | 588.45 |
| 66 | >5000 |
| 67 | 8258 |
| 68 | 5488 |
| 69 | 88.9 |
| 70 | 94 |
| 71 | 76 |
| 72 | 185 |
| 73 | 58 |
| 74 | 180.45 |
| 75 | 65 |
| 76 | 65 |
| 77 | 367.2 |
| 78 | 65.62 |
| 79 | 220 |
| 80 | 171.57 |
| 81 | 3730 |
| 82 | 850 |
| 83 | 1467 |
| 84 | 1920 |
| 85 | 394.5 |
| 86 | 484 |
| 87 | 324.95 |
| 88 | >10 μm |
| 89 | 4811 |
| 90 | >10 μm |
| 91 | 116.7 |
| 92 | 305 |
| 93 | 3025 |
| 94 | 5940 |
| 95 | 609.8 |
| 96 | 454 |
| 97 | 284.6 |
| 98 | 510.93 |
| 99 | 201.8 |
| 100 | 1244 |
| 101 | 1829 |
| 102 | 280.7 |
| 103 | 142.3 |
| 104 | 82 |
| 105 | 215 |
| 106 | 262.6 |
| 107 | 919 |
| 108 | 2072 |
| 109 | 210.1 |
| 110 | 1030 |
| 111 | 908 |
| 112 | 1211 |
| 113 | 120.42 |
| 114 | 275.8 |
| 115 | 635.7 |
| 116 | 389.7 |
| 117 | 330.7 |
| 118 | 66.9 |
| 119 | 396.8 |
| 120 | 86.9 |
| 121 | 264.9 |
| 122 | 54.0 |
| 123 | 75.19 |
| 124 | 150.8 |
| 125 | 203.6 |
| 126 | 366 |
| 127 | 208.2 |
| 128 | 162.2 |
| 129 | 327.7 |
| 130 | 268.05 |
| 131 | 160.03 |
| 132 | 309.8 |
| 133 | 331 |
| 134 | 589 |
| 135 | 474.4 |
| 136 | 1487 |
| 137 | 285.6 |
| 138 | 396.1 |
| 139 | 290.85 |
| 140 | 621.6 |
| 141 | 290.8 |
| 142 | 340.7 |
| 143 | 135.5 |
| 144 | 227 |
| 145 | 388.5 |
| 146 | 3007 |
| 147 | 458.4 |
| 148 | 210.4 |
| 149 | 487.6 |
| 150 | 1842 |
| 151 | 355 |
| 152 | 397.4 |
| 153 | 2820 |
| 154 | 685.1 |
| 155 | 1057 |
| 156 | 90.3 |
| 157 | 191.2 |
| 158 | 99.6 |
| 159 | 4081 |
| 160 | 4039 |
| 161 | 1799 |
| 162 | 2125 |
| 163 | 2431 |
| 164 | 2669 |
| 165 | 1398 |
| 166 | 276.6 |
| 167 | 409.6 |
| 168 | 1594 |
| 169 | 2247 |
| 170 | 62 |
| 171 | 2122 |
| 172 | 3165 |
| 173 | 385.2 |
| 174 | 853.4 |
| 175 | 942.6 |
| 176 | 1919 |
| 177 | 1022 |
| 178 | 203 |
| 179 | 241 |
| 180 | 2817 |
| 181 | 1885 |
| 182 | 1192 |
| 183 | 133.4 |
| 184 | 1542 |
| 185 | 55.0 |
| 186 | 1181 |
| 187 | 194.3 |
| 188 | 1657 |
| 189 | 1548 |
| 190 | 258.9 |
| 191 | 8619 |
| 192 | 88.23 |
| 193 | 125.1 |
| 194 | 1852 |
| 195 | 320 |
| 196 | 3008 |
| 197 | 1075 |
| 198 | 666.7 |
| 199 | >3845 |
| 200 | 247.3 |
| 201 | 76.33 |
| 202 | 86.16 |
| 203 | 930.5 |
| 204 | 69.17 |
| 205 | 40.4 |
| 206 | 619.1 |
| 207 | 198.8 |
| 208 | 3691 |
| 209 | 349.5 |
| 210 | 505.4 |
| 211 | 613.2 |
| 212 | 661.7 |
| 213 | 3304 |
| 214 | 534.7 |
| 215 | 601.2 |
| 216 | 65.4 |
| 217 | 934.2 |
| 218 | 1301 |
| 219 | 582.8 |
| 220 | 802.3 |
| 221 | 35.5 |
| 222 | 152.5 |
| 223 | 89 |
| 224 | 300.1 |
| 225 | 455.7 |
| 226 | 527.1 |
| 227 | 516.8 |
| 228 | 1514 |
| 229 | 145 |
| 230 | 354 |
| 231 | 77.4 |
| 232 | 938.9 |
| 233 | 764.5 |
| 234 | 1198 |
| 235 | 3394 |
| 236 | 476.6 |
| 237 | 297.4 |
| 238 | 295 |
| 239 | 4689 |
| 240 | 38.2 |
| 241 | 520.8 |
| 242 | 302.2 |
| 243 | 1202 |
| 244 | 1454 |
| 245 | 337.6 |
| 246 | 334.05 |
| 247 | 117 |
| 248 | 405.2 |
| 249 | 684.3 |
| 250 | 575.5 |
| 251 | 1532 |
| 252 | 3311 |
| 253 | 133.1 |
| 254 | 317 |
| 255 | 682.6 |
| 256 | 3078 |
| 257 | 2965 |
| 258 | 434.8 |
| 259 | 2041 |
| 260 | 661.7 |
| 261 | 694.5 |
| 262 | 31.2 |
| 263 | 118.3 |
| 264 | 1830 |
| 265 | 5269 |
| 266 | 8472 |
| 267 | 9414 |
| 268 | 8308 |
| 268 | 70.6 |
| 270 | 3429 |
| 271 | 8148 |
| 272 | 349.5 |
| 273 | 3277 |
| 274 | 130.46 |
| 275 | 338.8 |
| 276 | 192.2 |
| 277 | 111.1 |
| 278 | 103.1 |
| 279 | 408.95 |
| 280 | 285.4 |
| 281 | 739.33 |
| 282 | 357.45 |
| 283 | 200.3 |
| 284 | 383.9 |
| 285 | 3061 |
| 286 | 573.8 |
| 287 | 346.54 |
| 288 | 121.8 |
| 289 | 110.5 |
| 290 | 297.8 |
| 291 | 2753 |
| 292 | 29.87 |
| 293 | 372.3 |
| 294 | 42.86 |
| 295 | 536.2 |
| 296 | 1193 |
| 297 | 103.1 |
| 298 | 129.6 |
| 299 | 449.2 |
| 300 | 474.8 |

### Formulation Examples

The following are representative pharmaceutical formulations containing a compound of the present disclosure.

### Tablet Formulation

The following ingredients are mixed and pressed into single scored tablets as shown in Table 3.

TABLE 3

| | Quantity per |
|---|---|
| Ingredient | tablet (mg) |
| compound Formula (I) | 400 |
| cornstarch | 50 |
| croscarmellose sodium | 25 |
| lactose | 120 |
| magnesium stearate | 5 |

### Capsule Formulation

The following ingredients are mixed and loaded into a hard-shell gelatin capsule as shown in Table 4.

TABLE 4

| | Quantity per |
|---|---|
| Ingredient | capsule (mg) |
| compound Formula (I) | 200 |
| lactose spray dried | 148 |
| magnesium stearate | 2 |

### Injectable Formulation

A compound of the disclosure (e.g., a compound of Formula (I)) is added to a solution of 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound of the disclosure (e.g., a compound of Formula (I)) is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound of the disclosure (e.g., a compound of Formula (I)) is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound of the disclosure (e.g., a compound of Formula (I)) is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound of the disclosure (e.g., a compound of Formula (I)) is mixed with 30 mL of a 0.05M phosphate buffer solution (pH 4.4). The solution is placed in a nasal administrator designed to deliver 100 μl of spray for each application.

What is claimed:

1. A compound of Formula (I): 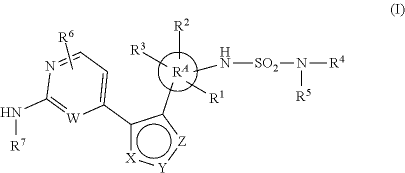
wherein:
W is N, CH, or C when W is attached to R⁶;
X and Z are independently CR⁸, NR^{8A}, N, O, or S; and Y is CR⁹, NR¹⁰, or N; provided that both of X and Z are not simultaneously O or S; and at least one of X, Y, and Z is not carbon;
R⁸ is hydrogen, alkyl, halo, hydroxyalkyl, or cyano;
R^{8A} is hydrogen or alkyl;
R⁹ and R¹⁰ are independently hydrogen, alkyl, haloalkyl, hydroxyalkyl, alkoxyalkyl, cyanoalkyl, alkylsulfonyl, alkylsulfonylalkyl, amino, alkylamino, dialkylamino, substituted amino, aminoalkyl, cycloalkyl, bicyclic cycloalkyl, bridged cycloalkyl, spiro cycloalkyl, heterocyclyl, bicyclic heterocyclyl, bridged heterocyclyl, spiro heterocyclyl, aryl, aralkyl, heteroaryl, or heteroaralkyl, wherein:
(A) cycloalkyl, bicyclic cycloalkyl, bridged cycloalkyl, and spiro cycloalkyl of R⁹ and R¹⁰ are substituted with one or two R^{a} independently selected from hydrogen, deuterium, alkyl, deuteroalkyl, cycloalkyl, halo, haloalkyl, hydroxy, hydroxyalkyl, cyano, amino, alkylamino, and dialkylamino;
(B) heterocyclyl, bicyclic heterocyclyl, bridged heterocyclyl, and spiro heterocyclyl of R⁹ and R¹⁰ are substituted with R^{b}, R^{c}, and R^{d} independently selected from hydrogen, alkyl, deuteroalkyl, cycloalkyl, bridged cycloalkyl, spiro cycloalkyl (wherein cycloalkyl, bridged cycloalkyl, and spiro cycloalkyl are substituted with one or two substituents independently selected from hydrogen, alkyl, halo, hydroxy, and cyano), alkoxy, halo, haloalkyl, haloalkoxy, alkoxycarbonyl, hydroxy, cyano, amino, alkylamino, dialkylamino, aryl, aralkyl, heterocyclyl, bridged heterocyclyl, spiro heterocyclyl, heteroaryl, or heteroaralkyl (wherein aryl, by itself or as part of aralkyl, heteroaryl, by itself or as part of heteroaralkyl, heterocyclyl, bridged heterocyclyl, and spiro heterocyclyl are substituted with R^{e}, R^{f}, and R^{g} independently selected from hydrogen, alkyl, cycloalkyl, halo, haloalkyl, alkoxy, haloalkoxy, hydroxy, cyano, amino, alkylamino, and dialkylamino); and
(C) aryl, by itself or as part of aralkyl, and heteroaryl, by itself or as part of heteroaralkyl, of R⁹ and R¹⁰ are substituted with R^{h}, R^{j}, and R^{k} independently selected from hydrogen, alkyl, deuteroalkyl, cycloalkyl (wherein cycloalkyl is optionally substituted with one or two substituents independently selected from alkyl, halo, hydroxy, and cyano), alkoxy, halo, haloalkyl, haloalkoxy, alkoxycarbonyl, hydroxy, cyano, amino, alkylamino, dialkylamino, aryl, aralkyl, heterocyclyl, bridged heterocyclyl, spiro heterocyclyl, heteroaryl, or heteroaralkyl (wherein aryl, by itself or as part of aralkyl, heteroaryl, by itself or as part of heteroaralkyl, heterocyclyl, bridged heterocyclyl, and spiro heterocyclyl are substituted with R^{m}, Rⁿ, and R^{o} independently selected from hydrogen, alkyl, cycloalkyl, halo, haloalkyl, alkoxy, haloalkoxy, hydroxy, cyano, amino, alkylamino, and dialkylamino);
ring R^{A} is phenyl or heteroaryl;
R¹, R², and R³ are independently hydrogen, deuterium, alkyl, halo, haloalkyl, haloalkoxy, alkoxy, hydroxy, hydroxyalkyl, alkoxyalkyl, aminoalkyl, or cyano;
R⁴ and R⁵ are independently hydrogen, alkyl, haloalkyl, alkoxyalkyl, hydroxalkyl, aminoalkyl, cycloalkyl, cycloalkylalkyl, fused cycloalkyl, aryl, aralkyl, heteroaryl, heteroaralkyl, heterocyclyl, heterocyclylalkyl, bicyclic heterocyclyl, bicyclic heterocyclylalkyl, bridged heterocyclyl, bridged heterocyclylalkyl, fused heterocyclyl, fused heterocyclylalkyl, spiro heterocyclyl, and spiro heterocyclylalkyl (wherein cycloalkyl, by itself or as part of cycloalkylalkyl, fused cycloalkyl, aryl, by itself or as part of aralkyl, heteroaryl, by itself or as part of heteroaralkyl, heterocyclyl, by itself or as part of heterocyclylalkyl, bicyclic heterocyclyl, by itself or as part of bicyclic heterocyclylalkyl, bridged heterocyclyl, by itself or as part of bridged heterocyclylalkyl, fused heterocyclyl, by itself or as part of fused heterocyclylalkyl, and spiro heterocyclyl, by itself or as part of spiro heterocyclylalkyl, are substituted with R^{p}, R^{q}, and R^{r} independently from hydrogen, alkyl, alkoxy, alkylsulfonyl, alkyloxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, aminoalkylamino, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, and heterocyclylalkyloxy (wherein aryl, by itself or as part of aralkyl and aryloxy, heteroaryl, by itself or as part of heteroaryloxy and heteroaralkyl, and heterocyclyl, by itself or as part of heterocyclylalkyl, heterocyclyloxy, and heterocyclylalkyloxy, are substituted with one, two, or three substituents independently selected from hydrogen, alkyl, alkoxy, hydroxy, halo, haloalkyl, haloalkoxy, and cyano)); or
R⁴ and R⁵ together with the nitrogen atom to which they are attached form cyclylaminyl, bicyclylaminyl, fused cyclylaminyl, bridged cyclylaminyl, fused bridged cyclylaminyl, spiroaminyl, or fused spiroaminyl, wherein each of the aforementioned rings is substituted with R^{s}, R^{t}, and R^{u} independently selected from hydrogen, deuterium, alkyl, alkoxy, alkylsulfonyl, alkoxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, aminoalkylamino, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, heterocyclylalkyloxy, and unsaturated heterocyclyl (wherein aryl, by itself or as part of aryloxy and aralkyl, heteroaryl, by itself or as part of heteroaryloxy and heteroaralkyl, heterocyclyl, by itself or as part of heterocyclylalkyl, heterocyclyloxy, and heterocyclylalkyloxy, and unsaturated heterocyclyl are substituted with one to three substituents independently selected from hydrogen, alkyl, alkoxy, hydroxy, halo, haloalkyl, haloalkoxy, and cyano);
R⁶ is hydrogen, alkyl, cycloalkyl, cyano, halo, or haloalkyl;
R⁷ is -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, -Q⁶-(alk⁷)ₙ₇-NR²¹SO₂R²², heterocyclyl, bicyclic heterocyclyl, fused heterocyclyl, spiro heterocyclyl, bridged heterocyclyl, cyclylsulfoximinyl, bicyclylsulfoximinyl, spiro sulfoximinyl, aryl, heteroaryl, or fused heteroaryl wherein:
each of Q, Q¹, Q², Q³, Q⁴, Q⁵, and Q⁶ is a bond, cycloalkyl, bridged cycloalkyl, spiro cycloalkyl, heterocyclyl^{A}, bicyclic heterocyclyl^{A}, bridged heterocyclyl^{A}, fused heterocyclyl^{A}, spiro heterocyclyl^{A}, aryl, or heteroaryl, wherein each of the aforementioned rings is substituted with R^{v} and R^{w} independently selected from hydrogen, deuterium, alkyl, halo, haloalkyl, haloalkoxy, alkoxy, and cyano;
each of n¹, n², n³, n⁴, n⁵, n⁶, and n⁷ is 0 or 1, provided that, when Q is a bond, then each of n¹, m², n³, n⁴, n⁵, n⁶, and n⁷ is 1;
each alk¹, alk², alk³, alk⁴, alk⁵, alk⁶, and alk⁷ is alkylene;
R¹², R¹⁹, and R²¹ are hydrogen or alkyl;
R¹¹, R¹³, R¹⁶, R²⁰, and R²² are independently selected from hydrogen, alkyl, fluoro, chloro, bromo, haloalkyl, hydroxyalkyl, alkoxyalkyl, aminoalkyl, cycloalkyl, cycloalkylalkyl, aryl, aralkyl, heteroaryl, heteroaralkyl, heterocyclyl, bicyclic heterocyclyl, bridged heterocyclyl, fused heterocyclyl, spiro heterocyclyl, heterocyclylalkyl, or —CR²³═CR²⁴R²⁵ [where R²³ is hydrogen, alkyl, or cyano, R²⁴ is hydrogen or alkyl, and R²⁵ is hydrogen, alkyl, halo, haloalkyl, alkoxyalkyl, hydroxyalkyl, amino, alkylamino, dialkylamino, or -(alkylene)-NR²⁶R²⁷ (where R²⁶ and R²⁷ are independently hydrogen, or alkyl), or heterocyclylalkyl], wherein cycloalkyl, by itself or as part of cycloalkylalkyl, aryl, by itself or as part of aralkyl, heteroaryl, by itself or as part of heteroaralkyl, heterocyclyl, by itself or as part of heterocyclylalkyl, bicyclic heterocyclyl, bridged heterocyclyl, fused heterocyclyl, and spiro heterocyclyl of R¹¹, R¹³, R¹⁶, R²⁰), and R²² and heterocyclyl of heterocyclylalkyl of R²⁵ are substituted with one to three substituents independently selected from hydrogen, alkyl, alkoxy, hydroxy, halo, haloalkyl, haloalkoxy, hydroxyalkyl, alkoxyalkyl, aminoalkyl, cyano, and heterocyclyl; provided R¹¹, R¹⁶, R²⁰, and R²² are not hydrogen;
R¹⁴, R¹⁵, R¹⁷, and R¹⁸ are independently selected from hydrogen, alkyl, haloalkyl, hydroxyalkyl, alkoxyalkyl, aminoalkyl, cycloalkyl, cycloalkylalkyl, aryl, aralkyl, heteroaryl, heteroaralkyl, heterocyclyl, or heterocyclylalkyl, wherein cycloalkyl, by itself or as part of cycloalkylalkyl, aryl, by itself or as part of aralkyl, heteroaryl, by itself or as part of heteroaralkyl, and heterocyclyl, by itself or as part heterocyclylalkyl, are substituted with one to three substituents independently selected from hydrogen, alkyl, alkoxy, hydroxy, halo, haloalkyl, haloalkoxy, and cyano; and
the heterocyclyl, bicyclic heterocyclyl, fused heterocyclyl, spiro heterocyclyl, bridged heterocyclyl, cyclylsulfoximinyl, spiro cyclylsulfoximinyl, aryl, heteroaryl, and fused heteroaryl of R⁷ are substituted with R^{x}, R^{y}, and R^{y1} independently selected from hydrogen, deuterium, alkyl, alkoxy, alkoxycarbonyl, hydroxy, acyl, halo, haloalkyl, haloalkoxy, amino, alkylamino, dialkylamino, substituted amino, alkoxycarbonylamino, alkoxycarbonylaminoalkyl, aminocarbonyl, aminocarbonylalkyl, cyano, hydroxyalkyl, hydroxyalkoxy, alkoxyalkyl, alkoxyalkyloxy, alkoxyalkyloxyalkyl, aminoalkyl, aminoalkoxy, aminoalkylamino, cyanoalkyl, cyanoalkoxy, cycloalkyl, cycloalkoxy, aryl, aryloxy, aralkyl, heteroaryl heteroaryloxy, heteroaralkyl, heterocyclyl, heterocyclylalkyl, heterocyclyloxy, heterocyclylalkyloxy (wherein aryl, by itself or as part of aralkyl and aryloxy, heteroaryl, by itself or as part, of heteroaryloxy and heteroaralkyl, and heterocyclyl, by itself or as part of, heterocyclylalkyl, heterocyclyloxy, and heterocyclylalkyloxy, are substituted with one to three substituents independently selected from hydrogen, alkyl, alkoxy, hydroxy, halo, haloalkyl, haloalkoxy, alkoxycarbonyloxy, alkoxyalkyl, alkoxyalkyloxyalkyl, cyano, and heterocyclyl); or
a pharmaceutically acceptable salt thereof, provided that the compound of Formula (I) is not:
N-(3-(2-isopropyl-S-(2-((2-(methylsulfonyl)ethyl)amino)pyrimidin-4-yl)thiazol-4-yl)-phenyl)morpholine-4-sulfonamide.

2. The compound of claim 1, or a pharmaceutically acceptable salt thereof, wherein: 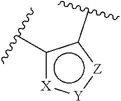
is

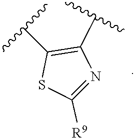

3. The compound of claim 1 or 2, or a pharmaceutically acceptable salt thereof, having a structure according to formula (Ia):

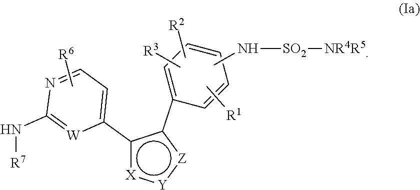

4. The compound of any one of claims 1 to 3, or a pharmaceutically acceptable salt thereof, having a structure according to formula (Id′):

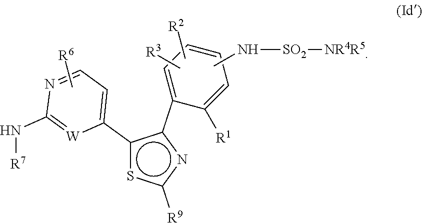

5. The compound of any one of claims 1 to 4, or a pharmaceutically acceptable salt thereof, wherein R¹, R², and R³ are independently hydrogen, deuterium, alkyl, halo, haloalkyl, haloalkoxy, alkoxy, hydroxy, or cyano.

6. The compound of any one of claims 1 to 5, or a pharmaceutically acceptable salt thereof, wherein R¹ is fluoro, R² is hydrogen, deuterium, fluoro, chloro, difluoromethyl, trifluoromethyl, difluoromethoxy, trifluoromethoxy, or cyano, and R³ is hydrogen.

7. The compound of any one of claims 1 to 6, or a pharmaceutically acceptable salt thereof, wherein R′ is fluoro and R² and R³ are hydrogen.

8. The compound of any one of claims 1 to 7, or a pharmaceutically acceptable salt thereof, wherein R⁴ and R⁵ together with the nitrogen atom to which they are attached form cyclylaminyl, bicyclylaminyl, fused cyclylaminyl, bridged cyclylaminyl, or spiroaminyl, wherein cyclylaminyl, bicyclylaminyl, fused cyclylaminyl, bridged cyclylaminyl, and spiroaminyl are substituted with R^{s}, R^{t}, and R^{u}.

9. The compound of any one of claims 1 to 8, or a pharmaceutically acceptable salt thereof, wherein R⁴ and R⁵ together with the nitrogen atom to which they are attached form fused cyclylaminyl substituted with R^{s}, R^{t}, and R^{u}.

10. The compound of any one of claims 1 to 9, or a pharmaceutically acceptable salt thereof, wherein fused cyclylaminyl formed by R⁴ and R⁵ together with the nitrogen atom to which they are attached has a structure according to formula (a): 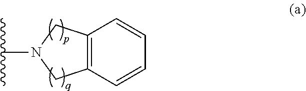
wherein:
p is 0, 1, or 2; and q is 0, 1, 2, 3, or 4; provided p+q is at least 2 and p+q is not more than 5; and when p or q is 3 or 4, then one of the —CH₂— of such p or q can be replaced NH, N (when attached to one of R^{s}, R^{t}, and R^{u}), O or S(O)ₙ; wherein n is 0, 1, or 2; and
the structure of formula (a) is substituted with R^{s}, R^{t}, and R^{u}.

11. The compound of any one of claims 1 to 9, or a pharmaceutically acceptable salt thereof, wherein the fused cyclylaminyl formed by R⁴ and R⁵ together with the nitrogen atom to which they are attached has a structure according to formula (b): 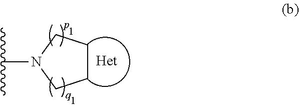
wherein:
p₁ is 0, 1, or 2; and q₁ is 0, 1, 2, 3, or 4; provided p+q is at least 2 and p+q is not more than 5; and when p or q is 3 or 4, then one of the —CH₂— of such p or q can be replaced by NH, N (when attached to one of R^{s}, R^{t}, and R^{u}), O or S(O)ₙ; wherein n is 0, 1, or 2;
Het is 5- or 6-membered heteroaryl; and
the structure of formula (b) is substituted with R^{s}, R^{t}, and R^{u}.

12. The compound of any one of claims 1 to 11, or a pharmaceutically acceptable salt thereof, wherein R⁴ and R⁵ together with the nitrogen atom to which they are attached form a ring selected form: 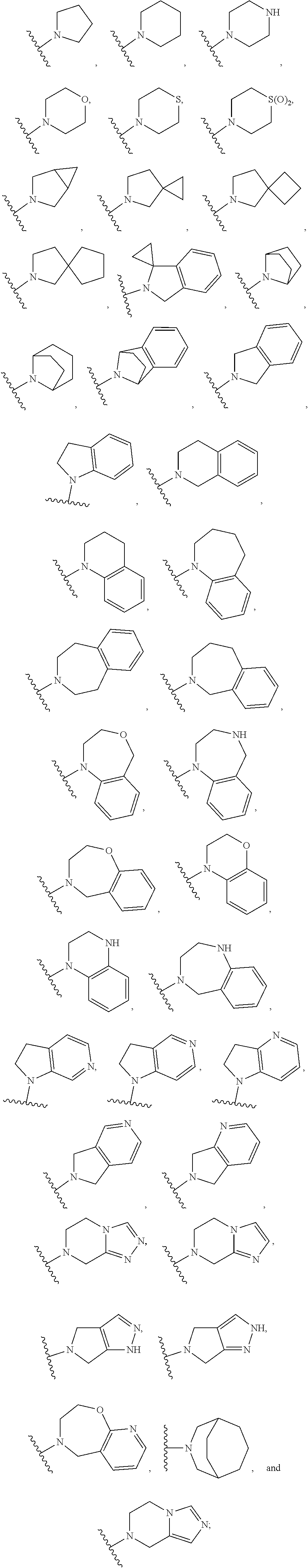
wherein each ring is substituted with R^{s}, R^{t}, and R^{u}.

13. The compound of any one of claims 1 to 12, or a pharmaceutically acceptable salt thereof, wherein R^{s} is hydrogen, deuterium, alkyl, alkoxy, alkylsulfonyl, hydroxy, acyl, halo, haloalkyl, haloalkoxy, amino, alkylamino, dialkylamino, substituted amino, alkoxycarbonylaminoalkyl, aminocarbonyl, aminocarbonylalkyl, cyano, hydroxyalkyl, hydroxyalkoxy, alkoxyalkyl, alkoxyalkyloxy, aminoalkyl, aminoalkoxy, cyanoalkyl, cyanoalkoxy, cycloalkyl, cycloalkoxy, aryl, aryloxy, aralkyl, heteroaryl, heteroaryloxy, heteroaralkyl, heterocyclyl, heterocyclylalkyl, heterocyclyloxy, or unsaturated heterocyclyl (wherein aryl, by itself or as part of aryloxy and aralkyl, heteroaryl, by itself or as part of heteroaryloxy and heteroaralkyl heterocyclyl, by itself or as part of heterocyclylalkyl and heterocyclyloxy and unsaturated heterocyclyl are substituted with one to three substituents independently selected from hydrogen, alkyl, alkoxy, hydroxy, halo, haloalkyl, haloalkoxy, and cyano), R^{t} is hydrogen, alkyl, alkoxy, alkylsulfonyl, hydroxy, acyl, halo, haloalkyl, haloalkoxy, amino, alkylamino, dialkylamino, and cyano, and R^{u} is hydrogen.

14. The compound of any one of claims 1 to 13, or a pharmaceutically acceptable salt thereof, wherein R^{s} is selected from hydrogen, deuterium, methyl, ethyl, isopropyl, cyclopropyl, cyclohexyl, methoxy, ethoxy, isopropoxy, methylsulfonyl, ethylsulfonyl, hydroxy, methylcarbonyl, fluoro, chloro, difluoromethyl, trifluoromethyl, difluoromethoxy, trifluoromethoxy, amino, methylamino, dimethylamino, cyano, cyanomethyl, 2-cyanoprop-2-yl, cyanomethyloxy, hydroxymethyl, 1-hydroxyethyl, 1-hydroxy-1-methylethyl, methoxymethyl, methylaminomethyl, dimethylaminomethyl, methoxycarbonylaminomethyl, —CONH₂, methylaminocarbonyl, dimethylaminocarbonyl, 2-hydroxyethloxy, —O—(CH₂)₂NH₂, 2-methylaminoethyloxy, 2-dimethylaminoethyloxy, phenyl, phenoxy, 3-fluorophenoxy, 4-fluorophenoxy, 2-cyanophenoxy, 3-cyanophenyl, benzyl, 1-methyl-2-oxo-1,6-dihydropyridinyl, and morpholin-4-ylmethyl, R^{t} is hydrogen, fluoro, chloro, methoxy, difluoromethyl, difluoromethoxy, trifluoromethyl, trifluoromethoxy, or cyano, and R^{u} is hydrogen.

15. The compound of any one of claims 1 to 14, or a pharmaceutically acceptable salt thereof, wherein R⁴ and R⁵ together with the nitrogen atom to which they are attached form a ring selected from isoindolin-2-yl, 4-cyanoisoindolin-2-yl, 5-cyanoisoindolin-2-yl, 4-methoxyisoindolin-2-yl, 5-methoxyisoindolin-2-yl, 4-ethoxyisoindolin-2-yl, 4-isopropoxyisoindolin-2-yl, 5,6-dimethoxyisoindolin-2-yl, S-hydroxymethylisoindolin-2-yl, 4-difluoromethylisoindolin-2-yl, 5difluoromethylisoindolin-2-yl, 4-difluoromethoxyisoindolin-2-yl, 4-trifluoromethylisoindolin-2-yl, 5-trifluoromethylisoindolin-2-yl, 4-fluoroisoindolin-2-yl, 5-fluoroisoindolin-2-yl, 5,6-difluoroisoindolin-2-yl, 1-methylisoindolin-2-yl, 4-methylamino-carbonylisoindolin-2-yl, 4-dimethylaminocarbonyl-isoindolin-2-yl, 5-dimethylaminocarbonyl-isoindolin-2-yl, 4-(2~ (dimethylamino)ethoxy)-isoindolin-2-yl, 4-(2-(methylamino)ethoxy)-isoindolin-2-yl, 4-dimethylaminomethylisoindolin-2-yl, 5-dimethylaminomethylisoindolin-2-yl, 4-methylaminomethylisoindolin-2-yl, 4-dimethyl-aminoisoindolin-2-yl, 4-methylamino-isoindolin-2-yl, 3-phenylisoindolin-2-yl, 4-phenylisoindolin-2-yl, 4-phenoxyisoindolin-2-yl, 1-benzylisoindolin-2-yl, 4-benzylisoindolin-2-yl, 4-(3-fluoro-phenoxy)isoindolin-2-yl, 4-(4-fluorophenoxy)isoindolin-2-yl, 4-(2-cyanophenoxy)isoindolin-2-yl, 4-(3-cyanophenoxy)-isoindolin-2-yl, 4-cyclohexylisoindolin-2-yl, 5-(hydroxymethyl)isoindolin-2-yl, 4-(2-hydroxyethoxy)isoindolin-2-yl, 4-(2-aminoethoxy)isoindolin-2-yl, 5-(cyanomethyl)-isoindolin-2-yl, 4-(cyanomethyl)isoindolin-2-yl, indolin-1-yl, 2-methylindolin-1-yl, 4-cyanoindolin-1-yl, 5-cyanoindolin-1-yl, 6-cyanoindolin-1-yl, 4-hydroxymethylindolin-1-yl, 6-difluoromethylindolin-1-yl, 4-trifluoromethylindolin-1-yl, 5-trifluoromethylindolin-1-yl, 6-trifluoromethylindolin-1-yl, 6-methoxyindolin-1-yl, 5-chloroindolin-1-yl, 6-chloroindolin-1-yl, 6-dimethylaminoindolin-1-yl, 6-fluoroindolin-1-yl, 5,6-difluoroindolin-1-yl, 4,6-difluoroindolin-1-yl, 4,5-difluoroindolin-1-yl, 6-difluoromethoxyindolin-1-yl, 6-trifluoromethoxylindolin-1-yl, 4-((dimethylamino)methyl)-indolin-1-yl, 5-((dimethylamino)methyl)indolin-1-yl, 6-((dimethyl-amino)methyl)indolin-1-yl, 4-((methylamino)methyl)indolin-1-yl, 5-((methylamino)methyl)-indolin-1-yl, 6-((methylamino)-methyl)indolin-1-yl, 6-methylaminocarbonylindolin-1-yl, 6-aminocarbonyl-indolin-1-yl, 6-dimethylaminocarbonylindolin-1-yl, 4-(2-(methylamino)ethoxy)-indolin-1-yl, 6-(2-(dimethylamino)ethoxy) indolin-1-yl, 4-(hydroxymethyl)indolin-1-yl, S-phenylindolin-1-yl, 6-(1-methyl-6-oxo-1,6-dihydropyridin-2-yl)indolin-1-yl, 4-(1-hydroxyethyl)-indolin-1-yl, 4-(2-hydroxypropan-2-yl)indolin-1-yl, 6-(2-hydroxypropan-2-yl)indolin-1-yl, 4-(cyanomethyl)-indolin-1-yl, 6-(2-cyanopropan-2-yl)indolin-1-yl, 4-(morpholinomethyl)indolin-1-yl, 4-methoxycarbonyl-aminomethylindolin-1-yl, 7-(methylamino)indolin-1-yl, 6-(methylsulfonyl)indolin-1-yl, 6-chloroindolin-1-yl, 3,4-dihydroisoquinolin-2-yl, 1,2,4,5-tetrahydro-3H-benzo[d]azepin-3-yl, 2,3,4,5-tetrahydro-1H-benzo[b]azepin-1-yl, 2,3-dihydro-4H-benzo[b][1,4]oxazin-4-yl, 4-methyl-3,4-dihydroquinoxalin-1(2H)-yl, 1,2,3,5-tetrahydro-4H-benzo[e][1,4]diazepin-4-yl, 3,4-dihydroquinoxalin-1(2H)-yl, 2,3-dihydrobenzo[f][1,4]oxazepin-4 (5H)-yl, 1,3,4,5-tetrahydro-2H-benzo[c]azepine-2-yl, and 1-methyl-1,2,3,5-tetrahydro-4H-benzo[e][1,4]diazepin-4-yl, 2,3-dihydrobenzo[e][1,4]oxazepin-1(5H)-yl.

16. The compound of any one of claims 1 to 15, or a pharmaceutically acceptable salt thereof, wherein W is N.

17. The compound of any one of claims 1 to 16, or a pharmaceutically acceptable salt thereof, wherein R⁹ and R¹⁰ are independently alkyl, cycloalkyl, bridged cycloalkyl, spiro cycloalkyl, heterocyclyl, bridged heterocyclyl, or spiro heterocyclyl, wherein:
(A) cycloalkyl, bridged cycloalkyl, and spiro cycloalkyl of R⁹ and R¹⁰ are substituted with one or two R^{a}; and
(B) heterocyclyl, bridged heterocyclyl, and spiro heterocyclyl of R⁹ and R¹⁰ are substituted with R^{b}, R^{c}, and R^{d}.

18. The compound of any one of claims 1 to 17, or a pharmaceutically acceptable salt thereof, wherein R⁹ and R¹⁰ are alkyl.

19. The compound of any one of claims 1 to 17, or a pharmaceutically acceptable salt thereof, wherein R⁹ and R¹⁰ are independently cycloalkyl, bridged cycloalkyl, or spiro cycloalkyl wherein the cycloalkyl, bridged cycloalkyl, and spiro cycloalkyl of R⁹ and R¹⁰ are substituted with one or two R^{a}.

20. The compound of any one of claims 1 to 17 and 19, or a pharmaceutically acceptable salt thereof, wherein the one or two R^{a} are independently selected from hydrogen, deuterium, methyl, trideuteromethyl, fluoro, chloro, difluoromethyl, trifluoromethyl, and cyano.

21. The compound of any one of claims 1 to 17, or a pharmaceutically acceptable salt thereof, wherein R⁹ and R¹⁰ are independently heterocyclyl, bridged heterocyclyl, or spiro heterocyclyl, wherein the heterocyclyl, bridged heterocyclyl, and spiro heterocyclyl of R⁹ and R¹⁰ are substituted with R^{b}, R^{c}, and R^{d}.

22. The compound of any one of claims 1 to 17 and 19 to 21, or a pharmaceutically acceptable salt thereof, wherein the cycloalkyl and bridged cycloalkyl of R⁹ and R¹⁰ are selected from: 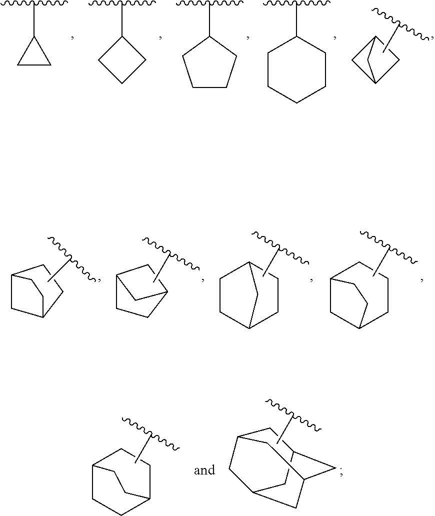
and the heterocyclyl and bridged heterocyclyl of R⁹ and R¹⁰ are selected from: 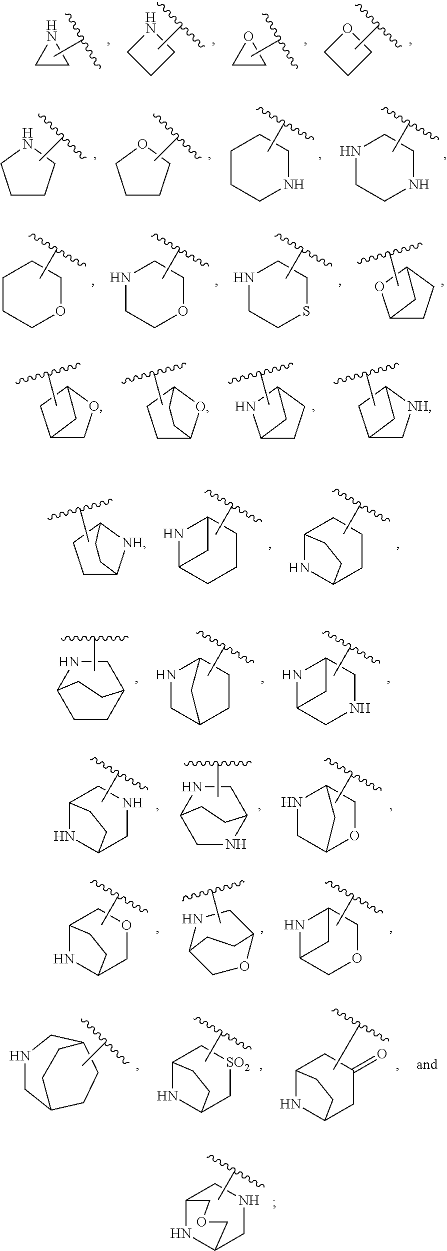
wherein each cycloalkyl and bridged cycloalkyl is substituted with R^{a}; and each heterocyclyl and bridged heterocyclyl is substituted with R^{b}, R^{c}, and R^{d}.

23. The compound of any one of claims 1 to 17, 19, 20, and 22, or a pharmaceutically acceptable salt thereof, wherein, wherein the bridged cycloalkyl of R⁹ and R¹⁰ are selected from: 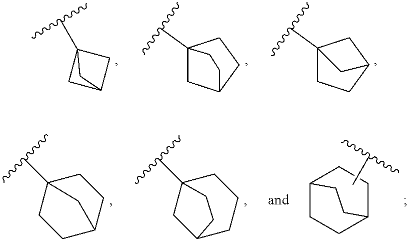
wherein each bridged cycloalkyl is substituted with R^{a}.

24. The compound of any one of claims 1 to 17, 19, 20, 22, and 23, or a pharmaceutically acceptable salt thereof, wherein R^{a} is hydrogen, deuterium, chloro, fluoro, difluoromethyl, or trifluoromethyl.

25. The compound of any one of claims 1 to 17, 19, 20, and 22 to 24, or a pharmaceutically acceptable salt thereof, wherein bridged cycloalkyl of R⁹ and R¹⁰ is bicyclo[1.1.1]pentan-1-yl, 3-fluorobicyclo[1.1.1]pentan-1-yl, 3-chlorobicyclo-[1.1.1]pentan-1-yl, 3-(hydroxymethyl)-bicyclo[1.1.1]pentan-1-yl, 3-(trifluoromethyl)-bicyclo[1.1.1]pentan-1-yl, 3-(difluoromethyl)-bicyclo[1.1.1]pentan-1-yl, and cycloalkyl of R⁹ and R¹⁰ is cyclobutyl, cyclopropyl, 1-(difluoromethyl)cyclobutyl, 1-(trifluoromethyl)cyclobutyl, 1-(difluoromethyl)-cyclopropyl, or 1-(trifluoromethyl)cyclopropyl.

26. The compound of any one of claims 1 to 17, 21, and 22, or a pharmaceutically acceptable salt thereof, wherein R^{b} and R^{c} are independently selected from hydrogen, methyl, fluoro, chloro, difluoromethyl, trifluoromethyl, 2,2-difluoroethyl, 2,2,2-trifluoroethyl, 3,3,3-trifluoropropyl, hydroxy, and cyano, and R^{d} is selected from hydrogen, methyl, trideuteromethyl, cyclopropyl, cyclobutyl, cyclopentyl, cyclohexyl, 2,2-difluoroethyl, 2,2,2-trifluoroethyl, 3,3,3-trifluoropropyl, amino, dimethylamino, diethylamino, 3,3-difluorocyclobutyl, 4,4-difluorocyclohexyl, 3-hydroxy-3-methylcyclobutyl, 3-cyano-3-methylcyclobutyl, oxetan-3-yl, tetrahydrofuran-2-yl, tetrahydropyran-4-yl, 1,1-dioxidothietan-3-yl, 1,1-dioxidotetrahydro-2H-thiopyran-4-yl, benzyl, phenyl, pyridin-2-yl, pyridin-3-yl, and pyridin-4-yl.

27. The compound of any one of claims 1 to 26, or a pharmaceutically acceptable salt thereof, wherein R⁷ is -Q-(alk¹)ₙ₁-SO₂R¹¹, -Q²-(alk³)ₙ₃-SO₂NR¹⁴R¹⁵, heterocyclyl, bicyclic heterocyclyl, spiro heterocyclyl, or spiro sulfoximinyl, wherein heterocyclyl, bicyclic heterocyclyl, spiro heterocyclyl, and spiro sulfoximinyl are substituted with R^{x}, R^{y}, and R^{y1}.

28. The compound of any one of claims 1 to 27, or a pharmaceutically acceptable salt thereof, wherein R⁷ is -Q-(alk¹)ₙ₁-SOR¹¹.

29. The compound of any one of claims 1 to 27, or a pharmaceutically acceptable salt thereof, wherein R⁷ is -Q²-(alk³)ₙ₃-SO₂NR¹⁴R¹⁵.

30. The compound of any one of claims 1 to 29, or a pharmaceutically acceptable salt thereof, wherein each of Q, Q¹, Q², Q³, Q⁴, Q⁵, and Q⁶ is cycloalkyl, bridged cycloalkyl, spiro cycloalkyl, heterocyclyl^{A}, bicyclic heterocyclyl^{A}, bridged heterocyclyl^{A}, spiro heterocyclyl^{A}, aryl, or heteroaryl, where each of the aforementioned rings is substituted with R^{v} and R^{w}.

31. The compound of any one of claims 1 to 30, or a pharmaceutically acceptable salt thereof, wherein each of n¹, n², n³, n⁴, n⁵, n⁶, and n⁷ is 0.

32. The compound of any one of claims 1 to 31, or a pharmaceutically acceptable salt thereof, wherein R⁷ is where:
(1) each of the Q, Q¹, Q², Q³, Q⁴, Q⁵, and Q⁶ is: 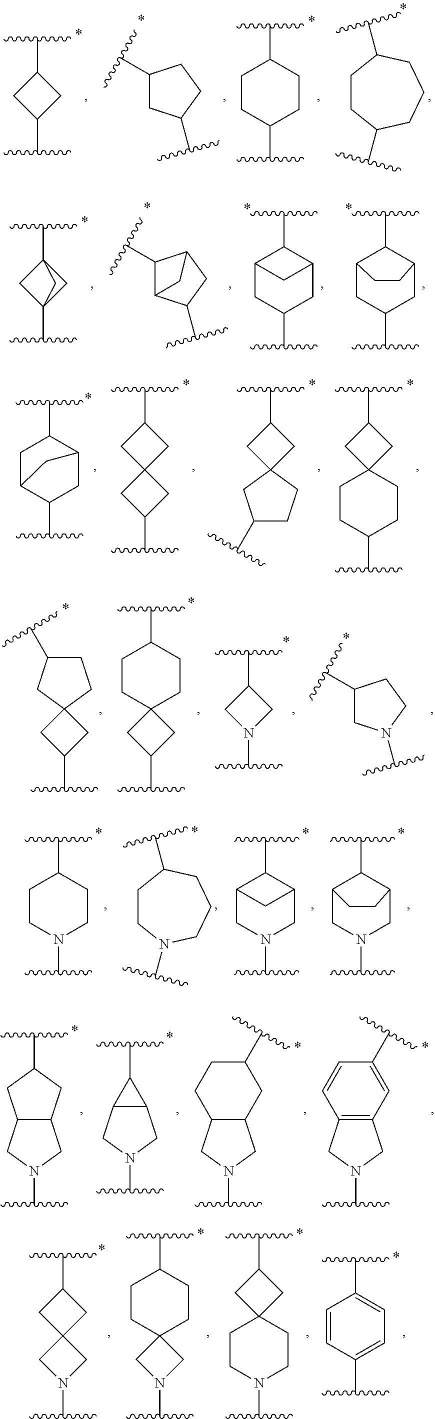 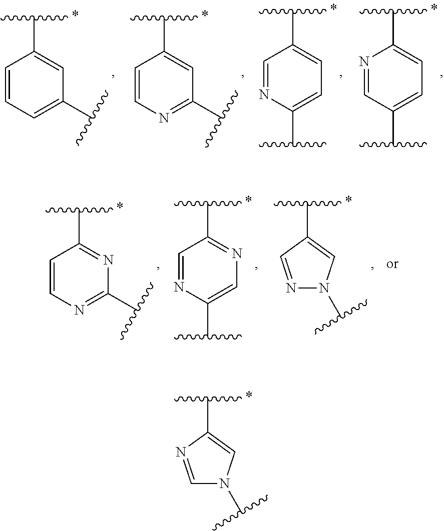
wherein each ring in (1) is substituted with R^{v} and R^{w}; and
(2) the heterocyclyl, bicyclic heterocyclyl, spiro heterocyclyl, bridged heterocyclyl, cyclylsulfoximinyl, spiro sulfoximinyl, aryl, heteroaryl, and fused heteroaryl of R⁷ are where:
heterocyclyl is: 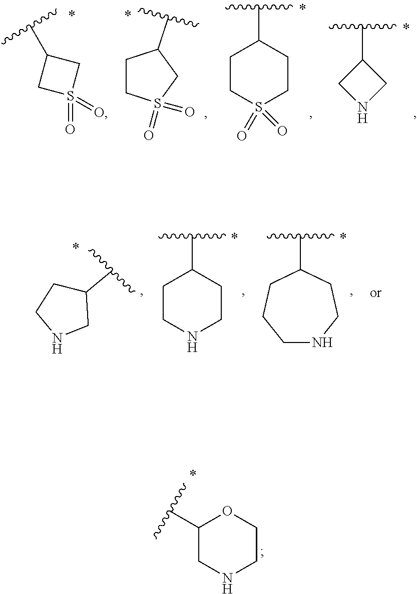
spiro heterocyclyl is: 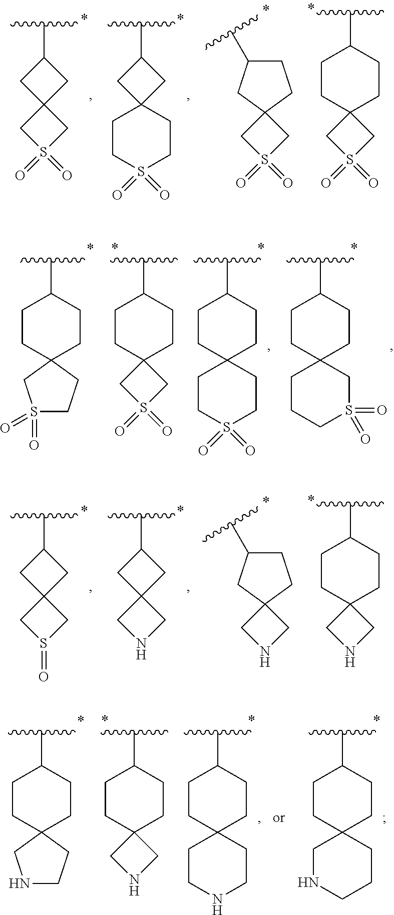
bridged heterocyclyl is: 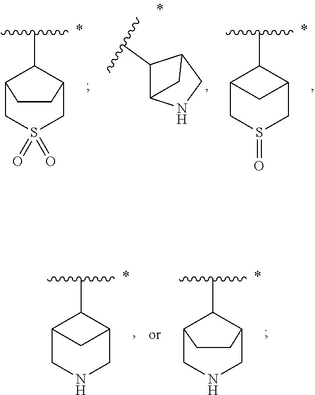
bicyclic heterocyclyl is: 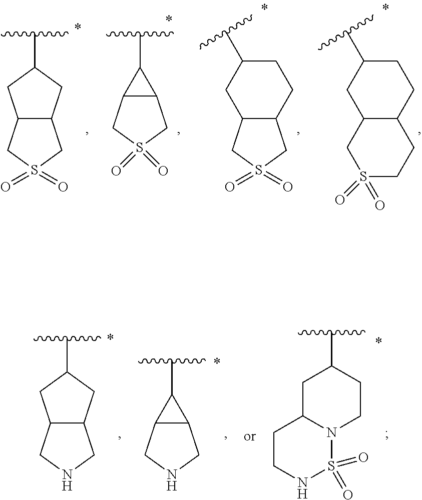
spiro sulfoximinyl is: 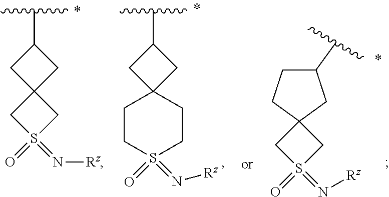
cyclylsulfoximinyl is: 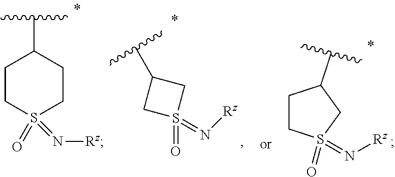
aryl is: 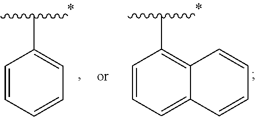 [SMILES: *c1ccccc1 ; *c1cccc2ccccc12]
heteroaryl is: 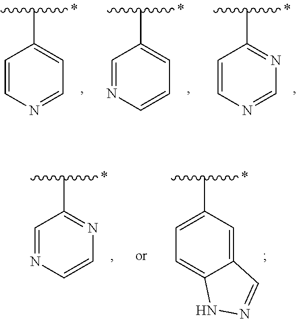 [SMILES: *c1ccncc1 ; *c1cccnc1 ; *c1ccncn1 ; *c1cnccn1 ; *c1ccc2[nH]ncc2c1]
fused heteroaryl is: 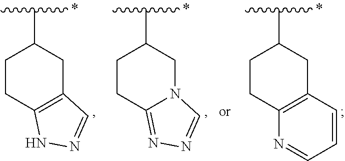
wherein each ring in (2) is substituted with R^{x}, R^{y}, and R^{y1}, and
wherein 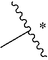
in (1) and (2) denotes the bond to NH of —NHR⁷ moiety of the compound of Formula (I); and 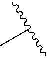
in (1) denotes bond to remainder of -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, -Q⁶-(alk⁶)ₙ₇-NR²¹SO₂R²².

33. The compound of any one of claims 1 to 32, or a pharmaceutically acceptable salt thereof, wherein:
(1) each of the Q, Q¹, Q², Q³, Q⁴, Q⁵, and Q⁶ is: 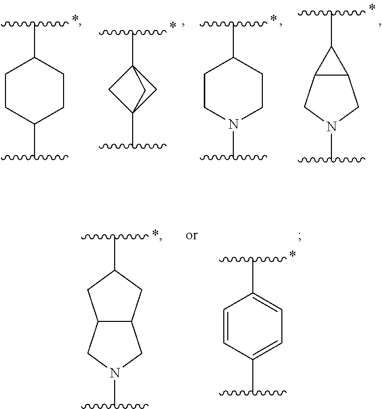
wherein each ring in (1) is substituted with R^{v} and R^{w} and 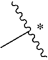
denotes the bond to NH of ~NHR⁷ moiety of the compound of Formula (I) and 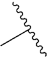
denotes bond to remainder of -Q-(alk¹)ₙ₁-SO₂R¹¹, -Q¹-(alk²)ₙ₂-SO(═NR¹²)R¹³, -Q²-(alk³)ₙ₃-SO₂NR¹⁴R¹⁵, -Q³-(alk⁴)ₙ₄-COR¹⁶, -Q⁴-(alk⁵)ₙ₅-CONR¹⁷R¹⁸, -Q⁵-(alk⁶)ₙ₆-NR¹⁹COR²⁰, -Q⁶-(alk⁶)ₙ₇-NR²¹SO₂R²²; and
(2) the heterocyclyl, bicyclic heterocyclyl, spiro heterocyclyl, spiro sulfoximinyl, aryl, and heteroaryl of R⁷ are where:
heterocyclyl is: 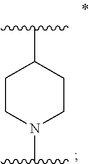 [SMILES: *C1CCN(*)CC1]
bicyclic heterocyclic is: 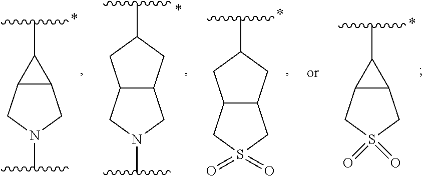 [SMILES: *C1C2CN(*)CC12 ; *C1CC2CN(*)CC2C1 ; *C1CC2CS(=O)(=O)CC2C1 ; *C1C2CS(=O)(=O)CC12]
spiro heterocyclyl is: 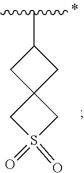 [SMILES: *C1CC2(C1)CS(=O)(=O)C2]
spirosulfoximinyl is: 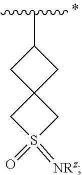
aryl is: 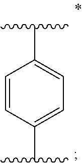 [SMILES: *c1ccc(*)cc1]
and heteroaryl is: 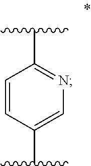 [SMILES: *c1ccc(*)nc1]
wherein each ring in (2) is substituted with R^{x}, R^{y}, and R^{y1} and 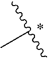
denotes the bond to NH of —NHR⁷ moiety of the compound of Formula (I) and 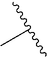
denotes attachment to one of R^{x}, R^{y}, and R^{y1}.

34. The compound of any one of claims 1 to 33, or a pharmaceutically acceptable salt thereof, wherein
R¹¹, R¹⁶, R²⁰, and R²² are independently selected from alkyl, fluoro, chloro, —CR²³═CR²⁴R²⁵ or heterocyclyl substituted with one to three substituents independently selected from hydrogen, alkyl, alkoxy, hydroxy, halo, haloalkyl, haloalkoxy, hydroxyalkyl, alkoxyalkyl, aminoalkyl, cyano, and heterocyclyl;
R¹³ is hydrogen, alkyl, haloalkyl, hydroxyalkyl, alkoxyalkyl, aminoalkyl, cycloalkyl; and
R¹⁴, R¹⁵, R¹⁷, and R¹⁸ are independently selected from hydrogen, alkyl, hydroxyalkyl, alkoxyalkyl, aminoalkyl, or heterocyclyl which is substituted with one to three substituents independently selected from hydrogen, alkyl, alkoxy, hydroxy, halo, haloalkyl, haloalkoxy, and cyano.

35. The compound of any one of claims 1 to 34, or a pharmaceutically acceptable salt thereof, wherein R⁷ is a ring of formula:

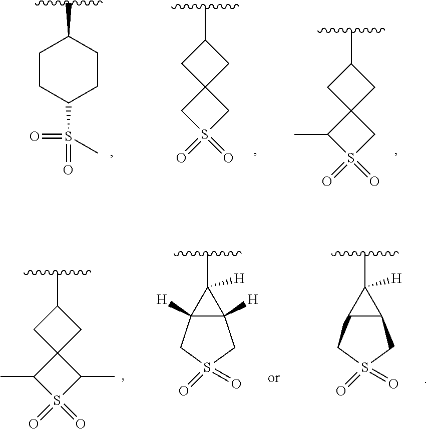

36. The compound of any one of claims 1 to 35, or a pharmaceutically acceptable salt thereof, wherein R⁶ is hydrogen.

37. A pharmaceutical composition comprising a compound of any one of claims 1-36, or a pharmaceutically acceptable salt thereof, and a pharmaceutically acceptable excipient.

38. A method of treating cancer in a patient which method comprises administering to the patient in need thereof, a therapeutically effective amount of a compound of any one of claims 1-36, or a pharmaceutically acceptable salt thereof, in a pharmaceutical composition of claim 37.

39. The method of claim 37, wherein the compound of any one of claims 1 to 36 or a pharmaceutically acceptable salt thereof, or the pharmaceutical composition of claim 37, is administered in combination with at least one other anticancer agent.

40. The method of claim 38 or 39, wherein the cancer is lung cancer, skin cancer, bladder cancer, breast cancer, cervical cancer, colorectal cancer, cancer of the small intestine, colon cancer, rectal cancer, cancer of the anus, endometrial cancer, gastric cancer, head and neck cancer, liver cancer, ovarian cancer, prostate cancer, testicular cancer, uterine cancer, esophageal cancer, gall bladder cancer, pancreatic cancer, stomach cancer, thyroid cancer, or parathyroid cancer.
